(19) United States
(12) Patent Application Publication
(10) Pub. No.: US 2023/0037682 A1
(43) Pub. Date: Feb. 9, 2023

(54) BINDING MOLECULES AGAINST CD3 AND USES THEREOF

(71) Applicant: NOVARTIS AG, Basel (CH)

(72) Inventors: Brian Walter GRANDA, Salisbury, MA (US); Daniel LENHERR-FREY, Uster (CH); Xuerui LUO, Pudong New Area, Shanghai (CN); Amy RAYO, Cambridge, MA (US); Fei ZHANG, Pudong New Area, Shanghai (CN); Jiquan ZHANG, Pudong New Area, Shanghai (CN)

(21) Appl. No.: 17/311,315

(22) PCT Filed: Dec. 4, 2019
(86) PCT No.: PCT/CN2019/122876

§ 371 Date: Jun. 4, 2021

(30) Foreign Application Priority Data
Dec. 4, 2018 (CN) PCT/CN2018/119074

(51) Int. Cl. C07K 16/28

(52) U.S. Cl. CPC C07K 16/2809; C07K 2317/24; C07K 2317/92; C07K 2317/75; C07K 2317/31

(57) ABSTRACT

Provided are CD3 binding molecules that specifically bind to CD3, for example monospecific binding molecules that specifically bind to CD3 and multispecific binding molecules (MBMs) that specifically bind to CD3 and a tumor-associated antigen, conjugates comprising the CD3 binding molecules, and pharmaceutical compositions comprising the CD3 binding molecules and conjugates. Provided are methods of using the CD3 binding molecules, conjugates, and pharmaceutical compositions to activate T cells in a subject, for example a subject having a cancer or autoimmune disease. Provided are recombinant host cells engineered to express the CD3 binding molecules and methods of producing the CD3 binding molecules by culturing the host cells under conditions in which the CD3 binding molecules are expressed.

### 1. CROSS-REFERENCE TO RELATED APPLICATIONS

This application claims the priority benefit of PCT application no. PCT/CN2018/119074, filed Dec. 4, 2018, the contents of which are incorporated herein by reference in their entirety.

### 2. SEQUENCE LISTING

The instant application contains a Sequence Listing which has been submitted electronically in ASCII format and is hereby incorporated by reference in its entirety. The ASCII copy, created on Dec. 3, 2019 is named NOV-010WO_SL.txt and is 782,920 bytes in size.

### 3. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

### 4. BACKGROUND

The conception of bi-specific and multi-specific antibodies arose from the idea that diseases are normally multi-factorial, and addressing more than one disease factor, but with a single antibody could increase efficacy. Cluster of differentiation 3 (CD3) is a homodimeric or heterodimeric antigen expressed on T cells in association with the T cell receptor complex (TCR) and is required for T cell activation. Antibodies against CD3 have been shown to cluster CD3 on T cells, thereby causing T cell activation in a manner similar to the engagement of the TCR by peptide-loaded MHC molecules. Anti-CD3 antibodies have been proposed for therapies involving the activation of T cells. Anti-CD3 antibodies have been used for the treatment of proliferative disorders such as cancer and for the treatment of autoimmune diseases. In addition, bispecific and multi-specific antibodies that are capable of binding CD3 and a target antigen have been proposed for therapeutic uses involving targeting T cell immune responses to tissues and cells expressing the target antigen. There are currently approved bispecific antibodies on the market, such as the CD19/CD3 BiTE, blinatumomab. However, bispecifics and multi-specific antibodies still face challenges of biodistribution, inhibitory microenvironments and antigen loss. As such, there is a need in the art for superior bispecific and multi-specific antibodies. Bispecific and multi-specific antigen-binding molecules that bind both CD3 and a target antigen would be useful in therapeutic settings in which specific targeting and T cell-mediated killing of cells that express the target antigen is desired.

There is a need for new CD3 binding molecules, e.g. antibodies and multispecific binding molecules, which bind CD3.

### 5. SUMMARY

The disclosure provides CD3 binding molecules that specifically bind to human CD3, e.g., antibodies, antigen-binding fragments thereof, and multispecific molecules that specifically bind to human CD3.

In one aspect, the disclosure provides monospecific CD3 binding molecules (e.g., antibodies and antigen-binding fragments thereof) comprising a CD3 antigen-binding domain or antigen-binding module (“ABM”). Exemplary CD3 binding molecules, which can be monospecific, are described in Section 7.2 and specific embodiments 1 to 456, infra.

In another aspect, the disclosure provides multispecific binding molecules (“MBMs”) comprising the CD3 ABMs, for example bispecific and multi-specific antibodies. Accordingly, in one aspect, the present disclosure is directed to bispecific and multi-specific antibodies comprising at least two separate antigen-binding domains or ABMs. In some aspects, the present disclosure provides bispecific and multi-specific binding molecules that engage a tumor-associated antigen (“TAA”) and CD3 and/or CD2 or other component of a TCR complex on T-cells.

In certain embodiments, the MBMs are bispecific binding molecules (“BBMs”). The BBMs comprise a first ABM that specifically binds to human CD3 (“ABM1” or “CD3 ABM”) and a second ABM that specifically binds to a second antigen (“ABM2”), e.g., a human TAA (sometimes referred to herein as a “TAA ABM”). The terms ABM1, ABM2, CD3 ABM, and TAA ABM are used merely for convenience and are not intended to convey any particular configuration of a BBM. Such multispecific molecules can be used to direct CD3+ effector T cells to TAA+ sites, thereby allowing the CD3+ effector T cells to attack and lyse the TAA+ cells and tumors. Features of exemplary MBMs are described in Sections 7.5 to 7.7 and specific embodiments 457 to 536, infra.

In certain embodiments, the MBMs are trispecific binding molecules (“TBMs”). The TBMs comprise a first ABM that specifically binds to human CD3 (“ABM1” or “CD3 ABM”), a second ABM (“ABM2”) that specifically binds to a second antigen, e.g., a human TAA, and a third ABM (“ABM3”) that specifically binds to a third antigen, e.g., a second human TAA or human CD2. TBMs that bind to (1) human CD3, (2) a TAA, and (3) CD2 are referred to herein as “Type 1 TBMs” for convenience. TBMs that bind to (1) human CD3, (2) a first TAA (sometimes referred to as “TAA 1”), and (3) a second TAA (sometimes referred to as “TAA 2”) are referred to herein as “Type 2 TBMs” for convenience. Because both TAA 1 and TAA 2 are tumor associated antigens, the designations of the tumor associated antigens of the disclosure as TAA 1 and TAA 2 are arbitrary—thus, any disclosure pertaining to TAA 1 is applicable to TAA 2 and vice versa, unless the context dictates otherwise.

In some embodiments, each antigen-binding module of a MBM is capable of binding its respective target at the same time as each of the one or more additional antigen-binding modules is bound to its respective target.

In the MBMs, each ABM (other than ABM1, which is immunoglobulin-based) can be immunoglobulin- or non-immunoglobulin-based, and therefore the MBMs can include immunoglobulin-based ABMs, non-immunoglobulin-based ABMs, or a combination thereof. Immunoglobulin-based ABMs that can be used in the MBMs are described in Section 7.3.1 and specific embodiments 1 to 469, infra. Non-immunoglobulin-based ABMs that can be used in the MBMs are described in Section 7.3.2 and specific embodiments 747 to 777, infra. Further features of exemplary ABMs that bind to a component of a TCR complex are described in Section 7.8, infra. Further features of exemplary ABMs that bind to CD2 are described in Section 7.9 and specific embodiments 746 to 789, infra. Further features of exemplary ABMs that bind to TAAs are described in Section 7.10 and specific embodiments 592 to 745 and 790 to 946, infra.

The ABMs of a MBM (or portions thereof) can be connected to each other, for example, by short peptide linkers or by an Fc domain. Methods and components for connecting ABMs to form a MBM are described in Section 7.4 and specific embodiments 947 to 1155, infra.

MBMs have at least two ABMs (i.e., a MBM is at least bivalent), but can also have more than two ABMs. For example, a MBM can have three ABMs (i.e., is trivalent), four ABMs (i.e., is tetravalent), five ABMs (i.e., is pentavalent), or six ABMs (i.e., is hexavalent). In some embodiments, a MBM has at least one ABM that can bind a TAA, at least one ABM that can bind CD3, and at least one ABM that can bind another antigen. Exemplary bivalent, trivalent, tetravalent, pentavalent, and hexavalent MBM configurations are described in Sections 7.5 to 7.7 and specific embodiments 477 to 536 and 554 to 590, infra.

The disclosure further provides nucleic acids encoding the CD3 binding molecules (e.g., MBMs) (either in a single nucleic acid or a plurality of nucleic acids) and recombinant host cells and cell lines engineered to express the nucleic acids and CD3 binding molecules (e.g., MBMs). Exemplary nucleic acids, host cells, and cell lines are described in Section 7.11 and specific embodiments 1439 to 1441, infra.

The present disclosure further provides drug conjugates comprising the CD3 binding molecules (e.g., MBMs). Such conjugates are referred to herein as “antibody-drug conjugates” or “ADCs” for convenience, notwithstanding that some of the ABMs can be non-immunoglobulin domains. Examples of ADCs are described in Section 7.12 and specific embodiments 1225 to 1262, infra.

Pharmaceutical compositions comprising the CD3 binding molecules (e.g., MBMs) and ADCs are also provided. Examples of pharmaceutical compositions are described in Section 7.14 and specific embodiment 1263, infra.

Further provided herein are methods of using the CD3 binding molecules (e.g., MBMs), the ADCs, and the pharmaceutical compositions, for example for treating proliferative conditions (e.g., cancers), on which TAAs are expressed. Exemplary methods are described in Section 7.15 and specific embodiments 1264 to 1437, infra.

The disclosure further provides methods of using the CD3 binding molecules (e.g., MBMs), the ADCs, and the pharmaceutical compositions in combination with other agents and therapies. Exemplary agents, therapies, and methods of combination therapy are described in Section 7.16 and specific embodiment 1438, infra.

### 6. BRIEF DESCRIPTION OF THE FIGURES

FIGS. 1A-1AH show exemplary BBM configurations. FIG. 1A illustrates components of the exemplary BBM configurations illustrated in FIGS. 1B-1AH. Not all regions connecting the different domains of each chain are illustrated (e.g., the linker connecting the VH and VL domains of an scFv, the hinge connecting the CH2 and CH3 domains of an Fc domain, etc., are omitted). FIGS. 1B-1F illustrate bivalent BBMs; FIGS. 1G-1Z illustrate trivalent BBMs; FIGS. 1AA-1AH illustrate tetravalent BBMs. In the BBMs, a variant CD58 domain can substitute for a Fab and/or scFv in any of the configurations illustrated.

FIGS. 2A-2V show exemplary TBM configurations. FIG. 2A illustrates components of the exemplary TBM configurations illustrated in FIGS. 2B-2V. Not all regions connecting the different domains of each chain are illustrated (e.g., the linker connecting the VH and VL domains of an scFv, the hinge connecting the CH2 and CH3 domains of an Fc, etc., are omitted). FIG. 2B-2P illustrates trivalent TBMs; FIGS. 2Q-2S illustrate tetravalent TBMs; FIG. 2T illustrates a pentavalent TBM, and FIGS. 2U-2V illustrate hexavalent TBMs. In the TBMs, a variant CD58 domain can substitute for a Fab and/or scFv in any of the configurations illustrated.

FIGS. 3A-3E show exemplary MBM configurations. FIG. 3A depicts a normal IgG format, FIG. 3B shows a BITE configuration, FIG. 3C is a bispecific configuration, FIG. 3D is a trispecific configuration and FIG. 3E is a tetraspecific configuration.

FIGS. 4A-4D are surface plasmon resonance (SPR/Biacore) measurements, showing the Kd for CD3. FIG. 4A: NOV292; FIG. 4B: sp34; FIG. 4C: NOV123; FIG. 4D: sp1c.

FIG. 5 shows the binding of anti-CD3 antibodies to cells transfected with human CD3.

FIG. 6 shows the binding of anti-CD3 antibodies to cells transfected with human CD3.

FIG. 7 shows the binding of anti-CD3 antibodies to cells transfected with human CD3.

FIG. 8 shows the binding of anti-CD3 antibodies to cells transfected with cynomolgus monkey (cyno) CD3.

FIG. 9 shows the binding of anti-CD3 antibodies to cells transfected with cynomolgus monkey (cyno) CD3.

FIG. 10 shows the binding of anti-CD3 antibodies to cells transfected with cynomolgus monkey (cyno) CD3.

FIG. 11 demonstrates agonist activation of CD3 by bispecific antibodies in a JNL cell model.

FIG. 12 demonstrates agonist activation of CD3 by bispecific antibodies in a JNL cell model.

FIG. 13 demonstrates agonist activation of CD3 by bispecific antibodies in a JNL cell model.

FIG. 14 demonstrates agonist activation of CD3 by bispecific antibodies in a JNL cell model.

FIG. 15 demonstrates agonist activation of CD3 by bispecific antibodies in a JNL cell model.

FIG. 16 shows the ability of an anti-CD19/anti-CD3 bispecific antibody to lyse target cells in a Redirected T-Cell Cytotoxcity (RTCC) assay.

FIG. 17 shows the ability of an anti-CD19/anti-CD3 bispecific antibody to lyse target cells in a Redirected T-Cell Cytotoxcity (RTCC) assay.

FIG. 18 shows the ability of anti-CD19/anti-CD3 bispecific antibodies to lyse target cells in a RTCC assay. “OKT3” and “sp34” refer to positive control bispecific antibodies.

FIG. 19 shows affinity of of anti-CD19/anti-CD3 bispecific antibodies for CD3+ T cells. “OKT3” and “sp34” refer to positive control bispecific antibodies.

FIG. 20 shows the ability of anti-CD19/anti-CD3 bispecific antibodies to lyse target cells in a RTCC assay. “OKT3,” “sp34,” and “H2C” refer to positive control bispecific antibodies.

FIG. 21 shows the ability of anti-CD19/anti-CD3 bispecific antibodies to lyse target cells in a RTCC assay. “OKT3,” “sp34,” and “H2C” refer to positive control bispecific antibodies.

FIG. 22 shows the ability of anti-CD19/anti-CD3 bispecific antibodies to lyse target cells in a RTCC assay. “OKT3” and “sp34” refer to positive control bispecific antibodies.

FIG. 23 shows affinity of of anti-CD19/anti-CD3 bispecific antibodies for CD3+ T cells. “OKT3” and “sp34” refer to positive control bispecific antibodies.

### 7. DETAILED DESCRIPTION

### 7.1. Definitions

As used herein, the following terms are intended to have the following meanings:

Antigen-binding module: The term “antigen-binding module” or “ABM” as used herein refers to a portion of a MBM of the disclosure that has the ability to bind to an antigen non-covalently, reversibly and specifically. An ABM can be immunoglobulin- or non-immunoglobulin-based. As used herein, the terms “ABM1” and “CD3 ABM” (and the like) refers to an ABM that binds specifically to CD3, and the term “ABM2” and “TAA ABM” (and the like) refer to an ABM that binds specifically to a tumor-associated antigen. The terms ABM1 and ABM2 etc., are used merely for convenience and are not intended to convey any particular configuration of a MBM.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies of the disclosure). The antibodies can be of any isotype/class (e.g., IgG, IgE, IgM, IgD, IgA and IgY) or subclass (e.g., IgG1, IgG2, IgG3, IgG4, IgA1 and IgA2).

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen-binding site or amino-terminus of the antibody. The N-terminus is a variable region and at the C-terminus is a constant region; the CH3 and CL domains actually comprise the carboxy-terminus of the heavy and light chain, respectively.

Antibody fragment: The term “antibody fragment” of an antibody as used herein refers to one or more portions of an antibody. In some embodiments, these portions are part of the contact domain(s) of an antibody. In some other embodiments, these portion(s) are antigen-binding fragments that retain the ability of binding an antigen non-covalently, reversibly and specifically, sometimes referred to herein as the “antigen-binding fragment”, “antigen-binding fragment thereof,” “antigen-binding portion”, and the like. Examples of binding fragments include, but are not limited to, single-chain Fvs (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Thus, the term “antibody fragment” encompasses both proteolytic fragments of antibodies (e.g., Fab and F(ab)2 fragments) and engineered proteins comprising one or more portions of an antibody (e.g., an scFv).

Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (for example, VH-CH1-VH-CH1) which, together with complementary light chain polypeptides (for example, VL-VC-VL-VC), form a pair of antigen-binding regions (Zapata et al., 1995, Protein Eng. 8:1057-1062; and U.S. Pat. No. 5,641,870).

Antigen-binding domain: The term “antigen-binding domain” refers a portion of a molecule that has the ability to bind to an antigen non-covalently, reversibly and specifically. Exemplary antigen-binding domains include antigen-binding fragments and portions of both immunoglobulin and non-immunoglobulin based scaffolds that retain the ability of binding an antigen non-covalently, reversibly and specifically. As used herein, the term “antigen-binding domain” encompasses antibody fragments that retain the ability of binding an antigen non-covalently, reversibly and specifically.

Half Antibody: The term “half antibody” refers to a molecule that comprises at least one ABM or ABM chain and can associate with another molecule comprising an ABM or ABM chain through, e.g., a disulfide bridge or molecular interactions (e.g., knob-in-hole interactions between Fc heterodimers). A half antibody can be composed of one polypeptide chain or more than one polypeptide chains (e.g., the two polypeptide chains of a Fab). In a preferred embodiment, a half-antibody comprises an Fc region.

An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, wherein said VL and VH domains form an ABM. Yet another example of a half antibody is a polypeptide comprising an scFv domain, a CH2 domain and a CH3 domain.

A half antibody might include more than one ABM, for example a half-antibody comprising (in N- to C-terminal order) an scFv domain, a CH2 domain, a CH3 domain, and another scFv domain.

Half antibodies might also include an ABM chain that when associated with another ABM chain in another half antibody forms a complete ABM.

Thus, a MBM can comprise one, more typically two, or even more than two half antibodies, and a half antibody can comprise one or more ABMs or ABM chains.

In some MBMs, a first half antibody will associate, e.g., heterodimerize, with a second half antibody. In other MBMs, a first half antibody will be covalently linked to a second half antibody, for example through disulfide bridges or chemical crosslinking. In yet other MBMs, a first half antibody will associate with a second half antibody through both covalent attachments and non-covalent interactions, for example disulfide bridges and knob-in-hole interactions.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, Md. (“Kabat” numbering scheme), Al-Lazikani et al., 1997, JMB 273:927-948 (“Chothia” numbering scheme) and ImMunoGenTics (IMGT) numbering (Lefranc, 1999, The Immunologist 7:132-136 (1999); Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Single Chain Fv or scFv: The term “single-chain Fv” or “scFv” as used herein refers to antibody fragments comprise the VH and VL domains of an antibody, wherein these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., (1994) Springer-Verlag, New York, pp. 269-315.

Diabody: The term “diabody” as used herein refers to small antibody fragments with two antigen-binding sites, typically formed by pairing of scFv chains. Each scFv comprises a heavy chain variable domain (VH) connected to a light chain variable domain (VL) in the same polypeptide chain (VH-VL, where the VH is either N-terminal or C-terminal to the VL). Unlike a typical scFv in which the VH and VL are separated by a linker that allows the VH and VL on the same polypeptide chain to pair and form an antigen-binding domain, diabodies typically comprise a linker that is too short to allow pairing between the VH and VL domains on the same chain, forcing the VH and VL domains to pair with the complementary domains of another chain and create two antigen-binding sites. Diabodies are described more fully in, for example, EP 404,097; WO 93/11161; and Hollinger et al., 1993, Proc. Natl. Acad. Sci. USA 90:6444-6448.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. By way of example and not limitation, the VH and VL can come together in any configuration described herein to form a half antibody, or can each be present on a separate half antibody and come together to form an antigen binding domain when the separate half antibodies associate, for example to form a MBM of the disclosure. When present on a single polypeptide chain (e.g., a scFv), the VH and be N-terminal or C-terminal to the VL.

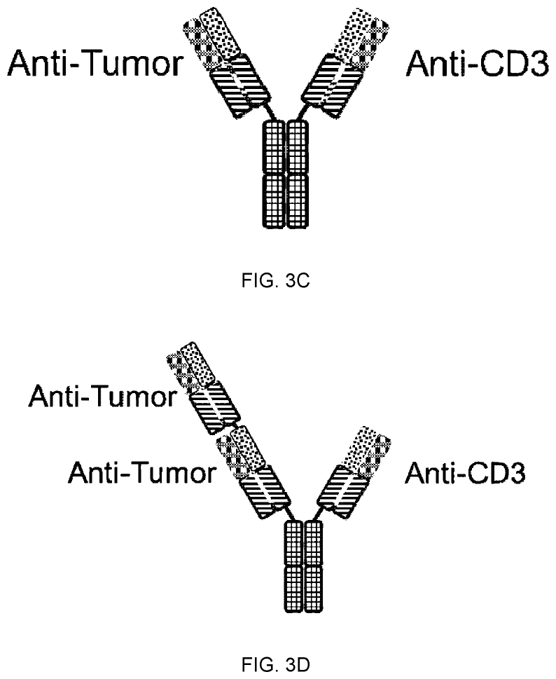

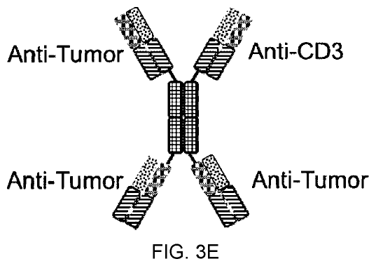

Multispecific binding molecules: The term “multispecific binding molecules” or “MBM” refers to molecules that comprise at least two antigen-binding domains, wherein at least one of the antigen binding domains is CD3 and least one antigen-binding domain which is specific for a TAA. The antigen-binding domains can each independently be an antibody fragment (e.g., scFv, Fab, nanobody), a ligand, or a non-antibody derived binder (e.g., fibronectin, Fynomer, DARPin). Representative MBMs are illustrated in FIGS. 3A-3E. MBMs can comprise one, two, three, four or even more polypeptide chains.

VH: The term “VH” refers to the variable region of an immunoglobulin heavy chain of an antibody, including but not limited to the heavy chain of an Fv, scFv, dsFv or Fab.

VL: The term “VL” refers to the variable region of an immunoglobulin light chain, including but not limited to the light chain of an Fv, scFv, dsFv or Fab.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked to produce a functional polypeptide. For example, in the context of a MBM of the disclosure, separate ABMs (or chains of an ABM) can be through peptide linker sequences. In the context of a nucleic acid encoding a fusion protein, such as a a polypeptide chain of a MBM of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Associated: The term “associated” in the context of a MBM refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional MBM in which ABM1, ABM2, etc. can bind their respective targets. Examples of associations that might be present in a MBM of the disclosure include (but are not limited to) associations between Fc regions in an Fc domain (homodimeric or, more preferably, heterodimeric as described in Section 7.4.1.5), associations between VH and VL regions in a Fab or Fv, and associations between CH1 and CL in a Fab.

ABM chain: Individual ABMs can exist as one (e.g., in the case of a scFv) polypeptide chain or form through the association of more than one polypeptide chains (e.g., in the case of a Fab). As used herein, the term “ABM chain” refers to all or a portion of an ABM that exists on a single polypeptide chain. The use of the term “ABM chain” is intended for convenience and descriptive purposes only and does not connote a particular configuration or method of production.

Host cell or recombinant host cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny can or can not be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing a MBM of the disclosure, a host cell is preferably a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Sequence identity: The term percent “identity,” in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or, in the case of a peptide or polypeptide, at least about 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, 1970, Adv. Appl. Math. 2:482c, by the homology alignment algorithm of Needleman and Wunsch, 1970, J. Mol. Biol. 48:443, by the search for similarity method of Pearson and Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., 2003, Current Protocols in Molecular Biology).

Two examples of algorithms that are suitable for determining percent sequence identity and sequence similarity are the BLAST and BLAST 2.0 algorithms, which are described in Altschul et al., 1977, Nuc. Acids Res. 25:3389-3402; and Altschul et al., 1990, J. Mol. Biol. 215:403-410, respectively. Software for performing BLAST analyses is publicly available through the National Center for Biotechnology Information.

The percent identity between two amino acid sequences can also be determined using the algorithm of Meyers and Miller, 1988, Comput. Appl. Biosci. 4:11-17, which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, 1970, J. Mol. Biol. 48:444-453) algorithm which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6.

Conservative Sequence Modifications: The term “conservative sequence modifications” refers to amino acid modifications that do not significantly affect or alter the binding characteristics of a MBM or a component thereof (e.g., an ABM or an Fc region). Such conservative modifications include amino acid substitutions, additions and deletions. Modifications can be introduced into a MBM of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a MBM of the disclosure can be replaced with other amino acid residues from the same side chain family and the altered MBM can be tested for, e.g., binding to target molecules and/or effective heterodimerization and/or effector function.

Mutation or modification: The terms “mutation” and “modification” in the context of a polypeptide as used herein can include substitution, addition or deletion of one or more amino acids.

Antibody Numbering Systems: In the present specification, the references to numbered amino acid residues in antibody domains are based on the EU numbering system unless otherwise specified. This system was originally devised by Edelman et al., 1969, Proc. Nat'l Acad. Sci. USA 63:78-85 and is described in detail in Kabat et al., 1991, in Sequences of Proteins of Immunological Interest, US Department of Health and Human Services, NIH, USA.

dsFv: The term “dsFv” refers to disulfide-stabilized Fv fragments. In a dsFv, a VH and VL are connected by an interdomain disulfide bond. To generate such molecules, one amino acid each in the framework region of in VH and VL are mutated to a cysteine, which in turn form a stable interchain disulfide bond. Typically, position 44 in the VH and position 100 in the VL are mutated to cysteines. See Brinkmann, 2010, Antibody Engineering 181-189, D01:10.1007/978-3-642-01147-4_14. The term dsFv encompasses both what is known in the art a dsFv (a molecule in which the VH and VL are connected by an interchain disulfide bond but not a linker peptide) or scdsFv (a molecule in which the VH and VL are connected by a linker as well as an interchain disulfide bond).

Tandem of VH Domains: The term “a tandem of VH domains (or VHs)” as used herein refers to a string of VH domains, consisting of multiple numbers of identical VH domains of an antibody. Each of the VH domains, except the last one at the end of the tandem, has its C-terminus connected to the N-terminus of another VH domain with or without a linker. A tandem has at least 2 VH domains, and in particular embodiments of the MBMs of the disclosure has 3, 4, 5, 6, 7, 8, 9, or 10 VH domains. The tandem of VH can be produced by joining the encoding nucleic acids of each VH domain in a desired order using recombinant methods with or without a linker (e.g., as described in Section 7.4.3) that enables them to be made as a single polypeptide chain. The N-terminus of the first VH domain in the tandem is defined as the N-terminus of the tandem, while the C-terminus of the last VH domain in the tandem is defined as the C-terminus of the tandem.

Tandem of VL Domains: The term “a tandem of VL domains (or VLs)” as used herein refers to a string of VL domains, consisting of multiple numbers of identical VL domains of an antibody. Each of the VL domains, except the last one at the end of the tandem, has its C-terminus connected to the N-terminus of another VL with or without a linker. A tandem has at least 2 VL domains, and in particular embodiments of the MBMs of the disclosure has 3, 4, 5, 6, 7, 8, 9, or 10 VL domains. The tandem of VL can be produced by joining the encoding nucleic acids of each VL domain in a desired order using recombinant methods with or without a linker (e.g., as described in Section 7.4.3) that enables them to be made as a single polypeptide chain. The N-terminus of the first VL domain in the tandem is defined as the N-terminus of the tandem, while the C-terminus of the last VL domain in the tandem is defined as the C-terminus of the tandem.

Monovalent: The term “monovalent” as used herein in the context of an antigen-binding molecule refers to an antigen-binding molecule that has a single antigen-binding domain.

Bivalent: The term “bivalent” as used herein in the context of an antigen-binding molecule refers to a MBM that has two antigen-binding domains, wherein one antigen-binding domains is CD3. The antigen-binding domains can be the same or different. Accordingly, a bivalent antigen-binding molecule can be monospecific or bispecific. An example of a bivalent MBM of the disclosure is shown schematically in FIG. 3C.

Trivalent: The term “trivalent” as used herein in the context of an antigen-binding molecule refers to an antigen-binding molecule that has three antigen-binding domains. Trivalent MBMs specifically bind to CD3, TAA and another antigen. Trivalent MBMs of the disclosure have at least three antigen-binding domains that each bind to a different antigen. An example of a trivalent MBM of the disclosure is shown schematically in FIG. 3D.

Tetravalent: The term “tetravalent” as used herein in the context of a MBM refers to an antigen-binding molecule that has four antigen-binding domains. The MBMs of the disclosure are tetravalent and specifically bind to CD3, a TAA and at least one other antigen. The tetravalent MBMs of the disclosure generally have two antigen-binding domains that bind to the same antigen (preferably the TAA) and at least one antigen-binding domain that binds CD3. An example of a tetravalent MBM of the disclosure is shown schematically in FIG. 3E.

Pentavalent: The term “pentavalent” as used herein in the context of a MBM refers to an antigen-binding molecule that has five antigen-binding domains. The MBMs of the disclosure are pentavalent and specifically bind to CD3, a TAA and three other antigens. Accordingly, the pentavalent MBMs of the disclosure generally have either (a) two pairs of antigen-binding domains that each bind to the same antigen and a single antigen-binding domain that binds to the third antigen or (b) three antigen-binding domains that bind to the same antigen and two antigen-binding domains that each bind to a separate antigen.

Hexavalent: The term “hexavalent” as used herein in the context of a MBM refers to an antigen-binding molecule that has six antigen-binding domains. The MBMs of the disclosure specifically bind to CD3, a TAA and at least one other antigen. The hexavalent MBMs of the disclosure generally have three pairs of antigen-binding domains that each bind to the same antigen, although different configurations (e.g., three antigen-binding domains that bind to the TAA, and at least one antigen-binding domain that binds to CD3, or three antigen-binding domains that bind to the TAA, and at least two antigen-binding domains that bind to CD3) are within the scope of the disclosure.

Specifically (or selectively) binds: The term “specifically (or selectively) binds” to an antigen or an epitope refers to a binding reaction that is determinative of the presence of a cognate antigen or an epitope in a heterogeneous population of proteins and other biologics. The binding reaction can be but need not be mediated by an antibody or antibody fragment, but can also be mediated by, for example, any type of ABM described in Section 7.3, such as a ligand, a DARPin, etc. An ABM of the disclosure typically also has a dissociation rate constant (KD) (koff/kon) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, or less than 10⁻⁹M, and binds to the target antigen with an affinity that is at least two-fold greater than its affinity for binding to a non-specific antigen (e.g., HSA). The term “specifically binds” does not exclude cross-species reactivity. For example, an antigen-binding module (e.g., an antigen-binding fragment of an antibody) that “specifically binds” to an antigen from one species can also “specifically bind” to that antigen in one or more other species. Thus, such cross-species reactivity does not itself alter the classification of an antigen-binding module as a “specific” binder. In certain embodiments, an antigen-binding module of the disclosure (e.g., ABM1, ABM2, etc.,) that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus. In other embodiments, the antigen-binding module of the disclosure (e.g., ABM1, ABM2, etc.,) does not have cross-species reactivity.

Monoclonal Antibody: The term “monoclonal antibody” as used herein refers to polypeptides, including antibodies, antibody fragments, molecules (including MBMs), etc. that are derived from the same genetic source.

Humanized: The term “humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. For further details, see Jones et al., 1986, Nature 321:522-525; Riechmann et al., 1988, Nature 332:323-329; and Presta, 1992, Curr. Op. Struct. Biol. 2:593-596. See also the following review articles and references cited therein: Vaswani and Hamilton, 1998, Ann. Allergy, Asthma & Immunol. 1:105-115; Harris, 1995, Biochem. Soc. Transactions 23:1035-1038; Hurle and Gross, 1994, Curr. Op. Biotech. 5:428-433.

Human Antibody: The term “human antibody” as used herein includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., 2000, J Mol Biol 296, 57-86. The structures and locations of immunoglobulin variable domains, e.g., CDRs, can be defined using well known numbering schemes, e.g., the Kabat numbering scheme, the Chothia numbering scheme, or a combination of Kabat and Chothia (see, e.g., Lazikani et al., 1997, J. Mol. Bio. 273:927 948; Kabat et al., 1991, Sequences of Proteins of Immunological Interest, 5th edit., NIH Publication no. 91-3242 U.S. Department of Health and Human Services; Chothia et al., 1987, J. Mol. Biol. 196:901-917; Chothia et al., 1989, Nature 342:877-883).

Human antibodies can include amino acid residues not encoded by human sequences (e.g., mutations introduced by random or site-specific mutagenesis in vitro or by somatic mutation in vivo, or a conservative substitution to promote stability or manufacturing). However, the term “human antibody”, as used herein, is not intended to include antibodies in which CDR sequences derived from the germline of another mammalian species, such as a mouse, have been grafted onto human framework sequences.

Chimeric Antibody: The term “chimeric antibody” (or antigen-binding fragment thereof) is an antibody molecule (or antigen-binding fragment thereof) in which (a) the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and can also be involved in autoimmune hypersensitivity. Effector function also includes Fc receptor (FcR)-mediated effector function, which can be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody can be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but can alter the geometry of the interaction rendering the effector mechanism ineffective as in non-productive binding. It is further envisaged that an effector function can also be altered by modifying a site not directly involved in effector molecule binding, but otherwise involved in performance of the effector function.

Recognize: The term “recognize” as used herein refers to an ABM that finds and interacts (e.g., binds) with its epitope.

Epitope: An epitope, or antigenic determinant, is a portion of an antigen recognized by an antibody or other antigen-binding domain as described herein. An epitope can be linear or conformational.

Nucleic Acid: The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions can be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

Vector: The term “vector” is intended to refer to a polynucleotide molecule capable of transporting another polynucleotide to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

Binding Sequences: In reference to the Tables (including subparts thereof), the term “binding sequences” means an ABM having a full set of CDRs, a VH-VL pair, or an scFv set forth in that table.

VH-VL or VH-VL Pair: In reference to a VH-VL pair, whether on the same polypeptide chain or on different polypeptide chains, the terms “VH-VL” and “VH-VL pair” are used for convenience and are not intended to convey any particular orientation, unless the context dictates otherwise. Thus, a scFv comprising a “VH-VL” or “VH-VL pair” can have the VH and VL domains in any orientation, for example the VH N-terminal to the VL or the VL N-terminal to the VH.

Polypeptide and Protein: The terms “polypeptide” and “protein” are used interchangeably herein to refer to a polymer of amino acid residues. The phrases also apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, binary tract cancer, bone cancer, endometrial cancer, eye cancer, fallopian tube cancer, genital tract cancers, large intestinal cancer, cancer of the meninges, oesophageal cancer, peritoneial cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like, e.g., any TAA-positive cancers of any of the foregoing types.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”).

Treat, Treatment, Treating: As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more MBMs of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments, the terms “treat”, “treatment” and “treating” refer to the reduction or stabilization of tumor size or cancerous cell count.

### 7.2. CD3 Binding Molecules

In one aspect, the disclosure provides CD3 binding molecules, including monospecific and multispecific molecules that bind to human CD3. In some embodiments, the CD3 binding molecule is a monospecific binding molecule. For example, the monospecific binding molecule can be an antibody or an antigen-binding fragment thereof (e.g., an antibody fragment, an scFv, a dsFv, a Fv, a Fab, an scFab, a (Fab′)2, or a single domain antibody (SDAB). In other embodiments, the CD3 binding molecule is a multispecific (e.g., bispecific) CD3 binding molecule (e.g., a bispecific antibody).

In some embodiments, the CD3 binding molecules are chimeric or humanized monoclonal antibodies. Chimeric and/or humanized antibodies, can be engineered to minimize the immune response by a human patient to antibodies produced in non-human subjects or derived from the expression of non-human antibody genes. Chimeric antibodies comprise a non-human animal antibody variable region and a human antibody constant region. Such antibodies retain the epitope binding specificity of the original monoclonal antibody, but can be less immunogenic when administered to humans, and therefore more likely to be tolerated by the patient. For example, one or all (e.g., one, two, or three) of the variable regions of the light chain(s) and/or one or all (e.g., one, two, or three) of the variable regions the heavy chain(s) of a mouse antibody (e.g., a mouse monoclonal antibody) can each be joined to a human constant region, such as, without limitation an IgG1 human constant region. Chimeric monoclonal antibodies can be produced by known recombinant DNA techniques. For example, a gene encoding the constant region of a non-human antibody molecule can be substituted with a gene encoding a human constant region (see Robinson et al., PCT Patent Publication PCT/US86/02269; Akira, et al., European Patent Application 184,187; or Taniguchi, M., European Patent Application 171,496). In addition, other suitable techniques that can be used to generate chimeric antibodies are described, for example, in U.S. Pat. Nos. 4,816,567; 4,978,775; 4,975,369; and 4,816,397.

Chimeric or humanized antibodies and antigen binding fragments thereof of the present disclosure can be prepared based on the sequence of a murine monoclonal antibody. DNA encoding the heavy and light chain immunoglobulins can be obtained from a murine hybridoma of interest and engineered to contain non-murine (e.g., human) immunoglobulin sequences using standard molecular biology techniques. For example, to create a chimeric antibody, the murine variable regions can be linked to human constant regions using known methods (see e.g., U.S. Pat. No. 4,816,567 to Cabilly et al.). To create a humanized antibody, the murine CDR regions can be inserted into a human framework using known methods. See e.g., U.S. Pat. No. 5,225,539 to Winter, and U.S. Pat. Nos. 5,530,101; 5,585,089; 5,693,762 and 6,180,370 to Queen et al.

A humanized antibody can be produced using a variety of known techniques, including but not limited to, CDR-grafting (see, e.g., European Patent No. EP 239,400; International Publication No. WO 91/09967; and U.S. Pat. Nos. 5,225,539, 5,530,101, and 5,585,089), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, antigen binding. These framework substitutions, e.g., conservative substitutions are identified by known methods, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323).

As provided herein, humanized antibodies or antibody fragments can comprise one or more CDRs from nonhuman immunoglobulin molecules and framework regions where the amino acid residues comprising the framework are derived completely or mostly from human germline. Multiple techniques for humanization of antibodies or antibody fragments are well-known and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332).

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework can be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993). In some embodiments, the framework region, e.g., all four framework regions, of the heavy chain variable region are derived from a VH4_4-59 germline sequence. In one embodiment, the framework region can comprise, one, two, three, four or five modifications, e.g., substitutions, e.g., conservative substitutions, e.g., from the amino acid at the corresponding murine sequence. In one embodiment, the framework region, e.g., all four framework regions of the light chain variable region are derived from a VK3_1.25 germline sequence. In one embodiment, the framework region can comprise, one, two, three, four or five modifications, e.g., substitutions, e.g., conservative substitutions, e.g., from the amino acid at the corresponding murine sequence.

In certain embodiments, the CD3 binding molecules comprise a heavy chain variable region from a particular germline heavy chain immunoglobulin gene and/or a light chain variable region from a particular germline light chain immunoglobulin gene. For example, such antibodies can comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence can contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody can be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pl and ablation variants herein; that is, the number of variants is generally low, prior to the introduction of the variants of the disclosure). In certain cases, the humanized antibody can display no more than 5, or even no more than 4, 3, 2, or 1 amino acid difference from the amino acid sequence encoded by the germline immunoglobulin gene (again, prior to the introduction of any skew, pl and ablation variants herein; that is, the number of variants is generally low, prior to the introduction of the variants of the disclosure).

In one embodiment, the parent antibody has been affinity matured. Structure-based methods can be employed for humanization and affinity maturation, for example as described in U.S. Ser. No. 11/004,590. Selection based methods can be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759. Other humanization methods can involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084.

In some embodiments, the CD3 binding molecule comprises an ABM which is a Fab. Fab domains can be produced by proteolytic cleavage of immunoglobulin molecules, using enzymes such as papain, or through recombinant expression. Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

In some embodiments, the CD3 binding molecule comprises an ABM which is a scFab. In an embodiment, the antibody domains and the linker in the scFab fragment have one of the following orders in N-terminal to C-terminal direction: a) VH-CH1-linker-VL-CL, or b) VL-CL-linker-VH-CH1. In some cases, VL-CL-linker-VH-CH1 is used.

In another embodiment, the antibody domains and the linker in the scFab fragment have one of the following orders in N-terminal to C-terminal direction: a) VH-CL-linker-VL-CH1 or b) VL-CH1-linker-VH-CL.

Optionally in the scFab fragment, additionally to the natural disulfide bond between the CL-domain and the CH1 domain, also the antibody heavy chain variable domain (VH) and the antibody light chain variable domain (VL) are disulfide stabilized by introduction of a disulfide bond between the following positions: i) heavy chain variable domain position 44 to light chain variable domain position 100, ii) heavy chain variable domain position 105 to light chain variable domain position 43, or iii) heavy chain variable domain position 101 to light chain variable domain position 100 (numbering according to EU index of Kabat).

Such further disulfide stabilization of scFab fragments is achieved by the introduction of a disulfide bond between the variable domains VH and VL of the single chain Fab fragments. Techniques to introduce unnatural disulfide bridges for stabilization for a single chain Fv are described e.g. in WO 94/029350, Rajagopal et al., 1997, Prot. Engin. 10:1453-59; Kobayashi et al., 1998, Nuclear Medicine & Biology, 25:387-393; and Schmidt, et al., 1999, Oncogene 18:1711-1721. In one embodiment, the optional disulfide bond between the variable domains of the scFab fragments is between heavy chain variable domain position 44 and light chain variable domain position 100. In one embodiment, the optional disulfide bond between the variable domains of the scFab fragments is between heavy chain variable domain position 105 and light chain variable domain position 43 (numbering according to EU index of Kabat).

In some embodiments, the CD3 binding molecule comprises an ABM which is a scFv. Single chain Fv antibody fragments comprise the VH and VL domains of an antibody in a single polypeptide chain, are capable of being expressed as a single chain polypeptide, and retain the specificity of the intact antibody from which it is derived. Generally, the scFv polypeptide further comprises a polypeptide linker between the VH and VL domain that enables the scFv to form the desired structure for target binding. Examples of linkers suitable for connecting the VH and VL chains of an scFV are the ABM linkers identified in Section 7.4.3, for example any of the linkers designated L1 through L58.

Unless specified, as used herein an scFv can have the VL and VH variable regions in either order, e.g., with respect to the N-terminal and C-terminal ends of the polypeptide, the scFv can comprise VL-linker-VH or can comprise VH-linker-VL.

To create an scFv-encoding nucleic acid, the VH and VL-encoding DNA fragments are operably linked to another fragment encoding a linker, e.g., encoding any of the linkers described in Section 7.4.3 (such as the amino acid sequence (Gly4^{˜}Ser)3 (SEQ ID NO: 47)), such that the VH and VL sequences can be expressed as a contiguous single-chain protein, with the VL and VH regions joined by the flexible linker (see e.g., Bird et al., 1988, Science 242:423-426; Huston et al., 1988, Proc. Natl. Acad. Sci. USA 85:5879-5883; McCafferty et al., 1990, Nature 348:552-554).

CD3 binding molecules can also comprise an ABM which is a Fv, a dsFv, a (Fab′)2, a single domain antibody (SDAB), a VH or VL domain, or a camelid VHH domain (also called a nanobody).

CD3 binding molecules can comprise a single domain antibody composed of a single VH or VL domain which exhibits sufficient affinity to CD3. In an embodiment, the single domain antibody is a camelid VHH domain (see, e.g., Riechmann, 1999, Journal of Immunological Methods 231:25-38; WO 94/04678).

Tables 1A to 1J-2 (collectively “Table 1”) list the sequences of exemplary CD3 binding sequences that can be included in CD3 binding molecules.

TABLE 1A
Consensus Group No. C1 Heavy Chain and
Light Chain CDR Consensus Sequences

| | | | SEQ ID |
|---|---|---|---|
| CDR | Binder | Sequence | NO: |
| CDR-H1 | C1-1 | GFX₁FX₂KX₃GMX₄ | 628 |
| CDR-H1 | C1-2 | GFX₁FX₂KX₃G | 629 |
| CDR-H1 | C1-3 | KX₃GMX₄ | 630 |
| CDR-H1 | C1-4 | GFX₁FX₂KX₃ | 631 |
| CDR-H2 | C1-5 | X₅IYYDSSX₆MYYADTVKG | 632 |
| CDR-H2 | C1-6 | YYDSSX₆ | 633 |
| CDR-H2 | C1-7 | IYYDSSX₆M | 634 |
| CDR-H3 | C1-8 | X₅₅X₈X₉DLDFDX₁₀ | 635 |
| CDR-H3 | C1-9 | AX₇X₅₅X₈X₉DLDFDX₁₀ | 636 |
| CDR-H3 | C1-10 | AALNSEYD | 637 |
| CDR-H3 | C1-11 | LNSEYD | 638 |
| CDR-L1 | C1-12 | RX₁₁SQSX₁₂X₁₃X₁₄SX₁₅X₁₆TTYFN | 639 |
| CDR-L1 | C1-13 | QSX₁₂X₁₃X₁₄SX₁₅TTY | 640 |
| CDR-L1 | C1-14 | SQSX₁₂X₁₃X₁₄SX₁₅X₁₆TTY | 641 |
| CDR-L1 | C1-15 | RX₁₁SQSX₁₂X₁₃X₁₄SX₁₅X₁₆ | 642 |
| CDR-L1 | C1-16 | SQSX₁₂X₁₃X₁₄S | 643 |
| CDR-L1 | C1-17 | QSX₁₂X₁₃X₁₄S | 644 |
| CDR-L2 | C1-18 | X₁₇X₁₈SX₁₉X₂₀X₂₁X₂₂ | 645 |
| CDR-L2 | C1-19 | X₁₇X₁₈ | 646 |
| CDR-L3 | C1-20 | LQX₂₃X₂₄X₂₅X₂₆PX₂₇T | 647 |
| CDR-L3 | C1-21 | X₂₃X₂₄X₂₅X₂₈PX₂₇ | 648 |
| CDR-L3 | C1-22 | LQX₂₃X₂₄X₂₅ | 649 |
| CDR-L3 | C1-23 | LQX₂₃X₂₄X₂₅X₂₆PX₂₇ | 650 |

X₁ is T or A;
X₂ is S or R;
X₃ is N, Y, or Q;
X₄ is H or S;
X₅ is M or L;
X₆ is K or R;
X₇ is S or K;
X₅₅ is F, Y, or S;
X₈ is W, Y, S, or T;
X₉ is W, Y, S, or T;
X₁₀ is H or Y;
X₁₁ is S or G;
X₁₂ is I or L;
X₁₃ is V or G;
X₁₄ is R or N;
X₁₅ is D, E, or L;
X₁₆ is G, N, or E;
X₁₇ is R or S;
X₁₈ is V or T;
X₁₉ is N or T;
X₂₀ is R or L;
X₂₁ is F or E;
X₂₂ is S or Y;
X₂₃ is S or Y;
X₂₄ is S or A;
X₂₅ is H or T;
X₂₆ is F or Y;
X₂₇ is W or Y

TABLE 1B
Consensus Group No. C2 Heavy Chain and
Light Chain CDR Consensus Sequences

| | | | SEQ ID |
|---|---|---|---|
| CDR | Binder | Sequence | NO: |
| CDR-H1 | C2-1 | GFSLTTYNX₂₈H | 651 |
| CDR-H1 | C2-2 | GFSLTTYN | 652 |
| CDR-H1 | C2-3 | TYNX₂₈H | 653 |
| CDR-H1 | C2-4 | GFSLTTY | 654 |
| CDR-H2 | C2-5 | RMRYSGDTSX₂₉X₃₀X₃₁ALX₃₂S | 655 |
| CDR-H2 | C2-6 | RYSGD | 656 |
| CDR-H2 | C2-7 | MRYSGDT | 657 |
| CDR-H3 | C2-8 | DPMYIPX₃₅YX₃₆YGVMNA | 658 |
| CDR-H3 | C2-9 | X₃₃X₃₄DPMYIPX₃₅YX₃₆YGVMNA | 659 |
| CDR-L1 | C2-10 | KX₃₇SQNIX₃₈X₃₉YLN | 660 |
| CDR-L1 | C2-11 | SQNIX₃₈X₃₉Y | 661 |
| CDR-L1 | C2-12 | QNIX₃₈X₃₉Y | 662 |
| CDR-L2 | C2-13 | NTX₄₀X₄₁LX₄₂AGVP | 663 |
| CDR-L2 | C2-14 | NTX₄₀X₄₁LX₄₂A | 664 |
| CDR-L2 | C2-15 | NTX₄₀ | 665 |
| CDR-L3 | C2-16 | LQHRSX₄₃YT | 666 |
| CDR-L3 | C2-17 | HRSX₄₃Y | 667 |

X₂₈ is V or I;
X₂₉ is F or Y;
X₃₀ is N or S;
X₃₁ is A or S;
X₃₂ is T or K;
X₃₃ is T or A;
X₃₄ is S or R;
X₃₅ is N or G;
X₃₆ is S or A;
X₃₇ is A, T, or S;
X₃₈ is N or D;
X₃₉ is N or K;
X₄₀ is D or N;
X₄₁ is H or N;
X₄₂ is Q or E;
X₄₃ is R, S, or G

TABLE 1C
Consensus Group No. C3 Heavy Chain and Light
Chain CDR Consensus Sequences

| | | | SEQ ID |
|---|---|---|---|
| CDR | Binder | Sequence | NO: |
| CDR-H1 | C3-1 | GYTFTSYYIY | 668 |
| CDR-H1 | C3-2 | GYTFTSYY | 669 |
| CDR-H1 | C3-3 | SYYIY | 670 |
| CDR-H1 | C3-4 | GYTFTSY | 671 |
| CDR-H2 | C3-5 | YIYPX₄₄X₄₅X₄₆X₄₇IYYSEX₄₈FKG | 672 |
| CDR-H2 | C3-6 | YPX₄₄X₄₅X₄₆X₄₇ | 673 |
| CDR-H2 | C3-7 | IYPX₄₄X₄₅X₄₆X₄₇I | 674 |
| CDR-H3 | C3-8 | X₄₉RPX₅₀TMMAPLX₅₁X₅₂ | 675 |
| CDR-H3 | C3-9 | PX₅₀TMMAPLX₅₁X₅₂ | 676 |
| CDR-L1 | C3-10 | RSSQSLX₅₃YSX₅₄GNTYLH | 677 |
| CDR-L1 | C3-11 | SQSLX₅₃YSX₅₄GNTY | 678 |
| CDR-L1 | C3-12 | QSLX₅₃YSX₅₄GNTY | 679 |
| CDR-L2 | C3-13 | RVSNRFS | 680 |
| CDR-L2 | C3-14 | RVS | 681 |
| CDR-L3 | C3-15 | FQSTHLPYT | 682 |
| CDR-L3 | C3-16 | STHLPY | 683 |

X₄₄ is G or A; X₄₅ is H or N; X₄₆ is D or G; X₄₇ is A or G; X₄₈ is N or K; X₄₉ iS V or A; X₅₀ is N or V; X₅₁ is A or V; X₅₂ is Y or F; X₅₃ is I or V; X₅₄ is I or H

TABLE 1D-1
CD3 Binders - Heavy Chain CDR sequences according to Kabat numbering scheme

| | | SEQ ID | | SEQ ID | | SEQ ID |
|---|---|---|---|---|---|---|
| Binder | CDR-H1 | NO: | CDR-H2 | NO: | CDR-H3 | NO: |
| NOV292 | KNGMH | 136 | MIYYDSSKMYY | 137 | FVWVDLDFDH | 138 |
| | | | ADTVKG | | | |
| NOV123 | SYYIY | 168 | YIYPGHDAIYYS | 169 | PNTMMAPLA | 170 |
| | | | ENFKG | | Y | |
| Sp10b | SYYIY | 168 | YIYPGHDAIYYS | 166 | PNTMMAPLA | 167 |
| | | | ENFKG | | Y | |
| NOV453 | TYNVH | 200 | RMRYSGDTSF | 201 | DPMYIPNYSY | 202 |
| | | | NAALTS | | GVMNA | |
| NOV229 | TYNVH | 232 | RMRYSGDTSF | 233 | DPMYIPNYSY | 234 |
| | | | NAALTS | | GVMNA | |
| NOV110 | SYYIY | 264 | YIYPANGGIYYS | 265 | PVTMMAPLV | 266 |
| | | | EKFKG | | F | |
| NOV832 | SYYIY | 296 | YIYPANGGIYYS | 297 | PVTMMAPLV | 298 |
| | | | EKFKG | | F | |
| N0V589 | KNGMH | 328 | MIYYDSSRMYY | 329 | FVWVDLDFDY | 330 |
| | | | ADTVKG | | | |
| N0V580 | TYNIH | 360 | RMRYSGDTSY | 361 | DPMYIPGYSY | 362 |
| | | | SSALKS | | GVMNA | |
| NOV567 | KYGMS | 392 | LIYYDSSKMNY | 393 | LNSEYD | 394 |
| | | | ADTVKG | | | |
| NOV221 | TYNIH | 424 | RMRYSGDTSY | 425 | DPMYIPGYSY | 426 |
| | | | SSALKS | | GVMNA | |
| CD3_sp11a_bkm1 | KNGMH | 136 | MIYYDSSKMYY | 134 | FVWVDLDFDH | 135 |
| | | | ADTVKG | | | |
| CD3_SP11a_bkm2 | KNGMH | 136 | MIYYDSSKMYY | 134 | FVWVDLDFDH | 135 |
| | | | ADTVKG | | | |
| CD3_sp11a_hz0 | KNGMH | 136 | MIYYDSSKMYY | 134 | FVWVDLDFDH | 135 |
| | | | ADTVKG | | | |
| CD3_SP11A_HZ1 | KNGMH | 136 | MIYYDSSKMYY | 134 | FVWVDLDFDH | 135 |
| | | | ADTVKG | | | |
| CD3_sp11a_ | KQGMH | 483 | MIYYDSSKMYY | 134 | FVWVDLDFDH | 135 |
| sansPTM_hz1 | | | ADTVKG | | | |
| CD3_sp11a_ | KQGMH | 483 | MIYYDSSKMYY | 134 | FVWVDLDFDH | 135 |
| sansPTM_rat | | | ADTVKG | | | |
| CD3_sp11a_VHVL_YY | KNGMH | 136 | MIYYDSSKMYY | 134 | FYYDLDFDH | 478 |
| | | | ADTVKG | | | |
| CD3_SP11A_VHVL_SS | KNGMH | 136 | MIYYDSSKMYY | 134 | FSSDLDFDH | 472 |
| | | | ADTVKG | | | |
| CD3_SP11A_VHVL_ | KNGMH | 136 | MIYYDSSKMYY | 134 | FWSDLDFDH | 476 |
| WS | | | ADTVKG | | | |
| CD3_sp11a_VHVLS_TW | KNGMH | 136 | MIYYDSSKMYY | 134 | FSVVDLDFDH | 473 |
| | | | ADTVKG | | | |
| CD3 SP11A_VHVL_T | KNGMH | 136 | MIYYDSSKMYY | 134 | FTTDLDFDH | 474 |
| T | | | ADTVKG | | | |
| CD3 SP11A_VHVL_T | KNGMH | 136 | MIYYDSSKMYY | 134 | FTVVDLDFDH | 475 |
| W | | | ADTVKG | | | |
| CD3_SP11A_VHVL_ | KNGMH | 136 | MIYYDSSKMYY | 134 | FVVTDLDFDH | 477 |
| WT | | | ADTVKG | | | |
| CD3_SP11A | KNGMH | 136 | MIYYDSSKMYY | 134 | FVWVDLDFDH | 135 |
| VH3_VLK_3 | | | ADTVKG | | | |
| CD3_sp11a_VH1_VK | KNQMH | 482 | MIYYDSSKMYY | 134 | FVWVDLDFDH | 135 |
| 2 | | | ADTVKG | | | |
| CD3_SP11A_VH3_VL | KNGMH | 136 | MIYYDSSKMYY | 134 | FVWVDLDFDH | 135 |
| K1 | | | ADTVKG | | | |
| CD3_SP11A_VH5_VK | KQGMH | 483 | MIYYDSSKMYY | 134 | FVWVDLDFDH | 135 |
| 2 | | | ADTVKG | | | |
| CD3_sp9aFW1_VL_V | TYNVH | 200 | RMRYSGDTSF | 198 | DPMYIPNYAY | 471 |
| H_S56G | | | NAALTS | | GVMNA | |
| CD3_SP9AFW4_VL_ | TYNVH | 200 | RMRYSGDTSF | 198 | DPMYIPNYAY | 471 |
| VH_S56G | | | NAALTS | | GVMNA | |
| CD3_sp9aFW1_VLVH | TYNVH | 200 | RMRYSGDTSF | 198 | DPMYIPNYAY | 471 |
| | | | NAALTS | | GVMNA | |
| CD3_sp9aFW4_VLVH | TYNVH | 200 | RMRYSGDTSF | 198 | DPMYIPNYAY | 471 |
| | | | NAALTS | | GVMNA | |
| CD3_sp9arabtor_VHVL | TYNVH | 200 | RMRYSGDTSF | 198 | DPMYIPNYAY | 471 |
| | | | NAALTS | | GVMNA | |
| CD3_sp9arabtor_VLVH | TYNVH | 200 | RMRYSGDTSF | 198 | DPMYIPNYAY | 471 |
| | | | NAALTS | | GVMNA | |
| CD3_sp11a VHVLY | KNGMH | 136 | MIYYDSSKMYY | 134 | FYYDLDFDH | 478 |
| Y_SANSPTM | | | ADTVKG | | | |
| CD3_sp11a_VHVL_Y | KNGMH | 136 | MIYYDSSKMYY | 134 | YYYDLDFDH | 627 |
| Y_SANSPTM_Y | | | ADTVKG | | | |
| CD3_sp11a_VHVL_Y | KNGMH | 136 | MIYYDSSKMYY | 134 | SYYDLDFDH | 619 |
| Y_SANSPTM_S | | | ADTVKG | | | |
| CD3_sp11a_VHVL_Y | KNGMH | 136 | MIYYDSSKMYY | 134 | YYYDLDFDH | 627 |
| Y_Y | | | ADTVKG | | | |
| CD3_sp11a_VHVL_Y | KNGMH | 136 | MIYYDSSKMYY | 134 | SYYDLDFDH | 619 |
| Y_s | | | ADTVKG | | | |
| CD3_sp11a_VHVL_S | KNGMH | 136 | MIYYDSSKMYY | 134 | FSSDLDFDH | 472 |
| S_SANSPTM | | | ADTVKG | | | |
| CD3_sp11a VHVL_S | KNGMH | 136 | MIYYDSSKMYY | 134 | YSSDLDFDH | 620 |
| S_SANSPTM_Y | | | ADTVKG | | | |
| CD3_sp11a VHVL_S | KNGMH | 136 | MIYYDSSKMYY | 134 | SSSDLDFDH | 613 |
| S_SANSPTM_S | | | ADTVKG | | | |
| CD3_sp11a_VHVL_S | KNGMH | 136 | MIYYDSSKMYY | 134 | YSSDLDFDH | 620 |
| S_Y | | | ADTVKG | | | |
| CD3_sp11a_VHVL_S | KNGMH | 136 | MIYYDSSKMYY | 134 | SSSDLDFDH | 613 |
| S_S | | | ADTVKG | | | |
| CD3_sp11a_VHVL_ | KNGMH | 136 | MIYYDSSKMYY | 134 | FSSDLDFDH | 472 |
| SS_SANSPTM | | | ADTVKG | | | |
| CD3_sp11a VHVL_ | KNGMH | 136 | MIYYDSSKMYY | 134 | YWSDLDFDH | 624 |
| WS_SANSPTM | | | ADTVKG | | | |
| CD3_sp11a_VHVL | KNGMH | 136 | MIYYDSSKMYY | 134 | SWSDLDFDH | 617 |
| WS_SANSPTM_S | | | ADTVKG | | | |
| CD3_sp11a_VHVL_ | KNGMH | 136 | MIYYDSSKMYY | 134 | YWSDLDFDH | 624 |
| WS_Y | | | ADTVKG | | | |
| CD3_sp11a_VHVL_ | KNGMH | 136 | MIYYDSSKMYY | 134 | SWSDLDFDH | 617 |
| WS_S | | | ADTVKG | | | |
| CD3_sp11a_VHVL | KNGMH | 136 | MIYYDSSKMYY | 134 | FWSDLDFDH | 476 |
| WS_SANSPTM | | | ADTVKG | | | |
| CD3_sp11a_VHVl_ | KNGMH | 136 | MIYYDSSKMYY | 134 | YSWDLDFDH | 621 |
| SW _SANSPTM_Y | | | ADTVKG | | | |
| CD3_sp11a_VHVL_ | KNGMH | 136 | MIYYDSSKMYY | 134 | SSWDLDFDH | 614 |
| SW _SANSPTM_S | | | ADTVKG | | | |
| CD3 sp11a_VHVL_ | KNGMH | 136 | MIYYDSSKMYY | 134 | YSWDLDFDH | 621 |
| SW_ Y | | | ADTVKG | | | |
| CD3_sp11a_VHVL_ | KNGMH | 136 | MIYYDSSKMYY | 134 | SSWDLDFDH | 614 |
| SW _S | | | ADTVKG | | | |
| CD3_sp11a_VHVL_ | KNGMH | 136 | MIYYDSSKMYY | 134 | FSVVDLDFDH | 473 |
| SW _SANSPTM | | | ADTVKG | | | |
| CD3_sp11a_VHVL_ | KNGMH | 136 | MIYYDSSKMYY | 134 | YTVVDLDFDH | 623 |
| TW _SANSPTM_Y | | | ADTVKG | | | |
| CD3_sp11a_VHVL_ | KNGMH | 136 | MIYYDSSKMYY | 134 | STVVDLDFDH | 616 |
| TW _SANSPTM_S | | | ADTVKG | | | |
| CD3 sp11a_VHVL_ | KNGMH | 136 | MIYYDSSKMYY | 134 | YTVVDLDFDH | 623 |
| TW_Y | | | ADTVKG | | | |
| CD3 sp11a_VHVL_ | KNGMH | 136 | MIYYDSSKMYY | 134 | STVVDLDFDH | 616 |
| TW_S | | | ADTVKG | | | |
| CD3_sp11a_VHVL_ | KNGMH | 136 | MIYYDSSKMYY | 134 | FTVVDLDFDH | 475 |
| TW_SANSPTM | | | ADTVKG | | | |
| CD3_sp11a_VHVL_ | KNGMH | 136 | MIYYDSSKMYY | 134 | YTTDLDFDH | 622 |
| TT_SANSPTM_Y | | | ADTVKG | | | |
| CD3_sp11a_VHVL_T | KNGMH | 136 | MIYYDSSKMYY | 134 | STTDLDFDH | 615 |
| T_SANSPTM_S | | | ADTVKG | | | |
| CD3_sp11a_VHVL_T | KNGMH | 136 | MIYYDSSKMYY | 134 | YTTDLDFDH | 622 |
| T_Y | | | ADTVKG | | | |
| CD3_sp11a_VHVL_T | KNGMH | 136 | MIYYDSSKMYY | 134 | STTDLDFDH | 615 |
| T_S | | | ADTVKG | | | |
| CD3_sp11a_VHVL_T | KNGMH | 136 | MIYYDSSKMYY | 134 | FTTDLDFDH | 474 |
| T_SANSPTM | | | ADTVKG | | | |
| CD3_SP11AVH3_VLK_ | KNGMH | 136 | MIYYDSSKMYY | 134 | YVWVDLDFDH | 626 |
| 3_Y | | | ADTVKG | | | |
| CD3_SP11AVH3_VLK_ | KNGMH | 136 | MIYYDSSKMYY | 134 | SVWVDLDFDH | 618 |
| 3_S | | | ADTVKG | | | |
| CD3_SP11AVH3_VLK_ | KNGMH | 136 | MIYYDSSKMYY | 134 | YVWVDLDFDH | 626 |
| 3_Y_PTM | | | ADTVKG | | | |
| CD3_SP11AVH3_VLK_ | KNGMH | 136 | MIYYDSSKMYY | 134 | SVWVDLDFDH | 618 |
| 3_S_PTM | | | ADTVKG | | | |
| CD3_SP11AVH3_VLK_ | KNGMH | 136 | MIYYDSSKMYY | 134 | YSWDLDFDH | 621 |
| 3_Y_SW | | | ADTVKG | | | |
| CD3_SP11AVH3_VLK | KNGMH | 136 | MIYYDSSKMYY | 134 | SSWDLDFDH | 614 |
| 3_S_SW | | | ADTVKG | | | |
| CD3_SP11AVH3_VLK | KNGMH | 136 | MIYYDSSKMYY | 134 | YSWDLDFDH | 621 |
| 3_Y_PTM_SW | | | ADTVKG | | | |
| CD3_SP11AVH3_VLK_ | KNGMH | 136 | MIYYDSSKMYY | 134 | SSWDLDFDH | 614 |
| 3_ S_SVVPTM | | | ADTVKG | | | |
| CD3_SP11AVH3_VLK_ | KNGMH | 136 | MIYYDSSKMYY | 134 | FSVVDLDFDH | 473 |
| SVVPTM | | | ADTVKG | | | |
| CD3_SP11AVH3_VLK_ | KNGMH | 136 | MIYYDSSKMYY | 134 | FSVVDLDFDH | 473 |
| 3_SW | | | ADTVKG | | | |
| CD3_sp11a_VH1_VK | KNQMH | 482 | MIYYDSSKMYY | 134 | YVWVDLDFDH | 626 |
| 2_Y | | | ADTVKG | | | |
| CD3_sp11a_VH1_VK | KNQMH | 482 | MIYYDSSKMYY | 134 | SVWVDLDFDH | 618 |
| 2_S | | | ADTVKG | | | |
| CD3_sp11a_VH1_VK | KNQMH | 482 | MIYYDSSKMYY | 134 | YVWVDLDFDH | 626 |
| 2_Y_PTM | | | ADTVKG | | | |
| CD3_sp11a_VH1_VK | KNQMH | 482 | MIYYDSSKMYY | 134 | SVWVDLDFDH | 618 |
| 2_S_PTM | | | ADTVKG | | | |
| CD3_sp11a_VH1_VK | KNQMH | 482 | MIYYDSSKMYY | 134 | YSWDLDFDH | 621 |
| 2_Y_SW | | | ADTVKG | | | |
| CD3_sp11a_VH1_VK | KNQMH | 482 | MIYYDSSKMYY | 134 | SSWDLDFDH | 614 |
| 2_S_SW | | | ADTVKG | | | |
| CD3_sp11a_VH1_VK | KNQMH | 482 | MIYYDSSKMYY | 134 | YSWDLDFDH | 621 |
| 2_Y_PTM | | | ADTVKG | | | |
| CD3_sp11a_VH1_VK | KNQMH | 482 | MIYYDSSKMYY | 134 | SSWDLDFDH | 614 |
| 2_S_PTM_SW | | | ADTVKG | | | |
| CD3_sp11a_VH1_VK | KNQMH | 482 | MIYYDSSKMYY | 134 | FSVVDLDFDH | 473 |
| 2_SW | | | ADTVKG | | | |
| CD3_sp11a_VH1_VK | KNQMH | 482 | MIYYDSSKMYY | 134 | FSVVDLDFDH | 473 |
| 2_SW_PTM | | | ADTVKG | | | |
| CD3_SP11A_VH3_VL | KNGMH | 136 | MIYYDSSKMYY | 134 | YVWVDLDFDH | 626 |
| K1_Y | | | ADTVKG | | | |
| CD3_SP11A_VH3_VL | KNGMH | 136 | MIYYDSSKMYY | 134 | SVWVDLDFDH | 618 |
| K1_S | | | ADTVKG | | | |
| CD3_SP11A_VH3_VL | KNGMH | 136 | MIYYDSSKMYY | 134 | YVWVDLDFDH | 626 |
| K1_Y_PTM | | | ADTVKG | | | |
| CD3_SP11A_VH3_VL | KNGMH | 136 | MIYYDSSKMYY | 134 | SVWVDLDFDH | 618 |
| K1_S_PTM | | | ADTVKG | | | |
| CD3_SP11A_VH3_VL | KNGMH | 136 | MIYYDSSKMYY | 134 | YSWDLDFDH | 621 |
| K1_Y_SW | | | ADTVKG | | | |
| CD3_SP11A_VH3_VL | KNGMH | 136 | MIYYDSSKMYY | 134 | SSWDLDFDH | 614 |
| K1_S_SW | | | ADTVKG | | | |
| CD3_SP11A_VH3_VL | KNGMH | 136 | MIYYDSSKMYY | 134 | YVWVDLDFDH | 626 |
| K1_Y_PTM | | | ADTVKG | | | |
| CD3_SP11A_VH3_VL | KNGMH | 136 | MIYYDSSKMYY | 134 | SSWDLDFDH | 614 |
| K1_S_PTM_SW | | | ADTVKG | | | |
| CD3_SP11A_VH3_VL | KNGMH | 136 | MIYYDSSKMYY | 134 | FSVVDLDFDH | 473 |
| KlPTM_SW | | | ADTVKG | | | |
| CD3_SP11A_VH3_VL | KNGMH | 136 | MIYYDSSKMYY | 134 | FSVVDLDFDH | 473 |
| K1_SW | | | ADTVKG | | | |
| CD3_SP11A_VH5_VK | KQGMH | 483 | MIYYDSSKMYY | 134 | YVWVDLDFDH | 626 |
| 2_Y | | | ADTVKG | | | |
| CD3_SP11A_VH5_VK | KQGMH | 483 | MIYYDSSKMYY | 134 | SVWVDLDFDH | 618 |
| 2_S | | | ADTVKG | | | |
| CD3_SP11A_VH5_VK | KQGMH | 483 | MIYYDSSKMYY | 134 | YVWVDLDFDH | 626 |
| 2_Y_PTM | | | ADTVKG | | | |
| CD3_SP11A_VH5_VK | KQGMH | 483 | MIYYDSSKMYY | 134 | SVWVDLDFDH | 618 |
| 2_S_PTM | | | ADTVKG | | | |
| CD3_SP11A_VH5_VK | KQGMH | 483 | MIYYDSSKMYY | 134 | YSWDLDFDH | 621 |
| 2_Y_SW | | | ADTVKG | | | |
| CD3_SP11A_VH5_VK | KQGMH | 483 | MIYYDSSKMYY | 134 | SSWDLDFDH | 614 |
| 2_S_SW | | | ADTVKG | | | |
| CD3_SP11A_VH5_VK | KQGMH | 483 | MIYYDSSKMYY | 134 | YSWDLDFDH | 621 |
| 2_Y_PTM_SW | | | ADTVKG | | | |
| CD3_SP11A_VH5_VK | KQGMH | 483 | MIYYDSSKMYY | 134 | SSWDLDFDH | 614 |
| 2_S_PTM_SW | | | ADTVKG | | | |
| CD3_SP11A_VH5_VK | KQGMH | 483 | MIYYDSSKMYY | 134 | FSVVDLDFDH | 473 |
| 2_PTM_SW | | | ADTVKG | | | |
| CD3_SP11A_VH5_VK | KQGMH | 483 | MIYYDSSKMYY | 134 | FSVVDLDFDH | 473 |
| 2_SW | | | ADTVKG | | | |

TABLE 1D-2
CD3 Binders - Light Chain CDR sequences according to Kabat numbering scheme

| | | SEQ ID | | SEQ ID | | SEQ ID |
|---|---|---|---|---|---|---|
| Binder | CDR-L1 | NO: | CDR-L2 | NO: | CDR-L3 | NO: |
| NOV292 | RSSQSLVRSD | 152 | RVSNR | 153 | LQSSHFP | 154 |
| | GTTYFN | | FS | | WT | |
| NOV123 | RSSQSLIYSIGN | 184 | RVSNR | 185 | FQSTHLP | 186 |
| | TYLH | | FS | | YT | |
| Sp10b | RSSQSLIYSIGN | 181 | RVSNR | 150 | FQSTHLP | 183 |
| | TYLH | | FS | | YT | |
| NOV453 | KASQNINNYLN | 216 | NTDHL | 217 | LQHRSR | 218 |
| | | | QA | | YT | |
| NOV229 | KASQNINNYLN | 248 | NTDHL | 249 | LQHRSR | 250 |
| | | | QA | | YT | |
| NOV110 | RSSQSLVYSHG | 280 | RVSNR | 281 | FQSTHLP | 282 |
| | NTYLH | | FS | | YT | |
| NOV832 | RSSQSLVYSHG | 312 | RVSNR | 313 | FQSTHLP | 314 |
| | NTYLH | | FS | | YT | |
| NOV589 | RSSQSLVRSD | 344 | RVSNR | 345 | LQSSHFP | 346 |
| | GTTYFN | | FS | | WT | |
| NOV580 | KTSQNIDKYLN | 376 | NTNNL | 377 | LQHRSSY | 378 |
| | | | EA | | T | |
| NOV567 | RGSQSIGNSLN | 408 | STSTL | 409 | LQYATYP | 410 |
| | | | EY | | YT | |
| NOV221 | KSSQNIDKYLN | 440 | NTNNL | 441 | LQHRSG | 442 |
| | | | EA | | YT | |
| CD3_sp11a_bkm1 | RSSQSLVRSD | 149 | RVSNR | 150 | LQSSHFP | 151 |
| | GTTYFN | | FS | | WT | |
| CD3_SP11a_bkm2 | RSSQSLVRSD | 149 | RVSNR | 150 | LQSSHFP | 151 |
| | GTTYFN | | FS | | WT | |
| CD3_sp11a_hz0 | RSSQSLVRSD | 149 | RVSNR | 150 | LQSSHFP | 151 |
| | GTTYFN | | FS | | WT | |
| CD3_SP11A_HZ1 | RSSQSLVRSD | 149 | RVSNR | 150 | LQSSH | 484 |
| | GTTYFN | | FS | | | |
| CD3_sp11a_sansPTM_hz1 | RSSQSLVRSEG | 487 | RVSNR | 150 | LQSSHFP | 151 |
| | TTYFN | | FS | | WT | |
| CD3_sp11a_sansPTM_rat | RSSQSLVRSEG | 487 | RVSNR | 150 | LQSSHFP | 151 |
| | TTYFN | | FS | | WT | |
| CD3_sp11a_VHVL_YY | RSSQSLVRSD | 149 | RVSNR | 150 | LQSSHFP | 151 |
| | GTTYFN | | FS | | WT | |
| CD3_SP11A_VHVL_SS | RSSQSLVRSD | 149 | RVSNR | 150 | LQSSHFP | 151 |
| | GTTYFN | | FS | | WT | |
| CD3_SP11A_VHVL_WS | RSSQSLVRSD | 149 | RVSNR | 150 | LQSSHFP | 151 |
| | GTTYFN | | FS | | WT | |
| CD3_sp11a_VHVL_SW | RSSQSLVRSD | 149 | RVSNR | 150 | LQSSHFP | 151 |
| | GTTYFN | | FS | | WT | |
| CD3_SP11A_VHVL_TT | RSSQSLVRSD | 149 | RVSNR | 150 | LQSSHFP | 151 |
| | GTTYFN | | FS | | WT | |
| CD3_SP11A_VHVL_TW | RSSQSLVRSD | 149 | RVSNR | 150 | LQSSHFP | 151 |
| | GTTYFN | | FS | | WT | |
| CD3_SP11A_VHVL_VVT | RSSQSLVRSD | 149 | RVSNR | 150 | LQSSHFP | 151 |
| | GTTYFN | | FS | | WT | |
| CD3_SP11A VH3_VLK_3 | RSSQSLVRSEG | 487 | RVSNR | 150 | LQSSHFP | 151 |
| | TTYFN | | FS | | WT | |
| CD3_sp11a_VH1_VK2 | RSSQSLVRSD | 149 | RVSNR | 150 | LQSSHFP | 151 |
| | GTTYFN | | FS | | WT | |
| CD3_SP11A_VH3_VLK1 | RSSQSLVRSEG | 487 | RVSNR | 150 | LQSSHFP | 151 |
| | TTYFN | | FS | | WT | |
| CD3_SP11A_VH5_VK2 | RSSQSLVRSD | 149 | RVSNR | 150 | LQSSHFP | 151 |
| | GTTYFN | | FS | | WT | |
| CD3_sp9aFW1_VL_VH_S5 | KASQNINNYLN | 213 | NTDHL | 214 | LQHRSR | 215 |
| 6G | | | QA | | YT | |
| CD3_SP9AFW4_VL_VH_S | KASQNINNYLN | 213 | NTDHL | 214 | LQHRSR | 215 |
| 56G | | | QA | | YT | |
| CD3_sp9aFW1_VLVH | KASQNINNYLN | 213 | NTDHL | 214 | LQHRSR | 215 |
| | | | QA | | YT | |
| CD3_sp9aFW4_VLVH | KASQNINNYLN | 213 | NTDHL | 214 | LQHRSR | 215 |
| | | | QA | | YT | |
| CD3_sp9arabtor_VHVL | KASQNINNYLN | 213 | NTDHL | 214 | LQHRSR | 215 |
| | | | QA | | YT | |
| CD3_sp9arabtor_VLVH | KASQNINNYLN | 213 | NTDHL | 214 | LQHRSR | 215 |
| | | | QA | | YT | |
| CD3_sp11a_VHVL_YY_SA | RSSQSLVRSEG | 487 | RVSNR | 150 | LQSSHFP | 151 |
| NSPTM | TTYFN | | FS | | WT | |
| CD3_sp11a_VHVL_YY_SA | RSSQSLVRSEG | 487 | RVSNR | 150 | LQSSHFP | 151 |
| NSPTM_Y | TTYFN | | FS | | WT | |
| CD3_sp11a_VHVL_YY_SA | RSSQSLVRSEG | 487 | RVSNR | 150 | LQSSHFP | 151 |
| NSPTM_S | TTYFN | | FS | | WT | |
| CD3_sp11a_VHVL_YY_Y | RSSQSLVRSD | 149 | RVSNR | 150 | LQSSHFP | 151 |
| | GTTYFN | | FS | | WT | |
| CD3_sp11a_VHVL_YY_s | RSSQSLVRSD | 149 | RVSNR | 150 | LQSSHFP | 151 |
| | GTTYFN | | FS | | WT | |
| CD3_sp11a_VHVL_SS_SA | RSSQSLVRSEG | 487 | RVSNR | 150 | LQSSHFP | 151 |
| NSPTM | TTYFN | | FS | | WT | |
| CD3_sp11a_VHVL_SS_SA | RSSQSLVRSEG | 487 | RVSNR | 150 | LQSSHFP | 151 |
| NSPTM Y | TTYFN | | FS | | WT | |
| CD3_sp-11a_VHVL_SS_SA | RSSQSLVRSEG | 487 | RVSNR | 150 | LQSSHFP | 151 |
| NSPTM_S | TTYFN | | FS | | WT | |
| CD3_sp11a_VHVL_SS_Y | RSSQSLVRSD | 149 | RVSNR | 150 | LQSSHFP | 151 |
| | GTTYFN | | FS | | WT | |
| CD3_sp11a_VHVL_SS_S | RSSQSLVRSD | 149 | RVSNR | 150 | LQSSHFP | 151 |
| | GTTYFN | | FS | | WT | |
| CD3_sp11a VHVL_SS_ | RSSQSLVRSEG | 487 | RVSNR | 150 | LQSSHFP | 151 |
| SANSPTM | TTYFN | | FS | | WT | |
| CD3_sp11aVHVL_WS_ | RSSQSLVRSEG | 487 | RVSNR | 150 | LQSSHFP | 151 |
| SANSPTM_Y | TTYFN | | FS | | WT | |
| CD3_sp11a_VHVL_WS_ | RSSQSLVRSEG | 487 | RVSNR | 150 | LQSSHFP | 151 |
| SANSPTM_S | TTYFN | | FS | | WT | |
| CD3_sp11a1_VHVL_WS_Y | RSSQSLVRSD | 149 | RVSNR | 150 | LQSSHFP | 151 |
| | GTTYFN | | FS | | WT | |
| CD3_sp11a_VHVL_WS_S | RSSQSLVRSD | 149 | RVSNR | 150 | LQSSHFP | 151 |
| | GTTYFN | | FS | | WT | |
| CD3_sp11a_VHVL_WS_ | RSSQSLVRSEG | 487 | RVSNR | 150 | LQSSHFP | 151 |
| SANSPTM | TTYFN | | FS | | WT | |
| CD3_sp11a_VHVL_SW_ | RSSQSLVRSEG | 487 | RVSNR | 150 | LQSSHFP | 151 |
| SANSPTM_Y | TTYFN | | FS | | WT | |
| CD3_sp11a_VHVL_SW_ | RSSQSLVRSEG | 487 | RVSNR | 150 | LQSSHFP | 151 |
| SANSPTM_S | TTYFN | | FS | | WT | |
| CD3_sp11a_VHVL_SW_Y | RSSQSLVRSD | 149 | RVSNR | 150 | LQSSHFP | 151 |
| | GTTYFN | | FS | | WT | |
| CD3_sp11a_VHVL_SW_S | RSSQSLVRSD | 149 | RVSNR | 150 | LQSSHFP | 151 |
| | GTTYFN | | FS | | WT | |
| CD3_sp11a_VHVL_ SW_ | RSSQSLVRSEG | 487 | RVSNR | 150 | LQSSHFP | 151 |
| SANSPTM | TTYFN | | FS | | WT | |
| CD3_sp11a_VHVL_TW_ | RSSQSLVRSEG | 487 | RVSNR | 150 | LQSSHFP | 151 |
| SANSPTM_Y | TTYFN | | FS | | WT | |
| CD3_sp11a_VHVL_TW_ | RSSQSLVRSEG | 487 | RVSNR | 150 | LQSSHFP | 151 |
| SANSPTM_S | TTYFN | | FS | | WT | |
| CD3_sp11a_VHVL_TW_Y | RSSQSLVRSD | 149 | RVSNR | 150 | LQSSHFP | 151 |
| | GTTYFN | | FS | | WT | |
| CD3_sp11a_VHVL_TW_S | RSSQSLVRSD | 149 | RVSNR | 150 | LQSSHFP | 151 |
| | GTTYFN | | FS | | WT | |
| CD3_sp11a_VHVL_TW_ | RSSQSLVRSEG | 487 | RVSNR | 150 | LQSSHFP | 151 |
| SANSPTM | TTYFN | FS | WT | | | |
| CD3_sp11a_VHVL_TT_ | RSSQSLVRSEG | 487 | RVSNR | 150 | LQSSHFP | 151 |
| SANSPTM_Y | TTYFN | | FS | | WT | |
| CD3_sp11a_VHVL_TT_SA | RSSQSLVRSEG | 487 | RVSNR | 150 | LQSSHFP | 151 |
| NSPTM_S | TTYFN | | FS | | WT | |
| CD3_sp11a_VHVL_TT_Y | RSSQSLVRSD | 149 | RVSNR | 150 | LQSSHFP | 151 |
| | GTTYFN | | FS | | WT | |
| CD3_sp11a_VHVL_TT_S | RSSQSLVRSD | 149 | RVSNR | 150 | LQSSHFP | 151 |
| | GTTYFN | | FS | | WT | |
| CD3_sp11a_VHVL_TT_SA | RSSQSLVRSEG | 487 | RVSNR | 150 | LQSSHFP | 151 |
| NSPTM | TTYFN | | FS | | WT | |
| CD3_SP11AVH3_VLK_3_Y | RSSQSLVRSEG | 487 | RVSNR | 150 | LQSSHFP | 151 |
| | TTYFN | | FS | | WT | |
| CD3_SP11AVH3_VLK_3_S | RSSQSLVRSEG | 487 | RVSNR | 150 | LQSSHFP | 151 |
| | TTYFN | | FS | | WT | |
| CD3_SP11AVH3_VLK_3_Y | RSSQSLVRSD | 149 | RVSNR | 150 | LQSSHFP | 151 |
| PTM | GTTYFN | | FS | | WT | |
| CD3_SP11AVH3_VLK_3_S_ | RSSQSLVRSD | 149 | RVSNR | 150 | LQSSHFP | 151 |
| PTM | GTTYFN | | FS | | WT | |
| CD3 SP11AVH3_VLK_3_Y_ | RSSQSLVRSEG | 487 | RVSNR | 150 | LQSSHFP | 151 |
| SW | TTYFN | | FS | | WT | |
| CD3 SP11AVH3_VLK_3_S_ | RSSQSLVRSEG | 487 | RVSNR | 150 | LQSSHFP | 151 |
| SW | TTYFN | | FS | | WT | |
| CD3_SP11AVH3_VLK_3_Y_ | RSSQSLVRSD | 149 | RVSNR | 150 | LQSSHFP | 151 |
| PTM_SW | GTTYFN | | FS | | WT | |
| CD3_SP11AVH3_VLK_3_S_ | RSSQSLVRSD | 149 | RVSNR | 150 | LQSSHFP | 151 |
| TM | GTTYFN | | FS | | WT | |
| CD3_SP11AVH3_VLK_ | RSSQSLVRSD | 149 | RVSNR | 150 | LQSSHFP | 151 |
| SWPTM | GTTYFN | | FS | | WT | |
| CD3_SP11AVH3_VLK_3_SW | RSSQSLVRSEG | 487 | RVSNR | 150 | LQSSHFP | 151 |
| | TTYFN | | FS | | WT | |
| CD3_sp11a_VH1_VK2_Y | RSSQSLVRSD | 149 | RVSNR | 150 | LQSSHFP | 151 |
| | GTTYFN | | FS | | WT | |
| CD3_sp11a_VH1_VK2_S | RSSQSLVRSD | 149 | RVSNR | 150 | LQSSHFP | 151 |
| | GTTYFN | | FS | | WT | |
| CD3_sp11a_VH1_VK2_Y_ | RSSQSLVRSEG | 487 | RVSNR | 150 | LQSSHFP | 151 |
| PTM | TTYFN | | FS | | WT | |
| CD3_sp11a_VH1_VK2_S_ | RSSQSLVRSEG | 487 | RVSNR | 150 | LQSSHFP | 151 |
| PTM | TTYFN | | FS | | WT | |
| CD3_sp11a_VH1_VK2_Y_ | RSSQSLVRSD | 149 | RVSNR | 150 | LQSSHFP | 151 |
| SW | GTTYFN | | FS | | WT | |
| CD3_sp11a_VH1_VK2_S_ | RSSQSLVRSD | 149 | RVSNR | 150 | LQSSHFP | 151 |
| SW | GTTYFN | | FS | | WT | |
| CD3_sp11a_VH1_VK2_Y_ | RSSQSLVRSEG | 487 | RVSNR | 150 | LQSSHFP | 151 |
| PTM | TTYFN | | FS | | WT | |
| CD3_sp11a_VH1_VK2_S_ | RSSQSLVRSEG | 487 | RVSNR | 150 | LQSSHFP | 151 |
| PTM_SW | TTYFN | | FS | | WT | |
| CD3_sp11a_VH1_VK2_SW | RSSQSLVRSD | 149 | RVSNR | 150 | LQSSHFP | 151 |
| | GTTYFN | | FS | | WT | |
| CD3_sp11a_VH1_VK2_SW_ | RSSQSLVRSEG | 487 | RVSNR | 150 | LQSSHFP | 151 |
| PTM | TTYFN | | FS | | WT | |
| CD3_SP11A_VH3_VLK1_Y | RSSQSLVRSEG | 487 | RVSNR | 150 | LQSSHFP | 151 |
| | TTYFN | | FS | | WT | |
| CD3_SP11A_VH3_VLK1_S | RSSQSLVRSEG | 487 | RVSNR | 150 | LQSSHFP | 151 |
| | TTYFN | | FS | | WT | |
| CD3_SP11A_VH3_VLK1_Y_ | RSSQSLVRSD | 149 | RVSNR | 150 | LQSSHFP | 151 |
| PTM | GTTYFN | | FS | | WT | |
| CD3_SP11A_VH3_VLK1_S_ | RSSQSLVRSD | 149 | RVSNR | 150 | LQSSHFP | 151 |
| PTM | GTTYFN | | FS | | WT | |
| CD3_SP11A_VH3_VLK1_Y_ | RSSQSLVRSEG | 487 | RVSNR | 150 | LQSSHFP | 151 |
| SW | TTYFN | | FS | | WT | |
| CD3_SP11A_VH3_VLK1_S_ | RSSQSLVRSEG | 487 | RVSNR | 150 | LQSSHFP | 151 |
| SW | TTYFN | | FS | | WT | |
| CD3_SP11A_VH3_VLK1_Y_ | RSSQSLVRSD | 149 | RVSNR | 150 | LQSSHFP | 151 |
| PTM | GTTYFN | | FS | | WT | |
| CD3_SP11A_VH3_VLK1_S_ | RSSQSLVRSD | 149 | RVSNR | 150 | LQSSHFP | 151 |
| PTM_SW | GTTYFN | | FS | | WT | |
| CD3_SP11A_VH3_VLK1P | RSSQSLVRSD | 149 | RVSNR | 150 | LQSSHFP | 151 |
| TM_SW | GTTYFN | | FS | | WT | |
| CD3_SP11A_VH3_VLK1_S | RSSQSLVRSEG | 487 | RVSNR | 150 | LQSSHFP | 151 |
| W | TTYFN | FS | WT | | | |
| CD3_SP11A_VH5_VK2_Y | RSSQSLVRSD | 149 | RVSNR | 150 | LQSSHFP | 151 |
| | GTTYFN | | FS | | WT | |
| CD3_SP11A_VH5_VK2_S | RSSQSLVRSD | 149 | RVSNR | 150 | LQSSHFP | 151 |
| | GTTYFN | | FS | | WT | |
| CD3_SP11A_VH5_VK2_Y_ | RSSQSLVRSD | 149 | RVSNR | 150 | LQSSHFP | 151 |
| PTM | GTTYFN | | FS | | WT | |
| CD3_SP11A_VH5_VK2_S_ | RSSQSLVRSD | 149 | RVSNR | 150 | LQSSHFP | 151 |
| PTM | GTTYFN | | FS | | WT | |
| CD3_SP11A_VH5_VK2_Y_ | RSSQSLVRSD | 149 | RVSNR | 150 | LQSSHFP | 151 |
| SW | GTTYFN | | FS | | WT | |
| CD3_SP11A_VH5_VK2_S_ | RSSQSLVRSD | 149 | RVSNR | 150 | LQSSHFP | 151 |
| SW | GTTYFN | | FS | | WT | |
| CD3_SP11A_VH5_VK2_Y_ | RSSQSLVRSD | 149 | RVSNR | 150 | LQSSHFP | 151 |
| PTM_SW | GTTYFN | | FS | | WT | |
| CD3_SP11A_VH5_VK2_S_ | RSSQSLVRSD | 149 | RVSNR | 150 | LQSSHFP | 151 |
| PTM_SW | GTTYFN | | FS | | WT | |
| CD3_5P11A_VH5_VK2_P | RSSQSLVRSD | 149 | RVSNR | 150 | LQSSHFP | 151 |
| TM_SW | GTTYFN | | FS | | WT | |
| CD3_SP11A_VH5_VK2_S | RSSQSLVRSD | 149 | RVSNR | 150 | LQSSHFP | 151 |
| W | GTTYFN | | FS | | WT | |

TABLE 1E-1
CD3 Binders - Heavy Chain CDR sequences according to Chothia numbering
scheme

| | | SEQ | | SEQ ID | | SEQ ID |
|---|---|---|---|---|---|---|
| Binder | CDR-H1 | ID NO: | CDR-H2 | NO: | CDR-H3 | NO: |
| NOV292 | GFTFSKN | 139 | YYDSSK | 140 | FVWVDLDFDH | 141 |
| NOV123 | GYTFTSY | 171 | YPGHDA | 172 | PNTMMAPLAY | 173 |
| Sp10b | GYTFTSY | 171 | YPGHDA | 172 | PNTMMAPLAY | 167 |
| NOV453 | GFSLTTY | 203 | RYSGD | 204 | DPMYIPNYSYG | 205 |
| | | | | | VMNA | |
| NOV229 | GFSLTTY | 235 | RYSGD | 236 | DPMYIPNYSYG | 237 |
| | | | | | VMNA | |
| NOV110 | GYTFTSY | 267 | YPANGG | 268 | PVTMMAPLVF | 269 |
| NOV832 | GYTFTSY | 299 | YPANGG | 300 | PVTMMAPLVF | 301 |
| NOV589 | GFTFSKN | 331 | YYDSSR | 332 | FVWVDLDFDY | 333 |
| NOV580 | GFSLTTY | 363 | RYSGD | 364 | DPMYIPGYSYG | 365 |
| | | | | | VMNA | |
| NOV567 | GFAFRKY | 395 | YYDSSK | 396 | LNSEYD | 397 |
| NOV221 | GFSLTTY | 427 | RYSGD | 428 | DPMYIPGYSYG | 429 |
| | | | | | VMNA | |
| CD3_sp11a_bkm1 | GFTFSKN | 139 | YYDSSK | 140 | FVWVDLDFDH | 135 |
| CD3_SP11a_bkm2 | GFTFSKN | 139 | YYDSSK | 140 | FVWVDLDFDH | 135 |
| CD3_sp11a_hz0 | GFTFSKN | 139 | YYDSSK | 140 | FVWVDLDFDH | 135 |
| CD3_SP11A_HZ1 | GFTFSKN | 139 | YYDSSK | 140 | FVWVDLDFDH | 135 |
| CD3_sp11a_sansPTM_hz1 | GFTFSKQ | 479 | YYDSSK | 140 | FVWVDLDFDH | 135 |
| CD3_sp11a_sansPTM_rat | GFTFSKQ | 479 | YYDSSK | 140 | FVWVDLDFDH | 135 |
| CD3_sp11a_VHVL_YY | GFTFSKN | 139 | YYDSSK | 140 | FYYDLDFDH | 478 |
| CD3_SP11A_VHVL_SS | GFTFSKN | 139 | YYDSSK | 140 | FSSDLDFDH | 472 |
| CD3_SP11A_VHVL_WS | GFTFSKN | 139 | YYDSSK | 140 | FWSDLDFDH | 476 |
| CD3_sp11a_VHVL_SW | GFTFSKN | 139 | YYDSSK | 140 | FSVVDLDFDH | 473 |
| CD3_SP11A_VHVL_TT | GFTFSKN | 139 | YYDSSK | 140 | FTTDLDFDH | 474 |
| CD3_SP11A_VHVL_TW | GFTFSKN | 139 | YYDSSK | 140 | FTWDLDFDH | 475 |
| CD3_SP11A_VHVL_VVT | GFTFSKN | 139 | YYDSSK | 140 | FVVTDLDFDH | 477 |
| CD3_SP11A_VH3_VLK_3 | GFTFSKN | 139 | YYDSSK | 140 | FVWVDLDFDH | 135 |
| CD3_sp11a_VH1_VK2 | GFTFSKQ | 479 | YYDSSK | 140 | FVWVDLDFDH | 135 |
| CD3_SP11A_VH3_VLK1 | GFTFSKN | 139 | YYDSSK | 140 | FVWVDLDFDH | 135 |
| CD3_SP11A_VH5_VK2 | GFTFSKQ | 479 | YYDSSK | 140 | FVWVDLDFDH | 135 |
| CD3_sp9aFW1_VL_VH_S5 | GFSLTTY | 203 | RYSGD | 204 | DPMYIPNYAYG | 471 |
| 6G | | | | | VMNA | |
| CD3_SP9AFW4_VL_VH_S | GFSLTTY | 203 | RYSGD | 204 | DPMYIPNYAYG | 471 |
| 56G | | | | | VMNA | |
| CD3_sp9aFW1_VLVH | GFSLTTY | 203 | RYSGD | 204 | DPMYIPNYAYG | 471 |
| | | | | | VMNA | |
| CD3_sp9aFW4_VLVH | GFSLTTY | 203 | RYSGD | 204 | DPMYIPNYAYG | 471 |
| | | | | | VMNA | |
| CD3_sp9arabtor_VHVL | GFSLTTY | 203 | RYSGD | 204 | DPMYIPNYAYG | 471 |
| | | | | | VMNA | |
| CD3_sp9arabtor_VLVH | GFSLTTY | 203 | RYSGD | 204 | DPMYIPNYAYG | 471 |
| | | | | | VMNA | |
| CD3_sp11a_VHVL_YY_SA | GFTFSKQ | 479 | YYDSSK | 140 | FYYDLDFDH | 478 |
| NSPTM | | | | | | |
| CD3_sp11a_VHVL_YY_SA | GFTFSKQ | 479 | YYDSSK | 140 | YYYDLDFDH | 627 |
| NSPTM_Y | | | | | | |
| CD3_sp11a_VHVL_YY_SA | GFTFSKQ | 479 | YYDSSK | 140 | SYYDLDFDH | 619 |
| NSPTM_S | | | | | | |
| CD3_sp11a_VHVL_YY_Y | GFTFSKN | 139 | YYDSSK | 140 | YYYDLDFDH | 627 |
| CD3_sp11a_VHVL_YY_s | GFTFSKN | 139 | YYDSSK | 140 | SYYDLDFDH | 619 |
| CD3_sp11a_VHVL_SS_SA | GFTFSKQ | 479 | YYDSSK | 140 | FSSDLDFDH | 472 |
| NSPTM | | | | | | |
| CD3_sp11a_VHVL_SS_SA | GFTFSKQ | 479 | YYDSSK | 140 | YSSDLDFDH | 620 |
| NSPTM_Y | | | | | | |
| CD3_sp11a_VHVL_SS_SA | GFTFSKQ | 479 | YYDSSK | 140 | SSSDLDFDH | 613 |
| NSPTM_S | | | | | | |
| CD3_sp11a_VHVL_SS_Y | GFTFSKN | 139 | YYDSSK | 140 | YSSDLDFDH | 620 |
| CD3_sp11a_VHVL_SS_S | GFTFSKN | 139 | YYDSSK | 140 | SSSDLDFDH | 613 |
| CD3_sp11a_VHVL_SS_ | GFTFSKQ | 479 | YYDSSK | 140 | FSSDLDFDH | 472 |
| SANSPTM | | | | | | |
| CD3_sp11a_VHVL_WS_ | GFTFSKQ | 479 | YYDSSK | 140 | YWSDLDFDH | 624 |
| SANSPTM_Y | | | | | | |
| CD3_sp11a_VHVL_WS_ | GFTFSKQ | 479 | YYDSSK | 140 | SWSDLDFDH | 617 |
| SANSPTM_S | | | | | | |
| CD3_sp11a_VHVL_WS_Y | GFTFSKN | 139 | YYDSSK | 140 | YWSDLDFDH | 625 |
| CD3_sp11a_VHVL_WS_S | GFTFSKN | 139 | YYDSSK | 140 | SWSDLDFDH | 617 |
| CD3_sp11a_VHVL_WS_ | GFTFSKQ | 479 | YYDSSK | 140 | FWSDLDFDH | 476 |
| SANSPTM | | | | | | |
| CD3_sp11a_VHVL_SW_ | GFTFSKQ | 479 | YYDSSK | 140 | YSVVDLDFDH | 621 |
| SANSPTM_Y | | | | | | |
| CD3_sp11a_VHVL_SW_ | GFTFSKQ | 479 | YYDSSK | 140 | SSVVDLDFDH | 614 |
| SANSPTM_S | | | | | | |
| CD3_sp11a_VHVL_SW_Y | GFTFSKN | 139 | YYDSSK | 140 | YSVVDLDFDH | 621 |
| CD3_sp11a_VHVL_SW_S | GFTFSKN | 139 | YYDSSK | 140 | SSVVDLDFDH | 614 |
| CD3_sp11a_VHVL_SW_ | GFTFSKQ | 479 | YYDSSK | 140 | FSVVDLDFDH | 473 |
| SANSPTM | | | | | | |
| CD3_sp11a_VHVL_TW_ | GFTFSKQ | 479 | YYDSSK | 140 | YTVVDLDFDH | 623 |
| SANSPTM_Y | | | | | | |
| CD3_sp11a_VHVL_TW_ | GFTFSKQ | 479 | YYDSSK | 140 | STVVDLDFDH | 616 |
| SANSPTM_S | | | | | | |
| CD3_sp11a_VHVL_TW_Y | GFTFSKN | 139 | YYDSSK | 140 | YTWDLDFDH | 623 |
| CD3_sp11a_VHVL_TW_S | GFTFSKN | 139 | YYDSSK | 140 | STWDLDFDH | 616 |
| CD3_sp11a_VHVL_TW_ | GFTFSKQ | 479 | YYDSSK | 140 | FTVVDLDFDH | 475 |
| SANSPTM | | | | | | |
| CD3_sp11a_VHVL_TT_ | GFTFSKQ | 479 | YYDSSK | 140 | YTTDLDFDH | 622 |
| SANSPTM_Y | | | | | | |
| CD3_sp11a_VHVL_TT_SA | GFTFSKQ | 479 | YYDSSK | 140 | STTDLDFDH | 615 |
| NSPTM_S | | | | | | |
| CD3_sp11a_VHVL_TT_Y | GFTFSKN | 139 | YYDSSK | 140 | YTTDLDFDH | 622 |
| CD3_sp11a_VHVL_TT_S | GFTFSKN | 139 | YYDSSK | 140 | STTDLDFDH | 615 |
| CD3_sp11a_VHVL_TT_SA | GFTFSKQ | 479 | YYDSSK | 140 | FTTDLDFDH | 474 |
| NSPTM | | | | | | |
| CD3_SP11AVH3_VLK_3_Y | GFTFSKN | 139 | YYDSSK | 140 | YVWVDLDFDH | 626 |
| CD3_SP11AVH3_VLK_3_S | GFTFSKN | 139 | YYDSSK | 140 | SVWVDLDFDH | 618 |
| CD3_SP11AVH3_VLK_3_Y_ | GFTFSKN | 139 | YYDSSK | 140 | YVWVDLDFDH | 626 |
| PTM | | | | | | |
| CD3_SP11AVH3_VLK_3_ | GFTFSKN | 139 | YYDSSK | 140 | SVWVDLDFDH | 618 |
| S_PTM | | | | | | |
| CD3_SP11AVH3_VLK_3_ | GFTFSKN | 139 | YYDSSK | 140 | YSVVDLDFDH | 621 |
| Y_SW | | | | | | |
| CD3_SP11AVH3_VLK_3_ | GFTFSKN | 139 | YYDSSK | 140 | SSVVDLDFDH | 614 |
| S_SW | | | | | | |
| CD3_SP11AVH3_VLK_3_ | GFTFSKN | 139 | YYDSSK | 140 | YSVVDLDFDH | 621 |
| Y_PTM_SW | | | | | | |
| CD3_SP11AVH3_VLK_3_ | GFTFSKN | 139 | YYDSSK | 140 | SSVVDLDFDH | 614 |
| S_SWPTM | | | | | | |
| CD3_SP11AVH3_VLK_SW | GFTFSKN | 139 | YYDSSK | 140 | FSVVDLDFDH | 473 |
| PTM | | | | | | |
| CD3_SP11AVH3_VLK_3_ | GFTFSKN | 139 | YYDSSK | 140 | FSVVDLDFDH | 473 |
| SW | | | | | | |
| CD3_sp11a_VH1_VK2_Y | GFTFSKQ | 479 | YYDSSK | 140 | YVWVDLDFDH | 626 |
| CD3_sp11a_VH1_VK2_S | GFTFSKQ | 479 | YYDSSK | 140 | SVWVDLDFDH | 618 |
| CD3_sp11a_VH1_VK2_Y_ | GFTFSKN | 139 | YYDSSK | 140 | YVWVDLDFDH | 626 |
| PTM | | | | | | |
| CD3_sp11a_VH1_VK2_S_ | GFTFSKN | 139 | YYDSSK | 140 | SVWVDLDFDH | 618 |
| PTM | | | | | | |
| CD3_sp11a_VH1_VK2_Y_ | GFTFSKQ | 479 | YYDSSK | 140 | YSVVDLDFDH | 621 |
| SW | | | | | | |
| CD3_sp11a_VH1_VK2_S_ | GFTFSKQ | 479 | YYDSSK | 140 | SSVVDLDFDH | 614 |
| SW | | | | | | |
| CD3_sp11a_VH1_VK2_Y_ | GFTFSKN | 139 | YYDSSK | 140 | YSVVDLDFDH | 621 |
| PTM | | | | | | |
| CD3_sp11a_VH1_VK2_S_ | GFTFSKN | 139 | YYDSSK | 140 | SSVVDLDFDH | 614 |
| PTM_SW | | | | | | |
| CD3_sp11a_VH1_VK2_SW | GFTFSKQ | 479 | YYDSSK | 140 | FSVVDLDFDH | 473 |
| CD3_sp11a_VH1_VK2_S | GFTFSKN | 139 | YYDSSK | 140 | FSVVDLDFDH | 473 |
| W_PTM | | | | | | |
| CD3_SP11A_VH3_VLK1_Y | GFTFSKN | 139 | YYDSSK | 140 | YVWVDLDFDH | 626 |
| CD3_SP11A_VH3_VLK1_S | GFTFSKN | 139 | YYDSSK | 140 | SVWVDLDFDH | 618 |
| CD3_SP11A_VH3_VLK1_ | GFTFSKQ | 479 | YYDSSK | 140 | YVWVDLDFDH | 626 |
| Y_PTM | | | | | | |
| CD3_SP11A_VH3_VLK1_ | GFTFSKN | 139 | YYDSSK | 140 | SVWVDLDFDH | 618 |
| S_PTM | | | | | | |
| CD3_SP11A_BH3_VLK1_ | GFTSKN | 139 | YYDSSK | 140 | YSWDLDFDH | 621 |
| Y_SW | | | | | | |
| CD3_SP11A_VH3_VLK1_ | GFTSKN | 139 | YYDSSK | 140 | SSWDLDFDH | 614 |
| CD3_SP11A_VH3_VLK1_ | GFTFSKQ | 479 | YYDSSK | 140 | YVWVDLDFDH | 626 |
| Y_PTM | | | | | | |
| CD3_SP11A_VH3_VLK1_ | GFTFSKN | 139 | YYDSSK | 140 | SSVVDLDFDH | 614 |
| S_PTM_SW | | | | | | |
| CD3_SP11A_VH3_VLK1_ | GFTFSKN | 139 | YYDSSK | 140 | SSWDLDFDH | 473 |
| S_PTM_SW | | | | | | |
| CD3_SP11A_VH3_VLK1_ | GFTFSKN | 139 | YYDSSK | 140 | FSWDLDFDH | 473 |
| SW | | | | | | |
| CD3_SP11A_VH5_VK2_Y | GFTFSKQ | 479 | YYDSSK | 140 | YVWVDLDFDH | 626 |
| CD3_SP11A_VH5_VK2_S | GFTFSKQ | 479 | YYDSSK | 140 | SVWVDLDFDH | 618 |
| CD3_SP11A_VH5_VK2_Y_ | GFTFSKN | 139 | YYDSSK | 140 | YVWVDLDFDH | 626 |
| PTIV1 | | | | | | |
| CD3_SP11A_VH5_VK2_S_ | GFTFSKN | 139 | YYDSSK | 140 | SVWVDLDFDH | 618 |
| PTM | | | | | | |
| CD3_SP11A_VH5_VK2_Y_ | GFTFSKQ | 479 | YYDSSK | 140 | YSVVDLDFDH | 621 |
| SW | | | | | | |
| CD3_SP11A_VH5_VK2_S_ | GFTFSKQ | 479 | YYDSSK | 140 | SSVVDLDFDH | 614 |
| SW | | | | | | |
| CD3_SP11A_VH5_VK2_Y_ | GFTFSKN | 139 | YYDSSK | 140 | YSVVDLDFDH | 621 |
| SW | | | | | | |
| CD3_SP11A_VH5_VK2_S_ | GFTFSKN | 139 | YYDSSK | 140 | SSVVDLDFDH | 614 |
| PTM_SW | | | | | | |
| CD3_SP11A_VH5_VK2_ | GFTFSKN | 139 | YYDSSK | 140 | FSVVDLDFDH | 473 |
| PTM_SW | | | | | | |
| CD3_SP11A_VH5_VK2_SW | GFTFSKQ | 479 | YYDSSK | 140 | FSVVDLDFDH | 473 |

TABLE 1E-2
CD3 Binders - Light Chain CDR sequences according to Chothia numbering scheme

| | | SEQ ID | | SEQ ID | | SEQ ID |
|---|---|---|---|---|---|---|
| Binder | CDR-L1 | NO: | CDR-L2 | NO: | CDR-L3 | NO: |
| NOV292 | SQSLVRSD | 155 | RVS | 156 | SSHFPW | 157 |
| | GTTY | | | | | |
| NOV123 | SQSLIYSIGN | 187 | RVS | 188 | STHLPY | 189 |
| | TY | | | | | |
| Sp10b | SQSLIYSIGN | 187 | RVS | 156 | STHLPY | 189 |
| | TY | | | | | |
| NOV453 | SQNINNY | 219 | NTD | 220 | HRSRY | 221 |
| NOV229 | SQNINNY | 251 | NTD | 252 | HRSRY | 253 |
| NOV110 | SQSLVYSH | 283 | RVS | 284 | STHLPY | 285 |
| | GNTY | | | | | |
| NOV832 | SQSLVYSH | 315 | RVS | 316 | STHLPY | 317 |
| | GNTY | | | | | |
| NOV589 | SQSLVRSD | 347 | RVS | 348 | SSHFPW | 349 |
| | GTTY | | | | | |
| NOV580 | SQNIDKY | 379 | NTN | 380 | HRSSY | 381 |
| NOV567 | SQSIGNS | 411 | STS | 412 | YATYPY | 413 |
| NOV221 | SQNIDKY | 443 | NTN | 444 | HRSGY | 445 |
| CD3_sp11a_bkm1 | SQSLVRSD | 155 | RVS | 156 | SSHFPW | 157 |
| | GTTY | | | | | |
| CD3_SP11a_bkm2 | SQSLVRSD | 155 | RVS | 156 | SSHFPW | 157 |
| | GTTY | | | | | |
| CD3_sp11a_hz0 | SQSLVRSD | 155 | RVS | 156 | SSHFPW | 157 |
| | GTTY | | | | | |
| CD3_SP11A_HZ1 | SQSLVRSD | 155 | RVS | 156 | SSHFPW | 157 |
| | GTTY | | | | | |
| CD3_sp11a_sansPTM_hz1 | SQSLVRSE | 488 | RVS | 156 | SSHFPW | 157 |
| | GTTY | | | | | |
| CD3_sp11a_sansPTM_rat | SQSLVRSE | 488 | RVS | 156 | SSHFPW | 157 |
| | GTTY | | | | | |
| CD3_sp11a_VHVL_YY | SQSLVRSD | 155 | RVS | 156 | SSHFPW | 157 |
| | GTTY | | | | | |
| CD3_SP11A_VHVL_SS | SQSLVRSD | 155 | RVS | 156 | SSHFPW | 157 |
| | GTTY | | | | | |
| CD3_SP11A_VHVL_WS | SQSLVRSD | 155 | RVS | 156 | SSHFPW | 157 |
| | GTTY | | | | | |
| CD3_sp11a_VHVL_SW | SQSLVRSD | 155 | RVS | 156 | SSHFPW | 157 |
| | GTTY | | | | | |
| CD3_SP11A_VHVL_TT | SQSLVRSD | 155 | RVS | 156 | SSHFPW | 157 |
| | GTTY | | | | | |
| CD3_SP11A_VHVL_TW | SQSLVRSD | 155 | RVS | 156 | SSHFPW | 157 |
| | GTTY | | | | | |
| CD3_SP11A_VHVL_VVT | SQSLVRSD | 155 | RVS | 156 | SSHFPW | 157 |
| | GTTY | | | | | |
| CD3_SP11A VH3_VLK_3 | SQSLVRSE | 488 | RVS | 156 | SSHFPW | 157 |
| | GTTY | | | | | |
| CD3_sp11a_VH1_VK2 | SQSLVRSD | 155 | RVS | 156 | SSHFPW | 157 |
| | GTTY | | | | | |
| CD3_SP11A_VH3_VLK1 | SQSLVRSE | 488 | RVS | 156 | SSHFPW | 157 |
| | GTTY | | | | | |
| CD3_SP11A_VH5_VK2 | SQSLVRSD | 155 | RVS | 156 | SSHFPW | 157 |
| | GTTY | | | | | |
| CD3_sp9aFW1_VL_VH_S5 | SQNINNY | 219 | NTD | 220 | HRSRY | 221 |
| 6G | | | | | | |
| CD3_SP9AFW4_VL_VH_S | SQNINNY | 219 | NTD | 220 | HRSRY | 221 |
| 56G | | | | | | |
| CD3_sp9aFW1_VLVH | SQNINNY | 219 | NTD | 220 | HRSRY | 221 |
| CD3_sp9aFW4_VLVH | SQNINNY | 219 | NTD | 220 | HRSRY | 221 |
| CD3_sp9arabtor_VHVL | SQNINNY | 219 | NTD | 220 | HRSRY | 221 |
| CD3_sp9arabtor_VLVH | SQNINNY | 219 | NTD | 220 | HRSRY | 221 |
| CD3_sp11a_VHVL_YY_SA | SQSLVRSE | 488 | RVS | 156 | SSHFPW | 157 |
| NSPTM | GTTY | | | | | |
| CD3_sp11a_VHVL_YY_SA | SQSLVRSE | 488 | RVS | 156 | SSHFPW | 157 |
| NSPTM_Y | GTTY | | | | | |
| CD3_sp11a_VHVL_YY_SA | SQSLVRSE | 488 | RVS | 156 | SSHFPW | 157 |
| NSPTM_S | GTTY | | | | | |
| CD3_sp11a_VHVL_YY_Y | SQSLVRSD | 155 | RVS | 156 | SSHFPW | 157 |
| | GTTY | | | | | |
| CD3_sp11a_VHVL_YY_s | SQSLVRSD | 155 | RVS | 156 | SSHFPW | 157 |
| | GTTY | | | | | |
| CD3_sp11a_VHVL_SS_SA | SQSLVRSE | 488 | RVS | 156 | SSHFPW | 157 |
| NSPTM | GTTY | | | | | |
| CD3_sp11a_VHVL_SS_SA | SQSLVRSE | 488 | RVS | 156 | SSHFPW | 157 |
| NSPTM_Y | GTTY | | | | | |
| CD3_sp11a_VHVL_SS_SA | SQSLVRSE | 488 | RVS | 156 | SSHFPW | 157 |
| NSPTM_S | GTTY | | | | | |
| CD3_sp11a_VHVL_SS_Y | SQSLVRSD | 155 | RVS | 156 | SSHFPW | 157 |
| | GTTY | | | | | |
| CD3_sp11a_VHVL_SS_S | SQSLVRSD | 155 | RVS | 156 | SSHFPW | 157 |
| | GTTY | | | | | |
| CD3_sp11a_VHVL_SS_ | SQSLVRSE | 488 | RVS | 156 | SSHFPW | 157 |
| SANSPTM | GTTY | | | | | |
| CD3_sp11a_VHVL_WS_ | SQSLVRSE | 488 | RVS | 156 | SSHFPW | 157 |
| SANSPTM_Y | GTTY | | | | | |
| CD3_sp11a_VHVL_WS_ | SQSLVRSE | 488 | RVS | 156 | SSHFPW | 157 |
| SANSPTM_S | GTTY | | | | | |
| CD3_sp11a_VHVL_WS_Y | SQSLVRSD | 155 | RVS | 156 | SSHFPW | 157 |
| | GTTY | | | | | |
| CD3_sp11a_VHVL_WS_S | SQSLVRSD | 155 | RVS | 156 | SSHFPW | 157 |
| | GTTY | | | | | |
| CD3_sp11a_VHVL_WS_ | SQSLVRSE | 488 | RVS | 156 | SSHFPW | 157 |
| SANSPTM | GTTY | | | | | |
| CD3_sp11a_VHVL_SW_ | SQSLVRSE | 488 | RVS | 156 | SSHFPW | 157 |
| SANSPTM_Y | GTTY | | | | | |
| CD3_sp11a_VHVL_SW_ | SQSLVRSE | 488 | RVS | 156 | SSHFPW | 157 |
| SANSPTM_S | GTTY | | | | | |
| CD3_sp11a_VHVL_SW_Y | SQSLVRSD | 155 | RVS | 156 | SSHFPW | 157 |
| | GTTY | | | | | |
| CD3_sp11a_VHVL_SW_S | SQSLVRSD | 155 | RVS | 156 | SSHFPW | 157 |
| | GTTY | | | | | |
| CD3_sp11a_VHVL_SW_ | SQSLVRSE | 488 | RVS | 156 | SSHFPW | 157 |
| SANSPTM | GTTY | | | | | |
| CD3_sp11a_VHVL_TW_ | SQSLVRSE | 488 | RVS | 156 | SSHFPW | 157 |
| SANSPTM_Y | GTTY | | | | | |
| CD3_sp11a_VHVL_TW_ | SQSLVRSE | 488 | RVS | 156 | SSHFPW | 157 |
| SANSPTM_S | GTTY | | | | | |
| CD3_sp11a_VHVL_TW_Y | SQSLVRSD | 155 | RVS | 156 | SSHFPW | 157 |
| | GTTY | | | | | |
| CD3_sp11a_VHVL_TW_S | SQSLVRSD | 155 | RVS | 156 | SSHFPW | 157 |
| | GTTY | | | | | |
| CD3_sp11a_VHVL_TW_ | SQSLVRSE | 488 | RVS | 156 | SSHFPW | 157 |
| SANSPTM | GTTY | | | | | |
| CD3_sp11a_VHVL_TT_ | SQSLVRSE | 488 | RVS | 156 | SSHFPW | 157 |
| SANSPTM_Y | GTTY | | | | | |
| CD3_sp11a_VHVL_TT_SA | SQSLVRSE | 488 | RVS | 156 | SSHFPW | 157 |
| NSPTM_S | GTTY | | | | | |
| CD3_sp11a_VHVL_TT_Y | SQSLVRSD | 155 | RVS | 156 | SSHFPW | 157 |
| | GTTY | | | | | |
| CD3_sp11a_VHVL_TT_S | SQSLVRSD | 155 | RVS | 156 | SSHFPW | 157 |
| | GTTY | | | | | |
| CD3_sp11a_VHVL_TT_SA | SQSLVRSE | 488 | RVS | 156 | SSHFPW | 157 |
| NSPTM | GTTY | | | | | |
| CD3_SP11AVH3_VLK_3_Y | SQSLVRSE | 488 | RVS | 156 | SSHFPW | 157 |
| | GTTY | | | | | |
| CD3_SP11AVH3_VLK_3_S | SQSLVRSE | 488 | RVS | 156 | SSHFPW | 157 |
| | GTTY | | | | | |
| CD3_SP11AVH3_VLK_3_ | SQSLVRSD | 155 | RVS | 156 | SSHFPW | 157 |
| Y_PTM | GTTY | | | | | |
| CD3_SP11AVH3_VLK_3_ | SQSLVRSD | 155 | RVS | 156 | SSHFPW | 157 |
| S_PTM | GTTY | | | | | |
| CD3_SP11AVH3_VLK_3_ | SQSLVRSE | 488 | RVS | 156 | SSHFPW | 157 |
| Y_SW | GTTY | | | | | |
| CD3_SP11AVH3_VLK_3_ | SQSLVRSE | 488 | RVS | 156 | SSHFPW | 157 |
| S_SW | GTTY | | | | | |
| CD3_SP11AVH3_VLK_3_ | SQSLVRSD | 155 | RVS | 156 | SSHFPW | 157 |
| Y_PTM SW | GTTY | | | | | |
| CD3_SP11AVH3_VLK_3_ | SQSLVRSD | 155 | RVS | 156 | SSHFPW | 157 |
| S_SWPTM | GTTY | | | | | |
| CD3_SP11AVH3_VLK_SW | SQSLVRSD | 155 | RVS | 156 | SSHFPW | 157 |
| PTM | GTTY | | | | | |
| CD3_SP11AVH3_VLK_3_SW | SQSLVRSE | 488 | RVS | 156 | SSHFPW | 157 |
| | GTTY | | | | | |
| CD3_sp11a_VH1_VK2_Y | SQSLVRSD | 155 | RVS | 156 | SSHFPW | 157 |
| | GTTY | | | | | |
| CD3_sp11a_VH1_VK2_S | SQSLVRSD | 155 | RVS | 156 | SSHFPW | 157 |
| | GTTY | | | | | |
| CD3_sp11a_VH1_VK2_Y_ | SQSLVRSE | 488 | RVS | 156 | SSHFPW | 157 |
| PTM | GTTY | | | | | |
| CD3_sp11a_VH1_VK2_S_ | SQSLVRSD | 155 | RVS | 156 | SSHFPW | 157 |
| PTM | GTTY | | | | | |
| CD3_sp11a_VH1_VK2_Y_ | SQSLVRSD | 155 | RVS | 156 | SSHFPW | 157 |
| SW | GTTY | | | | | |
| CD3_sp11a_VH1_VK2_S_ | SQSLVRSD | 155 | RVS | 156 | SSHFPW | 157 |
| SW | GTTY | | | | | |
| CD3_sp11a_VH1_VK2_Y_ | SQSLVRSE | 488 | RVS | 156 | SSHFPW | 157 |
| PTM | GTTY | | | | | |
| CD3_sp11a_VH1_VK2_S_ | SQSLVRSD | 155 | RVS | 156 | SSHFPW | 157 |
| PTM_SW | GTTY | | | | | |
| CD3_sp11a_VH1_VK2_SW | SQSLVRSD | 155 | RVS | 156 | SSHFPW | 157 |
| | GTTY | | | | | |
| CD3_sp11a_VH1_VK2_SW_ | SQSLVRSE | 488 | RVS | 156 | SSHFPW | 157 |
| PTM | GTTY | | | | | |
| CD3_SP11A_VH3_VLK1_Y | SQSLVRSE | 488 | RVS | 156 | SSHFPW | 157 |
| | GTTY | | | | | |
| CD3_SP11A_VH3_VLK1_S | SQSLVRSE | 488 | RVS | 156 | SSHFPW | 157 |
| | GTTY | | | | | |
| CD3_SP11A_VH3_VLK1_ | SQSLVRSD | 155 | RVS | 156 | SSHFPW | 157 |
| Y_PTM | GTTY | | | | | |
| CD3_SP11A_VH3_VLK1_ | SQSLVRSD | 155 | RVS | 156 | SSHFPW | 157 |
| S_PTM | GTTY | | | | | |
| CD3_SP11A_VH3_VLK1_ | SQSLVRSE | 488 | RVS | 156 | SSHFPW | 157 |
| Y_SW | GTTY | | | | | |
| CD3_SP11A_VH3_VLK1_ | SQSLVRSE | 488 | RVS | 156 | SSHFPW | 157 |
| S_SW | GTTY | | | | | |
| CD3_SP11A_VH3_VLK1_ | SQSLVRSD | 155 | RVS | 156 | SSHFPW | 157 |
| Y_PTM | GTTY | | | | | |
| CD3_SP11A_VH3_VLK1_ | SQSLVRSD | 155 | RVS | 156 | SSHFPW | 157 |
| S_PTM_SW | GTTY | | | | | |
| CD3_SP11A_VH3_VLK1P | SQSLVRSD | 155 | RVS | 156 | SSHFPW | 157 |
| TM_SW | GTTY | | | | | |
| CD3_SP11A_VH3_VLK1_ | SQSLVRSE | 488 | RVS | 156 | SSHFPW | 157 |
| SW | GTTY | | | | | |
| CD3_SP11A_VH5_VK2_Y | SQSLVRSD | 155 | RVS | 156 | SSHFPW | 157 |
| | GTTY | | | | | |
| CD3_SP11A_VH5_VK2_S | SQSLVRSD | 155 | RVS | 156 | SSHFPW | 157 |
| | GTTY | | | | | |
| CD3_SP11A_VH5_VK2_Y_ | SQSLVRSD | 155 | RVS | 156 | SSHFPW | 157 |
| PTM | GTTY | | | | | |
| CD3_SP11A_VH5_VK2_S_ | SQSLVRSD | 155 | RVS | 156 | SSHFPW | 157 |
| PTM | GTTY | | | | | |
| CD3_SP11A_VH5_VK2_Y_ | SQSLVRSD | 155 | RVS | 156 | SSHFPW | 157 |
| SW | GTTY | | | | | |
| CD3_SP11A_VH5_VK2_S_SW | SQSLVRSD | 155 | RVS | 156 | SSHFPW | 157 |
| | GTTY | | | | | |
| CD3_SP11A_VH5_VK2_Y_ | SQSLVRSD | 155 | RVS | 156 | SSHFPW | 157 |
| PTM_SW | GTTY | | | | | |
| CD3_SP11A_VH5_VK2_S_ | SQSLVRSD | 155 | RVS | 156 | SSHFPW | 157 |
| PTM_SW | GTTY | | | | | |
| CD3_SP11A_VH5_VK2_P | SQSLVRSD | 155 | RVS | 156 | SSHFPW | 157 |
| TM_SW | GTTY | | | | | |
| CD3_SP11A_VH5_VK2_SW | SQSLVRSD | 155 | RVS | 156 | SSHFPW | 157 |
| | GTTY | | | | | |

TABLE 1F-1
CD3 Binders - Heavy Chain CDR sequences according to IMGT numbering scheme

| | | SEQ ID | | SEQ ID | | SEQ ID |
|---|---|---|---|---|---|---|
| Binder | CDR-H1 | NO: | CDR-H2 | NO: | CDR-H3 | NO: |
| NOV292 | GFTFSKN | 142 | IYYDSSKM | 143 | ASFVWVDLDF | 144 |
| | G | | | | DH | |
| NOV123 | GYTFTSY | 174 | IYPGHDAI | 175 | VRPNTMMAP | 176 |
| | Y | | | | LAY | |
| Sp10b | GYTFTSY | 174 | IYPGHDAI | 175 | VRPNTMMAP | 176 |
| | Y | | | | LAY | |
| NOV453 | GFSLTTY | 206 | MRYSGDT | 207 | TSDPMYIPNY | 208 |
| | N | | | | SYGVMNA | |
| NOV229 | GFSLTTY | 238 | MRYSGDT | 239 | ARDPMYIPN | 240 |
| | N | | | | YSYGVMNA | |
| NOV110 | GYTFTSY | 270 | IYPANGGI | 271 | ARPVTMMAP | 272 |
| | Y | | | | LVF | |
| NOV832 | GYTFTSY | 302 | IYPANGGI | 303 | ARPVTMMAP | 304 |
| | Y | | | | LVF | |
| NOV589 | GFTFSKN | 334 | IYYDSSR | 335 | ASFVWVDLDF | 336 |
| | G | | M | | DY | |
| NOV580 | GFSLTTY | 366 | MRYSGDT | 367 | TRDPMYIPG | 368 |
| | N | | | | YSYGVMNA | |
| NOV567 | GFAFRKY | 398 | IYYDSSKM | 399 | AALNSEYD | 400 |
| | G | | | | | |
| NOV221 | GFSLTTY | 430 | MRYSGDT | 431 | TRDPMYIPG | 432 |
| | N | | | | YSYGVMNA | |
| CD3_sp11a_bkm1 | GFTFSKN | 142 | IYYDSSKM | 143 | ASFVWVDLDF | 144 |
| | G | | | | DH | |
| CD3_SP11a_bkm2 | GFTFSKN | 142 | IYYDSSKM | 143 | AKFVWVDLDF | 462 |
| | G | | | | DH | |
| CD3_sp11a_hz0 | GFTFSKN | 142 | IYYDSSKM | 143 | AKFVWVDLDF | 462 |
| | G | | | | DH | |
| CD3_SP11A_HZ1 | GFTFSKN | 142 | IYYDSSKM | 143 | ASFVWVDLDF | 144 |
| | G | | | | DH | |
| CD3_sp11a_sansPTM_hz1 | GFTFSKQ | 480 | IYYDSSKM | 143 | ASFVWVDLDF | 144 |
| | G | | | | DH | |
| CD3_sp11a_sansPTM_rat | GFTFSKQ | 480 | IYYDSSKM | 143 | ASFVWVDLDF | 144 |
| | G | | | | DH | |
| CD3_sp11a_VHVL_YY | GFTFSKN | 142 | IYYDSSKM | 143 | ASFYYDLDF | 470 |
| | G | | | | DH | |
| CD3_SP11A_VHVL_SS | GFTFSKN | 142 | IYYDSSKM | 143 | ASFSSDLDF | 464 |
| | G | | | | DH | |
| CD3_SP11A_VHVL_WS | GFTFSKN | 142 | IYYDSSKM | 143 | ASFWSDLDF | 468 |
| | G | | | | DH | |
| CD3_sp11a_VHVL_SW | GFTFSKN | 142 | IYYDSSKM | 143 | ASFSVVDLDF | 465 |
| | G | | | | DH | |
| CD3_SP11A_VHVL_TT | GFTFSKN | 142 | IYYDSSKM | 143 | ASFTTDLDFD | 466 |
| | G | | | | H | |
| CD3_SP11A_VHVL_TW | GFTFSKN | 142 | IYYDSSKM | 143 | ASFTWDLDF | 467 |
| | G | | | | DH | |
| CD3_SP11A_VHVL_VVT | GFTFSKN | 142 | IYYDSSKM | 143 | ASFVVTDLDF | 469 |
| | G | | | | DH | |
| CD3_SP11A VH3_VLK_3 | GFTFSKN | 142 | IYYDSSKM | 143 | ASFVWVDLDF | 144 |
| | G | | | | DH | |
| CD3_sp11a_VH1_VK2 | GFTFSKQ | 480 | IYYDSSKM | 143 | ASFVWVDLDF | 144 |
| | G | | | | DH | |
| CD3_SP11A_VH3_VLK1 | GFTFSKN | 142 | IYYDSSKM | 143 | ASFVWVDLDF | 144 |
| | G | | | | DH | |
| CD3_SP11A_VH5_VK2 | GFTFSKQ | 480 | IYYDSSKM | 143 | ASFVWVDLDF | 144 |
| | G | | | | DH | |
| CD3_sp9aFW1_VL_VH_S | GFSLTTY | 206 | MRYSGDT | 207 | ASDPMYIPNY | 463 |
| 56G | N | | | | AYGVMNA | |
| CD3_SP9AFW4_VL_VH_S | GFSLTTY | 206 | MRYSGDT | 207 | ASDPMYIPNY | 463 |
| 56G | N | | | | AYGVMNA | |
| CD3_sp9aFW1_VLVH | GFSLTTY | 206 | MRYSGDT | 207 | ASDPMYIPNY | 463 |
| | N | | | | AYGVMNA | |
| CD3_sp9aFW4_VLVH | GFSLTTY | 206 | MRYSGDT | 207 | ASDPMYIPNY | 463 |
| | N | | | | AYGVMNA | |
| CD3_sp9arabtor_VHVL | GFSLTTY | 206 | MRYSGDT | 207 | ASDPMYIPNY | 463 |
| | N | | | | AYGVMNA | |
| CD3_sp9arabtor_VLVH | GFSLTTY | 206 | MRYSGDT | 207 | ASDPMYIPNY | 463 |
| | N | | | | AYGVMNA | |
| CD3_sp11a_VHVL_YY_SA | GFTFSKQ | 480 | IYYDSSKM | 143 | ASFYYDLDF | 470 |
| NSPTM | G | | | | DH | |
| CD3_sp11a_VHVL_YY_SA | GFTFSKQ | 480 | IYYDSSKM | 143 | ASYYYDLDF | 554 |
| NSPTM_Y | G | | | | DH | |
| CD3_sp11a_VHVL_YY_SA | GFTFSKQ | 480 | IYYDSSKM | 143 | ASSYYDLDF | 547 |
| NSPTM_S | G | | | | DH | |
| CD3_sp11a_VHVL_YY_Y | GFTFSKN | 142 | IYYDSSKM | 143 | ASYYYDLDF | 554 |
| | G | | | | DH | |
| CD3_sp11a_VHVL_YY_s | GFTFSKN | 142 | IYYDSSKM | 143 | ASSYYDLDF | 547 |
| | G | | | | DH | |
| CD3_sp11a_VHVL_SS_SA | GFTFSKQ | 480 | IYYDSSKM | 143 | ASFSSDLDF | 464 |
| NSPTM | G | | | | DH | |
| CD3_sp11a_VHVL_SS_SA | GFTFSKQ | 480 | IYYDSSKM | 143 | ASYSSDLDF | 548 |
| NSPTM_Y | G | | | | DH | |
| CD3_sp11a_VHVL_SS_SA | GFTFSKQ | 480 | IYYDSSKM | 143 | ASSSSDLDF | 541 |
| NSPTM_S | G | | | | DH | |
| CD3_sp11a_VHVL_SS_Y | GFTFSKN | 142 | IYYDSSKM | 143 | ASYSSDLDF | 548 |
| | G | | | | DH | |
| CD3_sp11a_VHVL_SS_S | GFTFSKN | 142 | IYYDSSKM | 143 | ASSSSDLDF | 541 |
| | G | | | | DH | |
| CD3_sp11a_VHVL_SS_ | GFTFSKQ | 480 | IYYDSSKM | 143 | ASFSSDLDF | 464 |
| SANSPTM | G | | | | DH | |
| CD3_sp11a_VHVL_WS_ | GFTFSKQ | 480 | IYYDSSKM | 143 | ASYWSDLDF | 552 |
| SANSPTM_Y | G | | | | DH | |
| CD3_sp11a_VHVL_WS_ | GFTFSKQ | 480 | IYYDSSKM | 143 | ASSWSDLDF | 545 |
| SANSPTM_S | G | | | | DH | |
| CD3_sp11a_VHVL_WS_Y | GFTFSKN | 142 | IYYDSSKM | 143 | ASYWSDLDF | 552 |
| | G | | | | DH | |
| CD3_sp11a_VHVL_WS_S | GFTFSKN | 142 | IYYDSSKM | 143 | ASSWSDLDF | 545 |
| | G | | | | DH | |
| CD3_sp11a_VHVL_WS_ | GFTFSKQ | 480 | IYYDSSKM | 143 | ASFWSDLDF | 468 |
| SANSPTM | G | | | | DH | |
| CD3_sp11a_VHVL_SW_ | GFTFSKQ | 480 | IYYDSSKM | 143 | ASYSVVDLDF | 549 |
| SANSPTM_Y | G | | | | DH | |
| CD3_sp11a_VHVL_SW_ | GFTFSKQ | 480 | IYYDSSKM | 143 | ASSSVVDLDF | 542 |
| SANSPTM_S | G | | | | DH | |
| CD3_sp11a_VHVL_SW_Y | GFTFSKN | 142 | IYYDSSKM | 143 | ASYSVVDLDF | 549 |
| | G | | | | DH | |
| CD3_sp11a_VHVL_SW_S | GFTFSKN | 142 | IYYDSSKM | 143 | ASSSVVDLDF | 542 |
| | G | | | | DH | |
| CD3_sp11a_VHVL_SW_ | GFTFSKQ | 480 | IYYDSSKM | 143 | ASFSVVDLDF | 465 |
| SANSPTM | G | | | | DH | |
| CD3_sp11a_VHVL_TW_ | GFTFSKQ | 480 | IYYDSSKM | 143 | ASYTVVDLDF | 551 |
| SANSPTM_Y | G | | | | DH | |
| CD3_sp11a_VHVL_TW_ | GFTFSKQ | 480 | IYYDSSKM | 143 | ASSTVVDLDF | 544 |
| SANSPTM_S | G | | | | DH | |
| CD3_sp11a_VHVL_TW_ | GFTFSKN | 142 | IYYDSSKM | 143 | ASYTVVDLDF | 551 |
| Y | G | | | | DH | |
| CD3_sp11a_VHVL_TW_ | GFTFSKN | 142 | IYYDSSKM | 143 | ASSTVVDLDF | 544 |
| S | G | | | | DH | |
| CD3_sp11a_VHVL_TW_ | GFTFSKQ | 480 | IYYDSSKM | 143 | ASFTWDLDF | 467 |
| SANSPTM | G | | | | DH | |
| CD3_sp11a_VHVL_TT_ | GFTFSKQ | 480 | IYYDSSKM | 143 | ASYTTDLDFD | 550 |
| SANSPTM_Y | G | | | | H | |
| CD3_sp11a_VHVL_TT_SA | GFTFSKQ | 480 | IYYDSSKM | 143 | ASSTTDLDFD | 543 |
| NSPTM_S | G | | | | H | |
| CD3_sp11a_VHVL_TT_Y | GFTFSKN | 142 | IYYDSSKM | 143 | ASYTTDLDFD | 550 |
| | G | | | | H | |
| CD3_sp11a_VHVL_TT_S | GFTFSKN | 142 | IYYDSSKM | 143 | ASSTTDLDFD | 543 |
| | G | | | | H | |
| CD3_sp11a_VHVL_TT_SA | GFTFSKQ | 480 | IYYDSSKM | 143 | ASFTTDLDFD | 466 |
| NSPTM | G | | | | H | |
| CD3_SP11AVH3_VLK_3_ | GFTFSKN | 142 | IYYDSSKM | 143 | ASYVWVDLD | 553 |
| Y | G | | | | DH | |
| CD3_SP11AVH3_VLK_3_ | GFTFSKN | 142 | IYYDSSKM | 143 | ASSVWVDLDF | 546 |
| S | G | | | | DH | |
| CD3_SP11AVH3_VLK_3_ | GFTFSKN | 142 | IYYDSSKM | 143 | ASYVWVDLDF | 553 |
| Y_PTM | G | | | | DH | |
| CD3_SP11AVH3_VLK_3_ | GFTFSKN | 142 | IYYDSSKM | 143 | ASSVWVDLDF | 546 |
| S_PTM | G | | | | DH | |
| CD3_SP11AVH3_VLK_3_ | GFTFSKN | 142 | IYYDSSKM | 143 | ASYSVVDLDF | 549 |
| Y_SW | G | | | | DH | |
| CD3_SP11AVH3_VLK_3_ | GFTFSKN | 142 | IYYDSSKM | 143 | ASSSVVDLDF | 542 |
| S_SW | G | | | | DH | |
| CD3_SP11AVH3_VLK_3_ | GFTFSKN | 142 | IYYDSSKM | 143 | ASYSVVDLDF | 549 |
| Y_PTM_SW | G | | | | DH | |
| CD3_SP11AVH3_VLK_3_ | GFTFSKN | 142 | IYYDSSKM | 143 | ASSSVVDLDF | 542 |
| S_SWPTM | G | | | | DH | |
| CD3 SP11AVH3_VLK_SW | GFTFSKN | 142 | IYYDSSKM | 143 | ASFSVVDLDF | 465 |
| PTM | G | | | | DH | |
| CD3_SP11AVH3_VLK_3_ | GFTFSKN | 142 | IYYDSSKM | 143 | ASFSVVDLDF | 465 |
| SW | G | | | | DH | |
| CD3_sp11a_VH1_VK2_Y | GFTFSKQ | 480 | IYYDSSKM | 143 | ASYVWVDLDF | 553 |
| | G | | | | DH | |
| CD3_sp11a_VH1_VK2_S | GFTFSKQ | 480 | IYYDSSKM | 143 | ASSVWVDLDF | 546 |
| | G | | | | DH | |
| CD3_sp11a_VH1_VK2_Y_ | GFTFSKN | 142 | IYYDSSKM | 143 | ASYVWVDLDF | 553 |
| PTM | G | | | | DH | |
| CD3_sp11a_VH1_VK2_S_ | GFTFSKN | 142 | IYYDSSKM | 143 | ASSVWVDLDF | 546 |
| PTM | G | | | | DH | |
| CD3_sp11a_VH1_VK2_Y_ | GFTFSKQ | 480 | IYYDSSKM | 143 | ASYSVVDLDF | 549 |
| SW | G | | | | DH | |
| CD3_sp11a_VH1_VK2_S_ | GFTFSKQ | 480 | IYYDSSKM | 143 | ASSSVVDLDF | 542 |
| SW | G | | | | DH | |
| CD3_sp11a_VH1_VK2_Y_ | GFTFSKN | 142 | IYYDSSKM | 143 | ASYSVVDLDF | 549 |
| PTM | G | | | | DH | |
| CD3_sp11a_VH1_VK2_S_ | GFTFSKN | 142 | IYYDSSKM | 143 | ASSSVVDLDF | 542 |
| PTM_SW | G | | | | DH | |
| CD3_sp11a_VH1_VK2_S | GFTFSKQ | 480 | IYYDSSKM | 143 | ASFSVVDLDF | 465 |
| W | G | | | | DH | |
| CD3_sp11a_VH1_VK2_S | GFTFSKN | 142 | IYYDSSKM | 143 | ASFSVVDLDF | 465 |
| W_PTM | G | | | | DH | |
| CD3_SP11A_VH3_VLK1_ | GFTFSKN | 142 | IYYDSSKM | 143 | ASYVWVDLDF | 553 |
| Y | G | | | | DH | |
| CD3_SP11A_VH3_VLK1_S | GFTFSKN | 142 | IYYDSSKM | 143 | ASSVWVDLDF | 546 |
| | G | | | | DH | |
| CD3_SP11A_VH3_VLK1_ | GFTFSKQ | 480 | IYYDSSKM | 143 | ASYVWVDLDF | 553 |
| Y_PTM | G | | | | DH | |
| CD3_SP11A_VH3_VLK1_ | GFTFSKQ | 480 | IYYDSSKM | 143 | ASSVWVDLDF | 546 |
| S_PTM | G | | | | DH | |
| CD3_SP11A_VH3_VLK1_ | GFTFSKN | 142 | IYYDSSKM | 143 | ASYSVVDLDF | 549 |
| Y_SW | G | | | | DH | |
| CD3_SP11A_VH3_VLK1_ | GFTFSKN | 142 | IYYDSSKM | 143 | ASSSVVDLDF | 542 |
| S_SW | G | | | | DH | |
| CD3_SP11A_VH3_VLK1_ | GFTFSKQ | 480 | IYYDSSKM | 143 | ASYVWVDLDF | 553 |
| Y_PTM | G | | | | DH | |
| CD3_SP11A_VH3_VLK1_ | GFTFSKQ | 480 | IYYDSSKM | 143 | ASSSVVDLDF | 542 |
| S_PTM_SW | G | | | | DH | |
| CSP_11A_VH3_VLK1P_ | GFTFSKQ | 480 | IYYDSSKM | 143 | ASFSVVDLDF | 465 |
| SW | G | | | | DH | |
| CD3_SP11A_VH3_VLK1_ | GFTFSKN | 142 | IYYDSSKM | 143 | ASFSVVDLDF | 465 |
| SW | G | | | | DH | |
| CD3_SP11A_VH5_VK2_Y | GFTFSKQ | 480 | IYYDSSKM | 143 | ASYVWVDLDF | 553 |
| | G | | | | DH | |
| CD3_SP11A_VH5_VK2_S | GFTFSKQ | 480 | IYYDSSKM | 143 | ASSVWVDLDF | 546 |
| | G | | | | DH | |
| CD3_SP11A_VH5_VK2_Y_ | GFTFSKN | 142 | IYYDSSKM | 143 | ASYVWVDLDF | 553 |
| PTM | G | | | | DH | |
| CD3_SP11A_VH5_VK2_S_ | GFTFSKN | 142 | IYYDSSKM | 143 | ASSVWVDLDF | 546 |
| PTM | G | | | | DH | |
| CD3_SP11A_VH5_VK2_Y_ | GFTFSKQ | 480 | IYYDSSKM | 143 | ASYSVVDLDF | 549 |
| SW | G | | | | DH | |
| CD3_SP11A_VH5_VK2_S_ | GFTFSKQ | 480 | IYYDSSKM | 143 | ASSSVVDLDF | 542 |
| SW | G | | | | DH | |
| CD3_SP11A_VH5_VK2_Y_ | GFTFSKN | 142 | IYYDSSKM | 143 | ASYSVVDLDF | 549 |
| PTM_SW | G | | | | DH | |
| CD3_SP11A_VH5_VK2_S_ | GFTFSKN | 142 | IYYDSSKM | 143 | ASSSVVDLDF | 542 |
| PTM_SW | G | | | | DH | |
| CD3_SP11A_VH5_VK2_P | GFTFSKN | 142 | IYYDSSKM | 143 | ASFSVVDLDF | 465 |
| TM_SW | G | | | | DH | |
| CD3_SP11A_VH5_VK2_S | GFTFSKQ | 480 | IYYDSSKM | 143 | ASFSVVDLDF | 465 |
| W | G | | | | DH | |

TABLE 1F-2
CD3 Binders - Light Chain CDR sequences according to IMGT numbering scheme

| | | SEQ ID | | SEQ ID | | SEQ ID |
|---|---|---|---|---|---|---|
| Binder | CDR-L1 | NO: | CDR-L2 | NO: | CDR-L3 | NO: |
| NOV292 | QSLVRSD | 158 | RVS | 159 | LQSSHF | 160 |
| | GTTY | | | | PWT | |
| NOV123 | QSLIYSIGN | 190 | RVS | 191 | FQSTHL | 192 |
| | TY | | | | PYT | |
| Sp10b | QSLIYSIGN | 190 | RVS | 156 | FQSTHL | 183 |
| | TY | | | | PYT | |
| NOV453 | QNINNY | 222 | NTDHLQA | 223 | LQHRSR | 224 |
| | | | GVP | | YT | |
| NOV229 | QNINNY | 254 | NTDHLQA | 255 | LQHRSR | 256 |
| | | | GVP | | YT | |
| NOV110 | QSLVYSHG | 286 | RVS | 287 | FQSTHL | 288 |
| | NTY | | | | PYT | |
| NOV832 | QSLVYSHG | 318 | RVS | 319 | FQSTHL | 320 |
| | NTY | | | | PYT | |
| NOV589 | QSLVRSD | 350 | RVS | 351 | LQSSHF | 352 |
| | GTTY | | | | PWT | |
| NOV580 | QNIDKY | 382 | NTNNLEA | 383 | LQHRSS | 384 |
| | | | GVP | | YT | |
| NOV567 | QSIGNS | 414 | STSTLEY | 415 | LQYATY | 416 |
| | | | GVP | | PYT | |
| NOV221 | QNIDKY | 446 | NTNNLEA | 447 | LQHRSG | 448 |
| | | | GVP | | YT | |
| CD3_sp11a_bkm1 | QSLVRSD | 158 | RVS | 156 | LQSSHF | 151 |
| | GTTY | | | | PWT | |
| CD3_SP11a_bkm2 | QSLVRSD | 158 | RVS | 156 | LQSSHF | 151 |
| | GTTY | | | | PWT | |
| CD3_sp11a_hz0 | QSLVRSD | 158 | RVS | 156 | LQSSHF | 151 |
| | GTTY | | | | PWT | |
| CD3_SP11A_HZ1 | QSLVRSD | 158 | RVS | 156 | LQSSH | 484 |
| | GTTY | | | | | |
| CD3_sp11a_sansPTM_hz1 | QSLVRSEG | 486 | RVS | 156 | LQSSHF | 151 |
| | TTY | | | | PWT | |
| CD3_sp11a_sansPTM_rat | QSLVRSEG | 486 | RVS | 156 | LQSSHF | 151 |
| | TTY | | | | PWT | |
| CD3_sp11a_VHVL_YY | QSLVRSD | 158 | RVS | 156 | LQSSHF | 151 |
| | GTTY | | | | PWT | |
| CD3_SP11A_VHVL_SS | QSLVRSD | 158 | RVS | 156 | LQSSHF | 151 |
| | GTTY | | | | PWT | |
| CD3_SP11A_VHVL_WS | QSLVRSD | 158 | RVS | 156 | LQSSHF | 151 |
| | GTTY | | | | PWT | |
| CD3_sp11a_VHVL_SW | QSLVRSD | 158 | RVS | 156 | LQSSHF | 151 |
| | GTTY | | | | PWT | |
| CD3_SP11A_VHVL_TT | QSLVRSD | 158 | RVS | 156 | LQSSHF | 151 |
| | GTTY | | | | PWT | |
| CD3_SP11A_VHVL_TW | QSLVRSD | 158 | RVS | 156 | LQSSHF | 151 |
| | GTTY | | | | PWT | |
| CD3_SP11A_VHVL_VVT | QSLVRSD | 158 | RVS | 156 | LQSSHF | 151 |
| | GTTY | | | | PWT | |
| CD3_SP11A VH3_VLK_3 | QSLVRSEG | 486 | RVS | 156 | LQSSHF | 151 |
| | TTY | | | | PWT | |
| CD3_sp11a_VH1_VK2 | QSLVRSD | 158 | RVS | 156 | LQSSHF | 151 |
| | GTTY | | | | PWT | |
| CD3_SP11A_VH3_VLK1 | QSLVRSEG | 486 | RVS | 156 | LQSSHF | 151 |
| | TTY | | | | PWT | |
| CD3_SP11A_VH5_VK2 | QSLVRSD | 158 | RVS | 156 | LQSSHF | 151 |
| | GTTY | | | | PWT | |
| CD3_sp9aFW1_VL_VH_S5 | QNINNY | 222 | NTDHLQA | 223 | LQHRSR | 215 |
| 6G | | | GVP | | YT | |
| CD3_SP9AFW4_VL_VH_S | QNINNY | 222 | NTDHLQA | 223 | LQHRSR | 215 |
| 56G | | | GVP | | YT | |
| CD3_sp9aFW1_VLVH | QNINNY | 222 | NTDHLQA | 223 | LQHRSR | 215 |
| | | | GVP | | YT | |
| CD3_sp9aFW4_VLVH | QNINNY | 222 | NTDHLQA | 223 | LQHRSR | 215 |
| | | | GVP | | YT | |
| CD3_sp9arabtor_VHVL | QNINNY | 222 | NTDHLQA | 223 | LQHRSR | 215 |
| | | | GVP | | YT | |
| CD3_sp9arabtor_VLVH | QNINNY | 222 | NTDHLQA | 223 | LQHRSR | 215 |
| | | | GVP | | YT | |
| CD3_sp11a_VHVL_YY_SA | QSLVRSEG | 486 | RVS | 156 | LQSSHF | 151 |
| NSPTM | TTY | | | | PWT | |
| CD3_sp11a_VHVL_YY_SA | QSLVRSEG | 486 | RVS | 156 | LQSSHF | 151 |
| NSPTM_Y | TTY | | | | PWT | |
| CD3_sp11a_VHVL_YY_SA | QSLVRSEG | 486 | RVS | 156 | LQSSHF | 151 |
| NSPTM_S | TTY | | | | PWT | |
| CD3_sp11a_VHVL_YY_Y | QSLVRSD | 158 | RVS | 156 | LQSSHF | 151 |
| | GTTY | | | | PWT | |
| CD3_sp11a_VHVL_YY_s | QSLVRSD | 158 | RVS | 156 | LQSSHF | 151 |
| | GTTY | | | | PWT | |
| CD3_sp11a_VHVL_SS_SA | QSLVRSEG | 486 | RVS | 156 | LQSSHF | 151 |
| NSPTM | TTY | | | | PWT | |
| CD3_sp11a_VHVL_SS_SA | QSLVRSEG | 486 | RVS | 156 | LQSSHF | 151 |
| NSPTM_Y | TTY | | | | PWT | |
| CD3_sp11a_VHVL_SS_SA | QSLVRSEG | 486 | RVS | 156 | LQSSHF | 151 |
| NSPTM_S | TTY | | | | PWT | |
| CD3_sp11a_VHVL_SS_Y | QSLVRSD | 158 | RVS | 156 | LQSSHF | 151 |
| | GTTY | | | | PWT | |
| CD3_sp11a_VHVL_SS_S | QSLVRSD | 158 | RVS | 156 | LQSSHF | 151 |
| | GTTY | | | | PWT | |
| CD3_sp11a_VHVL_SS_ | QSLVRSEG | 486 | RVS | 156 | LQSSHF | 151 |
| SANSPTM | TTY | | | | PWT | |
| CD3_sp11a_VHVL_WS_ | QSLVRSEG | 486 | RVS | 156 | LQSSHF | 151 |
| SANSPTM_Y | TTY | | | | PWT | |
| CD3_sp11a_VHVL_WS_ | QSLVRSEG | 486 | RVS | 156 | LQSSHF | 151 |
| SANSPTM_S | TTY | | | | PWT | |
| CD3_sp11a_VHVL_WS_Y | QSLVRSD | 158 | RVS | 156 | LQSSHF | 151 |
| | GTTY | | | | PWT | |
| CD3_sp11a_VHVL_WS_S | QSLVRSD | 158 | RVS | 156 | LQSSHF | 151 |
| | GTTY | | | | PWT | |
| CD3_sp11a_VHVL_WS_ | QSLVRSEG | 486 | RVS | 156 | LQSSHF | 151 |
| SANSPTM | TTY | | | | PWT | |
| CD3_sp11a_VHVL_SW_ | QSLVRSEG | 486 | RVS | 156 | LQSSHF | 151 |
| SANSPTM_Y | TTY | | | | PWT | |
| CD3_sp11a_VHVL_SW_ | QSLVRSEG | 486 | RVS | 156 | LQSSHF | 151 |
| SANSPTM_S | TTY | | | | PWT | |
| CD3_sp11a_VHVL_SW_Y | QSLVRSD | 158 | RVS | 156 | LQSSHF | 151 |
| | GTTY | | | | PWT | |
| CD3_sp11a_VHVL_SW_S | QSLVRSD | 158 | RVS | 156 | LQSSHF | 151 |
| | GTTY | | | | PWT | |
| CD3_sp11a_VHVL_SW_ | QSLVRSEG | 486 | RVS | 156 | LQSSHF | 151 |
| SANSPTM | TTY | | | | PWT | |
| CD3_sp11a_VHVL_TW_ | QSLVRSEG | 486 | RVS | 156 | LQSSHF | 151 |
| _SANSPTM_Y | TTY | | | | PWT | |
| CD3_sp11a_VHVL_TW_ | QSLVRSEG | 486 | RVS | 156 | LQSSHF | 151 |
| SANSPTM_S | TTY | | | | PWT | |
| CD3_sp11a_VHVL_TW_Y | QSLVRSD | 158 | RVS | 156 | LQSSHF | 151 |
| | GTTY | | | | PWT | |
| CD3_sp11a_VHVL_TW_S | QSLVRSD | 158 | RVS | 156 | LQSSHF | 151 |
| | GTTY | | | | PWT | |
| CD3_sp11a_VHVL_TW_ | QSLVRSEG | 486 | RVS | 156 | LQSSHF | 151 |
| SANSPTM | TTY | | | | PWT | |
| CD3_sp11a_VHVL_TT_ | QSLVRSEG | 486 | RVS | 156 | LQSSHF | 151 |
| SANSPTM_Y | TTY | | | | PWT | |
| CD3_sp11a_VHVL_TT_SA | QSLVRSEG | 486 | RVS | 156 | LQSSHF | 151 |
| NSPTM_S | TTY | | | | PWT | |
| CD3_sp11a_VHVL_TT_Y | QSLVRSD | 158 | RVS | 156 | LQSSHF | 151 |
| | GTTY | | | | PWT | |
| CD3_sp11a_VHVL_TT_S | QSLVRSD | 158 | RVS | 156 | LQSSHF | 151 |
| | GTTY | | | | PWT | |
| CD3_sp11a_VHVL_TT_SA | QSLVRSEG | 486 | RVS | 156 | LQSSHF | 151 |
| NSPTM | TTY | | | | PWT | |
| CD3_SP11AVH3_VLK_3_Y | QSLVRSEG | 486 | RVS | 156 | LQSSHF | 151 |
| | TTY | | | | PWT | |
| CD3_SP11AVH3_VLK_3_S | QSLVRSEG | 486 | RVS | 156 | LQSSHF | 151 |
| | TTY | | | | PWT | |
| CD3_SP11AVH3_VLK_3_Y_ | QSLVRSD | 158 | RVS | 156 | LQSSHF | 151 |
| PTM | GTTY | | | | PWT | |
| CD3_SP11AVH3_VLK_3_S_ | QSLVRSD | 158 | RVS | 156 | LQSSHF | 151 |
| PTM | GTTY | | | | PWT | |
| CD3_SP11AVH3_VLK_3_Y_ | QSLVRSEG | 486 | RVS | 156 | LQSSHF | 151 |
| SW | TTY | | | | PWT | |
| CD3_SP11AVH3_VLK_3_S_ | QSLVRSEG | 486 | RVS | 156 | LQSSHF | 151 |
| SW | TTY | | | | PWT | |
| CD3_SP11AVH3_VLK_3_Y_ | QSLVRSD | 158 | RVS | 156 | LQSSHF | 151 |
| PTM_SW | GTTY | | | | PWT | |
| CD3_SP11AVH3_VLK_3_S_ | QSLVRSD | 158 | RVS | 156 | LQSSHF | 151 |
| SVVPTM | GTTY | | | | PWT | |
| CD3_SP11AVH3_VLK_SW | QSLVRSD | 158 | RVS | 156 | LQSSHF | 151 |
| PTM | GTTY | | | | PWT | |
| CD3_SP11AVH3_VLK_3_SW | QSLVRSEG | 486 | RVS | 156 | LQSSHF | 151 |
| | TTY | | | | PWT | |
| CD3_sp11a_VH1_VK2_Y | QSLVRSD | 158 | RVS | 156 | LQSSHF | 151 |
| | GTTY | | | | PWT | |
| CD3_sp11a_VH1_VK2_S | QSLVRSD | 158 | RVS | 156 | LQSSHF | 151 |
| | GTTY | | | | PWT | |
| CD3_sp11a_VH1_VK2_Y_P | QSLVRSEG | 486 | RVS | 156 | LQSSHF | 151 |
| TM | TTY | | | | PWT | |
| CD3_sp11a_VH1_VK2_S_P | QSLVRSEG | 486 | RVS | 156 | LQSSHF | 151 |
| TM | TTY | PWT | | | | |
| CD3_sp11a_VH1_VK2_Y_SW | QSLVRSD | 158 | RVS | 156 | LQSSHF | 151 |
| | GTTY | | | | PWT | |
| CD3_sp11a_VH1_VK2_S_SW | QSLVRSD | 158 | RVS | 156 | LQSSHF | 151 |
| | GTTY | | | | PWT | |
| CD3_sp11a_VH1_VK2_Y_P | QSLVRSEG | 486 | RVS | 156 | LQSSHF | 151 |
| TM | TTY | | | | PWT | |
| CD3_sp11a_VH1_VK2_S_P | QSLVRSEG | 486 | RVS | 156 | LQSSHF | 151 |
| TM_SW | TTY | | | | PWT | |
| CD3_sp11a_VH1_VK2_SW | QSLVRSD | 158 | RVS | 156 | LQSSHF | 151 |
| | GTTY | | | | PWT | |
| CD3_sp11a_VH1_VK2_SW_ | QSLVRSDE | 568 | RVS | 156 | LQSSHF | 151 |
| PTM | TTY | | | | PWT | |
| CD3_SP11A_VH3_VLK1_Y | QSLVRSEG | 486 | RVS | 156 | LQSSHF | 151 |
| | TTY | | | | PWT | |
| CD3_SP11A_VH3_VLK1_S | QSLVRSEG | 486 | RVS | 156 | LQSSHF | 151 |
| | TTY | | | | PWT | |
| CD3_SP11A_VH3_VLK1_Y_ | QSLVRSD | 158 | RVS | 156 | LQSSHF | 151 |
| PTM | GTTY | | | | PWT | |
| CD3_SP11A_VH3_VLK1_S_ | QSLVRSD | 158 | RVS | 156 | LQSSHF | 151 |
| PTM | GTTY | | | | PWT | |
| CD3_SP11A_VH3_VLK1_Y_ | QSLVRSEG | 486 | RVS | 156 | LQSSHF | 151 |
| SW | TTY | | | | PWT | |
| CD3_SP11A_VH3_VLK1_S_ | QSLVRSEG | 486 | RVS | 156 | LQSSHF | 151 |
| SW | TTY | | | | PWT | |
| CD3_SP11A_VH3_VLK1_Y_ | QSLVRSD | 158 | RVS | 156 | LQSSHF | 151 |
| PTM | GTTY | | | | PWT | |
| CD3_SP11A_VH3_VLK1_S_ | QSLVRSD | 158 | RVS | 156 | LQSSHF | 151 |
| PTM_SW | GTTY | | | | PWT | |
| CD3_SP11A_VH3_VLK1PT | QSLVRSD | 158 | RVS | 156 | LQSSHF | 151 |
| M_SW | GTTY | | | | PWT | |
| CD3_SP11A_VH3_VLK1_SW | QSLVRSEG | 486 | RVS | 156 | LQSSHF | 151 |
| | TTY | | | | PWT | |
| CD3_SP11A_VH5_VK2_Y | QSLVRSD | 158 | RVS | 156 | LQSSHF | 151 |
| | GTTY | | | | PWT | |
| CD3_SP11A_VH5_VK2_S | QSLVRSD | 158 | RVS | 156 | LQSSHF | 151 |
| | GTTY | | | | PWT | |
| CD3_SP11A_VH5_VK2_Y_ | QSLVRSD | 158 | RVS | 156 | LQSSHF | 151 |
| PTM | GTTY | | | | PWT | |
| CD3_SP11A_VH5_VK2_S_ | QSLVRSD | 158 | RVS | 156 | LQSSHF | 151 |
| PTM | GTTY | | | | PWT | |
| CD3_SP11A_VH5_VK2_Y_ | QSLVRSD | 158 | RVS | 156 | LQSSHF | 151 |
| SW | GTTY | | | | PWT | |
| CD3_SP11A_VH5_VK2_S_ | QSLVRSD | 158 | RVS | 156 | LQSSHF | 151 |
| SW | GTTY | | | | PWT | |
| CD3_SP11A_VH5_VK2_Y_ | QSLVRSD | 158 | RVS | 156 | LQSSHF | 151 |
| PTM_SW | GTTY | | | | PWT | |
| CD3_SP11A_VH5_VK2_S_ | QSLVRSD | 158 | RVS | 156 | LQSSHF | 151 |
| PTM_SW | GTTY | | | | PWT | |
| CD3_SP11A_VH5_VK2_PT | QSLVRSD | 158 | RVS | 156 | LQSSHF | 151 |
| M_SW | GTTY | | | | PWT | |
| CD3_SP11A_VH5_VK2_SW | QSLVRSD | 158 | RVS | 156 | LQSSHF | 151 |
| | GTTY | | | | PWT | |

TABLE 1G-1
CD3 Binders-Heavy Chain CDR sequences according to combination of Kabat and
Chothia numbering schemes

| | | SEQ ID | | SEQ ID | | SEQ ID |
|---|---|---|---|---|---|---|
| Binder | CDR-H1 | NO: | CDR-H2 | NO: | CDR-H3 | NO: |
| NOV292 | GFTFSK | 133 | MIYYDSSKMY | 134 | FVWVDLDFD | 135 |
| | NGMH | | YADTVKG | | H | |
| NOV123 | GYTFTS | 165 | YIYPGHDAIYY | 166 | PNTMMAPLA | 167 |
| | YYIY | | SENFKG | | Y | |
| Sp10b | GYTFTS | 165 | YIYPGHDAIYY | 166 | PNTMMAPLA | 167 |
| | YYIY | | SENFKG | | Y | |
| NOV453 | GFSLTT | 197 | RMRYSGDTSF | 198 | DPMYIPNYS | 199 |
| | YNVH | | NAALTS | | YGVMNA | |
| NOV229 | GFSLTT | 229 | RMRYSGDTSF | 230 | DPMYIPNYS | 231 |
| | YNVH | | NAALTS | | YGVMNA | |
| NOV110 | GYTFTS | 261 | YIYPANGGIYY | 262 | PVTMMAPLV | 263 |
| | YYIY | | SEKFKG | | F | |
| NOV832 | GYTFTS | 293 | YIYPANGGIYY | 294 | PVTMMAPLV | 295 |
| | YYIY | | SEKFKG | | F | |
| NOV589 | GFTFSK | 325 | MIYYDSSRMY | 326 | FVWVDLDFDY | 327 |
| | NGMH | | YADTVKG | | | |
| NOV580 | GFSLTT | 357 | RMRYSGDTSY | 358 | DPMYIPGYS | 359 |
| | YNIH | | SSALKS | | YGVMNA | |
| NOV567 | GFAFRK | 389 | LIYYDSSKMNY | 390 | LNSEYD | 391 |
| | YGMS | | ADTVKG | | | |
| NOV221 | GFSLTT | 421 | RMRYSGDTSY | 422 | DPMYIPGYS | 423 |
| | YNIH | | SSALKS | | YGVMNA | |
| CD3_sp11a_bkm1 | GFTFSK | 133 | MIYYDSSKMY | 134 | FVWVDLDFD | 135 |
| | NGMH | | YADTVKG | | H | |
| CD3_SP11a_bkm2 | GFTFSK | 133 | MIYYDSSKMY | 134 | FVWVDLDFD | 135 |
| | NGMH | | YADTVKG | | H | |
| CD3_sp11a_hz0 | GFTFSK | 133 | MIYYDSSKMY | 134 | FVWVDLDFD | 135 |
| | NGMH | | YADTVKG | | H | |
| CD3_SP11A_HZ1 | GFTFSK | 133 | MIYYDSSKMY | 134 | FVWVDLDFD | 135 |
| | NGMH | | YADTVKG | | H | |
| CD3_sp11a_sansPTM_h | GFTFSK | 481 | MIYYDSSKMY | 134 | FVWVDLDFD | 135 |
| z1 | QGMH | | YADTVKG | | H | |
| CD3 sp11a_sansPTM_r | GFTFSK | 481 | MIYYDSSKMY | 134 | FVWVDLDFD | 135 |
| at | QGMH | | YADTVKG | | H | |
| CD3_sp11a_VHVL_YY | GFTFSK | 133 | MIYYDSSKMY | 134 | FYYDLDFDH | 478 |
| | NGMH | | YADTVKG | | | |
| CD3_SP11A_VHVL_SS | GFTFSK | 133 | MIYYDSSKMY | 134 | FSSDLDFDH | 472 |
| | NGMH | | YADTVKG | | | |
| CD3_SP11A_VHVL_WS | GFTFSK | 133 | MIYYDSSKMY | 134 | FWSDLDFDH | 476 |
| | NGMH | | YADTVKG | | | |
| CD3_sp11a_VHVL_SW | GFTFSK | 133 | MIYYDSSKMY | 134 | FSVVDLDFDH | 473 |
| | NGMH | | YADTVKG | | | |
| CD3_SP11A_VHVL_TT | GFTFSK | 133 | MIYYDSSKMY | 134 | FTTDLDFDH | 474 |
| | NGMH | | YADTVKG | | | |
| CD3_SP11A_VHVL_TW | GFTFSK | 133 | MIYYDSSKMY | 134 | FTWDLDFDH | 475 |
| | NGMH | | YADTVKG | | | |
| CD3_SP11A_VHVL_VVT | GFTFSK | 133 | MIYYDSSKMY | 134 | FVVTDLDFDH | 477 |
| | NGMH | | YADTVKG | | | |
| CD3_SP11A | GFTFSK | 133 | MIYYDSSKMY | 134 | FVWVDLDFD | 135 |
| VH3_VLK_3 | NGMH | | YADTVKG | | H | |
| CD3_sp11a_VH1_VK2 | GFTFSK | 481 | MIYYDSSKMY | 134 | FVWVDLDFD | 135 |
| | QGMH | | YADTVKG | | H | |
| CD3_SP11A_VH3_VLK1 | GFTFSK | 133 | MIYYDSSKMY | 134 | FVWVDLDFD | 135 |
| | NGMH | | YADTVKG | | H | |
| CD3_SP11A_VH5_VK2 | GFTFSK | 481 | MIYYDSSKMY | 134 | FVWVDLDFD | 135 |
| | QGMH | | YADTVKG | | H | |
| CD3_sp9aFW1_VL_VH_ | GFSLTT | 197 | RMRYSGDTSF | 198 | DPMYIPNYA | 471 |
| S56G | YNVH | | NAALTS | | YGVMNA | |
| CD3_SP9AFW4_VL_VH_ | GFSLTT | 197 | RMRYSGDTSF | 198 | DPMYIPNYA | 471 |
| S56G | YNVH | | NAALTS | | YGVMNA | |
| CD3_sp9aFW1_VLVH | GFSLTT | 197 | RMRYSGDTSF | 198 | DPMYIPNYA | 471 |
| | YNVH | | NAALTS | | YGVMNA | |
| CD3_sp9aFW4_VLVH | GFSLTT | 197 | RMRYSGDTSF | 198 | DPMYIPNYA | 471 |
| | YNVH | | NAALTS | | YGVMNA | |
| CD3_sp9arabtor_VHVL | GFSLTT | 197 | RMRYSGDTSF | 198 | DPMYIPNYA | 471 |
| | YNVH | | NAALTS | | YGVMNA | |
| CD3_sp9arabtor VLVH | GFSLTT | 197 | RMRYSGDTSF | 198 | DPMYIPNYA | 471 |
| | YNVH | | NAALTS | | YGVMNA | |
| CD3_sp11a_VHVL_YY_ | GFTFSK | 481 | MIYYDSSKMY | 134 | FVWVDLDFD | 135 |
| SAN_SPTM | QGMH | | YADTVKG | | H | |
| CD3_sp11a_VHVL_YY_ | GFTFSK | 481 | MIYYDSSKMY | 134 | FVWVDLDFD | 135 |
| SANSPTM_Y | QGMH | | YADTVKG | | H | |
| CD3_sp11a_VHVL_YY_ | GFTFSK | 481 | MIYYDSSKMY | 134 | FVWVDLDFD | 135 |
| SANSPTM_S | QGMH | | YADTVKG | | H | |
| CD3_sp11a_VHVL_YY_Y | GFTFSK | 133 | MIYYDSSKMY | 134 | FVWVDLDFD | 135 |
| | NGMH | | YADTVKG | | H | |
| CD3_sp11a_VHVL_YY_S | GFTFSK | 133 | MIYYDSSKMY | 134 | FVWVDLDFD | 135 |
| | NGMH | | YADTVKG | | H | |
| CD3_sp11a_VHVL_SS_ | GFTFSK | 481 | MIYYDSSKMY | 134 | FVWVDLDFD | 135 |
| SANSPTM | QGMH | | YADTVKG | | H | |
| CD3_sp11a_VHVL_SS_ | GFTFSK | 481 | MIYYDSSKMY | 134 | FVWVDLDFD | 135 |
| SANSPTM_Y | QGMH | | YADTVKG | | H | |
| CD3_sp11a_VHVL_SS_ | GFTFSK | 481 | MIYYDSSKMY | 134 | FVWVDLDFD | 135 |
| SANSPTM_S | QGMH | | YADTVKG | | H | |
| CD3_sp11a_VHVL_SS_Y | GFTFSK | 133 | MIYYDSSKMY | 134 | FVWVDLDFD | 135 |
| | NGMH | | YADTVKG | | H | |
| CD3_sp11a_VHVL_SS_S | GFTFSK | 133 | MIYYDSSKMY | 134 | FVWVDLDFD | 135 |
| | NGMH | | YADTVKG | | H | |
| CD3_sp11a_VHVL_SS_ | GFTFSK | 481 | MIYYDSSKMY | 134 | FVWVDLDFD | 135 |
| SANSPTM | QGMH | | YADTVKG | | H | |
| CD3_sp11a_VHVL_WS_ | GFTFSK | 481 | MIYYDSSKMY | 134 | FVWVDLDFD | 135 |
| SANSPTM_Y | QGMH | | YADTVKG | | H | |
| CD3_sp11a_VHVL_WS_ | GFTFSK | 481 | MIYYDSSKMY | 134 | FVWVDLDFD | 135 |
| SANSPTM_S | QGMH | | YADTVKG | | H | |
| CD3_sp11a_VHVL_WS_ | GFTFSK | 133 | MIYYDSSKMY | 134 | FVWVDLDFD | 135 |
| Y | NGMH | | YADTVKG | | H | |
| CD3_sp11a_VHVL_WS_ | GFTFSK | 133 | MIYYDSSKMY | 134 | FVWVDLDFD | 135 |
| S | NGMH | | YADTVKG | | H | |
| CD3_sp11a_VHVL_WS_ | GFTFSK | 481 | MIYYDSSKMY | 134 | FVWVDLDFD | 135 |
| SANSPTM | QGMH | | YADTVKG | | H | |
| CD3_sp11a_VHVL_SW_ | GFTFSK | 481 | MIYYDSSKMY | 134 | FVWVDLDFD | 135 |
| SANSPTM _Y | QGMH | | YADTVKG | | H | |
| CD3_sp11a_VHVL_SW_ | GFTFSK | 481 | MIYYDSSKMY | 134 | FVWVDLDFD | 135 |
| SANSPTM _S | QGMH | | YADTVKG | | H | |
| CD3_sp11a_VHVL_SW_ | GFTFSK | 133 | MIYYDSSKMY | 134 | FVWVDLDFD | 135 |
| Y | NGMH | | YADTVKG | | H | |
| CD3_sp11a_VHVL_SW_ | GFTFSK | 133 | MIYYDSSKMY | 134 | FVWVDLDFD | 135 |
| S | NGMH | | YADTVKG | | H | |
| CD3_sp11a_VHVL_SW_ | GFTFSK | 481 | MIYYDSSKMY | 134 | FVWVDLDFD | 135 |
| SANSPTM | QGMH | | YADTVKG | | H | |
| CD3_sp11a_VHVL_TW_ | GFTFSK | 481 | MIYYDSSKMY | 134 | FVWVDLDFD | 135 |
| SANSPTM _Y | QGMH | | YADTVKG | | H | |
| CD3_sp11a_VHVL_TW_ | GFTFSK | 481 | MIYYDSSKMY | 134 | FVWVDLDFD | 135 |
| SANSPTM _S | QGMH | | YADTVKG | | H | |
| CD3_sp11a_VHVL_TW_ | GFTFSK | 133 | MIYYDSSKMY | 134 | FVWVDLDFD | 135 |
| Y | NGMH | | YADTVKG | | H | |
| CD3_sp11a_VHVL_TW_ | GFTFSK | 133 | MIYYDSSKMY | 134 | FVWVDLDFD | 135 |
| S | NGMH | | YADTVKG | | H | |
| CD3_sp11a_VHVL_TW_ | GFTFSK | 481 | MIYYDSSKMY | 134 | FVWVDLDFD | 135 |
| SANSPTM | QGMH | | YADTVKG | | H | |
| CD3_sp11a_VHVL_TT_ | GFTFSK | 481 | MIYYDSSKMY | 134 | FVWVDLDFD | 135 |
| SANSPTM _Y | QGMH | | YADTVKG | | H | |
| CD3_sp11a_VHVL_TT_ | GFTFSK | 481 | MIYYDSSKMY | 134 | FVWVDLDFD | 135 |
| SANSPTM_S | QGMH | | YADTVKG | | H | |
| CD3_sp11a_VHVL_TT_ | GFTFSK | 133 | MIYYDSSKMY | 134 | FVWVDLDFD | 135 |
| Y | NGMH | | YADTVKG | | H | |
| CD3_sp11a_VHVL_TT_ | GFTFSK | 133 | MIYYDSSKMY | 134 | FVWVDLDFD | 135 |
| S | NGMH | | YADTVKG | | H | |
| CD3_sp11a_VHVL_TT_ | GFTFSK | 481 | MIYYDSSKMY | 134 | FVWVDLDFD | 567 |
| SANSPTM | QGMH | | YADTVKG | | H | |
| CD3_SP11AVH3_VLK_3_ | GFTFSK | 133 | MIYYDSSKMY | 134 | YVWVDLDFD | 626 |
| Y | NGMH | | YADTVKG | | H | |
| CD3_SP11AVH3_VLK_3_ | GFTFSK | 133 | MIYYDSSKMY | 134 | SVWVDLDFD | 618 |
| S | NGMH | | YADTVKG | | H | |
| CD3_SP11AVH3_VLK_3 | GFTFSK | 133 | MIYYDSSKMY | 134 | YVWVDLDFD | 626 |
| Y_PTM | NGMH | | YADTVKG | | H | |
| CD3_SP11AVH3_VLK_3_ | GFTFSK | 133 | MIYYDSSKMY | 134 | SVWVDLDFD | 618 |
| S_PTM | NGMH | | YADTVKG | | H | |
| CD3_SP11AVH3_VLK_3 | GFTFSK | 133 | MIYYDSSKMY | 134 | YSWDLDFDH | 621 |
| Y_SW | NGMH | | YADTVKG | | | |
| CD3_SP11AVH3_VLK_3_ | GFTFSK | 133 | MIYYDSSKMY | 134 | SSWDLDFDH | 614 |
| SW | NGMH | | YADTVKG | | | |
| CD3_SP11AVH3_VLK_3_ | GFTFSK | 133 | MIYYDSSKMY | 134 | YSWDLDFDH | 621 |
| _Y_PTM_SW | NGMH | | YADTVKG | | | |
| CD3_SP11AVH3_VLK_3_ | GFTFSK | 133 | MIYYDSSKMY | 134 | SSWDLDFDH | 614 |
| S_SVVPTM | NGMH | | YADTVKG | | | |
| CD3_SP11AVH3_VLK_S | GFTFSK | 133 | MIYYDSSKMY | 134 | FSVVDLDFDH | 473 |
| | VVPTM | | NGMH | | YADTVKG | |
| CD3_SP11AVH3_VLK_3_ | GFTFSK | 133 | MIYYDSSKMY | 134 | FSVVDLDFDH | 473 |
| SW | NGMH | | YADTVKG | | | |
| CD3_sp11a_VH1_VK2_ | GFTFSK | 481 | MIYYDSSKMY | 134 | YVWVDLDFD | 626 |
| Y | QGMH | | YADTVKG | | H | |
| CD3_sp11a_VH1_VK2_ | GFTFSK | 481 | MIYYDSSKMY | 134 | SVWVDLDFD | 618 |
| S | QGMH | | YADTVKG | | H | |
| CD3_sp11a_VH1_VK2_ | GFTFSK | 133 | MIYYDSSKMY | 134 | YVWVDLDFD | 626 |
| Y_PTM | NGMH | | YADTVKG | | H | |
| CD3_sp11a_VH1_VK2_ | GFTFSK | 133 | MIYYDSSKMY | 134 | SVWVDLDFD | 618 |
| S_PTM | NGMH | | YADTVKG | | H | |
| CD3_sp11a_VH1_VK2_ | GFTFSK | 481 | MIYYDSSKMY | 134 | YSWDLDFDH | 621 |
| Y_SW | QGMH | | YADTVKG | | | |
| CD3_sp11a_VH1_VK2_ | GFTFSK | 481 | MIYYDSSKMY | 134 | SSWDLDFDH | 614 |
| S_SW | QGMH | | YADTVKG | | | |
| CD3_sp11a_VH1_VK2_ | GFTFSK | 133 | MIYYDSSKMY | 134 | YSWDLDFDH | 621 |
| Y_PTM | NGMH | | YADTVKG | | | |
| CD3_sp11a_VH1_VK2_ | GFTFSK | 133 | MIYYDSSKMY | 134 | SSWDLDFDH | 614 |
| S_PTM_SW | NGMH | | YADTVKG | | | |
| CD3_sp11a_VH1_VK2_ | GFTFSK | 481 | MIYYDSSKMY | 134 | FSVVDLDFDH | 473 |
| SW | QGMH | | YADTVKG | | | |
| CD3_sp11a_VH1_VK2_ | GFTFSK | 133 | MIYYDSSKMY | 134 | FSVVDLDFDH | 473 |
| SW PTM | NGMH | | YADTVKG | | | |
| CD3_SP11A_VH3_VLK1_ | GFTFSK | 133 | MIYYDSSKMY | 134 | YVWVDLDFD | 626 |
| Y | NGMH | | YADTVKG | | H | |
| CD3_SP11A_VH3_VLK1_ | GFTFSK | 133 | MIYYDSSKMY | 134 | SVWVDLDFD | 618 |
| S | NGMH | | YADTVKG | | H | |
| CD3_SP11A_VH3_VLK1_ | GFTFSK | 481 | MIYYDSSKMY | 134 | YVWVDLDFD | 626 |
| Y_PTM | QGMH | | YADTVKG | | H | |
| CD3_SP11A_VH3_VLK1_ | GFTFSK | 481 | MIYYDSSKMY | 134 | SVWVDLDFD | 618 |
| S_PTM | QGMH | | YADTVKG | | H | |
| CD3_SP11A_VH3_VLK1_ | GFTFSK | 133 | MIYYDSSKMY | 134 | YSWDLDFDH | 621 |
| Y_SW | NGMH | | YADTVKG | | | |
| CD3_SP11A_VH3_VLK1_ | GFTFSK | 133 | MIYYDSSKMY | 134 | SSWDLDFDH | 614 |
| S_SW | NGMH | | YADTVKG | | | |
| CD3_SP11A_VH3_VLK1_ | GFTFSK | 481 | MIYYDSSKMY | 134 | YVWVDLDFD | 626 |
| Y_PTM | QGMH | | YADTVKG | | H | |
| CD3_SP11A_VH3_VLK1_ | GFTFSK | 481 | MIYYDSSKMY | 134 | SSWDLDFDH | 614 |
| S_PTM_SW | QGMH | | YADTVKG | | | |
| CD3_SP11A_VH3_VLK1 | GFTFSK | 481 | MIYYDSSKMY | 134 | FSVVDLDFDH | 473 |
| PTM_SW | QGMH | | YADTVKG | | | |
| CD3_SP11A_VH3_VLK1_ | GFTFSK | 133 | MIYYDSSKMY | 134 | FSVVDLDFDH | 473 |
| SW | NGMH | | YADTVKG | | | |
| CD3_SP11A_VH5_VK2_ | GFTFSK | 481 | MIYYDSSKMY | 134 | YVWVDLDFD | 626 |
| Y | QGMH | | YADTVKG | | H | |
| CD3_SP11A_VH5_VK2_ | GFTFSK | 481 | MIYYDSSKMY | 134 | SVWVDLDFD | 618 |
| S | QGMH | | YADTVKG | | H | |
| CD3_SP11A_VH5_VK2_ | GFTFSK | 133 | MIYYDSSKMY | 134 | YVWVDLDFD | 626 |
| Y_PTM | NGMH | | YADTVKG | | H | |
| CD3_SP11A_VH5_VK2_ | GFTFSK | 133 | MIYYDSSKMY | 134 | SVWVDLDFD | 618 |
| S_PTM | NGMH | | YADTVKG | | H | |
| CD3_SP11A_VH5_VK2_ | GFTFSK | 481 | MIYYDSSKMY | 134 | YSWDLDFDH | 621 |
| Y_SW | QGMH | | YADTVKG | | | |
| CD3_SP11A_VH5_VK2_ | GFTFSK | 481 | MIYYDSSKMY | 134 | SSWDLDFDH | 614 |
| S_SW | QGMH | | YADTVKG | | | |
| CD3_SP11A_VH5_VK2_ | GFTFSK | 133 | MIYYDSSKMY | 134 | YSWDLDFDH | 621 |
| Y_PTM_SW | NGMH | | YADTVKG | | | |
| CD3_SP11A_VH5_VK2_ | GFTFSK | 133 | MIYYDSSKMY | 134 | SSWDLDFDH | 614 |
| S_PTM_SW | NGMH | | YADTVKG | | | |
| CD3_SP11A_VH5_VK2_ | GFTFSK | 133 | MIYYDSSKMY | 134 | FSVVDLDFDH | 473 |
| PTM_SW | NGMH | | YADTVKG | | | |
| CD3_SP11A_VH5_VK2_ | GFTFSK | 481 | MIYYDSSKMY | 134 | FSVVDLDFDH | 473 |
| SW | QGMH | | YADTVKG | | | |

TABLE 1G-2
CD3 Binders - Light Chain CDR sequences according to combination of Kabat
and Chothia numbering schemes

| | | SEQ ID | CDR- | SEQ ID | | SEQ ID |
|---|---|---|---|---|---|---|
| Binder | CDR-L1 | NO: | L2 | NO: | CDR-L3 | NO: |
| NOV292 | RSSQSLVRSDG | 149 | RVSN | 150 | LQSSHFP | 151 |
| | TTYFN | | RFS | | WT | |
| NOV123 | RSSQSLIYSIGN | 181 | RVSN | 182 | FQSTHLP | 183 |
| | TYLH | | RFS | | YT | |
| Sp10b | RSSQSLIYSIGN | 181 | RVSN | 150 | FQSTHLP | 183 |
| | TYLH | | RFS | | YT | |
| NOV453 | KASQNINNYLN | 213 | NTDHL | 214 | LQHRSRY | 215 |
| | | | QA | | T | |
| NOV229 | KASQNINNYLN | 245 | NTDHL | 246 | LQHRSRY | 247 |
| | | | QA | | T | |
| NOV110 | RSSQSLVYSHG | 277 | RVSN | 278 | FQSTHLP | 279 |
| | NTYLH | | RFS | | YT | |
| NOV832 | RSSQSLVYSHG | 309 | RVSN | 310 | FQSTHLP | 311 |
| | NTYLH | | RFS | | YT | |
| NOV589 | RSSQSLVRSDG | 341 | RVSN | 342 | LQSSHFP | 343 |
| | TTYFN | | RFS | | WT | |
| NOV580 | KTSQNIDKYLN | 373 | NTNNL | 374 | LQHRSSY | 375 |
| | | | EA | | T | |
| NOV567 | RGSQSIGNSLN | 405 | STSTL | 406 | LQYATYP | 407 |
| | | | EY | | YT | |
| NOV221 | KSSQNIDKYLN | 437 | NTNNL | 438 | LQHRSGY | 439 |
| | | | EA | | T | |
| CD3_sp11a_bkm1 | RSSQSLVRSDG | 149 | RVSN | 150 | LQSSHFP | 151 |
| | TTYFN | | RFS | | WT | |
| CD3_SP11a_bkm2 | RSSQSLVRSDG | 149 | RVSN | 150 | LQSSHFP | 151 |
| | TTYFN | | RFS | | WT | |
| CD3_sp11a_hz0 | RSSQSLVRSDG | 149 | RVSN | 150 | LQSSHFP | 151 |
| | TTYFN | | RFS | | WT | |
| CD3_SP11A_HZ1 | RSSQSLVRSDG | 149 | RVSN | 150 | LQSSH | 484 |
| | TTYFN | | RFS | | | |
| CD3_sp11a_sansPTM_hz1 | RSSQSLVRSEG | 487 | RVSN | 150 | LQSSHFP | 151 |
| | TTYFN | | RFS | | WT | |
| CD3_sp11a_sansPTM_rat | RSSQSLVRSEG | 487 | RVSN | 150 | LQSSHFP | 151 |
| | TTYFN | | RFS | | WT | |
| CD3_sp11a_VHVL_YY | RSSQSLVRSDG | 149 | RVSN | 150 | LQSSHFP | 151 |
| | TTYFN | | RFS | | WT | |
| CD3_SP11A_VHVL_SS | RSSQSLVRSDG | 149 | RVSN | 150 | LQSSHFP | 151 |
| | TTYFN | | RFS | | WT | |
| CD3_SP11A_VHVL_WS | RSSQSLVRSDG | 149 | RVSN | 150 | LQSSHFP | 151 |
| | TTYFN | | RFS | | WT | |
| CD3_sp11a_VHVL_SW | RSSQSLVRSDG | 149 | RVSN | 150 | LQSSHFP | 151 |
| | TTYFN | | RFS | | WT | |
| CD3_SP11A_VHVL_TT | RSSQSLVRSDG | 149 | RVSN | 150 | LQSSHFP | 151 |
| | TTYFN | | RFS | | WT | |
| CD3_SP11A_VHVL_TW | RSSQSLVRSDG | 149 | RVSN | 150 | LQSSHFP | 151 |
| | TTYFN | | RFS | | WT | |
| CD3_SP11A_VHVL_VVT | RSSQSLVRSDG | 149 | RVSN | 150 | LQSSHFP | 151 |
| | TTYFN | | RFS | | WT | |
| CD3_SP11A VH3_VLK_3 | RSSQSLVRSEG | 487 | RVSN | 150 | LQSSHFP | 151 |
| | TTYFN | | RFS | | WT | |
| CD3_sp11a_VH1_VK2 | RSSQSLVRSDG | 149 | RVSN | 150 | LQSSHFP | 151 |
| | TTYFN | | RFS | | WT | |
| CD3_SP11A_VH3_VLK1 | RSSQSLVRSEG | 487 | RVSN | 150 | LQSSHFP | 151 |
| | TTYFN | | RFS | | WT | |
| CD3_SP11A_VH5_VK2 | RSSQSLVRSDG | 149 | RVSN | 150 | LQSSHFP | 151 |
| | TTYFN | | RFS | | WT | |
| CD3_sp9aFW1_VL_VH_S5 | KASQNINNYLN | 213 | NTDHL | 214 | LQHRSRY | 215 |
| 6G | | | QA | | T | |
| CD3_SP9AFW4_VL_VH_S | KASQNINNYLN | 213 | NTDHL | 214 | LQHRSRY | 215 |
| 56G | | | QA | | T | |
| CD3_sp9aFW1_VLVH | KASQNINNYLN | 213 | NTDHL | 214 | LQHRSRY | 215 |
| | | | QA | | T | |
| CD3_sp9aFW4_VLVH | KASQNINNYLN | 213 | NTDHL | 214 | LQHRSRY | 215 |
| | | | QA | | T | |
| CD3_sp9arabtor_VHVL | KASQNINNYLN | 213 | NTDHL | 214 | LQHRSRY | 215 |
| | | | QA | | T | |
| CD3_sp9arabtor_VLVH | KASQNINNYLN | 213 | NTDHL | 214 | LQHRSRY | 215 |
| | | | QA | | T | |
| CD3_sp11a_VHVL_YY_SA | RSSQSLVRSEG | 487 | RVSN | 150 | LQSSHFP | 151 |
| NSPTM | TTYFN | | RFS | | WT | |
| CD3_sp11a_VHVL_YY_SA | RSSQSLVRSEG | 487 | RVSN | 150 | LQSSHFP | 151 |
| NSPTM_Y | TTYFN | | RFS | | WT | |
| CD3_sp11a_VHVL_YY_SA | RSSQSLVRSEG | 487 | RVSN | 150 | LQSSHFP | 151 |
| NSPTM_S | TTYFN | | RFS | | WT | |
| CD3_sp11a_VHVL_YY_Y | RSSQSLVRSDG | 149 | RVSN | 150 | LQSSHFP | 151 |
| | TTYFN | | RFS | | WT | |
| CD3_sp11a_VHVL_YY_s | RSSQSLVRSDG | 149 | RVSN | 150 | LQSSHFP | 151 |
| | TTYFN | | RFS | | WT | |
| CD3_sp11a_VHVL_SS_SA | RSSQSLVRSEG | 487 | RVSN | 150 | LQSSHFP | 151 |
| NSPTM | TTYFN | | RFS | | WT | |
| CD3_sp11a_VHVL_SS_SA | RSSQSLVRSEG | 487 | RVSN | 150 | LQSSHFP | 151 |
| NSPTM_Y | TTYFN | | RFS | | WT | |
| CD3_sp11a_VHVL_SS_SA | RSSQSLVRSEG | 487 | RVSN | 150 | LQSSHFP | 151 |
| NSPTM_S | TTYFN | | RFS | | WT | |
| CD3_sp11a_VHVL_SS_Y | RSSQSLVRSDG | 149 | RVSN | 150 | LQSSHFP | 151 |
| | TTYFN | | RFS | | WT | |
| CD3_sp11a_VHVL_SS_S | RSSQSLVRSDG | 149 | RVSN | 150 | LQSSHFP | 151 |
| | TTYFN | | RFS | | WT | |
| CD3_sp11a_VHVL_ SS_ | RSSQSLVRSEG | 487 | RVSN | 150 | LQSSHFP | 151 |
| SANSPTM | TTYFN | | RFS | | WT | |
| CD3_sp11a_VHVL_WS_ | RSSQSLVRSEG | 487 | RVSN | 150 | LQSSHFP | 151 |
| SANSPTM_Y | TTYFN | | RFS | | WT | |
| CD3_sp11a_VHVL_WS_ | RSSQSLVRSEG | 487 | RVSN | 150 | LQSSHFP | 151 |
| SANSPTM _S | TTYFN | | RFS | | WT | |
| CD3_sp11a_VHVL_WS_Y | RSSQSLVRSDG | 149 | RVSN | 150 | LQSSHFP | 151 |
| | TTYFN | | RFS | | WT | |
| CD3_sp11a_VHVL_WS_S | RSSQSLVRSDG | 149 | RVSN | 150 | LQSSHFP | 151 |
| | TTYFN | | RFS | | WT | |
| CD3_sp11a_VHVL_WS_ | RSSQSLVRSEG | 487 | RVSN | 150 | LQSSHFP | 151 |
| SANSPTM | TTYFN | | RFS | | WT | |
| CD3_sp11a_VHVL_SW_ | RSSQSLVRSEG | 487 | RVSN | 150 | LQSSHFP | 151 |
| SANSPTM _Y | TTYFN | | RFS | | WT | |
| CD3_sp11a_VHVL_SW_ | RSSQSLVRSEG | 487 | RVSN | 150 | LQSSHFP | 151 |
| SANSPTM_S | TTYFN | | RFS | | WT | |
| CD3_sp11a_VHVL_SW_Y | RSSQSLVRSDG | 149 | RVSN | 150 | LQSSHFP | 151 |
| | TTYFN | | RFS | | WT | |
| CD3_sp11a_VHVL_SW_S | RSSQSLVRSDG | 149 | RVSN | 150 | LQSSHFP | 151 |
| | TTYFN | | RFS | | WT | |
| CD3_sp11a_VHVL_SW_ | RSSQSLVRSEG | 487 | RVSN | 150 | LQSSHFP | 151 |
| SANSPTM | TTYFN | | RFS | | WT | |
| CD3_sp11a_VHVL_TW_ | RSSQSLVRSEG | 487 | RVSN | 150 | LQSSHFP | 151 |
| SANSPTM_Y | TTYFN | | RFS | | WT | |
| CD3_sp11a_VHVL_TW | RSSQSLVRSEG | 487 | RVSN | 150 | LQSSHFP | 151 |
| SANSPTM_S | TTYFN | | RFS | | WT | |
| CD3_sp11a_VHVL_TW_Y | RSSQSLVRSDG | 149 | RVSN | 150 | LQSSHFP | 151 |
| | TTYFN | | RFS | | WT | |
| CD3_sp11a_VHVL_TW_S | RSSQSLVRSDG | 149 | RVSN | 150 | LQSSHFP | 151 |
| | TTYFN | | RFS | | WT | |
| CD3_sp11a_VHVL_TW_ | RSSQSLVRSEG | 487 | RVSN | 150 | LQSSHFP | 151 |
| SANSPTM | TTYFN | | RFS | | WT | |
| CD3_sp11a_VHVL_TT_ | RSSQSLVRSEG | 487 | RVSN | 150 | LQSSHFP | 151 |
| SANSPTM_Y | TTYFN | | RFS | | WT | |
| CD3_sp11a_VHVL_TT_SA | RSSQSLVRSEG | 487 | RVSN | 150 | LQSSHFP | 151 |
| NSPTM_S | TTYFN | | RFS | | WT | |
| CD3_sp11a_VHVL_TT_Y | RSSQSLVRSDG | 149 | RVSN | 150 | LQSSHFP | 151 |
| | TTYFN | | RFS | | WT | |
| CD3_sp11a_VHVL_TT_S | RSSQSLVRSDG | 149 | RVSN | 150 | LQSSHFP | 151 |
| | TTYFN | | RFS | | WT | |
| CD3_sp11a_VHVL_TT_SA | RSSQSLVRSEG | 487 | RVSN | 150 | LQSSHFP | 151 |
| NSPTM | TTYFN | | RFS | | WT | |
| CD3_SP11AVH3_VLK_3_Y | RSSQSLVRSEG | 487 | RVSN | 150 | LQSSHFP | 151 |
| | TTYFN | | RFS | | WT | |
| CD3_SP11AVH3_VLK_3_S | RSSQSLVRSEG | 487 | RVSN | 150 | LQSSHFP | 151 |
| | TTYFN | | RFS | | WT | |
| CD3_SP11AVH3_VLK_3_Y_ | RSSQSLVRSDG | 149 | RVSN | 150 | LQSSHFP | 151 |
| PTM | TTYFN | | RFS | | WT | |
| CD3_SP11AVH3_VLK_3_S_ | RSSQSLVRSDG | 149 | RVSN | 150 | LQSSHFP | 151 |
| PTM | TTYFN | | RFS | | WT | |
| CD3_SP11AVH3_VLK_3_Y_ | RSSQSLVRSEG | 487 | RVSN | 150 | LQSSHFP | 151 |
| SW | TTYFN | | RFS | | WT | |
| CD3_SP11AVH3_VLK_3_S_ | RSSQSLVRSEG | 487 | RVSN | 150 | LQSSHFP | 151 |
| SW | TTYFN | | RFS | | WT | |
| CD3_SP11AVH3_VLK_3_Y_ | RSSQSLVRSDG | 149 | RVSN | 150 | LQSSHFP | 151 |
| PTM_SW | TTYFN | | RFS | | WT | |
| CD3_SP11AVH3_VLK_3_S_ | RSSQSLVRSDG | 149 | RVSN | 150 | LQSSHFP | 151 |
| SVVPTM | TTYFN | | RFS | | WT | |
| CD3_SP11AVH3_VLK_SW | RSSQSLVRSDG | 149 | RVSN | 150 | LQSSHFP | 151 |
| PTM | TTYFN | | RFS | | WT | |
| CD3_SP11AVH3_VLK_3_S | RSSQSLVRSEG | 487 | RVSN | 150 | LQSSHFP | 151 |
| W | TTYFN | | RFS | | WT | |
| CD3_sp11a_VH1_VK2_Y | RSSQSLVRSDG | 149 | RVSN | 150 | LQSSHFP | 151 |
| | TTYFN | | RFS | | WT | |
| CD3_sp11a_VH1_VK2_S | RSSQSLVRSDG | 149 | RVSN | 150 | LQSSHFP | 151 |
| | TTYFN | | RFS | | WT | |
| CD3_sp11a_VH1_VK2_Y_P | RSSQSLVRSEG | 487 | RVSN | 150 | LQSSHFP | 151 |
| TM | TTYFN | | RFS | | WT | |
| CD3_sp11a_VH1_VK2_S_P | RSSQSLVRSEG | 487 | RVSN | 150 | LQSSHFP | 151 |
| TM | TTYFN | | RFS | | WT | |
| CD3_sp11a_VH1_VK2_Y_SW | RSSQSLVRSDG | 149 | RVSN | 150 | LQSSHFP | 151 |
| | TTYFN | | RFS | | WT | |
| CD3_sp11a_VH1_VK2_S_SW | RSSQSLVRSDG | 149 | RVSN | 150 | LQSSHFP | 151 |
| | TTYFN | | RFS | | WT | |
| CD3_sp11a_VH1_VK2_Y_P | RSSQSLVRSEG | 487 | RVSN | 150 | LQSSHFP | 151 |
| TM | TTYFN | | RFS | | WT | |
| CD3_sp11a_VH1_VK2_S_P | RSSQSLVRSEG | 487 | RVSN | 150 | LQSSHFP | 151 |
| TM_SW | TTYFN | | RFS | | WT | |
| CD3_sp11a_VH1_VK2_SW | RSSQSLVRSDG | 149 | RVSN | 150 | LQSSHFP | 151 |
| | TTYFN | | RFS | | WT | |
| CD3_sp11a_VH1_VK2_SW_ | RSSQSLVRSEG | 487 | RVSN | 150 | LQSSHFP | 151 |
| PTM | TTYFN | | RFS | | WT | |
| CD3_SP11A_VH3_VLK1_Y | RSSQSLVRSEG | 487 | RVSN | 150 | LQSSHFP | 151 |
| | TTYFN | | RFS | | WT | |
| CD3_SP11A_VH3_VLK1_S | RSSQSLVRSEG | 487 | RVSN | 150 | LQSSHFP | 151 |
| | TTYFN | | RFS | | WT | |
| CD3_SP11A_VH3_VLK1_Y_ | RSSQSLVRSEG | 487 | RVSN | 150 | LQSSHFP | 151 |
| PTM | TTYFN | | RFS | | WT | |
| CD3_SP11A_VH3_VLK1_S_ | RSSQSLVRSDG | 149 | RVSN | 150 | LQSSHFP | 151 |
| PTM | TTYFN | | RFS | | WT | |
| CD3_SP11A_VH3_VLK1_Y_ | RSSQSLVRSEG | 487 | RVSN | 150 | LQSSHFP | 151 |
| SW | TTYFN | | RFS | | WT | |
| CD3_SP11A_VH3_VLK1_S_ | RSSQSLVRSEG | 487 | RVSN | 150 | LQSSHFP | 151 |
| SW | TTYFN | | RFS | | WT | |
| CD3_SP11A_VH3_VLK1_Y_ | RSSQSLVRSEG | 487 | RVSN | 150 | LQSSHFP | 151 |
| PTM | TTYFN | | RFS | | WT | |
| CD3_SP11A_VH3_VLK1_S_ | RSSQSLVRSDG | 149 | RVSN | 150 | LQSSHFP | 151 |
| PTM_SW | TTYFN | | RFS | | WT | |
| CD3_SP11A_VH3_VLK1PT | RSSQSLVRSDG | 149 | RVSN | 150 | LQSSHFP | 151 |
| M_SW | TTYFN | | RFS | | WT | |
| CD3_SP11A_VH3_VLK1_S | RSSQSLVRSEG | 487 | RVSN | 150 | LQSSHFP | 151 |
| W | TTYFN | | RFS | | WT | |
| CD3_SP11A_VH5_VK2_Y | RSSQSLVRSDG | 149 | RVSN | 150 | LQSSHFP | 151 |
| | TTYFN | | RFS | | WT | |
| CD3_SP11A_VH5_VK2_S | RSSQSLVRSDG | 149 | RVSN | 150 | LQSSHFP | 151 |
| | TTYFN | | RFS | | WT | |
| CD3_SP11A_VH5_VK2_Y_ | RSSQSLVRSDG | 149 | RVSN | 150 | LQSSHFP | 151 |
| PTM | TTYFN | | RFS | | WT | |
| CD3_SP11A_VH5_VK2_S_ | RSSQSLVRSDG | 149 | RVSN | 150 | LQSSHFP | 151 |
| PTM | TTYFN | | RFS | | WT | |
| CD3_SP11A_VH5_VK2_Y_ | RSSQSLVRSDG | 149 | RVSN | 150 | LQSSHFP | 151 |
| SW | TTYFN | | RFS | | WT | |
| CD3_SP11A_VH5_VK2_S_ | RSSQSLVRSDG | 149 | RVSN | 150 | LQSSHFP | 151 |
| SW | TTYFN | | RFS | | WT | |
| CD3_SP11A_VH5_VK2_Y_ | RSSQSLVRSDG | 149 | RVSN | 150 | LQSSHFP | 151 |
| PTM_SW | TTYFN | | RFS | | WT | |
| CD3_SP11A_VH5_VK2_S_ | RSSQSLVRSDG | 149 | RVSN | 150 | LQSSHFP | 151 |
| PTM_SW | TTYFN | | RFS | | WT | |
| CD3_SP11A_VH5_VK2_PT | RSSQSLVRSDG | 149 | RVSN | 150 | LQSSHFP | 151 |
| M_SW | TTYFN | | RFS | | WT | |
| CD3_SP11A_VH5_VK2_SW | RSSQSLVRSDG | 149 | RVSN | 150 | LQSSHFP | 151 |
| | TTYFN | | RFS | | WT | |

TABLE 1H-1
CD3 Binders - Heavy Chain CDR sequences according to combination of Kabat and
IMGT numbering schemes

| | | SEQ ID | | SEQ ID | | SEQ ID |
|---|---|---|---|---|---|---|
| Binder | CDR-H1 | NO: | CDR-H2 | NO: | CDR-H3 | NO: |
| NOV292 | GFTFSK | 133 | MIYYDSSKMY | 134 | ASFVWVDLDFD | 144 |
| | NGMH | | YADTVKG | | H | |
| NOV123 | GYTFTS | 165 | YIYPGHDAIYY | 166 | VRPNTMMAPL | 176 |
| | YYIY | | SENFKG | | AY | |
| Sp10b | GYTFTS | 165 | YIYPGHDAIYY | 166 | VRPNTMMAPL | 176 |
| | YYIY | | SENFKG | | AY | |
| NOV453 | GFSLTT | 197 | RMRYSGDTSF | 198 | TSDPMYIPNYS | 208 |
| | YNVH | | NAALTS | | YGVMNA | |
| NOV229 | GFSLTT | 197 | RMRYSGDTSF | 198 | ARDPMYIPNYS | 240 |
| | YNVH | | NAALTS | | YGVMNA | |
| NOV110 | GYTFTS | 165 | YIYPANGGIYY | 262 | ARPVTMMAPL | 272 |
| | YYIY | | SEKFKG | | VF | |
| NOV832 | GYTFTS | 165 | YIYPANGGIYY | 262 | ARPVTMMAPL | 272 |
| | YYIY | | SEKFKG | | VF | |
| NOV589 | GFTFSK | 133 | MIYYDSSRMY | 326 | ASFVWVDLDFD | 336 |
| | NGMH | | YADTVKG | | Y | |
| NOV580 | GFSLTT | 357 | RMRYSGDTSY | 358 | TRDPMYIPGYS | 368 |
| | YNIH | | SSALKS | | YGVMNA | |
| NOV567 | GFAFRK | 389 | LIYYDSSKMNY | 390 | AALNSEYD | 400 |
| | YGMS | | ADTVKG | | | |
| NOV221 | GFSLTT | 357 | RMRYSGDTSY | 358 | TRDPMYIPGYS | 368 |
| | YNIH | | SSALKS | | YGVMNA | |
| CD3_sp11a_bkm1 | GFTFSK | 133 | MIYYDSSKMY | 134 | ASFVWVDLDFD | 144 |
| | NGMH | | YADTVKG | | H | |
| CD3_SP11a_bkm2 | GFTFSK | 133 | MIYYDSSKMY | 134 | AKFVWVDLDFD | 462 |
| | NGMH | | YADTVKG | | H | |
| CD3_sp11a_hz0 | GFTFSK | 133 | MIYYDSSKMY | 134 | AKFVWVDLDFD | 462 |
| | NGMH | | YADTVKG | | H | |
| CD3_SP11A_HZ1 | GFTFSK | 133 | MIYYDSSKMY | 134 | ASFVWVDLDFD | 144 |
| | NGMH | | YADTVKG | | H | |
| CD3_sp11a_sansPTM_ | GFTFSK | 481 | MIYYDSSKMY | 134 | ASFVWVDLDFD | 144 |
| hz1 | QGMH | | YADTVKG | | H | |
| CD3_sp11a_sansPTM_ | GFTFSK | 481 | MIYYDSSKMY | 134 | ASFVWVDLDFD | 144 |
| rat | QGMH | | YADTVKG | | H | |
| CD3_sp11a_VHVL_YY | GFTFSK | 133 | MIYYDSSKMY | 134 | ASFYYDLDFD | 470 |
| | NGMH | | YADTVKG | | H | |
| CD3_SP11A_VHVL_SS | GFTFSK | 133 | MIYYDSSKMY | 134 | ASFSSDLDFD | 464 |
| | NGMH | | YADTVKG | | H | |
| CD3_SP11A_VHVL_W | GFTFSK | 133 | MIYYDSSKMY | 134 | ASFWSDLDFD | 468 |
| S | NGMH | | YADTVKG | | H | |
| CD3_sp11a_VHVL_SW | GFTFSK | 133 | MIYYDSSKMY | 134 | ASFSVVDLDFD | 465 |
| NGMH | YADTVKG | H | | | | |
| CD3_SP11A_VHVL_TT | GFTFSK | 133 | MIYYDSSKMY | 134 | ASFTTDLDFDH | 466 |
| | NGMH | | YADTVKG | | | |
| CD3_SP11A_VHVL_T | GFTFSK | 133 | MIYYDSSKMY | 134 | ASFTWDLDFD | 467 |
| W | NGMH | | YADTVKG | | H | |
| CD3_SP11A_VHVL_W | GFTFSK | 133 | MIYYDSSKMY | 134 | ASFVVTDLDFD | 469 |
| T | NGMH | | YADTVKG | | H | |
| CD3_SP11A | GFTFSK | 133 | MIYYDSSKMY | 134 | ASFVWVDLDFD | 144 |
| VH3_VLK_3 | NGMH | | YADTVKG | | H | |
| CD3_sp11a_VH1_VK2 | GFTFSK | 481 | MIYYDSSKMY | 134 | ASFVWVDLDFD | 144 |
| | QGMH | | YADTVKG | | H | |
| CD3_SP11A_VH3_VLK1 | GFTFSK | 133 | MIYYDSSKMY | 134 | ASFVWVDLDFD | 144 |
| | NGMH | | YADTVKG | | H | |
| CD3_SP11A_VH5_VK2 | GFTFSK | 481 | MIYYDSSKMY | 134 | ASFVWVDLDFD | 144 |
| | QGMH | | YADTVKG | | H | |
| CD3_sp9aFW1_VL_VH_ | GFSLTT | 197 | RMRYSGDTSF | 198 | ASDPMYIPNYA | 463 |
| S56G | YNVH | | NAALTS | | YGVMNA | |
| CD3_SP9AFW4_VL_V | GFSLTT | 197 | RMRYSGDTSF | 198 | ASDPMYIPNYA | 463 |
| H_S56G | YNVH | | NAALTS | | YGVMNA | |
| CD3_sp9aFW1_VLVH | GFSLTT | 197 | RMRYSGDTSF | 198 | ASDPMYIPNYA | 463 |
| | YNVH | | NAALTS | | YGVMNA | |
| CD3_sp9aFW4_VLVH | GFSLTT | 197 | RMRYSGDTSF | 198 | ASDPMYIPNYA | 463 |
| | YNVH | | NAALTS | | YGVMNA | |
| CD3_sp9arabtor_VHVL | GFSLTT | 197 | RMRYSGDTSF | 198 | ASDPMYIPNYA | 463 |
| | YNVH | | NAALTS | | YGVMNA | |
| CD3_sp9arabtor_VLVH | GFSLTT | 197 | RMRYSGDTSF | 198 | ASDPMYIPNYA | 463 |
| | YNVH | | NAALTS | | YGVMNA | |
| CD3_SP11AVH3_VLK_ | GFTFSK | 133 | MIYYDSSKMY | 134 | ASYVWVDLDFD | 553 |
| 3_Y | NGMH | | YADTVKG | | H | |
| CD3_SP11AVH3_VLK_ | GFTFSK | 133 | MIYYDSSKMY | 134 | ASSVWVDLDFD | 546 |
| 3_S | NGMH | | YADTVKG | | H | |
| CD3_SP11AVH3_VLK_ | GFTFSK | 133 | MIYYDSSKMY | 134 | ASYVWVDLDFD | 553 |
| 3_Y_PTM | NGMH | | YADTVKG | | H | |
| CD3_SP11AVH3_VLK_ | GFTFSK | 133 | MIYYDSSKMY | 134 | ASSVWVDLDFD | 546 |
| 3_S_PTM | NGMH | | YADTVKG | | H | |
| CD3_SP11AVH3_VLK_ | GFTFSK | 133 | MIYYDSSKMY | 134 | ASYSVVDLDFD | 549 |
| 3_Y_SW | NGMH | | YADTVKG | | H | |
| CD3_SP11AVH3_VLK_ | GFTFSK | 133 | MIYYDSSKMY | 134 | ASSSVVDLDFD | 542 |
| 3_S_SW | NGMH | | YADTVKG | | H | |
| CD3_SP11AVH3_VLK_ | GFTFSK | 133 | MIYYDSSKMY | 134 | ASYSVVDLDFD | 549 |
| 3_Y_PTM_SW | NGMH | | YADTVKG | | H | |
| CD3_SP11AVH3_VLK_ | GFTFSK | 133 | MIYYDSSKMY | 134 | ASSSVVDLDFD | 542 |
| 3_S_SVVPTM | NGMH | | YADTVKG | | H | |
| CD3_SP11AVH3_VLK_ | GFTFSK | 133 | MIYYDSSKMY | 134 | ASFSVVDLDFD | 465 |
| SVVPTM | NGMH | | YADTVKG | | H | |
| CD3_SP11AVH3_VLK_ | GFTFSK | 133 | MIYYDSSKMY | 134 | ASFSVVDLDFD | 465 |
| 3_SW | NGMH | | YADTVKG | | H | |
| CD3_sp11a_VH1_VK2_ | GFTFSK | 481 | MIYYDSSKMY | 134 | ASYVWVDLDFD | 553 |
| Y | QGMH | | YADTVKG | | H | |
| CD3_sp11a_VH1_VK2_ | GFTFSK | 481 | MIYYDSSKMY | 134 | ASSVWVDLDFD | 546 |
| S | QGMH | | YADTVKG | | H | |
| CD3_sp11a_VH1_VK2_ | GFTFSK | 133 | MIYYDSSKMY | 134 | ASYVWVDLDFD | 553 |
| Y_PTM | NGMH | | YADTVKG | | H | |
| CD3_sp11a_VH1_VK2_ | GFTFSK | 133 | MIYYDSSKMY | 134 | ASSVWVDLDFD | 546 |
| S_PTM | NGMH | | YADTVKG | | H | |
| CD3_sp11a_VH1_VK2_ | GFTFSK | 481 | MIYYDSSKMY | 134 | ASYSVVDLDFD | 549 |
| Y_SW | QGMH | | YADTVKG | | H | |
| CD3_sp11a_VH1_VK2_ | GFTFSK | 481 | MIYYDSSKMY | 134 | ASSSVVDLDFD | 542 |
| S_SW | QGMH | | YADTVKG | | H | |
| CD3_sp11a_VH1_VK2_ | GFTFSK | 133 | MIYYDSSKMY | 134 | ASYSVVDLDFD | 549 |
| Y_PTM | NGMH | | YADTVKG | | H | |
| CD3_sp11a_VH1_VK2_ | GFTFSK | 133 | MIYYDSSKMY | 134 | ASSSVVDLDFD | 542 |
| S_PTM_SW | NGMH | | YADTVKG | | H | |
| CD3 sp11a_VH1_VK2_ | GFTFSK | 481 | MIYYDSSKMY | 134 | ASFSVVDLDFD | 465 |
| SW | QGMH | | YADTVKG | | H | |
| CD3 sp11a_VH1_VK2_ | GFTFSK | 133 | MIYYDSSKMY | 134 | ASFSVVDLDFD | 465 |
| SW_PTM | NGMH | | YADTVKG | | H | |
| CD3_SP11A_VH3_VLK | GFTFSK | 133 | MIYYDSSKMY | 134 | ASYVWVDLDFD | 553 |
| 1_Y | NGMH | | YADTVKG | | H | |
| CD3_SP11A_VH3_VLK | GFTFSK | 133 | MIYYDSSKMY | 134 | ASSVWVDLDFD | 546 |
| 1_S | NGMH | | YADTVKG | | H | |
| CD3_SP11A_VH3_VLK | GFTFSK | 481 | MIYYDSSKMY | 134 | ASYVWVDLDFD | 553 |
| 1_Y_PTM | QGMH | | YADTVKG | | H | |
| CD3_SP11A_VH3_VLK | GFTFSK | 481 | MIYYDSSKMY | 134 | ASSVWVDLDFD | 546 |
| 1_S_PTM | QGMH | | YADTVKG | | H | |
| CD3_SP11A_VH3_VLK | GFTFSK | 133 | MIYYDSSKMY | 134 | ASYSVVDLDFD | 549 |
| 1_Y_SW | NGMH | | YADTVKG | | H | |
| CD3_SP11A_VH3_VLK | GFTFSK | 133 | MIYYDSSKMY | 134 | ASSSVVDLDFD | 542 |
| 1_S_SW | NGMH | | YADTVKG | | H | |
| CD3_SP11A_VH3_VLK | GFTFSK | 481 | MIYYDSSKMY | 134 | ASYVWVDLDFD | 553 |
| 1_Y_PTM | QGMH | | YADTVKG | | H | |
| CD3_SP11A_VH3_VLK | GFTFSK | 481 | MIYYDSSKMY | 134 | ASSSVVDLDFD | 542 |
| 1_S_PTM_SW | QGMH | | YADTVKG | | H | |
| CD3_SP11A_VH3_VLK | GFTFSK | 481 | MIYYDSSKMY | 134 | ASFSVVDLDFD | 465 |
| 1PTM_SW | QGMH | | YADTVKG | | H | |
| CD3_SP11A_VH3_VLK | GFTFSK | 133 | MIYYDSSKMY | 134 | ASFSVVDLDFD | 465 |
| 1_SW | NGMH | | YADTVKG | | H | |
| CD3_SP11A_VH5_VK2_ | GFTFSK | 481 | MIYYDSSKMY | 134 | ASYVWVDLDFD | 553 |
| Y | QGMH | | YADTVKG | | H | |
| CD3_SP11A_VH5_VK2_ | GFTFSK | 481 | MIYYDSSKMY | 134 | ASSVWVDLDFD | 546 |
| S | QGMH | | YADTVKG | | H | |
| CD3_SP11A_VH5_VK2_ | GFTFSK | 133 | MIYYDSSKMY | 134 | ASYVWVDLDFD | 553 |
| Y_PTM | NGMH | | YADTVKG | | H | |
| CD3_SP11A_VH5_VK2_ | GFTFSK | 133 | MIYYDSSKMY | 134 | ASSVWVDLDFD | 546 |
| S_PTM | NGMH | | YADTVKG | | H | |
| CD3_SP11A_VH5_VK2_ | GFTFSK | 481 | MIYYDSSKMY | 134 | ASYSVVDLDFD | 549 |
| Y_SW | QGMH | | YADTVKG | | H | |
| CD3_SP11A_VH5_VK2_ | GFTFSK | 481 | MIYYDSSKMY | 134 | ASSSVVDLDFD | 542 |
| S_SW | QGMH | | YADTVKG | | H | |
| CD3_SP11A_VH5_VK2_ | GFTFSK | 133 | MIYYDSSKMY | 134 | ASYSVVDLDFD | 549 |
| Y_PTM_SW | NGMH | | YADTVKG | | H | |
| CD3_SP11A_VH5_VK2_ | GFTFSK | 133 | MIYYDSSKMY | 134 | ASSSVVDLDFD | 542 |
| S_PTM_SW | NGMH | | YADTVKG | | H | |
| CD3_SP11A_VH5_VK2_ | GFTFSK | 133 | MIYYDSSKMY | 134 | ASFSVVDLDFD | 465 |
| PTM_SW | NGMH | | YADTVKG | | H | |
| CD3_SP11A_VH5_VK2_ | GFTFSK | 481 | MIYYDSSKMY | 134 | ASFSVVDLDFD | 465 |
| SW | QGMH | | YADTVKG | | H | |

TABLE 1H-2
CD3 Binders-Light Chain CDR sequences according to combination of Kabat and
MGT numbering schemes

| | | SEQ ID | | SEQ ID | | SEQ ID |
|---|---|---|---|---|---|---|
| Binder | CDR-L1 | NO: | CDR-L2 | NO: | CDR-L3 | NO: |
| NOV292 | RSSQSLVRSD | 149 | RVSNRFS | 150 | LQSSHF | 151 |
| | GTTYFN | | | | PWT | |
| NOV123 | RSSQSLIYSIGN | 181 | RVSNRFS | 150 | FQSTHL | 183 |
| | TYLH | | | | PYT | |
| Sp10b | RSSQSLIYSIGN | 181 | RVSNRFS | 150 | FQSTHL | 183 |
| | TYLH | | | | PYT | |
| NOV453 | KASQNINNYLN | 213 | NTDHLQA | 223 | LQHRSR | 215 |
| | | | GVP | | YT | |
| NOV229 | KASQNINNYLN | 213 | NTDHLQA | 223 | LQHRSR | 215 |
| | | | GVP | | YT | |
| NOV110 | RSSQSLVYSH | 277 | RVSNRFS | 150 | FQSTHL | 183 |
| | GNTYLH | | | | PYT | |
| NOV832 | RSSQSLVYSH | 277 | RVSNRFS | 150 | FQSTHL | 183 |
| | GNTYLH | | | | PYT | |
| NOV589 | RSSQSLVRSD | 149 | RVSNRFS | 150 | LQSSHF | 151 |
| | GTTYFN | | | | PWT | |
| NOV580 | KTSQNIDKYLN | 373 | NTNNLEA | 383 | LQHRSS | 375 |
| | | | GVP | | YT | |
| NOV567 | RGSQSIGNSLN | 405 | STSTLEY | 415 | LQYATY | 407 |
| | | | GVP | | PYT | |
| NOV221 | KSSQNIDKYLN | 437 | NTNNLEA | 383 | LQHRSG | 439 |
| | | | GVP | | YT | |
| CD3_sp11a_bkm1 | RSSQSLVRSD | 149 | RVSNRFS | 150 | LQSSHF | 151 |
| | GTTYFN | | | | PWT | |
| CD3_SP11a_bkm2 | RSSQSLVRSD | 149 | RVSNRFS | 150 | LQSSHF | 151 |
| | GTTYFN | | | | PWT | |
| CD3_sp11a_hz0 | RSSQSLVRSD | 149 | RVSNRFS | 150 | LQSSHF | 151 |
| | GTTYFN | | | | PWT | |
| CD3_SP11A_HZ1 | RSSQSLVRSD | 149 | RVSNRFS | 150 | LQSSH | 484 |
| | GTTYFN | | | | | |
| CD3_sp11a_sansPTM_hz1 | RSSQSLVRSE | 487 | RVSNRFS | 150 | LQSSHF | 151 |
| | GTTYFN | | | | PWT | |
| CD3_sp11a_sansPTM_rat | RSSQSLVRSE | 487 | RVSNRFS | 150 | LQSSHF | 151 |
| | GTTYFN | | | | PWT | |
| CD3_sp11a_VHVL_YY | RSSQSLVRSD | 149 | RVSNRFS | 150 | LQSSHF | 151 |
| | GTTYFN | | | | PWT | |
| CD3_SP11A_VHVL_SS | RSSQSLVRSD | 149 | RVSNRFS | 150 | LQSSHF | 151 |
| | GTTYFN | | | | PWT | |
| CD3_SP11A_VHVL_WS | RSSQSLVRSD | 149 | RVSNRFS | 150 | LQSSHF | 151 |
| | GTTYFN | | | | PWT | |
| CD3_sp11a_VHVL_SW | RSSQSLVRSD | 149 | RVSNRFS | 150 | LQSSHF | 151 |
| | GTTYFN | | | | PWT | |
| CD3_SP11A_VHVL_TT | RSSQSLVRSD | 149 | RVSNRFS | 150 | LQSSHF | 151 |
| | GTTYFN | | | | PWT | |
| CD3_SP11A_VHVL_TW | RSSQSLVRSD | 149 | RVSNRFS | 150 | LQSSHF | 151 |
| | GTTYFN | | | | PWT | |
| CD3_SP11A_VHVL_VVT | RSSQSLVRSD | 149 | RVSNRFS | 150 | LQSSHF | 151 |
| | GTTYFN | | | | PWT | |
| CD3_SP11A VH3_VLK_3 | RSSQSLVRSE | 487 | RVSNRFS | 150 | LQSSHF | 151 |
| | GTTYFN | | | | PWT | |
| CD3_sp11a_VH1_VK2 | RSSQSLVRSD | 149 | RVSNRFS | 150 | LQSSHF | 151 |
| | GTTYFN | | | | PWT | |
| CD3_SP11A_VH3_VLK1 | RSSQSLVRSE | 487 | RVSNRFS | 150 | LQSSHF | 151 |
| | GTTYFN | | | | PWT | |
| CD3_SP11A_VH5_VK2 | RSSQSLVRSD | 149 | RVSNRFS | 150 | LQSSHF | 151 |
| | GTTYFN | | | | PWT | |
| CD3_sp9aFW1_VL_VH_S | KASQNINNYLN | 213 | NTDHLQA | 223 | LQHRSR | 215 |
| 56G | | | GVP | | YT | |
| CD3_SP9AFW4_VL_VH_ | KASQNINNYLN | 213 | NTDHLQA | 223 | LQHRSR | 215 |
| S56G | | | GVP | | YT | |
| CD3_sp9aFW1_VLVH | KASQNINNYLN | 213 | NTDHLQA | 223 | LQHRSR | 215 |
| | | | GVP | | YT | |
| CD3_sp9aFW4_VLVH | KASQNINNYLN | 213 | NTDHLQA | 223 | LQHRSR | 215 |
| | | | GVP | | YT | |
| CD3_sp9arabtor_VHVL | KASQNINNYLN | 213 | NTDHLQA | 223 | LQHRSR | 215 |
| | | | GVP | | YT | |
| CD3_sp9arabtor_VLVH | KASQNINNYLN | 213 | NTDHLQA | 223 | LQHRSR | 215 |
| | | | GVP | | YT | |
| CD3_SP11AVH3_VLK_3_Y | RSSQSLVRSE | 487 | RVSNRFS | 150 | LQSSHF | 151 |
| | GTTYFN | | | | PWT | |
| CD3_SP11AVH3_VLK_3_S | RSSQSLVRSE | 487 | RVSNRFS | 150 | LQSSHF | 151 |
| | GTTYFN | | | | PWT | |
| CD3_SP11AVH3_VLK_3_ | RSSQSLVRSD | 149 | RVSNRFS | 150 | LQSSHF | 151 |
| Y_PTM | GTTYFN | | | | PWT | |
| CD3_SP11AVH3_VLK_3_ | RSSQSLVRSD | 149 | RVSNRFS | 150 | LQSSHF | 151 |
| S_PTM | GTTYFN | | | | PWT | |
| CD3_SP11AVH3_VLK_3_ | RSSQSLVRSE | 487 | RVSNRFS | 150 | LQSSHF | 151 |
| Y_SW | GTTYFN | | | | PWT | |
| CD3_SP11AVH3_VLK_3_ | RSSQSLVRSE | 487 | RVSNRFS | 150 | LQSSHF | 151 |
| S_SW | GTTYFN | | | | PWT | |
| CD3_SP11AVH3_VLK_3_ | RSSQSLVRSD | 149 | RVSNRFS | 150 | LQSSHF | 151 |
| Y_PTM_SW | GTTYFN | | | | PWT | |
| CD3_SP11AVH3_VLK_3_ | RSSQSLVRSD | 149 | RVSNRFS | 150 | LQSSHF | 151 |
| S_SWPTM | GTTYFN | | | | PWT | |
| CD3_SP11AVH3_VLK_S | RSSQSLVRSD | 149 | RVSNRFS | 150 | LQSSHF | 151 |
| VVPTM | GTTYFN | | | | PWT | |
| CD3_SP11AVH3_VLK_3_ | RSSQSLVRSE | 487 | RVSNRFS | 150 | LQSSHF | 151 |
| SW | GTTYFN | | | | PWT | |
| CD3_sp11a_VH1_VK2_Y | RSSQSLVRSD | 149 | RVSNRFS | 150 | LQSSHF | 151 |
| | GTTYFN | | | | PWT | |
| CD3_sp11a_VH1_VK2_S | RSSQSLVRSD | 149 | RVSNRFS | 150 | LQSSHF | 151 |
| | GTTYFN | | | | PWT | |
| CD3_sp11a_VH1_VK2_Y_ | RSSQSLVRSE | 487 | RVSNRFS | 150 | LQSSHF | 151 |
| PTM | GTTYFN | | | | PWT | |
| CD3_sp11a_VH1_VK2_S_ | RSSQSLVRSE | 487 | RVSNRFS | 150 | LQSSHF | 151 |
| PTM | GTTYFN | | | | PWT | |
| CD3_sp11a_VH1_VK2_Y_ | RSSQSLVRSD | 149 | RVSNRFS | 150 | LQSSHF | 151 |
| SW | GTTYFN | | | | PWT | |
| CD3_sp11a_VH1_VK2_S_ | RSSQSLVRSD | 149 | RVSNRFS | 150 | LQSSHF | 151 |
| SW | GTTYFN | | | | PWT | |
| CD3_sp11a_VH1_VK2_Y_ | RSSQSLVRSE | 487 | RVSNRFS | 150 | LQSSHF | 151 |
| PTM | GTTYFN | | | | PWT | |
| CD3_sp11a_VH1_VK2_S_ | RSSQSLVRSE | 487 | RVSNRFS | 150 | LQSSHF | 151 |
| PTM_SW | GTTYFN | | | | PWT | |
| CD3_sp11a_VH1_VK2_S | RSSQSLVRSD | 149 | RVSNRFS | 150 | LQSSHF | 151 |
| W | GTTYFN | | | | PWT | |
| CD3_sp11a_VH1_VK2_ | RSSQSLVRSE | 487 | RVSNRFS | 150 | LQSSHF | 151 |
| SWPTM | GTTYFN | | | | PWT | |
| CD3_SP11A_VH3_VLK1_Y | RSSQSLVRSE | 487 | RVSNRFS | 150 | LQSSHF | 151 |
| | GTTYFN | | | | PWT | |
| CD3_SP11A_VH3_VLK1_S | RSSQSLVRSE | 487 | RVSNRFS | 150 | LQSSHF | 151 |
| | GTTYFN | | | | PWT | |
| CD3_SP11A_VH3_VLK1_ | RSSQSLVRSD | 149 | RVSNRFS | 150 | LQSSHF | 151 |
| Y_PTM | GTTYFN | | | | PWT | |
| CD3_SP11A_VH3_VLK1_ | RSSQSLVRSD | 149 | RVSNRFS | 150 | LQSSHF | 151 |
| S_PTM | GTTYFN | | | | PWT | |
| CD3_SP11A_VH3_VLK1_ | RSSQSLVRSE | 487 | RVSNRFS | 150 | LQSSHF | 151 |
| Y_SW | GTTYFN | | | | PWT | |
| CD3_SP11A_VH3_VLK1_ | RSSQSLVRSE | 487 | RVSNRFS | 150 | LQSSHF | 151 |
| S_SW | GTTYFN | | | | PWT | |
| CD3_SP11A_VH3_VLK1_ | RSSQSLVRSD | 149 | RVSNRFS | 150 | LQSSHF | 151 |
| Y_PTM | GTTYFN | | | | PWT | |
| CD3_SP11A_VH3_VLK1_ | RSSQSLVRSD | 149 | RVSNRFS | 150 | LQSSHF | 151 |
| S_PTM_SW | GTTYFN | | | | PWT | |
| CD3_SP11A_VH3_VLK1P | RSSQSLVRSD | 149 | RVSNRFS | 150 | LQSSHF | 151 |
| TM_SW | GTTYFN | | | | PWT | |
| CD3_SP11A_VH3_VLK1_ | RSSQSLVRSE | 487 | RVSNRFS | 150 | LQSSHF | 151 |
| SW | GTTYFN | | | | PWT | |
| CD3_SP11A_VH5_VK2_Y | RSSQSLVRSD | 149 | RVSNRFS | 150 | LQSSHF | 151 |
| | GTTYFN | | | | PWT | |
| CD3_SP11A_VH5_VK2_S | RSSQSLVRSD | 149 | RVSNRFS | 150 | LQSSHF | 151 |
| | GTTYFN | | | | PWT | |
| CD3_SP11A_VH5_VK2_Y_ | RSSQSLVRSD | 149 | RVSNRFS | 150 | LQSSHF | 151 |
| PTM | GTTYFN | | | | PWT | |
| CD3_SP11A_VH5_VK2_S_ | RSSQSLVRSD | 149 | RVSNRFS | 150 | LQSSHF | 151 |
| PTM | GTTYFN | | | | PWT | |
| CD3_SP11A_VH5_VK2_Y_ | RSSQSLVRSD | 149 | RVSNRFS | 150 | LQSSHF | 151 |
| SW | GTTYFN | | | | PWT | |
| CD3_SP11A_VH5_VK2_S_ | RSSQSLVRSD | 149 | RVSNRFS | 150 | LQSSHF | 151 |
| SW | GTTYFN | | | | PWT | |
| CD3_SP11A_VH5_VK2_Y_ | RSSQSLVRSD | 149 | RVSNRFS | 150 | LQSSHF | 151 |
| PTM_SW | GTTYFN | | | | PWT | |
| CD3_SP11A_VH5_VK2_S_ | RSSQSLVRSD | 149 | RVSNRFS | 150 | LQSSHF | 151 |
| PTM_SW | GTTYFN | | | | PWT | |
| CD3_SP11A_VH5_VK2_P | RSSQSLVRSD | 149 | RVSNRFS | 150 | LQSSHF | 151 |
| TM_SW | GTTYFN | | | | PWT | |
| CD3_SP11A_VH5_VK2_S | RSSQSLVRSD | 149 | RVSNRFS | 150 | LQSSHF | 151 |
| W | GTTYFN | | | | PWT | |

TABLE 1I-1
CD3 Binders-Heavy Chain CDR sequences according to combination of Chothia
and IMGT numbering schemes

| | | SEQ ID | CDR- | SEQ ID | | SEQ ID |
|---|---|---|---|---|---|---|
| Binder | CDR-H1 | NO: | H2 | NO: | CDR-H3 | NO: |
| NOV292 | GFTFSK | 142 | YYDS | 140 | ASFVWVDLDFDH | 144 |
| | NG | | SK | | | |
| NOV123 | GYTFTS | 174 | YPGH | 172 | VRPNTMMAPLAY | 176 |
| | YY | | DA | | | |
| Sp10b | GYTFTS | 174 | YPGH | 172 | VRPNTMMAPLAY | 176 |
| | YY | | DA | | | |
| NOV453 | GFSLTT | 206 | RYSG | 204 | TSDPMYIPNYSY | 208 |
| | YN | | D | | GVMNA | |
| NOV229 | GFSLTT | 206 | RYSG | 204 | ARDPMYIPNYSY | 240 |
| | YN | | D | | GVMNA | |
| NOV110 | GYTFTS | 174 | YPAN | 268 | ARPVTMMAPLVF | 272 |
| | YY | | GG | | | |
| NOV832 | GYTFTS | 174 | YPAN | 268 | ARPVTMMAPLVF | 272 |
| | YY | | GG | | | |
| NOV589 | GFTFSK | 142 | YYDS | 332 | ASFVWVDLDFDY | 336 |
| | NG | | SR | | | |
| NOV580 | GFSLTT | 206 | RYSG | 204 | TRDPMYIPGYSY | 368 |
| | YN | | D | | GVMNA | |
| NOV567 | GFAFR | 398 | YYDS | 140 | AALNSEYD | 400 |
| | KYG | | SK | | | |
| NOV221 | GFSLTT | 206 | RYSG | 204 | TRDPMYIPGYSY | 368 |
| | YN | | D | | GVMNA | |
| CD3_sp11a_bkm1 | GFTFSK | 142 | YYDS | 140 | ASFVWVDLDFDH | 144 |
| | NG | | SK | | | |
| CD3_SP11a_bkm2 | GFTFSK | 142 | YYDS | 140 | AKFVWVDLDFDH | 462 |
| | NG | | SK | | | |
| CD3_sp11a_hz0 | GFTFSK | 142 | YYDS | 140 | AKFVWVDLDFDH | 462 |
| | NG | | SK | | | |
| CD3_SP11A_HZ1 | GFTFSK | 142 | YYDS | 140 | ASFVWVDLDFDH | 144 |
| | NG | | SK | | | |
| CD3_sp11a_sansPTM_hz1 | GFTFSK | 480 | YYDS | 140 | ASFVWVDLDFDH | 144 |
| | QG | | SK | | | |
| CD3_sp11a_sansPTM_rat | GFTFSK | 480 | YYDS | 140 | ASFVWVDLDFDH | 144 |
| | QG | | SK | | | |
| CD3_sp11a_VHVL_YY | GFTFSK | 142 | YYDS | 140 | ASFYYDLDFDH | 470 |
| | NG | | SK | | | |
| CD3_SP11A_VHVL_SS | GFTFSK | 142 | YYDS | 140 | ASFSSDLDFDH | 464 |
| | NG | | SK | | | |
| CD3_SP11A_VHVL_WS | GFTFSK | 142 | YYDS | 140 | ASFWSDLDFDH | 468 |
| | NG | | SK | | | |
| CD3_sp11a_VHVL_SW | GFTFSK | 142 | YYDS | 140 | ASFSVVDLDFDH | 465 |
| | NG | | SK | | | |
| CD3_SP11A_VHVL_TT | GFTFSK | 142 | YYDS | 140 | ASFTTDLDFDH | 466 |
| | NG | | SK | | | |
| CD3_SP11A_VHVL_TW | GFTFSK | 142 | YYDS | 140 | ASFTVVDLDFDH | 467 |
| | NG | | SK | | | |
| CD3_SP11A_VHVL_VVT | GFTFSK | 142 | YYDS | 140 | ASFVVTDLDFDH | 469 |
| | NG | | SK | | | |
| CD3_SP11A VH3_VLK_3 | GFTFSK | 142 | YYDS | 140 | ASFVWVDLDFDH | 144 |
| | NG | | SK | | | |
| CD3_sp11a_VH1_VK2 | GFTFSK | 480 | YYDS | 140 | ASFVWVDLDFDH | 144 |
| | QG | | SK | | | |
| CD3_SP11A_VH3_VLK1 | GFTFSK | 142 | YYDS | 140 | ASFVWVDLDFDH | 144 |
| | NG | | SK | | | |
| CD3_SP11A_VH5_VK2 | GFTFSK | 480 | YYDS | 140 | ASFVWVDLDFDH | 144 |
| | QG | | SK | | | |
| CD3 sp9aFW1_VL_VH_S5 | GFSLTT | 206 | RYSG | 204 | ASDPMYIPNYAY | 463 |
| 6G | YN | | D | | GVMNA | |
| CD3_SP9AFW4_VL_VH_S | GFSLTT | 206 | RYSG | 204 | ASDPMYIPNYAY | 463 |
| 56G | YN | | D | | GVMNA | |
| CD3_sp9aFW1_VLVH | GFSLTT | 206 | RYSG | 204 | ASDPMYIPNYAY | 463 |
| | YN | | D | | GVMNA | |
| CD3_sp9aFW4_VLVH | GFSLTT | 206 | RYSG | 204 | ASDPMYIPNYAY | 463 |
| | YN | | D | | GVMNA | |
| CD3_sp9arabtor_VHVL | GFSLTT | 206 | RYSG | 204 | ASDPMYIPNYAY | 463 |
| | YN | | D | | GVMNA | |
| CD3_sp9arabtor_VLVH | GFSLTT | 206 | RYSG | 204 | ASDPMYIPNYAY | 463 |
| | YN | | D | | GVMNA | |
| CD3_SP11AVH3_VLK_3_Y | GFTFSK | 142 | YYDS | 140 | ASYVWVDLDFDH | 553 |
| | NG | | SK | | | |
| CD3_SP11AVH3_VLK_3_S | GFTFSK | 142 | YYDS | 140 | ASSVWVDLDFDH | 546 |
| | NG | | SK | | | |
| CD3_SP11AVH3_VLK_3_Y_ | GFTFSK | 142 | YYDS | 140 | ASYVWVDLDFDH | 553 |
| PTM | NG | | SK | | | |
| CD3_SP11AVH3_VLK_3_S_ | GFTFSK | 142 | YYDS | 140 | ASSVWVDLDFDH | 546 |
| PTM | NG | | SK | | | |
| CD3_SP11AVH3_VLK_3_Y_ | GFTFSK | 142 | YYDS | 140 | ASYSVVDLDFDH | 549 |
| SW | NG | | SK | | | |
| CD3_SP11AVH3_VLK_3_S_ | GFTFSK | 142 | YYDS | 140 | ASSSVVDLDFDH | 542 |
| SW | NG | | SK | | | |
| CD3_SP11AVH3_VLK_3_Y_ | GFTFSK | 142 | YYDS | 140 | ASYSVVDLDFDH | 549 |
| PTM_SW | NG | | SK | | | |
| CD3_SP11AVH3_VLK_3_S_ | GFTFSK | 142 | YYDS | 140 | ASSSVVDLDFDH | 542 |
| SVVPTM | NG | | SK | | | |
| CD3_SP11AVH3_VLK_SW | GFTFSK | 142 | YYDS | 140 | ASFSVVDLDFDH | 465 |
| PTM | NG | | SK | | | |
| CD3_SP11AVH3_VLK_3_SW | GFTFSK | 142 | YYDS | 140 | ASFSVVDLDFDH | 465 |
| | NG | | SK | | | |
| CD3_sp11a_VH1_VK2_Y | GFTFSK | 480 | YYDS | 140 | ASYVWVDLDFDH | 553 |
| | QG | | SK | | | |
| CD3_sp11a_VH1_VK2_S | GFTFSK | 480 | YYDS | 140 | ASSVWVDLDFDH | 546 |
| | QG | | SK | | | |
| CD3_sp11a_VH1_VK2_Y_ | GFTFSK | 142 | YYDS | 140 | ASYVWVDLDFDH | 553 |
| PTM | NG | | SK | | | |
| CD3_sp11a_VH1_VK2_S_ | GFTFSK | 142 | YYDS | 140 | ASSVWVDLDFDH | 546 |
| PTM | NG | | SK | | | |
| CD3_sp11a_VH1_VK2_Y_ | GFTFSK | 480 | YYDS | 140 | ASYSVVDLDFDH | 549 |
| SW | QG | | SK | | | |
| CD3_sp11a_VH1_VK2_S_ | GFTFSK | 480 | YYDS | 140 | ASSSVVDLDFDH | 542 |
| SW | QG | | SK | | | |
| CD3_sp11a_VH1_VK2_Y_ | GFTFSK | 142 | YYDS | 140 | ASYSVVDLDFDH | 549 |
| PTM | NG | | SK | | | |
| CD3_sp11a_VH1_VK2_S_ | GFTFSK | 142 | YYDS | 140 | ASSSVVDLDFDH | 542 |
| PTM_SW | NG | | SK | | | |
| CD3_sp11a_VH1_VK2_SW | GFTFSK | 480 | YYDS | 140 | ASFSVVDLDFDH | 465 |
| | QG | | SK | | | |
| CD3_sp11a_VH1_VK2_SW_ | GFTFSK | 142 | YYDS | 140 | ASFSVVDLDFDH | 465 |
| PTM | NG | | SK | | | |
| CD3_SP11A_VH3_VLK1_Y | GFTFSK | 142 | YYDS | 140 | ASYVWVDLDFDH | 553 |
| | NG | | SK | | | |
| CD3_SP11A_VH3_VLK1_S | GFTFSK | 142 | YYDS | 140 | ASSVWVDLDFDH | 546 |
| | NG | | SK | | | |
| CD3_SP11A_VH3_VLK1_Y_ | GFTFSK | 480 | YYDS | 140 | ASYVWVDLDFDH | 553 |
| PTM | QG | | SK | | | |
| CD3_SP11A_VH3_VLK1_S_ | GFTFSK | 480 | YYDS | 140 | ASSVWVDLDFDH | 546 |
| PTM | QG | | SK | | | |
| CD3_SP11A_VH3_VLK1_Y_ | GFTFSK | 142 | YYDS | 140 | ASYSVVDLDFDH | 549 |
| SW | NG | | SK | | | |
| CD3_SP11A_VH3_VLK1_S_ | GFTFSK | 142 | YYDS | 140 | ASSSVVDLDFDH | 542 |
| SW | NG | | SK | | | |
| CD3_SP11A_VH3_VLK1_Y_ | GFTFSK | 480 | YYDS | 140 | ASYVWVDLDFDH | 553 |
| PTM | QG | | SK | | | |
| CD3_SP11A_VH3_VLK1_S_ | GFTFSK | 480 | YYDS | 140 | ASSSVVDLDFDH | 542 |
| PTM_SW | QG | | SK | | | |
| CD3_SP11A_VH3_VLK1PTM_ | GFTFSK | 480 | YYDS | 140 | ASFSVVDLDFDH | 465 |
| SW | QG | | SK | | | |
| CD3_SP11A_VH3_VLK1_S | GFTFSK | 142 | YYDS | 140 | ASFSVVDLDFDH | 465 |
| W | NG | | SK | | | |
| CD3_SP11A_VH5_VK2_Y | GFTFSK | 480 | YYDS | 140 | ASYVWVDLDFDH | 553 |
| | QG | | SK | | | |
| CD3_SP11A_VH5_VK2_S | GFTFSK | 480 | YYDS | 140 | ASSVWVDLDFDH | 546 |
| | QG | | SK | | | |
| CD3_SP11A_VH5_VK2_Y_ | GFTFSK | 142 | YYDS | 140 | ASYVWVDLDFDH | 553 |
| PTM | NG | | SK | | | |
| CD3_SP11A_VH5_VK2_S_ | GFTFSK | 142 | YYDS | 140 | ASSVWVDLDFDH | 546 |
| PTM | NG | | SK | | | |
| CD3_SP11A_VH5_VK2_Y_ | GFTFSK | 480 | YYDS | 140 | ASYSVVDLDFDH | 549 |
| SW | QG | | SK | | | |
| CD3_SP11A_VH5_VK2_S_ | GFTFSK | 480 | YYDS | 140 | ASSSVVDLDFDH | 542 |
| SW | QG | | SK | | | |
| CD3_SP11A_VH5_VK2_Y_ | GFTFSK | 142 | YYDS | 140 | ASYSVVDLDFDH | 549 |
| PTM_SW | NG | | SK | | | |
| CD3_SP11A_VH5_VK2_S_ | GFTFSK | 142 | YYDS | 140 | ASSSVVDLDFDH | 542 |
| PTM_SW | NG | | SK | | | |
| CD3_SP11A_VH5_VK2_PT | GFTFSK | 142 | YYDS | 140 | ASFSVVDLDFDH | 465 |
| M_SW | NG | | SK | | | |
| CD3_SP11A_VH5_VK2_S | GFTFSK | 480 | YYDS | 140 | ASFSVVDLDFDH | 465 |
| W | QG | | SK | | | |

TABLE 1I-2
CD3 Binders - Light Chain CDR sequences according to combination of Chothia
and IMGT numbering schemes

| | | SEQ ID | | SEQ ID | | SEQ ID |
|---|---|---|---|---|---|---|
| Binder | CDR-L1 | NO: | CDR-L2 | NO: | CDR-L3 | NO: |
| NOV292 | SQSLVRSD | 155 | RVS | 156 | LQSSHF | 151 |
| | GTTY | | | | PWT | |
| NOV123 | SQSLIYSIG | 187 | RVS | 156 | FQSTHL | 183 |
| | NTY | | | | PYT | |
| Sp10b | SQSLIYSIG | 187 | RVS | 156 | FQSTHL | 183 |
| | NTY | | | | PYT | |
| NOV453 | SQNINNY | 219 | NTDHLQA | 223 | LQHRSR | 215 |
| | | | GVP | | YT | |
| NOV229 | SQNINNY | 219 | NTDHLQA | 223 | LQHRSR | 215 |
| | | | GVP | | YT | |
| NOV110 | SQSLVYSH | 283 | RVS | 156 | FQSTHL | 183 |
| | GNTY | | | | PYT | |
| NOV832 | SQSLVYSH | 283 | RVS | 156 | FQSTHL | 183 |
| | GNTY | | | | PYT | |
| NOV589 | SQSLVRSD | 155 | RVS | 156 | LQSSHF | 151 |
| | GTTY | | | | PWT | |
| NOV580 | SQNIDKY | 379 | NTNNLEA | 383 | LQHRSS | 375 |
| | | | GVP | | YT | |
| NOV567 | SQSIGNS | 411 | STSTLEY | 415 | LQYATY | 407 |
| | | | GVP | | PYT | |
| NOV221 | SQNIDKY | 379 | NTNNLEA | 383 | LQHRSG | 439 |
| | | | GVP | | YT | |
| CD3_sp11a_bkm1 | SQSLVRSD | 155 | RVS | 156 | LQSSHF | 151 |
| | GTTY | | | | PWT | |
| CD3_SP11a_bkm2 | SQSLVRSD | 155 | RVS | 156 | LQSSHF | 151 |
| | GTTY | | | | PWT | |
| CD3_sp11a_hz0 | SQSLVRSD | 155 | RVS | 156 | LQSSHF | 151 |
| | GTTY | | | | PWT | |
| CD3_SP11A_HZ1 | SQSLVRSD | 155 | RVS | 156 | LQSSHF | 485 |
| | GTTY | | | | PW | |
| CD3_sp11a_sansPTM_hz1 | SQSLVRSE | 488 | RVS | 156 | LQSSHF | 151 |
| | GTTY | | | | PWT | |
| CD3_sp11a_sansPTM_rat | SQSLVRSE | 488 | RVS | 156 | LQSSHF | 151 |
| | GTTY | | | | PWT | |
| CD3_sp11a_VHVL_YY | SQSLVRSD | 155 | RVS | 156 | LQSSHF | 151 |
| | GTTY | | | | PWT | |
| CD3_SP11A_VHVL_SS | SQSLVRSD | 155 | RVS | 156 | LQSSHF | 151 |
| | GTTY | | | | PWT | |
| CD3_SP11A_VHVL_WS | SQSLVRSD | 155 | RVS | 156 | LQSSHF | 151 |
| | GTTY | | | | PWT | |
| CD3_sp11a_VHVL_SW | SQSLVRSD | 155 | RVS | 156 | LQSSHF | 151 |
| | GTTY | | | | PWT | |
| CD3_SP11A_VHVL_TT | SQSLVRSD | 155 | RVS | 156 | LQSSHF | 151 |
| | GTTY | | | | PWT | |
| CD3_SP11A_VHVL_TW | SQSLVRSD | 155 | RVS | 156 | LQSSHF | 151 |
| | GTTY | | | | PWT | |
| CD3_SP11A_VHVL_VVT | SQSLVRSD | 155 | RVS | 156 | LQSSHF | 151 |
| | GTTY | | | | PWT | |
| CD3_SP11A VH3_VLK_3 | SQSLVRSE | 488 | RVS | 156 | LQSSHF | 151 |
| | GTTY | | | | MATT | |
| CD3_sp11a_VH1_VK2 | SQSLVRSD | 155 | RVS | 156 | LQSSHF | 151 |
| | GTTY | | | | PWT | |
| CD3_SP11A_VH3_VLK1 | SQSLVRSE | 488 | RVS | 156 | LQSSHF | 151 |
| | GTTY | | | | PWT | |
| CD3_SP11A_VH5_VK2 | SQSLVRSD | 155 | RVS | 156 | LQSSHF | 151 |
| | GTTY | | | | PWT | |
| CD3_sp9aFW1_VL_VH_S5 | SQNINNY | 219 | NTDHLQA | 223 | LQHRSR | 215 |
| 6G | | | GVP | | YT | |
| CD3_SP9AFW4_VL_VH_S | SQNINNY | 219 | NTDHLQA | 223 | LQHRSR | 215 |
| 56G | | | GVP | | YT | |
| CD3_sp9aFW1_VLVH | SQNINNY | 219 | NTDHLQA | 223 | LQHRSR | 215 |
| | | | GVP | | YT | |
| CD3_sp9aFW4_VLVH | SQNINNY | 219 | NTDHLQA | 223 | LQHRSR | 215 |
| | | | GVP | | YT | |
| CD3_sp9arabtor_VHVL | SQNINNY | 219 | NTDHLQA | 223 | LQHRSR | 215 |
| | | | GVP | | YT | |
| CD3_sp9arabtor_VLVH | SQNINNY | 219 | NTDHLQA | 223 | LQHRSR | 215 |
| | | | GVP | | YT | |
| CD3_SP11AVH3_VLK_3_Y | SQSLVRSE | 488 | RVS | 156 | LQSSHF | 151 |
| | GTTY | | | | PWT | |
| CD3_SP11AVH3_VLK_3_S | SQSLVRSE | 488 | RVS | 156 | LQSSHF | 151 |
| | GTTY | | | | PWT | |
| CD3_SP11AVH3_VLK_3_Y_ | SQSLVRSD | 155 | RVS | 156 | LQSSHF | 151 |
| PTM | GTTY | | | | PWT | |
| CD3_SP11AVH3_VLK_3_S_ | SQSLVRSD | 155 | RVS | 156 | LQSSHF | 151 |
| PTM | GTTY | | | | PWT | |
| CD3_SP11AVH3_VLK_3_Y_ | SQSLVRSE | 488 | RVS | 156 | LQSSHF | 151 |
| SW | GTTY | | | | PWT | |
| CD3_SP11AVH3_VLK_3_S_ | SQSLVRSE | 488 | RVS | 156 | LQSSHF | 151 |
| SW | GTTY | | | | PWT | |
| CD3_SP11AVH3_VLK_3_Y_ | SQSLVRSD | 155 | RVS | 156 | LQSSHF | 151 |
| PTM_SW | GTTY | | | | PWT | |
| CD3_SP11AVH3_VLK_3_S_ | SQSLVRSD | 155 | RVS | 156 | LQSSHF | 151 |
| SVVPTM | GTTY | | | | PWT | |
| CD3_SP11AVH3_VLK_SW | SQSLVRSD | 155 | RVS | 156 | LQSSHF | 151 |
| PTM | GTTY | | | | PWT | |
| CD3_SP11AVH3_VLK_3_S | SQSLVRSE | 488 | RVS | 156 | LQSSHF | 151 |
| W | GTTY | | | | PWT | |
| CD3_sp11a_VH1_VK2_Y | SQSLVRSD | 155 | RVS | 156 | LQSSHF | 151 |
| | GTTY | | | | PWT | |
| CD3_sp11a_VH1_VK2_S | SQSLVRSD | 155 | RVS | 156 | LQSSHF | 151 |
| | GTTY | | | | PWT | |
| CD3_sp11a_VH1_VK2_Y_ | SQSLVRSE | 488 | RVS | 156 | LQSSHF | 151 |
| PTM | GTTY | | | | PWT | |
| CD3_sp11a_VH1_VK2_S_ | SQSLVRSE | 488 | RVS | 156 | LQSSHF | 151 |
| PTM | GTTY | | | | PWT | |
| CD3_sp11a_VH1_VK2_Y_ | SQSLVRSD | 155 | RVS | 156 | LQSSHF | 151 |
| SW | GTTY | | | | PWT | |
| CD3_sp11a_VH1_VK2_S_ | SQSLVRSD | 155 | RVS | 156 | LQSSHF | 151 |
| SW | GTTY | | | | PWT | |
| CD3_sp11a_VH1_VK2_Y_ | SQSLVRSE | 488 | RVS | 156 | LQSSHF | 151 |
| PTM | GTTY | | | | PWT | |
| CD3_sp11a_VH1_VK2_S_ | SQSLVRSE | 488 | RVS | 156 | LQSSHF | 151 |
| PTM_SW | GTTY | | | | PWT | |
| CD3_sp11a_VH1_VK2_SW | SQSLVRSD | 155 | RVS | 156 | LQSSHF | 151 |
| | GTTY | | | | PWT | |
| CD3_sp11a_VH1_VK2_SW_ | SQSLVRSE | 488 | RVS | 156 | LQSSHF | 151 |
| PTM | GTTY | | | | PWT | |
| CD3_SP11A_VH3_VLK1_Y | SQSLVRSE | 488 | RVS | 156 | LQSSHF | 151 |
| | GTTY | | | | PWT | |
| CD3_SP11A_VH3_VLK1_S | SQSLVRSE | 488 | RVS | 156 | LQSSHF | 151 |
| | GTTY | | | | PWT | |
| CD3_SP11A_VH3_VLK1_Y_ | SQSLVRSD | 155 | RVS | 156 | LQSSHF | 151 |
| PTM | GTTY | | | | PWT | |
| CD3_SP11A_VH3_VLK1_S_ | SQSLVRSD | 155 | RVS | 156 | LQSSHF | 151 |
| PTM | GTTY | | | | PWT | |
| CD3_SP11A_VH3_VLK1_Y_ | SQSLVRSE | 488 | RVS | 156 | LQSSHF | 151 |
| SW | GTTY | | | | PWT | |
| CD3_SP11A_VH3_VLK1_S_ | SQSLVRSE | 488 | RVS | 156 | LQSSHF | 151 |
| SW | GTTY | | | | PWT | |
| CD3_SP11A_VH3_VLK1_Y_ | SQSLVRSD | 155 | RVS | 156 | LQSSHF | 151 |
| PTM | GTTY | | | | PWT | |
| CD3_SP11A_VH3_VLK1_S_ | SQSLVRSD | 155 | RVS | 156 | LQSSHF | 151 |
| PTM_SW | GTTY | | | | PWT | |
| CD3_SP11A_VH3_VLK1PTM_ | SQSLVRSD | 155 | RVS | 156 | LQSSHF | 151 |
| SW | GTTY | | | | PWT | |
| CD3_SP11A_VH3_VLK1_S | SQSLVRSE | 488 | RVS | 156 | LQSSHF | 151 |
| W | GTTY | | | | PWT | |
| CD3_SP11A_VH5_VK2_Y | SQSLVRSD | 155 | RVS | 156 | LQSSHF | 151 |
| | GTTY | | | | PWT | |
| CD3_SP11A_VH5_VK2_S | SQSLVRSD | 155 | RVS | 156 | LQSSHF | 151 |
| | GTTY | | | | PWT | |
| CD3_SP11A_VH5_VK2_Y_ | SQSLVRSD | 155 | RVS | 156 | LQSSHF | 151 |
| PTM | GTTY | | | | PWT | |
| CD3_SP11A_VH5_VK2_S_ | SQSLVRSD | 155 | RVS | 156 | LQSSHF | 151 |
| PTM | GTTY | | | | PWT | |
| CD3_SP11A_VH5_VK2_Y_ | SQSLVRSD | 155 | RVS | 156 | LQSSHF | 151 |
| SW | GTTY | | | | PWT | |
| CD3_SP11A_VH5_VK2_S_ | SQSLVRSD | 155 | RVS | 156 | LQSSHF | 151 |
| SW | GTTY | | | | PWT | |
| CD3_5P11A_VH5_VK2_Y_ | SQSLVRSD | 155 | RVS | 156 | LQSSHF | 151 |
| PTM_SW | GTTY | | | | PWT | |
| CD3_SP11A_VH5_VK2_S_ | SQSLVRSD | 155 | RVS | 156 | LQSSHF | 151 |
| PTM_SW | GTTY | | | | PWT | |
| CD3_SP11A_VH5_VK2_PT | SQSLVRSD | 155 | RVS | 156 | LQSSHF | 151 |
| M_SW | GTTY | | | | PWT | |
| CD3_SP11A_VH5_VK2_S | SQSLVRSD | 155 | RVS | 156 | LQSSHF | 151 |
| W | GTTY | | | | PWT | |

TABLE 1J-1
CD3 Binders-Heavy chain variable sequences

| | | SEQ ID |
|---|---|---|
| Binder | Sequence | NO: |
| NOV292 | QVQLVESGGGVVQPGRSLRLSCAASGFTFSKNGMHVVVRQA | 145 |
| | PGKGLEVVVAMIYYDSSKMYYADTVKGRFTISRDNSKNTLYLQ | |
| | MNSLRAEDTAVYYCASFVWVDLDFDHWGQGTMVTVSS | |
| NOV123 | QVQLVQSGAEVKKPGASVKVSCKASGYTFTSYYIYWVRQAPG | 177 |
| | QRLEVVMGYIYPGHDAIYYSENFKGRVTITADTSASTAYMELSS | |
| | LRSEDTAVYYCVRPNTMMAPLAYWGQGTLVTVSS | |
| Sp10b | QVQLHQSGAELAKPGTSVNLSCKASGYTFTSYYIYVVIKRRPG | 502 |
| | QGLEWIGYIYPGHDAIYYSENFKGKATFTADTSSSTAYMLLGS | |
| | LTPEDSAYYFCVRPNTMMAPLAYWGQGTLVTVSS | |
| NOV453 | QVQLQESGPGLVKPSETLSLTCTVSGFSLTTYNVHWIRQPPG | 209 |
| | KGLEWIGRMRYSGDTSFNAALTSRVTISRDTSKNQVSLKLSSV | |
| | TAADTAVYYCTSDPMYIPNYSYGVMNAWGQGTTVTVSS | |
| NOV229 | QVQLQESGPGLVKPSETLSLTCTVSGFSLTTYNVHWIRQPPG | 241 |
| | KGLEWIGRMRYSGDTSFNAALTSRVTISVDTSKNQFSLKLSSV | |
| | TAADTAVYYCARDPMYIPNYSYGVMNAWGQGTTVTVSS | |
| NOV110 | QVQLVQSGAEVKKPGASVKVSCKASGYTFTSYYIYWVRQAPG | 273 |
| | QRLEVVMGYIYPANGGIYYSEKFKGRVTITADTSAGTAYMELSS | |
| | LRSEDTAVYYCARPVTMMAPLVFWGQGTLVTVSS | |
| NOV832 | QVQLVQSGAEVKKPGASVKVSCKASGYTFTSYYIYWVRQAPG | 305 |
| | QRLEVVMGYIYPANGGIYYSEKFKGRVTITRDTSASTAYMELSS | |
| | LRSEDTAVYYCARPVTMMAPLVFWGQGTLVTVSS | |
| NOV589 | QVQLVESGGGVVQPGRSLRLSCAASGFTFSKNGMHVVVRQA | 337 |
| | PGKGLEVVVAMIYYDSSRMYYADTVKGRFTISRDNSKNTLYLQ | |
| | MNSLRAEDTAVYYCASFVWVDLDFDYWGQGTMVTVSS | |
| NOV580 | QVQLQESGPGLVKPSETLSLTCTVSGFSLTTYNIHWIRQPPGK | 369 |
| | GLEWIGRMRYSGDTSYSSALKSRVTISRDTSKNQVSLKLSSVT | |
| | AADTAVYYCTRDPMYIPGYSYGVMNAWGQGTTVTVSS | |
| NOV567 | QVQLVESGGGVVQPGRSLRLSCAASGFAFRKYGMSVVVRQA | 401 |
| | PGKGLEVVVALIYYDSSKMNYADTVKGRFTISRDNSKNTLYLQ | |
| | MNSLRAEDTAVYYCAALNSEYDWGQGTMVTVSS | |
| NOV221 | QVQLQESGPGLVKPSETLSLTCTVSGFSLTTYNIHWIRQPPGK | 433 |
| | GLEWIGRMRYSGDTSYSSALKSRVTISRDTSKNQVSLKLSSVT | |
| | AADTAVYYCTRDPMYIPGYSYGVMNAWGQGTTVTVSS | |
| CD3_sp11a_bkm1 | QVQLVESGGGVVQPGRSLRLSCAASGFTFSKNGMHVVVRQA | 145 |
| | PGKGLEVVVAMIYYDSSKMYYADTVKGRFTISRDNSKNTLYLQ | |
| | MNSLRAEDTAVYYCASFVWVDLDFDHWGQGTMVTVSS | |
| CD3_SP11a_bkm2 | QVQLVESGGGVVQPGRSLRLSCAASGFTFSKNGMHVVVRQA | 503 |
| | PGKGLEVVVAMIYYDSSKMYYADTVKGRFTISRDNSKNTLYLQ | |
| | MNSLRAEDTAVYYCAKFVWVDLDFDHWGQGTMVTVSS | |
| CD3_sp11a_hz0 | QVQLVESGGGVVQPGRSLRLSCAASGFTFSKNGMHVVVRQA | 503 |
| | PGKGLEVVVAMIYYDSSKMYYADTVKGRFTISRDNSKNTLYLQ | |
| | MNSLRAEDTAVYYCAKFVWVDLDFDHWGQGTMVTVSS | |
| CD3_SP11A_HZ1 | QVQLVESGGGVVQPGRSLRLSCAASGFTFSKNGMHVVVRQA | 145 |
| | PGKGLEVVVAMIYYDSSKMYYADTVKGRFTISRDNSKNTLYLQ | |
| | MNSLRAEDTAVYYCASFVWVDLDFDHWGQGTMVTVSS | |
| CD3_sp11a_sansPTM_hz1 | QVQLVESGGGVVQPGRSLRLSCAASGFTFSKQGMHVVVRQA | 511 |
| | PGKGLEVVVAMIYYDSSKMYYADTVKGRFTISRDNSKNTLYLQ | |
| | MNSLRAEDTAVYYCASFVWVDLDFDHWGQGTMVTVSS | |
| CD3_sp11a_sansPTM_rat | EVKLVESGGDLVQPGDSLTLSCVASGFTFSKQGMHWIRQAPK | 496 |
| | KGLEWIAMIYYDSSKMYYADTVKGRFTISRDNSKNTLYLEMNS | |
| | LRSEDTAMYYCASFVWVDLDFDHWGQGVMVTVSS | |
| CD3_sp11a_VHVL_YY | QVQLVESGGGVVQPGRSLRLSCAASGFTFSKNGMHVVVRQA | 510 |
| | PGKGLEVVVAMIYYDSSKMYYADTVKGRFTISRDNSKNTLYLQ | |
| | MNSLRAEDTAVYYCASFYYDLDFDHWGQGTMVTVSS | |
| CD3_SP11A_VHVL_SS | QVQLVESGGGVVQPGRSLRLSCAASGFTFSKNGMHVVVRQA | 504 |
| | PGKGLEVVVAMIYYDSSKMYYADTVKGRFTISRDNSKNTLYLQ | |
| | MNSLRAEDTAVYYCASFSSDLDFDHWGQGTMVTVSS | |
| CD3_SP11A_VHVL_WS | QVQLVESGGGVVQPGRSLRLSCAASGFTFSKNGMHVVVRQA | 508 |
| | PGKGLEVVVAMIYYDSSKMYYADTVKGRFTISRDNSKNTLYLQ | |
| | MNSLRAEDTAVYYCASFWSDLDFDHWGQGTMVTVSS | |
| CD3_sp11a_VHVL_SW | QVQLVESGGGVVQPGRSLRLSCAASGFTFSKNGMHVVVRQA | 505 |
| | PGKGLEVVVAMIYYDSSKMYYADTVKGRFTISRDNSKNTLYLQ | |
| | MNSLRAEDTAVYYCASFSWDLDFDHWGQGTMVTVSS | |
| CD3_SP11A_VHVL_TT | QVQLVESGGGVVQPGRSLRLSCAASGFTFSKNGMHVVVRQA | 506 |
| | PGKGLEVVVAMIYYDSSKMYYADTVKGRFTISRDNSKNTLYLQ | |
| | MNSLRAEDTAVYYCASFTTDLDFDHWGQGTMVTVSS | |
| CD3_SP11A_VHVL_TW | QVQLVESGGGVVQPGRSLRLSCAASGFTFSKNGMHVVVRQA | 507 |
| | PGKGLEVVVAMIYYDSSKMYYADTVKGRFTISRDNSKNTLYLQ | |
| | MNSLRAEDTAVYYCASFTVVDLDFDHWGQGTMVTVSS | |
| CD3_SP11A_VHVL_VVT | QVQLVESGGGVVQPGRSLRLSCAASGFTFSKNGMHVVVRQA | 509 |
| | PGKGLEVVVAMIYYDSSKMYYADTVKGRFTISRDNSKNTLYLQ | |
| | MNSLRAEDTAVYYCASFVVTDLDFDHWGQGTMVTVSS | |
| CD3_SP11A_VH3_VLK_3 | QVQLVESGGGVVQPGRSLRLSCAASGFTFSKNGMHVVVRQA | 145 |
| | PGKGLEVVVAMIYYDSSKMYYADTVKGRFTISRDNSKNTLYLQ | |
| | MNSLRAEDTAVYYCASFVWVDLDFDHWGQGTMVTVSS | |
| CD3_sp11a_VH1_VK2 | QVQLVQSGAEVKKPGASVKVSCKASGFTFSKQGMHVVVRQA | 512 |
| | PGQGLEWMGMIYYDSSKMYYADTVKGRVTMTRDTSTNTLYM | |
| | ELSSLRSEDTAVYYCASFVWVDLDFDHWGQGTMVTVSS | |
| CD3_SP11A_VH3_VLK1 | QVQLVESGGGVVQPGRSLRLSCAASGFTFSKNGMHVVVRQA | 145 |
| | PGKGLEVVVAMIYYDSSKMYYADTVKGRFTISRDNSKNTLYLQ | |
| | MNSLRAEDTAVYYCASFVWVDLDFDHWGQGTMVTVSS | |
| CD3_SP11A_VH5_VK2 | EVQLVQSGAEVKKPGESLKISCKGSGFTFSKQGMHVVVRQMP | 501 |
| | GKGLEWMGMIYYDSSKMYYADTVKGQVTISRDNSINTLYLQW | |
| | SSLKASDTAMYYCASFVWVDLDFDHWGQGTMVTVSS | |
| CD3_sp9aFW1_VL_VH_ | EVQLVESGGGLVQPGGSLRLSCAASGFSLTTYNVHVVVRQAP | 497 |
| S56G | GKGLEVVVGRMRYSGDTSFNAALTSRFTISRDNSKNTLYLQMN | |
| | SLRAEDTAVYYCASDPMYIPNYAYGVMNAWGQGTLVTVSS | |
| CD3_SP9AFW4_VL_VH_ | EVQLVETGGGLVQPGGSRRLSCAASGFSLTTYNVHVVVRQAP | 499 |
| S56G | GKGLEVVVGRMRYSGDTSFNAALTSRFTISRDTSKNTVYLQMN | |
| | SLRAEDTGVYYCASDPMYIPNYAYGVMNAWGQGTLVTVSS | |
| CD3_sp9aFW1_VLVH | EVQLVETGGGLVQPGGSRRLSCAASGFSLTTYNVHVVVRQAP | 500 |
| | GKGLEVVVSRMRYSGDTSFNAALTSRFTISRDTSKNTVYLQMN | |
| | SLRAEDTGVYYCASDPMYIPNYAYGVMNAWGQGTLVTVSS | |
| CD3_sp9aFW4_VLVH | VQLVESGGGLVQPGGSLRLSCAASGFSLTTYNVHVVVRQAPG | 513 |
| | KGLEVVVSRMRYSGDTSFNAALTSRFTISRDNSKNTLYLQMNS | |
| | LRAEDTAVYYCASDPMYIPNYAYGVMNAWGQGTLVTVSS | |
| CD3_sp9arabtor_VHVL | EVQLVESGGGSVQPGGSLRLSCTASGFSLTTYNVHVVVRQAP | 498 |
| | GKGLEVVVGRMRYSGDTSFNAALTSRFTISRDTSKNTVYLQMN | |
| | SLRAEDTATYYCASDPMYIPNYAYGVMNAWGQGTTVTVSS | |
| CD3_sp9arabtor_VLVH | EVQLVESGGGSVQPGGSLRLSCTASGFSLTTYNVHVVVRQAP | 498 |
| | GKGLEVVVGRMRYSGDTSFNAALTSRFTISRDTSKNTVYLQMN | |
| | SLRAEDTATYYCASDPMYIPNYAYGVMNAWGQGTTVTVSS | |
| CD3_sp11a_VHVL_YY_ | QVQLVESGGGVVQPGRSLRLSCAASGFTFSKQGMHVVVRQA | 588 |
| SANSPTM | PGKGLEVVVAMIYYDSSKMYYADTVKGRFTISRDNSKNTLYLQ | |
| | MNSLRAEDTAVYYCASFYYDLDFDHWGQGTMVTVSS | |
| CD3_sp11a_VHVL_YY_ | QVQLVESGGGVVQPGRSLRLSCAASGFTFSKQGMHVVVRQA | 602 |
| SANSPTM_Y | PGKGLEVVVAMIYYDSSKMYYADTVKGRFTISRDNSKNTLYLQ | |
| | MNSLRAEDTAVYYCASYYYDLDFDHWGQGTMVTVSS | |
| CD3_sp11a_VHVL_YY_ | QVQLVESGGGVVQPGRSLRLSCAASGFTFSKQGMHVVVRQA | 595 |
| SANPTM_S | PGKGLEVVVAMIYYDSSKMYYADTVKGRFTISRDNSKNTLYLQ | |
| | MNSLRAEDTAVYYCASSYYDLDFDHWGQGTMVTVSS | |
| CD3_sp11a_VHVL_YY_Y | QVQLVESGGGVVQPGRSLRLSCAASGFTFSKNGMHVVVRQA | 571 |
| | PGKGLEVVVAMIYYDSSKMYYADTVKGRFTISRDNSKNTLYLQ | |
| | MNSLRAEDTAVYYCASYYYDLDFDHWGQGTMVTVSS | |
| CD3_sp11a_VHVL_YY_s | QVQLVESGGGVVQPGRSLRLSCAASGFTFSKNGMHVVVRQA | 577 |
| | PGKGLEVVVAMIYYDSSKMYYADTVKGRFTISRDNSKNTLYLQ | |
| | MNSLRAEDTAVYYCASSYYDLDFDHWGQGTMVTVSS | |
| CD3_sp11a_VHVL_SS_ | QVQLVESGGGVVQPGRSLRLSCAASGFTFSKQGMHVVVRQA | 583 |
| SANSPTM | PGKGLEVVVAMIYYDSSKMYYADTVKGRFTISRDNSKNTLYLQ | |
| | MNSLRAEDTAVYYCASFSSDLDFDHWGQGTMVTVSS | |
| CD3_sp11a_VHVL_SS_ | QVQLVESGGGVVQPGRSLRLSCAASGFTFSKQGMHVVVRQA | 596 |
| SANSPTM_Y | PGKGLEVVVAMIYYDSSKMYYADTVKGRFTISRDNSKNTLYLQ | |
| | MNSLRAEDTAVYYCASYSSDLDFDHWGQGTMVTVSS | |
| CD3_sp11a_VHVL_SS_ | QVQLVESGGGVVQPGRSLRLSCAASGFTFSKQGMHVVVRQA | 589 |
| SANSPTM_S | PGKGLEVVVAMIYYDSSKMYYADTVKGRFTISRDNSKNTLYLQ | |
| | MNSLRAEDTAVYYCASSSSDLDFDHWGQGTMVTVSS | |
| CD3_sp11a_VHVL_SS_Y | QVQLVESGGGVVQPGRSLRLSCAASGFTFSKNGMHVVVRQA | 570 |
| | PGKGLEVVVAMIYYDSSKMYYADTVKGRFTISRDNSKNTLYLQ | |
| | MNSLRAEDTAVYYCASYSSDLDFDHWGQGTMVTVSS | |
| CD3_sp11a_VHVL_SS_S | QVQLVESGGGVVQPGRSLRLSCAASGFTFSKNGMHVVVRQA | 572 |
| | PGKGLEVVVAMIYYDSSKMYYADTVKGRFTISRDNSKNTLYLQ | |
| | MNSLRAEDTAVYYCASSSSDLDFDHWGQGTMVTVSS | |
| CD3_sp11 a_VHVL_SS_ | QVQLVESGGGVVQPGRSLRLSCAASGFTFSKQGMHVVVRQA | 583 |
| SANSPTM | PGKGLEVVVAMIYYDSSKMYYADTVKGRFTISRDNSKNTLYLQ | |
| | MNSLRAEDTAVYYCASFSSDLDFDHWGQGTMVTVSS | |
| CD3_sp11a_VHVL_WS_ | QVQLVESGGGVVQPGRSLRLSCAASGFTFSKQGMHVVVRQA | 600 |
| SANSPTM _Y | PGKGLEVVVAMIYYDSSKMYYADTVKGRFTISRDNSKNTLYLQ | |
| | MNSLRAEDTAVYYCASYWSDLDFDHWGQGTMVTVSS | |
| CD3_sp11a_VHVL_WS_ | QVQLVESGGGVVQPGRSLRLSCAASGFTFSKQGMHVVVRQA | 593 |
| SANSPTM_S | PGKGLEVVVAMIYYDSSKMYYADTVKGRFTISRDNSKNTLYLQ | |
| | MNSLRAEDTAVYYCASSWSDLDFDHWGQGTMVTVSS | |
| CD3_sp11a_VHVL_WS_Y | QVQLVESGGGVVQPGRSLRLSCAASGFTFSKNGMHVVVRQA | 581 |
| | PGKGLEVVVAMIYYDSSKMYYADTVKGRFTISRDNSKNTLYLQ | |
| | MNSLRAEDTAVYYCASYWSDLDFDHWGQGTMVTVSS | |
| CD3_sp11a_VHVL_WS_S | QVQLVESGGGVVQPGRSLRLSCAASGFTFSKNGMHVVVRQA | 569 |
| | PGKGLEVVVAMIYYDSSKMYYADTVKGRFTISRDNSKNTLYLQ | |
| | MNSLRAEDTAVYYCASSWSDLDFDHWGQGTMVTVSS | |
| CD3_sp11a_VHVL_WS_ | QVQLVESGGGVVQPGRSLRLSCAASGFTFSKQGMHVVVRQA | 587 |
| SANSPTM | PGKGLEVVVAMIYYDSSKMYYADTVKGRFTISRDNSKNTLYLQ | |
| | MNSLRAEDTAVYYCASFWSDLDFDHWGQGTMVTVSS | |
| CD3_sp11a_VHVL_SW_ | QVQLVESGGGVVQPGRSLRLSCAASGFTFSKQGMHVVVRQA | 597 |
| SANSPTM_Y | PGKGLEVVVAMIYYDSSKMYYADTVKGRFTISRDNSKNTLYLQ | |
| | MNSLRAEDTAVYYCASYSWDLDFDHWGQGTMVTVSS | |
| CD3_sp11a_VHVL_SW_ | QVQLVESGGGVVQPGRSLRLSCAASGFTFSKQGMHVVVRQA | 590 |
| SANSPTM_S | PGKGLEVVVAMIYYDSSKMYYADTVKGRFTISRDNSKNTLYLQ | |
| | MNSLRAEDTAVYYCASSSWDLDFDHWGQGTMVTVSS | |
| CD3_sp11a_VHVL_SW_Y | QVQLVESGGGVVQPGRSLRLSCAASGFTFSKNGMHVVVRQA | 578 |
| | PGKGLEVVVAMIYYDSSKMYYADTVKGRFTISRDNSKNTLYLQ | |
| | MNSLRAEDTAVYYCASYSWDLDFDHWGQGTMVTVSS | |
| CD3_sp11a_VHVL_SW_S | QVQLVESGGGVVQPGRSLRLSCAASGFTFSKNGMHVVVRQA | 573 |
| | PGKGLEVVVAMIYYDSSKMYYADTVKGRFTISRDNSKNTLYLQ | |
| | MNSLRAEDTAVYYCASSSWDLDFDHWGQGTMVTVSS | |
| CD3_sp11a_VHVL_SW_ | QVQLVESGGGVVQPGRSLRLSCAASGFTFSKQGMHVVVRQA | 584 |
| SANSPTM | PGKGLEVVVAMIYYDSSKMYYADTVKGRFTISRDNSKNTLYLQ | |
| | MNSLRAEDTAVYYCASFSWDLDFDHWGQGTMVTVSS | |
| CD3_sp11a_VHVL_TW_ | QVQLVESGGGVVQPGRSLRLSCAASGFTFSKQGMHVVVRQA | 599 |
| SANSPTM_Y | PGKGLEVVVAMIYYDSSKMYYADTVKGRFTISRDNSKNTLYLQ | |
| | MNSLRAEDTAVYYCASYTWDLDFDHWGQGTMVTVSS | |
| CD3_sp11a_VHVL_TW_ | QVQLVESGGGVVQPGRSLRLSCAASGFTFSKQGMHVVVRQA | 592 |
| SANSPTM _S | PGKGLEVVVAMIYYDSSKMYYADTVKGRFTISRDNSKNTLYLQ | |
| | MNSLRAEDTAVYYCASSTWDLDFDHWGQGTMVTVSS | |
| CD3_sp11a_VHVL_TW_Y | QVQLVESGGGVVQPGRSLRLSCAASGFTFSKNGMHVVVRQA | 580 |
| | PGKGLEVVVAMIYYDSSKMYYADTVKGRFTISRDNSKNTLYLQ | |
| | MNSLRAEDTAVYYCASYTWDLDFDHWGQGTMVTVSS | |
| CD3_sp11a_VHVL_TW_S | QVQLVESGGGVVQPGRSLRLSCAASGFTFSKNGMHVVVRQA | 575 |
| | PGKGLEVVVAMIYYDSSKMYYADTVKGRFTISRDNSKNTLYLQ | |
| | MNSLRAEDTAVYYCASSTWDLDFDHWGQGTMVTVSS | |
| CD3_sp11a_VHVL_T_ | WQVQLVESGGGVVQPGRSLRLSCAASGFTFSKQGMHVVVRQA | 586 |
| SANSPTM | PGKGLEVVVAMIYYDSSKMYYADTVKGRFTISRDNSKNTLYLQ | |
| | MNSLRAEDTAVYYCASFTVVDLDFDHWGQGTMVTVSS | |
| CD3_sp11a_VHVL_TT_ | QVQLVESGGGVVQPGRSLRLSCAASGFTFSKQGMHVVVRQA | 598 |
| SANSPTM_Y | PGKGLEVVVAMIYYDSSKMYYADTVKGRFTISRDNSKNTLYLQ | |
| | MNSLRAEDTAVYYCASYTTDLDFDHWGQGTMVTVSS | |
| CD3_sp11a_VHVL_TT_S | QVQLVESGGGVVQPGRSLRLSCAASGFTFSKQGMHVVVRQA | 591 |
| ANSPTM_S | PGKGLEVVVAMIYYDSSKMYYADTVKGRFTISRDNSKNTLYLQ | |
| | MNSLRAEDTAVYYCASSTTDLDFDHWGQGTMVTVSS | |
| CD3_sp11a_VHVL_TT_Y | QVQLVESGGGVVQPGRSLRLSCAASGFTFSKNGMHVVVRQA | 579 |
| | PGKGLEVVVAMIYYDSSKMYYADTVKGRFTISRDNSKNTLYLQ | |
| | MNSLRAEDTAVYYCASYTTDLDFDHWGQGTMVTVSS | |
| CD3_sp11a_VHVL_TT_S | QVQLVESGGGVVQPGRSLRLSCAASGFTFSKNGMHVVVRQA | 574 |
| | PGKGLEVVVAMIYYDSSKMYYADTVKGRFTISRDNSKNTLYLQ | |
| | MNSLRAEDTAVYYCASSTTDLDFDHWGQGTMVTVSS | |
| CD3_sp11a_VHVL_TT_S | QVQLVESGGGVVQPGRSLRLSCAASGFTFSKQGMHVVVRQA | 585 |
| ANSPTM | PGKGLEVVVAMIYYDSSKMYYADTVKGRFTISRDNSKNTLYLQ | |
| | MNSLRAEDTAVYYCASFTTDLDFDHWGQGTMVTVSS | |
| CD3_SP11AVH3_VLK_3_Y | QVQLVESGGGVVQPGRSLRLSCAASGFTFSKNGMHVVVRQA | 582 |
| | PGKGLEVVVAMIYYDSSKMYYADTVKGRFTISRDNSKNTLYLQ | |
| | MNSLRAEDTAVYYCASYVWVDLDFDHWGQGTMVTVSS | |
| CD3_SP11AVH3_VLK_3_S | QVQLVESGGGVVQPGRSLRLSCAASGFTFSKNGMHVVVRQA | 576 |
| | PGKGLEVVVAMIYYDSSKMYYADTVKGRFTISRDNSKNTLYLQ | |
| | MNSLRAEDTAVYYCASSVWVDLDFDHWGQGTMVTVSS | |
| CD3_SP11AVH3_VLK_3_ | QVQLVESGGGVVQPGRSLRLSCAASGFTFSKNGMHVVVRQA | 582 |
| Y_PTM | PGKGLEVVVAMIYYDSSKMYYADTVKGRFTISRDNSKNTLYLQ | |
| | MNSLRAEDTAVYYCASYVWVDLDFDHWGQGTMVTVSS | |
| CD3_SP11AVH3_VLK_3_ | QVQLVESGGGVVQPGRSLRLSCAASGFTFSKNGMHVVVRQA | 576 |
| S_PTM | PGKGLEVVVAMIYYDSSKMYYADTVKGRFTISRDNSKNTLYLQ | |
| | MNSLRAEDTAVYYCASSVWVDLDFDHWGQGTMVTVSS | |
| CD3_SP11AVH3_VLK_3_ | QVQLVESGGGVVQPGRSLRLSCAASGFTFSKNGMHVVVRQA | 578 |
| Y_SW | PGKGLEVVVAMIYYDSSKMYYADTVKGRFTISRDNSKNTLYLQ | |
| | MNSLRAEDTAVYYCASYSWDLDFDHWGQGTMVTVSS | |
| CD3_SP11AVH3_VLK_3_ | QVQLVESGGGVVQPGRSLRLSCAASGFTFSKNGMHVVVRQA | 573 |
| S_SW | PGKGLEVVVAMIYYDSSKMYYADTVKGRFTISRDNSKNTLYLQ | |
| | MNSLRAEDTAVYYCASSSWDLDFDHWGQGTMVTVSS | |
| CD3_SP11AVH3_VLK_3_ | QVQLVESGGGVVQPGRSLRLSCAASGFTFSKNGMHVVVRQA | 578 |
| Y_PTM_SW | PGKGLEVVVAMIYYDSSKMYYADTVKGRFTISRDNSKNTLYLQ | |
| | MNSLRAEDTAVYYCASYSWDLDFDHWGQGTMVTVSS | |
| CD3_SP11AVH3_VLK_3_ | QVQLVESGGGVVQPGRSLRLSCAASGFTFSKNGMHVVVRQA | 573 |
| S_SVVPTM | PGKGLEVVVAMIYYDSSKMYYADTVKGRFTISRDNSKNTLYLQ | |
| | MNSLRAEDTAVYYCASSSWDLDFDHWGQGTMVTVSS | |
| CD3_SP11AVH3_VLK_S | QVQLVESGGGVVQPGRSLRLSCAASGFTFSKNGMHVVVRQA | 505 |
| VVPTM | PGKGLEVVVAMIYYDSSKMYYADTVKGRFTISRDNSKNTLYLQ | |
| | MNSLRAEDTAVYYCASFSWDLDFDHWGQGTMVTVSS | |
| CD3_SP11AVH3_VLK_3_ | QVQLVESGGGVVQPGRSLRLSCAASGFTFSKNGMHVVVRQA | 505 |
| SW | PGKGLEVVVAMIYYDSSKMYYADTVKGRFTISRDNSKNTLYLQ | |
| | MNSLRAEDTAVYYCASFSWDLDFDHWGQGTMVTVSS | |
| CD3_sp11a_VH1_VK2_Y | QVQLVQSGAEVKKPGASVKVSCKASGFTFSKQGMHVVVRQA | 612 |
| | PGQGLEWMGMIYYDSSKMYYADTVKGRVTMTRDTSTNTLYM | |
| | ELSSLRSEDTAVYYCASYVWVDLDFDHWGQGTMVTVSS | |
| CD3_sp11a_VH1_VK2_S | QVQLVQSGAEVKKPGASVKVSCKASGFTFSKQGMHVVVRQA | 610 |
| | PGQGLEWMGMIYYDSSKMYYADTVKGRVTMTRDTSTNTLYM | |
| | ELSSLRSEDTAVYYCASSVWVDLDFDHWGQGTMVTVSS | |
| CD3_sp11a_VH1_VK2_Y_ | QVQLVQSGAEVKKPGASVKVSCKASGFTFSKNGMHVVVRQAP | 604 |
| PTM | GQGLEVVMGMIYYDSSKMYYADTVKGRVTMTRDTSTNTLYME | |
| | LSSLRSEDTAVYYCASYVWVDLDFDHWGQGTMVTVSS | |
| CD3_sp11a_VH1_VK2_S_ | QVQLVQSGAEVKKPGASVKVSCKASGFTFSKQGMHVVVRQA | 607 |
| PTM | PGNGLEWMGMIYYDSSKMYYADTVKGRVTMTRDTSTNTLYM | |
| | ELSSLRSEDTAVYYCASSVWVDLDFDHWGQGTMVTVSS | |
| CD3_sp11a_VH1_VK2_Y_ | QVQLVQSGAEVKKPGASVKVSCKASGFTFSKQGMHVVVRQA | 611 |
| SW | PGQGLEWMGMIYYDSSKMYYADTVKGRVTMTRDTSTNTLYM | |
| | ELSSLRSEDTAVYYCASYSWDLDFDHWGQGTMVTVSS | |
| CD3_sp11a_VH1_VK2_S_ | QVQLVQSGAEVKKPGASVKVSCKASGFTFSKQGMHVVVRQA | 609 |
| SW | PGQGLEWMGMIYYDSSKMYYADTVKGRVTMTRDTSTNTLYM | |
| | ELSSLRSEDTAVYYCASSSWDLDFDHWGQGTMVTVSS | |
| CD3_sp11a_VH1_VK2_Y_ | QVQLVQSGAEVKKPGASVKVSCKASGFTFSKNGMHVVVRQAP | 603 |
| PTM | GQGLEVVMGMIYYDSSKMYYADTVKGRVTMTRDTSTNTLYME | |
| | LSSLRSEDTAVYYCASYSWDLDFDHWGQGTMVTVSS | |
| CD3_sp11a_VH1_VK2_S_ | QVQLVQSGAEVKKPGASVKVSCKASGFTFSKQGMHVVVRQA | 606 |
| PTM_SW | PGNGLEWMGMIYYDSSKMYYADTVKGRVTMTRDTSTNTLYM | |
| | ELSSLRSEDTAVYYCASSSWDLDFDHWGQGTMVTVSS | |
| CD3_sp11a_VH1_VK2_SW | QVQLVQSGAEVKKPGASVKVSCKASGFTFSKQGMHVVVRQA | 608 |
| | PGQGLEWMGMIYYDSSKMYYADTVKGRVTMTRDTSTNTLYM | |
| | ELSSLRSEDTAVYYCASFSWDLDFDHWGQGTMVTVSS | |
| CD3_sp11a_VH1_VK2_S | QVQLVQSGAEVKKPGASVKVSCKASGFTFSKQGMHVVVRQA | 605 |
| WPTM | PGNGLEWMGMIYYDSSKMYYADTVKGRVTMTRDTSTNTLYM | |
| | ELSSLRSEDTAVYYCASFSWDLDFDHWGQGTMVTVSS | |
| CD3_SP11A_VH3_VLK1_Y | QVQLVESGGGVVQPGRSLRLSCAASGFTFSKNGMHVVVRQA | 582 |
| | PGKGLEVVVAMIYYDSSKMYYADTVKGRFTISRDNSKNTLYLQ | |
| | MNSLRAEDTAVYYCASYVWVDLDFDHWGQGTMVTVSS | |
| CD3_SP11A_VH3_VLK1_S | QVQLVESGGGVVQPGRSLRLSCAASGFTFSKNGMHVVVRQA | 576 |
| | PGKGLEVVVAMIYYDSSKMYYADTVKGRFTISRDNSKNTLYLQ | |
| | MNSLRAEDTAVYYCASSVWVDLDFDHWGQGTMVTVSS | |
| CD3_SP11A_VH3_VLK1_ | QVQLVESGGGVVQPGRSLRLSCAASGFTFSKQGMHVVVRQA | 601 |
| Y_PTM | PGKGLEVVVAMIYYDSSKMYYADTVKGRFTISRDNSKNTLYLQ | |
| | MNSLRAEDTAVYYCASYVWVDLDFDHWGQGTMVTVSS | |
| CD3_SP11A_VH3_VLK1_ | QVQLVESGGGVVQPGRSLRLSCAASGFTFSKQGMHVVVRQA | 594 |
| S_PTM | PGKGLEVVVAMIYYDSSKMYYADTVKGRFTISRDNSKNTLYLQ | |
| | MNSLRAEDTAVYYCASSVWVDLDFDHWGQGTMVTVSS | |
| CD3_SP11A_VH3_VLK1 | QVQLVESGGGVVQPGRSLRLSCAASGFTFSKNGMHVVVRQA | 578 |
| Y_SW | PGKGLEVVVAMIYYDSSKMYYADTVKGRFTISRDNSKNTLYLQ | |
| | MNSLRAEDTAVYYCASYSWDLDFDHWGQGTMVTVSS | |
| CD3_SP11A_VH3_VLK1_ | QVQLVESGGGVVQPGRSLRLSCAASGFTFSKNGMHVVVRQA | 576 |
| S_SW | PGKGLEVVVAMIYYDSSKMYYADTVKGRFTISRDNSKNTLYLQ | |
| | MNSLRAEDTAVYYCASSVWVDLDFDHWGQGTMVTVSS | |
| CD3_SP11A_VH3_VLK1_ | QVQLVESGGGVVQPGRSLRLSCAASGFTFSKQGMHVVVRQA | 601 |
| Y_PTM | PGKGLEVVVAMIYYDSSKMYYADTVKGRFTISRDNSKNTLYLQ | |
| | MNSLRAEDTAVYYCASYVWVDLDFDHWGQGTMVTVSS | |
| CD3_SP11A_VH3_VLK1_ | QVQLVESGGGVVQPGRSLRLSCAASGFTFSKQGMHVVVRQA | 590 |
| S_PTM_SW | PGKGLEVVVAMIYYDSSKMYYADTVKGRFTISRDNSKNTLYLQ | |
| | MNSLRAEDTAVYYCASSSWDLDFDHWGQGTMVTVSS | |
| CD3_SP11A_VH3_VLK1 | QVQLVESGGGVVQPGRSLRLSCAASGFTFSKQGMHVVVRQA | 584 |
| PTM_SW | PGKGLEVVVAMIYYDSSKMYYADTVKGRFTISRDNSKNTLYLQ | |
| | MNSLRAEDTAVYYCASFSWDLDFDHWGQGTMVTVSS | |
| CD3_SP11A_VH3_VLK1_ | QVQLVESGGGVVQPGRSLRLSCAASGFTFSKNGMHVVVRQA | 505 |
| SW | PGKGLEVVVAMIYYDSSKMYYADTVKGRFTISRDNSKNTLYLQ | |
| | MNSLRAEDTAVYYCASFSWDLDFDHWGQGTMVTVSS | |
| CD3_SP11A_VH5_VK2_Y | EVQLVQSGAEVKKPGESLKISCKGSGFTFSKQGMHVVVRQMP | 566 |
| | GKGLEWMGMIYYDSSKMYYADTVKGQVTISRDNSINTLYLQW | |
| | SSLKASDTAMYYCASYVWVDLDFDHWGQGTMVTVSS | |
| CD3_SP11A_VH5_VK2_S | EVQLVQSGAEVKKPGESLKISCKGSGFTFSKQGMHVVVRQMP | 564 |
| | GKGLEWMGMIYYDSSKMYYADTVKGQVTISRDNSINTLYLQW | |
| | SSLKASDTAMYYCASSVWVDLDFDHWGQGTMVTVSS | |
| CD3_SP11A_VH5_VK2_ | EVQLVQSGAEVKKPGESLKISCKGSGFTFSKNGMHVVVRQMP | 561 |
| Y_PTM | GKGLEWMGMIYYDSSKMYYADTVKGQVTISRDNSINTLYLQW | |
| | SSLKASDTAMYYCASYVWVDLDFDHWGQGTMVTVSS | |
| CD3_SP11A_VH5_VK2_ | EVQLVQSGAEVKKPGESLKISCKGSGFTFSKNGMHVVVRQMP | 559 |
| S_PTM | GKGLEWMGMIYYDSSKMYYADTVKGQVTISRDNSINTLYLQW | |
| | SSLKASDTAMYYCASSVWVDLDFDHWGQGTMVTVSS | |
| CD3_SP11A_VH5_VK2_ | EVQLVQSGAEVKKPGESLKISCKGSGFTFSKQGMHVVVRQMP | 565 |
| Y_SW | GKGLEWMGMIYYDSSKMYYADTVKGQVTISRDNSINTLYLQW | |
| | SSLKASDTAMYYCASYSVVDLDFDHWGQGTMVTVSS | |
| CD3_SP11A_VH5_VK2_ | EVQLVQSGAEVKKPGESLKISCKGSGFTFSKQGMHVVVRQMP | 563 |
| S_SW | GKGLEWMGMIYYDSSKMYYADTVKGQVTISRDNSINTLYLQW | |
| | SSLKASDTAMYYCASSSVVDLDFDHWGQGTMVTVSS | |
| CD3_SP11A_VH5_VK2_ | EVQLVQSGAEVKKPGESLKISCKGSGFTFSKNGMHVVVRQMP | 560 |
| Y_PTM_SW | GKGLEWMGMIYYDSSKMYYADTVKGQVTISRDNSINTLYLQW | |
| | SSLKASDTAMYYCASYSVVDLDFDHWGQGTMVTVSS | |
| CD3_SP11A_VH5_VK2_ | EVQLVQSGAEVKKPGESLKISCKGSGFTFSKNGMHVVVRQMP | 558 |
| S_PTM_SW | GKGLEWMGMIYYDSSKMYYADTVKGQVTISRDNSINTLYLQW | |
| | SSLKASDTAMYYCASSSVVDLDFDHWGQGTMVTVSS | |
| CD3_SP11A_VH5_VK2_ | EVQLVQSGAEVKKPGESLKISCKGSGFTFSKNGMHVVVRQMP | 557 |
| PTM_SW | GKGLEWMGMIYYDSSKMYYADTVKGQVTISRDNSINTLYLQW | |
| | SSLKASDTAMYYCASFSVVDLDFDHWGQGTMVTVSS | |
| CD3_SP11A_VH5_VK2_ | EVQLVQSGAEVKKPGESLKISCKGSGFTFSKQGMHVVVRQMP | 562 |
| SW | GKGLEWMGMIYYDSSKMYYADTVKGQVTISRDNSINTLYLQW | |
| | SSLKASDTAMYYCASFSVVDLDFDHWGQGTMVTVSS | |

TABLE 1J-2
CD3 Binders - Light chain variable sequences

| | | SEQ ID |
|---|---|---|
| Binder | Sequence | NO: |
| NOV292 | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| NOV123 | DVVMTQSPLSLPVTLGQPASISCRSSQSLIYSIGNTYLHVVYQQ | 193 |
| | RPGQSPRLLIYRVSNRFSGVPDRFSGSGSGTDFTLKISRVEAE | |
| | DVGVYYCFQSTHLPYTFGQGTKLEIK | |
| Sp10b | VVVLTQTPVSLPVSLGGQASISCRSSQSLIYSIGNTYLHVVYLQ | 514 |
| | KPGQSPQLLIYRVSNRFSGVPDRFSGSGSGTDFTLKISRVEPE | |
| | DLGDYYCFQSTHLPYTFGAGTKLELK | |
| NOV453 | DIQMTQSPSSLSASVGDRVTITCKASQNINNYLNVVYQQKPGK | 225 |
| | APKLLIYNTDHLQAGVPSRFSGSGSGTDYTLTISSLQPEDFATY | |
| | FCLQHRSRYTFGPGTKVDIK | |
| NOV229 | DIQMTQSPSSLSASVGDRVTITCKASQNINNYLNVVYQQKPGK | 257 |
| | APKLLIYNTDHLQAGVPSRFSGSGSGTDFTLTISSLQPEDFATY | |
| | YCLQHRSRYTFGPGTKVDIK | |
| NOV110 | DVVMTQSPLSLPVTLGQPASISCRSSQSLVYSHGNTYLHVVYQ | 289 |
| | QRPGQSPRLLIYRVSNRFSGVPDRFSGSGSGTDFTLKISRVEA | |
| | EDVGVYYCFQSTHLPYTFGQGTKLEIK | |
| NOV832 | DVVMTQSPLSLPVTLGQPASISCRSSQSLVYSHGNTYLHVVFQ | 321 |
| | QRPGQSPRRLIYRVSNRFSGVPDRFSGSGSGTDFTLKISRVE | |
| | AEDVGVYYCFQSTHLPYTFGQGTKLEIK | |
| NOV589 | DIVMTQTPLSSPVTLGQPASISCRSSQSLVRSDGTTYFNVVYQ | 353 |
| | QRPGQPPRLLIYRVSNRFSGVPDRFSGSGAGTDFTLKISRVEA | |
| | EDVGVYYCLQSSHFPVVTFGGGTKVEIK | |
| NOV580 | DIQMTQSPSSLSASVGDRVTITCKTSQNIDKYLNVVYQQKPGK | 385 |
| | APKLLIYNTNNLEAGVPSRFSGSGSGTDYTFTISSLQPEDIATY | |
| | FCLQHRSSYTFGQGTKLEIK | |
| NOV567 | DIQMTQSPSSLSASVGDRVTITCRGSQSIGNSLNVVYQQKPGK | 417 |
| | APKRLIYSTSTLEYGVPSRFSGSGSGTEYTLTISSLQPEDFATY | |
| | YCLQYATYPYTFGQGTKLEIK | |
| NOV221 | DIQMTQSPSSLSASVGDRVTITCKSSQNIDKYLNVVYQQKPGK | 449 |
| | APKLLIYNTNNLEAGVPSRFSGSGSGTDYTFTISSLQPEDIATY | |
| | FCLQHRSGYTFGQGTKLEIK | |
| CD3_sp11a_bkm1 | DIVMTQTPLSSPVTLGQPASISCRSSQSLVRSDGTTYFNVVLQ | 491 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11a_bkm2 | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_hz0 | DIVMTQTPLSSPVTLGQPASISCRSSQSLVRSDGTTYFNVVLQ | 491 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A_HZ1 | DIVMTQTPLSSPVTLGQPASISCRSSQSLVRSDGTTYFNVVYQ | 492 |
| | QRPGQPPRLLIYRVSNRFSGVPDRFSGSGAGTDFTLKISRVEA | |
| | EDVGVYYCLQSSH | |
| CD3_sp11a_sansPTM_hz1 | DIVMTQTPLSSPVTLGQPASISCRSSQSLVRSEGTTYFNVVYQ | 493 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_sansPTM_rat | DILVTQTPVSLPVSLGGHVSISCRSSQSLVRSEGTTYFNVVYLQ | 489 |
| | KPGQSPQLLIYRVSNRFSGVPDRFSGSGSGTDFTLKISRVEPE | |
| | DLGVYYCLQSSHFPVVTFGGGTKLELK | |
| CD3_sp11a_VHVL_YY | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VHVL_SS | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VHVL_WS | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SW | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VHVL_TT | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VHVL_TW | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VHVL_VVT | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A VH3_VLK_3 | EIVLTQSPGTLSLSPGERATLSCRSSQSLVRSEGTTYFNVVYQ | 494 |
| | QKPGQAPRLLIYRVSNRFSGIPDRFSGSGSGTDFTLTISRLEPE | |
| | DLAVYYCLQSSHFPVVTFGGGTKVEIK | |
| CD3_sp11a_VH1_VK2 | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH3_VLK1 | DIQMTQSPSSLSASVGDRVTITCRSSQSLVRSEGTTYFNVVYQ | 490 |
| | QKPGKAPKLLIYRVSNRFSGVPSRFSGSGSGTDFTLTISSLQP | |
| | EDFATYYCLQSSHFPVVTFGGGTKVEIK | |
| CD3_SP11A_VH5_VK2 | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp9aFW1_VL_VH_ | EIVMTQSPSTLSASVGDRVIITCKASQNINNYLNVVYQQKPGKA | 495 |
| S56G | PKLLIYNTDHLQAGVPSRFSGSGSGAEFTLTISSLQPDDFATYY | |
| | CLQHRSRYTFGQGTKLTVL | |
| CD3_SP9AFW4_VL_VH_ | EIVMTQSPSTLSASVGDRVIITCKASQNINNYLNVVYQQKPGKA | 495 |
| S56G | PKLLIYNTDHLQAGVPSRFSGSGSGAEFTLTISSLQPDDFATYY | |
| | CLQHRSRYTFGQGTKLTVL | |
| CD3_sp9aFW1_VLVH | EIVMTQSPSTLSASVGDRVIITCKASQNINNYLNVVYQQKPGKA | 495 |
| | PKLLIYNTDHLQAGVPSRFSGSGSGAEFTLTISSLQPDDFATYY | |
| | CLQHRSRYTFGQGTKLTVL | |
| CD3_sp9aFW4_VLVH | EIVMTQSPSTLSASVGDRVIITCKASQNINNYLNVVYQQKPGKA | 495 |
| | PKLLIYNTDHLQAGVPSRFSGSGSGAEFTLTISSLQPDDFATYY | |
| | CLQHRSRYTFGQGTKLTVL | |
| CD3_sp9arabtor_VHVL | EIVMTQSPSTLSASVGDRVIITCKASQNINNYLNVVYQQKPGKA | 495 |
| | PKLLIYNTDHLQAGVPSRFSGSGSGAEFTLTISSLQPDDFATYY | |
| | CLQHRSRYTFGQGTKLTVL | |
| CD3_sp9arabtor_VLVH | EIVMTQSPSTLSASVGDRVIITCKASQNINNYLNVVYQQKPGKA | 495 |
| | PKLLIYNTDHLQAGVPSRFSGSGSGAEFTLTISSLQPDDFATYY | |
| | CLQHRSRYTFGQGTKLTVL | |
| CD3_sp11a_VHVL_YY_ | DIVMTQTPLSSPVTLGQPASISCRSSQSLVRSEGTTYFNVVYQ | 493 |
| SANSPTM | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_YY_ | DIVMTQTPLSSPVTLGQPASISCRSSQSLVRSEGTTYFNVVYQ | 493 |
| SANSPTM_Y | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_YY_ | DIVMTQTPLSSPVTLGQPASISCRSSQSLVRSEGTTYFNVVYQ | 493 |
| SANSPTM_S | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_YY_Y | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_YY_S | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SS_ | DIVMTQTPLSSPVTLGQPASISCRSSQSLVRSEGTTYFNVVYQ | 493 |
| SANSPTM | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SS_ | DIVMTQTPLSSPVTLGQPASISCRSSQSLVRSEGTTYFNVVYQ | 493 |
| SANSPTM_Y | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SS_ | DIVMTQTPLSSPVTLGQPASISCRSSQSLVRSEGTTYFNVVYQ | 493 |
| SANSPTM_S | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SS_Y | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SS_S | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SS_ | DIVMTQTPLSSPVTLGQPASISCRSSQSLVRSEGTTYFNVVYQ | 493 |
| SANSPTM | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_WS_ | DIVMTQTPLSSPVTLGQPASISCRSSQSLVRSEGTTYFNVVYQ | 493 |
| SANSPTM_Y | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_WS_ | DIVMTQTPLSSPVTLGQPASISCRSSQSLVRSEGTTYFNVVYQ | 493 |
| SANSPTM_S | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_WS_Y | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_WS_S | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_WS_ | DIVMTQTPLSSPVTLGQPASISCRSSQSLVRSEGTTYFNVVYQ | 493 |
| SANSPTM | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SW_ | DIVMTQTPLSSPVTLGQPASISCRSSQSLVRSEGTTYFNVVYQ | 493 |
| SANSPTM_Y | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SW_ | DIVMTQTPLSSPVTLGQPASISCRSSQSLVRSEGTTYFNVVYQ | 493 |
| SANSPTM _S | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SW_Y | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11 a_VHVL_SW_S | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_SW_ | DIVMTQTPLSSPVTLGQPASISCRSSQSLVRSEGTTYFNVVYQ | 493 |
| SANSPTM | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_TW_ | DIVMTQTPLSSPVTLGQPASISCRSSQSLVRSEGTTYFNVVYQ | 493 |
| SANSPTM _Y | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_TW_ | DIVMTQTPLSSPVTLGQPASISCRSSQSLVRSEGTTYFNVVYQ | 493 |
| SANSPTM _S | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_TW_Y | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_TW_S | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_TW_ | DIVMTQTPLSSPVTLGQPASISCRSSQSLVRSEGTTYFNVVYQ | 493 |
| SANSPTM | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_TT_ | DIVMTQTPLSSPVTLGQPASISCRSSQSLVRSEGTTYFNVVYQ | 493 |
| SANSPTM_Y | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_TT_S | DIVMTQTPLSSPVTLGQPASISCRSSQSLVRSEGTTYFNVVYQ | 493 |
| ANSPTM_S | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_TT_Y | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VHVL_TT_S | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11 a_VHVL_TT_S | DIVMTQTPLSSPVTLGQPASISCRSSQSLVRSEGTTYFNVVYQ | 493 |
| ANSPTM | QRPGQPPRLLIYRVSNRFSGVPDRFSGSGAGTDFTLKISRVEA | |
| | EDVGVYYCLQSSHFPVVTFGGGTKVEIK | |
| CD3_SP11AVH3_VLK_3_Y | EIVLTQSPGTLSLSPGERATLSCRSSQSLVRSEGTTYFNVVYQ | 494 |
| | QKPGQAPRLLIYRVSNRFSGIPDRFSGSGSGTDFTLTISRLEPE | |
| | DLAVYYCLQSSHFPVVTFGGGTKVEIK | |
| CD3_SP11AVH3_VLK_3_S | EIVLTQSPGTLSLSPGERATLSCRSSQSLVRSEGTTYFNVVYQ | 494 |
| | QKPGQAPRLLIYRVSNRFSGIPDRFSGSGSGTDFTLTISRLEPE | |
| | DLAVYYCLQSSHFPVVTFGGGTKVEIK | |
| CD3_SP11AVH3_VLK_3_ | EIVLTQSPGTLSLSPGERATLSCRSSQSLVRSDGTTYFNVVYQ | 556 |
| Y_PTM | QKPGQAPRLLIYRVSNRFSGIPDRFSGSGSGTDFTLTISRLEPE | |
| | DLAVYYCLQSSHFPVVTFGGGTKVEIK | |
| CD3_SP11AVH3_VLK_3_ | EIVLTQSPGTLSLSPGERATLSCRSSQSLVRSDGTTYFNVVYQ | 556 |
| S_PTM | QKPGQAPRLLIYRVSNRFSGIPDRFSGSGSGTDFTLTISRLEPE | |
| | DLAVYYCLQSSHFPVVTFGGGTKVEIK | |
| CD3_SP11AVH3_VLK_3_ | EIVLTQSPGTLSLSPGERATLSCRSSQSLVRSEGTTYFNVVYQ | 494 |
| Y_SW | QKPGQAPRLLIYRVSNRFSGIPDRFSGSGSGTDFTLTISRLEPE | |
| | DLAVYYCLQSSHFPVVTFGGGTKVEIK | |
| CD3_SP11AVH3_VLK_3_ | EIVLTQSPGTLSLSPGERATLSCRSSQSLVRSEGTTYFNVVYQ | 494 |
| S_SW | QKPGQAPRLLIYRVSNRFSGIPDRFSGSGSGTDFTLTISRLEPE | |
| | DLAVYYCLQSSHFPVVTFGGGTKVEIK | |
| CD3_SP11AVH3_VLK_3_ | EIVLTQSPGTLSLSPGERATLSCRSSQSLVRSDGTTYFNVVYQ | 556 |
| Y_PTM_SW | QKPGQAPRLLIYRVSNRFSGIPDRFSGSGSGTDFTLTISRLEPE | |
| | DLAVYYCLQSSHFPVVTFGGGTKVEIK | |
| CD3_SP11AVH3_VLK_3_ | EIVLTQSPGTLSLSPGERATLSCRSSQSLVRSDGTTYFNVVYQ | 556 |
| S_SVVPTM | QKPGQAPRLLIYRVSNRFSGIPDRFSGSGSGTDFTLTISRLEPE | |
| | DLAVYYCLQSSHFPVVTFGGGTKVEIK | |
| CD3_SP11AVH3_VLK_S | EIVLTQSPGTLSLSPGERATLSCRSSQSLVRSDGTTYFNVVYQ | 556 |
| VVPTM | QKPGQAPRLLIYRVSNRFSGIPDRFSGSGSGTDFTLTISRLEPE | |
| | DLAVYYCLQSSHFPVVTFGGGTKVEIK | |
| CD3_SP11AVH3_VLK_3_ | EIVLTQSPGTLSLSPGERATLSCRSSQSLVRSEGTTYFNVVYQ | 494 |
| SW | QKPGQAPRLLIYRVSNRFSGIPDRFSGSGSGTDFTLTISRLEPE | |
| | DLAVYYCLQSSHFPVVTFGGGTKVEIK | |
| CD3_sp11a_VH1_VK2_Y | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VH1_VK2_S | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VH1_VK2_Y_ | DIVMTQTPLSSPVTLGQPASISCRSSQSLVRSEGTTYFNVVYQ | 493 |
| PTM | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VH1_VK2_S_ | DIVMTQTPLSSPVTLGQPASISCRSSQSLVRSEGTTYFNVVYQ | 493 |
| PTM | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VH1_VK2_Y_ | DIVMTQTPLSSPVTLGQPASISCRSSQSLVRSDGTTYFNVVYQ | 161 |
| SW | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VH1_VK2_S_ | DIVMTQTPLSSPVTLGQPASISCRSSQSLVRSDGTTYFNVVYQ | 161 |
| SW | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VH1_VK2_Y_ | DIVMTQTPLSSPVTLGQPASISCRSSQSLVRSEGTTYFNVVYQ | 493 |
| PTM | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VH1_VK2_S_ | DIVMTQTPLSSPVTLGQPASISCRSSQSLVRSEGTTYFNVVYQ | 493 |
| PTM_SW | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VH1_VK2_SW | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_sp11a_VH1_VK2_SW | DIVMTQTPLSSPVTLGQPASISCRSSQSLVRSEGTTYFNVVYQ | 493 |
| PTM | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH3_VLK1_Y | DIQMTQSPSSLSASVGDRVTITCRSSQSLVRSEGTTYFNVVYQ | 490 |
| | QKPGKAPKLLIYRVSNRFSGVPSRFSGSGSGTDFTLTISSLQP | |
| | EDFATYYCLQSSHFPVVTFGGGTKVEIK | |
| CD3_SP11A_VH3_VLK1_S | DIQMTQSPSSLSASVGDRVTITCRSSQSLVRSEGTTYFNVVYQ | 490 |
| | QKPGKAPKLLIYRVSNRFSGVPSRFSGSGSGTDFTLTISSLQP | |
| | EDFATYYCLQSSHFPVVTFGGGTKVEIK | |
| CD3_SP11A_VH3_VLK1_ | DIQMTQSPSSLSASVGDRVTITCRSSQSLVRSDGTTYFNVVYQ | 555 |
| Y_PTM | QKPGKAPKLLIYRVSNRFSGVPSRFSGSGSGTDFTLTISSLQP | |
| | EDFATYYCLQSSHFPVVTFGGGTKVEIK | |
| CD3 SP11A_VH3_VLK1_ | DIQMTQSPSSLSASVGDRVTITCRSSQSLVRSDGTTYFNVVYQ | 555 |
| S_PTM | QKPGKAPKLLIYRVSNRFSGVPSRFSGSGSGTDFTLTISSLQP | |
| | EDFATYYCLQSSHFPVVTFGGGTKVEIK | |
| CD3_SP11A_VH3_VLK1_ | DIQMTQSPSSLSASVGDRVTITCRSSQSLVRSEGTTYFNVVYQ | 490 |
| Y_SW | QKPGKAPKLLIYRVSNRFSGVPSRFSGSGSGTDFTLTISSLQP | |
| | EDFATYYCLQSSHFPVVTFGGGTKVEIK | |
| CD3_SP11A_VH3_VLK1_ | DIQMTQSPSSLSASVGDRVTITCRSSQSLVRSEGTTYFNVVYQ | 490 |
| S_SW | QKPGKAPKLLIYRVSNRFSGVPSRFSGSGSGTDFTLTISSLQP | |
| | EDFATYYCLQSSHFPVVTFGGGTKVEIK | |
| CD3_SP11A_VH3_VLK1_ | DIQMTQSPSSLSASVGDRVTITCRSSQSLVRSDGTTYFNVVYQ | 555 |
| Y_PTM | QKPGKAPKLLIYRVSNRFSGVPSRFSGSGSGTDFTLTISSLQP | |
| | EDFATYYCLQSSHFPVVTFGGGTKVEIK | |
| CD3_SP11A_VH3_VLK1 | DIQMTQSPSSLSASVGDRVTITCRSSQSLVRSDGTTYFNVVYQ | 555 |
| S_PTM_SW | QKPGKAPKLLIYRVSNRFSGVPSRFSGSGSGTDFTLTISSLQP | |
| | EDFATYYCLQSSHFPVVTFGGGTKVEIK | |
| CD3_SP11A_VH3_VLK1_ | DIQMTQSPSSLSASVGDRVTITCRSSQSLVRSDGTTYFNVVYQ | 555 |
| PTM_SW | QKPGKAPKLLIYRVSNRFSGVPSRFSGSGSGTDFTLTISSLQP | |
| | EDFATYYCLQSSHFPVVTFGGGTKVEIK | |
| CD3 SP11A_VH3_VLK1_ | DIQMTQSPSSLSASVGDRVTITCRSSQSLVRSEGTTYFNVVYQ | 490 |
| SW | QKPGKAPKLLIYRVSNRFSGVPSRFSGSGSGTDFTLTISSLQP | |
| | EDFATYYCLQSSHFPVVTFGGGTKVEIK | |
| CD3_SP11A_VH5_VK2_Y | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH5_VK2_S | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH5_VK2_ | DIVMTQTPLSSPVTLGQPASISCRSSQSLVRSDGTTYFNVVYQ | 161 |
| Y_PTM | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH5_VK2_ | DIVMTQTPLSSPVTLGQPASISCRSSQSLVRSDGTTYFNVVYQ | 161 |
| S_PTM | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH5_VK2_ | DIVMTQTPLSSPVTLGQPASISCRSSQSLVRSDGTTYFNVVYQ | 161 |
| Y_SW | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH5_VK2_ | DIVMTQTPLSSPVTLGQPASISCRSSQSLVRSDGTTYFNVVYQ | 161 |
| S_SW | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH5_VK2_ | DIVMTQTPLSSPVTLGQPASISCRSSQSLVRSDGTTYFNVVYQ | 161 |
| Y_PTM_SW | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH5_VK2_ | DIVMTQTPLSSPVTLGQPASISCRSSQSLVRSDGTTYFNVVYQ | 161 |
| S_PTM_SW | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH5_VK2_ | DIVMTQTPLSSPVTLGQPASISCRSSQSLVRSDGTTYFNVVYQ | 161 |
| PTM_SW | QRPGQPPRLLIYRVSNRFSGVPDRFSGSGAGTDFTLKISRVEA | |
| | EDVGVYYCLQSSHFPVVTFGGGTKVEIK | |
| CD3_SP11A_VH5_VK2_SW | DIVMTQTPLSSPVTLGQPASISCRSSQSLVRSDGTTYFNVVYQ | 161 |
| | QRPGQPPRLLIYRVSNRFSGVPDRFSGSGAGTDFTLKISRVEA | |
| | EDVGVYYCLQSSHFPVVTFGGGTKVEIK | |

Tables 1A to 10 list CDR consensus sequences based on the CDR sequences of the exemplary CD3 binding molecules described herein. The group C1 CDR sequences in Table 1A are based upon the Kabat CDR sequences, Chothia CDR sequences, IMGT CDR sequences, and combinations thereof, of the exemplary CD3 binding molecules NOV292, NOV589, NOV567, and the exemplary CD3 binding molecules which include “sp11a” in the binder name. The group C2 CDR sequences in Table 1B are based upon the Kabat CDR sequences, Chothia CDR sequences, IMGT CDR sequences, and combinations thereof, of the exemplary CD3 binding molecules NOV453, NOV229, NOV580, NOV221, and the exemplary CD3 binding molecules which include “sp9a” in the binder name. The group C3 CDR sequences in Table 10 are based upon the Kabat CDR sequences, Chothia CDR sequences, IMGT CDR sequences, and combinations thereof, of the exemplary CD3 binding molecules NOV123, sp10b, NOV110, and NOV832.

The specific CDR sequences of the exemplary CD3 binding molecules described in the Examples are listed in Tables 1B-1 to 1H-2. Exemplary VH and VL sequences are listed in Tables 1J-1 and 1J-2, respectively.

In some embodiments, the CD3 binding molecules comprise a heavy chain CDR having an amino acid sequence of any one of the CDR consensus sequences listed in Table 1A, Table 1B, or Table 10. In particular embodiments, the present disclosure provides CD3 binding molecules, comprising (or alternatively, consisting of) one, two, three, or more heavy chain CDRs selected from the heavy chain CDRs described in Table 1A, Table 1B, or Table 10.

In some embodiments, the CD3 binding molecules comprise a light chain CDR having an amino acid sequence of any one of the CDR consensus sequences listed in Table 1A, Table 1B, or Table 10. In particular embodiments, the present disclosure provides CD3 binding molecules, comprising (or alternatively, consisting of) one, two, three, or more light chain CDRs selected from the light chain CDRs described in Table 1A, Table 1B, or Table 10.

In some embodiments, a CD3 binding molecule comprises a CDR-H1 sequence, a CDR-H2 sequence a CDR-H3 sequence, a CDR-L1 sequence, a CDR-L2 sequence, and a CDR-L3 sequence set forth in Table 1A.

In some embodiments, the amino acid designated X₁ in Table 1A is T. In some embodiments, the amino acid designated X₁ in Table 1A is A. In some embodiments, the amino acid designated X₂ in Table 1A is S. In some embodiments, the amino acid designated X₂ in Table 1A is R. In some embodiments, the amino acid designated X₃ in Table 1A is N. In some embodiments, the amino acid designated X₃ in Table 1A is Y. In some embodiments, the amino acid designated X₃ in Table 1A is Q. In some embodiments, the amino acid designated X₄ in Table 1A is H. In some embodiments, the amino acid designated X₄ in Table 1A is S. In some embodiments, the amino acid designated X₅ in Table 1A is M. In some embodiments, the amino acid designated X₅ in Table 1A is L. In some embodiments, the amino acid designated X₆ in Table 1A is K. In some embodiments, the amino acid designated X₆ in Table 1A is R. In some embodiments, the amino acid designated X₇ in Table 1A is S. In some embodiments, the amino acid designated X₇ in Table 1A is K. In some embodiments, the amino acid designated X₅₅ in Table 1A is F. In some embodiments, the amino acid designated X₅₅ in Table 1A is Y. In some embodiments, the amino acid designated X₅₅ in Table 1A is S. In some embodiments, the amino acid designated X₈ in Table 1A is W. In some embodiments, the amino acid designated X₈ in Table 1A is Y. In some embodiments, the amino acid designated X₈ in Table 1A is S. In some embodiments, the amino acid designated X₈ in Table 1A is T. In some embodiments, the amino acid designated X₉ in Table 1A is W. In some embodiments, the amino acid designated X₉ in Table 1A is Y. In some embodiments, the amino acid designated X₉ in Table 1A is S. In some embodiments, the amino acid designated X₉ in Table 1A is T. In some embodiments, the amino acid designated X₁₀ in Table 1A is H. In some embodiments, the amino acid designated X₁₀ in Table 1A is Y. In some embodiments, the amino acid designated X₁₁ in Table 1A is S. In some embodiments, the amino acid designated X₁₁ in Table 1A is G. In some embodiments, the amino acid designated X₁₂ in Table 1A is I. In some embodiments, the amino acid designated X₁₂ in Table 1A is L. In some embodiments, the amino acid designated X₁₃ in Table 1A is V. In some embodiments, the amino acid designated X₁₃ in Table 1A is G. In some embodiments, the amino acid designated X₁₄ in Table 1A is R. In some embodiments, the amino acid designated X₁₄ in Table 1A is N. In some embodiments, the amino acid designated X₁₅ in Table 1A is D. In some embodiments, the amino acid designated X₁₅ in Table 1A is E. In some embodiments, the amino acid designated X₁₅ in Table 1A is L. In some embodiments, the amino acid designated X₁₆ in Table 1A is G. In some embodiments, the amino acid designated X₁₆ in Table 1A is N. In some embodiments, the amino acid designated X₁₆ in Table 1A is E. In some embodiments, the amino acid designated X₁₇ in Table 1A is R. In some embodiments, the amino acid designated X₁₇ in Table 1A is S. In some embodiments, the amino acid designated X₁₈ in Table 1A is V. In some embodiments, the amino acid designated X₁₈ in Table 1A is T. In some embodiments, the amino acid designated X₁₉ in Table 1A is N. In some embodiments, the amino acid designated X₁₉ in Table 1A is T. In some embodiments, the amino acid designated X₂₀ in Table 1A is R. In some embodiments, the amino acid designated X₂₀ in Table 1A is L. In some embodiments, the amino acid designated X₂₁ in Table 1A is F. In some embodiments, the amino acid designated X₂₁ in Table 1A is E. In some embodiments, the amino acid designated X₂₂ in Table 1A is S. In some embodiments, the amino acid designated X₂₂ in Table 1A is Y. In some embodiments, the amino acid designated X₂₃ in Table 1A is S. In some embodiments, the amino acid designated X₂₃ in Table 1A is Y. In some embodiments, the amino acid designated X₂₄ in Table 1A is S. In some embodiments, the amino acid designated X₂₄ in Table 1A is A. In some embodiments, the amino acid designated X₂₅ in Table 1A is H. In some embodiments, the amino acid designated X₂₅ in Table 1A is T. In some embodiments, the amino acid designated X₂₆ in Table 1A is F. In some embodiments, the amino acid designated X₂₆ in Table 1A is Y. In some embodiments, the amino acid designated X₂₇ in Table 1A is W. In some embodiments, the amino acid designated X₂₇ in Table 1A is Y.

In some embodiments, a CD3 binding molecule comprises the CDR-H1 sequence C1-1. In some embodiments, a CD3 binding molecule comprises the CDR-H1 sequence C1-2. In some embodiments, a CD3 binding molecule comprises the CDR-H1 sequence C1-3. In some embodiments, a CD3 binding molecule comprises the CDR-H1 sequence C1-4.

In some embodiments, a CD3 binding molecule comprises the CDR-H2 sequence C1-5. In some embodiments, a CD3 binding molecule comprises the CDR-H2 sequence C1-6. In some embodiments, a CD3 binding molecule comprises the CDR-H2 sequence C1-7.

In some embodiments, a CD3 binding molecule comprises the CDR-H3 sequence C1-8. In some embodiments, a CD3 binding molecule comprises the CDR-H3 sequence C1-9. In some embodiments, a CD3 binding molecule comprises the CDR-H3 sequence C1-10. In some embodiments, a CD3 binding molecule comprises the CDR-H3 sequence C1-11.

In some embodiments, a CD3 binding molecule comprises the CDR-L1 sequence C1-12. In some embodiments, a CD3 binding molecule comprises the CDR-L1 sequence C1-13. In some embodiments, a CD3 binding molecule comprises the CDR-L1 sequence C1-14. In some embodiments, a CD3 binding molecule comprises the CDR-L1 sequence C1-15. In some embodiments, a CD3 binding molecule comprises the CDR-L1 sequence C1-16. In some embodiments, a CD3 binding molecule comprises the CDR-L1 sequence C1-17.

In some embodiments, a CD3 binding molecule comprises the CDR-L2 sequence C1-18. In some embodiments, a CD3 binding molecule comprises the CDR-L2 sequence C1-19.

In some embodiments, a CD3 binding molecule comprises the CDR-L3 sequence C1-20. In some embodiments, a CD3 binding molecule comprises the CDR-L3 sequence C1-21. In some embodiments, a CD3 binding molecule comprises the CDR-L3 sequence C1-22. In some embodiments, a CD3 binding molecule comprises the CDR-L3 sequence C1-23.

In some embodiments, a CD3 binding molecule comprises a CDR-H1 sequence, a CDR-H2 sequence a CDR-H3 sequence, a CDR-L1 sequence, a CDR-L2 sequence, and a CDR-L3 sequence set forth in Table 1B.

In some embodiments, the amino acid designated X₂₈ in Table 1B is V. In some embodiments, the amino acid designated X₂₈ in Table 1B is I. In some embodiments, the amino acid designated X₂₉ in Table 1B is F. In some embodiments, the amino acid designated X₂₉ in Table 1B is Y. In some embodiments, the amino acid designated X₃₀ in Table 1B is N. In some embodiments, the amino acid designated X₃₀ in Table 1B is S. In some embodiments, the amino acid designated X₃₁ in Table 1B is A. In some embodiments, the amino acid designated X₃₁ in Table 1B is S. In some embodiments, the amino acid designated X₃₂ in Table 1B is T. In some embodiments, the amino acid designated X₃₂ in Table 1B is K. In some embodiments, the amino acid designated X₃₃ in Table 1B is T. In some embodiments, the amino acid designated X₃₃ in Table 1B is A. In some embodiments, the amino acid designated X₃₄ in Table 1B is S. In some embodiments, the amino acid designated X₃₄ in Table 1B is R. In some embodiments, the amino acid designated X₃₅ in Table 1B is N. In some embodiments, the amino acid designated X₃₅ in Table 1B is G. In some embodiments, the amino acid designated X₃₆ in Table 1B is S. In some embodiments, n the amino acid designated X₃₆ in Table 1B is A. In some embodiments, the amino acid designated X₃₇ in Table 1B is A. In some embodiments, the amino acid designated X₃₇ in Table 1B is T. In some embodiments, the amino acid designated X₃₇ in Table 1B is S. In some embodiments, the amino acid designated X₃₈ in Table 1B is N. In some embodiments, the amino acid designated X₃₈ in Table 1B is D. In some embodiments, the amino acid designated X₃₉ in Table 1B is N. In some embodiments, the amino acid designated X₃₉ in Table 1B is K. In some embodiments, the amino acid designated X₄₀ in Table 1B is D. In some embodiments, the amino acid designated X₄₀ in Table 1B is N. In some embodiments, the amino acid designated X₄₁ in Table 1B is H. In some embodiments, the amino acid designated X₄₁ in Table 1B is N. In some embodiments, the amino acid designated X₄₂ in Table 1B is Q. In some embodiments, the amino acid designated X₄₂ in Table 1B is E. In some embodiments, the amino acid designated X₄₃ in Table 1B is R. In some embodiments, the amino acid designated X₄₃ in Table 1B is S. In some embodiments, the amino acid designated X₄₃ in Table 1B is G. In some embodiments, a CD3 binding molecule comprises the CDR-H1 sequence C2-1. In some embodiments, a CD3 binding molecule comprises the CDR-H1 sequence C2-2.

In some embodiments, a CD3 binding molecule comprises the CDR-H1 sequence C2-3. In some embodiments, a CD3 binding molecule comprises the CDR-H1 sequence C2-4.

In some embodiments, a CD3 binding molecule comprises the CDR-H2 sequence C2-5. In some embodiments, a CD3 binding molecule comprises the CDR-H2 sequence C2-6. In some embodiments, a CD3 binding molecule comprises the CDR-H2 sequence C2-7.

In some embodiments, a CD3 binding molecule comprises the CDR-H3 sequence C2-8. In some embodiments, a CD3 binding molecule comprises the CDR-H3 sequence C2-9.

In some embodiments, a CD3 binding molecule comprises the CDR-L1 sequence C2-10. In some embodiments, a CD3 binding molecule comprises the CDR-L1 sequence C2-11. In some embodiments, a CD3 binding molecule comprises the CDR-L1 sequence C2-12.

In some embodiments, a CD3 binding molecule comprises the CDR-L2 sequence C2-13. In some embodiments, a CD3 binding molecule comprises the CDR-L2 sequence C2-14. In some embodiments, a CD3 binding molecule comprises the CDR-L2 sequence C2-15.

In some embodiments, a CD3 binding molecule comprises the CDR-L3 sequence C2-16. In some embodiments, a CD3 binding molecule comprises the CDR-L3 sequence C2-17.

In some embodiments, a CD3 binding molecule comprises a CDR-H1 sequence, a CDR-H2 sequence a CDR-H3 sequence, a CDR-L1 sequence, a CDR-L2 sequence, and a CDR-L3 sequence set forth in Table 10.

In some embodiments, the amino acid designated X₄₄ in Table 10 is G. In some embodiments, the amino acid designated X₄₄ in Table 10 is A. In some embodiments, the amino acid designated X₄₅ in Table 10 is H. In some embodiments, the amino acid designated X₄₅ in Table 10 is N. In some embodiments, the amino acid designated X₄₆ in Table 10 is D. In some embodiments, the amino acid designated X₄₆ in Table 10 is G. In some embodiments, the amino acid designated X₄₇ in Table 10 is A. In some embodiments, the amino acid designated X₄₇ in Table 10 is G. In some embodiments, the amino acid designated X₄₈ in Table 10 is N. In some embodiments, the amino acid designated X₄₈ in Table 10 is K. In some embodiments, the amino acid designated X₄₉ in Table 10 is V. In some embodiments, the amino acid designated X₄₉ in Table 10 is A. In some embodiments, the amino acid designated X₅₀ in Table 10 is N. In some embodiments, the amino acid designated X₅₀ in Table 10 is V. In some embodiments, the amino acid designated X₅₁ in Table 10 is A. In some embodiments, the amino acid designated X₅₁ in Table 10 is V. In some embodiments, the amino acid designated X₅₂ in Table 10 is Y. In some embodiments, the amino acid designated X₅₂ in Table 10 is F. In some embodiments, the amino acid designated X₅₃ in Table 10 is I. In some embodiments, the amino acid designated X₅₃ in Table 10 is V. In some embodiments, the amino acid designated X₅₄ in Table 10 is I. In some embodiments, the amino acid designated X₅₄ in Table 10 is H.

In some embodiments, a CD3 binding molecule comprises the CDR-H1 sequence C3-1. In some embodiments, a CD3 binding molecule comprises the CDR-H1 sequence C3-2. In some embodiments, a CD3 binding molecule comprises the CDR-H1 sequence C3-3. In some embodiments, a CD3 binding molecule comprises the CDR-H1 sequence C3-4.

In some embodiments, a CD3 binding molecule comprises the CDR-H2 sequence C3-5. In some embodiments, a CD3 binding molecule comprises the CDR-H2 sequence C3-6. In some embodiments, a CD3 binding molecule comprises the CDR-H2 sequence C3-7.

In some embodiments, a CD3 binding molecule comprises the CDR-H3 sequence C3-8. In some embodiments, a CD3 binding molecule comprises the CDR-H3 sequence C3-9.

In some embodiments, a CD3 binding molecule comprises the CDR-L1 sequence C3-10. In some embodiments, a CD3 binding molecule comprises the CDR-L1 sequence C3-11. In some embodiments, a CD3 binding molecule comprises the CDR-L1 sequence C3-12.

In some embodiments, a CD3 binding molecule comprises the CDR-L2 sequence C3-13. In some embodiments, a CD3 binding molecule comprises the CDR-L2 sequence C3-14.

In some embodiments, a CD3 binding molecule comprises the CDR-L3 sequence C3-15. In some embodiments, a CD3 binding molecule comprises the CDR-L3 sequence C3-16.

In some embodiments, a CD3 binding molecule comprises CDR-H1, CDR-H2, and CDR-H3 sequences set forth in Table 1D-1 and the corresponding CDR-L1, CDR-L2, and CDR-L3 sequences set forth in Table 1D-2.

In some embodiments, a CD3 binding molecule comprises CDR-H1, CDR-H2, and CDR-H3 sequences set forth in Table 1E-1 and the corresponding CDR-L1, CDR-L2, and CDR-L3 sequences set forth in Table 1E-2.

In some embodiments, a CD3 binding molecule comprises CDR-H1, CDR-H2, and CDR-H3 sequences set forth in Table 1F-1 and the corresponding CDR-L1, CDR-L2, and CDR-L3 sequences set forth in Table 1F-2.

In some embodiments, a CD3 binding molecule comprises CDR-H1, CDR-H2, and CDR-H3 sequences set forth in Table 1G-1 and the corresponding CDR-L1, CDR-L2, and CDR-L3 sequences set forth in Table 1G-2.

In some embodiments, a CD3 binding molecule comprises CDR-H1, CDR-H2, and CDR-H3 sequences set forth in Table 1H-1 and the corresponding CDR-L1, CDR-L2, and CDR-L3 sequences set forth in Table 1H-2.

In some embodiments, a CD3 binding molecule comprises CDR-H1, CDR-H2, and CDR-H3 sequences set forth in Table 1I-1 and the corresponding CDR-L1, CDR-L2, and CDR-L3 sequences set forth in Table 1I-2.

In some embodiments, a CD3 binding molecule comprises a heavy chain CDR having an amino acid sequence of any one of the CDRs listed in Table 1B-1, Table 1C-1, Table 1D-1, Table 1E-1, Table 1F-1, Table 1G-1, Table 1H-1, or Table 1I-1. In particular embodiments, the present disclosure provides CD3 binding molecules, comprising (or alternatively, consisting of) one, two, three, or more heavy chain CDRs selected the heavy chain CDRs described in Table 1B-1, Table 1C-1, Table 1D-1, Table 1E-1, Table 1F-1, Table 1G-1, Table 1H-1, and Table 1I-1.

In some embodiments, a CD3 binding molecule comprises a light chain CDR having an amino acid sequence of any one of the CDRs listed in Table 1B-2, Table 1C-2, Table 1D-2, Table 1E-2, Table 1F-2, Table 1G-2, Table 1H-2, or Table 1I-2. In particular embodiments, the present disclosure provides CD3 binding molecules, comprising (or alternatively, consisting of) one, two, three, or more light chain CDRs selected the light chain CDRs described in Table 1B-2, Table 1C-2, Table 1D-2, Table 1E-2, Table 1F-2, Table 1G-2, Table 1H-2, and Table 1I-2.

Other CD3 binding molecules include amino acids that have been mutated, yet have at least 80, 85, 90, 95, 96, 97, 98, or 99 percent identity in the CDR regions with the CDR sequences described in Table 1. In some embodiments, such CD3 binding molecules include mutant amino acid sequences where no more than 1, 2, 3, 4 or 5 amino acids have been mutated in the CDR regions when compared with the CDR sequences described in Table 1.

In some embodiments, a CD3 binding molecule comprises a VH and/or VL domain having an amino acid sequence of any VH and/or VL domain described in Table 1. Other CD3 binding molecules include VH and/or VL domains comprising amino acid sequences having at least 80, 85, 90, 95, 96, 97, 98, or 99 percent identity to the VH and/or VL sequences described in Table 1. In some embodiments, CD3 binding molecules include VH and/or VL domains where no more than 1, 2, 3, 4 or 5 amino acids have been mutated when compared with the VH and/or VL domains depicted in the sequences described in Table 1, while retaining substantially the same therapeutic activity.

VH and VL sequences (amino acid sequences and the nucleotide sequences encoding the amino acid sequences) can be “mixed and matched” to create other CD3 binding molecules. Such “mixed and matched” CD3 binding molecules can be tested using binding assays known in the art (e.g., FACS assays described in the Examples). When chains are mixed and matched, a VH sequence from a particular VH/VL pairing should be replaced with a structurally similar VH sequence. A VL sequence from a particular VH/VL pairing should be replaced with a structurally similar VL sequence.

Accordingly, in one embodiment, the present disclosure provides CD3 binding molecules having: a heavy chain variable region (VH) comprising an amino acid sequence selected from any one of the VH sequences described in Table 1-J1; and a light chain variable region (VL) comprising an amino acid sequence described in Table 1-J2.

The CD3 binding molecules can be fused or chemically conjugated (including both covalent and non-covalent conjugations) to a heterologous protein or polypeptide (or fragment thereof, for example to a polypeptide of at least 10, at least 20, at least 30, at least 40, at least 50, at least 60, at least 70, at least 80, at least 90 or at least 100 amino acids). For example, a CD3 binding molecule can be fused directly or indirectly to a detectable protein, e.g., an enzyme or a fluorescent protein. Methods for fusing or conjugating proteins, polypeptides, or peptides to an antibody or an antibody fragment are known and can be used to fuse or conjugate a protein or polypeptide to a CD3 binding molecule of the disclosure. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88:10535-10539; Zheng et al., 1995, J. Immunol. 154:5590-5600; and Vil et al., 1992, Proc. Natl. Acad. Sci. USA 89:11337-11341.

Additional CD3 binding molecules can be generated through the techniques of gene-shuffling, motif-shuffling, exon-shuffling, and/or codon-shuffling (collectively referred to as “DNA shuffling”). DNA shuffling can be employed to alter the activities of molecules of the disclosure or fragments thereof (e.g., molecules or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-313. The CD3 binding molecules described herein or fragments thereof can be altered by being subjected to random mutagenesis by error-prone PCR, random nucleotide insertion or other methods prior to recombination. A polynucleotide encoding a fragment of a CD3 binding molecule described herein can be recombined with one or more components, motifs, sections, parts, domains, fragments, etc. of one or more heterologous molecules.

Moreover, CD3 binding molecules can be fused to marker sequences, such as a peptide to facilitate purification. In some embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984 Cell 37:767), and the “flag” tag.

### 7.3. Antigen Binding Modules

Typically, one or more ABMs of the MBMs comprise immunoglobulin-based antigen-binding domains, for example the sequences of antibody fragments or derivatives. These antibody fragments and derivatives typically include the CDRs of an antibody and can include larger fragments and derivatives thereof, e.g., Fabs, scFabs, Fvs, and scFvs.

Immunoglobulin-based ABMs can comprise modifications to framework residues within a VH and/or a VL, e.g. to improve the properties of a MBM containing the ABM. For example, framework modifications can be made to decrease immunogenicity of a MBM. One approach for making such framework modifications is to “back-mutate” one or more framework residues of the ABM to a corresponding germline sequence. Such residues can be identified by comparing framework sequences to germline sequences from which the ABM is derived. To “match” framework region sequences to desired germline configuration, residues can be “back-mutated”to a corresponding germline sequence by, for example, site-directed mutagenesis. MBMs having such “back-mutated” ABMs are intended to be encompassed by the disclosure.

Another type of framework modification involves mutating one or more residues within a framework region, or even within one or more CDR regions, to remove T-cell epitopes to thereby reduce potential immunogenicity of a MBM. This approach is also referred to as “deimmunization” and is described in further detail in U.S. Patent Publication 20030153043 by Carr et al.

ABMs can also be modified to have altered glycosylation, which can be useful, for example, to increase the affinity of a MBM for one or more of its antigens. Such carbohydrate modifications can be accomplished by, for example, altering one or more sites of glycosylation within an ABM sequence. For example, one or more amino acid substitutions can be made that result in elimination of one or more variable region framework glycosylation sites to thereby eliminate glycosylation at that site. Such aglycosylation can increase the affinity of the MBM for an antigen. Such an approach is described in, e.g., U.S. Pat. Nos. 5,714,350 and 6,350,861 by Co et al.

### 7.3.1. Immunoglobulin Based Modules

7.3.1.1. Fabs

In certain aspects, an ABM is a Fab domain. Fab domains can be produced by proteolytic cleavage of immunoglobulin molecules, using enzymes such as papain, or through recombinant expression. Fab domains typically comprise a CH1 domain attached to a VH domain which pairs with a CL domain attached to a VL domain.

In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the MBMs, it is advantageous to use Fab heterodimerization strategies to permit the correct association of Fab domains belonging to the same ABM and minimize aberrant pairing of Fab domains belonging to different ABMs. For example, the Fab heterodimerization strategies shown in Table 2 below can be used:

TABLE 2
Fab Heterodimerization Strategies

| Name | STRATEGY | VH | CH1 | VL | CL | REFERENCE |
|---|---|---|---|---|---|---|
| F1 | CrossMabCH1- | WT | CL domain | WT | CH1 domain | Schaefer et al., |
| | CL | | | | | 2011, Cancer Cell |
| | | | | | | 2011; 20: 472-86; |
| | | | | | | PMID: 22014573. |
| F2 | orthogonal Fab | 39K, | H172A, F174G | 1R, | L135Y, S176W | Lewis et al., 2014, |
| | VHVRD1CH1CRD2 - | 62E | | 38D, | | Nat Biotechnol |
| | VLVRD1CλCRD2 | | | (36F) | | 32: 191-8 |
| F3 | orthogonal Fab | 39Y | WT | 38R | WT | Lewis et al., 2014, |
| | VHVRD2CH1wt - | | | | | Nat Biotechnol |
| | VLVRD2Cλwt | | | | | 32: 191-8 |
| F4 | TCR CαCβ | 39K | TCR Cα | 38D | TCR Cβ | Wu et al., 2015, |
| | | | | | | MAbs 7: 364-76 |
| F5 | CR3 | WT | T192E | WT | N137K, S114A | Golay at al., 2016, J |
| | | | | | | Immunol 196: 3199-211. |
| F6 | MUT4 | WT | L143Q, S188V | WT | V133T, S176V | Golay at al., 2016, J |
| | | | | | | Immunol 196: 3199-211. |
| F7 | DuetMab | WT | F126C | WT | S121C | Mazor et al., 2015, |
| | | | | | | MAbs 7: 377-89; |
| | | | | | | Mazor et al., 2015, |
| | | | | | | MAbs 7: 461-669. |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179.

In some embodiments, the Fab domain comprises a 192E substitution in the CH1 domain and 114A and 137K substitutions in the CL domain, which introduces a salt-bridge between the CH1 and CL domains (see, Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121C in the CL domain (see, Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the α T cell receptor and substituting the CL domain with the β domain of the T cell receptor, and pairing these domain replacements with an additional charge-charge interaction between the VL and VH domains by introducing a 38D modification in the VL domain and a 39K modification in the VH domain.

ABMs can comprise a single chain Fab fragment, which is a polypeptide consisting of an antibody heavy chain variable domain (VH), an antibody constant domain 1 (CH1), an antibody light chain variable domain (VL), an antibody light chain constant domain (CL) and a linker. In some embodiments, the antibody domains and the linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. The linker can be a polypeptide of at least 30 amino acids, preferably between 32 and 50 amino acids. The single chain Fab domains are stabilized via the natural disulfide bond between the CL domain and the CH1 domain.

In an embodiment, the antibody domains and the linker in the single chain Fab fragment have one of the following orders in N-terminal to C-terminal direction: a) VH-CH1-linker-VL-CL, or b) VL-CL-linker-VH-CH1, more preferably VL-CL-linker-VH-CH1.

In another embodiment, the antibody domains and the linker in the single chain Fab fragment have one of the following orders in N-terminal to C-terminal direction: a) VH-CL-linker-VL-CH1 or b) VL-CH1-linker-VH-CL.

Optionally in the single chain Fab fragment, additionally to the natural disulfide bond between the CL-domain and the CH1 domain, also the antibody heavy chain variable domain (VH) and the antibody light chain variable domain (VL) are disulfide stabilized by introduction of a disulfide bond between the following positions: i) heavy chain variable domain position 44 to light chain variable domain position 100, ii) heavy chain variable domain position 105 to light chain variable domain position 43, or iii) heavy chain variable domain position 101 to light chain variable domain position 100 (numbering according to EU index of Kabat).

Such further disulfide stabilization of single chain Fab fragments is achieved by the introduction of a disulfide bond between the variable domains VH and VL of the single chain Fab fragments. Techniques to introduce unnatural disulfide bridges for stabilization for a single chain Fv are described e.g. in WO 94/029350, Rajagopal et al., 1997, Prot. Engin. 10:1453-59; Kobayashi et al., 1998, Nuclear Medicine & Biology, 25:387-393; and Schmidt, et al., 1999, Oncogene 18:1711-1721. In one embodiment the optional disulfide bond between the variable domains of the single chain Fab fragments is between heavy chain variable domain position 44 and light chain variable domain position 100. In one embodiment the optional disulfide bond between the variable domains of the single chain Fab fragments is between heavy chain variable domain position 105 and light chain variable domain position 43 (numbering according to EU index of Kabat).

7.3.1.2. scFv

Single chain Fv or “scFv” antibody fragments comprise the VH and VL domains of an antibody in a single polypeptide chain, are capable of being expressed as a single chain polypeptide, and retain the specificity of the intact antibody from which it is derived. Generally, the scFv polypeptide further comprises a polypeptide linker between the VH and VL domain that enables the scFv to form the desired structure for target binding. Examples of linkers suitable for connecting the VH and VL chains of a scFV are the ABM linkers identified in Section 7.4.3, for example any of the linkers designated L1 through L54.

Unless specified, as used herein an scFv can have the VL and VH variable regions in either order, e.g., with respect to the N-terminal and C-terminal ends of the polypeptide, the scFv can comprise VL-linker-VH or can comprise VH-linker-VL.

To create an scFv-encoding nucleic acid, the VH and VL-encoding DNA fragments are operably linked to another fragment encoding a linker, e.g., encoding any of the ABM linkers described in Section 7.4.3 (such as the amino acid sequence (Gly4^{˜}Ser)3), such that the VH and VL sequences can be expressed as a contiguous single-chain protein, with the VL and VH regions joined by the flexible linker (see e.g., Bird et al., 1988, Science 242:423-426; Huston et al., 1988, Proc. Natl. Acad. Sci. USA 85:5879-5883; McCafferty et al., 1990, Nature 348:552-554).

7.3.1.3. Other Immunoglobulin-Based Modules

MBMs can also comprise ABMs having an immunoglobulin format which is other than Fab or scFv, for example Fv, dsFv, (Fab′)2, a single domain antibody (SDAB), a VH or VL domain, or a camelid VHH domain (also called a nanobody).

An ABM can be a single domain antibody composed of a single VH or VL domain which exhibits sufficient affinity to the target. In a specific embodiment, the single domain antibody is a camelid VHH domain (see, e.g., Riechmann, 1999, Journal of Immunological Methods 231:25-38; WO 94/04678).

7.3.2. Non-Immunoglobulin Based Modules

In certain embodiments, one or more of the ABMs are derived from non-antibody scaffold proteins (including, but not limited to, designed ankyrin repeat proteins (DARPins), Avimers (short for avidity multimers), Anticalin/Lipocalins, Centyrins, Kunitz domains, Adnexins, Affilins, Affitins (also known as Nonfitins), Knottins, Pronectins, Versabodies, Duocalins, and Fynomers), ligands, receptors, cytokines or chemokines.

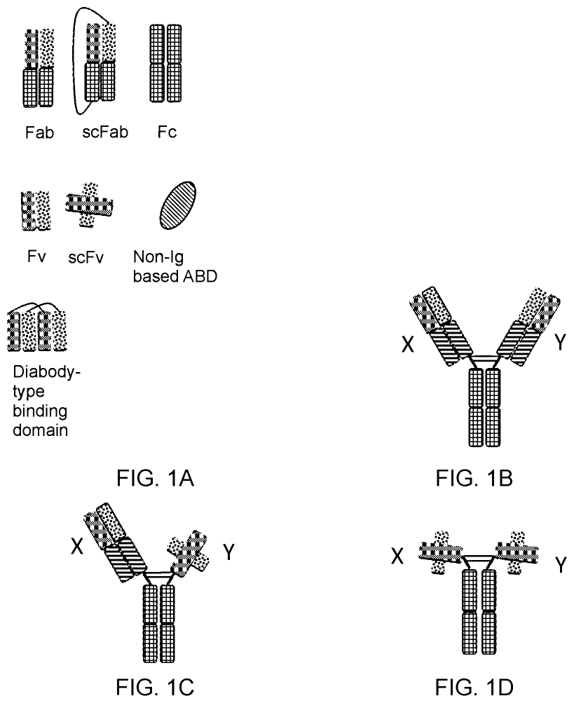

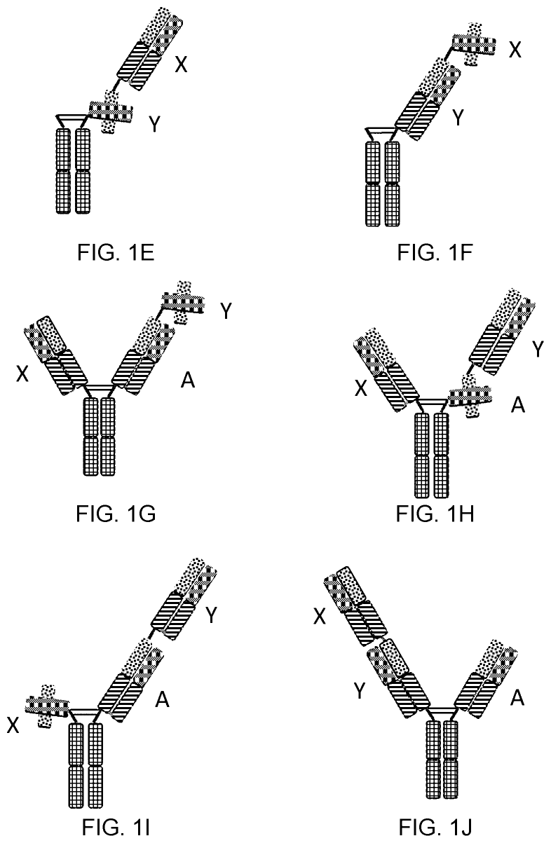

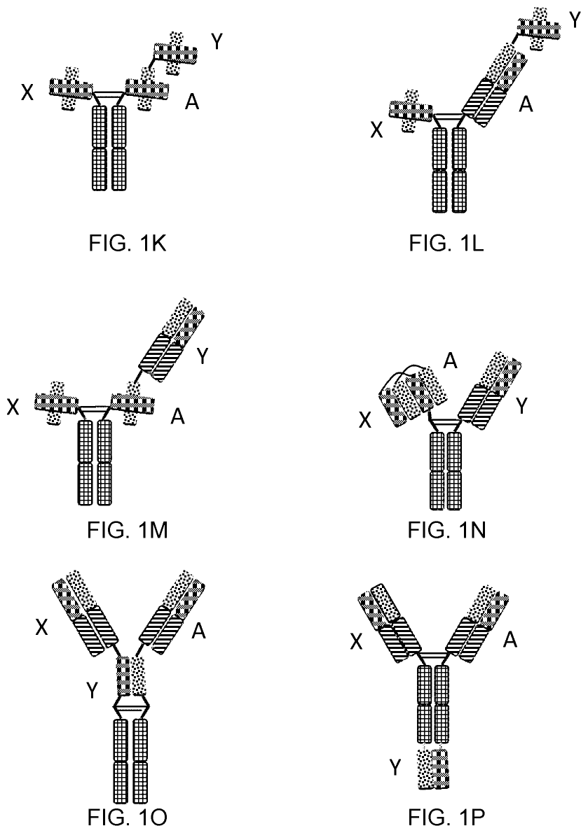

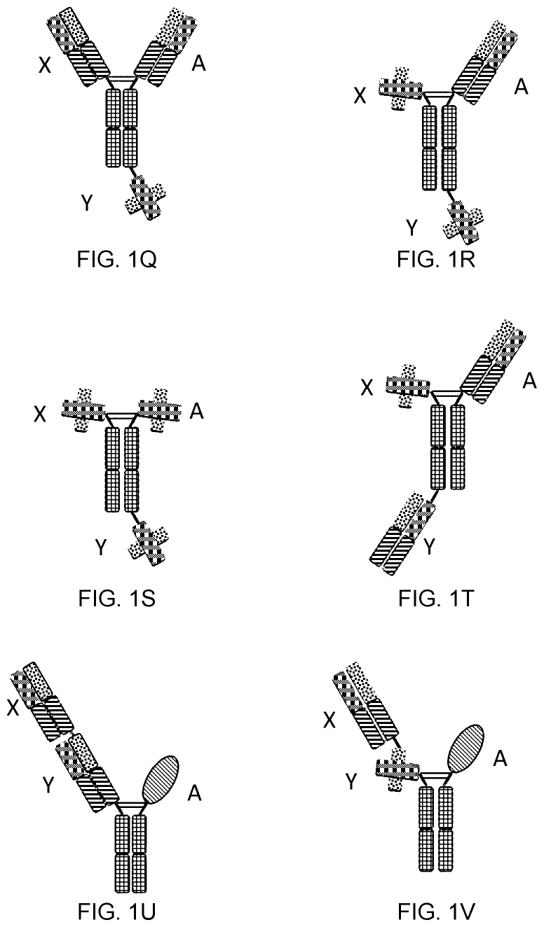

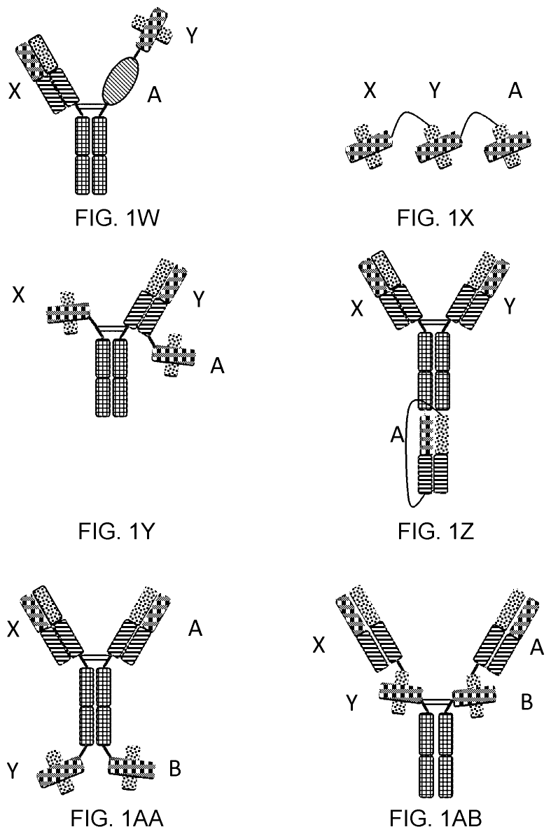

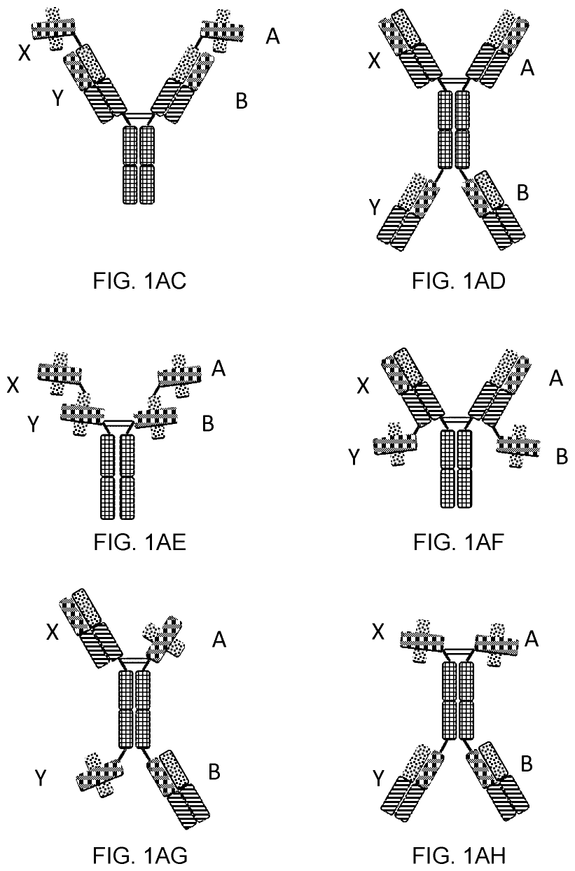

Non-immunoglobulin scaffolds that can be used in the MBMs include those listed in Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18. The contents of Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18 (collectively, “Scaffold Disclosures”) are incorporated by reference herein. In a particular embodiment, the Scaffold Disclosures are incorporated by reference for what they disclose relating to Adnexins. In another embodiment, the Scaffold Disclosures are incorporated by reference for what they disclose relating to Avimers. In another embodiment, the Scaffold Disclosures are incorporated by reference for what they disclose relating to Affibodies. In yet another embodiment, the Scaffold Disclosures are incorporated by reference for what they disclose relating to Anticalins. In yet another embodiment, the Scaffold Disclosures are incorporated by reference for what they disclose relating to DARPins. In yet another embodiment, the Scaffold Disclosures are incorporated by reference for what they disclose relating to Kunitz domains. In yet another embodiment, the Scaffold Disclosures are incorporated by reference for what they disclose relating to Knottins. In yet another embodiment, the Scaffold Disclosures are incorporated by reference for what they disclose relating to Pronectins. In yet another embodiment, the Scaffold Disclosures are incorporated by reference for what they disclose relating to Nanofitins. In yet another embodiment, the Scaffold Disclosures are incorporated by reference for what they disclose relating to Affilins. In yet another embodiment, the Scaffold Disclosures are incorporated by reference for what they disclose relating to Adnectins. In yet another embodiment, the Scaffold Disclosures are incorporated by reference for what they disclose relating to ABMs. In yet another embodiment, the Scaffold Disclosures are incorporated by reference for what they disclose relating to Adhirons. In yet another embodiment, the Scaffold Disclosures are incorporated by reference for what they disclose relating to Affimers. In yet another embodiment, the Scaffold Disclosures are incorporated by reference for what they disclose relating to Alphabodies. In yet another embodiment, the Scaffold Disclosures are incorporated by reference for what they disclose relating to Armadillo Repeat Proteins. In yet another embodiment, the Scaffold Disclosures are incorporated by reference for what they disclose relating to Atrimers/Tetranectins. In yet another embodiment, the Scaffold Disclosures are incorporated by reference for what they disclose relating to Obodies/OB-folds. In yet another embodiment, the Scaffold Disclosures are incorporated by reference for what they disclose relating to Centyrins. In yet another embodiment, the Scaffold Disclosures are incorporated by reference for what they disclose relating to Repebodies. In yet another embodiment, the Scaffold Disclosures are incorporated by reference for what they disclose relating to Anticalins. In yet another embodiment, the Scaffold Disclosures are incorporated by reference for what they disclose relating to Atrimers. In yet another embodiment, the Scaffold Disclosures are incorporated by reference for what they disclose relating to bicyclic peptides. In yet another embodiment, the Scaffold Disclosures are incorporated by reference for what they disclose relating to cys-knots. In yet another embodiment, the Scaffold Disclosures are incorporated by reference for what they disclose relating to Fn3 scaffolds (including Adnectins, Centryrins, Pronectins, and Tn3).

In an embodiment, an ABM can be a designed ankyrin repeat protein (“DARPin”). DARPins are antibody mimetic proteins that typically exhibit highly specific and high-affinity target protein binding. They are typically genetically engineered and derived from natural ankyrin proteins and consist of at least three, usually four or five repeat motifs of these proteins. Their molecular mass is about 14 or 18 kDa (kilodaltons) for four- or five-repeat DARPins, respectively. Examples of DARPins can be found, for example in U.S. Pat. No. 7,417,130. Multispecific binding molecules comprising DARPin binding modules and immunoglobulin-based binding modules are disclosed in, for example, U.S. Publication No. 2015/0030596 A1.

In another embodiment, an ABM can be an Affibody. An Affibody is well known in the art and refers to affinity proteins based on a 58 amino acid residue protein domain, derived from one of the IgG binding domain of staphylococcal protein A.

In another embodiment, an ABM can be an Anticalin. Anticalins are well known in the art and refer to another antibody mimetic technology, wherein the binding specificity is derived from Lipocalins. Anticalins can also be formatted as dual targeting protein, called Duocalins.

In another embodiment, an ABM can be a Versabody. Versabodies are well known in the art and refer to another antibody mimetic technology. They are small proteins of 3-5 kDa with >15% cysteines, which form a high disulfide density scaffold, replacing the hydrophobic core the typical proteins have.

Other non-immunoglobulin ABMs include “A” domain oligomers (also known as Avimers) (see for example, U.S. Patent Application Publication Nos. 2005/0164301, 2005/0048512, and 2004/017576), Fn3 based protein scaffolds (see for example, U.S. Patent Application Publication 2003/0170753), VASP polypeptides, Avian pancreatic polypeptide (aPP), Tetranectin (based on CTLD3), Affililin (based on γB-crystallin/ubiquitin), Knottins, SH3 domains, PDZ domains, Tendamistat, Neocarzinostatin, Protein A domains, Lipocalins, Transferrin, and Kunitz domains. In one aspect, ABMs useful in the construction of the MBMs comprise fibronectin-based scaffolds as exemplified in WO 2011/130324.

Moreover, in certain aspects, an ABM comprises a ligand binding domain of a receptor or a receptor binding domain of a ligand. For example, if the TAA is the EGF receptor, ABM3 can comprise a portion of EGF that binds EGFR, and if the TAA is the PDGF receptor, ABM3 can comprise a portion of PDGF receptor that binds PDGF, and so forth. In a specific embodiment, ABM1 is a CD2 ligand, in particular a CD58 moiety as described in Section 7.9.2. The respective binding domains of numerous ligand/receptor pairs are well known in the art, and thus can be readily selected and adapted for use in the MBMs.

### 7.4. Connectors

It is contemplated that the CD3 binding molecules (e.g., MBMs) can in some instances include pairs of ABMs or ABM chains (e.g., the VH-CH1 or VL-CL component of a Fab) connected directly to one another, e.g., as a fusion protein without a linker. For example, the CD3 binding molecules (e.g., MBMs) comprise connector moieties linking individual ABMs or ABM chains. The use of connector moieties can improve target binding, for example by increasing flexibility of the ABMs within a CD3 binding molecule (e.g., MBM) and thus reducing steric hindrance. The ABMs can be connected to one another through, for example, Fc domains (each Fc domain representing a pair of associated Fc regions) and/or ABM linkers. The use of Fc domains will typically require the use of hinge regions as connectors of the ABMs or ABM chains for optimal antigen binding. Thus, the term “connector” encompasses, but is not limited to, Fc regions, Fc domains, hinge regions, and ABM linkers.

Examples of Fc domains (formed by the pairing of two Fc regions), hinge regions and ABM linkers are described in Sections 7.4.1, 7.4.2, and 7.4.3, respectively.

7.4.1. Fc Domains

The CD3 binding molecules (e.g., MBMs) can include an Fc domain derived from any suitable species. In one embodiment, the Fc domain is derived from a human Fc domain.

The Fc domain can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment, the Fc domain is derived from IgG1. In one embodiment, the Fc domain is derived from IgG4.

The Fc domain comprises two polypeptide chains, each referred to as a heavy chain Fc region. The two heavy chain Fc regions dimerize to create the Fc domain. The two Fc regions within the Fc domain can be the same or different from one another. In a native antibody, the Fc regions are typically identical, but for the purpose of producing multispecific binding molecules, e.g., the MBMs, the Fc regions might advantageously be different to allow for heterodimerization, as described in Section 7.4.1.5 below.

Typically, each heavy chain Fc region comprises or consists of two or three heavy chain constant domains.

In native antibodies, the heavy chain Fc region of IgA, IgD and IgG is composed of two heavy chain constant domains (CH2 and CH3) and that of IgE and IgM is composed of three heavy chain constant domains (CH2, CH3 and CH4). These dimerize to create an Fc domain.

In the present disclosure, the heavy chain Fc region can comprise heavy chain constant domains from one or more different classes of antibody, for example one, two or three different classes.

In one embodiment, the heavy chain Fc region comprises CH2 and CH3 domains derived from IgG1.

In one embodiment, the heavy chain Fc region comprises CH2 and CH3 domains derived from IgG2.

In one embodiment, the heavy chain Fc region comprises CH2 and CH3 domains derived from IgG3.

In one embodiment, the heavy chain Fc region comprises CH2 and CH3 domains derived from IgG4.

In one embodiment, the heavy chain Fc region comprises a CH4 domain from IgM. The IgM CH4 domain is typically located at the C-terminus of the CH3 domain.

In one embodiment, the heavy chain Fc region comprises CH2 and CH3 domains derived from IgG and a CH4 domain derived from IgM.

It will be appreciated that the heavy chain constant domains for use in producing a heavy chain Fc region for the CD3 binding molecules (e.g., MBMs) of the present disclosure can include variants of the naturally occurring constant domains described above. Such variants can comprise one or more amino acid variations compared to wild type constant domains. In one example, the heavy chain Fc region of the present disclosure comprises at least one constant domain that varies in sequence from the wild type constant domain. It will be appreciated that the variant constant domains can be longer or shorter than the wild type constant domain. Preferably, the variant constant domains are at least 60% identical or similar to a wild type constant domain. In another example, the variant constant domains are at least 70% identical or similar. In another example, the variant constant domains are at least 80% identical or similar. In another example, the variant constant domains are at least 90% identical or similar. In another example, the variant constant domains are at least 95% identical or similar. Exemplary Fc variants are described in Sections 7.4.1.1 through 7.4.1.5.

IgM and IgA occur naturally in humans as covalent multimers of the common H2L2 antibody unit. IgM occurs as a pentamer when it has incorporated a J-chain or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the CD3 binding molecules (e.g., MBMs) of the present disclosure do not comprise a tailpiece.

The Fc domains that are incorporated into the CD3 binding molecules (e.g., MBMs) of the present disclosure can comprise one or more modifications that alter the functional properties of the proteins, such as serum half-life, complement fixation, Fc receptor binding, and/or antigen-dependent cellular cytotoxicity. Furthermore, a CD3 binding molecule can be chemically modified (e.g., one or more chemical moieties can be attached to the CD3 binding molecule) or be modified to alter its glycosylation, again to alter one or more functional properties of the CD3 binding molecule.

Effector function of an antibody molecule includes complement-mediated effector function, which is mediated by, for example, binding of the C1 component of the complement to the antibody. Activation of complement is important in the opsonization and direct lysis of pathogens. In addition, it stimulates the inflammatory response by recruiting and activating phagocytes to the site of complement activation. Effector function includes Fc receptor (FcR)-mediated effector function, which can be triggered upon binding of the constant domains of an antibody to an Fc receptor (FcR). Antigen-antibody complex-mediated crosslinking of Fc receptors on effector cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Fc regions can be altered by replacing at least one amino acid residue with a different amino acid residue to alter the effector functions. For example, one or more amino acids can be replaced with a different amino acid residue such that the Fc region has an altered affinity for an effector ligand. The effector ligand to which affinity is altered can be, for example, an Fc receptor or the C1 component of complement. This approach is described in, e.g., U.S. Pat. Nos. 5,624,821 and 5,648,260, both by Winter et al. Modified Fc regions can also alter C1q binding and/or reduce or abolish complement dependent cytotoxicity (CDC). This approach is described in, e.g., U.S. Pat. No. 6,194,551 by Idusogie et al. Modified Fc regions can also alter the ability of an Fc region to fix complement. This approach is described in, e.g., the PCT Publication WO 94/29351 by Bodmer et al. Allotypic amino acid residues include, but are not limited to, constant region of a heavy chain of the IgG1, IgG2, and IgG3 subclasses as well as constant region of a light chain of the kappa isotype as described by Jefferis et al., 2009, MAbs, 1:332-338.

Fc regions can also be modified to “silence” the effector function, for example, to reduce or eliminate the ability of a CD3 binding molecule to mediate antibody dependent cellular cytotoxicity (ADCC) and/or antibody dependent cellular phagocytosis (ADCP). This can be achieved, for example, by introducing a mutation in an Fc region. Such mutations have been described in the art: LALA and N297A (Strohl, 2009, Curr. Opin. Biotechnol. 20(6):685-691); and D265A (Baudino et al., 2008, J. Immunol. 181: 6664-69; Strohl, supra). Examples of silent Fc IgG1 antibodies comprise the so-called LALA mutant comprising L234A and L235A mutation in the IgG1 Fc amino acid sequence. Another example of a silent IgG1 antibody comprises the D265A mutation. Another silent IgG1 antibody comprises the so-called DAPA mutant comprising D265A and P329A mutations in the IgG1 Fc amino acid sequence. Another silent IgG1 antibody comprises the N297A mutation, which results in aglycosylated/non-glycosylated antibodies.

Fc regions can be modified to increase the ability of a CD3 binding molecule containing the Fc region to mediate antibody dependent cellular cytotoxicity (ADCC) and/or antibody dependent cellular phagocytosis (ADCP), for example, by modifying one or more amino acid residues to increase the affinity of the CD3 binding molecule for an activating Fcγ receptor, or to decrease the affinity of the CD3 binding molecule for an inhibitory Fcγ receptor. Human activating Fcγ receptors include FcγRIa, FcγRIIa, FcγRIIIa, and FcγRIIIb, and human inhibitory Fcγ receptor includes FcγRIIb. This approach is described in, e.g., the PCT Publication WO 00/42072 by Presta. Moreover, binding sites on human IgG1 for FcγRI, FcγRII, FcγRIII and FcRn have been mapped and variants with improved binding have been described (see Shields et al., J. Biol. Chem. 276:6591-6604, 2001). Optimization of Fc-mediated effector functions of monoclonal antibodies such as increased ADCC/ADCP function has been described (see Strohl, 2009, Current Opinion in Biotechnology 20:685-691). Mutations that can enhance ADCC/ADCP function include one or more mutations selected from G236A, S239D, F243L, P247I, D280H, K290S, R292P, S298A, S298D, S298V, Y300L, V305I, A330L, I332E, E333A, K334A, A339D, A339Q, A339T, and P396L (all positions by EU numbering).

Fc regions can also be modified to increase the ability of a CD3 binding molecule to mediate ADCC and/or ADCP, for example, by modifying one or more amino acids to increase the affinity of the CD3 binding molecule for an activating receptor that would typically not recognize the parent CD3 binding molecule, such as FcαRI. This approach is described in, e.g., Borrok et al., 2015, mAbs. 7(4):743-751.

Accordingly, in certain aspects, the CD3 binding molecules of the present disclosure can include Fc domains with altered effector function such as, but not limited to, binding to Fc-receptors such as FcRn or leukocyte receptors (for example, as described in Section 7.4.1.1), binding to complement (for example as described in Section 7.4.1.2), modified disulfide bond architecture (for example as described in Section 7.4.1.3), or altered glycosylation patterns (for example as described in Section 7.4.1.4). The Fc domains can also be altered to include modifications that improve manufacturability of asymmetric CD3 binding molecules (e.g., MBMs), for example by allowing heterodimerization, which is the preferential pairing of non-identical Fc regions over identical Fc regions. Heterodimerization permits the production of CD3 binding molecules (e.g., MBMs) in which different ABMs are connected to one another by an Fc domain containing Fc regions that differ in sequence. Examples of heterodimerization strategies are exemplified in Section 7.4.1.5 (and subsections thereof).

It will be appreciated that any of the modifications described in Sections 7.4.1.1 through 7.4.1.5 can be combined in any suitable manner to achieve the desired functional properties and/or combined with other modifications to alter the properties of the CD3 binding molecules (e.g., MBMs).

7.4.1.1. Fc Domains with Altered FcR Binding

The Fc domains of the CD3 binding molecules (e.g., MBMs) can show altered binding to one or more Fc-receptors (FcRs) in comparison with the corresponding native immunoglobulin. The binding to any particular Fc-receptor can be increased or decreased. In one embodiment, the Fc domain comprises one or more modifications which alter its Fc-receptor binding profile.

Human cells can express a number of membrane bound FcRs selected from FcαR, FcεR, FcγR, FcRn and glycan receptors. Some cells are also capable of expressing soluble (ectodomain) FcR (Fridman et al., 1993, J Leukocyte Biology 54: 504-512 for review). FcγR can be further divided by affinity of IgG binding (high/low) and biological effect (activating/inhibiting). Human FcγRI is widely considered the sole ‘high affinity’ receptor whilst all of the others are considered as medium to low. FcγRIIb is the sole receptor with ‘inhibitory’ functionality by virtue of its intracellular ITIM motif whilst all of the others are considered as ‘activating’ by virtue of ITAM motifs or pairing with the common FcγR-γchain. FcγRIIIb is also unique in that although activatory it associates with the cell via a GPI anchor. In total, humans express six “standard” FcγRs: FcγRI, FcγRIIa, FcγRIIb, FcγRIIc, FcγRIIIa FcγRIIIb. In addition to these sequences, there are a large number of sequence or allotypic variants spread across these families. Some of these have been found to have important functional consequence and so are sometimes considered receptor sub-types of their own. Examples include FcγRIIa^{H134R}, FcγRIIb^{I190T}, FcγRIIIa^{F158V} and FcγRIIIb^{NA1}, FcγRIIIb^{NA2} FcγRIII^{SH}. Each receptor sequence has been shown to have different affinities for the four sub-classes of IgG: IgG1, IgG2, IgG3 and IgG4 (Bruhns, 1993, Blood 113:3716-3725). Other species have somewhat different numbers and functionality of FcγR, with the mouse system being the best studied to date and comprising of four FcγR, FcγRI FcγRIIb FcγRIII FcγRIV (Bruhns, 2012, Blood 119:5640-5649). Human FcγRI on cells is normally considered to be ‘occupied’ by monomeric IgG in normal serum conditions due to its affinity for IgG1/IgG3/IgG4 (about 10⁻⁸ M) and the concentration of these IgG in serum (about 10 mg/ml). Hence, cells bearing FcγRI on their surface are considered capable for “screening” or “sampling” of their antigenic environment vicariously through the bound polyspecific IgG. The other receptors having lower affinities for IgG sub-classes (in the range of about 10⁻⁵-10⁻⁷ M) are normally considered to be “unoccupied.” The low affinity receptors are hence inherently sensitive to the detection of and activation by antibody involved immune complexes. The increased Fc density in an antibody immune complex results in increased functional affinity of binding avidity to low affinity FcγR. This has been demonstrated in vitro using a number of methods (Shields et al., 2001, J Biol Chem 276(9):6591-6604; Lux et al., 2013, J Immunol 190:4315-4323). It has also been implicated as being one of the primary modes of action in the use of anti-RhD to treat ITP in humans (Crow, 2008, Transfusion Medicine Reviews 22:103-116).

Many cell types express multiple types of FcγR and so binding of IgG or antibody immune complex to cells bearing FcγR can have multiple and complex outcomes depending upon the biological context. Most simply, cells can either receive an activatory, inhibitory or mixed signal. This can result in events such as phagocytosis (e.g., macrophages and neutrophils), antigen processing (e.g., dendritic cells), reduced IgG production (e.g., B-cells) or degranulation (e.g., neutrophils, mast cells). There are data to support that the inhibitory signal from FcγRIIb can dominate that of activatory signals (Proulx, 2010, Clinical Immunology 135:422-429).

FcRn has a crucial role in maintaining the long half-life of IgG in the serum of adults and children. The receptor binds IgG in acidified vesicles (pH<6.5) protecting the IgG molecule from degradation, and then releasing it at the higher pH of 7.4 in blood.

FcRn is unlike leukocyte Fc receptors, and instead, has structural similarity to MHC class I molecules. It is a heterodimer composed of a β₂-microglobulin chain, non-covalently attached to a membrane-bound chain that includes three extracellular domains. One of these domains, including a carbohydrate chain, together with β₂-microglobulin interacts with a site between the CH2 and CH3 domains of Fc. The interaction includes salt bridges made to histidine residues on IgG that are positively charged at pH<6.5. At higher pH, the His residues lose their positive charges, the FcRn-IgG interaction is weakened and IgG dissociates.

In one embodiment, a CD3 binding molecule (e.g., MBM) comprises an Fc domain that binds to human FcRn.

In one embodiment, the Fc domain has an (e.g., one or two) Fc regions comprising a histidine residue at position 310, and preferably also at position 435. These histidine residues are important for human FcRn binding. In one embodiment, the histidine residues at positions 310 and 435 are native residues, i.e., positions 310 and 435 are not modified. Alternatively, one or both of these histidine residues can be present as a result of a modification.

The CD3 binding molecules (e.g., MBMs) can comprise one or more Fc regions that alter Fc binding to FcRn. The altered binding can be increased binding or decreased binding.

In one embodiment, the CD3 binding molecule (e.g., MBM) comprises an Fc domain in which at least one (and optionally both) Fc regions comprises one or more modifications such that it binds to FcRn with greater affinity and avidity than the corresponding native immunoglobulin.

In one embodiment, the Fc region is modified by substituting the threonine residue at position 250 with a glutamine residue (T250Q).

In one embodiment, the Fc region is modified by substituting the methionine residue at position 252 with a tyrosine residue (M252Y)

In one embodiment, the Fc region is modified by substituting the serine residue at position 254 with a threonine residue (S254T).

In one embodiment, the Fc region is modified by substituting the threonine residue at position 256 with a glutamic acid residue (T256E).

In one embodiment, the Fc region is modified by substituting the threonine residue at position 307 with an alanine residue (T307A).

In one embodiment, the Fc region is modified by substituting the threonine residue at position 307 with a proline residue (T307P).

In one embodiment, the Fc region is modified by substituting the valine residue at position 308 with a cysteine residue (V308C).

In one embodiment, the Fc region is modified by substituting the valine residue at position 308 with a phenylalanine residue (V308F).

In one embodiment, the Fc region is modified by substituting the valine residue at position 308 with a proline residue (V308P).

In one embodiment, the Fc region is modified by substituting the glutamine residue at position 311 with an alanine residue (Q311A).

In one embodiment, the Fc region is modified by substituting the glutamine residue at position 311 with an arginine residue (Q311R).

In one embodiment, the Fc region is modified by substituting the methionine residue at position 428 with a leucine residue (M428L).

In one embodiment, the Fc region is modified by substituting the histidine residue at position 433 with a lysine residue (H433K).

In one embodiment, the Fc region is modified by substituting the asparagine residue at position 434 with a phenylalanine residue (N434F).

In one embodiment, the Fc region is modified by substituting the asparagine residue at position 434 with a tyrosine residue (N434Y).

In one embodiment, the Fc region is modified by substituting the methionine residue at position 252 with a tyrosine residue, the serine residue at position 254 with a threonine residue, and the threonine residue at position 256 with a glutamic acid residue (M252Y/S254T/T256E).

In one embodiment, the Fc region is modified by substituting the valine residue at position 308 with a proline residue and the asparagine residue at position 434 with a tyrosine residue (V308P/N434Y).

In one embodiment, the Fc region is modified by substituting the methionine residue at position 252 with a tyrosine residue, the serine residue at position 254 with a threonine residue, the threonine residue at position 256 with a glutamic acid residue, the histidine residue at position 433 with a lysine residue and the asparagine residue at position 434 with a phenylalanine residue (M252Y/S254T/T256E/H433K/N434F).

It will be appreciated that any of the modifications listed above can be combined to alter FcRn binding.

In one embodiment, the CD3 binding molecule (e.g., MBM) comprises an Fc domain in which one or both Fc regions comprise one or more modifications such that the Fc domain binds to FcRn with lower affinity and avidity than the corresponding native immunoglobulin.

In one embodiment, the Fc region comprises any amino acid residue other than histidine at position 310 and/or position 435.

The CD3 binding molecule (e.g., MBM) can comprise an Fc domain in which one or both Fc regions comprise one or more modifications, which increase its binding to FcγRIIb. FcγRIIb is the only inhibitory receptor in humans and the only Fc receptor found on B cells.

In one embodiment, the Fc region is modified by substituting the proline residue at position 238 with an aspartic acid residue (P238D).

In one embodiment, the Fc region is modified by substituting the glutamic acid residue at position 258 with an alanine residue (E258A).

In one embodiment, the Fc region is modified by substituting the serine residue at position 267 with an alanine residue (S267A).

In one embodiment, the Fc region is modified by substituting the serine residue at position 267 with a glutamic acid residue (S267E).

In one embodiment, the Fc region is modified by substituting the leucine residue at position 328 with a phenylalanine residue (L328F).

In one embodiment, the Fc region is modified by substituting the glutamic acid residue at position 258 with an alanine residue and the serine residue at position 267 with an alanine residue (E258A/S267A).

In one embodiment, the Fc region is modified by substituting the serine residue at position 267 with a glutamic acid residue and the leucine residue at position 328 with a phenylalanine residue (S267E/L328F).

It will be appreciated that any of the modifications listed above can be combined to increase FcγRIIb binding.

In one embodiment, CD3 binding molecules (e.g., MBMs) are provided comprising Fc domains which display decreased binding to FcγR.

In one embodiment, a CD3 binding molecule (e.g., MBM) comprises an Fc domain in which one or both Fc regions comprise one or more modifications that decrease Fc binding to FcγR.

The Fc domain can be derived from IgG1.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the glycine residue at position 236 with an arginine residue (G236R).

In one embodiment, the Fc region is modified by substituting the asparagine residue at position 297 with an alanine residue (N297A) or a glutamine residue (N297Q).

In one embodiment, the Fc region is modified by substituting the serine residue at position 298 with an alanine residue (S298A).

In one embodiment, the Fc region is modified by substituting the leucine residue at position 328 with an arginine residue (L328R).

In one embodiment, the Fc region is modified by substituting the leucine residue at position 234 with an alanine residue and the leucine residue at position 235 with an alanine residue (L234A/L235A).

In one embodiment, the Fc region is modified by substituting the phenylalanine residue at position 234 with an alanine residue and the leucine residue at position 235 with an alanine residue (F234A/L235A).

In one embodiment, the Fc region is modified by substituting the glycine residue at position 236 with an arginine residue and the leucine residue at position 328 with an arginine residue (G236R/L328R).

It will be appreciated that any of the modifications listed above can be combined to decrease FcγR binding.

In one embodiment, a CD3 binding molecule (e.g., MBM) of the present disclosure comprises an Fc domain in which one or both Fc regions comprise one or more modifications that decrease Fc binding to FcγRIIIa without affecting the Fc's binding to FcγRII.

In one embodiment, the Fc region is modified by substituting the serine residue at position 239 with an alanine residue (S239A).

In one embodiment, the Fc region is modified by substituting the glutamic acid residue at position 269 with an alanine residue (E269A).

In one embodiment, the Fc region is modified by substituting the glutamic acid residue at position 293 with an alanine residue (E293A).

In one embodiment, the Fc region is modified by substituting the tyrosine residue at position 296 with a phenylalanine residue (Y296F).

In one embodiment, the Fc region is modified by substituting the valine residue at position 303 with an alanine residue (V303A).

In one embodiment, the Fc region is modified by substituting the alanine residue at position 327 with a glycine residue (A327G).

In one embodiment, the Fc region is modified by substituting the lysine residue at position 338 with an alanine residue (K338A).

In one embodiment, the Fc region is modified by substituting the aspartic acid residue at position 376 with an alanine residue (D376A).

It will be appreciated that any of the modifications listed above can be combined to decrease FcγRIIIa binding.

Fc region variants with decreased FcR binding can be referred to as “FcγR ablation variants,” “FcγR silencing variants” or “Fc knock out (FcKO or KO)” variants. For some therapeutic applications, it is desirable to reduce or remove the normal binding of an Fc domain to one or more or all of the Fcγ receptors (e.g., FcγR1, FcγRIIa, FcγRIIb, FcγRIIIa) to avoid additional mechanisms of action. That is, for example, in many embodiments, particularly in the use of MBMs that bind CD3 monovalently, it is generally desirable to ablate FcγRIIIa binding to eliminate or significantly reduce ADCC activity. In some embodiments, at least one of the Fc regions of the MBMs described herein comprises one or more Fcγ receptor ablation variants. In some embodiments, both of the Fc regions comprise one or more Fcγ receptor ablation variants. These ablation variants are depicted in Table 3, and each can be independently and optionally included or excluded, with some aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del (“del” connotes a deletion, e.g., G236del refers to a deletion of the glycine at position 236). It should be noted that the ablation variants referenced herein ablate FcγR binding but generally not FcRn binding.

TABLE 3
Ablation Variants

| Variant | Variant(s), cont. |
|---|---|
| G236R | P329K |
| S239G | A330L |
| S239K | A330S/P331S |
| S239Q | I332K |
| S239R | I332R |
| V266D | V266D/A327Q |
| S267K | V266D/P329K |
| S267R | S267R/A327Q |
| H268K | S267R/P329K |
| E269R | G236R/L328R |
| 299R | E233P/L234V/L235A/G236del/S239K |
| 299K | E233P/L234V/L235A/G236del/S267K |
| K322A | E233P/L234V/L235A/G236del/S239K/A327G |
| A327G | E233P/L234V/L235A/G236del/S267K/A327G |
| A327L | E233P/L234V/L235A/G236del |
| A327N | S239K/S267K |
| A327Q | 267K/P329K |
| L328E | |
| L328R | |
| P329A | |
| P329H | |

In some embodiments, a CD3 binding molecule (e.g., MBM) of the present disclosure comprises a first Fc region and a second Fc region. In some embodiments, the first Fc region and/or the second Fc region can comprise the following mutations: E233P, L234V, L235A, G236del, and S267K.

The Fc domain of human IgG1 has the highest binding to the Fcγ receptors, and thus ablation variants can be used when the constant domain (or Fc domain) in the backbone of the heterodimeric antibody is IgG1.

Alternatively, or in addition to ablation variants in an IgG1 background, mutations at the glycosylation position 297, e.g., substituting the asparagine residue at position 297 with an alanine residue (N297A) or a glutamine residue (N297Q), can significantly ablate binding to FcγRIIIa, for example. Human IgG2 and IgG4 have naturally reduced binding to the Fcγ receptors, and thus those backbones can be used with or without the ablation variants.

7.4.1.2. Fc Domains with Altered Complement Binding

The CD3 binding molecule (e.g., MBM) can comprise an Fc domain in which one or both Fc regions comprises one or more modifications that alter Fc binding to complement. Altered complement binding can be increased binding or decreased binding.

In one embodiment, the Fc region comprises one or more modifications, which decrease its binding to C1q. Initiation of the classical complement pathway starts with binding of hexameric C1q protein to the CH2 domain of antigen bound IgG and IgM.

In one embodiment, the CD3 binding molecule (e.g., MBM) comprises an Fc domain in which one or both Fc regions comprises one or more modifications to decrease Fc binding to C1q.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the leucine residue at position 235 with a glutamic acid residue (L235E).

In one embodiment, the Fc region is modified by substituting the glycine residue at position 237 with an alanine residue (G237A).

In one embodiment, the Fc region is modified by substituting the lysine residue at position 322 with an alanine residue (K322A).

In one embodiment, the Fc region is modified by substituting the proline residue at position 331 with an alanine residue (P331A).

In one embodiment, the Fc region is modified by substituting the proline residue at position 331 with a serine residue (P331S).

In one embodiment, a CD3 binding molecule (e.g., MBM) comprises an Fc domain derived from IgG4. IgG4 has a naturally lower complement activation profile than IgG1, but also weaker binding of FcγR. Thus, in one embodiment, the CD3 binding molecule (e.g., MBM) comprises an IgG4 Fc domain and comprises one or more modifications that increase FcγR binding.

It will be appreciated that any of the modifications listed above can be combined to reduce C1q binding.

7.4.1.3. Fc Domains with Altered Disulfide Architecture

The CD3 binding molecules (e.g., MBMs) can include an Fc domain comprising one or more modifications to create and/or remove a cysteine residue. Cysteine residues have an important role in the spontaneous assembly of Fc-based multispecific binding molecules, by forming disulfide bridges between individual pairs of polypeptide monomers. Thus, by altering the number and/or position of cysteine residues, it is possible to modify the structure of the CD3 binding molecule (e.g., MBM) to produce a protein with improved therapeutic properties.

A CD3 binding molecule (e.g., MBM) of the present disclosure can comprise an Fc domain in which one or both Fc regions, preferably both Fc regions, comprise a cysteine residue at position 309. In one embodiment, the cysteine residue at position 309 is created by a modification, e.g., for an Fc domain derived from IgG1, the leucine residue at position 309 is substituted with a cysteine residue (L309C), for an Fc domain derived from IgG2, the valine residue at position 309 is substituted with a cysteine residue (V309C).

In one embodiment, the Fc region is modified by substituting the valine residue at position 308 with a cysteine residue (V308C).

In one embodiment, two disulfide bonds in the hinge region are removed by mutating a core hinge sequence CPPC (SEQ ID NO: 9) to SPPS (SEQ ID NO: 14).

7.4.1.4. Fc Domains with Altered Glycosylation

In certain aspects, CD3 binding molecules (e.g., MBMs) with improved manufacturability are provided that comprise fewer glycosylation sites than a corresponding immunoglobulin. These proteins have less complex post translational glycosylation patterns and are thus simpler and less expensive to manufacture.

In one embodiment, a glycosylation site in the CH2 domain is removed by substituting the asparagine residue at position 297 with an alanine residue (N297A) or a glutamine residue (N297Q). In addition to improved manufacturability, these aglycosyl mutants also reduce FcγR binding as described herein above.

In some embodiments, a CD3 binding molecule can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing a CD3 binding molecule in a host cell with altered glycosylation machinery. Cells with altered glycosylation machinery have been described in the art and can be used as host cells in which to express CD3 binding molecules to thereby produce CD3 binding molecules with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields et al., 2002, J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

7.4.1.5. Fc Heterodimerization

Many multispecific molecule formats entail dimerization between two Fc regions that, unlike a native immunoglobulin, are operably linked to non-identical antigen-binding domains (or portions thereof, e.g., a VH or VH-CH1 of a Fab). Inadequate heterodimerization of two Fc regions to form an Fc domain has always been an obstacle for increasing the yield of desired multispecific molecules and represents challenges for purification. A variety of approaches available in the art can be used in for enhancing dimerization of Fc regions that might be present in the CD3 binding molecules (e.g., MBMs) of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO2009/089004A1.

The present disclosure provides CD3 binding molecules (e.g., MBMs) comprising Fc heterodimers, i.e., Fc domains comprising heterologous, non-identical Fc regions. Heterodimerization strategies are used to enhance dimerization of Fc regions operably linked to different ABMs (or portions thereof, e.g., a VH or VH-CH1 of a Fab) and reduce dimerization of Fc regions operably linked to the same ABM or portion thereof. Typically, each Fc region in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Typically, the MBMs comprise other antibody fragments in addition to CH3 domains, such as, CH1 domains, CH2 domains, hinge domain, VH domain(s), VL domain(s), CDR(s), and/or antigen-binding fragments described herein. In some embodiments, the two hetero-polypeptides are two heavy chains forming a bispecific or multispecific molecules. Heterodimerization of the two different heavy chains at CH3 domains give rise to the desired antibody or antibody-like molecule, while homodimerization of identical heavy chains will reduce yield of the desired antibody or molecule. In an exemplary embodiment, the two or more hetero-polypeptide chains comprise two chains comprising CH3 domains and forming the molecules of any of the multispecific molecule formats described above of the present disclosure. In an embodiment, the two hetero-polypeptide chains comprising CH3 domains comprise modifications that favor heterodimeric association of the polypeptides, relative to unmodified chains. Various examples of modification strategies are provided below in Table 4 and Sections 7.4.1.5.1 to 7.4.1.5.8.

TABLE 4
Fc Heterodimerization Strategies

| NO. | STRATEGY | CH3 DOMAIN 1 | CH3 DOMAIN 2 | REFERENCES |
|---|---|---|---|---|
| Fc 1 | knobs-into-holes | T366Y | Y407T | Ridgway et al., 1996, |
| | (Y-T) | | | Protein Eng 9: 617-21 |
| Fc 2 | knobs-into-holes | S354C, T366W | Y349C, T366S, | Atwell et al., 1997, J |
| | (CW-CSAV) | | L368A, Y407V | Mol Biol. 270(1): 26-35; |
| | | | | Merchant et al., 1998, |
| | | | | Nat Biotechnol 16: |
| | | | | 677-681 |
| Fc 3 | HA-TF | S364H, F405A | Y349T, T394F | Moore et al., 2011, |
| | | | | MAbs 3(6): 546-57 |
| Fc 4 | ZW1 (VYAV-VLLW) | T350V, L351Y, | T350V, T366L, | Von Kreudenstein et |
| | | F405A, Y407V | K392L, T394W | al., 2013, MAbs 5: |
| | | | | 646-54 |
| Fc 5 | CH3 charge pairs | K392D, K409D | E356K, D399K | Gunasekaran et al., |
| | (DD-KK) | | | 2010, J Biol Chem |
| | | | | 285: 19637-46 |
| Fc 6 | IgG1 hingE, CH3 | IgG1: D221E, | IgG1: D221R, | Strop et al., 2012, J Mol |
| | charge pairs (EEE- | P228E, L368E | P228R, K409R | Biol 420: 204-19 |
| | RRR) | | | |
| Fc 7 | IgG2 hingE, CH3 | IgG2: C223E, | IgG2: C223R, | Strop et al., 2012, J Mol |
| | charge pairs (EEE- | P228E, L368E | E225R, P228R, | Biol 420: 204-19 |
| | RRRR) | | K409R | |
| Fc 8 | EW-RVT | K360E, K409W, | Q347R, D399V, | Choi et al., 2013, Mol |
| | | | F405T | Cancer Ther 12: 2748-59 |
| Fc 9 | EW-RVTS-S | K360E, K409W, | Q347R, D399V, | Choi et al., 2015, Mol |
| | | Y349C | F405T, S354C | Immunol 65: 377-83 |
| Fc 10 | Biclonic | 366K (+351K) | 351D or E or D at | Geuijen et al., 2014, |
| | | | 349, 368, 349, or | Journal of Clinical |
| | | | 349 + 355 | Oncology 32: suppl: 560 |
| Fc 11 | DuoBody (L-R) | F405L | K409R | Labrijn et al., 2013, |
| | | | | Proc Natl Acad Sci |
| | | | | USA 110: 5145-50 |
| Fc 12 | SEEDbody | IgG/A chimera | IgG/A chimera | Davis et al., 2010, |
| | | | | Protein Eng Des Sel |
| | | | | 23: 195-202 |
| Fc 13 | BEAT | residues from | residues from TCRβ | Moretti et al., 2013, |
| | | TCRα interface | interface | BMC Proceedings |
| | | | | 7(Suppl 6): O9 |
| Fc 14 | 7.8.60 (DMA-RRVV) | K360D, D399M, | E345R, Q347R, | Leaver-Fey et al., |
| | | Y407A | T366V, K409V | Structure 24: 641-51 |
| Fc 15 | 20.8.34 (SYMV- | Y349S, K370Y, | E356G, E357D, | Leaver-Fey et al., |
| | GDQA) | T366M, K409V | S364Q, Y407A | Structure 24: 641-51 |
| Fc 16 | Skew variant 12757 | None | none | FIG. 34 of US |
| | | | | 2016/0355600 |
| Fc 17 | Skew variant 12758 | L368D, K370S | S364K | FIG. 34 of US |
| | | | | 2016/0355600 |
| Fc 18 | Skew variant 12759 | L368D, K370S | S364K, E357L | FIG. 34 of US |
| | | | | 2016/0355600 |
| Fc 19 | Skew variant 12760 | L368D, K370S | S364K, E357Q | FIG. 34 of US |
| | | | | 2016/0355600 |
| Fc 20 | Skew variant 12761 | T411E, K360E, | D401K | FIG. 34 of US |
| | | Q362E | | 2016/0355600 |
| Fc 21 | Skew variant 12496 | L368E, K370S | S364K | FIG. 34 of US |
| | | | | 2016/0355600 |
| Fc 22 | Skew variant 12511 | K370S | S364K | FIG. 34 of US |
| | | | | 2016/0355600 |
| Fc 23 | Skew variant 12840 | L368E, K370S | S364K, E357Q | FIG. 34 of US |
| | | | | 2016/0355600 |
| Fc 24 | Skew variant 12841 | K370S | S364K, E357Q | FIG. 34 of US |
| | | | | 2016/0355600 |
| Fc 25 | Skew variant 12894 | L368E, K370S | S364K | FIG. 34 of US |
| | | | | 2016/0355600 |
| Fc 26 | Skew variant 12895 | K370S | S364K | FIG. 34 of US |
| | | | | 2016/0355600 |
| Fc 27 | Skew variant 12896 | L368E, K370S | S364K, E357Q | FIG. 34 of US |
| | | | | 2016/0355600 |
| Fc 28 | Skew variant 12901 | K370S | S364K, E357Q | FIG. 34 of US |
| | | | | 2016/0355600 |
| Fc 29 | pI_ISO(−) | I199T, N203D, | | FIG. 31 of US |
| | | K274Q, R355Q, | | 2016/0355600 |
| | | N384S, K392N, | | |
| | | V397M, Q419E, | | |
| | | DEL447 | | |
| Fc 30 | pI_(−)_Isosteric_A | N208D, Q295E, | | FIG. 31 of US |
| | | N384D, Q418E, | | 2016/0355600 |
| | | N421D | | |
| Fc 31 | pI_(−)_isosteric_B | N208D, Q295E, | | FIG. 31 of US |
| | | Q418E, N421D | | 2016/0355600 |
| Fc 32 | pI_ISO(+RR) | Q196K, I199T, | | FIG. 31 of US |
| | | P217R, P228R, | | 2016/0355600 |
| | | N276K | | |
| Fc 33 | pI_ISO(+) | Q196K, I199T, | | FIG. 31 of US |
| | | N276K | | 2016/0355600 |
| Fc 34 | pI_(+) isosteric_A | E269Q, E272Q, | | FIG. 31 of US |
| | | E283Q, E357Q, | | 2016/0355600 |
| Fc 35 | pI_(+)_isosteric_B | E269Q, E272Q, | | FIG. 31 of US |
| | | E283Q | | 2016/0355600 |
| Fc 36 | PI_(+) | E269Q, E272Q | | FIG. 31 of US |
| | isosteric_E269Q, | | | 2016/0355600 |
| | E272Q | | | |
| Fc 37 | pI_(+)_isosteric_E269Q, | E269Q, E283Q | | FIG. 31 of US |
| | E283Q | | | 2016/0355600 |
| Fc 38 | PI_(+) | E272Q, E283Q | | FIG. 31 of US |
| | isosteric_E2720, | | | 2016/0355600 |
| | E283Q | | | |
| Fc 39 | pI_(+)_isosteric_E269Q | E269Q | | FIG. 31 of US |
| | | | | 2016/0355600 |
| Fc 40 | Heterodimerization | F405A | T394F | FIG. 30A of US |
| | | | | 2016/0355600 |
| Fc 41 | Heterodimerization | S364D | Y349K | FIG. 30A of US |
| | | | | 2016/0355600 |
| Fc 42 | Heterodimerization | S364E | L368K | FIG. 30A of US |
| | | | | 2016/0355600 |
| Fc 43 | Heterodimerization | S364E | Y349K | FIG. 30A of US |
| | | | | 2016/0355600 |
| Fc 44 | Heterodimerization | S364F | K370G | FIG. 30A of US |
| | | | | 2016/0355600 |
| Fc 45 | Heterodimerization | S364H | Y349K | FIG. 30A of US |
| | | | | 2016/0355600 |
| Fc 46 | Heterodimerization | S364H | Y349T | FIG. 30A of US |
| | | | | 2016/0355600 |
| Fc 47 | Heterodimerization | S364Y | K370G | FIG. 30A of US |
| | | | | 2016/0355600 |
| Fc 48 | Heterodimerization | T411K | K370E | FIG. 30A of US |
| | | | | 2016/0355600 |
| Fc 49 | Heterodimerization | V397S, F405A | T394F | FIG. 30A of US |
| | | | | 2016/0355600 |
| Fc 50 | Heterodimerization | K370R, T411K | K370E, T411E | FIG. 30A of US |
| | | | | 2016/0355600 |
| Fc 51 | Heterodimerization | L351E, S364D | Y349K, L351K | FIG. 30A of US |
| | | | | 2016/0355600 |
| Fc 52 | Heterodimerization | L351E, S364E | Y349K, L351K | FIG. 30A of US |
| | | | | 2016/0355600 |
| Fc 53 | Heterodimerization | L351E, T366D | L351K, T366K | FIG. 30A of US |
| | | | | 2016/0355600 |
| Fc 54 | Heterodimerization | P395T, V397S, | T394F | FIG. 30A of US |
| | | F405A | | 2016/0355600 |
| Fc 55 | Heterodimerization | S364D, K370G | S364Y, K370R | FIG. 30A of US |
| | | | | 2016/0355600 |
| Fc 56 | Heterodimerization | S364D, T394F | Y349K, F405A | FIG. 30A of US |
| | | | | 2016/0355600 |
| Fc 57 | Heterodimerization | S364E, F405A | Y349K, T394F | FIG. 30A of US |
| | | | | 2016/0355600 |
| Fc 58 | Heterodimerization | S364E, F405S | Y349K, T394Y | FIG. 30A of US |
| | | | | 2016/0355600 |
| Fc 59 | Heterodimerization | S364E, T411E | Y349K, D401K | FIG. 30A of US |
| | | | | 2016/0355600 |
| Fc 60 | Heterodimerization | S364H, D401K | Y349T, T411E | FIG. 30A of US |
| | | | | 2016/0355600 |
| Fc 61 | Heterodimerization | S364H, F405A | Y349T, T394F | FIG. 30A of US |
| | | | | 2016/0355600 |
| Fc 62 | Heterodimerization | S364H, T394F | Y349T, F405A | FIG. 30A of US |
| | | | | 2016/0355600 |
| Fc 63 | Heterodimerization | Y349C, S364E | Y349K, S354C | FIG. 30A of US |
| | | | | 2016/0355600 |
| Fc 64 | Heterodimerization | L351E, S364D, | Y349K, L351K, | FIG. 30A of US |
| | | F405A | T394F | 2016/0355600 |
| Fc 65 | Heterodimerization | L351K, S364H, | Y349T, L351E, | FIG. 30A of US |
| | | D401K | T411E | 2016/0355600 |
| Fc 66 | Heterodimerization | S364E, T411E, | Y349K, T394F, | FIG. 30A of US |
| | | F405A | D401K | 2016/0355600 |
| Fc 67 | Heterodimerization | S364H, D401K, | Y349T, T394F, | FIG. 30A of US |
| | | F405A | T411E | 2016/0355600 |
| Fc 68 | Heterodimerization | S364H, F405A, | Y349T, T394F, | FIG. 30A of US |
| | | T411E | D401K | 2016/0355600 |
| Fc 69 | Heterodimerization | T411E, K360E, | D401K | FIG. 30C of US |
| | | N390D | | 2016/0355600 |
| Fc 70 | Heterodimerization | T411E, Q362E, | D401K | FIG. 30C of US |
| | | N390D | | 2016/0355600 |
| Fc 71 | Heterodimerization | T411E, Q347R | D401K, K360D | FIG. 30C of US |
| | | | | 2016/0355600 |
| Fc 72 | Heterodimerization | T411E, Q347R | D401K, K360E | FIG. 30C of US |
| | | | | 2016/0355600 |
| Fc 73 | Heterodimerization | T411E, K360 | D401K, Q347K | FIG. 30C of US |
| | | | | 2016/0355600 |
| Fc 74 | Heterodimerization | T411E, K360D | D401K, Q347R | FIG. 30C of US |
| | | | | 2016/0355600 |
| Fc 75 | Heterodimerization | T411E, K360E | D401K, Q347K | FIG. 30C of US |
| | | | | 2016/0355600 |
| Fc 76 | Heterodimerization | T411E, K360E | D401K, Q347R | FIG. 30C of US |
| | | | | 2016/0355600 |
| Fc 77 | Heterodimerization | T411E, S364K | D401K, K370S | FIG. 30C of US |
| | | | | 2016/0355600 |
| Fc 78 | Heterodimerization | T411E, K370S | D401K, S364K | FIG. 30C of US |
| | | | | 2016/0355600 |
| Fc 79 | Heterodimerization | Q347E | E357Q | FIG. 30C of US |
| | | | | 2016/0355600 |
| Fc 80 | Heterodimerization | Q347E | E357Q, Q362K | FIG. 30C of US |
| | | | | 2016/0355600 |
| Fc 81 | Heterodimerization | K360D, Q362E | Q347R | FIG. 30C of US |
| | | | | 2016/0355600 |
| Fc 82 | Heterodimerization | K360D, Q362E | D401K | FIG. 30C of US |
| | | | | 2016/0355600 |
| Fc 83 | Heterodimerization | K360D, Q362E | Q347R, D401K | FIG. 30C of US |
| | | | | 2016/0355600 |
| Fc 84 | Heterodimerization | K360E, Q362E | Q347R | FIG. 30C of US |
| | | | | 2016/0355600 |
| Fc 85 | Heterodimerization | K360E, Q362E | D401K | FIG. 30C of US |
| | | | | 2016/0355600 |
| Fc 86 | Heterodimerization | K360E, Q362E | Q347R, D401K | FIG. 30C of US |
| | | | | 2016/0355600 |
| Fc 87 | Heterodimerization | Q362E, N390D | D401K | FIG. 30C of US |
| | | | | 2016/0355600 |
| Fc 88 | Heterodimerization | Q347E, K360D | D401N | FIG. 30C of US |
| | | | | 2016/0355600 |
| Fc 89 | Heterodimerization | K360D | Q347R, N390K | FIG. 30C of US |
| | | | | 2016/0355600 |
| Fc 90 | Heterodimerization | K360D | N390K, D401N | FIG. 30C of US |
| | | | | 2016/0355600 |
| Fc 91 | Heterodimerization | K360E | Y349H | FIG. 30C of US |
| | | | | 2016/0355600 |
| Fc 92 | Heterodimerization | K370S, Q347E | S364K | FIG. 30C of US |
| | | | | 2016/0355600 |
| Fc 93 | Heterodimerization | K370S, E357L | S364K | FIG. 30C of US |
| | | | | 2016/0355600 |
| Fc 94 | Heterodimerization | K370S, E357Q | S364K | FIG. 30C of US |
| | | | | 2016/0355600 |
| Fc 95 | Heterodimerization | K370S, Q347E, | S364K | FIG. 30C of US |
| | | E357L | | 2016/0355600 |
| Fc 96 | Heterodimerization | K370S, Q347E, | S364K | FIG. 30C of US |
| | | E357Q | | 2016/0355600 |
| Fc 97 | Heterodimerization | L368D, K370S, | S364K | FIG. 30D of US |
| | | Q347E | | 2016/0355600 |
| Fc 98 | Heterodimerization | L368D, K370S, | S364K | FIG. 30D of US |
| | | E357L | | 2016/0355600 |
| Fc 99 | Heterodimerization | L368D, K370S, | S364K | FIG. 30D of US |
| | | E357Q | | 2016/0355600 |
| Fc 100 | Heterodimerization | L368D, K370S, | S364K | FIG. 30D of US |
| | | Q347E, E357L | | 2016/0355600 |
| Fc 101 | Heterodimerization | L368D, K370S, | S364K | FIG. 30D of US |
| | | Q347E, E357Q | | 2016/0355600 |
| Fc 102 | Heterodimerization | L368E, K370S, | S364K | FIG. 30D of US |
| | | Q347E | | 2016/0355600 |
| Fc 103 | Heterodimerization | L368E, K370S, | S364K | FIG. 30D of US |
| | | E357L | | 2016/0355600 |
| Fc 104 | Heterodimerization | L368E, K370S, | S364K | FIG. 30D of US |
| | | E357Q | | 2016/0355600 |
| Fc 105 | Heterodimerization | L368E, K370S, | S364K | FIG. 30D of US |
| | | Q347E, E357L | | 2016/0355600 |
| Fc 106 | Heterodimerization | L368E, K370S, | S364K | FIG. 30D of US |
| | | Q347E, E357Q | | 2016/0355600 |
| Fc 107 | Heterodimerization | L368D, K370T, | S364K | FIG. 30D of US |
| | | Q347E | | 2016/0355600 |
| Fc 108 | Heterodimerization | L368D, K370T, | S364K | FIG. 30D of US |
| | | E357L | | 2016/0355600 |
| Fc 109 | Heterodimerization | L368D, K370T, | S364K | FIG. 30D of US |
| | | E357Q | | 2016/0355600 |
| Fc 110 | Heterodimerization | L368D, K370T, | S364K | FIG. 30D of US |
| | | Q347E, E357L | | 2016/0355600 |
| Fc 111 | Heterodimerization | L368D, K370T, | S364K | FIG. 30D of US |
| | | Q347E, E357Q | | 2016/0355600 |
| Fc 112 | Heterodimerization | L368E, K370T, | S364K | FIG. 30D of US |
| | | Q347E | | 2016/0355600 |
| Fc 113 | Heterodimerization | L368E, K370T, | S364K | FIG. 30D of US |
| | | E357L | | 2016/0355600 |
| Fc 114 | Heterodimerization | L368E, K370T, | S364K | FIG. 30D of US |
| | | E357Q | | 2016/0355600 |
| Fc 115 | Heterodimerization | L368E, K370T, | S364K | FIG. 30D of US |
| | | Q347E, E357L | | 2016/0355600 |
| Fc 116 | Heterodimerization | L368E, K370T, | S364K | FIG. 30D of US |
| | | Q347E, E357Q | | 2016/0355600 |
| Fc 117 | Heterodimerization | T411E, Q362E | D401K, T411K | FIG. 30D of US |
| | | | | 2016/0355600 |
| Fc 118 | Heterodimerization | T411E, N390D | D401K, T411K | FIG. 30D of US |
| | | | | 2016/0355600 |
| Fc 119 | Heterodimerization | T411E, Q362E | D401R, T411R | FIG. 30D of US |
| | | | | 2016/0355600 |
| Fc 120 | Heterodimerization | T411E, N390D | D401R, T411R | FIG. 30D of US |
| | | | | 2016/0355600 |
| Fc 121 | Heterodimerization | Y407T | T366Y | FIG. 30D of US |
| | | | | 2016/0355600 |
| Fc 122 | Heterodimerization | F405A | T394W | FIG. 30D of US |
| | | | | 2016/0355600 |
| Fc 123 | Heterodimerization | T366Y, F405A | T394W, Y407T | FIG. 30D of US |
| | | | | 2016/0355600 |
| Fc 124 | Heterodimerization | T3665, L368A, | T366W | FIG. 30D of US |
| | | Y407V | | 2016/0355600 |
| Fc 125 | Heterodimerization | T366S, L368A, | T366W, S354C | FIG. 30D of US |
| | | Y407V, Y349C | | 2016/0355600 |
| Fc 126 | Heterodimerization | K392D, K409D | E356K.D399K | FIG. 30E of US |
| | | | | 2016/0355600 |
| Fc 127 | Heterodimerization | K370D, K392D, | E356K, E357K, | FIG. 30E of US |
| | | K409D | D399K | 2016/0355600 |
| Fc 128 | Heterodimerization | I199T, N203D, | Q196K, L99T, | FIG. 30E of US |
| | | K247Q, R355Q, | P217R, P228R, | 2016/0355600 |
| | | N384S, K392N, | N276K | |
| | | V397M, Q419E, | | |
| | | K447 | | |
| Fc 129 | Heterodimerization | I199T, N203D, | Q196K, L99T, | FIG. 30E of US |
| | | K247Q, R355Q, | N276K | 2016/0355600 |
| | | N384S, K392N, | | |
| | | V397M, Q419E, | | |
| | | K447 | | |
| Fc 130 | Heterodimerization | N384S, K392N, | N276K | FIG. 30E of US |
| | | V397M, Q419E | | 2016/0355600 |
| Fc 131 | Heterodimerization | D221E, P228E, | D221R, P228R, | FIG. 30E of US |
| | | L368E | K409R | 2016/0355600 |
| Fc 132 | Heterodimerization | C220E, P228E, | C220R, E224R, | FIG. 30E of US |
| | | L368E | P228R, K409R | 2016/0355600 |
| Fc 133 | Heterodimerization | F405L | K409R | FIG. 30E of US |
| | | | | 2016/0355600 |
| Fc 134 | Heterodimerization | T366I, K392M, | F405A, Y407V | FIG. 30E of US |
| | | T394W | | 2016/0355600 |
| Fc 135 | Heterodimerization | T366V, K409F | L351Y, Y407A | FIG. 30E of US |
| | | | | 2016/0355600 |
| Fc 136 | Heterodimerization | T366A, K392E, | D399R, S400R, | FIG. 30E of US |
| | | K409F, T411E | Y407A | 2016/0355600 |
| Fc 137 | Heterodimerization | L351K | L351E | FIG. 30E of US |
| | | | | 2016/0355600 |
| Fc 138 | Heterodimerization | I199T, N203D, | Q196K, L199T, | FIG. 30E of US |
| | | K247Q, R355Q, | P217R, P228R, | 2016/0355600 |
| | | Q419E, K447 | N276K | |
| Fc 139 | Heterodimerization | I199T, N203D, | Q196K, I199T, | FIG. 30E of US |
| | | K247Q, R355Q, | N276K | 2016/0355600 |
| | | Q419E, K447 | | |
| Fc 140 | Heterodimerization | I199T, N203D, | | FIG. 30E of US |
| | | K274Q, R355Q, | | 2016/0355600 |
| | | N384S, K392N, | | |
| | | V397M, Q419E | | |
| | | DEL447 | | |
| Fc 141 | Heterodimerization | N208D, Q295E | | FIG. 30E of US |
| | | N384D, Q418E | | 2016/0355600 |
| | | N421D | | |
| Fc 142 | Heterodimerization | N208D, Q295E | | FIG. 30E of US |
| | | Q418E, N421D | | 2016/0355600 |
| Fc 143 | Heterodimerization | Q196K, I199T | | FIG. 30E of US |
| | | P217R, P228R | | 2016/0355600 |
| | | N276K | | |
| Fc 144 | Heterodimerization | Q196K, I199T | | FIG. 30E of US |
| | | N276K | | 2016/0355600 |
| Fc 145 | Heterodimerization | E269Q, E272Q | | FIG. 30E of US |
| | | E283Q, E357Q | | 2016/0355600 |
| Fc 146 | Heterodimerization | E269Q, E272Q | | FIG. 30E of US |
| | | E283Q, | | 2016/0355600 |
| Fc 147 | Heterodimerization | E269Q, E272Q | | FIG. 30E of US |
| | | | | 2016/0355600 |
| Fc 148 | Heterodimerization | E269Q, E283Q | | FIG. 30E of US |
| | | | | 2016/0355600 |
| Fc 149 | Heterodimerization | E272Q, E283Q | | FIG. 30E of US |
| | | | | 2016/0355600 |
| Fc 150 | Heterodimerization | E269Q | | FIG. 30E of US |
| | | | | 2016/0355600 |

7.4.1.5.1. Steric Variants

CD3 binding molecules (e.g., MBMs) can comprise one or more, e.g., a plurality, of modifications to one or more of the constant domains of an Fc domain, e.g., to the CH3 domains. In one example, a CD3 binding molecule (e.g., MBM) of the present disclosure comprises two polypeptides that each comprise a heavy chain constant domain of an antibody, e.g., a CH2 or CH3 domain. In an example, the two heavy chain constant domains, e.g., the CH2 or CH3 domains of the CD3 binding molecule (e.g., MBM) comprise one or more modifications that allow for a heterodimeric association between the two chains. In one aspect, the one or more modifications are disposed on CH2 domains of the two heavy chains. In one aspect, the one or more modifications are disposed on CH3 domains of at least two polypeptides of the CD3 binding molecule (e.g., MBM).

One mechanism for Fc heterodimerization is generally referred to as “knobs and holes” or “knobs-into-holes”. These terms refer to amino acid mutations that create steric influences to favor formation of Fc heterodimers over Fc homodimers, as described in, e.g., Ridgway et al., 1996, Protein Engineering 9(7):617; Atwell et al., 1997, J. Mol. Biol. 270:26; U.S. Pat. No. 8,216,805. Knob-in-hole mutations can be combined with other strategies to improve heterodimerization.

In one aspect, the one or more modifications to a first polypeptide of the CD3 binding molecule (e.g., MBM) comprising a heavy chain constant domain can create a “knob” and the one or more modifications to a second polypeptide of the CD3 binding molecule (e.g., MBM) creates a “hole,” such that heterodimerization of the polypeptide of the CD3 binding molecule (e.g., MBM) comprising a heavy chain constant domain causes the “knob” to interface (e.g., interact, e.g., a CH2 domain of a first polypeptide interacting with a CH2 domain of a second polypeptide, or a CH3 domain of a first polypeptide interacting with a CH3 domain of a second polypeptide) with the “hole.” As the term is used herein, a “knob” refers to at least one amino acid side chain which projects from the interface of a first polypeptide of the CD3 binding molecule (e.g., MBM) comprising a heavy chain constant domain and is therefore positionable in a compensatory “hole” in the interface with a second polypeptide of the CD3 binding molecule (e.g., MBM) comprising a heavy chain constant domain so as to stabilize the heteromultimer, and thereby favor heteromultimer formation over homomultimer formation, for example. The knob can exist in the original interface or can be introduced synthetically (e.g. by altering nucleic acid encoding the interface). The preferred import residues for the formation of a knob are generally naturally occurring amino acid residues and are preferably selected from arginine (R), phenylalanine (F), tyrosine (Y) and tryptophan O). Most preferred are tryptophan and tyrosine. In the preferred embodiment, the original residue for the formation of the protuberance has a small side chain volume, such as alanine, asparagine, aspartic acid, glycine, serine, threonine or valine.

A “hole” refers to at least one amino acid side chain that is recessed from the interface of a second polypeptide of the CD3 binding molecule (e.g., MBM) comprising a heavy chain constant domain and therefore accommodates a corresponding knob on the adjacent interfacing surface of a first polypeptide of the CD3 binding molecule (e.g., MBM) comprising a heavy chain constant domain. The hole can exist in the original interface or can be introduced synthetically (e.g. by altering nucleic acid encoding the interface). The preferred import residues for the formation of a hole are usually naturally occurring amino acid residues and are preferably selected from alanine (A), serine (S), threonine (T) and valine (V). Most preferred are serine, alanine or threonine. In the preferred embodiment, the original residue for the formation of the hole has a large side chain volume, such as tyrosine, arginine, phenylalanine or tryptophan.

In a preferred embodiment, a first CH3 domain is modified at residue 366, 405 or 407 to create either a “knob” or a hole” (as described above), and the second CH3 domain that heterodimerizes with the first CH3 domain is modified at: residue 407 if residue 366 is modified in the first CH3 domain, residue 394 if residue 405 is modified in the first CH3 domain, or residue 366 if residue 407 is modified in the first CH3 domain to create a “hole” or “knob” complementary to the “knob” or “hole” of the first CH3 domain.

In another preferred embodiment, a first CH3 domain is modified at residue 366, and the second CH3 domain that heterodimerizes with the first CH3 domain is modified at residues 366, 368 and/or 407, to create a “hole” or “knob” complementary to the “knob” or “hole” of the first CH3 domain. In one embodiment, the modification to the first CH3 domain introduces a tyrosine (Y) residue at position 366. In an embodiment, the modification to the first CH3 is T366Y. In one embodiment, the modification to the first CH3 domain introduces a tryptophan (W) residue at position 366. In an embodiment, the modification to the first CH3 is T366W. In some embodiments, the modification to the second CH3 domain that heterodimerizes with the first CH3 domain modified at position 366 (e.g., has a tyrosine (Y) or tryptophan (N) introduced at position 366, e.g., comprises the modification T366Y or T366W), comprises a modification at position 366, a modification at position 368 and a modification at position 407. In some embodiments, the modification at position 366 introduces a serine (S) residue, the modification at position 368 introduces an alanine (A), and the modification at position 407 introduces a valine (V). In some embodiments, the modifications comprise T366S, L368A and Y407V. In one embodiment the first CH3 domain of the multispecific molecule comprises the modification T366Y, and the second CH3 domain that heterodimerizes with the first CH3 domain comprises the modifications T366S, L368A and Y407V, or vice versa. In one embodiment the first CH3 domain of the multispecific molecule comprises the modification T366W, and the second CH3 domain that heterodimerizes with the first CH3 domain comprises the modifications T366S, L368A and Y407V, or vice versa.

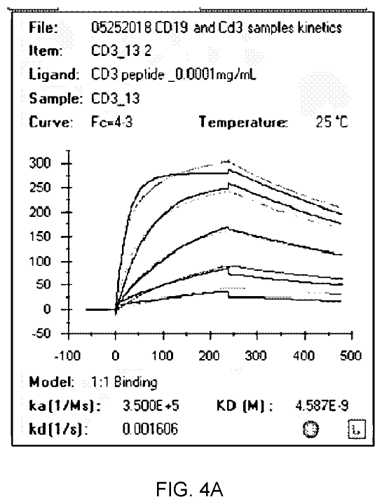

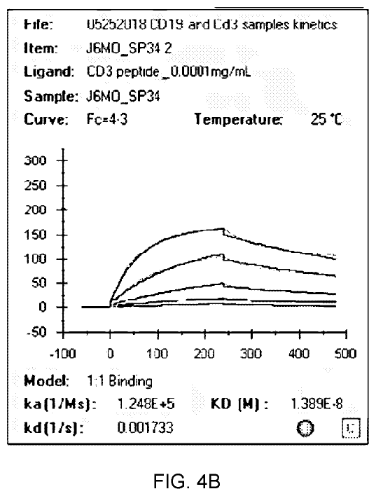

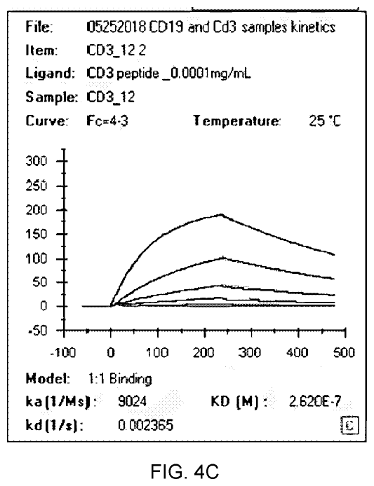

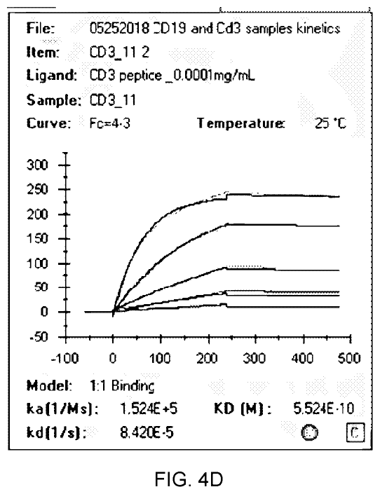

Additional steric or “skew” (e.g., knob in hole) modifications are described in PCT publication no. WO2014/145806 (for example, FIG. 3, FIG. 4 and FIG. 12 of WO2014/145806), PCT publication no. WO2014/110601, and PCT publication no. WO 2016/086186, WO 2016/086189, WO 2016/086196 and WO 2016/182751 the contents of which are incorporated herein in their entireties. An example of a KIH variant comprises a first constant chain comprising a L368D and a K370S modification, paired with a second constant chain comprising a S364K and E357Q modification.

Additional knob in hole modification pairs suitable for use in any of the CD3 binding molecules (e.g., MBMs) of the present disclosure are further described in, for example, WO1996/027011, and Merchant et al., 1998, Nat. Biotechnol., 16:677-681.

In further embodiments, the CH3 domains can be additionally modified to introduce a pair of cysteine residues. Without being bound by theory, it is believed that the introduction of a pair of cysteine residues capable of forming a disulfide bond provide stability to heterodimerized CD3 binding molecules (e.g., MBMs) comprising paired CH3 domains. In some embodiments, the first CH3 domain comprises a cysteine at position 354, and the second CH3 domain that heterodimerizes with the first CH3 domain comprises a cysteine at position 349. In some embodiments, the first CH3 domain comprises a cysteine at position 354 (e.g., comprises the modification S354C) and a tyrosine (Y) at position 366 (e.g., comprises the modification T366Y), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V). In some embodiments, the first CH3 domain comprises a cysteine at position 354 (e.g., comprises the modification S354C) and a tryptophan (W) at position 366 (e.g., comprises the modification T366W), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V).

An additional mechanism that finds use in the generation of heterodimers is sometimes referred to as “electrostatic steering” as described in Gunasekaran et al., 2010, J. Biol. Chem. 285(25):19637. This is sometimes referred to herein as “charge pairs”. In this embodiment, electrostatics are used to skew the formation towards heterodimerization. As a skilled artisan will appreciate, these can also have an effect on pl, and thus on purification, and thus could in some cases also be considered pl variants. However, as these were generated to force heterodimerization and were not used as purification tools, they are classified as “steric variants”. These include, but are not limited to, D221E/P228E/L368E paired with D221R/P228R/K409R and C220E/P228E/368E paired with C220R/E224R/P228R/K409R.

Additional variants that can be combined with other variants, optionally and independently in any amount, such as pl variants outlined herein or other steric variants that are shown in FIG. 37 of US 2012/0149876.

In some embodiments, the steric variants outlined herein can be optionally and independently incorporated with any pl variant (or other variants such as Fc variants, FcRn variants) into one or both Fc regions, and can be independently and optionally included or excluded from the CD3 binding molecules.

A list of suitable skew variants is found in Table 5 showing some pairs of particular utility in many embodiments. Of particular use in many embodiments are the pairs of sets including, but not limited to, S364K/E357Q:L368D/K370S; L368D/K370S:S364K; L368E/K370S:S364K; T411T/E360E/Q362E:D401K; L368D/K370S:S364K/E357L; and K370S:S364K/E357Q. In terms of nomenclature, the pair “S364K/E357Q:L368D/K370S” means that one of the Fc regions has the double variant set S364K/E357Q and the other has the double variant set L368D/K370S.

TABLE 5
Exemplary skew variants

| Fc region 1 | Fc region 2 |
|---|---|
| F405A | T394F |
| S364D | Y349K |
| S364E | L368K |
| S364E | Y349K |
| S364F | K370G |
| S364H | Y349K |
| S364H | Y349T |
| S364Y | K370G |
| T411K | K370E |
| V397S/F405A | T394F |
| K370R/T411K | K370E/T411E |
| L351E/S364D | Y349K/L351K |
| L351E/S364E | Y349K/L351K |
| L351E/T366D | L351K/T366K |
| P395T/V397S/F405A | T394F |
| S364D/K370G | S364Y/K370R |
| S364D/T394F | Y349K/F405A |
| S364E/F405A | Y349K/T394F |
| S364E/F405S | Y349K/T394Y |
| S364E/T411E | Y349K/D401K |
| S364H/D401K | Y349T/T411E |
| S364H/F405A | Y349T/T394F |
| S364H/T394F | Y349T/F405A |
| Y349C/S364E | Y349K/S354C |
| L351E/S364D/F405A | Y349K/L351K/T394F |
| L351K/S364H/D401K | Y349T/L351E/T411E |
| S364E/T411E/F405A | Y349K/T394F/D401K |
| S364H/D401K/F405A | Y349T/T394F/T411E |
| S364H/F405A/T411E | Y349T/T394F/D401K |
| K370E/T411D | T411K |
| L368E/K409E | L368K |
| Y349T/T394F/S354C | S364H/F405A/Y349C |
| T411E | D401K |
| T411E | D401R/T411R |
| Q347E/K360E | Q347R |
| L368E | S364K |
| L368E/K370S | S364K |
| L368E/K370T | S364K |
| L368E/D401R | S364K |
| L368E/D401N | S364K |
| L368E | E357S/S364K |
| L368E | S364K/K409E |
| L368E | S364K/K409V |
| L368D | S364K |
| L368D/K370S | S364K |
| L368D/K370S | S364K/E357L |
| L368D/K370S | S364K/E357Q |
| T411E/K360E/Q362E | D401K |
| K370S | S364K |
| L368E/K370S | S364K/E357Q |
| K370S | S364K/E357Q |
| T411E/K360D | D401K |
| T411E/K360E | D401K |
| T411E/Q362E | D401K |
| T411E/N390D | D401K |
| T411E | D401K/Q347K |
| T411E | D401K/Q347R |
| T411E/K360D/Q362E | D401K |
| K392D/K409D | E356K/D399K |
| K370D/K392D/K409D | E356K/E357K/D399K |
| I199T/N203D/K247Q/R355Q/N384S/K392N/ | Q196K/1199T/P217R/ |
| V397M/Q419E/K447_{—} | P228R/N276K |
| I199T/N203D/K247Q/R355Q/N384S/K392N/ | Q196K/I199T/N276K |
| V397M/Q419E/K447_{—} | |
| N384S/K392N/V397M/Q419E | N276K |
| D221E/P228E/L368E | D221R/P228R/K409R |
| C220E/P228E/L368E | C220R/E224R/P228R/ |
| | K409R |
| F405L | K409R |
| T366I/K392M/T394W | F405A/Y407V |
| T366V/K409F | L351Y/Y407A |
| T366A/K392E/K409F/T411E | D399R/S400R/Y407A |
| L351K | L351E |
| I199T/N203D/K247Q/R355Q/Q419E/K447_{—} | Q196K/1199T/P217R/ |
| | P228R/N276K |
| I199T/N203D/K247Q/R355Q/Q419E/K447_{—} | Q196K/I199T/N276K |
| I199T N203D K274Q R355Q N384S K392N | |
| V397M Q419E DEL447 | |
| N208D Q295E N384D Q418E N421D | |
| N208D Q295E Q418E N421D | |
| Q196K I199T P217R P228R N276K | |
| Q196K I199T N276K | |
| E269Q E272Q E283Q E357Q | |
| E269Q E272Q E283Q | |
| E269Q E272Q | |
| E269Q E283Q | |
| E272Q E283Q | |
| E269Q | |
| T411E/K360E/N390D | D401K |
| T411E/Q362E/N390D | D401K |
| T411E/Q347R | D401K/K360D |
| T411E/Q347R | D401K/K360E |
| T411E/K360 | D401K/Q347K |
| T411E/K360D | D401K/Q347R |
| T411E/K360E | D401K/Q347K |
| T411E/K360E | D401K/Q347R |
| T411E/S364K | D401K/K370S |
| T411E/K370S | D401K/S364K |
| Q347E | E357Q |
| Q347E | E357Q/Q362K |
| K360D/Q362E | Q347R |
| K360D/Q362E | D401K |
| K360D/Q362E | Q347R/D401K |
| K360E/Q362E | Q347R |
| K360E/Q362E | D401K |
| K360E/Q362E | Q347R/D401K |
| Q362E/N390D | D401K |
| Q347E/K360D | D401N |
| K360D | Q347R/N390K |
| K360D | N390K/D401N |
| K360E | Y349H |
| K370S/Q347E | S364K |
| K370S/E357L | S364K |
| K370S/E357Q | S364K |
| K370S/Q347E/E357L | S364K |
| K370S/Q347E/E357Q | S364K |
| L368D/K370S/Q347E | S364K |
| L368D/K370S/E357L | S364K |
| L368D/K370S/E357Q | S364K |
| L368D/K370S/Q347E/E357L | S364K |
| L368D/K370S/Q347E/E357Q | S364K |
| L368E/K370S/Q347E | S364K |
| L368E/K370S/E357L | S364K |
| L368E/K370S/E357Q | S364K |
| L368E/K370S/Q347E/E357L | S364K |
| L368E/K370S/Q347E/E357Q | S364K |
| L368D/K370T/Q347E | S364K |
| L368D/K370T/E357L | S364K |
| L368D/K370T/E357Q | S364K |
| L368D/K370T/Q347E/E357L | S364K |
| L368D/K370T/Q347E/E357Q | S364K |
| L368E/K370T/Q347E | S364K |
| L368E/K370T/E357L | S364K |
| L368E/K370T/E357Q | S364K |
| L368E/K370T/Q347E/E357L | S364K |
| L368E/K370T/Q347E/E357Q | S364K |
| T411E/Q362E | D401K/T411K |
| T411E/N390D | D401K/T411K |
| T411E/Q362E | D401R/T411R |
| T411E/N390D | D401R/T411R |
| Y407T | T366Y |
| F405A | T394W |
| T366Y/F405A | T394W/Y407T |
| Y407A | T366W |
| T366S/L368A/Y407V | T366W |
| T366S/L368A/Y407V/Y349C | T366W/S354C |
| K392D/K409D | E356K/D399K |
| K370D/K392D/K409D | E356K/E357K/D399K |
| I199T/N203D/K247Q/R355Q/N384S/K392N/ | Q196K/I199T/P217R/ |
| V397M/Q419E/K447 | P228R/N276K |
| I199T/N203D/K247Q/R355Q/N384S/K392N/ | Q196K/I199T/N276K |
| V397M/Q419E/K447 | |
| N384S/K392N/V397M/Q419E | N276K |
| D221E/P228E/L368E | D221R/P228R/K409R |
| C220E/P228E/L368E | C220R/E224R/P228R/ |
| | K409R |
| F405L | K409R |
| T366I/K392M/T394W | F405A/Y407V |
| T366V/K409F | L351Y/Y407A |
| T366A/K392E/K409F/T411E | D399R/S400R/Y407A |
| L351K | L351E |
| I199T/N203D/K247Q/R355Q/Q419E/K447_{—} | Q196K/I199T/P217R/ |
| | P228R/N276K |
| I199T/N203D/K247Q/R355Q/Q419E/K447_{—} | Q196K/I199T/N276K |
| I199T N203D K274Q R355Q N384S K392N | |
| V397M Q419E DEL447 | |
| N208D Q295E N384D Q418E N421D | |
| Q295E N384D Q418E N421D | |
| N208D Q295E Q418E N421D | |
| Q295EQ418E N421D | |
| Q196K I199T P217R P228R N276K | |
| Q196K I199T N276K | |
| E269Q E272Q E283Q E357Q | |
| E269Q E272Q E283Q | |
| E269Q E272Q | |
| E269Q E283Q | |
| E272Q E283Q | |
| E269Q | |

In some embodiments, a CD3 binding molecule comprises a first Fc region and a second Fc region. In some embodiments, the first Fc region comprises the following mutations: L368D and K370S, and the second Fc region comprises the following mutations: S364K and E357Q. In some embodiments, the first Fc region comprises the following mutations: S364K and E357Q, and the second Fc region comprises the following mutations: L368D and K370S.

7.4.1.5.2. Alternative Knob and Hole: IgG Heterodimerization

Heterodimerization of polypeptide chains of a CD3 binding molecule (e.g., MBM) comprising paired CH3 domains can be increased by introducing one or more modifications in a CH3 domain which is derived from the IgG1 antibody class. In an embodiment, the modifications comprise a K409R modification to one CH3 domain paired with F405L modification in the second CH3 domain. Additional modifications can also, or alternatively, be at positions 366, 368, 370, 399, 405, 407, and 409. Preferably, heterodimerization of polypeptides comprising such modifications is achieved under reducing conditions, e.g., 10-100 mM 2-MEA (e.g., 25, 50, or 100 mM 2-MEA) for 1-10, e.g., 1.5-5, e.g., 5, hours at 25-37 C, e.g., 25 C or 37 C.

The amino acid replacements described herein can be introduced into the CH3 domains using techniques which are well known in the art (see, e.g., McPherson, ed., 1991, Directed Mutagenesis: a Practical Approach; Adelman et al., 1983, DNA, 2:183).

The IgG heterodimerization strategy is further described in, for example, WO2008/119353, WO2011/131746, and WO2013/060867.

In any of the embodiments described in this Section, the CH3 domains can be additionally modified to introduce a pair of cysteine residues as described in Section 7.4.1.5.1.

7.4.1.5.3. Pl (Isoelectric Point) Variants

In general, as will be appreciated by a skilled artisan, there are two general categories of pl variants: those that increase the pl of the protein (basic changes) and those that decrease the pl of the protein (acidic changes). As described herein, all combinations of these variants can be done: one Fc region can be wild type, or a variant that does not display a significantly different pl from wild-type, and the other can be either more basic or more acidic. Alternatively, each Fc region is changed, one to more basic and one to more acidic.

Exemplary combinations of pl variants are shown in Table 6. As outlined herein and shown in Table 6, these changes are shown relative to IgG1, but all isotypes can be altered this way, as well as isotype hybrids. In the case where the heavy chain constant domain is from IgG2-4, R133E and R133Q can also be used.

TABLE 6
Exemplary pI Variant Combinations

| Variant constant reqion | Substitutions |
|---|---|
| pI_ISO(−) | I199T N203D K274Q R355Q N384S |
| | K392N V397M Q419E DEL447 |
| pI_(−)_isosteric_A | N208D Q295E N384D Q418E |
| | N421D |
| pI_(−)_isosteric A-Fc only | Q295E N384D Q418E N421D |
| pI_(−)_isosteric_B | N208D Q295E Q418E N421D |
| pI_(−)_isosteric_B-Fc only | Q295E Q418E N421D |
| pI_ISO(+RR) | Q196K I199T P217R P228R N276K |
| pI_ISO(+) | Q196K I199T N276K |
| pI_(+)_isosteric_A | E269Q E272Q E283Q E357Q |
| pI_(+)_isosteric_B | E269Q E272Q E283Q |
| pI_(+)_isosteric_E269Q/E272Q | E269Q E272Q |
| pI_(+)_isosteric_E269Q/E283Q | E269Q E283Q |
| pI_(+)_isosteric_E272Q/E283Q | E272Q E283Q |
| pI_(+)_isosteric_E269Q | E269Q |

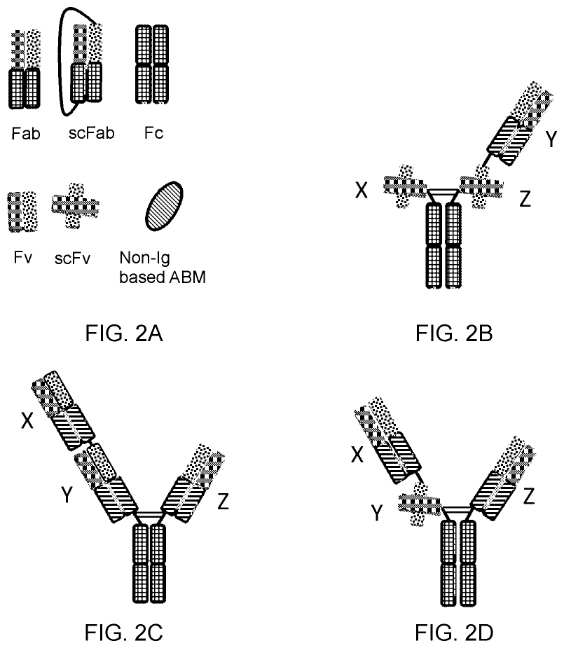

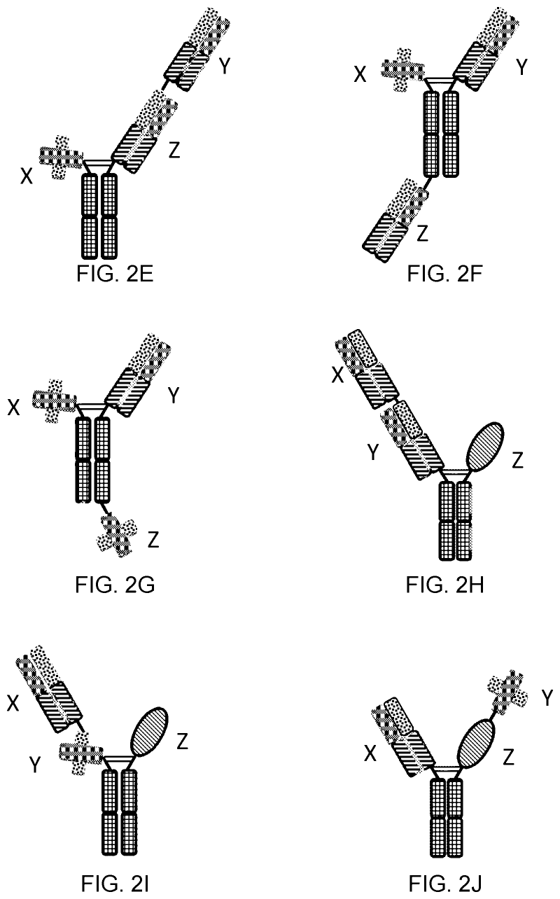

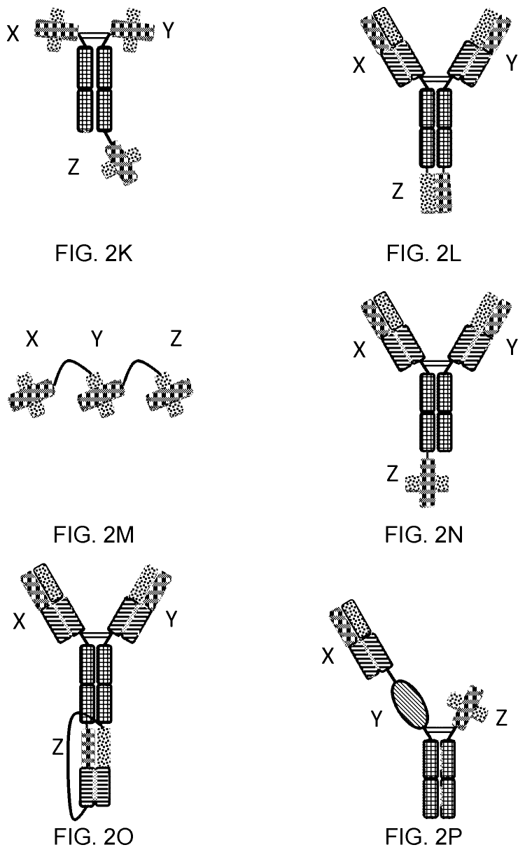

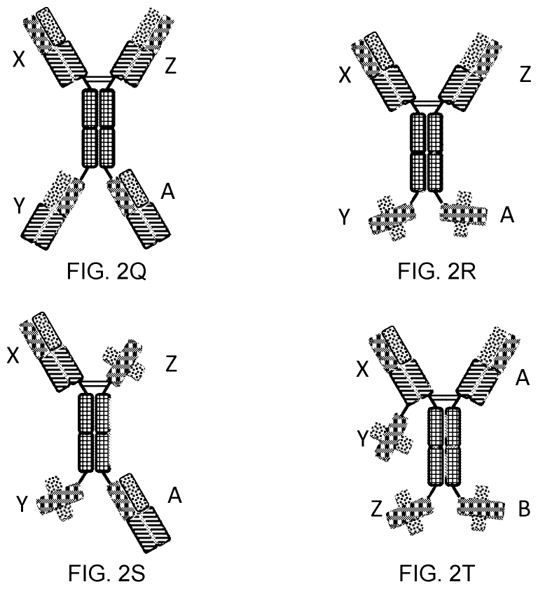

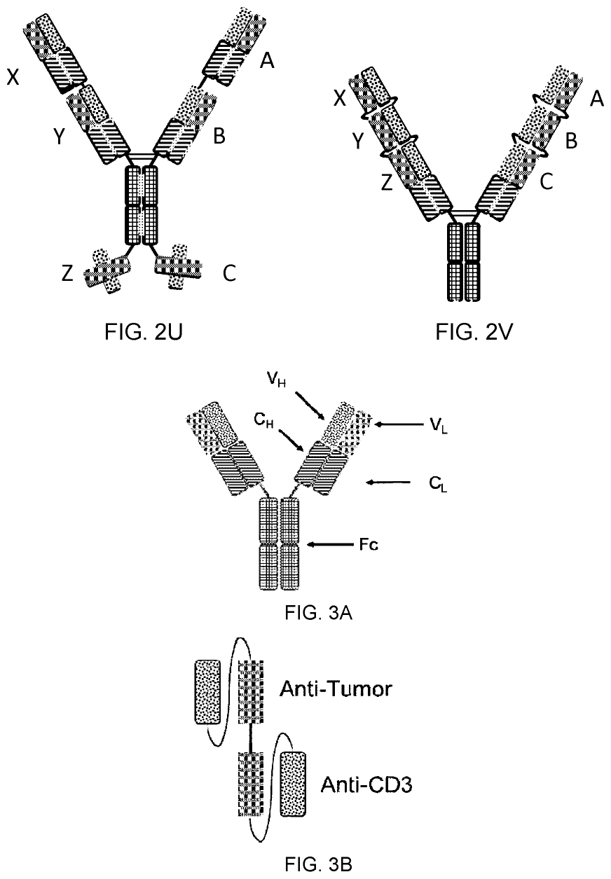

In one embodiment, for example in the FIG. 1B-1W, FIG. 1Y-1AH, FIG. 2B-2L, and FIG. 2N-2V formats, a combination of pl variants has one Fc region (the negative Fab side) comprising 208D/295E/384D/418E/421D variants (N208D/Q295E/N384D/Q418E/N421D when relative to human IgG1) and a second Fc region (the positive scFv side) comprising a positively charged scFv linker, e.g., L36 (described in Section 7.4.3). However, as will be appreciated by a skilled artisan, the first Fc region includes a CH1 domain, including position 208. Accordingly, in constructs that do not include a CH1 domain (for example for MBMs that do not utilize a CH1 domain as one of the domains, for example in a format depicted in FIG. 2K), a negative pl variant Fc set can include 295E/384D/418E/421D variants (Q295E/N384D/Q418E/N421D when relative to human IgG1).

In some embodiments, a first Fc region has a set of substitutions from Table 6 and a second Fc region is connected to a charged linker (e.g., selected from those described in Section 7.4.3).

In some embodiments, the CD3 binding molecule of the present disclosure comprises a first Fc region and a second Fc region. In some embodiments, the first Fc region comprises the following mutations: N208D, Q295E, N384D, Q418E, and N421D. In some embodiments, the second Fc region comprises the following mutations: N208D, Q295E, N384D, Q418E, and N421D.

7.4.1.5.4. Isotopic Variants

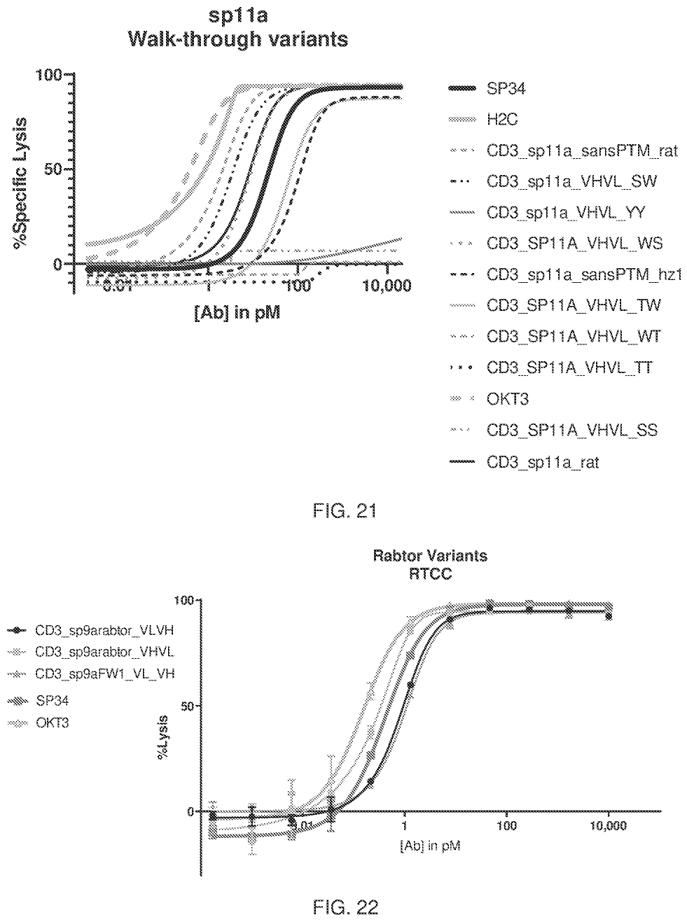

In addition, many embodiments of the disclosure rely on the “importation” of pl amino acids at particular positions from one IgG isotype into another, thus reducing or eliminating the possibility of unwanted immunogenicity being introduced into the variants. A number of these are shown in FIG. 21 of US Publ. 2014/0370013. That is, IgG1 is a common isotype for therapeutic antibodies for a variety of reasons, including high effector function. However, the heavy constant region of IgG1 has a higher pl than that of IgG2 (8.10 versus 7.31). By introducing IgG2 residues at particular positions into the IgG1 backbone, the pl of the resulting Fc region is lowered (or increased) and additionally exhibits longer serum half-life. For example, IgG1 has a glycine (pl 5.97) at position 137, and IgG2 has a glutamic acid (pl 3.22); importing the glutamic acid will affect the pl of the resulting protein. As is described below, a number of amino acid substitutions are generally required to significantly affect the pl of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pl amino acid to a lower pl amino acid), or to allow accommodations in structure for stability, as is further described below.

In addition, by pl engineering both the heavy and light constant domains of a CD3 binding molecule comprising two half antibodies, significant changes in each half antibody can be seen. Having the pls of the two half antibodies differ by at least 0.5 can allow separation by ion exchange chromatography or isoelectric focusing, or other methods sensitive to isoelectric point.

7.4.1.5.5. Calculating pl

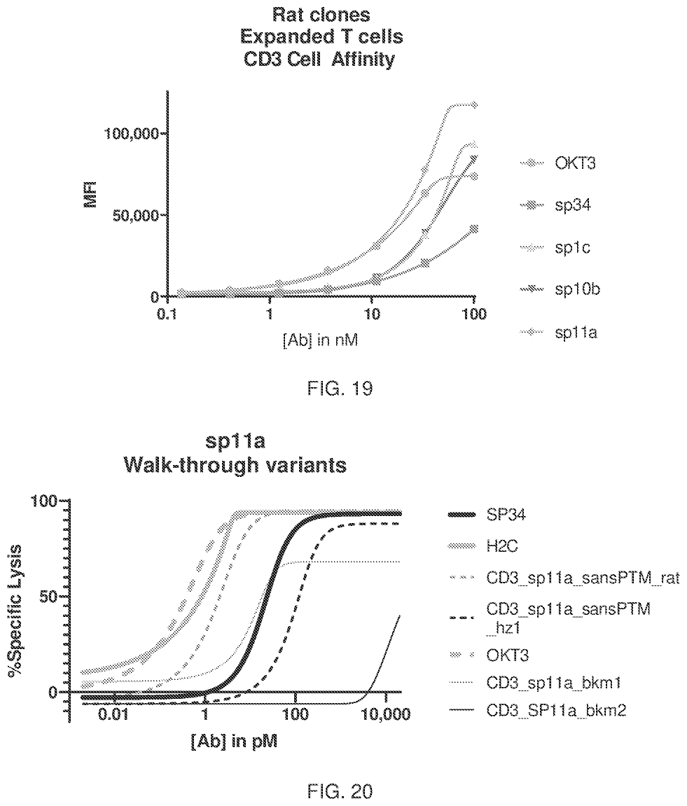

The pl of a half antibody comprising an Fc region and an ABM or ABM chain can depend on the pl of the variant heavy chain constant domain and the pl of the total half antibody, including the variant heavy chain constant domain and ABM or ABM chain. Thus, in some embodiments, the change in pl is calculated on the basis of the variant heavy chain constant domain, using the chart in the FIG. 19 of US Pub. 2014/0370013. As discussed herein, which half antibody to engineer is generally decided by the inherent pl of the half antibodies. Alternatively, the pl of each half antibody can be compared.

7.4.1.5.6. Pl Variants that Also Confer Better FcRn In Vivo Binding

In the case where a pl variant decreases the pl of an Fc region, it can have the added benefit of improving serum retention in vivo.

pl variant Fc regions are believed to provide longer half-lives to antigen binding molecules in vivo, because binding to FcRn at pH 6 in an endosome sequesters the Fc (Ghetie and Ward, 1997, Immunol Today. 18(12): 592-598). The endosomal compartment then recycles the Fc to the cell surface. Once the compartment opens to the extracellular space, the higher pH ˜7.4, induces the release of Fc back into the blood. In mice, DaII' Acqua et al. showed that Fc mutants with increased FcRn binding at pH 6 and pH 7.4 actually had reduced serum concentrations and the same half life as wild-type Fc (Dall'Acqua et al., 2002, J. Immunol. 169:5171-5180). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

It has been suggested that antibodies with variable regions that have lower isoelectric points can also have longer serum half-lives (Igawa et al., 2010, PEDS. 23(5): 385-392). However, the mechanism of this is still poorly understood. Moreover, variable regions differ from antibody to antibody. Constant region variants with reduced pl and extended half-life would provide a more modular approach to improving the pharmacokinetic properties of CD3 binding molecules, as described herein.

7.4.1.5.7. Polar Bridge

Heterodimerization of polypeptide chains of CD3 binding molecules (e.g., MBMs) comprising an Fc domain can be increased by introducing modifications based on the “polar-bridging” rationale, which is to make residues at the binding interface of the two polypeptide chains to interact with residues of similar (or complimentary) physical property in the heterodimer configuration, while with residues of different physical property in the homodimer configuration. In particular, these modifications are designed so that, in the heterodimer formation, polar residues interact with polar residues, while hydrophobic residues interact with hydrophobic residues. In contrast, in the homodimer formation, residues are modified so that polar residues interact with hydrophobic residues. The favorable interactions in the heterodimer configuration and the unfavorable interactions in the homodimer configuration work together to make it more likely for Fc regions to form heterodimers than to form homodimers.

In an exemplary embodiment, the above modifications are generated at one or more positions of residues 364, 368, 399, 405, 409, and 411 of a CH3 domain.

In some embodiments, one or more modifications selected from S364L, T366V, L368Q, N399K, F405S, K409F and R411K are introduced into one of the two CH3 domains. One or more modifications selected from Y407F, K409Q and T411N can be introduced into the second CH3 domain.

In another embodiment, one or more modifications selected from a group consisting of S364L, T366V, L368Q, D399K, F405S, K409F and T411K are introduced into one CH3 domain, while one or more modifications selected from Y407F, K409Q and T411D are introduced into the second CH3 domain.

In one exemplary embodiment, the original residue of threonine at position 366 of one CH3 domain is replaced by valine, while the original residue of tyrosine at position 407 of the other CH3 domain is replaced by phenylalanine.

In another exemplary embodiment, the original residue of serine at position 364 of one CH3 domain is replaced by leucine, while the original residue of leucine at position 368 of the same CH3 domain is replaced by glutamine.

In yet another exemplary embodiment, the original residue of phenylalanine at position 405 of one CH3 domain is replaced by serine and the original residue of lysine at position 409 of this CH3 domain is replaced by phenylalanine, while the original residue of lysine at position 409 of the other CH3 domain is replaced by glutamine.

In yet another exemplary embodiment, the original residue of aspartic acid at position 399 of one CH3 domain is replaced by lysine, and the original residue of threonine at position 411 of the same CH3 domain is replaced by lysine, while the original residue of threonine at position 411 of the other CH3 domain is replaced by aspartic acid.

The amino acid replacements described herein can be introduced into the CH3 domains using techniques which are well known in the art (see, e.g., McPherson, ed., 1991, Directed Mutagenesis: a Practical Approach; Adelman et al., 1983, DNA, 2:183). The polar bridge strategy is described in, for example, WO2006/106905, WO2009/089004 and K. Gunasekaran, et al. (2010) The Journal of Biological Chemistry, 285:19637-19646.

Additional polar bridge modifications are described in, for example, PCT publication no. WO2014/145806 (for example, FIG. 6 of WO2014/145806), PCT publication no. WO2014/110601, and PCT publication no. WO 2016/086186, WO 2016/086189, WO 2016/086196 and WO 2016/182751 the contents of which are incorporated herein in their entireties. An example of a polar bridge variant comprises a constant chain comprising a N208D, Q295E, N384D, Q418E and N421D modification.

In any of the embodiments described herein, the CH3 domains can be additionally modified to introduce a pair of cysteine residues as described in Section 7.4.1.5.1.

Additional strategies for enhancing heterodimerization are described in, for example, WO2016/105450, WO2016/086186, WO2016/086189, WO2016/086196, WO2016/141378, and WO2014/145806, and WO2014/110601. Any of said strategies can be employed in a CD3 binding molecule (e.g., MBM) described herein.

7.4.1.5.8. Combination of Heterodimerization Variants and Other Fc Variants

As will be appreciated by a skilled artisan, all of the recited heterodimerization variants (including skew and/or pl variants) can be optionally and independently combined in any way, as long as the Fc regions of an Fc domain retain their ability to dimerize. In addition, all of these variants can be combined into any of the heterodimerization formats.

In the case of pl variants, while embodiments finding particular use are shown in the Table 6, other combinations can be generated, following the basic rule of altering the pl difference between two Fc regions in an Fc heterodimer to facilitate purification.

In addition, any of the heterodimerization variants, skew and pl, are also independently and optionally combined with Fc ablation variants, Fc variants, FcRn variants, as generally outlined herein.

In some embodiments, a particular combination of skew and pl variants that finds use in the present disclosure is T366S/L368A/Y407V:T366W (optionally including a bridging disulfide, T366S/L368A/Y407V/Y349C:T366W/S354C) with one Fc region comprising Q295E/N384D/Q418E/N481D and the other a positively charged scFv linker (when the format includes an scFv domain). As will be appreciated by a skilled artisan, the “knobs in holes” variants do not change pl, and thus can be used on either one of the Fc regions in an Fc heterodimer.

In some embodiments, first and second Fc regions that find use the present disclosure include the amino acid substitutions S364K/E357Q:L368D/K370S, where the first and/or second Fc region includes the ablation variant substitutions 233P/L234V/L235A/G236del/S267K, and the first and/or second Fc region comprises the pl variant substitutions N208D/Q295E/N384D/Q418E/N421D (pl_(−)-Lisosteric_A).

7.4.2. Hinge Regions

The CD3 binding molecules (e.g., MBMs) can also comprise hinge regions, e.g., connecting an antigen-binding module to an Fc region. The hinge region can be a native or a modified hinge region. Hinge regions are typically found at the N-termini of Fc regions.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions can comprise a complete hinge region derived from an antibody of a different class or subclass from that of the heavy chain Fc region. Alternatively, the modified hinge region can comprise part of a natural hinge or a repeating unit in which each unit in the repeat is derived from a natural hinge region. In a further alternative, the natural hinge region can be altered by converting one or more cysteine or other residues into neutral residues, such as serine or alanine, or by converting suitably placed residues into cysteine residues. By such means, the number of cysteine residues in the hinge region can be increased or decreased. This approach is described further in U.S. Pat. No. 5,677,425 by Bodmer et al. Altering the number of cysteine residues in a hinge region can, for example, facilitate assembly of light and heavy chains, or increase or decrease the stability of a CD3 binding molecule. Other modified hinge regions can be entirely synthetic and can be designed to possess desired properties such as length, cysteine composition and flexibility.

A number of modified hinge regions have already been described for example, in U.S. Pat. No. 5,677,425, WO9915549, WO2005003170, WO2005003169, WO2005003170, WO9825971 and WO2005003171.

Examples of suitable hinge sequences are shown in Table 7.

TABLE 7
Hinge Sequences

| Hinge | Hinge | | SEQ ID |
|---|---|---|---|
| Name | Description | Hinge Sequence | NO: |
| H1 | Human IgA1 | VPSTPPTPSPSTPPTPSPS | SEQ ID |
| | | | NO: 1 |
| H2 | Human IgA2 | VPPPPP | SEQ ID |
| | | | NO: 2 |
| H3 | Human IgD | ESPKAQASSVPTAQPQAEGSLAKATTAPATTRN | SEQ ID |
| | | TGRGGEEKKKEKEKEEQEERETKTP | NO: 3 |
| H4 | Human IgG1 | EPKSCDKTHTCPPCP | SEQ ID |
| | | | NO: 4 |
| H5 | Human IgG2 | ERKCCVECPPCP | SEQ ID |
| | | | NO: 5 |
| H6 | Human IgG3 | ELKTPLGDTTHTCPRCPEPKSCDTPPPCPRCPE | SEQ ID |
| | | PKSCDTPPPCPRCPEPKSCDTPPPCPRCP | NO: 6 |
| H7 | Human IgG4 | ESKYGPPCPSCP | SEQ ID |
| | | | NO: 7 |
| H8 | Human IgG4(P) | ESKYGPPCPPCP | SEQ ID |
| | | | NO: 8 |
| H9 | Engineered v1 | CPPC | SEQ ID |
| | | | NO: 9 |
| H10 | Engineered v2 | CPSC | SEQ ID |
| | | | NO: 10 |
| H11 | Engineered v3 | CPRC | SEQ ID |
| | | | NO: 11 |
| H12 | Engineered v4 | SPPC | SEQ ID |
| | | | NO: 12 |
| H13 | Engineered v5 | CPPS | SEQ ID |
| | | | NO: 13 |
| H14 | Engineered v6 | SPPS | SEQ ID |
| | | | NO: 14 |
| H15 | Engineered v7 | DKTHTCAA | SEQ ID |
| | | | NO: 15 |
| H16 | Engineered v8 | DKTHTCPPCPA | SEQ ID |
| | | | NO: 16 |
| H17 | Engineered v9 | DKTHTCPPCPATCPPCPA | SEQ ID |
| | | | NO: 17 |
| H18 | Engineered v10 | DKTHTCPPCPATCPPCPATCPPCPA | SEQ ID |
| | | | NO: 18 |
| H19 | Engineered v11 | DKTHTCPPCPAGKPTLYNSLVMSDTAGTCY | SEQ ID |
| | | | NO: 19 |
| H20 | Engineered v12 | DKTHTCPPCPAGKPTHVNVSVVMAEVDGTCY | SEQ ID |
| | | | NO: 20 |
| H21 | Engineered v13 | DKTHTCCVECPPCPA | SEQ ID |
| | | | NO: 21 |
| H22 | Engineered v14 | DKTHTCPRCPEPKSCDTPPPCPRCPA | SEQ ID |
| | | | NO: 22 |
| H23 | Engineered v15 | DKTHTCPSCPA | SEQ ID |
| | | | NO: 23 |

In one embodiment, the heavy chain Fc region possesses an intact hinge region at its N-terminus.

In one embodiment the heavy chain Fc region and hinge region are derived from IgG4 and the hinge region comprises the modified sequence CPPC (SEQ ID NO: 9). The core hinge region of human IgG4 contains the sequence CPSC (SEQ ID NO: 10) compared to IgG1 which contains the sequence CPPC (SEQ ID NO: 9). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

7.4.3. ABM Linkers

In certain aspects, the present disclosure provides CD3 binding molecules (e.g., MBMs) comprising at least three ABMs, wherein two or more components of an ABM (e.g., a VH and a VL of a scFv), two or more ABMs, or an ABM and a non-ABM domain (e.g., a dimerization domain such as an Fc region) are connected to one another by a peptide linker. Such linkers are referred to herein an “ABM linkers,” as opposed to the ADC linkers used to attach drugs to CD3 binding molecules (e.g., MBMs) as described, for example, in Section 7.13.2.

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids or from 12 to 20 amino acids. In particular embodiments, a peptide linker is 2 amino acids, 3 amino acids, 4 amino acid, 5 amino acids, 6 amino acids, 7 amino acids, 8 amino acids, 9 amino acids, 10 amino acids, 11 amino acids, 12 amino acids, 13 amino acids, 14 amino acid, 15 amino acids, 16 amino acids, 17 amino acids, 18 amino acids, 19 amino acids, 20 amino acids, 21 amino acids, 22 amino acids, 23 amino acids, 24 amino acid, 25 amino acids, 26 amino acids, 27 amino acids, 28 amino acids, 29 amino acids, 30 amino acids, 31 amino acids, 32 amino acids, 33 amino acids, 34 amino acid, 35 amino acids, 36 amino acids, 37 amino acids, 38 amino acids, 39 amino acids, 40 amino acids, 41 amino acids, 42 amino acids, 43 amino acids, 44 amino acid, 45 amino acids, 46 amino acids, 47 amino acids, 48 amino acids, 49 amino acids, or 50 amino acids in length.

Charged and/or flexible linkers are particularly preferred.

Examples of flexible ABM linkers that can be used in the CD3 binding molecules (e.g., MBMs) include those disclosed by Chen et al., 2013, Adv Drug Deliv Rev. 65(10):1357-1369 and Klein et al., 2014, Protein Engineering, Design & Selection 27(10):325-330. A particularly useful flexible linker is (GGGGS)n (SEQ ID NO:24) also referred to as (G4S)n (SEQ ID NO: 24))). In some embodiments, n is any number between 1 and 10, i.e., 1, 2, 3, 4, 5, 6, 7, 8, 9, and 10, or any range bounded by any two of the foregoing numbers, e.g., 1 to 5, 2 to 5, 3 to 6, 2 to 4, 1 to 4, and so on and so forth.

Other examples of suitable ABM linkers for use in the CD3 binding molecules (e.g., MBMs) of the present disclosure are shown in Table 8 below:

TABLE 8
ABM Linker Sequences

| Linker | | |
|---|---|---|
| Name | Linker Sequence | SEQ ID NO: |
| L1 | ADAAP | SEQ ID NO: 25 |
| L2 | ADAAPTVSIFP | SEQ ID NO: 26 |
| L3 | ADAAPTVSIFPP | SEQ ID NO: 27 |
| L4 | AKTTAP | SEQ ID NO: 28 |
| L5 | AKTTAPSVYPLAP | SEQ ID NO: 29 |
| L6 | AKTTPKLEEGEFSEARV | SEQ ID NO: 30 |
| L7 | AKTTPKLGG | SEQ ID NO: 31 |
| L8 | AKTTPP | SEQ ID NO: 32 |
| L9 | AKTTPPSVTPLAP | SEQ ID NO: 33 |
| L10 | ASTKGP | SEQ ID NO: 34 |
| L11 | ASTKGPSVFPLAP | SEQ ID NO: 35 |
| L12 | ASTKGPSVFPLAPASTKGPSVFPLAP | SEQ ID NO: 36 |
| L13 | EGKSSGSGSESKST | SEQ ID NO: 37 |
| L14 | GEGESGEGESGEGES | SEQ ID NO: 38 |
| L15 | GEGESGEGESGEGESGEGES | SEQ ID NO: 39 |
| L16 | GEGGSGEGGSGEGGS | SEQ ID NO: 40 |
| L17 | GENKVEYAPALMALS | SEQ ID NO: 41 |
| L18 | GGEGSGGEGSGGEGS | SEQ ID NO: 42 |
| L19 | GGGESGGEGSGEGGS | SEQ ID NO: 43 |
| L20 | GGGESGGGESGGGES | SEQ ID NO: 44 |
| L21 | GGGGS | SEQ ID NO: 45 |
| L22 | GGGGSGGGGS | SEQ ID NO: 46 |
| L23 | GGGGSGGGGSGGGGS | SEQ ID NO: 47 |
| L24 | GGGGSGGGGSGGGGSGGGGS | SEQ ID NO: 48 |
| L25 | GGGKSGGGKSGGGKS | SEQ ID NO: 49 |
| L26 | GGGKSGGKGSGKGGS | SEQ ID NO: 50 |
| L27 | GGKGSGGKGSGGKGS | SEQ ID NO: 51 |
| L28 | GGSGG | SEQ ID NO: 52 |
| L29 | GGSGGGGSG | SEQ ID NO: 53 |
| L30 | GGSGGGGSGGGGS | SEQ ID NO: 54 |
| L31 | GHEAAAVMQVQYPAS | SEQ ID NO: 55 |
| L32 | GKGGSGKGGSGKGGS | SEQ ID NO: 56 |
| L33 | GKGKSGKGKSGKGKS | SEQ ID NO: 57 |
| L34 | GKGKSGKGKSGKGKSGKGKS | SEQ ID NO: 58 |
| L35 | GKPGSGKPGSGKPGS | SEQ ID NO: 59 |
| L36 | GKPGSGKPGSGKPGSGKPGS | SEQ ID NO: 60 |
| L37 | GPAKELTPLKEAKVS | SEQ ID NO: 61 |
| L38 | GSAGSAAGSGEF | SEQ ID NO: 62 |
| L39 | IRPRAIGGSKPRVA | SEQ ID NO: 63 |
| L40 | KESGSVSSEQLAQFRSLD | SEQ ID NO: 64 |
| L41 | KTTPKLEEGEFSEAR | SEQ ID NO: 65 |
| L42 | QPKAAP | SEQ ID NO: 66 |
| L43 | QPKAAPSVTLFPP | SEQ ID NO: 67 |
| L44 | RADAAAA(G4S)₄ | SEQ ID NO: 68 |
| L45 | RADAAAAGGPGS | SEQ ID NO: 69 |
| L46 | RADAAP | SEQ ID NO: 70 |
| L47 | RADAAPTVS | SEQ ID NO: 71 |
| L48 | SAKTTP | SEQ ID NO: 72 |
| L49 | SAKTTPKLEEGEFSEARV | SEQ ID NO: 73 |
| L50 | SAKTTPKLGG | SEQ ID NO: 74 |
| L51 | STAGDTHLGGEDFD | SEQ ID NO: 75 |
| L52 | TVAAP | SEQ ID NO: 76 |
| L53 | TVAAPSVFIFPP | SEQ ID NO: 77 |
| L54 | TVAAPSVFIFPPTVAAPSVFIFPP | SEQ ID NO: 78 |

In various aspects, the disclosure provides a CD3 binding molecule (e.g., MBM) which comprises one or more ABM linkers. Each of the ABM linkers can be range from 2 amino acids to 60 amino acids in length, preferably 4 to 30 amino acids, from 5 to 25 amino acids, from 10 to 25 amino acids or from 12 to 20 amino acids in length, optionally selected from Table 8 above. In particular embodiments, the CD3 binding molecule (e.g., MBM) comprises two, three, four, five or six ABM linkers. The ABM linkers can be on one, two, three, four or even more polypeptide chains of the CD3 binding molecule (e.g., MBM).

### 7.5. Bispecific Binding Molecule Configurations

Exemplary BBM configurations are shown in FIG. 1. FIG. 1A shows the components of the BBM configurations shown in FIGS. 1B-1AH. The scFv, Fab, scFab, non-immunoglobulin based ABM, and Fc domains each can have the characteristics described for these components in Sections 7.3 and 7.4. The components of the BBM configurations shown in FIG. 1 can be associated with each other by any of the means described in Sections 7.3 and 7.4 (e.g., by direct bonds, ABM linkers, disulfide bonds, Fc domains with modified with knob in hole interactions, etc.). The orientations and associations of the various components shown in FIG. 1 are merely exemplary; as will be appreciated by a skilled artisan, other orientations and associations can be suitable (e.g., as described in Sections 7.3 and 7.4).

BBMs are not limited to the configurations shown in FIG. 1. Other configurations that can be used are known to those skilled in the art. See, e.g., WO 2014/145806; WO 2017/124002; Liu et al., 2017, Front Immunol. 8:38; Brinkmann & Kontermann, 2017, mAbs 9:2, 182-212; US 2016/0355600; Klein et al., 2016, MAbs 8(6):1010-20; and US 2017/0145116.

7.5.1. Exemplary Bivalent BBMs

The BBMs can be bivalent, i.e., they have two antigen-binding domains, one or two of which binds CD3 (ABM1) and one of which binds a second target antigen (ABM2), e.g., CD2 or a TAA.

Exemplary bivalent BBM configurations are shown in FIGS. 1B-1F.

As depicted in FIGS. 1B-1D, a BBM can comprise two half antibodies, one comprising one ABM and the other comprising one ABM, the two halves paired through an Fc domain.

In the embodiment of FIG. 1B, the first (or left) half antibody comprises a Fab and an Fc region, and the second (or right) half antibody comprises a Fab and an Fc region. The first and second half antibodies are associated through the Fc regions forming an Fc domain.

In the embodiment of FIG. 1C, the first (or left) half antibody comprises a Fab and an Fc region, and the second (or right) half antibody comprises a scFv and an Fc region. The first and second half antibodies are associated through the Fc regions forming an Fc domain.

In the embodiment of FIG. 1D, the first (or left) half antibody comprises an scFv and an Fc region, and the second (or right) half antibody comprises an scFv and an Fc region. The first and second half antibodies are associated through the Fc regions forming an Fc domain.

As depicted in FIGS. 1E-1F, a bivalent BBM can comprise two ABMs attached to one Fc region of an Fc domain.

In the embodiment of FIG. 1E, the BBM comprises a Fab, a scFv and an Fc domain, where the scFv is located between the Fab and the Fc domain.

In the embodiment of FIG. 1F, (the “one-arm scFv-mAb” configuration) BBM comprises a Fab, a scFv and an Fc domain, where the Fab is located between the scFv and the Fc domain.

In the configuration shown in FIGS. 1B-1F, each of X and Y represent either ABM1 or ABM2, provided that the BBM comprises one ABM1 and one ABM2. Accordingly, the present disclosure provides a bivalent BBM as shown in any one of FIGS. 1B through 1F, where X is an ABM1 and Y is an ABM2 (this configuration of ABMs designated as “B1” for convenience). The present disclosure also provides a bivalent BBM as shown in any one of FIGS. 1B through 1F, where X is an ABM2 and Y is an ABM1 (this configuration of ABMs designated as “B2” for convenience).

7.5.2. Exemplary Trivalent BBMs

The BBMs can be trivalent, i.e., they have three antigen-binding domains, one or two of which binds CD3 (ABM1) and one or two of which binds a second target antigen (ABM2), e.g., CD2 or a TAA.

Exemplary trivalent BBM configurations are shown in FIGS. 1G-1Z.

As depicted in FIGS. 1G-1N, 1Q-1W, 1Y-1Z a BBM can comprise two half antibodies, one comprising two ABMs and the other comprising one ABM, the two halves paired through an Fc domain.

In the embodiment of FIG. 1G, the first (or left) half antibody comprises Fab and an Fc region, and the second (or right) half antibody comprises a scFv, a Fab, and an Fc region. The first and second half antibodies are associated through the Fc regions forming an Fc domain.

In the embodiment of FIG. 1H, the first (or left) half antibody comprises a Fab and an Fc region, and the second (or right) half antibody comprises a Fab, an scFv, and an Fc region. The first and second half antibodies are associated through the Fc regions forming an Fc domain.

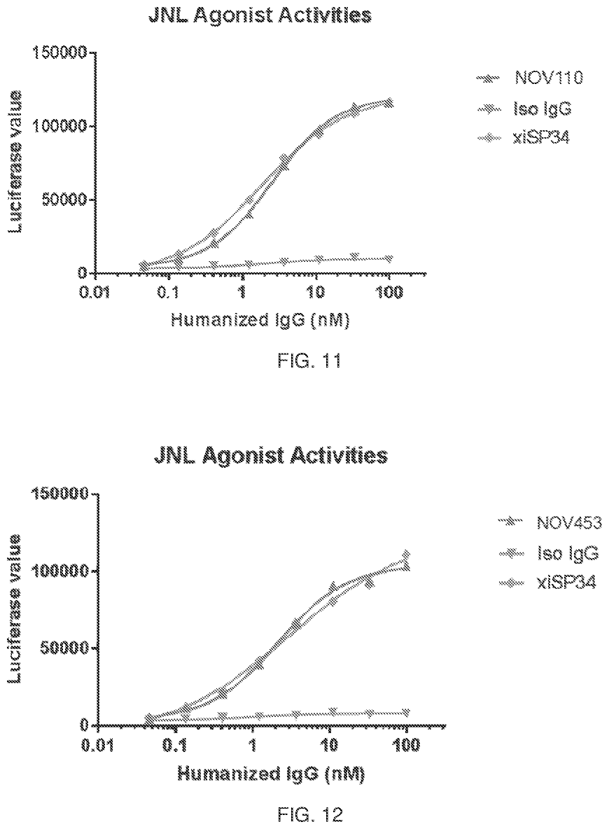

In the embodiment of FIG. 11, the first (or left) half antibody comprises an scFv and an Fc region, and the second (or right) half antibody comprises two Fabs and an Fc region. The first and second half antibodies are associated through the Fc regions forming an Fc domain.

In the embodiment of FIG. 1J, the first (or left) half antibody comprises two Fav and an Fc region, and the second (or right) half antibody comprises a Fab and an Fc region. The first and second half antibodies are associated through the Fc regions forming an Fc domain.

In the embodiment of FIG. 1K, the first (or left) half antibody comprises an scFv and an Fc region, and the second (or right) half antibody comprises two scFvs and an Fc region. The first and second half antibodies are associated through the Fc regions forming an Fc domain.

In the embodiment of FIG. 1L, the first (or left) half antibody comprises an scFv and an Fc region, and the second (or right) half antibody comprises an scFv, a Fab, and an Fc region. The first and second half antibodies are associated through the Fc regions forming an Fc domain.

In the embodiment of FIG. 1M, the first (or left) half antibody comprises a scFv and an Fc region, and the second (or right) half antibody comprises a Fab, a scFv and an Fc region. The first and second half antibodies are associated through the Fc regions forming an Fc domain.

In the embodiment of FIG. 1N, the first (or left) half antibody comprises a diabody-type binding domain and an Fc region, and the second (or right) half antibody comprises a Fab and an Fc region. The first and second half antibodies are associated through the Fc regions forming an Fc domain.

In the embodiment of FIG. 1Q, the first (or left) half antibody comprises a Fab and an Fc region, and the second (or right) half antibody comprises a Fab, an Fc region, and an scFv. The first and second half antibodies are associated through the Fc regions forming an Fc domain.

In the embodiment of FIG. 1R, the first (or left) half antibody comprises a scFv and an Fc region, and the second (or right) half antibody comprises a Fab, an Fc region, and an scFv. The first and second half antibodies are associated through the Fc regions forming an Fc domain.

In the embodiment of FIG. 1S, the first (or left) half antibody comprises an scFv and an Fc region, and the second (or right) half antibody comprises an scFv, an Fc region, and a second scFv. The first and second half antibodies are associated through the Fc regions forming an Fc domain.

In the embodiment of FIG. 1T, the first (or left) half antibody comprises an scFv, an Fc region, and a Fab, and the second (or right) half antibody comprises a Fab and an Fc region. The first and second half antibodies are associated through the Fc regions forming an Fc domain.

In the embodiment of FIG. 1U, the first (or left) half antibody comprises two Fab and an Fc region, and the second (or right) half antibody comprises a non-immunoglobulin based ABM and an Fc region. The first and second half antibodies are associated through the Fc regions forming an Fc domain.

In the embodiment of FIG. 1V, the first (or left) half antibody comprises a Fab, an scFv, and an Fc region, and the second (or right) half antibody comprises a non-immunoglobulin based ABM and an Fc region. The first and second half antibodies are associated through the Fc regions forming an Fc domain.

In the embodiment of FIG. 1W, the first (or left) half antibody comprises a Fab and an Fc region, and the second (or right) half antibody comprises a scFv, a non-immunoglobulin based ABM, and an Fc region. The first and second half antibodies are associated through the Fc regions forming an Fc domain.

In the embodiment of FIG. 1Y, the first (or left) half antibody comprises an scFv and an Fc region, and the second (or right) half antibody comprises a Fab, an scFv and an Fc region. The first and second half antibodies are associated through the Fc regions forming an Fc domain.

In the embodiment of FIG. 1Z, the first (or left) half antibody comprises a Fab, an Fc region, and a scFab, and the second (or right) half antibody comprises a Fab and an Fc region. The first and second half antibodies are associated through the Fc regions forming an Fc domain.

Alternatively, as depicted in FIGS. 1O and 1P, trivalent a BBM can comprise two half antibodies, each comprising one complete ABM (a Fab in FIGS. 1O and 1P) and a portion of another ABM (one a VH, the other a VL). The two half antibodies are paired through an Fc domain, whereupon the VH and the VL associate to form a complete antigen-binding Fv domain.

The BBM can be a single chain, as shown in FIG. 1X. The BBM of FIG. 1X comprises three scFv domains connected through linkers.

In the configuration shown in FIGS. 1G-1Z, each of X, Y and A represent either an ABM1 or ABM2, provided that the BBM comprises at least ABM1 and at least one ABM2. Thus, the trivalent MBMs will include one or two ABM1s and one or two ABM2s. In some embodiments, a trivalent BBM comprises two ABM1s and one ABM2. In other embodiments, a trivalent BBM comprises one ABM1 and two ABM2s.

Accordingly, in the present disclosure provides a trivalent BBM as shown in any one of FIGS. 1G through 1Z, where X is an ABM1, Y is an ABM1 and A is an ABM2 (this configuration of ABMs designated as “T1” for convenience).

The disclosure further provides a trivalent BBM as shown in any one of FIGS. 1G through 1Z, where X is an ABM1, Y is an ABM2 and A is an ABM1 (this configuration of ABMs designated as “T2” for convenience).

The disclosure further provides a trivalent BBM as shown in any one of FIGS. 1G through 1Z, where X is an ABM2, Y is an ABM1 and A is an ABM1 (this configuration of ABMs designated as “T3” for convenience).

The disclosure further provides a trivalent BBM as shown in any one of FIGS. 1G through 1Z, where X is an ABM1, Y is an ABM2 and A is an ABM2 (this configuration of ABMs designated as “T4” for convenience).

The disclosure further provides a trivalent BBM as shown in any one of FIGS. 1G through 1Z, where X is an ABM2, Y is an ABM1 and A is an ABM2 (this configuration of ABMs designated as “T5” for convenience).

The disclosure further provides a trivalent BBM as shown in any one of FIGS. 1G through 1Z, where X is an ABM2, Y is an ABM2 and A is an ABM1 (this configuration of ABMs designated as “T6” for convenience).

7.5.3. Exemplary Tetravalent BBMs

The BBMs can be tetravalent, i.e., they have four antigen-binding domains, one, two, or three of which binds CD2 (ABM1) and one, two, or three of which binds a second target antigen (ABM2), e.g., CD2 or a TAA.

Exemplary tetravalent BBM configurations are shown in FIGS. 1AA-1AH.

As depicted in FIGS. 1AA-1AH, a tetravalent BBM can comprise two half antibodies, each comprising two complete ABMs, the two halves paired through an Fc domain.

In the embodiment of FIG. 1AA, the first (or left) half antibody comprises a Fab, an Fc region, and an scFv, and the second (or right) half antibody comprises a Fab, an Fc region, and an scFv. The first and second half antibodies are associated through the Fc regions forming an Fc domain.

In the embodiment of FIG. 1AB, the first (or left) half antibody comprises a Fab, an scFv, and an Fc region, and the second (or right) half antibody comprises a Fab, an scFv, and an Fc region. The first and second half antibodies are associated through the Fc regions forming an Fc domain.

In the embodiment of FIG. 1AC, the first (or left) half antibody comprises an scFv, a Fab, and an Fc region, and the second (or right) half antibody comprises an scFv, a Fab, and an Fc region. The first and second half antibodies are associated through the Fc regions forming an Fc domain.

In the embodiment of FIG. 1AD, the first (or left) half antibody comprises a Fab, an Fc region, and a second Fab, and the second (or right) half antibody comprises a Fab, an Fc region, and a second Fab. The first and second half antibodies are associated through the Fc regions forming an Fc domain.

In the embodiment of FIG. 1AE, the first (or left) half antibody comprises an scFv, a second scFv, and an Fc region, and the second (or right) half antibody comprises an scFv, a second scFv, and an Fc region. The first and second half antibodies are associated through the Fc regions forming an Fc domain.

In the embodiment of FIG. 1AF, the first (or left) half antibody comprises a Fab, an scFv, and an Fc region, and the second (or right) half antibody comprises a Fab, an scFv, and an Fc region. The first and second half antibodies are associated through the Fc regions forming an Fc domain.

In the embodiment of FIG. 1AG, the first (or left) half antibody comprises a Fab, an Fc region, and an scFv, and the second (or right) half antibody comprises a scFv, an Fc region, and a Fab. The first and second half antibodies are associated through the Fc regions forming an Fc domain.

In the embodiment of FIG. 1AH, the first (or left) half antibody comprises a scFv, an Fc region, and an Fab, and the second (or right) half antibody comprises a scFv, an Fc region, and a Fab. The first and second half antibodies are associated through the Fc regions forming an Fc domain.

In the configuration shown in FIGS. 1AA-1AH, each of X, Y, A, and B represent ABM1 or ABM2, although not necessarily in that order, and provided that the BBM comprises at least one ABM1 and at least one ABM2. Thus, the tetravalent ABMs will include one, two, or three ABM1s and one, two, or ABM2s. In some embodiments, a tetravalent BBM comprises three ABM1s and one ABM2. In other embodiments, a tetravalent BBM comprises two ABM1s two ABM2s. In yet other embodiments, a tetravalent BBM comprises one ABM1 and three ABM2s.

Accordingly, in the present disclosure provides a tetravalent BBM as shown in any one of FIGS. 1AA-1AH, where X is an ABM1 and each of Y, A, and B are ABM2s (this configuration of ABMs designated as “Tv 1” for convenience).

The disclosure further provides a tetravalent BBM as shown in any one of FIGS. 1AA-1AH, where Y is an ABM1 and each of X, A, and B are ABM2s (this configuration of ABMs designated as “Tv 2” for convenience).

The disclosure further provides a tetravalent BBM as shown in any one of FIGS. 1AA-1AH, where A is an ABM1 and each of X, Y, and B are ABM2s (this configuration of ABMs designated as “Tv 3” for convenience).

The disclosure further provides a tetravalent BBM as shown in any one of FIGS. 1AA-1AH, where B is an ABM1 and each of X, Y, and A are ABM2s (this configuration of ABMs designated as “Tv 4” for convenience).

The disclosure further provides a tetravalent BBM as shown in any one of FIGS. 1AA-1AH, where X and Y are both ABM1s and both of A and B are ABM2s (this configuration of ABMs designated as “Tv 5” for convenience).

The disclosure further provides a tetravalent BBM as shown in any one of FIGS. 1AA-1AH, where X and A are both ABM1s and both of Y and B are ABM2s (this configuration of ABMs designated as “Tv 6” for convenience).

The disclosure further provides a tetravalent BBM as shown in any one of FIGS. 1AA-1AH, where X and B are both ABM1s and both of Y and A are ABM2s (this configuration of ABMs designated as “Tv 7” for convenience).

The disclosure further provides a tetravalent BBM as shown in any one of FIGS. 1AA-1AH, where Y and A are both ABM1s and both of X and B are ABM2s (this configuration of ABMs designated as “Tv 8” for convenience).

The disclosure further provides a tetravalent BBM as shown in any one of FIGS. 1AA-1AH, where Y and B are both ABM1s and both of X and A are ABM2s (this configuration of ABMs designated as “Tv 9” for convenience).

The disclosure further provides a tetravalent BBM as shown in any one of FIGS. 1AA-1AH, where A and B are both ABM1s and both of X and Y are ABM2s (this configuration of ABMs designated as “Tv 10” for convenience).

The disclosure further provides a tetravalent BBM as shown in any one of FIGS. 1AA-1AH, where each of X, Y, and A is an ABM1 and B is an ABM2 (this configuration of ABMs designated as “Tv 11” for convenience).

The disclosure further provides a tetravalent BBM as shown in any one of FIGS. 1AA-1AH, where each of X, Y, and B is an ABM1 and A is an ABM2 (this configuration of ABMs designated as “Tv 12” for convenience).

The disclosure further provides a tetravalent BBM as shown in any one of FIGS. 1AA-1AH, where each of X, A, and B is an ABM1 and Y is an ABM2 (this configuration of ABMs designated as “Tv 13” for convenience).

The disclosure further provides a tetravalent BBM as shown in any one of FIGS. 1AA-1AH, where each of Y, A, and B is an ABM1 and X is an ABM2 (this configuration of ABMs designated as “Tv 14” for convenience).

### 7.6. Trispecific Binding Molecule Configurations

Exemplary TBM configurations are shown in FIG. 2. FIG. 2A shows the components of the TBM configurations shown in FIGS. 2B-1V. The scFv, Fab, non-immunoglobulin based ABM, and Fc each can have the characteristics described for these components in Sections 7.3 and 7.4. The components of the TBM configurations shown in FIG. 2 can be associated with each other by any of the means described in Sections 7.3 and 7.4 (e.g., by direct bonds, ABM linkers, disulfide bonds, Fc domains with modified with knob in hole interactions, etc.). The orientations and associations of the various components shown in FIG. 2 are merely exemplary; as will be appreciated by a skilled artisan, other orientations and associations can be suitable (e.g., as described in Sections 7.3 and 7.4).

TBMs are not limited to the configurations shown in FIG. 2. Other configurations that can be used are known to those skilled in the art. See, e.g., WO 2014/145806; WO 2017/124002; Liu et al., 2017, Front Immunol. 8:38; Brinkmann & Kontermann, 2017, mAbs 9:2, 182-212; US 2016/0355600; Klein et al., 2016, MAbs 8(6):1010-20; and US 2017/0145116.

7.6.1. Exemplary Trivalent TBMs

The TBMs can be trivalent, i.e., they have three antigen-binding domains, one of which binds CD3, one of which binds a TAA, and one of which binds either CD2 or a second TAA.

Exemplary trivalent TBM configurations are shown in FIGS. 2B through 2P.

As depicted in FIGS. 2B-2K and 2N-2P, a TBM can comprise two half antibodies, one comprising two ABMs and the other comprising one ABM, the two halves paired through an Fc domain.

In the embodiment of FIG. 2B, the first (or left) half antibody comprises an scFv and an Fc region, and the second (or right) half antibody comprises a Fab, an scFv and an Fc region. The first and second half antibodies are associated through the Fc regions forming an Fc domain.

In the embodiment of FIG. 2C, the first (or left) half antibody comprises two Fab and an Fc region, and the second (or right) half antibody comprises a Fab and an Fc region. The first and second half antibodies are associated through the Fc regions forming an Fc domain.

In the embodiment of FIG. 2D, the first (or left) half antibody comprises a Fab, an scFv and an Fc region, and the second (or right) half antibody comprises a Fab and an Fc region. The first and second half antibodies are associated through the Fc regions forming an Fc domain.

In the embodiment of FIG. 2E, the first (or left) half antibody comprises an scFv and an Fc region, and the second (or right) half antibody comprises two Fab and an Fc region. The first and second half antibodies are associated through the Fc regions forming an Fc domain.

In the embodiment of FIG. 2F, the first (or left) half antibody comprises an scFv, an Fc region, and a Fab, and the second (or right) half antibody comprises a Fab and an Fc region. The first and second half antibodies are associated through the Fc regions forming an Fc domain.

In the embodiment of FIG. 2G, the first (or left) half antibody comprises an scFv and an Fc region, and the second (or right) half antibody comprises a Fab an Fc region, and an scFV. The first and second half antibodies are associated through the Fc regions forming an Fc domain.

In the embodiment of FIG. 2H, the first (or left) half antibody comprises two Fab and an Fc region, and the second (or right) half antibody comprises a non-immunoglobulin based ABM and an Fc region. The first and second half antibodies are associated through the Fc regions forming an Fc domain.

In the embodiment of FIG. 2I, the first (or left) half antibody comprises a Fab, an scFv, and an Fc region, and the second (or right) half antibody comprises a non-immunoglobulin based ABM and an Fc region. The first and second half antibodies are associated through the Fc regions forming an Fc domain.

In the embodiment of FIG. 2J, the first (or left) half antibody comprises a Fab and an Fc region, and the second (or right) half antibody comprises an scFv, a non-immunoglobulin based ABM and an Fc region. The first and second half antibodies are associated through the Fc regions forming an Fc domain.

In the embodiment of FIG. 2K, the first (or left) half antibody comprises an scFv and an Fc region, and the second (or right) half antibody comprises an scFv, an Fc region, and a second scFv. The first and second half antibodies are associated through the Fc regions forming an Fc domain.

In the embodiment of FIG. 2N, the first (or left) half antibody comprises a Fab, an Fc region, and an scFv, and the second (or right) half antibody comprises a Fab, and an Fc region. The first and second half antibodies are associated through the Fc regions forming an Fc domain.

In the embodiment of FIG. 2O, the first (or left) half antibody comprises a Fab, an Fc region, and a scFab, and the second (or right) half antibody comprises a Fab and an Fc region. The first and second half antibodies are associated through the Fc regions forming an Fc domain.

In the embodiment of FIG. 2P, the first (or left) half antibody comprises a Fab, a non-immunoglobulin based ABM, and an Fc region, and the second (or right) half antibody comprises a scFv and an Fc region. The first and second half antibodies are associated through the Fc regions forming an Fc domain.

Alternatively, as depicted in FIG. 2L, trivalent a TBM can comprise two half antibodies, each comprising one complete ABM and a portion of another ABM (one a VH, the other a VL). The two half antibodies are paired through an Fc domain, whereupon the VH and the VL associate to form a complete antigen-binding Fv domain.

The TBM can be a single chain, as shown in FIG. 2M. The TBM of FIG. 2M comprises three scFv domains connected through linkers.

In each of the configurations shown in FIGS. 2B-2P, each of the domains designated X, Y, and Z represents an ABM1, ABM2, or ABM3, although not necessarily in that order. In other words, X can be ABM1, ABM2, or ABM3, Y can be ABM1, ABM2, or ABM3, and Z can be ABM1, ABM2, or ABM3, provided that the TBM comprises one ABM1, one ABM2, and one ABM3.

Accordingly, in the present disclosure provides a trivalent TBM as shown in any one of FIGS. 2B through 2P, where X is an ABM1, Y is an ABM3 and Z is an ABM2 (this configuration of ABMs designated as “T1” for convenience).

The present disclosure also provides a trivalent TBM as shown in any one of FIGS. 2B through 2P, where X is an ABM1, Y is an ABM2, and Z is an ABM3 (this configuration of ABMs designated as “T2” for convenience).

The present disclosure further provides a trivalent TBM as shown in any one of FIGS. 2B through 2P, where X is an ABM3, Y is an ABM1, and Z is an ABM2 (this configuration of ABMs designated as “T3” for convenience).

The present disclosure yet further provides a trivalent TBM as shown in any one of FIGS. 2B through 2P, where X is an ABM3, Y is an ABM2, and Z is an ABM1 (this configuration of ABMs designated as “T4” for convenience).

The present disclosure yet further provides a trivalent TBM as shown in any one of FIGS. 2B through 2P, where X is an ABM2, Y is an ABM1, and Z is an ABM3 (this configuration of ABMs designated as “T5” for convenience).

The present disclosure yet further provides a trivalent TBM as shown in any one of FIGS. 2B through 2P, where X is an ABM2, Y is an ABM3, and Z is an ABM1 (this configuration of ABMs designated as “T6” for convenience).

7.6.2. Exemplary Tetravalent TBMs

The TBMs can be tetravalent, i.e., they have four antigen-binding domains, one or two of which binds CD3, one or two of which binds a TAA, and one or two of which binds CD2 or a second TAA.

Exemplary tetravalent TBM configurations are shown in FIGS. 2Q-2S.

As depicted in FIGS. 2Q-2S, a tetravalent TBM can comprise two half antibodies, each comprising two complete ABMs, the two halves paired through an Fc domain.

In the embodiment of FIG. 2Q, the first (or left) half antibody comprises a Fab, an Fc region, and a second Fab, and the second (or right) half antibody comprises a Fab, an Fc region, and a second Fab. The first and second half antibodies are associated through the Fc regions forming an Fc domain.

In the embodiment of FIG. 2R, the first (or left) half antibody comprises a Fab, an Fc region, and an scFv, and the second (or right) half antibody comprises a Fab, an Fc region, and an scFv. The first and second half antibodies are associated through the Fc regions forming an Fc domain.

In the embodiment of FIG. 2S, the first (or left) half antibody comprises a Fab, an Fc region, and an scFv, and the second (or right) half antibody comprises an scFv, an Fc region, and a Fab. The first and second half antibodies are associated through the Fc regions forming an Fc domain.

In the configuration shown in FIGS. 2Q-2S, each of X, Y, Z, and A represent an ABM1, an ABM2, or an ABM3, although not necessarily in that order, and provided that the TBM comprises at least one ABM1, at least one ABM2, and at least one ABM3. Thus, the tetravalent ABMs will include two ABMs against one of CD3, a TAA, and CD2 or a second TAA. In some cases, a tetravalent TBM has two CD3 ABMs.

Accordingly, the present disclosure provides tetravalent TBMs as shown in any one of FIGS. 2Q-2S, where X, Y, Z, and A are ABMs directed to CD3, a TAA and CD2 or a second TAA, as shown in Table 9.

TABLE 9
ABM Permutations in Tetravalent TBMs

| Tetravalent Configuration | X | Y | Z | A |
|---|---|---|---|---|
| Tv 1 | CD3 | CD3 | CD2 or TAA2 | TAA1 |
| Tv 2 | CD3 | CD3 | TAA1 | CD2 or TAA2 |
| Tv 3 | CD3 | CD2 or TAA2 | CD3 | TAA1 |
| Tv 4 | CD3 | TAA1 | CD3 | CD2 or TAA2 |
| Tv 5 | CD3 | CD2 or TAA2 | TAA1 | CD3 |
| Tv 6 | CD3 | TAA1 | CD2 or TAA2 | CD3 |
| Tv 7 | CD2 or TAA2 | CD3 | CD3 | TAA1 |
| Tv 8 | TAA1 | CD3 | CD3 | CD2 or TAA2 |
| Tv 9 | CD2 or TAA2 | CD3 | TAA1 | CD3 |
| Tv 10 | TAA1 | CD3 | CD2 or TAA2 | CD3 |
| Tv 11 | CD2 or TAA2 | TAA1 | CD3 | CD3 |
| Tv 12 | TAA1 | CD2 or TAA2 | CD3 | CD3 |
| Tv 13 | CD3 | CD2 or TAA2 | TAA1 | TAA1 |
| Tv 14 | CD3 | TAA1 | CD2 or TAA2 | TAA1 |
| Tv 15 | CD3 | TAA1 | TAA1 | CD2 or TAA2 |
| Tv 16 | CD2 or TAA2 | CD3 | TAA1 | TAA1 |
| Tv 17 | TAA1 | CD3 | CD2 or TAA2 | TAA1 |
| Tv 18 | TAA1 | CD3 | TAA1 | CD2 or TAA2 |
| Tv 19 | CD2 or TAA2 | TAA1 | CD3 | TAA1 |
| Tv 20 | TAA1 | CD2 or TAA2 | CD3 | TAA1 |
| Tv 21 | TAA1 | TAA1 | CD3 | CD2 or TAA2 |
| Tv 22 | CD2 or TAA2 | TAA1 | TAA1 | CD3 |
| Tv 23 | TAA1 | CD2 or TAA2 | TAA1 | CD3 |
| Tv 24 | TAA1 | TAA1 | CD2 or TAA2 | CD3 |

7.6.3. Exemplary Pentavalent TBMs

The TBMs can be pentavalent, i.e., they have five antigen-binding domains, one, two, or three of which binds CD3, one, two, or three of which binds a TAA, and one, two, or three of which binds CD2 or a second TAA.

An exemplary pentavalent TBM configuration is shown in FIG. 2T.

As depicted in FIG. 2T, a pentavalent TBM can comprise two half antibodies, one of which comprises two complete ABMs and the other of which comprises one complete ABM, the two halves paired through an Fc domain.

In the embodiment of FIG. 2T, the first (or left) half antibody comprises a Fab, an scFv, and an Fc region, and the second (or right) half antibody comprises a Fab, an Fc region, and an scFv. The first and second half antibodies are associated through the Fc regions forming an Fc domain.

In the configuration shown in FIG. 2T, each of X, Y, Z, A, and B represent an ABM1, an ABM2, or an ABM3, although not necessarily in that order, and provided that the TBM comprises at least one ABM1, one ABM2, and one ABM3. Thus, the pentavalent TBMs can include two ABMs against two of CD3, a TAA, and CD2 or a second TAA, or three ABMs against one of CD3, a TAA, and CD2 or a second TAA. In some cases, a pentavalent TBM has two or three CD3 ABMs. In some embodiments, a pentavalent TBM has three ABM1s, one ABM2 and one ABM3.

Accordingly, the present disclosure provides a pentavalent TBM as shown in FIG. 2T, where X, Y, Z, A, and B are ABMs directed to CD3, a TAA, and CD2 or a second TAA as shown in Table 10.

TABLE 10
ABM Permutations in Pentavalent TBMs

| Pentavalent | | | | | |
|---|---|---|---|---|---|
| Configuration | X | Y | Z | A | B |
| Pv 1 | CD3 | CD3 | CD3 | CD2 or TAA2 | TAA1 |
| Pv 2 | CD3 | CD3 | CD3 | TAA1 | CD2 or TAA2 |
| Pv 3 | CD3 | CD3 | CD2 or TAA2 | CD3 | TAA1 |
| Pv 4 | CD3 | CD3 | TAA1 | CD3 | CD2 or TAA2 |
| Pv 5 | CD3 | CD3 | CD2 or TAA2 | TAA1 | CD3 |
| Pv 6 | CD3 | CD3 | TAA1 | CD2 or TAA2 | CD3 |
| Pv 7 | CD3 | CD2 or TAA2 | CD3 | CD3 | TAA1 |
| Pv 8 | CD3 | TAA1 | CD3 | CD3 | CD2 or TAA2 |
| Pv 9 | CD3 | CD2 or TAA2 | CD3 | TAA1 | CD3 |
| Pv 10 | CD3 | TAA1 | CD3 | CD2 or TAA2 | CD3 |
| Pv 11 | CD3 | CD2 or TAA2 | TAA1 | CD3 | CD3 |
| Pv 12 | CD3 | TAA1 | CD2 or TAA2 | CD3 | CD3 |
| Pv 13 | CD2 or TAA2 | CD3 | CD3 | CD3 | TAA1 |
| Pv 14 | TAA1 | CD3 | CD3 | CD3 | CD2 or TAA2 |
| Pv 15 | CD2 or TAA2 | CD3 | CD3 | TAA1 | CD3 |
| Pv 16 | TAA1 | CD3 | CD3 | CD2 or TAA2 | CD3 |
| Pv 17 | CD2 or TAA2 | CD3 | TAA1 | CD3 | CD3 |
| Pv 18 | TAA1 | CD3 | CD2 or TAA2 | CD3 | CD3 |
| Pv 19 | CD2 or TAA2 | TAA1 | CD3 | CD3 | CD3 |
| Pv 20 | TAA1 | CD2 or TAA2 | CD3 | CD3 | CD3 |
| Pv 21 | CD3 | CD3 | CD2 or TAA2 | CD2 or TAA2 | TAA1 |
| Pv 22 | CD3 | CD3 | CD2 or TAA2 | TAA1 | CD2 or TAA2 |
| Pv 23 | CD3 | CD3 | TAA1 | CD2 or TAA2 | CD2 or TAA2 |
| Pv 24 | CD3 | CD2 or TAA2 | CD3 | CD2 or TAA2 | TAA1 |
| Pv 25 | CD3 | CD2 or TAA2 | CD3 | TAA1 | CD2 or TAA2 |
| Pv 26 | CD3 | TAA1 | CD3 | CD2 or TAA2 | CD2 or TAA2 |
| Pv 27 | CD3 | CD2 or TAA2 | CD2 or TAA2 | CD3 | TAA1 |
| Pv 28 | CD3 | CD2 or TAA2 | TAA1 | CD3 | CD2 or TAA2 |
| Pv 29 | CD3 | TAA1 | CD2 or TAA2 | CD3 | CD2 or TAA2 |
| Pv 30 | CD3 | CD2 or TAA2 | CD2 or TAA2 | TAA1 | CD3 |
| Pv 31 | CD3 | CD2 or TAA2 | TAA1 | CD2 or TAA2 | CD3 |
| Pv 32 | CD3 | TAA1 | CD2 or TAA2 | CD2 or TAA2 | CD3 |
| Pv 33 | CD2 or TAA2 | CD3 | CD3 | CD2 or TAA2 | TAA1 |
| Pv 34 | CD2 or TAA2 | CD3 | CD3 | TAA1 | CD2 or TAA2 |
| Pv 35 | TAA1 | CD3 | CD3 | CD2 or TAA2 | CD2 or TAA2 |
| Pv 36 | CD2 or TAA2 | CD3 | CD2 or TAA2 | CD3 | TAA1 |
| Pv 37 | CD2 or TAA2 | CD3 | TAA1 | CD3 | CD2 or TAA2 |
| Pv 38 | TAA1 | CD3 | CD2 or TAA2 | CD3 | CD2 or TAA2 |
| Pv 39 | CD2 or TAA2 | CD3 | CD2 or TAA2 | TAA1 | CD3 |
| Pv 40 | CD2 or TAA2 | CD3 | TAA1 | CD2 or TAA2 | CD3 |
| Pv 41 | TAA1 | CD3 | CD2 or TAA2 | CD2 or TAA2 | CD3 |
| Pv 42 | CD2 or TAA2 | CD2 or TAA2 | CD3 | CD3 | TAA1 |
| Pv 43 | CD2 or TAA2 | TAA1 | CD3 | CD3 | CD2 or TAA2 |
| Pv 44 | TAA1 | CD2 or TAA2 | CD3 | CD3 | CD2 or TAA2 |
| Pv 45 | CD2 or TAA2 | CD2 or TAA2 | CD3 | TAA1 | CD3 |
| Pv 46 | CD2 or TAA2 | TAA1 | CD3 | CD2 or TAA2 | CD3 |
| Pv 47 | TAA1 | CD2 or TAA2 | CD3 | CD2 or TAA2 | CD3 |
| Pv 48 | CD2 or TAA2 | CD2 or TAA2 | TAA1 | CD3 | CD3 |
| Pv 49 | CD2 or TAA2 | TAA1 | CD2 or TAA2 | CD3 | CD3 |
| Pv 50 | TAA1 | CD2 or TAA2 | CD2 or TAA2 | CD3 | CD3 |
| Pv 51 | CD3 | CD3 | CD2 or TAA2 | TAA1 | TAA1 |
| Pv 52 | CD3 | CD3 | TAA1 | CD2 or TAA2 | TAA1 |
| Pv 53 | CD3 | CD3 | TAA1 | TAA1 | CD2 or TAA2 |
| Pv 54 | CD3 | CD2 or TAA2 | CD3 | TAA1 | TAA1 |
| Pv 55 | CD3 | TAA1 | CD3 | CD2 or TAA2 | TAA1 |
| Pv 56 | CD3 | TAA1 | CD3 | TAA1 | CD2 or TAA2 |
| Pv 57 | CD3 | CD2 or TAA2 | TAA1 | CD3 | TAA1 |
| Pv 58 | CD3 | TAA1 | CD2 or TAA2 | CD3 | TAA1 |
| Pv 59 | CD3 | TAA1 | TAA1 | CD3 | CD2 or TAA2 |
| Pv 60 | CD3 | CD2 or TAA2 | TAA1 | TAA1 | CD3 |
| Pv 61 | CD3 | TAA1 | CD2 or TAA2 | TAA1 | CD3 |
| Pv 62 | CD3 | TAA1 | TAA1 | CD2 or TAA2 | CD3 |
| Pv 63 | CD2 or TAA2 | CD3 | CD3 | TAA1 | TAA1 |
| Pv 64 | TAA1 | CD3 | CD3 | CD2 or TAA2 | TAA1 |
| Pv 65 | TAA1 | CD3 | CD3 | TAA1 | CD2 or TAA2 |
| Pv 66 | CD2 or TAA2 | CD3 | TAA1 | CD3 | TAA1 |
| Pv 67 | TAA1 | CD3 | CD2 or TAA2 | CD3 | TAA1 |
| Pv 68 | TAA1 | CD3 | TAA1 | CD3 | CD2 or TAA2 |
| Pv 69 | CD2 or TAA2 | CD3 | TAA1 | TAA1 | CD3 |
| Pv 70 | TAA1 | CD3 | CD2 or TAA2 | TAA1 | CD3 |
| Pv 71 | TAA1 | CD3 | TAA1 | CD2 or TAA2 | CD3 |
| Pv 72 | CD2 or TAA2 | TAA1 | CD3 | CD3 | TAA1 |
| Pv 73 | TAA1 | CD2 or TAA2 | CD3 | CD3 | TAA1 |
| Pv 74 | TAA1 | TAA1 | CD3 | CD3 | CD2 or TAA2 |
| Pv 75 | CD2 or TAA2 | TAA1 | CD3 | TAA1 | CD3 |
| Pv 76 | TAA1 | CD2 or TAA2 | CD3 | TAA1 | CD3 |
| Pv 77 | TAA1 | TAA1 | CD3 | CD2 or TAA2 | CD3 |
| Pv 78 | CD2 or TAA2 | TAA1 | TAA1 | CD3 | CD3 |
| Pv 79 | TAA1 | CD2 or TAA2 | TAA1 | CD3 | CD3 |
| Pv 80 | TAA1 | TAA1 | CD2 or TAA2 | CD3 | CD3 |
| Pv 81 | CD3 | CD2 or TAA2 | TAA1 | TAA1 | TAA1 |
| Pv 82 | CD3 | TAA1 | CD2 or TAA2 | TAA1 | TAA1 |
| Pv 83 | CD3 | TAA1 | TAA1 | CD2 or TAA2 | TAA1 |
| Pv 84 | CD3 | TAA1 | TAA1 | TAA1 | CD2 or TAA2 |
| Pv 85 | CD2 or TAA2 | CD3 | TAA1 | TAA1 | TAA1 |
| Pv 86 | TAA1 | CD3 | CD2 or TAA2 | TAA1 | TAA1 |
| Pv 87 | TAA1 | CD3 | TAA1 | CD2 or TAA2 | TAA1 |
| Pv 88 | TAA1 | CD3 | TAA1 | TAA1 | CD2 or TAA2 |
| Pv 89 | CD2 or TAA2 | TAA1 | CD3 | TAA1 | TAA1 |
| Pv 90 | TAA1 | CD2 or TAA2 | CD3 | TAA1 | TAA1 |
| Pv 91 | TAA1 | TAA1 | CD3 | CD2 or TAA2 | TAA1 |
| Pv 92 | TAA1 | TAA1 | CD3 | TAA1 | CD2 or TAA2 |
| Pv 93 | CD2 or TAA2 | TAA1 | TAA1 | CD3 | TAA1 |
| Pv 94 | TAA1 | CD2 or TAA2 | TAA1 | CD3 | TAA1 |
| Pv 95 | TAA1 | TAA1 | CD2 or TAA2 | CD3 | TAA1 |
| Pv 96 | TAA1 | TAA1 | TAA1 | CD3 | CD2 or TAA2 |
| Pv 97 | CD2 or TAA2 | TAA1 | TAA1 | TAA1 | CD3 |
| Pv 98 | TAA1 | CD2 or TAA2 | TAA1 | TAA1 | CD3 |
| Pv 99 | TAA1 | TAA1 | CD2 or TAA2 | TAA1 | CD3 |
| Pv 100 | TAA1 | TAA1 | TAA1 | CD2 or TAA2 | CD3 |

7.6.4. Exemplary Hexavalent TBMs

The TBMs can be hexavalent, i.e., they have six antigen-binding domains, one, two, three, or four of which binds CD3, one, two, three, or four of which binds a TAA, and one, two, three, or four of which binds CD2 or a second TAA.

Exemplary hexavalent TBM configurations are shown in FIGS. 2U-2V.

As depicted in FIGS. 2U-2V, a pentavalent TBM can comprise two half antibodies, one of which comprises two complete ABMs and the other of which comprises one complete ABM, the two halves paired through an Fc domain.

In the embodiment of FIG. 2U, the first (or left) half antibody comprises a Fab, a second Fab, an Fc region, and an scFv, and the second (or right) half antibody comprises a Fab, a second Fab, an Fc region, and an scFv. The first and second half antibodies are associated through the Fc regions forming an Fc domain.

In the embodiment of FIG. 2V, the first (or left) half antibody comprises a first Fv, a second Fv, a third Fv, and an Fc region, and the second (or right) half antibody comprises a first Fv, a second Fv, a third Fv, and an Fc region. The first and second half antibodies are associated through the Fc regions forming an Fc domain.

In the configuration shown in FIGS. 2U-2V, each of X, Y, Z, A, B, and C represent an ABM1, an ABM2, or an ABM3, although not necessarily in that order, and provided that the TBM comprises at least one ABM1, one ABM2, and one ABM3. Thus, the hexavalent TBMs can include (i) two ABMs against each of CD3, a TAA, and CD2 or a second TAA, (ii) three ABMs against one of CD3, a TAA, and CD2 or a second TAA, or (iii) four ABMs against one of CD3, a TAA, and CD2 or a second TAA. For example, a hexavalent ABM can include three ABMs against CD3, two ABMs against a TAA and one ABM against CD2 or a second TAA. As another example, a hexavalent ABM can include three ABMs against CD3, one ABM against a TAA and two ABMs against CD2 or a second TAA. In some cases, a hexavalent TBM has two, three, our four CD3 ABMs. In some embodiments, a hexavalent TBM has three CD3 ABMs. In other embodiments, a hexavalent TBM has four CD3 ABMs.

Accordingly, in the present disclosure provides hexavalent TBMs as shown in any one of FIGS. 2U-2V, where X, Y, Z, A, B, and C are ABMs directed to CD3, a TAA, and CD2 or a second TAA, as shown in Table 11.

TABLE 11
ABM Permutations in Hexavalent TBMs

| Hexavalent | | | | | | |
|---|---|---|---|---|---|---|
| Configuration | X | Y | Z | A | B | C |
| Hv 1 | CD3 | CD3 | CD3 | CD3 | CD2 or TAA2 | TAA1 |
| Hv 2 | CD3 | CD3 | CD3 | CD3 | TAA1 | CD2 or TAA2 |
| Hv 3 | CD3 | CD3 | CD3 | CD2 or TAA2 | CD3 | TAA1 |
| Hv 4 | CD3 | CD3 | CD3 | TAA1 | CD3 | CD2 or TAA2 |
| Hv 5 | CD3 | CD3 | CD3 | CD2 or TAA2 | TAA1 | CD3 |
| Hv 6 | CD3 | CD3 | CD3 | TAA1 | CD2 or TAA2 | CD3 |
| Hv 7 | CD3 | CD3 | CD2 or TAA2 | CD3 | CD3 | TAA1 |
| Hv 8 | CD3 | CD3 | TAA1 | CD3 | CD3 | CD2 or TAA2 |
| Hv 9 | CD3 | CD3 | CD2 or TAA2 | CD3 | TAA1 | CD3 |
| Hv 10 | CD3 | CD3 | TAA1 | CD3 | CD2 or TAA2 | CD3 |
| Hv 11 | CD3 | CD3 | CD2 or TAA2 | TAA1 | CD3 | CD3 |
| Hv 12 | CD3 | CD3 | TAA1 | CD2 or TAA2 | CD3 | CD3 |
| Hv 13 | CD3 | CD2 or TAA2 | CD3 | CD3 | CD3 | TAA1 |
| Hv 14 | CD3 | TAA1 | CD3 | CD3 | CD3 | CD2 or TAA2 |
| Hv 15 | CD3 | CD2 or TAA2 | CD3 | CD3 | TAA1 | CD3 |
| Hv 16 | CD3 | TAA1 | CD3 | CD3 | CD2 or TAA2 | CD3 |
| Hv 17 | CD3 | CD2 or TAA2 | CD3 | TAA1 | CD3 | CD3 |
| Hv 18 | CD3 | TAA1 | CD3 | CD2 or TAA2 | CD3 | CD3 |
| Hv 19 | CD3 | CD2 or TAA2 | TAA1 | CD3 | CD3 | CD3 |
| Hv 20 | CD3 | TAA1 | CD2 or TAA2 | CD3 | CD3 | CD3 |
| Hv 21 | CD2 or TAA2 | CD3 | CD3 | CD3 | CD3 | TAA1 |
| Hv 22 | TAA1 | CD3 | CD3 | CD3 | CD3 | CD2 or TAA2 |
| Hv 23 | CD2 or TAA2 | CD3 | CD3 | CD3 | TAA1 | CD3 |
| Hv 24 | TAA1 | CD3 | CD3 | CD3 | CD2 or TAA2 | CD3 |
| Hv 25 | CD2 or TAA2 | CD3 | CD3 | TAA1 | CD3 | CD3 |
| Hv 26 | TAA1 | CD3 | CD3 | CD2 or TAA2 | CD3 | CD3 |
| Hv 27 | CD2 or TAA2 | CD3 | TAA1 | CD3 | CD3 | CD3 |
| Hv 28 | TAA1 | CD3 | CD2 or TAA2 | CD3 | CD3 | CD3 |
| Hv 29 | CD2 or TAA2 | TAA1 | CD3 | CD3 | CD3 | CD3 |
| Hv 30 | TAA1 | CD2 or TAA2 | CD3 | CD3 | CD3 | CD3 |
| Hv 31 | CD3 | CD3 | CD3 | CD2 or TAA2 | CD2 or TAA2 | TAA1 |
| Hv 32 | CD3 | CD3 | CD3 | CD2 or TAA2 | TAA1 | CD2 or TAA2 |
| Hv 33 | CD3 | CD3 | CD3 | TAA1 | CD2 or TAA2 | CD2 or TAA2 |
| Hv 34 | CD3 | CD3 | CD2 or TAA2 | CD3 | CD2 or TAA2 | TAA1 |
| Hv 35 | CD3 | CD3 | CD2 or TAA2 | CD3 | TAA1 | CD2 or TAA2 |
| Hv 36 | CD3 | CD3 | TAA1 | CD3 | CD2 or TAA2 | CD2 or TAA2 |
| Hv 37 | CD3 | CD3 | CD2 or TAA2 | CD2 or TAA2 | CD3 | TAA1 |
| Hv 38 | CD3 | CD3 | CD2 or TAA2 | TAA1 | CD3 | CD2 or TAA2 |
| Hv 39 | CD3 | CD3 | TAA1 | CD2 or TAA2 | CD3 | CD2 or TAA2 |
| Hv 40 | CD3 | CD3 | CD2 or TAA2 | CD2 or TAA2 | TAA1 | CD3 |
| Hv 41 | CD3 | CD3 | CD2 or TAA2 | TAA1 | CD2 or TAA2 | CD3 |
| Hv 42 | CD3 | CD3 | TAA1 | CD2 or TAA2 | CD2 or TAA2 | CD3 |
| Hv 43 | CD3 | CD2 or TAA2 | CD3 | CD3 | CD2 or TAA2 | TAA1 |
| Hv 44 | CD3 | CD2 or TAA2 | CD3 | CD3 | TAA1 | CD2 or TAA2 |
| Hv 45 | CD3 | TAA1 | CD3 | CD3 | CD2 or TAA2 | CD2 or TAA2 |
| Hv 46 | CD3 | CD2 or TAA2 | CD3 | CD2 or TAA2 | CD3 | TAA1 |
| Hv 47 | CD3 | CD2 or TAA2 | CD3 | TAA1 | CD3 | CD2 or TAA2 |
| Hv 48 | CD3 | TAA1 | CD3 | CD2 or TAA2 | CD3 | CD2 or TAA2 |
| Hv 49 | CD3 | CD2 or TAA2 | CD3 | CD2 or TAA2 | TAA1 | CD3 |
| Hv 50 | CD3 | CD2 or TAA2 | CD3 | TAA1 | CD2 or TAA2 | CD3 |
| Hv 51 | CD3 | TAA1 | CD3 | CD2 or TAA2 | CD2 or TAA2 | CD3 |
| Hv 52 | CD3 | CD2 or TAA2 | CD2 or TAA2 | CD3 | CD3 | TAA1 |
| Hv 53 | CD3 | CD2 or TAA2 | TAA1 | CD3 | CD3 | CD2 or TAA2 |
| Hv 54 | CD3 | TAA1 | CD2 or TAA2 | CD3 | CD3 | CD2 or TAA2 |
| Hv 55 | CD3 | CD2 or TAA2 | CD2 or TAA2 | CD3 | TAA1 | CD3 |
| Hv 56 | CD3 | CD2 or TAA2 | TAA1 | CD3 | CD2 or TAA2 | CD3 |
| Hv 57 | CD3 | TAA1 | CD2 or TAA2 | CD3 | CD2 or TAA2 | CD3 |
| Hv 58 | CD3 | CD2 or TAA2 | CD2 or TAA2 | TAA1 | CD3 | CD3 |
| Hv 59 | CD3 | CD2 or TAA2 | TAA1 | CD2 or TAA2 | CD3 | CD3 |
| Hv 60 | CD3 | TAA1 | CD2 or TAA2 | CD2 or TAA2 | CD3 | CD3 |
| Hv 61 | CD2 or TAA2 | CD3 | CD3 | CD3 | CD2 or TAA2 | TAA1 |
| Hv 62 | CD2 or TAA2 | CD3 | CD3 | CD3 | TAA1 | CD2 or TAA2 |
| Hv 63 | TAA1 | CD3 | CD3 | CD3 | CD2 or TAA2 | CD2 or TAA2 |
| Hv 64 | CD2 or TAA2 | CD3 | CD3 | CD2 or TAA2 | CD3 | TAA1 |
| Hv 65 | CD2 or TAA2 | CD3 | CD3 | TAA1 | CD3 | CD2 or TAA2 |
| Hv 66 | TAA1 | CD3 | CD3 | CD2 or TAA2 | CD3 | CD2 or TAA2 |
| Hv 67 | CD2 or TAA2 | CD3 | CD3 | CD2 or TAA2 | TAA1 | CD3 |
| Hv 68 | CD2 or TAA2 | CD3 | CD3 | TAA1 | CD2 or TAA2 | CD3 |
| Hv 69 | TAA1 | CD3 | CD3 | CD2 or TAA2 | CD2 or TAA2 | CD3 |
| Hv 70 | CD2 or TAA2 | CD3 | CD2 or TAA2 | CD3 | CD3 | TAA1 |
| Hv 71 | CD2 or TAA2 | CD3 | TAA1 | CD3 | CD3 | CD2 or TAA2 |
| Hv 72 | TAA1 | CD3 | CD2 or TAA2 | CD3 | CD3 | CD2 or TAA2 |
| Hv 73 | CD2 or TAA2 | CD3 | CD2 or TAA2 | CD3 | TAA1 | CD3 |
| Hv 74 | CD2 or TAA2 | CD3 | TAA1 | CD3 | CD2 or TAA2 | CD3 |
| Hv 75 | TAA1 | CD3 | CD2 or TAA2 | CD3 | CD2 or TAA2 | CD3 |
| Hv 76 | CD2 or TAA2 | CD3 | CD2 or TAA2 | TAA1 | CD3 | CD3 |
| Hv 77 | CD2 or TAA2 | CD3 | TAA1 | CD2 or TAA2 | CD3 | CD3 |
| Hv 78 | TAA1 | CD3 | CD2 or TAA2 | CD2 or TAA2 | CD3 | CD3 |
| Hv 79 | CD2 or TAA2 | CD2 or TAA2 | CD3 | CD3 | CD3 | TAA1 |
| Hv 80 | CD2 or TAA2 | TAA1 | CD3 | CD3 | CD3 | CD2 or TAA2 |
| Hv 81 | TAA1 | CD2 or TAA2 | CD3 | CD3 | CD3 | CD2 or TAA2 |
| Hv 82 | CD2 or TAA2 | CD2 or TAA2 | CD3 | CD3 | TAA1 | CD3 |
| Hv 83 | CD2 or TAA2 | TAA1 | CD3 | CD3 | CD2 or TAA2 | CD3 |
| Hv 84 | TAA1 | CD2 or TAA2 | CD3 | CD3 | CD2 or TAA2 | CD3 |
| Hv 85 | CD2 or TAA2 | CD2 or TAA2 | CD3 | TAA1 | CD3 | CD3 |
| Hv 86 | CD2 or TAA2 | TAA1 | CD3 | CD2 or TAA2 | CD3 | CD3 |
| Hv 87 | TAA1 | CD2 or TAA2 | CD3 | CD2 or TAA2 | CD3 | CD3 |
| Hv 88 | CD2 or TAA2 | CD2 or TAA2 | TAA1 | CD3 | CD3 | CD3 |
| Hv 89 | CD2 or TAA2 | TAA1 | CD2 or TAA2 | CD3 | CD3 | CD3 |
| Hv 90 | TAA1 | CD2 or TAA2 | CD2 or TAA2 | CD3 | CD3 | CD3 |
| Hv 91 | CD3 | CD3 | CD3 | CD2 or TAA2 | TAA1 | TAA1 |
| Hv 92 | CD3 | CD3 | CD3 | TAA1 | CD2 or TAA2 | TAA1 |
| Hv 93 | CD3 | CD3 | CD3 | TAA1 | TAA1 | CD2 or TAA2 |
| Hv 94 | CD3 | CD3 | CD2 or TAA2 | CD3 | TAA1 | TAA1 |
| Hv 95 | CD3 | CD3 | TAA1 | CD3 | CD2 or TAA2 | TAA1 |
| Hv 96 | CD3 | CD3 | TAA1 | CD3 | TAA1 | CD2 or TAA2 |
| Hv 97 | CD3 | CD3 | CD2 or TAA2 | TAA1 | CD3 | TAA1 |
| Hv 98 | CD3 | CD3 | TAA1 | CD2 or TAA2 | CD3 | TAA1 |
| Hv 99 | CD3 | CD3 | TAA1 | TAA1 | CD3 | CD2 or TAA2 |
| Hv 100 | CD3 | CD3 | CD2 or TAA2 | TAA1 | TAA1 | CD3 |
| Hv 101 | CD3 | CD3 | TAA1 | CD2 or TAA2 | TAA1 | CD3 |
| Hv 102 | CD3 | CD3 | TAA1 | TAA1 | CD2 or TAA2 | CD3 |
| Hv 103 | CD3 | CD2 or TAA2 | CD3 | CD3 | TAA1 | TAA1 |
| Hv 104 | CD3 | TAA1 | CD3 | CD3 | CD2 or TAA2 | TAA1 |
| Hv 105 | CD3 | TAA1 | CD3 | CD3 | TAA1 | CD2 or TAA2 |
| Hv 106 | CD3 | CD2 or TAA2 | CD3 | TAA1 | CD3 | TAA1 |
| Hv 107 | CD3 | TAA1 | CD3 | CD2 or TAA2 | CD3 | TAA1 |
| Hv 108 | CD3 | TAA1 | CD3 | TAA1 | CD3 | CD2 or TAA2 |
| Hv 109 | CD3 | CD2 or TAA2 | CD3 | TAA1 | TAA1 | CD3 |
| Hv 110 | CD3 | TAA1 | CD3 | CD2 or TAA2 | TAA1 | CD3 |
| Hv 111 | CD3 | TAA1 | CD3 | TAA1 | CD2 or TAA2 | CD3 |
| Hv 112 | CD3 | CD2 or TAA2 | TAA1 | CD3 | CD3 | TAA1 |
| Hv 113 | CD3 | TAA1 | CD2 or TAA2 | CD3 | CD3 | TAA1 |
| Hv 114 | CD3 | TAA1 | TAA1 | CD3 | CD3 | CD2 or TAA2 |
| Hv 115 | CD3 | CD2 or TAA2 | TAA1 | CD3 | TAA1 | CD3 |
| Hv 116 | CD3 | TAA1 | CD2 or TAA2 | CD3 | TAA1 | CD3 |
| Hv 117 | CD3 | TAA1 | TAA1 | CD3 | CD2 or TAA2 | CD3 |
| Hv 118 | CD3 | CD2 or TAA2 | TAA1 | TAA1 | CD3 | CD3 |
| Hv 119 | CD3 | TAA1 | CD2 or TAA2 | TAA1 | CD3 | CD3 |
| Hv 120 | CD3 | TAA1 | TAA1 | CD2 or TAA2 | CD3 | CD3 |
| Hv 121 | CD2 or TAA2 | CD3 | CD3 | CD3 | TAA1 | TAA1 |
| Hv 122 | TAA1 | CD3 | CD3 | CD3 | CD2 or TAA2 | TAA1 |
| Hv 123 | TAA1 | CD3 | CD3 | CD3 | TAA1 | CD2 or TAA2 |
| Hv 124 | CD2 or TAA2 | CD3 | CD3 | TAA1 | CD3 | TAA1 |
| Hv 125 | TAA1 | CD3 | CD3 | CD2 or TAA2 | CD3 | TAA1 |
| Hv 126 | TAA1 | CD3 | CD3 | TAA1 | CD3 | CD2 or TAA2 |
| Hv 127 | CD2 or TAA2 | CD3 | CD3 | TAA1 | TAA1 | CD3 |
| Hv 128 | TAA1 | CD3 | CD3 | CD2 or TAA2 | TAA1 | CD3 |
| Hv 129 | TAA1 | CD3 | CD3 | TAA1 | CD2 or TAA2 | CD3 |
| Hv 130 | CD2 or TAA2 | CD3 | TAA1 | CD3 | CD3 | TAA1 |
| Hv 131 | TAA1 | CD3 | CD2 or TAA2 | CD3 | CD3 | TAA1 |
| Hv 132 | TAA1 | CD3 | TAA1 | CD3 | CD3 | CD2 or TAA2 |
| Hv 133 | CD2 or TAA2 | CD3 | TAA1 | CD3 | TAA1 | CD3 |
| Hv 134 | TAA1 | CD3 | CD2 or TAA2 | CD3 | TAA1 | CD3 |
| Hv 135 | TAA1 | CD3 | TAA1 | CD3 | CD2 or TAA2 | CD3 |
| Hv 136 | CD2 or TAA2 | CD3 | TAA1 | TAA1 | CD3 | CD3 |
| Hv 137 | TAA1 | CD3 | CD2 or TAA2 | TAA1 | CD3 | CD3 |
| Hv 138 | TAA1 | CD3 | TAA1 | CD2 or TAA2 | CD3 | CD3 |
| Hv 139 | CD2 or TAA2 | TAA1 | CD3 | CD3 | CD3 | TAA1 |
| Hv 140 | TAA1 | CD2 or TAA2 | CD3 | CD3 | CD3 | TAA1 |
| Hv 141 | TAA1 | TAA1 | CD3 | CD3 | CD3 | CD2 or TAA2 |
| Hv 142 | CD2 or TAA2 | TAA1 | CD3 | CD3 | TAA1 | CD3 |
| Hv 143 | TAA1 | CD2 or TAA2 | CD3 | CD3 | TAA1 | CD3 |
| Hv 144 | TAA1 | TAA1 | CD3 | CD3 | CD2 or TAA2 | CD3 |
| Hv 145 | CD2 or TAA2 | TAA1 | CD3 | TAA1 | CD3 | CD3 |
| Hv 146 | TAA1 | CD2 or TAA2 | CD3 | TAA1 | CD3 | CD3 |
| Hv 147 | TAA1 | TAA1 | CD3 | CD2 or TAA2 | CD3 | CD3 |
| Hv 148 | CD2 or TAA2 | TAA1 | TAA1 | CD3 | CD3 | CD3 |
| Hv 149 | TAA1 | CD2 or TAA2 | TAA1 | CD3 | CD3 | CD3 |
| Hv 150 | TAA1 | TAA1 | CD2 or TAA2 | CD3 | CD3 | CD3 |
| Hv 151 | CD3 | CD3 | CD2 or TAA2 | CD2 or TAA2 | TAA1 | TAA1 |
| Hv 152 | CD3 | CD3 | CD2 or TAA2 | TAA1 | CD2 or TAA2 | TAA1 |
| Hv 153 | CD3 | CD3 | CD2 or TAA2 | TAA1 | TAA1 | CD2 or TAA2 |
| Hv 154 | CD3 | CD3 | TAA1 | CD2 or TAA2 | CD2 or TAA2 | TAA1 |
| Hv 155 | CD3 | CD3 | TAA1 | CD2 or TAA2 | TAA1 | CD2 or TAA2 |
| Hv 156 | CD3 | CD3 | TAA1 | TAA1 | CD2 or TAA2 | CD2 or TAA2 |
| Hv 157 | CD3 | CD2 or TAA2 | CD3 | CD2 or TAA2 | TAA1 | TAA1 |
| Hv 158 | CD3 | CD2 or TAA2 | CD3 | TAA1 | CD2 or TAA2 | TAA1 |
| Hv 159 | CD3 | CD2 or TAA2 | CD3 | TAA1 | TAA1 | CD2 or TAA2 |
| Hv 160 | CD3 | TAA1 | CD3 | CD2 or TAA2 | CD2 or TAA2 | TAA1 |
| Hv 161 | CD3 | TAA1 | CD3 | CD2 or TAA2 | TAA1 | CD2 or TAA2 |
| Hv 162 | CD3 | TAA1 | CD3 | TAA1 | CD2 or TAA2 | CD2 or TAA2 |
| Hv 163 | CD3 | CD2 or TAA2 | CD2 or TAA2 | CD3 | TAA1 | TAA1 |
| Hv 164 | CD3 | CD2 or TAA2 | TAA1 | CD3 | CD2 or TAA2 | TAA1 |
| Hv 165 | CD3 | CD2 or TAA2 | TAA1 | CD3 | TAA1 | CD2 or TAA2 |
| Hv 166 | CD3 | TAA1 | CD2 or TAA2 | CD3 | CD2 or TAA2 | TAA1 |
| Hv 167 | CD3 | TAA1 | CD2 or TAA2 | CD3 | TAA1 | CD2 or TAA2 |
| Hv 168 | CD3 | TAA1 | TAA1 | CD3 | CD2 or TAA2 | CD2 or TAA2 |
| Hv 169 | CD3 | CD2 or TAA2 | CD2 or TAA2 | TAA1 | CD3 | TAA1 |
| Hv 170 | CD3 | CD2 or TAA2 | TAA1 | CD2 or TAA2 | CD3 | TAA1 |
| Hv 171 | CD3 | CD2 or TAA2 | TAA1 | TAA1 | CD3 | CD2 or TAA2 |
| Hv 172 | CD3 | TAA1 | CD2 or TAA2 | CD2 or TAA2 | CD3 | TAA1 |
| Hv 173 | CD3 | TAA1 | CD2 or TAA2 | TAA1 | CD3 | CD2 or TAA2 |
| Hv 174 | CD3 | TAA1 | TAA1 | CD2 or TAA2 | CD3 | CD2 or TAA2 |
| Hv 175 | CD3 | CD2 or TAA2 | CD2 or TAA2 | TAA1 | TAA1 | CD3 |
| Hv 176 | CD3 | CD2 or TAA2 | TAA1 | CD2 or TAA2 | TAA1 | CD3 |
| Hv 177 | CD3 | CD2 or TAA2 | TAA1 | TAA1 | CD2 or TAA2 | CD3 |
| Hv 178 | CD3 | TAA1 | CD2 or TAA2 | CD2 or TAA2 | TAA1 | CD3 |
| Hv 179 | CD3 | TAA1 | CD2 or TAA2 | TAA1 | CD2 or TAA2 | CD3 |
| Hv 180 | CD3 | TAA1 | TAA1 | CD2 or TAA2 | CD2 or TAA2 | CD3 |
| Hv 181 | CD2 or TAA2 | CD3 | CD3 | CD2 or TAA2 | TAA1 | TAA1 |
| Hv 182 | CD2 or TAA2 | CD3 | CD3 | TAA1 | CD2 or TAA2 | TAA1 |
| Hv 183 | CD2 or TAA2 | CD3 | CD3 | TAA1 | TAA1 | CD2 or TAA2 |
| Hv 184 | TAA1 | CD3 | CD3 | CD2 or TAA2 | CD2 or TAA2 | TAA1 |
| Hv 185 | TAA1 | CD3 | CD3 | CD2 or TAA2 | TAA1 | CD2 or TAA2 |
| Hv 186 | TAA1 | CD3 | CD3 | TAA1 | CD2 or TAA2 | CD2 or TAA2 |
| Hv 187 | CD2 or TAA2 | CD3 | CD2 or TAA2 | CD3 | TAA1 | TAA1 |
| Hv 188 | CD2 or TAA2 | CD3 | TAA1 | CD3 | CD2 or TAA2 | TAA1 |
| Hv 189 | CD2 or TAA2 | CD3 | TAA1 | CD3 | TAA1 | CD2 or TAA2 |
| Hv 190 | TAA1 | CD3 | CD2 or TAA2 | CD3 | CD2 or TAA2 | TAA1 |
| Hv 191 | TAA1 | CD3 | CD2 or TAA2 | CD3 | TAA1 | CD2 or TAA2 |
| Hv 192 | TAA1 | CD3 | TAA1 | CD3 | CD2 or TAA2 | CD2 or TAA2 |
| Hv 193 | CD2 or TAA2 | CD3 | CD2 or TAA2 | TAA1 | CD3 | TAA1 |
| Hv 194 | CD2 or TAA2 | CD3 | TAA1 | CD2 or TAA2 | CD3 | TAA1 |
| Hv 195 | CD2 or TAA2 | CD3 | TAA1 | TAA1 | CD3 | CD2 or TAA2 |
| Hv 196 | TAA1 | CD3 | CD2 or TAA2 | CD2 or TAA2 | CD3 | TAA1 |
| Hv 197 | TAA1 | CD3 | CD2 or TAA2 | TAA1 | CD3 | CD2 or TAA2 |
| Hv 198 | TAA1 | CD3 | TAA1 | CD2 or TAA2 | CD3 | CD2 or TAA2 |
| Hv 199 | CD2 or TAA2 | CD3 | CD2 or TAA2 | TAA1 | TAA1 | CD3 |
| Hv 200 | CD2 or TAA2 | CD3 | TAA1 | CD2 or TAA2 | TAA1 | CD3 |
| Hv 201 | CD2 or TAA2 | CD3 | TAA1 | TAA1 | CD2 or TAA2 | CD3 |
| Hv 202 | TAA1 | CD3 | CD2 or TAA2 | CD2 or TAA2 | TAA1 | CD3 |
| Hv 203 | TAA1 | CD3 | CD2 or TAA2 | TAA1 | CD2 or TAA2 | CD3 |
| Hv 204 | TAA1 | CD3 | TAA1 | CD2 or TAA2 | CD2 or TAA2 | CD3 |
| Hv 205 | CD2 or TAA2 | CD2 or TAA2 | CD3 | CD3 | TAA1 | TAA1 |
| Hv 206 | CD2 or TAA2 | TAA1 | CD3 | CD3 | CD2 or TAA2 | TAA1 |
| Hv 207 | CD2 or TAA2 | TAA1 | CD3 | CD3 | TAA1 | CD2 or TAA2 |
| Hv 208 | TAA1 | CD2 or TAA2 | CD3 | CD3 | CD2 or TAA2 | TAA1 |
| Hv 209 | TAA1 | CD2 or TAA2 | CD3 | CD3 | TAA1 | CD2 or TAA2 |
| Hv 210 | TAA1 | TAA1 | CD3 | CD3 | CD2 or TAA2 | CD2 or TAA2 |
| Hv 211 | CD2 or TAA2 | CD2 or TAA2 | CD3 | TAA1 | CD3 | TAA1 |
| Hv 212 | CD2 or TAA2 | TAA1 | CD3 | CD2 or TAA2 | CD3 | TAA1 |
| Hv 213 | CD2 or TAA2 | TAA1 | CD3 | TAA1 | CD3 | CD2 or TAA2 |
| Hv 214 | TAA1 | CD2 or TAA2 | CD3 | CD2 or TAA2 | CD3 | TAA1 |
| Hv 215 | TAA1 | CD2 or TAA2 | CD3 | TAA1 | CD3 | CD2 or TAA2 |
| Hv 216 | TAA1 | TAA1 | CD3 | CD2 or TAA2 | CD3 | CD2 or TAA2 |
| Hv 217 | CD2 or TAA2 | CD2 or TAA2 | CD3 | TAA1 | TAA1 | CD3 |
| Hv 218 | CD2 or TAA2 | TAA1 | CD3 | CD2 or TAA2 | TAA1 | CD3 |
| Hv 219 | CD2 or TAA2 | TAA1 | CD3 | TAA1 | CD2 or TAA2 | CD3 |
| Hv 220 | TAA1 | CD2 or TAA2 | CD3 | CD2 or TAA2 | TAA1 | CD3 |
| Hv 221 | TAA1 | CD2 or TAA2 | CD3 | TAA1 | CD2 or TAA2 | CD3 |
| Hv 222 | TAA1 | TAA1 | CD3 | CD2 or TAA2 | CD2 or TAA2 | CD3 |
| Hv 223 | CD2 or TAA2 | CD2 or TAA2 | TAA1 | CD3 | CD3 | TAA1 |
| Hv 224 | CD2 or TAA2 | TAA1 | CD2 or TAA2 | CD3 | CD3 | TAA1 |
| Hv 225 | CD2 or TAA2 | TAA1 | TAA1 | CD3 | CD3 | CD2 or TAA2 |
| Hv 226 | TAA1 | CD2 or TAA2 | CD2 or TAA2 | CD3 | CD3 | TAA1 |
| Hv 227 | TAA1 | CD2 or TAA2 | TAA1 | CD3 | CD3 | CD2 or TAA2 |
| Hv 228 | TAA1 | TAA1 | CD2 or TAA2 | CD3 | CD3 | CD2 or TAA2 |
| Hv 229 | CD2 or TAA2 | CD2 or TAA2 | TAA1 | CD3 | TAA1 | CD3 |
| Hv 230 | CD2 or TAA2 | TAA1 | CD2 or TAA2 | CD3 | TAA1 | CD3 |
| Hv 231 | CD2 or TAA2 | TAA1 | TAA1 | CD3 | CD2 or TAA2 | CD3 |
| Hv 232 | TAA1 | CD2 or TAA2 | CD2 or TAA2 | CD3 | TAA1 | CD3 |
| Hv 233 | TAA1 | CD2 or TAA2 | TAA1 | CD3 | CD2 or TAA2 | CD3 |
| Hv 234 | TAA1 | TAA1 | CD2 or TAA2 | CD3 | CD2 or TAA2 | CD3 |
| Hv 235 | CD2 or TAA2 | CD2 or TAA2 | TAA1 | TAA1 | CD3 | CD3 |
| Hv 236 | CD2 or TAA2 | TAA1 | CD2 or TAA2 | TAA1 | CD3 | CD3 |
| Hv 237 | CD2 or TAA2 | TAA1 | TAA1 | CD2 or TAA2 | CD3 | CD3 |
| Hv 238 | TAA1 | CD2 or TAA2 | CD2 or TAA2 | TAA1 | CD3 | CD3 |
| Hv 239 | TAA1 | CD2 or TAA2 | TAA1 | CD2 or TAA2 | CD3 | CD3 |
| Hv 240 | TAA1 | TAA1 | CD2 or TAA2 | CD2 or TAA2 | CD3 | CD3 |
| Hv 241 | CD3 | CD3 | CD2 or TAA2 | TAA1 | TAA1 | TAA1 |
| Hv 242 | CD3 | CD3 | TAA1 | CD2 or TAA2 | TAA1 | TAA1 |
| Hv 243 | CD3 | CD3 | TAA1 | TAA1 | CD2 or TAA2 | TAA1 |
| Hv 244 | CD3 | CD3 | TAA1 | TAA1 | TAA1 | CD2 or TAA2 |
| Hv 245 | CD3 | CD2 or TAA2 | CD3 | TAA1 | TAA1 | TAA1 |
| Hv 246 | CD3 | TAA1 | CD3 | CD2 or TAA2 | TAA1 | TAA1 |
| Hv 247 | CD3 | TAA1 | CD3 | TAA1 | CD2 or TAA2 | TAA1 |
| Hv 248 | CD3 | TAA1 | CD3 | TAA1 | TAA1 | CD2 or TAA2 |
| Hv 249 | CD3 | CD2 or TAA2 | TAA1 | CD3 | TAA1 | TAA1 |
| Hv 250 | CD3 | TAA1 | CD2 or TAA2 | CD3 | TAA1 | TAA1 |
| Hv 251 | CD3 | TAA1 | TAA1 | CD3 | CD2 or TAA2 | TAA1 |
| Hv 252 | CD3 | TAA1 | TAA1 | CD3 | TAA1 | CD2 or TAA2 |
| Hv 253 | CD3 | CD2 or TAA2 | TAA1 | TAA1 | CD3 | TAA1 |
| Hv 254 | CD3 | TAA1 | CD2 or TAA2 | TAA1 | CD3 | TAA1 |
| Hv 255 | CD3 | TAA1 | TAA1 | CD2 or TAA2 | CD3 | TAA1 |
| Hv 256 | CD3 | TAA1 | TAA1 | TAA1 | CD3 | CD2 or TAA2 |
| Hv 257 | CD3 | CD2 or TAA2 | TAA1 | TAA1 | TAA1 | CD3 |
| Hv 258 | CD3 | TAA1 | CD2 or TAA2 | TAA1 | TAA1 | CD3 |
| Hv 259 | CD3 | TAA1 | TAA1 | CD2 or TAA2 | TAA1 | CD3 |
| Hv 260 | CD3 | TAA1 | TAA1 | TAA1 | CD2 or TAA2 | CD3 |
| Hv 261 | CD2 or TAA2 | CD3 | CD3 | TAA1 | TAA1 | TAA1 |
| Hv 262 | TAA1 | CD3 | CD3 | CD2 or TAA2 | TAA1 | TAA1 |
| Hv 263 | TAA1 | CD3 | CD3 | TAA1 | CD2 or TAA2 | TAA1 |
| Hv 264 | TAA1 | CD3 | CD3 | TAA1 | TAA1 | CD2 or TAA2 |
| Hv 265 | CD2 or TAA2 | CD3 | TAA1 | CD3 | TAA1 | TAA1 |
| Hv 266 | TAA1 | CD3 | CD2 or TAA2 | CD3 | TAA1 | TAA1 |
| Hv 267 | TAA1 | CD3 | TAA1 | CD3 | CD2 or TAA2 | TAA1 |
| Hv 268 | TAA1 | CD3 | TAA1 | CD3 | TAA1 | CD2 or TAA2 |
| Hv 269 | CD2 or TAA2 | CD3 | TAA1 | TAA1 | CD3 | TAA1 |
| Hv 270 | TAA1 | CD3 | CD2 or TAA2 | TAA1 | CD3 | TAA1 |
| Hv 271 | TAA1 | CD3 | TAA1 | CD2 or TAA2 | CD3 | TAA1 |
| Hv 272 | TAA1 | CD3 | TAA1 | TAA1 | CD3 | CD2 or TAA2 |
| Hv 273 | CD2 or TAA2 | CD3 | TAA1 | TAA1 | TAA1 | CD3 |
| Hv 274 | TAA1 | CD3 | CD2 or TAA2 | TAA1 | TAA1 | CD3 |
| Hv 275 | TAA1 | CD3 | TAA1 | CD2 or TAA2 | TAA1 | CD3 |
| Hv 276 | TAA1 | CD3 | TAA1 | TAA1 | CD2 or TAA2 | CD3 |
| Hv 277 | CD2 or TAA2 | TAA1 | CD3 | CD3 | TAA1 | TAA1 |
| Hv 278 | TAA1 | CD2 or TAA2 | CD3 | CD3 | TAA1 | TAA1 |
| Hv 279 | TAA1 | TAA1 | CD3 | CD3 | CD2 or TAA2 | TAA1 |
| Hv 280 | TAA1 | TAA1 | CD3 | CD3 | TAA1 | CD2 or TAA2 |
| Hv 281 | CD2 or TAA2 | TAA1 | CD3 | TAA1 | CD3 | TAA1 |
| Hv 282 | TAA1 | CD2 or TAA2 | CD3 | TAA1 | CD3 | TAA1 |
| Hv 283 | TAA1 | TAA1 | CD3 | CD2 or TAA2 | CD3 | TAA1 |
| Hv 284 | TAA1 | TAA1 | CD3 | TAA1 | CD3 | CD2 or TAA2 |
| Hv 285 | CD2 or TAA2 | TAA1 | CD3 | TAA1 | TAA1 | CD3 |
| Hv 286 | TAA1 | CD2 or TAA2 | CD3 | TAA1 | TAA1 | CD3 |
| Hv 287 | TAA1 | TAA1 | CD3 | CD2 or TAA2 | TAA1 | CD3 |
| Hv 288 | TAA1 | TAA1 | CD3 | TAA1 | CD2 or TAA2 | CD3 |
| Hv 289 | CD2 or TAA2 | TAA1 | TAA1 | CD3 | CD3 | TAA1 |
| Hv 290 | TAA1 | CD2 or TAA2 | TAA1 | CD3 | CD3 | TAA1 |
| Hv 291 | TAA1 | TAA1 | CD2 or TAA2 | CD3 | CD3 | TAA1 |
| Hv 292 | TAA1 | TAA1 | TAA1 | CD3 | CD3 | CD2 or TAA2 |
| Hv 293 | CD2 or TAA2 | TAA1 | TAA1 | CD3 | TAA1 | CD3 |
| Hv 294 | TAA1 | CD2 or TAA2 | TAA1 | CD3 | TAA1 | CD3 |
| Hv 295 | TAA1 | TAA1 | CD2 or TAA2 | CD3 | TAA1 | CD3 |
| Hv 296 | TAA1 | TAA1 | TAA1 | CD3 | CD2 or TAA2 | CD3 |
| Hv 297 | CD2 or TAA2 | TAA1 | TAA1 | TAA1 | CD3 | CD3 |
| Hv 298 | TAA1 | CD2 or TAA2 | TAA1 | TAA1 | CD3 | CD3 |
| Hv 299 | TAA1 | TAA1 | CD2 or TAA2 | TAA1 | CD3 | CD3 |
| Hv 300 | TAA1 | TAA1 | TAA1 | CD2 or TAA2 | CD3 | CD3 |
| Hv 301 | CD3 | CD2 or TAA2 | TAA1 | TAA1 | TAA1 | TAA1 |
| Hv 302 | CD3 | TAA1 | CD2 or TAA2 | TAA1 | TAA1 | TAA1 |
| Hv 303 | CD3 | TAA1 | TAA1 | CD2 or TAA2 | TAA1 | TAA1 |
| Hv 304 | CD3 | TAA1 | TAA1 | TAA1 | CD2 or TAA2 | TAA1 |
| Hv 305 | CD3 | TAA1 | TAA1 | TAA1 | TAA1 | CD2 or TAA2 |
| Hv 306 | CD2 or TAA2 | CD3 | TAA1 | TAA1 | TAA1 | TAA1 |
| Hv 307 | TAA1 | CD3 | CD2 or TAA2 | TAA1 | TAA1 | TAA1 |
| Hv 308 | TAA1 | CD3 | TAA1 | CD2 or TAA2 | TAA1 | TAA1 |
| Hv 309 | TAA1 | CD3 | TAA1 | TAA1 | CD2 or TAA2 | TAA1 |
| Hv 310 | TAA1 | CD3 | TAA1 | TAA1 | TAA1 | CD2 or TAA2 |
| Hv 311 | CD2 or TAA2 | TAA1 | CD3 | TAA1 | TAA1 | TAA1 |
| Hv 312 | TAA1 | CD2 or TAA2 | CD3 | TAA1 | TAA1 | TAA1 |
| Hv 313 | TAA1 | TAA1 | CD3 | CD2 or TAA2 | TAA1 | TAA1 |
| Hv 314 | TAA1 | TAA1 | CD3 | TAA1 | CD2 or TAA2 | TAA1 |
| Hv 315 | TAA1 | TAA1 | CD3 | TAA1 | TAA1 | CD2 or TAA2 |
| Hv 316 | CD2 or TAA2 | TAA1 | TAA1 | CD3 | TAA1 | TAA1 |
| Hv 317 | TAA1 | CD2 or TAA2 | TAA1 | CD3 | TAA1 | TAA1 |
| Hv 318 | TAA1 | TAA1 | CD2 or TAA2 | CD3 | TAA1 | TAA1 |
| Hv 319 | TAA1 | TAA1 | TAA1 | CD3 | CD2 or TAA2 | TAA1 |
| Hv 320 | TAA1 | TAA1 | TAA1 | CD3 | TAA1 | CD2 or TAA2 |
| Hv 321 | CD2 or TAA2 | TAA1 | TAA1 | TAA1 | CD3 | TAA1 |
| Hv 322 | TAA1 | CD2 or TAA2 | TAA1 | TAA1 | CD3 | TAA1 |
| Hv 323 | TAA1 | TAA1 | CD2 or TAA2 | TAA1 | CD3 | TAA1 |
| Hv 324 | TAA1 | TAA1 | TAA1 | CD2 or TAA2 | CD3 | TAA1 |
| Hv 325 | TAA1 | TAA1 | TAA1 | TAA1 | CD3 | CD2 or TAA2 |
| Hv 326 | CD2 or TAA2 | TAA1 | TAA1 | TAA1 | TAA1 | CD3 |
| Hv 327 | TAA1 | CD2 or TAA2 | TAA1 | TAA1 | TAA1 | CD3 |
| Hv 328 | TAA1 | TAA1 | CD2 or TAA2 | TAA1 | TAA1 | CD3 |
| Hv 329 | TAA1 | TAA1 | TAA1 | CD2 or TAA2 | TAA1 | CD3 |
| Hv 330 | TAA1 | TAA1 | TAA1 | TAA1 | CD2 or TAA2 | CD3 |

### 7.7. Exemplary Multispecific Binding Molecules

Exemplary MBM configurations are shown FIGS. 3A-3E. The scFv, Fab, non-immunoglobulin based ABM, and Fc each can have the characteristics described for these components in Sections 7.2 to 7.4. The components of the MBM configurations can be associated with each other by any of the means described in Sections 7.3 and 7.4 (e.g., by direct bonds, ABM linkers, disulfide bonds, Fc domains with modified with knob in hole interactions, etc.). The orientations and associations of the various components shown in FIGS. 3A-3E are merely exemplary; as will be appreciated by skilled artisans, other orientations and associations can be suitable (e.g., as described in Sections 7.3 and 7.4).

MBMs are not limited to the configurations shown in FIGS. 3A-3E. Other configurations that can be used are known to those skilled in the art. See, e.g., WO 2014/145806; WO 2017/124002; Liu et al., 2017, Front Immunol. 8:38; Brinkmann & Kontermann, 2017, mAbs 9:2, 182-212; US 2016/0355600; Klein et al., 2016, MAbs 8(6):1010-20; and US 2017/0145116.

7.7.1. Exemplary MBMs

The MBMs can be bispecific, e.g., they have two antigen-binding domains, wherein one antigen-binding domain binds CD3, and and one antigen-binding domain binds a TAA.

7.7.2. Exemplary Trivalent MBMs

The MBMs can be trivalent, e.g., they have three antigen-binding domains, wherein at least one of the three antigen binding domains binds CD3, from zero to one of the three antigen binding domains binds CD2, and at least one of the three antigen binding domains binds a TAA.

7.7.3. Exemplary Tetravalent MBMs

The MBMs can be tetravalent, e.g., they have four antigen-binding domains, wherein at least one of the four antigen binding domains binds CD3, from zero to two of the four antigen binding domains binds CD2 and at least one of the four antigen binding domains binds a TAA.

### 7.8. TCR ABMs

The MBMs can contain an ABM that specifically binds to a component of a TCR complex. The TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells.

In a preferred embodiment, MBMs contain an ABM that specifically binds to CD3, for example, the CD3 antigen binding domains found in Table 1 or Table 19.

7.8.1. CD3 ABMs

The MBMs can contain an ABM that specifically binds to CD3. The term “CD3” refers to the cluster of differentiation 3 co-receptor (or co-receptor complex, or polypeptide chain of the co-receptor complex) of the T cell receptor. CD3 proteins can also include variants. CD3 proteins can also include fragments. CD3 proteins also include post-translational modifications of the CD3 amino acid sequences. Post-translational modifications include, but are not limited to, N- and O-linked glycosylation.

In some embodiments, a MBM can comprise an ABM which is an anti-CD3 antibody or an antigen-binding domain thereof. Exemplary anti-CD3 VH, VL, and scFV sequences that can be used in MBM are provided in Table 1 and Table 19.

In some embodiments, a CD3 ABM comprises the CDR sequences of NOV292. In some embodiments, a CD3 ABM comprises the CDR sequences of NOV123. In some embodiments, a CD3 ABM comprises the CDR sequences of NOV453. In some embodiments, a CD3 ABM comprises the CDR sequences of NOV229. In some embodiments, a CD3 ABM comprises the CDR sequences of NOV110. In some embodiments, a CD3 ABM comprises the CDR sequences of NOV832. In some embodiments, a CD3 ABM comprises the CDR sequences of NOV589. In some embodiments, a CD3 ABM comprises the CDR sequences of NOV580. In some embodiments, a CD3 ABM comprises the CDR sequences of NOV567. In some embodiments, a CD3 ABM comprises the CDR sequences of NOV221.

A MBM can comprise the complete heavy and light variable sequences of any of the CD3 sequences found in Table 1 or Table 19. In some embodiments, a MBM comprises a CD3 ABM which comprises the VH and VL sequences of NOV292. In some embodiments, a MBM comprises a CD3 ABM which comprises the VH and VL sequences of NOV123. In some embodiments, a MBM comprises a CD3 ABM which comprises the VH and VL sequences of NOV453. In some embodiments, a MBM comprises a CD3 ABM which comprises the VH and VL sequences of NOV229. In some embodiments, a MBM comprises a CD3 ABM which comprises the VH and VL sequences of NOV110. In some embodiments, a MBM comprises a CD3 ABM which comprises the VH and VL sequences of NOV832. In some embodiments, a MBM comprises a CD3 ABM which comprises the VH and VL sequences of NOV589. In some embodiments, a MBM comprises a CD3 ABM which comprises the VH and VL sequences of NOV580. In some embodiments, a MBM comprises a CD3 ABM which comprises the VH and VL sequences of NOV567. In some embodiments, a MBM comprises a CD3 ABM which comprises the VH and VL sequences of NOV221.

7.8.2. TCR-α/β ABMs

The MBMs can contain an ABM that specifically binds to the TCR-α chain, the TCR-13 chain, or the TCR-αβ dimer. Exemplary anti-TCR-α/β antibodies are known in the art (see, e.g., US 2012/0034221; Borst et al., 1990, Hum Immunol. 29(3):175-88 (describing antibody BMA031)). The VH, VL, and Kabat CDR sequences of antibody BMA031 are provided in Table 12.

TABLE 12
BMA031 sequences

| Domain | Sequence | SEQ ID NO: |
|---|---|---|
| BMA031 | KASGYKFTSYVMH | SEQ ID NO: 79 |
| CDR-H1 | | |
| BMA031 | YINPYNDVTKYNEKFK | SEQ ID NO: 80 |
| CDR-H2 | | |
| BMA031 | GSYYDYDGFVY | SEQ ID NO: 81 |
| CDR-H3 | | |
| BMA031 | SATSSVSYMH | SEQ ID NO: 82 |
| CDR-L1 | | |
| BMA031 | DTSKLAS | SEQ ID NO: 83 |
| CDR-L2 | | |
| BMA031 | QQWSSNPLT | SEQ ID NO: 84 |
| CDR-L3 | | |
| BMA031 | EVQLQQSGPELVKPGASVKMSCKASGYKFTSYVMHVVVKQK | SEQ ID NO: 85 |
| VH | PGQGLEWIGYINPYNDVTKYNEKFKGKATLTSDKSSSTAYME | |
| | LSSLTSEDSAVHYCARGSYYDYDGFVYWGQGTLVTVSA | |
| BMA031 | QIVLTQSPAIMSASPGEKVTMTCSATSSVSYMHVVYQQKSGT | SEQ ID NO: 86 |
| VL | SPKRWIYDTSKLASGVPARFSGSGSGTSYSLTISSMEAEDAA | |
| | TYYCQQWSSNPLTFGAGTKLELK | |

In an embodiment, a TCR ABM can comprise the CDR sequences of antibody BMA031. In other embodiments, a TCR ABM can comprise the VH and VL sequences of antibody BMA031.

7.8.3. TCR-γ/δ ABMs

The MBMs can contain an ABM that specifically binds to the TCR-γ chain, the TCR-δ chain, or the TCR-γδ dimer. Exemplary anti-TCR-γ/δ antibodies are known in the art (see, e.g., U.S. Pat. No. 5,980,892 (describing δTCS1, produced by the hybridoma deposited with the ATCC as accession number HB 9578)).

### 7.9. CD2 ABMs

7.9.1. Immunoglobulin-Based CD2 ABMs

In some embodiments, a MBM can comprise an ABM which is an anti-CD2 antibody or an antigen-binding domain thereof. Exemplary anti-CD2 antibodies are known in the art (see, e.g., U.S. Pat. No. 6,849,258, CN102827281A, US 2003/0139579 A1, and U.S. Pat. No. 5,795,572). Table 13 provides exemplary CDR, VH, and VL sequences that can be included in anti-CD2 antibodies or antigen-binding fragments thereof, for use in MBMs.

TABLE 13
Immunoglobulin Based CD2 Binders

| | | | SEQ ID |
|---|---|---|---|
| Name | Domain | Sequence | NO: |
| CD2-1 | CDR-H1 | EYYMY (Rat Lo-CD2a = BTI-322 from FIG. 33 of U.S. Pat. | SEQ ID |
| | | No. 6,849,258) | NO: 87 |
| CD2-1 | CDR-H2 | RIDPEDGSIDYVEKFKK (Rat Lo-CD2a = BTI-322 from | SEQ ID |
| | | FIG. 33 of U.S. Pat. No. 6,849,258) | NO: 88 |
| CD2-1 | CDR-H3 | GKFNYRFAY (Rat Lo-CD2a = BTI-322 from FIG. 33 of | SEQ ID |
| | | U.S. Pat. No. 6,849,258) | NO: 89 |
| CD2-1 | CDR-L1 | RSSQSLLHSSGNTYLN (Rat Lo-CD2a = BTI-322 from | SEQ ID |
| | | FIG. 31 of U.S. Pat. No. 6,849,258) | NO: 90 |
| CD2-1 | CDR-L2 | LVSKLES (Rat Lo-CD2a = BTI-322 from FIG. 31 of U.S. Pat. | SEQ ID |
| | | No. 6,849,258) | NO: 91 |
| CD2-1 | CDR-L3 | QFTHYPYT (Rat Lo-CD2a = BTI-322 from FIG. 31 of | SEQ ID |
| | | U.S. Pat. No. 6,849,258) | NO: 92 |
| CD2-1 | VH | EVQLQQSGPELQRPGASVKLSCKASGYIFTEYYMYVVV | SEQ ID |
| | | KQRPKQGLELVGRIDPEDGSIDYVEKFKKKATLTADTSS | NO: 93 |
| | | NTAYMQLSSLTSEDTATYFCARGKFNYRFAYWGQGTL | |
| | | VTVSS (SEQ ID NO: 100 of U.S. Pat. No. 6,849,258) | |
| CD2-1 | VL | DVVLTQTPPTLLATIGQSVSISCRSSQSLLHSSGNTYLN | SEQ ID |
| | | WLLQRTGQSPQPLIYLVSKLESGVPNRFSGSGSGTDFT | NO: 94 |
| | | LKISGVEAEDLGVYYCMQFTHYPYTFGAGTKLELK (Rat | |
| | | Lo-CD2a Vk from SEQ ID NO: 92, without signal | |
| | | sequence as shown in FIG. 31 of U.S. Pat. No. 6,849,258) | |
| hu1CD2-1 | VH | QVQLVQSGAEVKKPGASVKVSCKASGYTFTEYYMYVVV | SEQ ID |
| | | RQAPGQGLELMGRIDPEDGSIDYVEKFKKKVTLTADTS | NO: 95 |
| | | SSTAYMELSSLTSDDTAVYYCARGKFNYRFAYWGQGT | |
| | | LVTVSS (SEQ ID NO: 101 of U.S. Pat. No. 6,849,258) | |
| huCD2a | VL | DVVMTQSPPSLLVTLGQPASISCRSSQSLLHSSGNTYL | SEQ ID |
| | | NWLLQRPGQSPQPLIYLVSKLESGVPDRFSGSGSGTDF | NO: 96 |
| | | TLKISGVEAEDVGVYYCMQFTHYPYTFGQGTKLEIK | |
| | | (SEQ ID NO: 96 of U.S. Pat. No. 6,849,258) | |
| hu2CD2-1 | VH | EVQLQQSGPELQRPGASVKLSCKASGYIFTEYYMYVVV | SEQ ID |
| | | KQRPKQGLELVGRIDPEDGSIDYVEKFKKKATLTADTSS | NO: 97 |
| | | NTAYMQLSSLTSEDTATYFCARGKFNYRFAYWGQGTL | |
| | | VTVSS (Vh of MEDI-507; SEQ ID NO: 105 of U.S. Pat. No. | |
| | | 6,849,258) | |
| huCD2a | VL | DVVMTQSPPSLLVTLGQPASISCRSSQSLLHSSGNTYL | SEQ ID |
| | | NWLLQRPGQSPQPLIYLVSKLESGVPDRFSGSGSGTDF | NO: 98 |
| | | TLKISGVEAEDVGVYYCMQFTHYPYTFGQGTKLEIK (SEQ ID NO: 96 of | |
| | | U.S. Pat. No. 6,849,258)(same as hu1CD2-1) | |

In some embodiments, a CD2 ABM comprises the CDR sequences of CD2-1 (SEQ ID NOS: 87-92). In some embodiments, a CD2 ABM comprises the heavy and light chain variable sequences of CD2-1 (SEQ ID NO:93-94). In some embodiments, a CD2 ABM comprises the heavy and light chain variable sequences of hu1CD2-1 (SEQ ID NO:95-96). In some embodiments, a CD2 ABM comprises the heavy and light chain variable sequences of hu2CD2-1 (SEQ ID NOS:97-98).

In other embodiments, a CD2 ABM can comprise the CDR sequences of antibody 9D1 produced by the hybridoma deposited with the Chinese Culture Collection Committee General Microbiology Center on May 16, 2012 with accession no. CGMCC 6132, and which is described in CN102827281A. In other embodiments, a CD2 ABM can comprise the CDR sequences of antibody LO-CD2b produced by the hybridoma deposited with the American Type Culture Collection on Jun. 22, 1999 with accession no. PTA-802, and which is described in US 2003/0139579 A1. In yet other embodiments, a CD2 ABM can comprise the CDR sequences of the CD2 SFv-Ig produced by expression of the construct cloned in the recombinant E. coli deposited with the ATCC on Apr. 9, 1993 with accession no. 69277, and which is described in U.S. Pat. No. 5,795,572.

In other embodiments, a CD2 ABM can comprise the VH and VL sequences of antibody 9D1. In other embodiments, a CD2 ABM can comprise the VH and VL sequences of antibody LO-CD2b. In yet other embodiments, a CD2 ABM can comprise the VH and VL sequences of the CD2 SFv-Ig produced by expression of the construct cloned in the recombinant E. coli having ATCC accession no. 69277.

7.9.2. CD58-Based CD2 ABMs

In certain aspects, the present disclosure provides a MBM comprising a CD2 ABM which is a ligand. The CD2 ABM specifically binds to human CD2, whose natural ligand is CD58, also known as LFA-3. CD58/LFA-3 proteins are glycoproteins that are expressed on the surfaces of a variety of cell types (Dustin et al., 1991, Annu. Rev. Immunol. 9:27) and play roles in mediating T-cell interactions with APCs in both antigen-dependent and antigen-independent manners (Wallner et al., 1987, J. Exp. Med. 166:923). Accordingly, in certain aspects, the CD2 ABM is a CD58 moiety. As used herein, a CD58 moiety comprises an amino acid sequence comprising at least 70% sequence identity to a CD2-binding portion of CD58, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a CD2-binding portion of CD58. The sequence of human CD58 has the Uniprot identifier P19256. It has been established that CD58 fragments containing amino acid residues 30-123 of full length CD58 (i.e., the sequence designated as CD58-4 in Table 14 below) are sufficient for binding to CD2. Wang et al., 1999, Cell 97:791-803. Accordingly, in certain aspects, a CD58 moiety comprises an amino acid sequence comprising at least 70% sequence identity to amino acids 30-123 of CD58, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence designated CD58-4.

The interactions between CD58 and CD2 have been mapped through x-ray crystallography and molecular modeling. The substitution of residues E25, K29, K30, K32, D33, K34, E37, D84 and K87 (with numbering referring to the in the mature polypeptide) reduces binding to CD2. Ikemizu et al., 1999, Proc. Natl. Acad. Sci. USA 96:4289-94. Accordingly, in preferred embodiments the CD58 moiety retains the wild type residues at E25, K29, K30, K32, D33, K34, E37, D84 and K87.

In contrast, the following substitutions (with numbering referring to the full length polypeptide) did not impact binding to CD2: F29S; V37K; V49Q; V86K; T113S; and L121G. Accordingly, a CD58 moiety can include one, two, three, four, five or all six of the foregoing substitutions.

Exemplary CD58 moieties are provided in Table 14 below:

TABLE 14
CD58 sequences

| | | | SEQ ID |
|---|---|---|---|
| Name | Description | Sequence | NO: |
| CD58-1 | Full length | MVAGSDAGRALGVLSVVCLLHCFGFISCFSQQIYGVVYGNVT | SEQ ID |
| | CD58, | FHVPSNVPLKEVLWKKQKDKVAELENSEFRAFSSFKNRVYLD | NO: 99 |
| | including | TVSGSLTIYNLTSSDEDEYEMESPNITDTMKFFLYVLESLPSP | |
| | signal | TLTCALTNGSIEVQCMIPEHYNSHRGLIMYSWDCPMEQCKRN | |
| | sequence | STSIYFKMENDLPQKIQCTLSNPLFNTTSSIILTTCIPSSGHSRH | |
| | (P19256) | RYALIPIPLAVITTCIVLYMNGILKCDRKPDRTNSN | |
| CD58-2 | Extracellular | FSQQIYGVVYGNVTFHVPSNVPLKEVLVVKKQKDKVAELENSE | SEQ ID |
| | domain of | FRAFSSFKNRVYLDTVSGSLTIYNLTSSDEDEYEMESPNITDT | NO: 100 |
| | CD58, | MKFFLYVLESLPSPTLTCALTNGSIEVQCMIPEHYNSHRGLIM | |
| | corresponding | YSVVDCPMEQCKRNSTSIYFKMENDLPQKIQCTLSNPLFNTTS | |
| | to amino acids | SIILTTCIPSSGHSRHR | |
| | 29-215 of | | |
| | CD58 (WT) | | |
| CD58-3 | Extracellular | BSQQIYGVJYGNVTFHVPSNOPLKEVLWKKQKDKVAELENSE | SEQ ID |
| | domain of | FRAFSSFKNRVYLDTUSGSLTIYNLTSSDEDEYEMESPNITDX | NO: 101 |
| | CD58, | MKFFLYVZESLPSPTLTCALTNGSIEVQCMIPEHYNSHRGLIM | |
| | corresponding | YSVVDCPMEQCKRNSTSIYFKMENDLPQKIQCTLSNPLFNTTS | |
| | to amino acids | SIILTTCIPSSGHSRHR | |
| | 29-215 of | B = F or S | |
| | CD58 (with | J = V or K | |
| | permitted | O = V or Q | |
| | substitutions) | U = V or K | |
| | | X = T or S | |
| | | Z = L or G | |
| CD58-4 | Amino acids | SQQIYGVVYGNVTFHVPSNVPLKEVLVVKKQKDKVAELENSEF | SEQ ID |
| | 30-123 (WT) | RAFSSFKNRVYLDTVSGSLTIYNLTSSDEDEYEMESPNITDTM | NO: 102 |
| | | KFFLYVLES | |
| CD58-5 | Amino acids | SQQIYGVJYGNVTFHVPSNOPLKEVLVVKKQKDKVAELENSEF | SEQ ID |
| | 30-123 (with | RAFSSFKNRVYLDTVSGSLTIYNLTSSDEDEYEMESPNITDTM | NO: 103 |
| | permitted | KFFLYVLES | |
| | substitutions) | J = V or K | |
| | | O = V or Q | |

7.9.1. CD48-Based CD2 ABMs

In certain aspects the present disclosure provides a MBM comprising a CD2 ABM which is CD48 moiety. As used herein, a CD48 moiety comprises an amino acid sequence comprising at least 70% sequence identity to a CD2-binding portion of CD48, e.g., at least 70%, 71%, 72%, 73%, 74%, 75%, 76%, 77%, 78%, 79%, 80%,81%, 82%, 83%, 84%, 85%, 86%, 87%, 88%, 89%, 90%, 91%, 92%, 93%, 94%, 95%, 96%, 97%, 98%, or 99% identity to a CD2-binding portion of CD48. The sequence of human CD48 has the Uniprot identifier P09326 (www.uniprot.org/uniprot/P09326), which includes a signal peptide (amino acids 1-26) and a GPI anchor (amino acids 221-243). In certain aspects, a CD48 moiety comprises an amino acid sequence comprising at least 70% sequence identity (e.g., at least 70%, 71%, 72%, 73%, 74%, 75%, 76%, 77%, 78%, 79%, 80%,81%, 82%, 83%, 84%, 85%, 86%, 87%, 88%, 89%, 90%, 91%, 92%, 93%, 94%, 95%, 96%, 97%, 98%, or 99% identity) to the amino acid sequence of consisting of amino acids 27-220 of Uniprot identifier P09326. Human CD48 has an Ig-like C2-type I domain (amino acids 29-127 of Uniprot identifier P09326) and a Ig-like C2 type 2 domain (amino acids 132-212 of Uniprot identifier P09326). Accordingly, in some embodiments, a CD48 moiety comprises an amino acid sequence comprising at least 70% sequence identity (e.g., at least 70%, 71%, 72%, 73%, 74%, 75%, 76%, 77%, 78%, 79%, 80%,81%, 82%, 83%, 84%, 85%, 86%, 87%, 88%, 89%, 90%, 91%, 92%, 93%, 94%, 95%, 96%, 97%, 98%, or 99% identity) to the amino acid sequence of consisting of amino acids 29-212 of Uniprot identifier P09326, to the 02-type I domain (amino acids 29-127 of Uniprot identifier P09326) and/or to the Ig-like C2 type 2 domain (amino acids 132-212 of Uniprot identifier P09326). A CD48 moiety can in some embodiments comprise one or more natural variants relative to the sequence of Uniprot identifier P09326. For example, a CD48 moiety can include a E102Q substitution. As another example, a CD48 moiety can comprise an amino acid sequence corresponding to a CD-48 isoform or a CD2 binding portion thereof, e.g., the isoform having Uniprot identifier P09326-2 or a CD2 binding portion thereof.

### 7.10. Tumor-Associated Antigen ABMs

The MBMs can comprise at least one ABM that binds specifically to a tumor-associated antigen (TAA). For example, a BBM can comprise an ABM2 that specifically binds a TAA and a TBM can comprise an ABM2 that specifically binds a TAA (“TAA 1”) and an AMB3 that specifically binds different TAA (“TAA 2”). Preferably, the TAA (or each TAA, in the case of TAA 1 and TAA 2) is a human TAA. The antigen may or may not be present on normal cells. In certain embodiments, the TAA is preferentially expressed or upregulated on tumor cells as compared to normal cells. In other embodiments, the TAA is a lineage marker.

It is anticipated that any type of tumor and any type of TAA can be targeted by the MBMs. Exemplary types of cancers that can be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs are known for virtually any type of cancer.

Exemplary types of B cell malignancies that may be targeted include Hodgkin's lymphomas, non-Hodgkin's lymphomas (NHLs), and multiple myeloma. Examples of NHLs include diffuse large B-cell lymphoma (DLBCL), follicular lymphoma, chronic lymphocytic leukemia (CLL)/small lymphocytic lymphoma (SLL), mantle cell lymphoma (MCL), marginal zone lymphomas, Burkitt lymphoma, lymphoplasmacytic lymphoma (Waldenstrom macroglobulinemia), hairy cell leukemia, primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, and primary effusion lymphoma.

Exemplary TAAs for which a MBM can be created (e.g., targeted by ABM2 and/or ABM3) include ABCF1; ACVR1; ACVR1B; ACVR2; ACVR2B; ACVRL1; ADORA2A; ADRB3; Aggrecan; AGR2; AICDA; AIF1; AIG1; AKAP1; AKAP2; ALK; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; Cadherin 17; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD32b; CD40; CD40L; CD44; CD45RB; CD52; CD69; CD72; CD74; CD79A; CD79B; CD8; CD80; CD81; CD83; CD86; CD97; CD179a; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN6; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (1-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; C1q; C1r; C1; C4a; C4b; C2a; C2b; C3a; C3b; DAB21P; DES; DKFZp451J0118; DNCL1; DPP4; E-selectin; E2F1; ECGF1; EDG1; EFNA1; EFNA3; EFNB2; EGF; EGFR; EGFRvIII; ELAC2; ENG; ENO1; ENO2; ENO3; EPHB4; EPO; ERBB2 (Her-2); EREG; ERK8; ESR1; ESR2; F3 (TF); Factor VII; Factor IX; Factor V; Factor VIIa; Factor Factor X; Factor XII; Factor XIII; FADD; FasL; FASN; FCER1A; FCER2; Fc gamma receptor; FCGR3A; FCRL5; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; Folate receptor alpha; Folate receptor beta; FOS; FOSL1 (FRA-1); Fucosyl GM1; FY (DARC); GABRP (GABAa); GAGEB1; GAGEC1; GALNAC4S-6ST; GATA3; GDF5; GFI1; GGT1; GM-CSF; GloboH; GNAS1; GNRH1; GPNMB; GPR2 (CCR10); GPR20; GPR31; GPR44; GPR64; GPR81 (FKSG80); GPRC5D; GRCC10 (010); GRP; GSN (Gelsolin); GSTP1; glycoprotein (gP)IIb/IIIa; HAVCR1; HAVCR2; HDAC4; HDAC5; HDAC7A; HDAC9; Her2; HER3; HGF; HIF1A; HIP1; histamine and histamine receptors; HLA-A; HLA-DRA; HM74; HMGB1; HMOX1; HMWMAA; HUMCYT2A; ICEBERG; ICOSL; ID2; IFN-a; IFNA1; IFNA2; IFNA4; IFNA5; IFNA6; IFNA7; IFNB1; IFN-γ; IFNW1; IGBP1; IGF1; IGF1R; IGF2; IGFBP2; IGFBP3; IGFBP6; IL-1; IL-α; IL-1-β; IL10; IL10 RA; IL10RB; IL11; IL11 RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMAS; LEP (leptin); Lingo-p75; Lingo-Troy; LRP6; LPS; LTA (TNF-b); LTB; LTB4R (GPR16); LTB4R2; LTBR; LY6K; LYPD8; MACMARCKS; MAG or Omgp; MAP2K7 (c-Jun); MDK; mesothelin; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; NY-BR-1; o-acetyl-GD2; ODZ1; OPRD1; OR51E2; P2RX7; PANX3; PAP; PART1; PATE; PAWR; PCA3; PCNA; PDGFA; PDGFB; PECAM1; PF4 (CXCL4); PGE2; PGF; PGR; phosphacan; PIAS2; PIK3CG; PLAC1; plasminogen activator; PLAU (uPA); PLG; PLXDC1; polysialic acid; PPBP (CXCL7); PPID; PR1; PRKCQ; PRKD1; PRL; PROC; Protein C; PROK2; PSAP; PSCA; PTAFR; PTEN; PTGS2 (COX-2); PTN; RAC2 (p21Rac2); RAGE; RARB; RGS1; RGS13; RGS3; RNF110 (ZNF144); ROBO2; SIO0A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC34A2; SLC39A6; SLC43A1; SLIT2; SLITRK6; SPP1; SPRR1B (Spr1); ST6GAL1; STAB1; STATE; STEAP; STEAP2; substance P; TACSTD2; TB4R2; TBX21; TCP10; TDGF1; TEK; TEM1/CD248; TEM7R; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-α; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase ha); TP53; TPM1; TPM2; TRADD; TRAF1; TRAF2; TRAF3; TRAF4; TRAF5; TRAF6; TREM1; TREM2; TRPC6; TSHR; TSLP; TWEAK; thrombomodulin; thrombin; UPK2; VEGF; VEGFB; VEGFC; versican; VHL C5; VLA-4; XCL1 (lymphotactin); XCL2 (SCM-1b); XCR1 (GPRS/CCXCR1); YY1; and ZFPM2.

In some embodiments, a TAA targeted by a MBM is ADRB3. In some embodiments, a TAA targeted by a MBM is AKAP-4. In some embodiments, a TAA targeted by a MBM is ALK. In some embodiments, a TAA targeted by a MBM is androgen receptor. In some embodiments, a TAA targeted by a MBM is B7H3. In some embodiments, a TAA targeted by a MBM is BCMA. In some embodiments, a TAA targeted by a MBM is BORIS. In some embodiments, a TAA targeted by a MBM is BST2. In some embodiments, a TAA targeted by a MBM is Cadherin17. In some embodiments, a TAA targeted by a MBM is CAIX. In some embodiments, a TAA targeted by a MBM is CD171. In some embodiments, a TAA targeted by a MBM is CD179a. In some embodiments, a TAA targeted by a MBM is CD19. In some embodiments, a TAA targeted by a MBM is CD20. In some embodiments, a TAA targeted by a MBM is CD22. In some embodiments, a TAA targeted by a MBM is CD24. In some embodiments, a TAA targeted by a MBM is CD30. In some embodiments, a TAA targeted by a MBM is CD300LF. In some embodiments, a TAA targeted by a MBM is CD32b. In some embodiments, a TAA targeted by a MBM is CD33. In some embodiments, a TAA targeted by a MBM is CD38. In some embodiments, a TAA targeted by a MBM is CD44v6. In some embodiments, a TAA targeted by a MBM is CD72. In some embodiments, a TAA targeted by a MBM is CD79a. In some embodiments, a TAA targeted by a MBM is CD79b. In some embodiments, a TAA targeted by a MBM is CD97. In some embodiments, a TAA targeted by a MBM is CEA. In some embodiments, a TAA targeted by a MBM is CLDN6. In some embodiments, a TAA targeted by a MBM is CLEC12A. In some embodiments, a TAA targeted by a MBM is CLL-1. In some embodiments, a TAA targeted by a MBM is CS-1. In some embodiments, a TAA targeted by a MBM is CXORF61. In some embodiments, a TAA targeted by a MBM is Cyclin B1. In some embodiments, a TAA targeted by a MBM is CYP1B1. In some embodiments, a TAA targeted by a MBM is EGFR. In some embodiments, a TAA targeted by a MBM is EGFRvIII. In some embodiments, a TAA targeted by a MBM is EMR2. In some embodiments, a TAA targeted by a MBM is EPCAM. In some embodiments, a TAA targeted by a MBM is EphA2. In some embodiments, a TAA targeted by a MBM is EphB2. In some embodiments, a TAA targeted by a MBM is ERBB2. In some embodiments, a TAA targeted by a MBM is ERG (TMPRSS2 ETS fusion gene). In some embodiments, a TAA targeted by a MBM is ETV6-AML. In some embodiments, a TAA targeted by a MBM is FAP. In some embodiments, a TAA targeted by a MBM is FCAR. In some embodiments, a TAA targeted by a MBM is FCRL5. In some embodiments, a TAA targeted by a MBM is FLT3. In some embodiments, a TAA targeted by a MBM is FLT3. In some embodiments, a TAA targeted by a MBM is folate receptor alpha. In some embodiments, a TAA targeted by a MBM is folate receptor beta. In some embodiments, a TAA targeted by a MBM is Fos-related antigen 1. In some embodiments, a TAA targeted by a MBM is fucosyl GM1. In some embodiments, a TAA targeted by a MBM is GD2. In some embodiments, a TAA targeted by a MBM is GD2. In some embodiments, a TAA targeted by a MBM is GD3. In some embodiments, a TAA targeted by a MBM is GloboH. In some embodiments, a TAA targeted by a MBM is GM3. In some embodiments, a TAA targeted by a MBM is gp100Tn. In some embodiments, a TAA targeted by a MBM is GPC3. In some embodiments, a TAA targeted by a MBM is GPNMB. In some embodiments, a TAA targeted by a MBM is GPR20. In some embodiments, a TAA targeted by a MBM is GPRC5D. In some embodiments, a TAA targeted by a MBM is GPR64. In some embodiments, a TAA targeted by a MBM is HAVCR1. In some embodiments, a TAA targeted by a MBM is HER3. In some embodiments, a TAA targeted by a MBM is HMWMAA. In some embodiments, a TAA targeted by a MBM is hTERT. In some embodiments, a TAA targeted by a MBM is Igf-I receptor. In some embodiments, a TAA targeted by a MBM is IGLL1. In some embodiments, a TAA targeted by a MBM is IL-11Ra. In some embodiments, a TAA targeted by a MBM is IL-13Ra2. In some embodiments, a TAA targeted by a MBM is KIT. In some embodiments, a TAA targeted by a MBM is LAIR1. In some embodiments, a TAA targeted by a MBM is LCK. In some embodiments, a TAA targeted by a MBM is LewisY. In some embodiments, a TAA targeted by a MBM is LILRA2. In some embodiments, a TAA targeted by a MBM is LMP2. In some embodiments, a TAA targeted by a MBM is LRP6. In some embodiments, a TAA targeted by a MBM is LY6K. In some embodiments, a TAA targeted by a MBM is LY75. In some embodiments, a TAA targeted by a MBM is LYPD8. In some embodiments, a TAA targeted by a MBM is MAD-CT-1. In some embodiments, a TAA targeted by a MBM is MAD-CT-2. In some embodiments, a TAA targeted by a MBM is mesothelin. In some embodiments, a TAA targeted by a MBM is ML-IAP. In some embodiments, a TAA targeted by a MBM is MUC1. In some embodiments, a TAA targeted by a MBM is MYCN. In some embodiments, a TAA targeted by a MBM is NA17. In some embodiments, a TAA targeted by a MBM is NCAM. In some embodiments, a TAA targeted by a MBM is NKG2D. In some embodiments, a TAA targeted by a MBM is NY-BR-1. In some embodiments, a TAA targeted by a MBM is o-acetyl-GD2. In some embodiments, a TAA targeted by a MBM is OR51E2. In some embodiments, a TAA targeted by a MBM is OY-TES1. In some embodiments, a TAA targeted by a MBM is a p53 mutant. In some embodiments, a TAA targeted by a MBM is PANX3. In some embodiments, a TAA targeted by a MBM is PAX3. In some embodiments, a TAA targeted by a MBM is PAX5. In some embodiments, a TAA targeted by a MBM is PDGFR-beta. In some embodiments, a TAA targeted by a MBM is PLAC1. In some embodiments, a TAA targeted by a MBM is polysialic acid. In some embodiments, a TAA targeted by a MBM is PRSS21. In some embodiments, a TAA targeted by a MBM is PSCA. In some embodiments, a TAA targeted by a MBM is RhoC. In some embodiments, a TAA targeted by a MBM is ROR1. In some embodiments, a TAA targeted by a MBM is a sarcoma translocation breakpoint protein. In some embodiments, a TAA targeted by a MBM is SART3. In some embodiments, a TAA targeted by a MBM is SLC34A2. In some embodiments, a TAA targeted by a MBM is SLC39A6. In some embodiments, a TAA targeted by a MBM is sLe. In some embodiments, a TAA targeted by a MBM is SLITRK6. In some embodiments, a TAA targeted by a MBM is sperm protein 17. In some embodiments, a TAA targeted by a MBM is SSEA-4. In some embodiments, a TAA targeted by a MBM is SSX2. In some embodiments, a TAA targeted by a MBM is TAAG72. In some embodiments, a TAA targeted by a MBM is TAARP. In some embodiments, a TAA targeted by a MBM is TACSTD2. In some embodiments, a TAA targeted by a MBM is TEM1/CD248. In some embodiments, a TAA targeted by a MBM is TEM7R. In some embodiments, a TAA targeted by a MBM is TGS5. In some embodiments, a TAA targeted by a MBM is Tie 2. In some embodiments, a TAA targeted by a MBM is Tn Ag. In some embodiments, a TAA targeted by a MBM is TSHR. In some embodiments, a TAA targeted by a MBM is tyrosinase. In some embodiments, a TAA targeted by a MBM is UPK2. In some embodiments, a TAA targeted by a MBM is VEGFR2. In some embodiments, a TAA targeted by a MBM is WT1. In some embodiments, a TAA targeted by a MBM is XAGE1.

In some embodiments, a TAA targeted by a MBM is selected from BCMA, CD19, CD20, CD22, CD123, CD33, CLL1, CD138 (also known as Syndecan-1, SDC1), CS1, CD38, CD133, FLT3, CD52, TNFRSF13C (TNF Receptor Superfamily Member 13C, also known as BAFFR: B-Cell-Activating Factor Receptor), TNFRSF13B (TNF Receptor Superfamily Member 13B, also known as TACI: Transmembrane Activator And CAML Interactor), CXCR4 (C-X-C Motif Chemokine Receptor 4), PD-L1 (programmed death-ligand 1), LY9 (lymphocyte antigen 9, also known as CD229), CD200, FCGR2B (Fc fragment of IgG receptor IIb, also known as CD32b), CD21, CD23, CD24, CD40L, CD72, CD79a, and CD79b.

In some embodiments a TAA targeted by a MBM is CD19. In some embodiments, a TAA targeted by a MBM is BCMA. In some embodiments, a TAA targeted by a MBM is CD20. In some embodiments, a TAA targeted by a MBM is CD22. In some embodiments, a TAA targeted by a MBM is CD123. In some embodiments, a TAA targeted by a MBM is CD33. In some embodiments, a TAA targeted by a MBM is CLL1. In some embodiments, a TAA targeted by a MBM is CD138. In some embodiments, a TAA targeted by a MBM is CS1. In some embodiments, a TAA targeted by a MBM is CD38. In some embodiments, a TAA targeted by a MBM is CD133. In some embodiments, a TAA targeted by a MBM is FLT3. In some embodiments, a TAA targeted by a MBM is CD52. In some embodiments, a TAA targeted by a MBM is TNFRSF13C. In some embodiments, a TAA targeted by a MBM is TNFRSF13B. In some embodiments, a TAA targeted by a MBM is CXCR4. In some embodiments, a TAA targeted by a MBM is PD-L1. In some embodiments, a TAA targeted by a MBM is LY9. In some embodiments, a TAA targeted by a MBM is CD200. In some embodiments, a TAA targeted by a MBM is CD21. In some embodiments, a TAA targeted by a MBM is CD23. In some embodiments, a TAA targeted by a MBM is CD24. In some embodiments, a TAA targeted by a MBM is CD40L. In some embodiments, a TAA targeted by a MBM is CD72. In some embodiments, a TAA targeted by a MBM is CD79a. In some embodiments, a TAA targeted by a MBM is CD79b.

In some embodiments, a MBM targets two TAAs (TAA 1 and TAA 2) selected from the TAAs described in this Section.

In some embodiments, TAA 1 is CD19 and TAA 2 is CD20 (or vice versa). In some embodiments, TAA 1 is CD19 and TAA 2 is CD22 (or vice versa). In some embodiments, TAA 1 is CD19 and TAA 2 is CD123 (or vice versa). In some embodiments, TAA 1 is CD19 and TAA 2 is BCMA (or vice versa). In some embodiments, TAA 1 is CD19 and TAA 2 is CD33 (or vice versa). In some embodiments, TAA 1 is CD19 and TAA 2 is CLL1 (or vice versa). In some embodiments, TAA 1 is CD19 and TAA 2 is CD138 (or vice versa). In some embodiments, TAA 1 is CD19 and TAA 2 is CS1 (or vice versa). In some embodiments, TAA 1 is CD19 and TAA 2 is CD38 (or vice versa). In some embodiments, TAA 1 is CD19 and TAA 2 is CD133 (or vice versa). In some embodiments, TAA 1 is CD19 and TAA 2 is FLT3 (or vice versa). In some embodiments, TAA 1 is CD19 and TAA 2 is CD52 (or vice versa). In some embodiments, TAA 1 is CD19 and TAA 2 is TNFRSF13C (or vice versa). In some embodiments, TAA 1 is CD19 and TAA 2 is TNFRSF13B (or vice versa). In some embodiments, TAA 1 is CD19 and TAA 2 is CXCR4 (or vice versa). In some embodiments, TAA 1 is CD19 and TAA 2 is PD-L1 (or vice versa). In some embodiments, TAA 1 is CD19 and TAA 2 is LY9 (or vice versa). In some embodiments, TAA 1 is CD19 and TAA 2 is CD200 (or vice versa). In some embodiments, TAA 1 is CD19 and TAA 2 is FCGR2B (or vice versa). In some embodiments, TAA 1 is CD19 and TAA 2 is CD21 (or vice versa). In some embodiments, TAA 1 is CD19 and TAA 2 is CD23 (or vice versa). In some embodiments, TAA 1 is CD19 and TAA 2 is CD24 (or vice versa). In some embodiments, TAA 1 is CD19 and TAA 2 is CD40L (or vice versa). In some embodiments, TAA 1 is CD19 and TAA 2 is CD72 (or vice versa). In some embodiments, TAA 1 is CD19 and TAA 2 is CD79a (or vice versa). In some embodiments, TAA 1 is CD19 and TAA 2 is CD79b (or vice versa). In some embodiments, TAA 1 is CD20 and TAA 2 is CD22 (or vice versa). In some embodiments, TAA 1 is CD20 and TAA 2 is CD123 (or vice versa). In some embodiments, TAA 1 is CD20 and TAA 2 is BCMA (or vice versa). In some embodiments, TAA 1 is CD20 and TAA 2 is CD33 (or vice versa). In some embodiments, TAA 1 is CD20 and TAA 2 is CLL1 (or vice versa). In some embodiments, TAA 1 is CD20 and TAA 2 is CD138 (or vice versa). In some embodiments, TAA 1 is CD20 and TAA 2 is CS1 (or vice versa). In some embodiments, TAA 1 is CD20 and TAA 2 is CD38 (or vice versa). In some embodiments, TAA 1 is CD20 and TAA 2 is CD133 (or vice versa). In some embodiments, TAA 1 is CD20 and TAA 2 is FLT3 (or vice versa). In some embodiments, TAA 1 is CD20 and TAA 2 is CD52 (or vice versa). In some embodiments, TAA 1 is CD20 and TAA 2 is TNFRSF13C (or vice versa). In some embodiments, TAA 1 is CD20 and TAA 2 is TNFRSF13B (or vice versa). In some embodiments, TAA 1 is CD20 and TAA 2 is CXCR4 (or vice versa). In some embodiments, TAA 1 is CD20 and TAA 2 is PD-L1 (or vice versa). In some embodiments, TAA 1 is CD20 and TAA 2 is LY9 (or vice versa). In some embodiments, TAA 1 is CD20 and TAA 2 is CD200 (or vice versa). In some embodiments, TAA 1 is CD20 and TAA 2 is FCGR2B (or vice versa). In some embodiments, TAA 1 is CD20 and TAA 2 is CD21 (or vice versa). In some embodiments, TAA 1 is CD20 and TAA 2 is CD23 (or vice versa). In some embodiments, TAA 1 is CD20 and TAA 2 is CD24 (or vice versa). In some embodiments, TAA 1 is CD20 and TAA 2 is CD40L (or vice versa). In some embodiments, TAA 1 is CD20 and TAA 2 is CD72 (or vice versa). In some embodiments, TAA 1 is CD20 and TAA 2 is CD79a (or vice versa). In some embodiments, TAA 1 is CD20 and TAA 2 is CD79b (or vice versa). In some embodiments, TAA 1 is CD22 and TAA 2 is CD123 (or vice versa). In some embodiments, TAA 1 is CD22 and TAA 2 is BCMA (or vice versa). In some embodiments, TAA 1 is CD22 and TAA 2 is CD33 (or vice versa). In some embodiments, TAA 1 is CD22 and TAA 2 is CLL1 (or vice versa). In some embodiments, TAA 1 is CD22 and TAA 2 is CD138 (or vice versa). In some embodiments, TAA 1 is CD22 and TAA 2 is CS1 (or vice versa). In some embodiments, TAA 1 is CD22 and TAA 2 is CD38 (or vice versa). In some embodiments, TAA 1 is CD22 and TAA 2 is CD133 (or vice versa). In some embodiments, TAA 1 is CD22 and TAA 2 is FLT3 (or vice versa). In some embodiments, TAA 1 is CD22 and TAA 2 is CD52 (or vice versa). In some embodiments, TAA 1 is CD22 and TAA 2 is TNFRSF13C (or vice versa). In some embodiments, TAA 1 is CD22 and TAA 2 is TNFRSF13B (or vice versa). In some embodiments, TAA 1 is CD22 and TAA 2 is CXCR4 (or vice versa). In some embodiments, TAA 1 is CD22 and TAA 2 is PD-L1 (or vice versa). In some embodiments, TAA 1 is CD22 and TAA 2 is LY9 (or vice versa). In some embodiments, TAA 1 is CD22 and TAA 2 is CD200 (or vice versa). In some embodiments, TAA 1 is CD22 and TAA 2 is FCGR2B (or vice versa). In some embodiments, TAA 1 is CD22 and TAA 2 is CD21 (or vice versa). In some embodiments, TAA 1 is CD22 and TAA 2 is CD23 (or vice versa). In some embodiments, TAA 1 is CD22 and TAA 2 is CD24 (or vice versa). In some embodiments, TAA 1 is CD22 and TAA 2 is CD40L (or vice versa). In some embodiments, TAA 1 is CD22 and TAA 2 is CD72 (or vice versa). In some embodiments, TAA 1 is CD22 and TAA 2 is CD79a (or vice versa). In some embodiments, TAA 1 is CD22 and TAA 2 is CD79b (or vice versa). In some embodiments, TAA 1 is CD123 and TAA 2 is BCMA (or vice versa). In some embodiments, TAA 1 is CD123 and TAA 2 is CD33 (or vice versa). In some embodiments, TAA 1 is CD123 and TAA 2 is CLL1 (or vice versa). In some embodiments, TAA 1 is CD123 and TAA 2 is CD138 (or vice versa). In some embodiments, TAA 1 is CD123 and TAA 2 is CS1 (or vice versa). In some embodiments, TAA 1 is CD123 and TAA 2 is CD38 (or vice versa). In some embodiments, TAA 1 is CD123 and TAA 2 is CD133 (or vice versa). In some embodiments, TAA 1 is CD123 and TAA 2 is FLT3 (or vice versa). In some embodiments, TAA 1 is CD123 and TAA 2 is CD52 (or vice versa). In some embodiments, TAA 1 is CD123 and TAA 2 is TNFRSF13C (or vice versa). In some embodiments, TAA 1 is CD123 and TAA 2 is TNFRSF13B (or vice versa). In some embodiments, TAA 1 is CD123 and TAA 2 is CXCR4 (or vice versa). In some embodiments, TAA 1 is CD123 and TAA 2 is PD-L1 (or vice versa). In some embodiments, TAA 1 is CD123 and TAA 2 is LY9 (or vice versa). In some embodiments, TAA 1 is CD123 and TAA 2 is CD200 (or vice versa). In some embodiments, TAA 1 is CD123 and TAA 2 is FCGR2B (or vice versa). In some embodiments, TAA 1 is CD123 and TAA 2 is CD21 (or vice versa). In some embodiments, TAA 1 is CD123 and TAA 2 is CD23 (or vice versa). In some embodiments, TAA 1 is CD123 and TAA 2 is CD24 (or vice versa). In some embodiments, TAA 1 is CD123 and TAA 2 is CD40L (or vice versa). In some embodiments, TAA 1 is CD123 and TAA 2 is CD72 (or vice versa). In some embodiments, TAA 1 is CD123 and TAA 2 is CD79a (or vice versa). In some embodiments, TAA 1 is CD123 and TAA 2 is CD79b (or vice versa). In some embodiments, TAA 1 is BCMA and TAA 2 is CD33 (or vice versa). In some embodiments, TAA 1 is BCMA and TAA 2 is CLL1 (or vice versa). In some embodiments, TAA 1 is BCMA and TAA 2 is CD138 (or vice versa). In some embodiments, TAA 1 is BCMA and TAA 2 is CS1 (or vice versa). In some embodiments, TAA 1 is BCMA and TAA 2 is CD38 (or vice versa). In some embodiments, TAA 1 is BCMA and TAA 2 is CD133 (or vice versa). In some embodiments, TAA 1 is BCMA and TAA 2 is FLT3 (or vice versa). In some embodiments, TAA 1 is BCMA and TAA 2 is CD52 (or vice versa). In some embodiments, TAA 1 is BCMA and TAA 2 is TNFRSF13C (or vice versa). In some embodiments, TAA 1 is BCMA and TAA 2 is TNFRSF13B (or vice versa). In some embodiments, TAA 1 is BCMA and TAA 2 is CXCR4 (or vice versa). In some embodiments, TAA 1 is BCMA and TAA 2 is PD-L1 (or vice versa). In some embodiments, TAA 1 is BCMA and TAA 2 is LY9 (or vice versa). In some embodiments, TAA 1 is BCMA and TAA 2 is CD200 (or vice versa). In some embodiments, TAA 1 is BCMA and TAA 2 is FCGR2B (or vice versa). In some embodiments, TAA 1 is BCMA and TAA 2 is CD21 (or vice versa). In some embodiments, TAA 1 is BCMA and TAA 2 is CD23 (or vice versa). In some embodiments, TAA 1 is BCMA and TAA 2 is CD24 (or vice versa). In some embodiments, TAA 1 is BCMA and TAA 2 is CD40L (or vice versa). In some embodiments, TAA 1 is BCMA and TAA 2 is CD72 (or vice versa). In some embodiments, TAA 1 is BCMA and TAA 2 is CD79a (or vice versa). In some embodiments, TAA 1 is BCMA and TAA 2 is CD79b (or vice versa). In some embodiments, TAA 1 is CD33 and TAA 2 is CLL1 (or vice versa). In some embodiments, TAA 1 is CD33 and TAA 2 is CD138 (or vice versa). In some embodiments, TAA 1 is CD33 and TAA 2 is CS1 (or vice versa). In some embodiments, TAA 1 is CD33 and TAA 2 is CD38 (or vice versa). In some embodiments, TAA 1 is CD33 and TAA 2 is CD133 (or vice versa). In some embodiments, TAA 1 is CD33 and TAA 2 is FLT3 (or vice versa). In some embodiments, TAA 1 is CD33 and TAA 2 is CD52 (or vice versa). In some embodiments, TAA 1 is CD33 and TAA 2 is TNFRSF13C (or vice versa). In some embodiments, TAA 1 is CD33 and TAA 2 is TNFRSF13B (or vice versa). In some embodiments, TAA 1 is CD33 and TAA 2 is CXCR4 (or vice versa). In some embodiments, TAA 1 is CD33 and TAA 2 is PD-L1 (or vice versa). In some embodiments, TAA 1 is CD33 and TAA 2 is LY9 (or vice versa). In some embodiments, TAA 1 is CD33 and TAA 2 is CD200 (or vice versa). In some embodiments, TAA 1 is CD33 and TAA 2 is FCGR2B (or vice versa). In some embodiments, TAA 1 is CD33 and TAA 2 is CD21 (or vice versa). In some embodiments, TAA 1 is CD33 and TAA 2 is CD23 (or vice versa). In some embodiments, TAA 1 is CD33 and TAA 2 is CD24 (or vice versa). In some embodiments, TAA 1 is CD33 and TAA 2 is CD40L (or vice versa). In some embodiments, TAA 1 is CD33 and TAA 2 is CD72 (or vice versa). In some embodiments, TAA 1 is CD33 and TAA 2 is CD79a (or vice versa). In some embodiments, TAA 1 is CD33 and TAA 2 is CD79b (or vice versa). In some embodiments, TAA 1 is CLL1 and TAA 2 is CD138 (or vice versa). In some embodiments, TAA 1 is CLL1 and TAA 2 is CS1 (or vice versa). In some embodiments, TAA 1 is CLL1 and TAA 2 is CD38 (or vice versa). In some embodiments, TAA 1 is CLL1 and TAA 2 is CD133 (or vice versa). In some embodiments, TAA 1 is CLL1 and TAA 2 is FLT3 (or vice versa). In some embodiments, TAA 1 is CLL1 and TAA 2 is CD52 (or vice versa). In some embodiments, TAA 1 is CLL1 and TAA 2 is TNFRSF13C (or vice versa). In some embodiments, TAA 1 is CLL1 and TAA 2 is TNFRSF13B (or vice versa). In some embodiments, TAA 1 is CLL1 and TAA 2 is CXCR4 (or vice versa). In some embodiments, TAA 1 is CLL1 and TAA 2 is PD-L1 (or vice versa). In some embodiments, TAA 1 is CLL1 and TAA 2 is LY9 (or vice versa). In some embodiments, TAA 1 is CLL1 and TAA 2 is CD200 (or vice versa). In some embodiments, TAA 1 is CLL1 and TAA 2 is FCGR2B (or vice versa). In some embodiments, TAA 1 is CLL1 and TAA 2 is CD21 (or vice versa). In some embodiments, TAA 1 is CLL1 and TAA 2 is CD23 (or vice versa). In some embodiments, TAA 1 is CLL1 and TAA 2 is CD24 (or vice versa). In some embodiments, TAA 1 is CLL1 and TAA 2 is CD40L (or vice versa). In some embodiments, TAA 1 is CLL1 and TAA 2 is CD72 (or vice versa). In some embodiments, TAA 1 is CLL1 and TAA 2 is CD79a (or vice versa). In some embodiments, TAA 1 is CLL1 and TAA 2 is CD79b (or vice versa). In some embodiments, TAA 1 is CD138 and TAA 2 is CS1 (or vice versa). In some embodiments, TAA 1 is CD138 and TAA 2 is CD38 (or vice versa). In some embodiments, TAA 1 is CD138 and TAA 2 is CD133 (or vice versa). In some embodiments, TAA 1 is CD138 and TAA 2 is FLT3 (or vice versa). In some embodiments, TAA 1 is CD138 and TAA 2 is CD52 (or vice versa). In some embodiments, TAA 1 is CD138 and TAA 2 is TNFRSF13C (or vice versa). In some embodiments, TAA 1 is CD138 and TAA 2 is TNFRSF13B (or vice versa). In some embodiments, TAA 1 is CD138 and TAA 2 is CXCR4 (or vice versa). In some embodiments, TAA 1 is CD138 and TAA 2 is PD-L1 (or vice versa). In some embodiments, TAA 1 is CD138 and TAA 2 is LY9 (or vice versa). In some embodiments, TAA 1 is CD138 and TAA 2 is CD200 (or vice versa). In some embodiments, TAA 1 is CD138 and TAA 2 is FCGR2B (or vice versa). In some embodiments, TAA 1 is CD138 and TAA 2 is CD21 (or vice versa). In some embodiments, TAA 1 is CD138 and TAA 2 is CD23 (or vice versa). In some embodiments, TAA 1 is CD138 and TAA 2 is CD24 (or vice versa). In some embodiments, TAA 1 is CD138 and TAA 2 is CD40L (or vice versa). In some embodiments, TAA 1 is CD138 and TAA 2 is CD72 (or vice versa). In some embodiments, TAA 1 is CD138 and TAA 2 is CD79a (or vice versa). In some embodiments, TAA 1 is CD138 and TAA 2 is CD79b (or vice versa). In some embodiments, TAA 1 is CS1 and TAA 2 is CD38 (or vice versa). In some embodiments, TAA 1 is CS1 and TAA 2 is CD133 (or vice versa). In some embodiments, TAA 1 is CS1 and TAA 2 is FLT3 (or vice versa). In some embodiments, TAA 1 is CS1 and TAA 2 is CD52 (or vice versa). In some embodiments, TAA 1 is CS1 and TAA 2 is TNFRSF13C (or vice versa). In some embodiments, TAA 1 is CS1 and TAA 2 is TNFRSF13B (or vice versa). In some embodiments, TAA 1 is CS1 and TAA 2 is CXCR4 (or vice versa). In some embodiments, TAA 1 is CS1 and TAA 2 is PD-L1 (or vice versa). In some embodiments, TAA 1 is CS1 and TAA 2 is LY9 (or vice versa). In some embodiments, TAA 1 is CS1 and TAA 2 is CD200 (or vice versa). In some embodiments, TAA 1 is CS1 and TAA 2 is FCGR2B (or vice versa). In some embodiments, TAA 1 is CS1 and TAA 2 is CD21 (or vice versa). In some embodiments, TAA 1 is CS1 and TAA 2 is CD23 (or vice versa). In some embodiments, TAA 1 is CS1 and TAA 2 is CD24 (or vice versa). In some embodiments, TAA 1 is CS1 and TAA 2 is CD40L (or vice versa). In some embodiments, TAA 1 is CS1 and TAA 2 is CD72 (or vice versa). In some embodiments, TAA 1 is CS1 and TAA 2 is CD79a (or vice versa). In some embodiments, TAA 1 is CS1 and TAA 2 is CD79b (or vice versa). In some embodiments, TAA 1 is CD38 and TAA 2 is CD133 (or vice versa). In some embodiments, TAA 1 is CD38 and TAA 2 is FLT3 (or vice versa). In some embodiments, TAA 1 is CD38 and TAA 2 is CD52 (or vice versa). In some embodiments, TAA 1 is CD38 and TAA 2 is TNFRSF13C (or vice versa). In some embodiments, TAA 1 is CD38 and TAA 2 is TNFRSF13B (or vice versa). In some embodiments, TAA 1 is CD38 and TAA 2 is CXCR4 (or vice versa). In some embodiments, TAA 1 is CD38 and TAA 2 is PD-L1 (or vice versa). In some embodiments, TAA 1 is CD38 and TAA 2 is LY9 (or vice versa). In some embodiments, TAA 1 is CD38 and TAA 2 is CD200 (or vice versa). In some embodiments, TAA 1 is CD38 and TAA 2 is FCGR2B (or vice versa). In some embodiments, TAA 1 is CD38 and TAA 2 is CD21 (or vice versa). In some embodiments, TAA 1 is CD38 and TAA 2 is CD23 (or vice versa). In some embodiments, TAA 1 is CD38 and TAA 2 is CD24 (or vice versa). In some embodiments, TAA 1 is CD38 and TAA 2 is CD40L (or vice versa). In some embodiments, TAA 1 is CD38 and TAA 2 is CD72 (or vice versa). In some embodiments, TAA 1 is CD38 and TAA 2 is CD79a (or vice versa). In some embodiments, TAA 1 is CD38 and TAA 2 is CD79b (or vice versa). In some embodiments, TAA 1 is CD133 and TAA 2 is FLT3 (or vice versa). In some embodiments, TAA 1 is CD133 and TAA 2 is CD52 (or vice versa). In some embodiments, TAA 1 is CD133 and TAA 2 is TNFRSF13C (or vice versa). In some embodiments, TAA 1 is CD133 and TAA 2 is TNFRSF13B (or vice versa). In some embodiments, TAA 1 is CD133 and TAA 2 is CXCR4 (or vice versa). In some embodiments, TAA 1 is CD133 and TAA 2 is PD-L1 (or vice versa). In some embodiments, TAA 1 is CD133 and TAA 2 is LY9 (or vice versa). In some embodiments, TAA 1 is CD133 and TAA 2 is CD200 (or vice versa). In some embodiments, TAA 1 is CD133 and TAA 2 is FCGR2B (or vice versa). In some embodiments, TAA 1 is CD133 and TAA 2 is CD21 (or vice versa). In some embodiments, TAA 1 is CD133 and TAA 2 is CD23 (or vice versa). In some embodiments, TAA 1 is CD133 and TAA 2 is CD24 (or vice versa). In some embodiments, TAA 1 is CD133 and TAA 2 is CD40L (or vice versa). In some embodiments, TAA 1 is CD133 and TAA 2 is CD72 (or vice versa). In some embodiments, TAA 1 is CD133 and TAA 2 is CD79a (or vice versa). In some embodiments, TAA 1 is CD133 and TAA 2 is CD79b (or vice versa). In some embodiments, TAA 1 is FLT3 and TAA 2 is CD52 (or vice versa). In some embodiments, TAA 1 is FLT3 and TAA 2 is TNFRSF13C (or vice versa). In some embodiments, TAA 1 is FLT3 and TAA 2 is TNFRSF13B (or vice versa). In some embodiments, TAA 1 is FLT3 and TAA 2 is CXCR4 (or vice versa). In some embodiments, TAA 1 is FLT3 and TAA 2 is PD-L1 (or vice versa). In some embodiments, TAA 1 is FLT3 and TAA 2 is LY9 (or vice versa). In some embodiments, TAA 1 is FLT3 and TAA 2 is CD200 (or vice versa). In some embodiments, TAA 1 is FLT3 and TAA 2 is FCGR2B (or vice versa). In some embodiments, TAA 1 is FLT3 and TAA 2 is CD21 (or vice versa). In some embodiments, TAA 1 is FLT3 and TAA 2 is CD23 (or vice versa). In some embodiments, TAA 1 is FLT3 and TAA 2 is CD24 (or vice versa). In some embodiments, TAA 1 is FLT3 and TAA 2 is CD40L (or vice versa). In some embodiments, TAA 1 is FLT3 and TAA 2 is CD72 (or vice versa). In some embodiments, TAA 1 is FLT3 and TAA 2 is CD79a (or vice versa). In some embodiments, TAA 1 is FLT3 and TAA 2 is CD79b (or vice versa). In some embodiments, TAA 1 is CD52 and TAA 2 is TNFRSF13C (or vice versa). In some embodiments, TAA 1 is CD52 and TAA 2 is TNFRSF13B (or vice versa). In some embodiments, TAA 1 is CD52 and TAA 2 is CXCR4 (or vice versa). In some embodiments, TAA 1 is CD52 and TAA 2 is PD-L1 (or vice versa). In some embodiments, TAA 1 is CD52 and TAA 2 is LY9 (or vice versa). In some embodiments, TAA 1 is CD52 and TAA 2 is CD200 (or vice versa). In some embodiments, TAA 1 is CD52 and TAA 2 is FCGR2B (or vice versa). In some embodiments, TAA 1 is CD52 and TAA 2 is CD21 (or vice versa). In some embodiments, TAA 1 is CD52 and TAA 2 is CD23 (or vice versa). In some embodiments, TAA 1 is CD52 and TAA 2 is CD24 (or vice versa). In some embodiments, TAA 1 is CD52 and TAA 2 is CD40L (or vice versa). In some embodiments, TAA 1 is CD52 and TAA 2 is CD72 (or vice versa). In some embodiments, TAA 1 is CD52 and TAA 2 is CD79a (or vice versa). In some embodiments, TAA 1 is CD52 and TAA 2 is CD79b (or vice versa). In some embodiments, TAA 1 is TNFRSF13C and TAA 2 is TNFRSF13B (or vice versa). In some embodiments, TAA 1 is TNFRSF13C and TAA 2 is CXCR4 (or vice versa). In some embodiments, TAA 1 is TNFRSF13C and TAA 2 is PD-L1 (or vice versa). In some embodiments, TAA 1 is TNFRSF13C and TAA 2 is LY9 (or vice versa). In some embodiments, TAA 1 is TNFRSF13C and TAA 2 is CD200 (or vice versa). In some embodiments, TAA 1 is TNFRSF13C and TAA 2 is FCGR2B (or vice versa). In some embodiments, TAA 1 is TNFRSF13C and TAA 2 is CD21 (or vice versa). In some embodiments, TAA 1 is TNFRSF13C and TAA 2 is CD23 (or vice versa). In some embodiments, TAA 1 is TNFRSF13C and TAA 2 is CD24 (or vice versa). In some embodiments, TAA 1 is TNFRSF13C and TAA 2 is CD40L (or vice versa). In some embodiments, TAA 1 is TNFRSF13C and TAA 2 is CD72 (or vice versa). In some embodiments, TAA 1 is TNFRSF13C and TAA 2 is CD79a (or vice versa). In some embodiments, TAA 1 is TNFRSF13C and TAA 2 is CD79b (or vice versa). In some embodiments, TAA 1 is TNFRSF13B and TAA 2 is CXCR4 (or vice versa). In some embodiments, TAA 1 is TNFRSF13B and TAA 2 is PD-L1 (or vice versa). In some embodiments, TAA 1 is TNFRSF13B and TAA 2 is LY9 (or vice versa). In some embodiments, TAA 1 is TNFRSF13B and TAA 2 is CD200 (or vice versa). In some embodiments, TAA 1 is TNFRSF13B and TAA 2 is FCGR2B (or vice versa). In some embodiments, TAA 1 is TNFRSF13B and TAA 2 is CD21 (or vice versa). In some embodiments, TAA 1 is TNFRSF13B and TAA 2 is CD23 (or vice versa). In some embodiments, TAA 1 is TNFRSF13B and TAA 2 is CD24 (or vice versa). In some embodiments, TAA 1 is TNFRSF13B and TAA 2 is CD40L (or vice versa). In some embodiments, TAA 1 is TNFRSF13B and TAA 2 is CD72 (or vice versa). In some embodiments, TAA 1 is TNFRSF13B and TAA 2 is CD79a (or vice versa). In some embodiments, TAA 1 is TNFRSF13B and TAA 2 is CD79b (or vice versa). In some embodiments, TAA 1 is CXCR4 and TAA 2 is PD-L1 (or vice versa). In some embodiments, TAA 1 is CXCR4 and TAA 2 is LY9 (or vice versa). In some embodiments, TAA 1 is CXCR4 and TAA 2 is CD200 (or vice versa). In some embodiments, TAA 1 is CXCR4 and TAA 2 is FCGR2B (or vice versa). In some embodiments, TAA 1 is CXCR4 and TAA 2 is CD21 (or vice versa). In some embodiments, TAA 1 is CXCR4 and TAA 2 is CD23 (or vice versa). In some embodiments, TAA 1 is CXCR4 and TAA 2 is CD24 (or vice versa). In some embodiments, TAA 1 is CXCR4 and TAA 2 is CD40L (or vice versa). In some embodiments, TAA 1 is CXCR4 and TAA 2 is CD72 (or vice versa). In some embodiments, TAA 1 is CXCR4 and TAA 2 is CD79a (or vice versa). In some embodiments, TAA 1 is CXCR4 and TAA 2 is CD79b (or vice versa). In some embodiments, TAA 1 is PD-L1 and TAA 2 is LY9 (or vice versa). In some embodiments, TAA 1 is PD-L1 and TAA 2 is CD200 (or vice versa). In some embodiments, TAA 1 is PD-L1 and TAA 2 is FCGR2B (or vice versa). In some embodiments, TAA 1 is PD-L1 and TAA 2 is CD21 (or vice versa). In some embodiments, TAA 1 is PD-L1 and TAA 2 is CD23 (or vice versa). In some embodiments, TAA 1 is PD-L1 and TAA 2 is CD24 (or vice versa). In some embodiments, TAA 1 is PD-L1 and TAA 2 is CD40L (or vice versa). In some embodiments, TAA 1 is PD-L1 and TAA 2 is CD72 (or vice versa). In some embodiments, TAA 1 is PD-L1 and TAA 2 is CD79a (or vice versa). In some embodiments, TAA 1 is PD-L1 and TAA 2 is CD79b (or vice versa). In some embodiments, TAA 1 is LY9 and TAA 2 is CD200 (or vice versa). In some embodiments, TAA 1 is LY9 and TAA 2 is FCGR2B (or vice versa). In some embodiments, TAA 1 is LY9 and TAA 2 is CD21 (or vice versa). In some embodiments, TAA 1 is LY9 and TAA 2 is CD23 (or vice versa). In some embodiments, TAA 1 is LY9 and TAA 2 is CD24 (or vice versa). In some embodiments, TAA 1 is LY9 and TAA 2 is CD40L (or vice versa). In some embodiments, TAA 1 is LY9 and TAA 2 is CD72 (or vice versa). In some embodiments, TAA 1 is LY9 and TAA 2 is CD79a (or vice versa). In some embodiments, TAA 1 is LY9 and TAA 2 is CD79b (or vice versa). In some embodiments, TAA 1 is CD200 and TAA 2 is FCGR2B (or vice versa). In some embodiments, TAA 1 is CD200 and TAA 2 is CD21 (or vice versa). In some embodiments, TAA 1 is CD200 and TAA 2 is CD23 (or vice versa). In some embodiments, TAA 1 is CD200 and TAA 2 is CD24 (or vice versa). In some embodiments, TAA 1 is CD200 and TAA 2 is CD40L (or vice versa). In some embodiments, TAA 1 is CD200 and TAA 2 is CD72 (or vice versa). In some embodiments, TAA 1 is CD200 and TAA 2 is CD79a (or vice versa). In some embodiments, TAA 1 is CD200 and TAA 2 is CD79b (or vice versa). In some embodiments, TAA 1 is CD21 and TAA 2 is CD23 (or vice versa). In some embodiments, TAA 1 is CD21 and TAA 2 is CD24 (or vice versa). In some embodiments, TAA 1 is CD21 and TAA 2 is CD40L (or vice versa). In some embodiments, TAA 1 is CD21 and TAA 2 is CD72 (or vice versa). In some embodiments, TAA 1 is CD21 and TAA 2 is CD79a (or vice versa). In some embodiments, TAA 1 is CD21 and TAA 2 is CD79b (or vice versa). In some embodiments, TAA 1 is CD23 and TAA 2 is CD24 (or vice versa). In some embodiments, TAA 1 is CD23 and TAA 2 is CD40L (or vice versa). In some embodiments, TAA 1 is CD23 and TAA 2 is CD72 (or vice versa). In some embodiments, TAA 1 is CD23 and TAA 2 is CD79a (or vice versa). In some embodiments, TAA 1 is CD23 and TAA 2 is CD79b (or vice versa). In some embodiments, TAA 1 is CD24 and TAA 2 is CD40L (or vice versa). In some embodiments, TAA 1 is CD24 and TAA 2 is CD72 (or vice versa). In some embodiments, TAA 1 is CD24 and TAA 2 is CD79a (or vice versa). In some embodiments, TAA 1 is CD24 and TAA 2 is CD79b (or vice versa). In some embodiments, TAA 1 is CD40L and TAA 2 is CD72 (or vice versa). In some embodiments, TAA 1 is CD40L and TAA 2 is CD79a (or vice versa). In some embodiments, TAA 1 is CD40L and TAA 2 is CD79b (or vice versa). In some embodiments, TAA 1 is CD72 and TAA 2 is CD79a (or vice versa). In some embodiments, TAA 1 is CD72 and TAA 2 is CD79b (or vice versa). In some embodiments, TAA 1 is CD79a and TAA 2 is CD79b (or vice versa).

A TAA-binding ABM can comprise, for example, an anti-TAA antibody or an antigen-binding fragment thereof. The anti-TAA antibody or antigen-binding fragment can comprise, for example, the CDR sequences of an antibody set forth in Table 15A or Table 15B. In some embodiments, the anti-TAA antibody or antigen-binding domain thereof has the heavy and light chain variable region sequences of an antibody set forth in Table 15A. In some embodiments, the anti-TAA antibody or antigen-binding domain thereof has the heavy and light chain variable region sequences of an antibody set forth in Table 15B.

TABLE 15A
Exemplary Anti-Tumor-Associated Antigen Antibodies

| Target | Examples of Antibody Name and/or Reference(s) and/or Source |
|---|---|
| ALK | antibodies described in e.g., Mino-Kenudson et al., 2010, Clin Cancer Res |
| | 16(5): 1561-1571 |
| B7H3 | MGA271 (Macrogenics) |
| BCMA | Any BCMA antibody described in WO2012163805, WO200112812, or |
| | WO2003062401. |
| CAIX | Antibody clone 303123 (R&D Systems) |
| CD123 | U.S. Pat. No. 8,852,551; EP2426148; WO2014138819; WO2016028896; WO2014130635 |
| CD171 | Hong et al., 2014, J Immunother 37(2): 93-104. |
| CD19 | WO2014031687; WO2012079000; WO2014153270; U.S. Pat. No. 7,741,465; the |
| | CD19 binder of Yescarta or Blinatumomab |
| CD20 | Rituximab, Ofatumumab, Ocrelizumab, Veltuzumab, or GA101 |
| CD22 | Haso et al., 2013, Blood, 121(7): 1165-1174; Wayne et al., 2010, Clin Cancer Res |
| | 16(6): 1894-1903; Kato et al., 2013, Leuk Res 37(1): 83-88; Creative BioMart |
| | (creativebiomart.net): MOM-18047-S(P). |
| CD24 | Maliar et al., Gastroenterology 143(5): 1375-1384 (2012) |
| CD30 | Any CD30 antibody described in U.S. Pat. No. 7,090,843 B1, or EP0805871 |
| CD33 | Bross et al., 2001, Clin Cancer Res 7(6): 1490-1496 (Gemtuzumab Ozogamicin, |
| | hP67.6), Caron et al., 1992, Cancer Res 52(24): 6761-6767 (Lintuzumab, HuM195), |
| | Lapusan et al., 2012, Invest New Drugs 30(3): 1121-1131 (AVE9633), Aigner et al., |
| | 2013, Leukemia 27(5): 1107-1115 (AMG330, CD33 BiTE), Dutour et al., 2012, Adv |
| | Hematol 2012: 683065, or Pizzitola et al., 2014, Leukemia |
| | doi: 10.1038/Lue.2014.62. |
| CD38 | Daratumumab (see, e.g., Groen et al., 2010, Blood 116(21): 1261-1262; MOR202 |
| | (see, e.g., U.S. Pat. No. 8,263,746); or any CD38 antibody described in U.S. Pat. No. |
| | 8,362,211. |
| CD44v6 | Casucci et al., 2013, Blood 122(20): 3461-3472. |
| CD97 | antibodies described in, e.g., U.S. Pat. No. 6,846,911; de Groot et al., 2009, J |
| | Immunol 183(6): 4127-4134; antibody from R&D: MAB373 |
| CEA | Chmielewski et al., 2012, Gastoenterology 143(4): 1095-1107. |
| CLDN6 | WO2015069794; IMAB027, mAb, Ganymed Pharmaceuticals |
| CLL-1 | PE-CLL1-hu Cat# 353604 (BioLegend); and PE-CLL1 (CLEC12A) Cat# 562566 |
| | (BD); WO 2014/051433 A1; US 2016/0368994 A1; US 2013/0295118 A1; U.S. Pat. |
| | No. 8,536,310 B2; Lu et al., 2014, Angewandte Chemie International Edition |
| | 53(37): 9841-9845; Leong et al., 2017, Blood 129(5): 609-618 |
| CS1 | Elotuzumab (BMS), see e.g., Tai et al., 2008, Blood 112(4): 1329-37; Tai et al., |
| | 2007, Blood. 110(5): 1656-63. |
| EGFR | Cetuximab, panitumumab, zalutumumab, nimotuzumab, or matuzumab |
| EGFRvIII | WO2012138475; WO2014130657 |
| EPCAM | MT110, EpCAM-CD3 bispecific Ab (see, e.g., |
| | clinicaltrials.gov/ct2/show/NCT00635596); Edrecolomab; 3622W94; ING-1; or |
| | adecatumumab (MT201). |
| EphA2 | Yu et al., 2014, Mol Ther 22(1): 102-111. |
| Ephrin B2 | Abengozar et al., 2012, Blood 119(19): 4565-4576. |
| ERBB2 | Trastuzumab or pertuzumab. |
| (Her2/neu) | |
| FAP | Ostermann et al., 2008, Clinical Cancer Research 14: 4584-4592 (FAP5), US Pat. |
| | Publication No. 2009/0304718; sibrotuzumab (see e.g., Hofheinz et al., 2003, |
| | Oncology Research and Treatment 26(1): 44-48); and Tran et al., 2013, J Exp Med |
| | 210(6): 1125-1135. |
| FLT3 | Any FLT3 antibody described in WO2011076922, U.S. Pat. No. 5,777,084, |
| | EP0754230, or US20090297529. |
| Folate | IMGN853, or any folate receptor alpha antibody described in US20120009181; U.S. |
| receptor | Pat. No. 4,851,332, LK26: U.S. Pat. No. 5,952,484. |
| alpha | |
| Folate | antibodies described in, e.g., US20100297138; WO2007/067992 |
| receptor | |
| beta | |
| GD2 | Mujoo et al., Cancer Res. 47(4): 1098-1104 (1987); Cheung et al., Cancer Res |
| | 45(6): 2642-2649 (1985), Cheung et al., J Clin Oncol 5(9): 1430-1440 (1987), |
| | Cheung et al., J Clin Oncol 16(9): 3053-3060 (1998), Handgretinger et al., Cancer |
| | Immunol Immunother 35(3): 199-204 (1992); |
| | mAb 14.18, 14G2a, ch14.18, hu14.18, 3F8, hu3F8, 3G6, 8B6, 60C3, 10B8, |
| | ME36.1, or 8H9 (see e.g., WO2012033885, WO2013040371, WO2013192294, |
| | WO2013061273, WO2013123061, WO2013074916, and WO201385552). |
| | Any GD2 antibody described in US Publication No.: 20100150910 or PCT |
| | Publication No.: WO 2011160119. |
| GD3 | Any GD3 antibody described in U.S. Pat. No. 7,253,263; U.S. Pat. No. 8,207,308; US |
| | 20120276046; EP1013761; W02005035577; or U.S. Pat. No. 6,437,098. |
| GloboH | VK9; Kudryashov et al., 1998, Glycoconj J. 15(3): 243-9; Lou et al., 2014, Proc Natl |
| | Acad Sci USA 111(7): 2482-2487; MBr1: Bremer et al., 1984, J Biol Chem |
| | 259: 14773-14777. |
| gp100 | HMB45, NKIbetaB, or any anti-gp100 antibody described in WO2013165940, or |
| | US20130295007 |
| GPRC5D | R&Dsystems: FAB6300A; Lifespan Biosciences: LS-A4180 |
| HMWMAA | antibodies described in, e.g., Kmiecik et al., 2014, Oncoimmunology 3(1): e27185 |
| | (PMID: 24575382) (mAb9.2.27); U.S. Pat. No. 6,528,481; WO2010033866; US |
| | 20140004124 |
| IGF-I | Any IGF-I receptor antibody described in U.S. Pat. No. 8,344,112 B2; EP2322550 A1; WO |
| receptor | 2006/138315, or PCT/US2006/022995. |
| IL-11Ra | Abcam (cat# ab55262) or Novus Biologicals (cat# EPR5446) |
| IL-13Ra2 | Any IL-13Ra2 antibody described in WO2008/146911, WO2004087758, or |
| | WO2004087758 |
| KIT | Any KIT antibody described in U.S. Pat. No. 7,915,391, US20120288506 |
| KLRG2 | ab121563 (Abcam); B-12 or sc-514346 (Santa Cruz); HPA018199 (Sigma Aldrich) |
| LewisY | Kelly et al., Cancer Biother Radiopharm 23(4): 411-423 (2008) (hu3S193 Ab |
| | (scFvs)); Dolezal et al., Protein Engineering 16(1): 47-56 (2003) (NC10 scFv) |
| LMP2 | Any LMP2 antibody described in U.S. Pat. No. 7,410,640 or US 2005/0129701 |
| LRP6 | WO2009064944, WO2009056634, WO2011119661, WO2011138392, |
| | WO2011138391, WO2013067355, WO2014029752, WO2017093478 |
| Mesothelin | Any mesothelin antibody described in US 20110262448, US 2012/0107933 or |
| | U.S. Pat. No. 9,719,996 |
| MUC1 | SAR566658 |
| NCAM | 2-2B: MAB5324 (EMD Millipore) |
| NY-BR-1 | antibodies described in, e.g., Jager et al., 2007, Appl Immunohitochem Mol |
| | Morphol 15(1): 77-83 |
| o-acetyl- | 8B6 |
| GD2 | |
| PDGFR- | Abcam ab32570 |
| beta | |
| PLAC1 | antibodies described in, e.g., Ghods et al., 2013, Biotechnol Appl Biochem |
| | doi: 10.1002/bab.1177 |
| Polysialic | antibodies described in e.g., Nagae et al., 2013, J Biol Chem 288(47): 33784- |
| acid | 33796 |
| PRSS21 | Any PRSS21 antibody described in U.S. Pat. No. 8,080,650. |
| PSCA | Morgenroth et al., Prostate 67(10): 1121-1131 (2007) (scFv 7F5); Nejatollahi et al., |
| | J of Oncology 2013(2013), article ID 839831 (scFv C5-II); or any PSCA antibody |
| | described in US Pat Publication No. 20090311181. |
| PSMA | Parker et al., Protein Expr Purif 89(2): 136-145 (2013), US 20110268656 (J591 |
| | ScFv); Frigerio et al, European J Cancer 49(9): 2223-2232 (2013) (scFvD2B); WO |
| | 2006125481 (mAbs 3/A12, 3/E7 and 3/F11) or single chain antibody fragments |
| | (scFv A5 and D7). |
| ROR1 | Hudecek et al., Clin Cancer Res 19(12): 3153-3164 (2013); or any ROR1 antibody |
| | described in WO 2011159847 or US20130101607. |
| SSEA-4 | MC813 (Cell Signaling) |
| TAG72 | Hornbach et al., Gastroenterology 113(4): 1163-1170 (1997) or Abcam ab691. |
| TEM1/ | antibodies described in, e.g., Marty et al., 2006, Cancer Lett 235(2): 298-308; Zhao |
| CD248 | et al., 2011, J Immunol Methods 363(2): 221-232 |
| Tn | Brooks et al., PNAS 107(22): 10056-10061 (2010); Stone et al., Oncolmmunology |
| | 1(6): 863-873(2012); any Tn antibody described in U.S. Pat. No. 8,440,798 |
| TSHR | antibodies described in, e.g., Marty et al., 2006, Cancer Lett 235(2): 298-308; Zhao |
| | et al., 2011, J Immunol Methods 363(2): 221-232 |
| Tyrosinase | Any tyrosinase antibody described in U.S. Pat. No. 5,843,674 or US19950504048. |
| VEGFR2 | Chinnasamy et al., J Clin Invest 120(11): 3953-3968 (2010). |

TABLE 15B
Exemplary Anti-Tumor-Associated Antigen Antibodies

| Target | Examples of Antibody Name and/or Reference(s) and/or Source |
|---|---|
| CD123 | Any CD123 antibody described in U.S. Pat. No. 8,852,551, EP2426148, WO |
| | 2014/138819, WO 2016/028896, or WO 2014/130635 |
| BCMA | Any BCMA antibody described in WO2012163805, WO200112812, or |
| | WO2003062401. |
| CD19 | Any CD19 antibody described in WO 2014/031687, WO 2012/079000, WO |
| | 2014/153270, or U.S. Pat. No. 7,741,465; the CD19 binder of Yescarta or |
| | Blinatumomab |
| CD20 | Rituximab, Ofatumumab, Ocrelizumab, Veltuzumab, or GA101 |
| CD22 | Any CD22 antibody described in Haso et al., 2013, Blood, 121(7): 1165-1174, |
| | Wayne et al., 2010, Clin Cancer Res 16(6): 1894-1903, Kato et al., 2013, Leuk |
| | Res 37(1): 83-88, or Creative BioMart (creativebiomart.net): MOM-18047-S(P). |
| CD33 | Any CD33 antibody described in Bross et al., 2001, Clin Cancer Res 7(6): 1490- |
| | 1496 (Gemtuzumab Ozogamicin, hP67.6),Caron et al., 1992, Cancer Res |
| | 52(24): 6761-6767 (Lintuzumab, HuM195), Lapusan et al., 2012, Invest New |
| | Drugs 30(3): 1121-1131 (AVE9633), Aigner et al., 2013, Leukemia 27(5): 1107- |
| | 1115 (AMG330, CD33 BiTE), Dutour et al., 2012, Adv Hematol 2012: 683065, or |
| | Pizzitola et al., 2014, Leukemia doi: 10.1038/Lue.2014.62. |
| CD38 | Daratumumab (see, e.g., Groen et al., 2010, Blood 116(21): 1261-1262; MOR202 |
| | (see, e.g., U.S. Pat. No. 8,263,746); or any CD38 antibody described in U.S. Pat. |
| | No. 8,362,211. |
| CLL-1 | PE-CLL1-hu Cat# 353604 (BioLegend); PE-CLL1 (CLEC12A) Cat# 562566 (BD); |
| | Any CLL-1 antibody described in WO 2014/051433 A1, US 2016/0368994 A1, |
| | US 2013/0295118 A1, U.S. Pat. No. 8,536,310 B2, Lu et al., 2014, Angewandte |
| | Chemie International Edition 53(37): 9841-9845, or Leong et al., 2017, Blood |
| | 129(5): 609-618 |
| CS1 | Elotuzumab (BMS), see e.g., Tai et al., 2008, Blood 112(4): 1329-37; Tai et al., |
| | 2007, Blood. 110(5): 1656-63. |
| FLT3 | Any FLT3 antibody described in WO 2011/076922, U.S. Pat. No. 5,777,084, |
| | EP0754230, or US 2009/0297529. |
| CD133 | Any CD133 antibody described in U.S. Pat. No. 9,624,303, WO 2016/154623, or |
| | WO 2011/089211; 5E3 (ThermoFisher); MAB11331 (R&D Systems); MAB4310 |
| | (Millipore Sigma) |
| CD138 | Any CD138 antibody described in WO/2009/080829, WO/2017/014679, or U.S. |
| | Pat. No. 9,289,509; nBT062 (Biotest AG); MI15, B-A38, SP152, DL-101 |
| | (ThermoFisher) |
| CD52 | alemtuzumab (Genzyme); ANT1034 (see, Holgate et al., 2015, PLOS ONE 10(9): |
| | e0138123; any CD52 antibody described in WO/2010/132659; any CD52 |
| | antibody described in U.S. Pat. No. 9,708,407; any CD52 antibody described in |
| | WO/2010/132659 |
| TNFRSF13C | Any TNFRSF13C antibody described in WO 2010/007082, U.S. Pat. No. |
| | 9,382,326 |
| TNFRSF13B | Any TNFRSF13B antibody described in WO 2004/011611; LS-C89973 (Lifespan |
| | Biosciences, Inc.) M02952-1 (Boster Biological Technology); MAB1041, |
| | MAB1741, and MAB174 (R&D Systems) |
| CXCR4 | Any CXCR4 antibody described in U.S. Pat. Nos. 7,138,496, 8,329,178, |
| | 8,450,464, 9,249,223, or 9,260,527 |
| PD-L1 | Any PD-L1 antibody described in US 2015/0203580, US 2017/0058033, US |
| | 2017/0204184, U.S. Pat. No. 8,741,295, U.S. Pat. No. 9,789,183, or U.S. Pat. No. |
| | 9,637,546 |
| LY9 | HLy9.25 (e.g., Lifespan Biosciences, Inc. cat. no. LS-C112605); MAB1898 (R&D |
| | Systems) |
| CD200 | Any CD200 antibody described in U.S. Pat. No. 7,887,798; ab23552 (Abcam); |
| | Ox104 (ThermoFisher) |
| FCGR2B | Any FCGR2B antibody described in U.S. Pat. No. 8,802,089 or WO 2017/103895; |
| | ab45143 (Abcam); AT130-2 (ThermoFisher); 2E10 (Millipore Sigma) |
| CD21 | ab75985 (Abcam); ab9492 (Abcam); 2G9 (ThermoFisher); HB5 (ThermoFisher); |
| | MAB4909 (R&D Systems) |
| CD23 | Any CD23 antibody described in U.S. Pat. No. 7,008,623 or U.S. Pat. No. |
| | 6,011,138; lumiliximab (Biogen); ab16702 (Abcam); SP23 (ThermoFisher) |
| CD24 | Any CD24 antibody described in U.S. Pat. No. 8,614,301; SN3 (ThermoFisher); |
| | SN3b (ThermoFisher); 2Q1282 (Santa Cruz Biotechnology); 3H1143 (Santa Cruz |
| | Biotechnology); ALB9 (Santa Cruz Biotechnology); MAB5248 (R&D Systems) |
| CD40L | Any CD40L antibody described in U.S. Pat. No. 9,228,018 or US 2003/0099642; |
| | 24-31 (Biolegend); ab52750 (Abcam); ab47204 (Abcam); CDP7657 (UCB |
| | Pharma); 5c8 (Biogen) |
| CD72 | 3F3 (Biolegend); Bu40 (ThermoFisher); H-7 (Santa Cruz Biotechnology); H-96 |
| | (Santa Cruz Biotechnology); G-5 (Santa Cruz Biotechnology); ab92509 (Abcam) |
| CD79a | ab62650 (Abcam); ab79414 (Abcam); MAB69201 (R&D Systems); HM57 (Bio- |
| | Rad) |
| CD79b | Any CD79b antibody described in WO 2014/011521; ab130422 (Abcam); |
| | ab134147 (Abcam); polatuzumab (Genentech) |

In certain embodiments, TAA 1 and TAA 2 are selected from CD19, CD20 and BCMA. In other embodiments, TAA 1 and TAA 2 are selected from BCMA and CD19. Exemplary BCMA and CD19 binding sequences are set forth in Sections 7.10.1 and 7.10.2, infra.

7.10.1. BCMA

In certain aspects, the present disclosure provides a MBM in which ABM2 or ABM3 is BCMA (such ABMs can be referred to as “BCMA ABMs” for convenience). BCMA is a tumor necrosis family receptor (TNFR) member expressed on cells of the B-cell lineage. BCMA expression is the highest on terminally differentiated B cells that assume the long lived plasma cell fate, including plasma cells, plasmablasts and a subpopulation of activated B cells and memory B cells. BCMA is involved in mediating the survival of plasma cells for maintaining long-term humoral immunity. The expression of BCMA has been recently linked to a number of cancers, autoimmune disorders, and infectious diseases. Cancers with increased expression of BCMA include some hematological cancers, such as multiple myeloma, Hodgkin's and non-Hodgkin's lymphoma, various leukemias, and glioblastoma.

MBMs comprising an ABM that binds to BCMA can comprise, for example, an anti-BCMA antibody or an antigen-binding domain thereof. The anti-BCMA antibody or antigen-binding domain thereof can comprise, for example, CDR, VH, VL, or scFV sequences set forth in Tables 16A-16G.

TABLE 16A
BCMA Binders - Variable domain and scFv sequences

| | | | SEQ |
|---|---|---|---|
| | | | ID |
| Antibody | Domain | Sequence | NO. |
| BCMA-1 | VH | EVQLVESGGGLVQPGGSLRLSCAVSGFALSNHGMSVVVRRAPGK | 687 |
| | | GLEVVVSGIVYSGSTYYAASVKGRFTISRDNSRNTLYLQMNSLRPE | |
| | | DTAIYYCSAHGGESDVWGQGTTVTVSS | |
| | VL | DIQLTQSPSSLSASVGDRVTITCRASQSISSYLNVVYQQKPGKAPKL | 688 |
| | | LIYAASSLQSGVPSRFSGSGSGTDFTLTISSLQPEDFATYYCQQSY | |
| | | STPYTFGQGTKVEIK | |
| | scFv | EVQLVESGGGLVQPGGSLRLSCAVSGFALSNHGMSVVVRRAPGK | 689 |
| | | GLEVVVSGIVYSGSTYYAASVKGRFTISRDNSRNTLYLQMNSLRPE | |
| | | DTAIYYCSAHGGESDVWGQGTTVTVSSASGGGGSGGRASGGGG | |
| | | SDIQLTQSPSSLSASVGDRVTITCRASQSISSYLNVVYQQKPGKAPK | |
| | | LLIYAASSLQSGVPSRFSGSGSGTDFTLTISSLQPEDFATYYCQQS | |
| | | YSTPYTFGQGTKVEIK | |
| BCMA-2 | VH | QVQLVESGGGLVQPGRSLRLSCAASGFTFSNYAMSVVVRQAPGK | 690 |
| | | GLGVVVSGISRSGENTYYADSVKGRFTISRDNSKNTLYLQMNSLRD | |
| | | EDTAVYYCARSPAHYYGGMDVWGQGTTVTVSS | |
| | VL | DIVLTQSPGTLSLSPGERATLSCRASQSISSSFLAVVYQQKPGQAP | 691 |
| | | RLLIYGASRRATGIPDRFSGSGSGTDFTLTISRLEPEDSAVYYCQQ | |
| | | YHSSPSVVTFGQGTKLEIK | |
| | scFv | QVQLVESGGGLVQPGRSLRLSCAASGFTFSNYAMSVVVRQAPGK | 692 |
| | | GLGVVVSGISRSGENTYYADSVKGRFTISRDNSKNTLYLQMNSLRD | |
| | | EDTAVYYCARSPAHYYGGMDVWGQGTTVTVSSASGGGGSGGRA | |
| | | SGGGGSDIVLTQSPGTLSLSPGERATLSCRASQSISSSFLAVVYQQ | |
| | | KPGQAPRLLIYGASRRATGIPDRFSGSGSGTDFTLTISRLEPEDSA | |
| | | VYYCQQYHSSPSVVTFGQGTKLEIK | |
| BCMA-3 | VH | QVQLVESGGGLVQPGRSLRLSCAASGFTFDDYAMHVVVRQAPGK | 693 |
| | | GLEVVVSGISWNSGSIGYADSVKGRFTISRDNAKNSLYLQMNSLRA | |
| | | EDTALYYCSVHSFLAYWGQGTLVTVSS | |
| | VL | DIVMTQTPLSLPVTPGEPASISCRSSQSLLHSNGYNYLDVVYLQKP | 694 |
| | | GQSPQLLIYLGSNRASGVPDRFSGSGSGTDFTLKISRVEAEDVGV | |
| | | YYCMQALQTPYTFGQGTKVEIK | |
| | scFv | QVQLVESGGGLVQPGRSLRLSCAASGFTFDDYAMHVVVRQAPGK | 695 |
| | | GLEVVVSGISWNSGSIGYADSVKGRFTISRDNAKNSLYLQMNSLRA | |
| | | EDTALYYCSVHSFLAYWGQGTLVTVSSASGGGGSGGRASGGGG | |
| | | SDIVMTQTPLSLPVTPGEPASISCRSSQSLLHSNGYNYLDVVYLQKP | |
| | | GQSPQLLIYLGSNRASGVPDRFSGSGSGTDFTLKISRVEAEDVGV | |
| | | YYCMQALQTPYTFGQGTKVEIK | |
| BCMA-4 | VH | EVQLLESGGGLVQPGGSLRLSCAVSGFALSNHGMSVVVRRAPGK | 696 |
| | | GLEVVVSGIVYSGSTYYAASVKGRFTISRDNSRNTLYLQMNSLRPE | |
| | | DTAIYYCSAHGGESDVWGQGTTVTVSS | |
| | VL | DIVMTQTPLSLSVTPGQPASISCKSSQSLLRNDGKTPLYVVYLQKA | 697 |
| | | GQPPQLLIYEVSNRFSGVPDRFSGSGSGTDFTLKISRVEAEDVGA | |
| | | YYCMQNIQFPSFGGGTKLEIK | |
| | scFv | EVQLLESGGGLVQPGGSLRLSCAVSGFALSNHGMSVVVRRAPGK | 698 |
| | | GLEVVVSGIVYSGSTYYAASVKGRFTISRDNSRNTLYLQMNSLRPE | |
| | | DTAIYYCSAHGGESDVWGQGTTVTVSSASGGGGSGGRASGGGG | |
| | | SDIVMTQTPLSLSVTPGQPASISCKSSQSLLRNDGKTPLYVVYLQKA | |
| | | GQPPQLLIYEVSNRFSGVPDRFSGSGSGTDFTLKISRVEAEDVGA | |
| | | YYCMQNIQFPSFGGGTKLEIK | |
| BCMA-5 | VH | QVQLVQSGAEVRKTGASVKVSCKASGYIFDNFGINVVVRQAPGQG | 699 |
| | | LEWMGWINPKNNNTNYAQKFQGRVTITADESTNTAYMEVSSLRS | |
| | | EDTAVYYCARGPYYYQSYMDVWGQGTMVTVSS | |
| | VL | DIVMTQTPLSLPVTPGEPASISCRSSQSLLHSNGYNYLNVVYLQKP | 700 |
| | | GQSPQLLIYLGSKRASGVPDRFSGSGSGTDFTLHITRVGAEDVGV | |
| | | YYCMQALQTPYTFGQGTKLEIK | |
| | scFv | QVQLVQSGAEVRKTGASVKVSCKASGYIFDNFGINVVVRQAPGQG | 701 |
| | | LEWMGWINPKNNNTNYAQKFQGRVTITADESTNTAYMEVSSLRS | |
| | | EDTAVYYCARGPYYYQSYMDVWGQGTMVTVSSASGGGGSGGR | |
| | | ASGGGGSDIVMTQTPLSLPVTPGEPASISCRSSQSLLHSNGYNYL | |
| | | NVVYLQKPGQSPQLLIYLGSKRASGVPDRFSGSGSGTDFTLHITRV | |
| | | GAEDVGVYYCMQALQTPYTFGQGTKLEIK | |
| BCMA-6 | VH | QVQLQESGGGLVQPGGSLRLSCAASGFTFSSDAMTVVVRQAPGK | 702 |
| | | GLEVVVSVISGSGGTTYYADSVKGRFTISRDNSKNTLYLQMNSLRA | |
| | | EDTAVYYCAKLDSSGYYYARGPRYWGQGTLVTVSS | |
| | VL | DIQLTQSPSSLSASVGDRVTITCRASQSISSYLNVVYQQKPGKAPKL | 703 |
| | | LIYGASTLASGVPARFSGSGSGTHFTLTINSLQSEDSATYYCQQSY | |
| | | KRASFGQGTKVEIK | |
| | scFv | QVQLQESGGGLVQPGGSLRLSCAASGFTFSSDAMTVVVRQAPGK | 704 |
| | | GLEVVVSVISGSGGTTYYADSVKGRFTISRDNSKNTLYLQMNSLRA | |
| | | EDTAVYYCAKLDSSGYYYARGPRYWGQGTLVTVSSASGGGGSG | |
| | | GRASGGGGSDIQLTQSPSSLSASVGDRVTITCRASQSISSYLNVVY | |
| | | QQKPGKAPKLLIYGASTLASGVPARFSGSGSGTHFTLTINSLQSED | |
| | | SATYYCQQSYKRASFGQGTKVEIK | |
| BCMA-7 | VH | QVQLVQSGAEVKKPGASVKVSCKASGYTFSNYGITVVVRQAPGQG | 705 |
| | | LEWMGWISAYNGNTNYAQKFQGRVTMTRNTSISTAYMELSSLRS | |
| | | EDTAVYYCARGPYYYYMDVWGKGTMVTVSS | |
| | VL | EIVMTQSPLSLPVTPGEPASISCRSSQSLLYSNGYNYVDVVYLQKP | 706 |
| | | GQSPQLLIYLGSNRASGVPDRFSGSGSGTDFKLQISRVEAEDVGIY | |
| | | YCMQGRQFPYSFGQGTKVEIK | |
| | scFv | QVQLVQSGAEVKKPGASVKVSCKASGYTFSNYGITVVVRQAPGQG | 707 |
| | | LEWMGWISAYNGNTNYAQKFQGRVTMTRNTSISTAYMELSSLRS | |
| | | EDTAVYYCARGPYYYYMDVWGKGTMVTVSSASGGGGSGGRAS | |
| | | GGGGSEIVMTQSPLSLPVTPGEPASISCRSSQSLLYSNGYNYVDW | |
| | | YLQKPGQSPQLLIYLGSNRASGVPDRFSGSGSGTDFKLQISRVEA | |
| | | EDVGIYYCMQGRQFPYSFGQGTKVEIK | |
| BCMA-8 | VH | EVQLLETGGGLVQPGGSLRLSCAVSGFALSNHGMSVVVRRAPGK | 708 |
| | | GLEVVVSGIVYSGSTYYAASVKGRFTISRDNSRNTLYLQMNSLRPE | |
| | | DTAIYYCSAHGGESDVWGQGTTVTVSS | |
| | VL | EIVLTQSPATLSVSPGESATLSCRASQSVSSNLAVVYQQKPGQAPR | 709 |
| | | LLIYGASTRASGIPDRFSGSGSGTDFTLTISSLQAEDVAVYYCQQY | |
| | | GSSLTFGGGTKVEIK | |
| | scFv | EVQLLETGGGLVQPGGSLRLSCAVSGFALSNHGMSVVVRRAPGK | 710 |
| | | GLEVVVSGIVYSGSTYYAASVKGRFTISRDNSRNTLYLQMNSLRPE | |
| | | DTAIYYCSAHGGESDVWGQGTTVTVSSASGGGGSGGRASGGGG | |
| | | SEIVLTQSPATLSVSPGESATLSCRASQSVSSNLAVVYQQKPGQAP | |
| | | RLLIYGASTRASGIPDRFSGSGSGTDFTLTISSLQAEDVAVYYCQQ | |
| | | YGSSLTFGGGTKVEIK | |
| BCMA-9 | VH | EVQLVETGGGLVQPGGSLRLSCAVSGFALSNHGMSVVVRRAPGK | 711 |
| | | GLEVVVSGIVYSGSTYYAASVKGRFTISRDNSRNTLYLQMNSLRPE | |
| | | DTAIYYCSAHGGESDVWGQGTTVTVSS | |
| | VL | EIVMTQSPATLSVSPGERATLSCRASQSVSSKLAVVYQQKPGQAP | 712 |
| | | RLLMYGASIRATGIPDRFSGSGSGTEFTLTISSLEPEDFAVYYCQQ | |
| | | YGSSSVVTFGQGTKVEIK | |
| | scFv | EVQLVETGGGLVQPGGSLRLSCAVSGFALSNHGMSVVVRRAPGK | 713 |
| | | GLEVVVSGIVYSGSTYYAASVKGRFTISRDNSRNTLYLQMNSLRPE | |
| | | DTAIYYCSAHGGESDVWGQGTTVTVSSASGGGGSGGRASGGGG | |
| | | SEIVMTQSPATLSVSPGERATLSCRASQSVSSKLAVVYQQKPGQA | |
| | | PRLLMYGASIRATGIPDRFSGSGSGTEFTLTISSLEPEDFAVYYCQ | |
| | | QYGSSSVVTFGQGTKVEIK | |
| BCMA-10 | VH | EVQLVETGGGVVQPGGSLRLSCAVSGFALSNHGMSVVVRRAPGK | 714 |
| | | GLEVVVSGIVYSGSTYYAASVKGRFTISRDNSRNTLYLQMNSLRPE | |
| | | DTAIYYCSAHGGESDVWGQGTTVTVSS | |
| | VL | EIVLTQSPGTLSLSPGERATLSCRASQSVGSTNLAVVYQQKPGQAP | 715 |
| | | RLLIYDASNRATGIPDRFSGGGSGTDFTLTISRLEPEDFAVYYCQQ | |
| | | YGSSPPVVTFGQGTKVEIK | |
| | scFv | EVQLVETGGGVVQPGGSLRLSCAVSGFALSNHGMSVVVRRAPGK | 716 |
| | | GLEVVVSGIVYSGSTYYAASVKGRFTISRDNSRNTLYLQMNSLRPE | |
| | | DTAIYYCSAHGGESDVWGQGTTVTVSSASGGGGSGGRASGGGG | |
| | | SEIVLTQSPGTLSLSPGERATLSCRASQSVGSTNLAVVYQQKPGQA | |
| | | PRLLIYDASNRATGIPDRFSGGGSGTDFTLTISRLEPEDFAVYYCQ | |
| | | QYGSSPPVVTFGQGTKVEIK | |
| BCMA-11 | VH | QVQLVESGGGLVKPGGSLRLSCAASGFTFSDYYMSWIRQAPGKG | 717 |
| | | LEVVVSYISSSGSTIYYADSVKGRFTISRDNAKNSLYLQMNSLRAED | |
| | | TAVYYCARESGDGMDVWGQGTTVTVSS | |
| | VL | DIQMTQSPSSLSASVGDRVTITCRASQSISSYLNVVYQQKPGKAPK | 718 |
| | | LLIYAASSLQSGVPSRFSGSGSGTDFTLTISSLQPEDFATYYCQQS | |
| | | YTLAFGQGTKVDIK | |
| | scFv | QVQLVESGGGLVKPGGSLRLSCAASGFTFSDYYMSWIRQAPGKG | 719 |
| | | LEVVVSYISSSGSTIYYADSVKGRFTISRDNAKNSLYLQMNSLRAED | |
| | | TAVYYCARESGDGMDVWGQGTTVTVSSASGGGGSGGRASGGG | |
| | | GSDIQMTQSPSSLSASVGDRVTITCRASQSISSYLNVVYQQKPGKA | |
| | | PKLLIYAASSLQSGVPSRFSGSGSGTDFTLTISSLQPEDFATYYCQ | |
| | | QSYTLAFGQGTKVDIK | |
| BCMA-12 | VH | QVQLVQSGGGLVKPGGSLRLSCAASGFTFSDYYMSWIRQAPGKG | 720 |
| | | LEVVVSYISSSGNTIYYADSVKGRFTISRDNAKNSLYLQMNSLRAED | |
| | | TAVYYCARSTMVREDYWGQGTLVTVSS | |
| | VL | DIVLTQSPLSLPVTLGQPASISCKSSESLVHNSGKTYLNWFHQRPG | 721 |
| | | QSPRRLIYEVSNRDSGVPDRFTGSGSGTDFTLKISRVEAEDVGVY | |
| | | YCMQGTHVVPGTFGQGTKLEIK | |
| | scFv | QVQLVQSGGGLVKPGGSLRLSCAASGFTFSDYYMSWIRQAPGKG | 722 |
| | | LEVVVSYISSSGNTIYYADSVKGRFTISRDNAKNSLYLQMNSLRAED | |
| | | TAVYYCARSTMVREDYWGQGTLVTVSSASGGGGSGGRASGGG | |
| | | GSDIVLTQSPLSLPVTLGQPASISCKSSESLVHNSGKTYLNVVFHQR | |
| | | PGQSPRRLIYEVSNRDSGVPDRFTGSGSGTDFTLKISRVEAEDVG | |
| | | VYYCMQGTHVVPGTFGQGTKLEIK | |
| BCMA-13 | VH | QVQLVESGGGLVQPGGSLRLSCAVSGFALSNHGMSVVVRRAPGK | 723 |
| | | GLEVVVSGIVYSGSTYYAASVKGRFTISRDNSRNTLYLQMNSLRPE | |
| | | DTAIYYCSAHGGESDVWGQGTTVTVSS | |
| | VL | DIRLTQSPSPLSASVGDRVTITCQASEDINKFLNVVYHQTPGKAPKL | 724 |
| | | LIYDASTLQTGVPSRFSGSGSGTDFTLTINSLQPEDIGTYYCQQYE | |
| | | SLPLTFGGGTKVEIK | |
| | scFv | QVQLVESGGGLVQPGGSLRLSCAVSGFALSNHGMSVVVRRAPGK | 725 |
| | | GLEVVVSGIVYSGSTYYAASVKGRFTISRDNSRNTLYLQMNSLRPE | |
| | | DTAIYYCSAHGGESDVWGQGTTVTVSSASGGGGSGGRASGGGG | |
| | | SDIRLTQSPSPLSASVGDRVTITCQASEDINKFLNVVYHQTPGKAPK | |
| | | LLIYDASTLQTGVPSRFSGSGSGTDFTLTINSLQPEDIGTYYCQQY | |
| | | ESLPLTFGGGTKVEIK | |
| BCMA-14 | VH | EVQLVETGGGLVQPGGSLRLSCAVSGFALSNHGMSVVVRRAPGK | 711 |
| | | GLEVVVSGIVYSGSTYYAASVKGRFTISRDNSRNTLYLQMNSLRPE | |
| | | DTAIYYCSAHGGESDVWGQGTTVTVSS | |
| | VL | ETTLTQSPATLSVSPGERATLSCRASQSVGSNLAVVYQQKPGQGP | 726 |
| | | RLLIYGASTRATGIPARFSGSGSGTEFTLTISSLQPEDFAVYYCQQY | |
| | | NDWLPVTFGQGTKVEIK | |
| | scFv | EVQLVETGGGLVQPGGSLRLSCAVSGFALSNHGMSVVVRRAPGK | 727 |
| | | GLEVVVSGIVYSGSTYYAASVKGRFTISRDNSRNTLYLQMNSLRPE | |
| | | DTAIYYCSAHGGESDVWGQGTTVTVSSASGGGGSGGRASGGGG | |
| | | SETTLTQSPATLSVSPGERATLSCRASQSVGSNLAVVYQQKPGQG | |
| | | PRLLIYGASTRATGIPARFSGSGSGTEFTLTISSLQPEDFAVYYCQQ | |
| | | YNDWLPVTFGQGTKVEIK | |
| BCMA-15 | VH | EVQLVESGGGLVQPGGSLRLSCAVSGFALSNHGMSVVVRRAPGK | 687 |
| | | GLEVVVSGIVYSGSTYYAASVKGRFTISRDNSRNTLYLQMNSLRPE | |
| | | DTAIYYCSAHGGESDVWGQGTTVTVSS | |
| | VL | EIVLTQSPGTLSLSPGERATLSCRASQSIGSSSLAVVYQQKPGQAP | 728 |
| | | RLLMYGASSRASGIPDRFSGSGSGTDFTLTISRLEPEDFAVYYCQ | |
| | | QYAGSPPFTFGQGTKVEIK | |
| | scFv | EVQLVESGGGLVQPGGSLRLSCAVSGFALSNHGMSVVVRRAPGK | 729 |
| | | GLEVVVSGIVYSGSTYYAASVKGRFTISRDNSRNTLYLQMNSLRPE | |
| | | DTAIYYCSAHGGESDVWGQGTTVTVSSASGGGGSGGRASGGGG | |
| | | SEIVLTQSPGTLSLSPGERATLSCRASQSIGSSSLAVVYQQKPGQA | |
| | | PRLLMYGASSRASGIPDRFSGSGSGTDFTLTISRLEPEDFAVYYCQ | |
| | | QYAGSPPFTFGQGTKVEIK | |
| BCMA-16 | VH | QVQLQESGPGLVKPSETLSLTCTVSGGSISSSYYYWGWIRQPPGK | 730 |
| | | GLEWIGSIYYSGSAYYNPSLKSRVTISVDTSKNQFSLRLSSVTAAD | |
| | | TAVYYCARHWQEVVPDAFDIWGQGTMVTVSS | |
| | VL | ETTLTQSPAFMSATPGDKVIISCKASQDIDDAMNVVYQQKPGEAPL | 731 |
| | | FIIQSATSPVPGIPPRFSGSGFGTDFSLTINNIESEDAAYYFCLQHD | |
| | | NFPLTFGQGTKLEIK | |
| | scFv | QVQLQESGPGLVKPSETLSLTCTVSGGSISSSYYYWGWIRQPPGK | 732 |
| | | GLEWIGSIYYSGSAYYNPSLKSRVTISVDTSKNQFSLRLSSVTAAD | |
| | | TAVYYCARHWQEWPDAFDIWGQGTMVTVSSGGGGSGGGGSGG | |
| | | GGSETTLTQSPAFMSATPGDKVIISCKASQDIDDAMNVVYQQKPGE | |
| | | APLFIIQSATSPVPGIPPRFSGSGFGTDFSLTINNIESEDAAYYFCLQ | |
| | | HDNFPLTFGQGTKLEIK | |
| BCMA-17 | VH | QVNLRESGPALVKPTQTLTLTCTFSGFSLRTSGMCVSWIRQPPGK | |
| | | ALEVVLARIDVVDEDKFYSTSLKTRLTISKDTSDNQVVLRMTNMDPA | |
| | | DTATYYCARSGAGGTSATAFDIWGPGTMVTVSS | 733 |
| | VL | DIQMTQSPSSLSASVGDRVTITCRASQDIYNNLAVVFQLKPGSAPR | |
| | | SLMYAANKSQSGVPSRFSGSASGTDFTLTISSLQPEDFATYYCQH | |
| | | YYRFPYSFGQGTKLEIK | 734 |
| | scFv | VNLRESGPALVKPTQTLTLTCTFSGFSLRTSGMCVSWIRQPPGKA | |
| | | LEWLARIDWDEDKFYSTSLKTRLTISKDTSDNQVVLRMTNMDPAD | |
| | | TATYYCARSGAGGTSATAFDIWGPGTMVTVSSGGGGSGGGGSG | |
| | | GGGSDIQMTQSPSSLSASVGDRVTITCRASQDIYNNLAVVFQLKPG | |
| | | SAPRSLMYAANKSQSGVPSRFSGSASGTDFTLTISSLQPEDFATY | |
| | | YCQHYYRFPYSFGQGTKLEIK | 735 |
| BCMA-18 | VH | EVQLVESGGGLVKPGGSLRLSCAASGFTFSSYSMNVVVRQAPGK | |
| | | GLEVVVSSISSSSSYIYYADSVKGRFTISRDNAKNSLYLQMNSLRAE | |
| | | DTAVYYCAKTIAAVYAFDIWGQGTTVTVSS | 736 |
| | VL | EIVLTQSPLSLPVTPEEPASISCRSSQSLLHSNGYNYLDVVYLQKPG | |
| | | QSPQLLIYLGSNRASGVPDRFSGSGSGTDFTLKISRVEAEDVGVY | |
| | | YCMQALQTPYTFGQGTKLEIK | 737 |
| | scFv | EVQLVESGGGLVKPGGSLRLSCAASGFTFSSYSMNVVVRQAPGK | |
| | | GLEVVVSSISSSSSYIYYADSVKGRFTISRDNAKNSLYLQMNSLRAE | |
| | | DTAVYYCAKTIAAVYAFDIWGQGTTVTVSSGGGGSGGGGSGGGG | |
| | | SEIVLTQSPLSLPVTPEEPASISCRSSQSLLHSNGYNYLDVVYLQKP | |
| | | GQSPQLLIYLGSNRASGVPDRFSGSGSGTDFTLKISRVEAEDVGV | |
| | | YYCMQALQTPYTFGQGTKLEIK | 738 |
| BCMA-19 | VH | EVQLVESGGGLVKPGGSLRLSCAASGFTFSDYYMSWIRQAPGKG | |
| | | LEVVVSYISSSGSTIYYADSVKGRFTISRDNAKNSLYLQMNSLRAED | |
| | | TAVYYCARDLRGAFDIWGQGTMVTVSS | 739 |
| | VL | SYVLTQSPSVSAAPGYTATISCGGNNIGTKSVHVVYQQKPGQAPLL | |
| | | VIRDDSVRPSKIPGRFSGSNSGNMATLTISGVQAGDEADFYCQVW | |
| | | DSDSEHVVFGGGTKLTVL | 740 |
| | scFv | EVQLVESGGGLVKPGGSLRLSCAASGFTFSDYYMSWIRQAPGKG | |
| | | LEVVVSYISSSGSTIYYADSVKGRFTISRDNAKNSLYLQMNSLRAED | |
| | | TAVYYCARDLRGAFDIWGQGTMVTVSSGGGGSGGGGSGGGGSS | |
| | | YVLTQSPSVSAAPGYTATISCGGNNIGTKSVHVVYQQKPGQAPLLVI | |
| | | RDDSVRPSKIPGRFSGSNSGNMATLTISGVQAGDEADFYCQVVVD | |
| | | SDSEHVVFGGGTKLTVL | 741 |
| BCMA-20 | VH | QVQLVQSGAEVKKPGASVKVSCKPSGYTVTSHYIHVVVRRAPGQG | |
| | | LEWMGMINPSGGVTAYSQTLQGRVTMTSDTSSSTVYMELSSLRS | |
| | | EDTAMYYCAREGSGSGVVYFDFWGRGTLVTVSS | 742 |
| | VL | SYVLTQPPSVSVSPGQTASITCSGDGLSKKYVSVVYQQKAGQSPV | |
| | | VLISRDKERPSGIPDRFSGSNSADTATLTISGTQAMDEADYYCQA | |
| | | WDDTTVVFGGGTKLTVL | 743 |
| | scFv | QVQLVQSGAEVKKPGASVKVSCKPSGYTVTSHYIHVVVRRAPGQG | |
| | | LEWMGMINPSGGVTAYSQTLQGRVTMTSDTSSSTVYMELSSLRS | |
| | | EDTAMYYCAREGSGSGVVYFDFWGRGTLVTVSSGGGGSGGGGS | |
| | | GGGGSSYVLTQPPSVSVSPGQTASITCSGDGLSKKYVSVVYQQKA | |
| | | GQSPVVLISRDKERPSGIPDRFSGSNSADTATLTISGTQAMDEADY | |
| | | YCQAWDDTTVVFGGGTKLTVL | 744 |
| BCMA-21 | VH | QVQLQESGPGLVKPSQTLSLTCTVSGGSISSGGYYWSWIRQHPG | |
| | | KGLEWIGYIYYSGSTYYNPSLKSRVTISVDTSKNQFSLKLSSVTAAD | |
| | | TAVYYCARAGIAARLRGAFDIWGQGTMVTVSS | 745 |
| | VL | DIVMTQSPSSVSASVGDRVIITCRASQGIRNVVLAVVYQQKPGKAPN | |
| | | LLIYAASNLQSGVPSRFSGSGSGADFTLTISSLQPEDVATYYCQKY | |
| | | NSAPFTFGPGTKVDIK | 746 |
| | scFv | QVQLQESGPGLVKPSQTLSLTCTVSGGSISSGGYYWSWIRQHPG | |
| | | KGLEWIGYIYYSGSTYYNPSLKSRVTISVDTSKNQFSLKLSSVTAAD | |
| | | TAVYYCARAGIAARLRGAFDIWGQGTMVTVSSGGGGSGGGGSG | |
| | | GGGSDIVMTQSPSSVSASVGDRVIITCRASQGIRNVVLAVVYQQKPG | |
| | | KAPNLLIYAASNLQSGVPSRFSGSGSGADFTLTISSLQPEDVATYY | |
| | | CQKYNSAPFTFGPGTKVDIK | 747 |
| BCMA-22 | VH | QVQLVQSGAEVKKPGSSVKVSCKASGGTFSSYAISVVVRQAPGQG | |
| | | LEWMGGIIPIFGTANYAQKFQGRVTITADESTSTAYMELSSLRSED | |
| | | TAVYYCARRGGYQLLRWDVGLLRSAFDIWGQGTMVTVSS | 748 |
| | VL | SYVLTQPPSVSVAPGQTARITCGGNNIGSKSVHVVYQQKPGQAPV | |
| | | LVLYGKNNRPSGVPDRFSGSRSGTTASLTITGAQAEDEADYYCSS | |
| | | RDSSGDHLRVFGTGTKVTVL | 749 |
| | scFv | QVQLVQSGAEVKKPGSSVKVSCKASGGTFSSYAISVVVRQAPGQG | 750 |
| | | LEWMGGIIPIFGTANYAQKFQGRVTITADESTSTAYMELSSLRSED | |
| | | TAVYYCARRGGYQLLRWDVGLLRSAFDIWGQGTMVTVSSGGGG | |
| | | SGGGGSGGGGSSYVLTQPPSVSVAPGQTARITCGGNNIGSKSVH | |
| | | VVYQQKPGQAPVLVLYGKNNRPSGVPDRFSGSRSGTTASLTITGA | |
| | | QAEDEADYYCSSRDSSGDHLRVFGTGTKVTVL | |
| BCMA-23 | VH | EVQLQQSGPGLVKPSQTLSLTCAISGDSVSSNSAAVVNWIRQSPSR | |
| | | GLEVVLGRTYYRSKVVYSFYAISLKSRIIINPDTSKNQFSLQLKSVTPE | |
| | | DTAVYYCARSSPEGLFLYVVFDPWGQGTLVTVSS | 751 |
| | VL | SSELTQDPAVSVALGQTIRITCQGDSLGNYYATVVYQQKPGQAPVL | |
| | | VIYGTNNRPSGIPDRFSASSSGNTASLTITGAQAEDEADYYCNSRD | |
| | | SSGHHLLFGTGTKVTVL | 752 |
| | ScFv | EVQLQQSGPGLVKPSQTLSLTCAISGDSVSSNSAAVVNWIRQSPSR | |
| | | GLEVVLGRTYYRSKVVYSFYAISLKSRIIINPDTSKNQFSLQLKSVTPE | |
| | | DTAVYYCARSSPEGLFLYWFDPWGQGTLVTVSSGGDGSGGGGS | |
| | | GGGGSSSELTQDPAVSVALGQTIRITCQGDSLGNYYATVVYQQKP | |
| | | GQAPVLVIYGTNNRPSGIPDRFSASSSGNTASLTITGAQAEDEADY | |
| | | YCNSRDSSGHHLLFGTGTKVTVL | 753 |
| BCMA-24 | VH | EVQLVESGGGLVQPGGSLRLSCAASGFTFSSYAMSVVVRQAPGK | |
| | | GLEVVVSAISGSGGSTYYADSVKGRFTISRDNSKNTLYLQMNSLRA | |
| | | EDTAVYYCAKVEGSGSLDYWGQGTLVTVSS | 754 |
| | VL | EIVMTQSPGTLSLSPGERATLSCRASQSVSSAYLAVVYQQKPGQP | |
| | | PRLLISGASTRATGIPDRFGGSGSGTDFTLTISRLEPEDFAVYYCQ | |
| | | HYGSSFNGSSLFTFGQGTRLEIK | 755 |
| | scFv | EVQLVESGGGLVQPGGSLRLSCAASGFTFSSYAMSVVVRQAPGK | |
| | | GLEVVVSAISGSGGSTYYADSVKGRFTISRDNSKNTLYLQMNSLRA | |
| | | EDTAVYYCAKVEGSGSLDYWGQGTLVTVSSGGGGSGGGGSGG | |
| | | GGSEIVMTQSPGTLSLSPGERATLSCRASQSVSSAYLAVVYQQKP | |
| | | GQPPRLLISGASTRATGIPDRFGGSGSGTDFTLTISRLEPEDFAVY | |
| | | YCQHYGSSFNGSSLFTFGQGTRLEIK | 756 |
| | VH | EVQLVETGGGLVQPGGSLRLSCAASGITFSRYPMSVVVRQAPGKG | |
| | | LEVVVSGISDSGVSTYYADSAKGRFTISRDNSKNTLFLQMSSLRDE | |
| | | DTAVYYCVTRAGSEASDIWGQGTMVTVSS | 757 |
| BCMA-25 | VL | EIVLTQSPATLSLSPGERATLSCRASQSVSNSLAVVYQQKPGQAPR | |
| | | LLIYDASSRATGIPDRFSGSGSGTDFTLTISRLEPEDFAIYYCQQFG | 758 |
| | | TSSGLTFGGGTKLEIK | |
| | scFv | EVQLVETGGGLVQPGGSLRLSCAASGITFSRYPMSVVVRQAPGKG | |
| | | LEVVVSGISDSGVSTYYADSAKGRFTISRDNSKNTLFLQMSSLRDE | |
| | | DTAVYYCVTRAGSEASDIWGQGTMVTVSSGGGGSGGGGSGGG | |
| | | GSEIVLTQSPATLSLSPGERATLSCRASQSVSNSLAVVYQQKPGQA | |
| | | PRLLIYDASSRATGIPDRFSGSGSGTDFTLTISRLEPEDFAIYYCQQ | |
| | | FGTSSGLTFGGGTKLEIK | 759 |
| BCMA-26 | VH | QVQLVESGGGLVQPGGSLRLSCAASGFTFSSYAMSVVVRQAPGK | |
| | | GLEVVVSAISGSGGSTYYADSVKGRFTISRDNAKNSLYLQMNSLRA | |
| | | EDTAIYYCARATYKRELRYYYGMDVWGQGTMVTVSS | 760 |
| | VL | EIVMTQSPGTVSLSPGERATLSCRASQSVSSSFLAVVYQQKPGQA | |
| | | PRLLIYGASSRATGIPDRFSGSGSGTDFTLTISRLEPEDSAVYYCQ | |
| | | QYHSSPSVVTFGQGTRLEIK | 761 |
| | scFv | QVQLVESGGGLVQPGGSLRLSCAASGFTFSSYAMSVVVRQAPGK | |
| | | GLEVVVSAISGSGGSTYYADSVKGRFTISRDNAKNSLYLQMNSLRA | |
| | | EDTAIYYCARATYKRELRYYYGMDVWGQGTMVTVSSGGGGSGG | |
| | | GGSGGGGSEIVMTQSPGTVSLSPGERATLSCRASQSVSSSFLAW | 762 |
| | | YQQKPGQAPRLLIYGASSRATGIPDRFSGSGSGTDFTLTISRLEPE | |
| | | DSAVYYCQQYHSSPSVVTFGQGTRLEIK | |
| BCMA-27 | VH | EVQLVETGGGLVQPGGSLRLSCAASGFTFSSYAMSVVVRQAPGK | |
| | | GLEVVVSAISGSGGSTYYADSVKGRFTISRDNSKNTLYLQMNTLKA | |
| | | EDTAVYYCARATYKRELRYYYGMDVWGQGTTVTVSS | 763 |
| | VL | EIVLTQSPSTLSLSPGESATLSCRASQSVSTTFLAVVYQQKPGQAP | |
| | | RLLIYGSSNRATGIPDRFSGSGSGTDFTLTIRRLEPEDFAVYYCQQ | |
| | | YHSSPSVVTFGQGTKVEIK | 764 |
| | scFv | EVQLVETGGGLVQPGGSLRLSCAASGFTFSSYAMSVVVRQAPGK | |
| | | GLEVVVSAISGSGGSTYYADSVKGRFTISRDNSKNTLYLQMNTLKA | |
| | | EDTAVYYCARATYKRELRYYYGMDVWGQGTTVTVSSGGGGSGG | |
| | | GGSGGGGSEIVLTQSPSTLSLSPGESATLSCRASQSVSTTFLAVVY | |
| | | QQKPGQAPRLLIYGSSNRATGIPDRFSGSGSGTDFTLTIRRLEPED | |
| | | FAVYYCQQYHSSPSVVTFGQGTKVEIK | 765 |
| BCMA-28 | VH | EVQLVETGGGLVQPGRSLRLSCAASGFTFDDYAMHVVVRQAPGK | |
| | | GLEVVVSGISWNSGSIGYADSVKGRFTISRDNAKNSLYLQMNSLRD | |
| | | EDTAVYYCARVGKAVPDVWGQGTTVTVSS | 766 |
| | VL | DIVMTQTPSSLSASVGDRVTITCRASQSISSYLNVVYQQKPGKAPKL | |
| | | LIYAASSLQSGVPSRFSGSGSGTDFTLTISSLQPEDFATYYCQQSY | |
| | | STPYSFGQGTRLEIK | 767 |
| | scFv | EVQLVETGGGLVQPGRSLRLSCAASGFTFDDYAMHVVVRQAPGK | |
| | | GLEVVVSGISWNSGSIGYADSVKGRFTISRDNAKNSLYLQMNSLRD | |
| | | EDTAVYYCARVGKAVPDVWGQGTTVTVSSGGGGSGGGGSGGG | |
| | | GSDIVMTQTPSSLSASVGDRVTITCRASQSISSYLNVVYQQKPGKA | |
| | | PKLLIYAASSLQSGVPSRFSGSGSGTDFTLTISSLQPEDFATYYCQ | |
| | | QSYSTPYSFGQGTRLEIK | 768 |
| BCMA-29 | VH | EVQLVESGGGLVQPGRSLRLSCTASGFTFDDYAMHVVVRQRPGK | |
| | | GLEVVVASINWKGNSLAYGDSVKGRFAISRDNAKNTVFLQMNSLRT | |
| | | EDTAVYYCASHQGVAYYNYAMDVWGRGTLVTVSS | 769 |
| | VL | EIVLTQSPGTLSLSPGERATLSCRATQSIGSSFLAVVYQQRPGQAP | |
| | | RLLIYGASQRATGIPDRFSGRGSGTDFTLTISRVEPEDSAVYYCQH | |
| | | YESSPSVVTFGQGTKVEIK | 770 |
| | scFv | EVQLVESGGGLVQPGRSLRLSCTASGFTFDDYAMHVVVRQRPGK | |
| | | GLEVVVASINWKGNSLAYGDSVKGRFAISRDNAKNTVFLQMNSLRT | |
| | | EDTAVYYCASHQGVAYYNYAMDVWGRGTLVTVSSGGGGSGGG | |
| | | GSGGGGSEIVLTQSPGTLSLSPGERATLSCRATQSIGSSFLAVVYQ | |
| | | QRPGQAPRLLIYGASQRATGIPDRFSGRGSGTDFTLTISRVEPEDS | |
| | | AVYYCQHYESSPSVVTFGQGTKVEIK | 771 |
| BCMA-30 | VH | EVQLVESGGGLVQPGGSLRLSCAASGFTFSSYAMSVVVRQAPGK | |
| | | GLEVVVSAISGSGGSTYYADSVKGRFTISRDNSKNTLYLQMNSLRA | |
| | | EDTAVYYCAKVVRDGMDVWGQGTTVTVSS | 772 |
| | VL | EIVLTQSPATLSLSPGERATLSCRASQSVSSSYLAVVYQQKPGQAP | |
| | | RLLIYGASSRATGIPDRFSGNGSGTDFTLTISRLEPEDFAVYYCQQ | |
| | | YGSPPRFTFGPGTKVDIK | 773 |
| | scFv | EVQLVESGGGLVQPGGSLRLSCAASGFTFSSYAMSVVVRQAPGK | |
| | | GLEVVVSAISGSGGSTYYADSVKGRFTISRDNSKNTLYLQMNSLRA | |
| | | EDTAVYYCAKVVRDGMDVWGQGTTVTVSSGGGGSGGGGSGGG | |
| | | GSEIVLTQSPATLSLSPGERATLSCRASQSVSSSYLAVVYQQKPGQ | |
| | | APRLLIYGASSRATGIPDRFSGNGSGTDFTLTISRLEPEDFAVYYC | |
| | | QQYGSPPRFTFGPGTKVDIK | 774 |
| BCMA-31 | VH | EVQLLESGGGLVQPGGSLRLSCAASGFTFSSYAMSVVVRQAPGKG | |
| | | LEVVVSAISGSGGSTYYADSVKGRFTISRDNSKNTLYLQMNSLRAE | 775 |
| | | DTAVYYCAKIPQTGTFDYWGQGTLVTVSS | |
| | VL | EIVLTQSPGTLSLSPGERATLSCRASQSVSSSYLAVVYQQRPGQAP | |
| | | RLLIYGASSRATGIPDRFSGSGSGTDFTLTISRLEPEDFAVYYCQH | |
| | | YGSSPSVVTFGQGTRLEIK | 776 |
| | scFv | EVQLLESGGGLVQPGGSLRLSCAASGFTFSSYAMSVVVRQAPGKG | |
| | | LEVVVSAISGSGGSTYYADSVKGRFTISRDNSKNTLYLQMNSLRAE | |
| | | DTAVYYCAKIPQTGTFDYWGQGTLVTVSSGGGGSGGGGSGGGG | |
| | | SEIVLTQSPGTLSLSPGERATLSCRASQSVSSSYLAVVYQQRPGQA | |
| | | PRLLIYGASSRATGIPDRFSGSGSGTDFTLTISRLEPEDFAVYYCQ | |
| | | HYGSSPSVVTFGQGTRLEIK | 777 |
| BCMA-32 | VH | EVQLVETGGGLVQPGGSLRLSCAASGFTFSSYAMSVVVRQAPGK | |
| | | GLEVVVSAISGSGGSTYYADSVKGRFTMSRENDKNSVFLQMNSLR | |
| | | VEDTGVYYCARANYKRELRYYYGMDVWGQGTMVTVSS | 778 |
| | VL | EIVMTQSPGTLSLSPGESATLSCRASQRVASNYLAVVYQHKPGQA | |
| | | PSLLISGASSRATGVPDRFSGSGSGTDFTLAISRLEPEDSAVYYCQ | |
| | | HYDSSPSVVTFGQGTKVEIK | 779 |
| | scFv | EVQLVETGGGLVQPGGSLRLSCAASGFTFSSYAMSVVVRQAPGK | |
| | | GLEVVVSAISGSGGSTYYADSVKGRFTMSRENDKNSVFLQMNSLR | |
| | | VEDTGVYYCARANYKRELRYYYGMDVWGQGTMVTVSSGGGGS | |
| | | GGGGSGGGGSEIVMTQSPGTLSLSPGESATLSCRASQRVASNYL | |
| | | AVVYQHKPGQAPSLLISGASSRATGVPDRFSGSGSGTDFTLAISRL | |
| | | EPEDSAVYYCQHYDSSPSVVTFGQGTKVEIK | 780 |
| BCMA-33 | VH | EVQLLETGGGLVQPGGSLRLSCAASGFSFSSYAMSVVVRQAPGKG | |
| | | LEVVVSAISGSGGSTYYADSVKGRFTISRDNSKNTLYLQMNSLRAE | |
| | | DTAVYYCAKALVGATGAFDIWGQGTLVTVSS | 781 |
| | VL | EIVLTQSPGTLSLSPGERATLSCRASQSLSSNFLAVVYQQKPGQAP | |
| | | GLLIYGASNWATGTPDRFSGSGSGTDFTLTITRLEPEDFAVYYCQY | |
| | | YGTSPMYTFGQGTKVEIK | 782 |
| | scFv | EVQLLETGGGLVQPGGSLRLSCAASGFSFSSYAMSVVVRQAPGKG | |
| | | LEVVVSAISGSGGSTYYADSVKGRFTISRDNSKNTLYLQMNSLRAE | |
| | | DTAVYYCAKALVGATGAFDIWGQGTLVTVSSGGGGSGGGGSGG | |
| | | GGSEIVLTQSPGTLSLSPGERATLSCRASQSLSSNFLAVVYQQKPG | |
| | | QAPGLLIYGASNWATGTPDRFSGSGSGTDFTLTITRLEPEDFAVYY | |
| | | CQYYGTSPMYTFGQGTKVEIK | 783 |
| BCMA-34 | VH | EVQLLESGGGLVQPGGSLRLSCAASGFTFSSYAMSVVVRQAPGKG | |
| | | LEVVVSAISGSGGSTYYADSVKGRFTISRDNSKNTLYLQMNSLRAE | |
| | | DTAVYYCVLVVFGEGFDPWGQGTLVTVSS | 784 |
| | VL | DIVLTQSPLSLPVTPGEPASISCRSSQSLLHSNGYNYLDVVYLQKPG | |
| | | QSPQLLIYLGSNRASGVPDRFSGSGSGTDFTLKISRVEAEDVGVY | |
| | | YCMQALQTPLTFGGGTKVDIK | 785 |
| | scFv | EVQLLESGGGLVQPGGSLRLSCAASGFTFSSYAMSVVVRQAPGKG | |
| | | LEVVVSAISGSGGSTYYADSVKGRFTISRDNSKNTLYLQMNSLRAE | |
| | | DTAVYYCVLVVFGEGFDPWGQGTLVTVSSGGGGSGGGGSGGGG | |
| | | SDIVLTQSPLSLPVTPGEPASISCRSSQSLLHSNGYNYLDVVYLQKP | |
| | | GQSPQLLIYLGSNRASGVPDRFSGSGSGTDFTLKISRVEAEDVGV | |
| | | YYCMQALQTPLTFGGGTKVDIK | 786 |
| BCMA-35 | VH | QVQLVESGGGLVQPGGSLRLSCAASGFTFSSYAMSVVVRQAPGK | |
| | | GLEVVVSAISGSGGSTYYADSVKGRFTISRDNSKNTLYLQMNSLRA | |
| | | EDTAVYYCAKVGYDSSGYYRDYYGMDVWGQGTTVTVSS | 787 |
| | VL | EIVLTQSPGTLSLSPGERATLSCRASQSVSSSYLAVVYQQKPGQAP | |
| | | RLLIYGTSSRATGISDRFSGSGSGTDFTLTISRLEPEDFAVYYCQHY | |
| | | GNSPPKFTFGPGTKLEIK | 788 |
| | scFv | QVQLVESGGGLVQPGGSLRLSCAASGFTFSSYAMSVVVRQAPGK | |
| | | GLEVVVSAISGSGGSTYYADSVKGRFTISRDNSKNTLYLQMNSLRA | |
| | | EDTAVYYCAKVGYDSSGYYRDYYGMDVWGQGTTVTVSSGGGGS | |
| | | GGGGSGGGGSEIVLTQSPGTLSLSPGERATLSCRASQSVSSSYLA | |
| | | VVYQQKPGQAPRLLIYGTSSRATGISDRFSGSGSGTDFTLTISRLEP | |
| | | EDFAVYYCQHYGNSPPKFTFGPGTKLEIK | 789 |
| BCMA-36 | VH | EVQLVESGGGLVQPGGSLRLSCAASGFTFSSYAMSVVVRQAPGK | |
| | | GLEVVVSAISGSGGSTYYADSVKGRFTISRDNSKNTLYLQMNSLRA | |
| | | EDTAVYYCAKMGWSSGYLGAFDIWGQGTTVTVSS | 790 |
| | VL | EIVLTQSPGTLSLSPGERATLSCRASQSVASSFLAVVYQQKPGQAP | |
| | | RLLIYGASGRATGIPDRFSGSGSGTDFTLTISRLEPEDFAVYYCQH | |
| | | YGGSPRLTFGGGTKVDIK | 791 |
| | scFv | EVQLVESGGGLVQPGGSLRLSCAASGFTFSSYAMSVVVRQAPGK | |
| | | GLEVVVSAISGSGGSTYYADSVKGRFTISRDNSKNTLYLQMNSLRA | |
| | | EDTAVYYCAKMGWSSGYLGAFDIWGQGTTVTVSSGGGGSGGGG | |
| | | SGGGGSEIVLTQSPGTLSLSPGERATLSCRASQSVASSFLAVVYQQ | |
| | | KPGQAPRLLIYGASGRATGIPDRFSGSGSGTDFTLTISRLEPEDFA | |
| | | VYYCQHYGGSPRLTFGGGTKVDIK | 792 |
| BCMA-37 | VH | QIQLVQSGPDLKKPGETVKLSCKASGYTFTNFGMNVVVKQAPGKG | |
| | | FKWMAWINTYTGESYFADDFKGRFAFSVETSATTAYLQINNLKTE | |
| | | DTATYFCARGEIYYGYDGGFAYWGQGTLVTVSA | 793 |
| | VL | DVVMTQSHRFMSTSVGDRVSITCRASQDVNTAVSVVYQQKPGQS | |
| | | PKLLIFSASYRYTGVPDRFTGSGSGADFTLTISSVQAEDLAVYYCQ | |
| | | QHYSTPVVTFGGGTKLDIK | 794 |
| | scFv | QIQLVQSGPDLKKPGETVKLSCKASGYTFTNFGMNVVVKQAPGKG | |
| | | FKWMAWINTYTGESYFADDFKGRFAFSVETSATTAYLQINNLKTE | |
| | | DTATYFCARGEIYYGYDGGFAYWGQGTLVTVSAGGGGSGGGGS | |
| | | GGGGSDVVMTQSHRFMSTSVGDRVSITCRASQDVNTAVSVVYQQ | |
| | | KPGQSPKLLIFSASYRYTGVPDRFTGSGSGADFTLTISSVQAEDLA | |
| | | VYYCQQHYSTPVVTFGGGTKLDIK | 795 |
| BCMA-38 | VH | QIQLVQSGPELKKPGETVKISCKASGYTFTDYSINVVVKRAPGKGLK | |
| | | WMGWINTETREPAYAYDFRGRFAFSLETSASTAYLQINNLKYEDT | |
| | | ATYFCALDYSYAMDYWGQGTSVTVSS | 796 |
| | VL | DIVLTQSPASLAMSLGKRATISCRASESVSVIGAHLIHVVYQQKPGQ | |
| | | PPKLLIYLASNLETGVPARFSGSGSGTDFTLTIDPVEEDDVAIYSCL | |
| | | QSRIFPRTFGGGTKLEIK | 797 |
| | scFv | QIQLVQSGPELKKPGETVKISCKASGYTFTDYSINVVVKRAPGKGLK | |
| | | WMGWINTETREPAYAYDFRGRFAFSLETSASTAYLQINNLKYEDT | |
| | | ATYFCALDYSYAMDYWGQGTSVTVSSGGGGSGGGGSGGGGSQI | |
| | | QLVQSGPELKKPGETVKISCKASGYTFTDYSINVVVKRAPGKGLKW | |
| | | MGWINTETREPAYAYDFRGRFAFSLETSASTAYLQINNLKYEDTAT | |
| | | YFCALDYSYAMDYWGQGTSVTVSS | 798 |
| BCMA-39 | VH | QIQLVQSGPELKKPGETVKISCKASGYTFRHYSMNVVVKQAPGKGL | |
| | | KVVMGRINTESGVPIYADDFKGRFAFSVETSASTAYLVINNLKDEDT | |
| | | ASYFCSNDYLYSLDFWGQGTALTVSS | 799 |
| | VL | DIVLTQSPPSLAMSLGKRATISCRASESVTILGSHLIYVVYQQKPGQ | |
| | | PPTLLIQLASNVQTGVPARFSGSGSRTDFTLTIDPVEEDDVAVYYC | |
| | | LQSRTIPRTFGGGTKLEIK | 800 |
| | scFv | QIQLVQSGPELKKPGETVKISCKASGYTFRHYSMNVVVKQAPGKGL | |
| | | KVVMGRINTESGVPIYADDFKGRFAFSVETSASTAYLVINNLKDEDT | |
| | | ASYFCSNDYLYSLDFWGQGTALTVSSGGGGSGGGGSGGGGSDI | |
| | | VLTQSPPSLAMSLGKRATISCRASESVTILGSHLIYWYQQKPGQPP | |
| | | TLLIQLASNVQTGVPARFSGSGSRTDFTLTIDPVEEDDVAVYYCLQ | |
| | | SRTIPRTFGGGTKLEIK | 801 |
| BCMA-40 | VH | QIQLVQSGPELKKPGETVKISCKASGYTFTHYSMNVVVKQAPGKGL | |
| | | KVVMGRINTETGEPLYADDFKGRFAFSLETSASTAYLVINNLKNEDT | |
| | | ATFFCSNDYLYSCDYWGQGTTLTVSS | 802 |
| | VL | DIVLTQSPPSLAMSLGKRATISCRASESVTILGSHLIYVVYQQKPGQ | |
| | | PPTLLIQLASNVQTGVPARFSGSGSRTDFTLTIDPVEEDDVAVYYC | |
| | | LQSRTIPRTFGGGTKLEIK | 800 |
| | scFv | QIQLVQSGPELKKPGETVKISCKASGYTFTHYSMNVVVKQAPGKGL | |
| | | KVVMGRINTETGEPLYADDFKGRFAFSLETSASTAYLVINNLKNEDT | |
| | | ATFFCSNDYLYSCDYWGQGTTLTVSSGGGGSGGGGSGGGGSDI | |
| | | VLTQSPPSLAMSLGKRATISCRASESVTILGSHLIYWYQQKPGQPP | |
| | | TLLIQLASNVQTGVPARFSGSGSRTDFTLTIDPVEEDDVAVYYCLQ | |
| | | SRTIPRTFGGGTKLEIK | 803 |

TABLE 16B
BCMA Binders - Light chain CDR sequences according to Kabat numbering
scheme

| | | SEQ ID | | SEQ ID | | SEQ ID |
|---|---|---|---|---|---|---|
| Antibody | CDR-L1 | NO: | CDR-L2 | NO: | CDR-L3 | NO: |
| BCMA-1 | RASQSISSYLN | 804 | AASSLQS | 836 | QQSYSTPYT | 865 |
| BCMA-2 | RASQSISSSFLA | 805 | GASRRAT | 837 | QQYHSSPSVVT | 866 |
| BCMA-3 | RSSQSLLHSNGYNYLD | 806 | LGSNRAS | 838 | MQALQTPYT | 867 |
| BCMA-4 | KSSQSLLRNDGKTPLY | 807 | EVSNRFS | 839 | MQNIQFPS | 868 |
| BCMA-5 | RSSQSLLHSNGYNYLN | 808 | LGSKRAS | 840 | MQALQTPYT | 867 |
| BCMA-6 | RASQSISSYLN | 804 | GASTLAS | 841 | QQSYKRAS | 869 |
| BCMA-7 | RSSQSLLYSNGYNYVD | 809 | LGSNRAS | 838 | MQGRQFPYS | 870 |
| BCMA-8 | RASQSVSSNLA | 810 | GASTRAS | 842 | QQYGSSLT | 871 |
| BCMA-9 | RASQSVSSKLA | 811 | GASIRAT | 843 | QQYGSSSVVT | 872 |
| BCMA-10 | RASQSVGSTNLA | 812 | DASNRAT | 684 | QQYGSSPPVVT | 873 |
| BCMA-11 | RASQSISSYLN | 804 | AASSLQS | 836 | QQSYTLA | 874 |
| BCMA-12 | KSSESLVHNSGKTYLN | 813 | EVSNRDS | 844 | MQGTHWPGT | 875 |
| BCMA-13 | QASEDINKFLN | 814 | DASTLQT | 845 | QQYESLPLT | 876 |
| BCMA-14 | RASQSVGSNLA | 815 | GASTRAT | 846 | QQYNDVVLPVT | 877 |
| BCMA-15 | RASQSIGSSSLA | 816 | GASSRAS | 847 | QQYAGSPPFT | 878 |
| BCMA-16 | KASQDIDDAMN | 817 | SATSPVP | 848 | LQHDNFPLT | 879 |
| BCMA-17 | RASQDIYNNLA | 818 | AANKSQS | 849 | QHYYRFPYS | 880 |
| BCMA-18 | RSSQSLLHSNGYNYLD | 806 | LGSNRAS | 838 | MQALQTPYT | 867 |
| BCMA-19 | GGNNIGTKSVH | 819 | DDSVRPS | 850 | QVVVDSDSEHVV | 881 |
| BCMA-20 | SGDGLSKKYVS | 820 | RDKERPS | 851 | QAVVDDTTVV | 882 |
| BCMA-21 | RASQGIRNVVLA | 821 | AASNLQS | 852 | QKYNSAPFT | 883 |
| BCMA-22 | GGNNIGSKSVH | 822 | GKNNRPS | 853 | SSRDSSGDHLRV | 884 |
| BCMA-23 | QGDSLGNYYAT | 823 | GTNNRPS | 854 | NSRDSSGHHLL | 885 |
| BCMA-24 | RASQSVSSAYLA | 824 | GASTRAT | 846 | QHYGSSFNGSSLFT | 886 |
| BCMA-25 | RASQSVSNSLA | 825 | DASSRAT | 855 | QQFGTSSGLT | 887 |
| BCMA-26 | RASQSVSSSFLA | 826 | GASSRAT | 856 | QQYHSSPSVVT | 866 |
| BCMA-27 | RASQSVSTTFLA | 827 | GSSNRAT | 857 | QQYHSSPSVVT | 866 |
| BCMA-28 | RASQSISSYLN | 804 | AASSLQS | 836 | QQSYSTPYS | 888 |
| BCMA-29 | RATQSIGSSFLA | 828 | GASQRAT | 858 | QHYESSPSVVT | 889 |
| BCMA-30 | RASQSVSSSYLA | 829 | GASSRAT | 856 | QQYGSPPRFT | 890 |
| BCMA-31 | RASQSVSSSYLA | 829 | GASSRAT | 856 | QHYGSSPSVVT | 891 |
| BCMA-32 | RASQRVASNYLA | 830 | GASSRAT | 856 | QHYDSSPSVVT | 892 |
| BCMA-33 | RASQSLSSNFLA | 831 | GASNWAT | 859 | QYYGTSPMYT | 893 |
| BCMA-34 | RSSQSLLHSNGYNYLD | 806 | LGSNRAS | 838 | MQALQTPLT | 894 |
| BCMA-35 | RASQSVSSSYLA | 829 | GTSSRAT | 860 | QHYGNSPPKFT | 895 |
| BCMA-36 | RASQSVASSFLA | 832 | GASGRAT | 861 | QHYGGSPRLT | 896 |
| BCMA-37 | RASQDVNTAVS | 833 | SASYRYT | 862 | QQHYSTPVVT | 897 |
| BCMA-38 | RASESVSVIGAHLIH | 834 | LASNLET | 863 | LQSRIFPRT | 898 |
| BCMA-39 | RASESVTILGSHLIY | 835 | LASNVQT | 864 | LQSRTIPRT | 899 |
| BCMA-40 | RASESVTILGSHLIY | 835 | LASNVQT | 864 | LQSRTIPRT | 899 |

TABLE 16C
BCMA Binders - Light chain CDR sequences according to Chothia
numbering scheme

| | | SEQ ID | | SEQ ID | | SEQ ID |
|---|---|---|---|---|---|---|
| Antibody | CDR-L1 | NO: | CDR-L2 | NO: | CDR-L3 | NO: |
| BCMA-1 | SQSISSY | 900 | AAS | 931 | SYSTPY | 944 |
| BCMA-2 | SQSISSSF | 901 | GAS | 932 | YHSSPSW | 945 |
| BCMA-3 | SQSLLHSNGYNY | 902 | LGS | 933 | ALQTPY | 946 |
| BCMA-4 | SQSLLRNDGKTP | 903 | EVS | 934 | NIQFP | 947 |
| BCMA-5 | SQSLLHSNGYNY | 902 | LGS | 933 | ALQTPY | 946 |
| BCMA-6 | SQSISSY | 900 | GAS | 932 | SYKRA | 948 |
| BCMA-7 | SQSLLYSNGYNY | 904 | LGS | 933 | GRQFPY | 949 |
| BCMA-8 | SQSVSSN | 905 | GAS | 932 | YGSSL | 950 |
| BCMA-9 | SQSVSSK | 906 | GAS | 932 | YGSSSW | 951 |
| BCMA-10 | SQSVGSTN | 907 | DAS | 686 | YGSSPPW | 952 |
| BCMA-11 | SQSISSY | 900 | AAS | 931 | SYTL | 953 |
| BCMA-12 | SESLVHNSGKTY | 908 | EVS | 934 | GTHVVPG | 954 |
| BCMA-13 | SEDINKF | 909 | DAS | 686 | YESLPL | 955 |
| BCMA-14 | SQSVGSN | 910 | GAS | 932 | YNDVVLPV | 956 |
| BCMA-15 | SQSIGSSS | 911 | GAS | 932 | YAGSPPF | 957 |
| BCMA-16 | SQDIDDA | 912 | SAT | 935 | HDNFPL | 958 |
| BCMA-17 | SQDIYNN | 913 | AAN | 936 | YYRFPY | 959 |
| BCMA-18 | SQSLLHSNGYNY | 902 | LGS | 933 | ALQTPY | 946 |
| BCMA-19 | NNIGTKS | 914 | DDS | 937 | VVDSDSEHV | 960 |
| BCMA-20 | DGLSKKY | 915 | RDK | 938 | VVDDTTV | 961 |
| BCMA-21 | SQGIRNW | 916 | AAS | 931 | YNSAPF | 962 |
| BCMA-22 | NNIGSKS | 917 | GKN | 939 | RDSSGDHLR | 963 |
| BCMA-23 | DSLGNYY | 918 | GTN | 685 | RDSSGHHL | 964 |
| BCMA-24 | SQSVSSAY | 919 | GAS | 932 | YGSSFNGSSLF | 965 |
| BCMA-25 | SQSVSNS | 920 | DAS | 686 | FGTSSGL | 966 |
| BCMA-26 | SQSVSSSF | 921 | GAS | 932 | YHSSPSW | 945 |
| BCMA-27 | SQSVSTTF | 922 | GSS | 940 | YHSSPSW | 945 |
| BCMA-28 | SQSISSY | 900 | AAS | 931 | SYSTPY | 944 |
| BCMA-29 | TQSIGSSF | 923 | GAS | 932 | YESSPSW | 967 |
| BCMA-30 | SQSVSSSY | 924 | GAS | 932 | YGSPPRF | 968 |
| BCMA-31 | SQSVSSSY | 924 | GAS | 932 | YGSSPSW | 969 |
| BCMA-32 | SQRVASNY | 925 | GAS | 932 | YDSSPSW | 970 |
| BCMA-33 | SQSLSSNF | 926 | GAS | 932 | YGTSPMY | 971 |
| BCMA-34 | SQSLLHSNGYNY | 902 | LGS | 933 | ALQTPL | 972 |
| BCMA-35 | SQSVSSSY | 924 | GTS | 941 | YGNSPPKF | 973 |
| BCMA-36 | SQSVASSF | 927 | GAS | 932 | YGGSPRL | 974 |
| BCMA-37 | SQDVNTA | 928 | SAS | 942 | HYSTPW | 975 |
| BCMA-38 | SESVSVIGAHL | 929 | LAS | 943 | SRIFPR | 976 |
| BCMA-39 | SESVTILGSHL | 930 | LAS | 943 | SRTIPR | 977 |
| BCMA-40 | SESVTILGSHL | 930 | LAS | 943 | SRTIPR | 977 |

TABLE 16D
BCMA Binders - Light chain CDR sequences according to combination of
Kabat and Chothia numbering schemes

| | | SEQ ID | | SEQ ID | | SEQ ID |
|---|---|---|---|---|---|---|
| Antibody | CDR-L1 | NO: | CDR-L2 | NO: | CDR-L3 | NO: |
| BCMA-1 | RASQSISSYLN | 804 | AASSLQS | 836 | QQSYSTPYT | 865 |
| BCMA-2 | RASQSISSSFLA | 805 | GASRRAT | 837 | QQYHSSPSVVT | 866 |
| BCMA-3 | RSSQSLLHSNGYN | 806 | LGSNRAS | 838 | MQALQTPYT | 867 |
| | YLD | | | | | |
| BCMA-4 | KSSQSLLRNDGKTP | 807 | EVSNRFS | 839 | MQNIQFPS | 868 |
| | LY | | | | | |
| BCMA-5 | RSSQSLLHSNGYN | 808 | LGSKRAS | 840 | MQALQTPYT | 867 |
| | YLN | | | | | |
| BCMA-6 | RASQSISSYLN | 804 | GASTLAS | 841 | QQSYKRAS | 869 |
| BCMA-7 | RSSQSLLYSNGYN | 809 | LGSNRAS | 838 | MQGRQFPYS | 870 |
| | YVD | | | | | |
| BCMA-8 | RASQSVSSNLA | 810 | GASTRAS | 842 | QQYGSSLT | 871 |
| BCMA-9 | RASQSVSSKLA | 811 | GASIRAT | 843 | QQYGSSSVVT | 872 |
| BCMA-10 | RASQSVGSTNLA | 812 | DASNRAT | 684 | QQYGSSPPVVT | 873 |
| BCMA-11 | RASQSISSYLN | 804 | AASSLQS | 836 | QQSYTLA | 874 |
| BCMA-12 | KSSESLVHNSGKTY | 813 | EVSNRDS | 844 | MQGTHVVPGT | 875 |
| | LN | | | | | |
| BCMA-13 | QASEDINKFLN | 814 | DASTLQT | 845 | QQYESLPLT | 876 |
| BCMA-14 | RASQSVGSNLA | 815 | GASTRAT | 846 | QQYNDVVLPVT | 877 |
| BCMA-15 | RASQSIGSSSLA | 816 | GASSRAS | 847 | QQYAGSPPFT | 878 |
| BCMA-16 | KASQDIDDAMN | 817 | SATSPVP | 848 | LQHDNFPLT | 879 |
| BCMA-17 | RASQDIYNNLA | 818 | AANKSQS | 849 | QHYYRFPYS | 880 |
| BCMA-18 | RSSQSLLHSNGYN | 806 | LGSNRAS | 838 | MQALQTPYT | 867 |
| | YLD | | | | | |
| BCMA-19 | GGNNIGTKSVH | 819 | DDSVRPS | 850 | QVVVDSDSEHVV | 881 |
| BCMA-20 | SGDGLSKKYVS | 820 | RDKERPS | 851 | QAVVDDTTVV | 882 |
| BCMA-21 | RASQGIRNWLA | 821 | AASNLQS | 852 | QKYNSAPFT | 883 |
| BCMA-22 | GGNNIGSKSVH | 822 | GKNNRPS | 853 | SSRDSSGDHLRV | 884 |
| BCMA-23 | QGDSLGNYYAT | 823 | GTNNRPS | 854 | NSRDSSGHHLL | 885 |
| BCMA-24 | RASQSVSSAYLA | 824 | GASTRAT | 846 | QHYGSSFNGSSLFT | 886 |
| BCMA-25 | RASQSVSNSLA | 825 | DASSRAT | 855 | QQFGTSSGLT | 887 |
| BCMA-26 | RASQSVSSSFLA | 826 | GASSRAT | 856 | QQYHSSPSVVT | 866 |
| BCMA-27 | RASQSVSTTFLA | 827 | GSSNRAT | 857 | QQYHSSPSVVT | 866 |
| BCMA-28 | RASQSISSYLN | 804 | AASSLQS | 836 | QQSYSTPYS | 888 |
| BCMA-29 | RATQSIGSSFLA | 828 | GASQRAT | 858 | QHYESSPSVVT | 889 |
| BCMA-30 | RASQSVSSSYLA | 829 | GASSRAT | 856 | QQYGSPPRFT | 890 |
| BCMA-31 | RASQSVSSSYLA | 829 | GASSRAT | 856 | QHYGSSPSVVT | 891 |
| BCMA-32 | RASQRVASNYLA | 830 | GASSRAT | 856 | QHYDSSPSVVT | 892 |
| BCMA-33 | RASQSLSSNFLA | 831 | GASNWAT | 859 | QYYGTSPMYT | 893 |
| BCMA-34 | RSSQSLLHSNGYNYLD | 806 | LGSNRAS | 838 | MQALQTPLT | 894 |
| BCMA-35 | RASQSVSSSYLA | 829 | GTSSRAT | 860 | QHYGNSPPKFT | 895 |
| BCMA-36 | RASQSVASSFLA | 832 | GASGRAT | 861 | QHYGGSPRLT | 896 |
| BCMA-37 | RASQDVNTAVS | 833 | SASYRYT | 862 | QQHYSTPVVT | 897 |
| BCMA-38 | RASESVSVIGAHLIH | 834 | LASNLET | 863 | LQSRIFPRT | 898 |
| BCMA-39 | RASESVTILGSHLIY | 835 | LASNVQT | 864 | LQSRTIPRT | 899 |
| BCMA-40 | RASESVTILGSHLIY | 835 | LASNVQT | 864 | LQSRTIPRT | 899 |

TABLE 16E
BCMA Binders - Heavy chain CDR sequences according to Kabat numbering scheme

| | | SEQ ID | | SEQ ID | | SEQ ID |
|---|---|---|---|---|---|---|
| Antibody | CDR-H1 | NO: | CDR-H2 | NO: | CDR-H3 | NO: |
| BCMA-1 | NHGMS | 978 | GIVYSGSTYYAASVKG | 997 | HGGESDV | 1019 |
| BCMA-2 | NYAMS | 979 | GISRSGENTYYADSVKG | 998 | SPAHYYGGMDV | 1020 |
| BCMA-3 | DYAMH | 980 | GISWNSGSIGYADSVKG | 999 | HSFLAY | 1021 |
| BCMA-4 | NHGMS | 978 | GIVYSGSTYYAASVKG | 997 | HGGESDV | 1019 |
| BCMA-5 | NFGIN | 981 | WINPKNNNTNYAQKFQG | 1000 | GPYYYQSYMDV | 1022 |
| BCMA-6 | SDAMT | 982 | VISGSGGTTYYADSVKG | 1001 | LDSSGYYYARGPRY | 1023 |
| BCMA-7 | NYGIT | 983 | WISAYNGNTNYAQKFQG | 1002 | GPYYYYMDV | 1024 |
| BCMA-8 | NHGMS | 978 | GIVYSGSTYYAASVKG | 997 | HGGESDV | 1019 |
| BCMA-9 | NHGMS | 978 | GIVYSGSTYYAASVKG | 997 | HGGESDV | 1019 |
| BCMA-10 | NHGMS | 978 | GIVYSGSTYYAASVKG | 997 | HGGESDV | 1019 |
| BCMA-11 | DYYMS | 984 | YISSSGSTIYYADSVKG | 1003 | ESGDGMDV | 1025 |
| BCMA-12 | DYYMS | 984 | YISSSGNTIYYADSVKG | 1004 | STMVREDY | 1026 |
| BCMA-13 | NHGMS | 978 | GIVYSGSTYYAASVKG | 997 | HGGESDV | 1019 |
| BCMA-14 | NHGMS | 978 | GIVYSGSTYYAASVKG | 997 | HGGESDV | 1019 |
| BCMA-15 | NHGMS | 978 | GIVYSGSTYYAASVKG | 997 | HGGESDV | 1019 |
| BCMA-16 | SSYYYWG | 985 | SIYYSGSAYYNPSLKS | 1005 | HWQEWPDAFDI | 1027 |
| BCMA-17 | TSGMCVS | 986 | RIDWDEDKFYSTSLKT | 1006 | SGAGGTSATAFDI | 1028 |
| BCMA-18 | SYSMN | 987 | SISSSSSYIYYADSVKG | 1007 | TIAAVYAFDI | 1029 |
| BCMA-19 | DYYMS | 984 | YISSSGSTIYYADSVKG | 1003 | DLRGAFDI | 1030 |
| BCMA-20 | SHYIH | 988 | MINPSGGVTAYSQTLQG | 1008 | EGSGSGWYFDF | 1031 |
| BCMA-21 | SGGYYWS | 989 | YIYYSGSTYYNPSLKS | 1009 | AGIAARLRGAFDI | 1032 |
| BCMA 22 | SYAIS | 990 | GIIPIFGTANYAQKFQG | 1010 | RGGYQLLRWDVGLL | 1033 |
| | | | | | RSAFDI | |
| BCMA-23 | SNSAAWN | 991 | RTYYRSKWYSFYAISLK | 1011 | SSPEGLFLYWFDP | 1034 |
| | | | S | | | |
| BCMA-24 | SYAMS | 992 | AISGSGGSTYYADSVKG | 1012 | VEGSGSLDY | 1035 |
| BCMA-25 | RYPMS | 993 | GISDSGVSTYYADSAKG | 1013 | RAGSEASDI | 1036 |
| BCMA-26 | SYAMS | 992 | AISGSGGSTYYADSVKG | 1012 | ATYKRELRYYYGMD | 1037 |
| | | | | | V | |
| BCMA-27 | SYAMS | 992 | AISGSGGSTYYADSVKG | 1012 | ATYKRELRYYYGMD | 1037 |
| | | | | | V | |
| BCMA-28 | DYAMH | 980 | GISVVNSGSIGYADSVKG | 999 | VGKAVPDV | 1038 |
| BCMA-29 | DYAMH | 980 | SINWKGNSLAYGDSVK | 1014 | HQGVAYYNYAMDV | 1039 |
| | | | G | | | |
| BCMA-30 | SYAMS | 992 | AISGSGGSTYYADSVKG | 1012 | VVRDGMDV | 1040 |
| BCMA-31 | SYAMS | 992 | AISGSGGSTYYADSVKG | 1012 | IPQTGTFDY | 1041 |
| BCMA-32 | SYAMS | 992 | AISGSGGSTYYADSVKG | 1012 | ANYKRELRYYYGMD | 1042 |
| | | | | | V | |
| BCMA-33 | SYAMS | 992 | AISGSGGSTYYADSVKG | 1012 | ALVGATGAFDI | 1043 |
| BCMA-34 | SYAMS | 992 | AISGSGGSTYYADSVKG | 1012 | WFGEGFDP | 1044 |
| BCMA-35 | SYAMS | 992 | AISGSGGSTYYADSVKG | 1012 | VGYDSSGYYRDYYG | 1045 |
| | | | | | MDV | |
| BCMA-36 | SYAMS | 992 | AISGSGGSTYYADSVKG | 1012 | MGWSSGYLGAFDI | 1046 |
| BCMA-37 | NFGMN | 994 | WINTYTGESYFADDFKG | 1015 | GEIYYGYDGGFAY | 1047 |
| BCMA-36 | DYSIN | 995 | WINTETREPAYAYDFRG | 1016 | DYSYAMDY | 1048 |
| BCMA-39 | HYSMN | 996 | RINTESGVPIYADDFKG | 1017 | DYLYSLDF | 1049 |
| BCMA-40 | HYSMN | 996 | RINTETGEPLYADDFKG | 1018 | DYLYSCDY | 1050 |

TABLE 16F
BCMA Binders-Heavy chain CDR sequences according to Chothia numbering
scheme

| | | SEQ ID | | SEQ ID | | SEQ ID |
|---|---|---|---|---|---|---|
| Antibody | CDR-H1 | NO: | CDR-H2 | NO: | CDR-H3 | NO: |
| BCMA-1 | GFALSNH | 1051 | VYSGS | 1071 | HGGESDV | 1019 |
| BCMA-2 | GFTFSNY | 1052 | SRSGEN | 1072 | SPAHYYGGMDV | 1020 |
| BCMA-3 | GFTFDDY | 1053 | SWNSGS | 1073 | HSFLAY | 1021 |
| BCMA-4 | GFALSNH | 1051 | VYSGS | 1071 | HGGESDV | 1019 |
| BCMA-5 | GYIFDNF | 1054 | NPKNNN | 1074 | GPYYYQSYMDV | 1022 |
| BCMA-6 | GFTFSSD | 1055 | SGSGGT | 1075 | LDSSGYYYARGPRY | 1023 |
| BCMA-7 | GYTFSNY | 1056 | SAYNGN | 1076 | GPYYYYMDV | 1024 |
| BCMA-8 | GFALSNH | 1051 | VYSGS | 1071 | HGGESDV | 1019 |
| BCMA-9 | GFALSNH | 1051 | VYSGS | 1071 | HGGESDV | 1019 |
| BCMA-10 | GFALSNH | 1051 | VYSGS | 1071 | HGGESDV | 1019 |
| BCMA-11 | GFTFSDY | 1057 | SSSGST | 1077 | ESGDGMDV | 1025 |
| BCMA-12 | GFTFSDY | 1057 | SSSGNT | 1078 | STMVREDY | 1026 |
| BCMA-13 | GFALSNH | 1051 | VYSGS | 1071 | HGGESDV | 1019 |
| BCMA-14 | GFALSNH | 1051 | VYSGS | 1071 | HGGESDV | 1019 |
| BCMA-15 | GFALSNH | 1051 | VYSGS | 1071 | HGGESDV | 1019 |
| BCMA-16 | GGSISSSY | 1058 | YYSGS | 1079 | HWQEVVPDAFDI | 1027 |
| | Y | | | | | |
| BCMA-17 | GFSLRTSG | 1059 | DWDED | 1080 | SGAGGTSATAFDI | 1028 |
| | M | | | | | |
| BCMA-18 | GFTFSSY | 1060 | SSSSSY | 1081 | TIAAVYAFDI | 1029 |
| BCMA-19 | GFTFSDY | 1057 | SSSGST | 1077 | DLRGAFDI | 1030 |
| BCMA-20 | GYTVTSH | 1061 | NPSGGV | 1082 | EGSGSGWYFDF | 1031 |
| BCMA-21 | GGSISSGG | 1062 | YYSGS | 1079 | AGIAARLRGAFDI | 1032 |
| | Y | | | | | |
| BCMA-22 | GGTFSSY | 1063 | IPIFGT | 1083 | RGGYQLLRWDVGLLRSAF | 1033 |
| | | | | | DI | |
| BCMA 23 | GDSVSSN | 1064 | YYRSKWY | 1084 | SSPEGLFLYWFDP | 1034 |
| | SA | | | | | |
| BCMA-24 | GFTFSSY | 1060 | SGSGGS | 1085 | VEGSGSLDY | 1035 |
| BCMA-25 | GITFSRY | 1065 | SDSGVS | 1086 | RAGSEASDI | 1036 |
| BCMA-26 | GFTFSSY | 1060 | SGSGGS | 1085 | ATYKRELRYYYGMDV | 1037 |
| BCMA-27 | GFTFSSY | 1060 | SGSGGS | 1085 | ATYKRELRYYYGMDV | 1037 |
| BCMA-28 | GFTFDDY | 1053 | SWNSGS | 1073 | VGKAVPDV | 1038 |
| BCMA-29 | GFTFDDY | 1053 | NWKGNS | 1087 | HQGVAYYNYAMDV | 1039 |
| BCMA-30 | GFTFSSY | 1060 | SGSGGS | 1085 | VVRDGMDV | 1040 |
| BCMA-31 | GFTFSSY | 1060 | SGSGGS | 1085 | IPQTGTFDY | 1041 |
| BCMA-32 | GFTFSSY | 1060 | SGSGGS | 1085 | ANYKRELRYYYGMDV | 1042 |
| BCMA-33 | GFSFSSY | 1066 | SGSGGS | 1085 | ALVGATGAFDI | 1043 |
| BCMA-34 | GFTFSSY | 1060 | SGSGGS | 1085 | WFGEGFDP | 1044 |
| BCMA-35 | GFTFSSY | 1060 | SGSGGS | 1085 | VGYDSSGYYRDYYGMDV | 1045 |
| BCMA-36 | GFTFSSY | 1060 | SGSGGS | 1085 | MGWSSGYLGAFDI | 1046 |
| BCMA-37 | GYTFTNF | 1067 | NTYTGE | 1088 | GEIYYGYDGGFAY | 1047 |
| BCMA-38 | GYTFTDY | 1068 | NTETRE | 1089 | DYSYAMDY | 1048 |
| BCMA-39 | GYTFRHY | 1069 | NTESGV | 1090 | DYLYSLDF | 1049 |
| BCMA-40 | GYTFTHY | 1070 | NTETGE | 1091 | DYLYSCDY | 1050 |

TABLE 16G
BCMA Binders - Heavy chain CDR sequences according to combination of Kabat
and Chothia numbering schemes

| | | SEQ ID | | SEQ ID | | SEQ ID |
|---|---|---|---|---|---|---|
| Antibody | CDR-H1 | NO: | CDR-H2 | NO: | CDR-H3 | NO: |
| BCMA-1 | GFALSNHGM | 1092 | GIVYSGSTYYAASV | 997 | HGGESDV | 1019 |
| | S | | KG | | | |
| BCMA-2 | GFTFSNYAM | 1093 | GISRSGENTYYADS | 998 | SPAHYYGGMDV | 1020 |
| | S | | VKG | | | |
| BCMA-3 | GFTFDDYAM | 1094 | GISWNSGSIGYADS | 999 | HSFLAY | 1021 |
| | H | | VKG | | | |
| BCMA-4 | GFALSNHGM | 1092 | GIVYSGSTYYAASV | 997 | HGGESDV | 1019 |
| | S | | KG | | | |
| BCMA-5 | GYIFDNFGIN | 1095 | WINPKNNNTNYAQK | 1000 | GPYYYQSYMDV | 1022 |
| | | | FQG | | | |
| BCMA-6 | GFTFSSDAM | 1096 | VISGSGGTTYYADS | 1001 | LDSSGYYYARGPRY | 1023 |
| | T | | VKG | | | |
| BCMA-7 | GYTFSNYGI | 1097 | WISAYNGNTNYAQK | 1002 | GPYYYYMDV | 1024 |
| | T | | FQG | | | |
| BCMA-8 | GFALSNHGM | 1092 | GIVYSGSTYYAASV | 997 | HGGESDV | 1019 |
| | S | | KG | | | |
| BCMA-9 | GFALSNHGM | 1092 | GIVYSGSTYYAASV | 997 | HGGESDV | 1019 |
| | S | | KG | | | |
| BCMA-10 | GFALSNHGM | 1092 | GIVYSGSTYYAASV | 997 | HGGESDV | 1019 |
| | S | | KG | | | |
| BCMA-11 | GFTFSDYYM | 1098 | YISSSGSTIYYADSV | 1003 | ESGDGMDV | 1025 |
| | S | | KG | | | |
| BCMA-12 | GFTFSDYYM | 1098 | YISSSGNTIYYADSV | 1004 | STMVREDY | 1026 |
| | S | | KG | | | |
| BCMA-13 | GFALSNHGM | 1092 | GIVYSGSTYYAASV | 997 | HGGESDV | 1019 |
| | S | | KG | | | |
| BCMA-14 | GFALSNHGM | 1092 | GIVYSGSTYYAASV | 997 | HGGESDV | 1019 |
| | S | | KG | | | |
| BCMA-15 | GFALSNHGM | 1092 | GIVYSGSTYYAASV | 997 | HGGESDV | 1019 |
| | S | | KG | | | |
| BCMA-16 | GGSISSSYY | 1099 | SIYYSGSAYYNPSL | 1005 | HWQEWPDAFDI | 1027 |
| | YWG | | KS | | | |
| BCMA-17 | GFSLRTSGM | 1100 | RIDWDEDKFYSTSL | 1006 | SGAGGTSATAFDI | 1028 |
| | CVS | | KT | | | |
| BCMA-18 | GFTFSSYSM | 1101 | SISSSSSYIYYADSV | 1007 | TIAAVYAFDI | 1029 |
| | N | | KG | | | |
| BCMA-19 | GFTFSDYYM | 1098 | YISSSGSTIYYADSV | 1003 | DLRGAFDI | 1030 |
| | S | | KG | | | |
| BCMA-20 | GYTVTSHYI | 1102 | MINPSGGVTAYSQT | 1008 | EGSGSGWYFDF | 1031 |
| | H | | LQG | | | |
| BCMA-21 | GGSISSGGY | 1103 | YIYYSGSTYYNPSL | 1009 | AGIAARLRGAFDI | 1032 |
| | YWS | | KS | | | |
| BCMA-22 | GGTFSSYAI | 1104 | GIIPIFGTANYAQKF | 1010 | RGGYQLLRWDVGLL | 1033 |
| | S | | QG | | RSAFDI | |
| BCMA-23 | GDSVSSNSA | 1105 | RTYYRSKWYSFYAI | 1011 | SSPEGLFLYWFDP | 1034 |
| | AWN | | SLKS | | | |
| BCMA-24 | GFTFSSYAM | 1106 | AISGSGGSTYYADS | 1012 | VEGSGSLDY | 1035 |
| | S | | VKG | | | |
| BCMA-25 | GITFSRYPM | 1107 | GISDSGVSTYYADS | 1013 | RAGSEASDI | 1036 |
| | S | | AKG | | | |
| BCMA-26 | GFTFSSYAM | 1106 | AISGSGGSTYYADS | 1012 | ATYKRELRYYYGMD | 1037 |
| | S | | VKG | | V | |
| BCMA-27 | GFTFSSYAM | 1106 | AISGSGGSTYYADS | 1012 | ATYKRELRYYYGMD | 1037 |
| | S | | VKG | | V | |
| BCMA-28 | GFTFDDYAM | 1094 | GISWNSGSIGYADS | 999 | VGKAVPDV | 1038 |
| | H | | VKG | | | |
| BCMA-29 | GFTFDDYAM | 1094 | SINWKGNSLAYGDS | 1014 | HQGVAYYNYAMDV | 1039 |
| | H | | VKG | | | |
| BCMA-30 | GFTFSSYAM | 1106 | AISGSGGSTYYADS | 1012 | VVRDGMDV | 1040 |
| | S | | VKG | | | |
| BCMA-31 | GFTFSSYAM | 1106 | AISGSGGSTYYADS | 1012 | IPQTGTFDY | 1041 |
| | S | | VKG | | | |
| BCMA-32 | GFTFSSYAM | 1106 | AISGSGGSTYYADS | 1012 | ANYKRELRYYYGMD | 1042 |
| | S | | VKG | | V | |
| BCMA-33 | GFSFSSYAM | 1108 | AISGSGGSTYYADS | 1012 | ALVGATGAFDI | 1043 |
| | S | | VKG | | | |
| BCMA-34 | GFTFSSYAM | 1106 | AISGSGGSTYYADS | 1012 | WFGEGFDP | 1044 |
| | S | | VKG | | | |
| BCMA-35 | GFTFSSYAM | 1106 | AISGSGGSTYYADS | 1012 | VGYDSSGYYRDYYG | 1045 |
| | S | | VKG | | MDV | |
| BCMA-36 | GFTFSSYAM | 1106 | AISGSGGSTYYADS | 1012 | MGWSSGYLGAFDI | 1046 |
| | S | | VKG | | | |
| BCMA-37 | GYTFTNFGM | 1109 | WINTYTGESYFADD | 1015 | GEIYYGYDGGFAY | 1047 |
| | N | | FKG | | | |
| BCMA-38 | GYTFTDYSIN | 1110 | WINTETREPAYAYD | 1016 | DYSYAMDY | 1048 |
| | | | FRG | | | |
| BCMA-39 | GYTFRHYSM | 1111 | RINTESGVPIYADDF | 1017 | DYLYSLDF | 1049 |
| | N | | KG | | | |
| BCMA-40 | GYTFTHYSM | 1112 | RINTETGEPLYADD | 1018 | DYLYSCDY | 1050 |
| | N | | FKG | | | |

In some embodiments, a BCMA ABM comprises the CDR sequences of any one of BCMA-1 to BCMA-40. In some embodiments, the ABM comprises the CDR sequences of BCMA-1. In some embodiments, the ABM comprises the CDR sequences of BCMA-2. In some embodiments, the ABM comprises the CDR sequences of BCMA-3. In some embodiments, the ABM comprises the CDR sequences of BCMA-4. In some embodiments, the ABM comprises the CDR sequences of BCMA-5. In some embodiments, the ABM comprises the CDR sequences of BCMA-6. In some embodiments, the ABM comprises the CDR sequences of BCMA-7. In some embodiments, the ABM comprises the CDR sequences of BCMA-8. In some embodiments, the ABM comprises the CDR sequences of BCMA-9. In some embodiments, the ABM comprises the CDR sequences of BCMA-10. In some embodiments, the ABM comprises the CDR sequences of BCMA-11. In some embodiments, the ABM comprises the CDR sequences of BCMA-12. In some embodiments, the ABM comprises the CDR sequences of BCMA-13. In some embodiments, the ABM comprises the CDR sequences of BCMA-14. In some embodiments, the ABM comprises the CDR sequences of BCMA-15. In some embodiments, the ABM comprises the CDR sequences of BCMA-16. In some embodiments, the ABM comprises the CDR sequences of BCMA-17. In some embodiments, the ABM comprises the CDR sequences of BCMA-18. In some embodiments, the ABM comprises the CDR sequences of BCMA-19. In some embodiments, the ABM comprises the CDR sequences of BCMA-20. In some embodiments, the ABM comprises the CDR sequences of BCMA-21. In some embodiments, the ABM comprises the CDR sequences of BCMA-22. In some embodiments, the ABM comprises the CDR sequences of BCMA-23. In some embodiments, the ABM comprises the CDR sequences of BCMA-24. In some embodiments, the ABM comprises the CDR sequences of BCMA-25. In some embodiments, the ABM comprises the CDR sequences of BCMA-26. In some embodiments, the ABM comprises the CDR sequences of BCMA-27. In some embodiments, the ABM comprises the CDR sequences of BCMA-28. In some embodiments, the ABM comprises the CDR sequences of BCMA-29. In some embodiments, the ABM comprises the CDR sequences of BCMA-30. In some embodiments, the ABM comprises the CDR sequences of BCMA-31. In some embodiments, the ABM comprises the CDR sequences of BCMA-32. In some embodiments, the ABM comprises the CDR sequences of BCMA-33. In some embodiments, the ABM comprises the CDR sequences of BCMA-34. In some embodiments, the ABM comprises the CDR sequences of BCMA-35. In some embodiments, the ABM comprises the CDR sequences of BCMA-36. In some embodiments, the ABM comprises the CDR sequences of BCMA-37. In some embodiments, the ABM comprises the CDR sequences of BCMA-38. In some embodiments, the ABM comprises the CDR sequences of BCMA-39. In some embodiments, the ABM comprises the CDR sequences of BCMA-40.

In some embodiments, the CDRs are defined by Kabat numbering, as set forth in Tables 16B and 16E. In other embodiments, the CDRs are defined by Chothia numbering, as set forth in Tables 16C and 16F. In yet other embodiments, the CDRs are defined by a combination of Kabat and Chothia numbering, as set forth in Tables 16D and 16G.

In some embodiments, a MBM (e.g., TBM) comprising a BCMA ABM can comprise the heavy and light chain variable sequences of any of BCMA-1 to BCMA-40, as set forth in Table 16A.

In some embodiments, the ABM comprises the heavy and light chain variable sequences of BCMA-1. In some embodiments, the ABM comprises the heavy and light chain variable sequences of BCMA-2. In some embodiments, the ABM comprises the heavy and light chain variable sequences of BCMA-3. In some embodiments, the ABM comprises the heavy and light chain variable sequences of BCMA-4. In some embodiments, the ABM comprises the heavy and light chain variable sequences of BCMA-5. In some embodiments, the ABM comprises the heavy and light chain variable sequences of BCMA-6. In some embodiments, the ABM comprises the heavy and light chain variable sequences of BCMA-7. In some embodiments, the ABM comprises the heavy and light chain variable sequences of BCMA-8. In some embodiments, the ABM comprises the heavy and light chain variable sequences of BCMA-9. In some embodiments, the ABM comprises the heavy and light chain variable sequences of BCMA-10. In some embodiments, the ABM comprises the heavy and light chain variable sequences of BCMA-11. In some embodiments, the ABM comprises the heavy and light chain variable sequences of BCMA-12. In some embodiments, the ABM comprises the heavy and light chain variable sequences of BCMA-13. In some embodiments, the ABM comprises the heavy and light chain variable sequences of BCMA-14. In some embodiments, the ABM comprises the heavy and light chain variable sequences of BCMA-15. In some embodiments, the ABM comprises the heavy and light chain variable sequences of BCMA-16. In some embodiments, the ABM comprises the heavy and light chain variable sequences of BCMA-17. In some embodiments, the ABM comprises the heavy and light chain variable sequences of BCMA-18. In some embodiments, the ABM comprises the heavy and light chain variable sequences of BCMA-19. In some embodiments, the ABM comprises the heavy and light chain variable sequences of BCMA-20. In some embodiments, the ABM comprises the heavy and light chain variable sequences of BCMA-21. In some embodiments, the ABM comprises the heavy and light chain variable sequences of BCMA-22.

In some embodiments, the ABM comprises the heavy and light chain variable sequences of BCMA-23. In some embodiments, the ABM comprises the heavy and light chain variable sequences of BCMA-24. In some embodiments, the ABM comprises the heavy and light chain variable sequences of BCMA-25. In some embodiments, the ABM comprises the heavy and light chain variable sequences of BCMA-26. In some embodiments, the ABM comprises the heavy and light chain variable sequences of BCMA-27. In some embodiments, the ABM comprises the heavy and light chain variable sequences of BCMA-28. In some embodiments, the ABM comprises the heavy and light chain variable sequences of BCMA-29. In some embodiments, the ABM comprises the heavy and light chain variable sequences of BCMA-30. In some embodiments, the ABM comprises the heavy and light chain variable sequences of BCMA-31. In some embodiments, the ABM comprises the heavy and light chain variable sequences of BCMA-32. In some embodiments, the ABM comprises the heavy and light chain variable sequences of BCMA-33. In some embodiments, the ABM comprises the heavy and light chain variable sequences of BCMA-34. In some embodiments, the ABM comprises the heavy and light chain variable sequences of BCMA-35. In some embodiments, the ABM comprises the heavy and light chain variable sequences of BCMA-36. In some embodiments, the ABM comprises the heavy and light chain variable sequences of BCMA-37. In some embodiments, the ABM comprises the heavy and light chain variable sequences of BCMA-38. In some embodiments, the ABM comprises the heavy and light chain variable sequences of BCMA-39. In some embodiments, the ABM comprises the heavy and light chain variable sequences of BCMA-40.

7.10.2. CD19

B cells express cell surface proteins which can be utilized as markers for differentiation and identification. One such human B-cell marker is a CD19 antigen and is found on mature B cells but not on plasma cells. CD19 is expressed during early pre-B cell development and remains until plasma cell differentiation. CD19 is expressed on both normal B cells and malignant B cells whose abnormal growth can lead to B-cell lymphomas. For example, CD19 is expressed on B-cell lineage malignancies, including, but not limited to non-Hodgkin's lymphoma (B-NHL), chronic lymphocytic leukemia, and acute lymphoblastic leukemia.

In certain aspects, a MBM comprises an ABM2 or ABM3 that specifically binds to CD19 (such ABMs are referred to as “CD19 ABMs” for convenience). Exemplary CDR and variable domain sequences that can be incorporated into CD19 ABMs are set forth in Table 17 below.

TABLE 17
CD19 Binders

| | | | SEQ |
|---|---|---|---|
| | | | ID |
| Name | Domain | Sequence | NO: |
| CD19-H1 | CDR-H1 | DYGVS | 104 |
| CD19-H2A | CDR-H2 | VIWGSETTYYNSALKS | 105 |
| CD19-H2B | CDR-H2 | VIWGSETTYYSSSLKS | 106 |
| CD19-H2C | CDR-H2 | VIWGSETTYYQSSLKS | 107 |
| CD19-H2D | CDR-H2 | VIWGSETTYYNSSLKS | 108 |
| CD19-H3 | CDR-H3 | HYYYGGSYAMDY | 109 |
| CD19-L1 | CDR-L1 | RASQDISKYLN | 110 |
| CD19-L2 | CDR-L2 | HTSRLHS | 111 |
| CD19-L3 | CDR-L3 | QQGNTLPYT | 112 |
| CD19-VHA | VH | EVKLQESGPGLVAPSQSLSVTCTVSGVSLPDYGVSWI | 113 |
| | | RQPPRKGLEWLGVIWGSETTYYNSALKSRLTIIKDNSK | |
| | | SQVFLKMNSLQTDDTAIYYCAKHYYYGGSYAMDYWG | |
| | | QGTSVTVSS | |
| CD19-VHB | VH | QVQLQESGPGLVKPSETLSLTCTVSGVSLPDYGVSWIR | 114 |
| | | QPPGKGLEWIGVIWGSETTYYSSSLKSRVTISKDNSKN | |
| | | QVSLKLSSVTAADTAVYYCAKHYYYGGSYAMDYWGQ | |
| | | GTLVTVSS | |
| CD19-VHC | VH | QVQLQESGPGLVKPSETLSLTCTVSGVSLPDYGVSWIR | 115 |
| | | QPPGKGLEWIGVIWGSETTYYQSSLKSRVTISKDNSKN | |
| | | QVSLKLSSVTAADTAVYYCAKHYYYGGSYAMDYWGQ | |
| | | GTLVTVSS | |
| CD19-VHD | VH | QVQLQESGPGLVKPSETLSLTCTVSGVSLPDYGVSWIR | 116 |
| | | QPPGKGLEWIGVIWGSETTYYNSSLKSRVTISKDNSKN | |
| | | QVSLKLSSVTAADTAVYYCAKHYYYGGSYAMDYWGQ | |
| | | GTLVTVSS | |
| CD19-VLA | VL | DIQMTQTTSSLSASLGDRVTISCRASQDISKYLNWYQQ | 117 |
| | | KPDGTVKLLIYHTSRLHSGVPSRFSGSGSGTDYSLTISN | |
| | | LEQEDIATYFCQQGNTLPYTFGGGTKLEIT | |
| CD19-VLB | VL | EIVMTQSPATLSLSPGERATLSCRASQDISKYLNWYQQ | 118 |
| | | KPGQAPRLLIYHTSRLHSGIPARFSGSGSGTDYTLTISS | |
| | | LQPEDFAVYFCQQGNTLPYTFGQGTKLEIK | |
| CD19-scFv1 | scFv | EIVMTQSPATLSLSPGERATLSCRASQDISKYLNWYQQ | 119 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVSL | |
| | | PDYGVSWIRQPPGKGLEWIGVIWGSETTYYSSSLKSR | |
| | | VTISKDNSKNQVSLKLSSVTAADTAVYYCAKHYYYGGS | |
| | | YAMDYWGQGTLVTVSS | |
| CD19-scFv2 | scFv | EIVMTQSPATLSLSPGERATLSCRASQDISKYLNWYQQ | 120 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVSL | |
| | | PDYGVSWIRQPPGKGLEWIGVIWGSETTYYQSSLKSR | |
| | | VTISKDNSKNQVSLKLSSVTAADTAVYYCAKHYYYGGS | |
| | | YAMDYWGQGTLVTVSS | |
| CD19-scFv3 | scFv | QVQLQESGPGLVKPSETLSLTCTVSGVSLPDYGVSWIR | 121 |
| | | QPPGKGLEWIGVIWGSETTYYSSSLKSRVTISKDNSKN | |
| | | QVSLKLSSVTAADTAVYYCAKHYYYGGSYAMDYWGQ | |
| | | GTLVTVSSGGGGSGGGGSGGGGSEIVMTQSPATLSLS | |
| | | PGERATLSCRASQDISKYLNVVYQQKPGQAPRLLIYHTS | |
| | | RLHSGIPARFSGSGSGTDYTLTISSLQPEDFAVYFCQQ | |
| | | GNTLPYTFGQGTKLEK | |
| CD19-scFv4 | scFv | QVQLQESGPGLVKPSETLSLTCTVSGVSLPDYGVSWIR | 122 |
| | | QPPGKGLEWIGVIWGSETTYYQSSLKSRVTISKDNSKN | |
| | | QVSLKLSSVTAADTAVYYCAKHYYYGGSYAMDYWGQ | |
| | | GTLVTVSSGGGGSGGGGSGGGGSEIVMTQSPATLSLS | |
| | | PGERATLSCRASQDISKYLNVVYQQKPGQAPRLLIYHTS | |
| | | RLHSGIPARFSGSGSGTDYTLTISSLQPEDFAVYFCQQ | |
| | | GNTLPYTFGQGTKLEIK | |
| CD19-scFv5 | scFv | EIVMTQSPATLSLSPGERATLSCRASQDISKYLNWYQQ | 123 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYS | |
| | | SSLKSRVTISKDNSKNQVSLKLSSVTAADTAVYYCAKH | |
| | | YYYGGSYAMDYWGQGTLVTVSS | |
| CD19-scFv6 | scFv | EIVMTQSPATLSLSPGERATLSCRASQDISKYLNWYQQ | 124 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYQ | |
| | | SSLKSRVTISKDNSKNQVSLKLSSVTAADTAVYYCAKH | |
| | | YYYGGSYAMDYWGQGTLVTVSS | |
| CD19-scFv7 | scFv | QVQLQESGPGLVKPSETLSLTCTVSGVSLPDYGVSWIR | 125 |
| | | QPPGKGLEWIGVIWGSETTYYSSSLKSRVTISKDNSKN | |
| | | QVSLKLSSVTAADTAVYYCAKHYYYGGSYAMDYWGQ | |
| | | GTLVTVSSGGGGSGGGGSGGGGSGGGGSEIVMTQS | |
| | | PATLSLSPGERATLSCRASQDISKYLNVVYQQKPGQAP | |
| | | RLLIYHTSRLHSGIPARFSGSGSGTDYTLTISSLQPEDF | |
| | | AVYFCQQGNTLPYTFGQGTKLEIK | |
| CD19-scFv8 | scFv | QVQLQESGPGLVKPSETLSLTCTVSGVSLPDYGVSWIR | 126 |
| | | QPPGKGLEWIGVIWGSETTYYQSSLKSRVTISKDNSKN | |
| | | QVSLKLSSVTAADTAVYYCAKHYYYGGSYAMDYWGQ | |
| | | GTLVTVSSGGGGSGGGGSGGGGSGGGGSEIVMTQS | |
| | | PATLSLSPGERATLSCRASQDISKYLNVVYQQKPGQAP | |
| | | RLLIYHTSRLHSGIPARFSGSGSGTDYTLTISSLQPEDF | |
| | | AVYFCQQGNTLPYTFGQGTKLEIK | |
| CD19-scFv9 | scFv | EIVMTQSPATLSLSPGERATLSCRASQDISKYLNWYQQ | 127 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYN | |
| | | SSLKSRVTISKDNSKNQVSLKLSSVTAADTAVYYCAKH | |
| | | YYYGGSYAMDYWGQGTLVTVSS | |
| CD19-scFv10 | scFv | QVQLQESGPGLVKPSETLSLTCTVSGVSLPDYGVSWIR | 128 |
| | | QPPGKGLEWIGVIWGSETTYYNSSLKSRVTISKDNSKN | |
| | | QVSLKLSSVTAADTAVYYCAKHYYYGGSYAMDYWGQ | |
| | | GTLVTVSSGGGGSGGGGSGGGGSGGGGSEIVMTQS | |
| | | PATLSLSPGERATLSCRASQDISKYLNVVYQQKPGQAP | |
| | | RLLIYHTSRLHSGIPARFSGSGSGTDYTLTISSLQPEDF | |
| | | AVYFCQQGNTLPYTFGQGTKLEIK | |
| CD19-scFv11 | scFv | EIVMTQSPATLSLSPGERATLSCRASQDISKYLNWYQQ | 129 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVSL | |
| | | PDYGVSWIRQPPGKGLEWIGVIWGSETTYYNSSLKSR | |
| | | VTISKDNSKNQVSLKLSSVTAADTAVYYCAKHYYYGGS | |
| | | YAMDYWGQGTLVTVSS | |
| CD19-scFv12 | scFv | QVQLQESGPGLVKPSETLSLTCTVSGVSLPDYGVSWIR | 130 |
| | | QPPGKGLEWIGVIWGSETTYYNSSLKSRVTISKDNSKN | |
| | | QVSLKLSSVTAADTAVYYCAKHYYYGGSYAMDYWGQ | |
| | | GTLVTVSSGGGGSGGGGSGGGGSEIVMTQSPATLSLS | |
| | | PGERATLSCRASQDISKYLNVVYQQKPGQAPRLLIYHTS | |
| | | RLHSGIPARFSGSGSGTDYTLTISSLQPEDFAVYFCQQ | |
| | | GNTLPYTFGQGTKLEIK | |

In certain aspects, a CD19 ABM comprises heavy chain CDRs having the amino acid sequences of CD19-H1, CD19-H2A, and CD19-H3 as set forth in Table 17 and light chain CDRs having the amino acid sequences of CD19-L1, CD19-L2, and CD19-L3 as set forth in Table 17. In a specific embodiment, the ABM comprises a heavy chain variable region having the amino acid sequences of VHA as set forth in Table 17 and a light chain variable region having the amino acid sequences of VLA as set forth in Table 17.

In other aspects, the ABM comprises heavy chain CDRs having the amino acid sequences of CD19-H1, CD19-H2B, and CD19-H3 as set forth in Table 17 and light chain CDRs having the amino acid sequences of CD19-L1, CD19-L2, and CD19-L3 as set forth in Table 17. In a specific embodiment, the ABM comprises a heavy chain variable region having the amino acid sequences of VHB as set forth in Table 17 and a light chain variable region having the amino acid sequences of VLB as set forth in Table 17.

In further aspects, the ABM comprises heavy chain CDRs having the amino acid sequences of CD19-H1, CD19-H2C, and CD19-H3 as set forth in Table 17 and light chain CDRs having the amino acid sequences of CD19-L1, CD19-L2, and CD19-L3 as set forth in Table 17. In a specific embodiment, ABM comprises a heavy chain variable region having the amino acid sequences of VHC as set forth in Table 17 and a light chain variable region having the amino acid sequences of VLB as set forth in Table 17.

In further aspects, the ABM comprises heavy chain CDRs having the amino acid sequences of CD19-H1, CD19-H2D, and CD19-H3 as set forth in Table 17 and light chain CDRs having the amino acid sequences of CD19-L1, CD19-L2, and CD19-L3 as set forth in Table 17. In a specific embodiment, the ABM comprises a heavy chain variable region having the amino acid sequences of VHD as set forth in Table 17 and a light chain variable region having the amino acid sequences of VLB as set forth in Table 17.

In yet further aspects, the ABM is in the form of an scFV. Exemplary anti-CD19 scFvs comprise the amino acid sequence of any one of CD19-scFv1 through CD19-scFv12 as set forth in Table 17.

### 7.11. Nucleic Acids and Host Cells

In another aspect, the disclosure provides nucleic acids encoding the CD3 binding molecules (e.g., MBMs) of the disclosure. In some embodiments, the CD3 binding molecules (e.g., MBMs) are encoded by a single nucleic acid. In other embodiments, the CD3 binding molecules (e.g., MBMs) are encoded by a plurality (e.g., two, three, four or more) nucleic acids.

A single nucleic acid can encode a CD3 binding molecule (e.g., MBM) that comprises a single polypeptide chain, a CD3 binding molecule (e.g., MBM) that comprises two or more polypeptide chains, or a portion of a CD3 binding molecule (e.g., MBM) that comprises more than two polypeptide chains (for example, a single nucleic acid can encode two polypeptide chains of a CD3 binding molecule (e.g., MBM) comprising three, four or more polypeptide chains, or three polypeptide chains of a CD3 binding molecule (e.g., MBM) comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a CD3 binding molecule (e.g., MBM) comprising two or more polypeptide chains is encoded by two or more nucleic acids. The number of nucleic acids encoding a CD3 binding molecule (e.g., MBM) can be equal to or less than the number of polypeptide chains in the CD3 binding molecule (e.g., MBM) (for example, when more than one polypeptide chains are encoded by a single nucleic acid).

The nucleic acids can be DNA or RNA (e.g., mRNA).

In another aspect, the disclosure provides host cells and vectors containing the nucleic acids of the disclosure. The nucleic acids can be present in a single vector or separate vectors present in the same host cell or separate host cell, as described in more detail herein below.

7.11.1. Vectors

The disclosure provides vectors comprising nucleotide sequences encoding a CD3 binding molecule (e.g., MBM) or a CD3 binding molecule (e.g., MBM) component described herein. In one embodiment, the vectors comprise nucleotides encoding an immunoglobulin-based ABM described herein. In one embodiment, the vectors comprise nucleotides encoding an Fc domain described herein. In one embodiment, the vectors comprise nucleotides encoding a recombinant non-immunoglobulin based ABM described herein. A vector can encode one or more ABMs, one or more Fc domains, one or more non-immunoglobulin based ABM, or a combination thereof (e.g., when multiple components or sub-components are encoded as a single polypeptide chain). In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker can provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements can also be needed for optimal synthesis of mRNA. These elements can include splice signals, as well as transcriptional promoters, enhancers, and termination signals.

Once the expression vector or DNA sequence containing the constructs has been prepared for expression, the expression vectors can be transfected or introduced into an appropriate host cell. Various techniques can be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and can be varied or optimized depending upon the specific expression vector and mammalian host cell employed, based upon the present description.

7.11.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes can include a promoter, an open reading frame with or without introns, and a termination signal. Additional factors necessary or helpful in effecting expression can also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 7.12. CD3 Binding Molecules with Extended In Vivo Half-Life

The CD3 binding molecules can be modified to have an extended half-life in vivo.

A variety of strategies can be used to extend the half life of CD3 binding molecules of the disclosure. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nanocarriers, slow release formulations, or medical devices.

To prolong the serum circulation of CD3 binding molecules in vivo, inert polymer molecules such as high molecular weight PEG can be attached to the CD3 binding molecules with or without a multifunctional linker either through site-specific conjugation of the PEG to the N- or C-terminus of a polypeptide comprising the CD3 binding molecule or via epsilon-amino groups present on lysine residues. To pegylate a CD3 binding molecule, the molecule can be reacted with polyethylene glycol (PEG), such as a reactive ester or aldehyde derivative of PEG, under conditions in which one or more PEG groups become attached to the CD3 binding molecules. The pegylation can be carried out by an acylation reaction or an alkylation reaction with a reactive PEG molecule (or an analogous reactive water-soluble polymer). As used herein, the term “polyethylene glycol” is intended to encompass any one of the forms of PEG that have been used to derivatize other proteins, such as mono (C1-C10)alkoxy- or aryloxy-polyethylene glycol or polyethylene glycol-maleimide. In one embodiment, the CD3 binding molecule to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known and can be applied to CD3 binding molecules of the disclosure. See for example, EP 0154316 by Nishimura et al. and EP 0401384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a normative amino acid any place an amber codon is positioned, converting the amber from a stop codon to one that signals incorporation of the chemically specified amino acid.

Recombinant pegylation technology (rPEG) can also be used for serum half life extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum half life of the protein is greatly increased. In contrast to traditional PEGylation, which requires chemical conjugation and repurification, the manufacturing process is greatly simplified and the product is homogeneous.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology include the use of hydroxyethyl starch (“HES”) derivatives linked to CD3 binding molecules. HES is a modified natural polymer derived from waxy maize starch and can be metabolized by the body's enzymes. HES solutions are usually administered to substitute deficient blood volume and to improve the rheological properties of the blood. Hesylation of a CD3 binding molecule enables the prolongation of the circulation half-life by increasing the stability of the molecule, as well as by reducing renal clearance, resulting in an increased biological activity. By varying different parameters, such as the molecular weight of HES, a wide range of HES CD3 binding molecule conjugates can be customized.

CD3 binding molecules having an increased half-life in vivo can also be generated introducing one or more amino acid modifications (i.e., substitutions, insertions or deletions) into an IgG constant domain, or FcRn binding fragment thereof (e.g., an Fc or hinge Fc domain fragment). See, e.g., International Publication No. WO 98/23289; International Publication No. WO 97/34631; and U.S. Pat. No. 6,277,375.

Furthermore, the CD3 binding molecules can be conjugated to albumin, a domain of albumin, an albumin-binding protein, or an albumin-binding antibody or antibody fragments thereof, in order to make the molecules more stable in vivo or have a longer half life in vivo. The techniques are well-known, see, e.g., International Publication Nos. WO 93/15199, WO 93/15200, and WO 01/77137; and European Patent No. EP 413,622.

The CD3 binding molecules of the present disclosure can also be fused to one or more human serum albumin (HSA) polypeptides, or a portion thereof. The use of albumin as a component of an albumin fusion protein as a carrier for various proteins has been suggested in WO 93/15199, WO 93/15200, and EP 413 622. The use of N-terminal fragments of HSA for fusions to polypeptides has also been proposed (EP 399 666). Accordingly, by genetically or chemically fusing or conjugating the molecules to albumin, can stabilize or extend the shelf-life, and/or to retain the molecule's activity for extended periods of time in solution, in vitro and/or in vivo. Additional methods pertaining to HSA fusions can be found, for example, in WO 2001077137 and WO 200306007. In an embodiment, the expression of the fusion protein is performed in mammalian cell lines, for example, CHO cell lines.

The CD3 binding molecules of the present disclosure can also be fused to an antibody or antibody fragment thereof that binds to albumin, e.g., human serum albumin (HSA). The albumin-binding antibody or antibody fragment thereof can be a Fab, a scFv, a Fv, an scFab, a (Fab′)2, a single domain antibody, a camelid VHH domain, a VH or VL domain, or a full-length monoclonal antibody (mAb).

The CD3 binding molecules of the present disclosure can also be fused to a fatty acid to extend their half-life. Fatty acids suitable for linking to a biomolecule have been described in the art, e.g., WO2015/200078, WO2015/191781, US2013/0040884. Suitable half-life extending fatty acids include those defined as a C6-70alkyl, a C6-70alkenyl or a C6-70alkynyl chain, each of which is substituted with at least one carboxylic acid (for example 1, 2, 3 or 4 CO2H) and optionally further substituted with hydroxyl group. For example, the CD3 binding molecules described herein can be linked to a fatty acid having any of the following Formulae A1, A2 or A3:

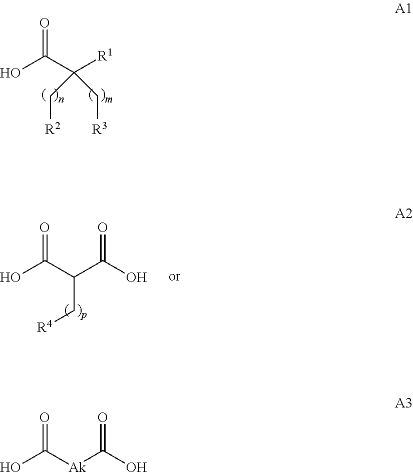

R¹ is CO₂H or H;
R², R³ and R⁴ are independently of each other H, OH, CO₂H, —CH═CH₂ or —C≡CH;
Ak is a branched C₆-C₃₀alkylene;
n, m and p are independently of each other an integer between 6 and 30; or an amide, ester or pharmaceutically acceptable salt thereof.

In some embodiments, the fatty acid is of Formula A1, e.g., a fatty acid of Formula A1 where n and m are independently 8 to 20, e.g., 10 to 16. In another embodiment, the fatty acid moiety is of Formula A1 and where at least one of R² and R³ is CO₂H.

In some embodiments, the fatty acid is selected from the following Formulae:

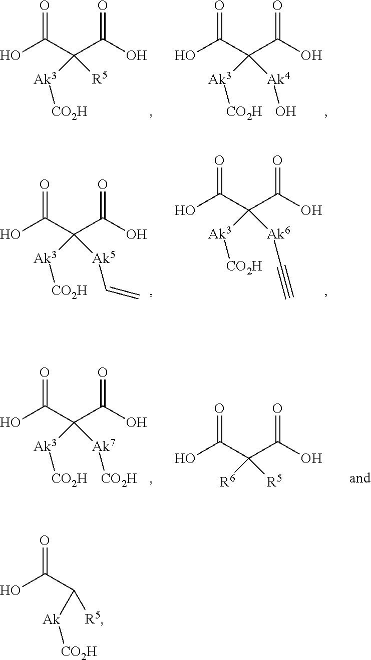

where Ak³, Ak⁴, Ak⁵, Ak⁶ and Ak⁷ are independently a (C₈₋₂₀)alkylene, R⁵ and R⁶ are independently (C₈₋₂₀)alkyl.

In some embodiments, the fatty acid is selected from the following Formulae:

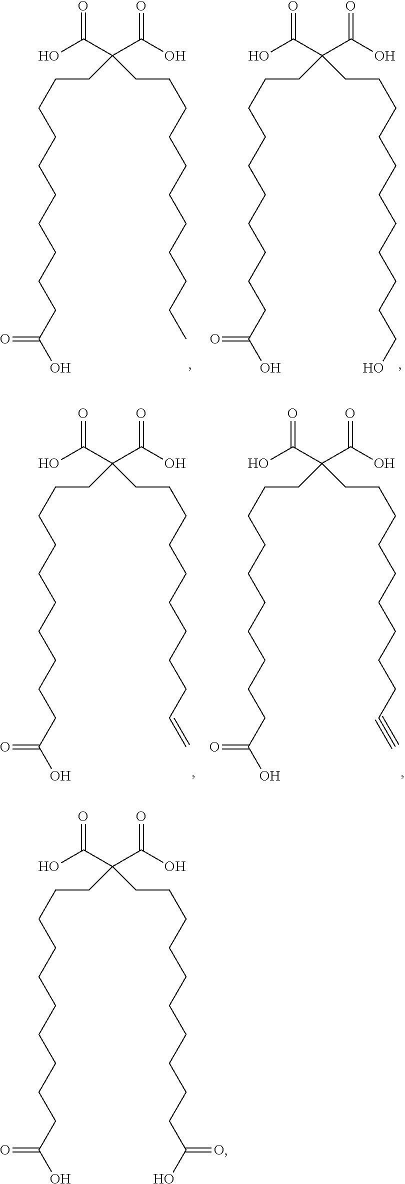

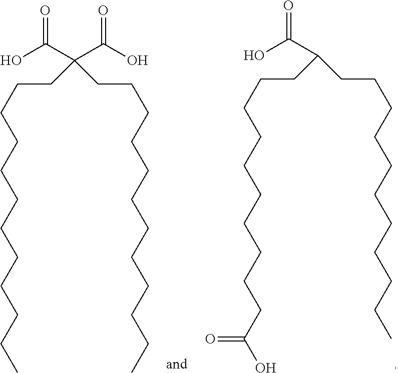 [SMILES: CCCCCCCCCCCCC(CCCCCCCCCCCC)(C(=O)O)C(=O)O ; CCCCCCCCCCCC(CCCCCCCCCCC(=O)O)C(=O)O]

In some embodiments, the fatty acid is selected from the following Formulae:

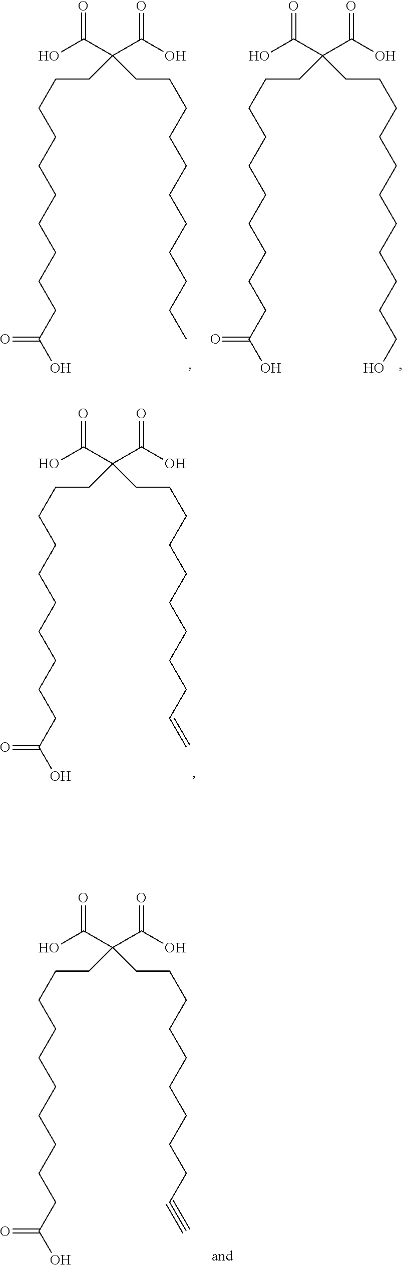

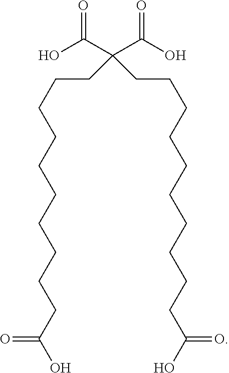 [SMILES: O=C(O)CCCCCCCCCCC(CCCCCCCCCCC(=O)O)(C(=O)O)C(=O)O]

In some embodiments, the fatty acid is of Formula A2 or A3. In a particular embodiment, the conjugate comprises a fatty acid moiety of Formula A2 where p is 8 to 20, or a fatty acid moiety of Formula A3 where Ak is C₈₋₂₀alkylene.

### 7.13. Antibody-Drug Conjugates

The CD3 binding molecules (e.g., MBMs) can be conjugated, e.g., via a linker, to a drug moiety. Such conjugates are referred to herein as antibody-drug conjugates (or “ADCs”) for convenience, notwithstanding the fact that one or more (or all) of the ABMs might be based on non-immunoglobulin scaffolds.

In certain aspects, the drug moiety exerts a cytotoxic or cytostatic activity. In one embodiment, the drug moiety is chosen from a maytansinoid, a kinesin-like protein KIF11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor.

In one embodiment, the linker is chosen from a cleavable linker, a non-cleavable linker, a hydrophilic linker, a procharged linker, or a dicarboxylic acid based linker.

In specific embodiments, the ADCs are compounds according to structural formula (I):

[D-L-XY]ₙ-Ab

or salts thereof, where each “D” represents, independently of the others, a cytotoxic and/or cytostatic agent (“drug”); each “L” represents, independently of the others, a linker; “Ab” represents a MBM described herein; each “XY” represents a linkage formed between a functional group R^{x} on the linker and a “complementary” functional group R^{y} on the antibody, and n represents the number of drugs linked to, or drug-to-antibody ratio (DAR), of the ADC.

Specific embodiments of the various antibodies (Ab) that can comprise the ADCs include the various embodiments of MBMs described above.

In some specific embodiments of the ADCs and/or salts of structural formula (I), each D is the same and/or each L is the same.

Specific embodiments of cytotoxic and/or cytostatic agents (D) and linkers (L) that can comprise the ADCs, as well as the number of cytotoxic and/or cytostatic agents linked to the ADCs, are described in more detail below.

7.13.1. Cytotoxic and/or Cytostatic Agents

The cytotoxic and/or cytostatic agents can be any agents known to inhibit the growth and/or replication of and/or kill cells, and in particular cancer and/or tumor cells. Numerous agents having cytotoxic and/or cytostatic properties are known in the literature. Non-limiting examples of classes of cytotoxic and/or cytostatic agents include, by way of example and not limitation, radionuclides, alkylating agents, topoisomerase I inhibitors, topoisomerase II inhibitors, DNA intercalating agents (e.g., groove binding agents such as minor groove binders), RNA/DNA antimetabolites, cell cycle modulators, kinase inhibitors, protein synthesis inhibitors, histone deacetylase inhibitors, mitochondria inhibitors, and antimitotic agents.

Specific non-limiting examples of agents within certain of these various classes are provided below.

Alkylating Agents: asaley ((L-Leucine, N-[N-acetyl-4-[bis-(2-chloroethyl)amino]-DL-phenylalanyl]-, ethylester; NSC 167780; CAS Registry No. 3577897)); AZQ ((1,4-cyclohexadiene-1,4-dicarbamic acid, 2,5-bis(1-aziridinyl)-3,6-dioxo-, diethyl ester; NSC 182986; CAS Registry No. 57998682)); BCNU ((N,N′-Bis(2-chloroethyl)-N-nitrosourea; NSC 409962; CAS Registry No. 154938)); busulfan (1,4-butanediol dimethanesulfonate; NSC 750; CAS Registry No. 55981); (carboxyphthalato)platinum (NSC 27164; CAS Registry No. 65296813); CBDCA ((cis-(1,1-cyclobutanedicarboxylato)diammineplatinum(II)); NSC 241240; CAS Registry No. 41575944)); CCNU ((N-(2-chloroethyl)-N′-cyclohexyl-N-nitrosourea; NSC 79037; CAS Registry No. 13010474)); CHIP (iproplatin; NSC 256927); chlorambucil (NSC 3088; CAS Registry No. 305033); chlorozotocin ((2-[[[(2-chloroethyl) nitrosoamino]carbonyl]amino]-2-deoxy-D-glucopyranose; NSC 178248; CAS Registry No. 54749905)); cis-platinum (cisplatin; NSC 119875; CAS Registry No. 15663271); clomesone (NSC 338947; CAS Registry No. 88343720); cyanomorpholinodoxorubicin (NCS 357704; CAS Registry No. 88254073); cyclodisone (NSC 348948; CAS Registry No. 99591738); dianhydrogalactitol (5,6-diepoxydulcitol; NSC 132313; CAS Registry No. 23261203); fluorodopan ((5-[(2-chloroethyl)-(2-fluoroethyl)amino]-6-methyl-uracil; NSC 73754; CAS Registry No. 834913); hepsulfam (NSC 329680; CAS Registry No. 96892578); hycanthone (NSC 142982; CAS Registry No. 23255938); melphalan (NSC 8806; CAS Registry No. 3223072); methyl CCNU ((1-(2-chloroethyl)-3-(trans-4-methylcyclohexane)-1-nitrosourea; NSC 95441; 13909096); mitomycin C (NSC 26980; CAS Registry No. 50077); mitozolamide (NSC 353451; CAS Registry No. 85622953); nitrogen mustard ((bis(2-chloroethyl)methylamine hydrochloride; NSC 762; CAS Registry No. 55867); PCNU ((1-(2-chloroethyl)-3-(2,6-dioxo-3-piperidyl)-1-nitrosourea; NSC 95466; CAS Registry No. 13909029)); piperazine alkylator ((1-(2-chloroethyl)-4-(3-chloropropyl)-piperazine dihydrochloride; NSC 344007)); piperazinedione (NSC 135758; CAS Registry No. 41109802); pipobroman ((N,N-bis(3-bromopropionyl) piperazine; NSC 25154; CAS Registry No. 54911)); porfiromycin (N-methylmitomycin C; NSC 56410; CAS Registry No. 801525); spirohydantoin mustard (NSC 172112; CAS Registry No. 56605164); teroxirone (triglycidylisocyanurate; NSC 296934; CAS Registry No. 2451629); tetraplatin (NSC 363812; CAS Registry No. 62816982); thio-tepa (N,N′,N″-tri-1,2-ethanediylthio phosphoramide; NSC 6396; CAS Registry No. 52244); triethylenemelamine (NSC 9706; CAS Registry No. 51183); uracil nitrogen mustard (desmethyldopan; NSC 34462; CAS Registry No. 66751); Yoshi-864 ((bis(3-mesyloxy propyl)amine hydrochloride; NSC 102627; CAS Registry No. 3458228).

Topoisomerase I Inhibitors: camptothecin (NSC 94600; CAS Registry No. 7689-03-4); various camptothecin derivatives and analogs (for example, NSC 100880, NSC 603071, NSC 107124, NSC 643833, NSC 629971, NSC 295500, NSC 249910, NSC 606985, NSC 74028, NSC 176323, NSC 295501, NSC 606172, NSC 606173, NSC 610458, NSC 618939, NSC 610457, NSC 610459, NSC 606499, NSC 610456, NSC 364830, and NSC 606497); morpholinisoxorubicin (NSC 354646; CAS Registry No. 89196043); SN-38 (NSC 673596; CAS Registry No. 86639-52-3).

Topoisomerase II Inhibitors: doxorubicin (NSC 123127; CAS Registry No. 25316409); amonafide (benzisoquinolinedione; NSC 308847; CAS Registry No. 69408817); m-AMSA ((4′-(9-acridinylamino)-3′-methoxymethanesulfonanilide; NSC 249992; CAS Registry No. 51264143)); anthrapyrazole derivative ((NSC 355644); etoposide (VP-16; NSC 141540; CAS Registry No. 33419420); pyrazolo acridine ((pyrazolo[3,4,5-kl]acridine-2(6H)-propanamine, 9-methoxy-N, N-dimethyl-5-nitro-, monomethanesulfonate; NSC 366140; CAS Registry No. 99009219); bisantrene hydrochloride (NSC 337766; CAS Registry No. 71439684); daunorubicin (NSC 821151; CAS Registry No. 23541506); deoxydoxorubicin (NSC 267469; CAS Registry No. 63950061); mitoxantrone (NSC 301739; CAS Registry No. 70476823); menogaril (NSC 269148; CAS Registry No. 71628961); N,N-dibenzyl daunomycin (NSC 268242; CAS Registry No. 70878512); oxanthrazole (NSC 349174; CAS Registry No. 105118125); rubidazone (NSC 164011; CAS Registry No. 36508711); teniposide (VM-26; NSC 122819; CAS Registry No. 29767202).

DNA Intercalating Agents: anthramycin (CAS Registry No. 4803274); chicamycin A (CAS Registry No. 89675376); tomaymycin (CAS Registry No. 35050556); DC-81 (CAS Registry No. 81307246); sibiromycin (CAS Registry No. 12684332); pyrrolobenzodiazepine derivative (CAS Registry No. 945490095); SGD-1882 ((S)-2-(4-aminophenyl)-7-methoxy-8-(3-4(S)-7-methoxy-2-(4-methoxyphenyl)-5-oxo-5,11a-dihydro-1H-benzo[e]pyrrolo[1,2-a][1,4]diazepin-8-yl)oxy)propoxy)-1H-benzo[e]pyrrolo[1,2-a][1,4]diazepin-5(11aH)-one); SG2000 (SJG-136; (11aS,11a′S)-8,8′-(propane-1,3-diylbis(oxy))bis(7-methoxy-2-methylene-2,3-dihydro-1H-benzo[e]pyrrolo[1,2-a][1,4]diazepin-5(11aH)-one); NSC 694501; CAS Registry No. 232931576).

RNA/DNA Antimetabolites: L-alanosine (NSC 153353; CAS Registry No. 59163416); 5-azacytidine (NSC 102816; CAS Registry No. 320672); 5-fluorouracil (NSC 19893; CAS Registry No. 51218); acivicin (NSC 163501; CAS Registry No. 42228922); aminopterin derivative N-[2-chloro-5-[[(2,4-diamino-5-methyl-6-quinazolinyl)methyl]amino]benzoyl-]L-aspartic acid (NSC 132483); aminopterin derivative N-[4-[[(2,4-diamino-5-ethyl-6-quinazolinyl)methyl]amino]benzoyl]L-aspartic acid (NSC 184692); aminopterin derivative N-[2-chloro-4-[[(2,4-diamino-6-pteridinyl)methyl]amino]benzoyl]L-aspartic acid monohydrate (NSC 134033); an antifo ((N^{α}-(4-amino-4-deoxypteroyl)-N⁷-hemiphthaloyl-L-ornithine; NSC 623017)); Baker's soluble antifol (NSC 139105; CAS Registry No. 41191042); dichlorallyl lawsone ((2-(3,3-dichloroallyl)-3-hydroxy-1,4-naphthoquinone; NSC 126771; CAS Registry No. 36417160); brequinar (NSC 368390; CAS Registry No. 96201886); ftorafur ((pro-drug; 5-fluoro-1-(tetrahydro-2-furyl)-uracil; NSC 148958; CAS Registry No. 37076689); 5,6-dihydro-5-azacytidine (NSC 264880; CAS Registry No. 62402317); methotrexate (NSC 740; CAS Registry No. 59052); methotrexate derivative (N-[[4-[[(2,4-diamino-6-pteridinyl)methyl]methylamino]-1-naphthalenyl]carbonyl]L-glutamic acid; NSC 174121); PALA ((N-(phosphonoacetyl)-L-aspartate; NSC 224131; CAS Registry No. 603425565); pyrazofurin (NSC 143095; CAS Registry No. 30868305); trimetrexate (NSC 352122; CAS Registry No. 82952645).

DNA Antimetabolites: 3-HP (NSC 95678; CAS Registry No. 3814797); 2′-deoxy-5-fluorouridine (NSC 27640; CAS Registry No. 50919); 5-HP (NSC 107392; CAS Registry No. 19494894); α-TGDR (α-2′-deoxy-6-thioguanosine; NSC 71851 CAS Registry No. 2133815); aphidicolin glycinate (NSC 303812; CAS Registry No. 92802822); ara C (cytosine arabinoside; NSC 63878; CAS Registry No. 69749); 5-aza-2′-deoxycytidine (NSC 127716; CAS Registry No. 2353335); β-TGDR (β-2′-deoxy-6-thioguanosine; NSC 71261; CAS Registry No. 789617); cyclocytidine (NSC 145668; CAS Registry No. 10212256); guanazole (NSC 1895; CAS Registry No. 1455772); hydroxyurea (NSC 32065; CAS Registry No. 127071); inosine glycodialdehyde (NSC 118994; CAS Registry No. 23590990); macbecin II (NSC 330500; CAS Registry No. 73341738); pyrazoloimidazole (NSC 51143; CAS Registry No. 6714290); thioguanine (NSC 752; CAS Registry No. 154427); thiopurine (NSC 755; CAS Registry No. 50442).

Cell Cycle Modulators: silibinin (CAS Registry No. 22888-70-6); epigallocatechin gallate (EGCG; CAS Registry No. 989515); procyanidin derivatives (e.g., procyanidin A1 [CAS Registry No. 103883030], procyanidin B1 [CAS Registry No. 20315257], procyanidin B4 [CAS Registry No. 29106512], arecatannin B1 [CAS Registry No. 79763283]); isoflavones (e.g., genistein [4′,5,7-trihydroxyisoflavone; CAS Registry No. 446720], daidzein [4′,7-dihydroxyisoflavone, CAS Registry No. 486668]; indole-3-carbinol (CAS Registry No. 700061); quercetin (NSC 9219; CAS Registry No. 117395); estramustine (NSC 89201; CAS Registry No. 2998574); nocodazole (CAS Registry No. 31430189); podophyllotoxin (CAS Registry No. 518285); vinorelbine tartrate (NSC 608210; CAS Registry No. 125317397); cryptophycin (NSC 667642; CAS Registry No. 124689652).

Kinase Inhibitors: afatinib (CAS Registry No. 850140726); axitinib (CAS Registry No. 319460850); ARRY-438162 (binimetinib) (CAS Registry No. 606143899); bosutinib (CAS Registry No. 380843754); cabozantinib (CAS Registry No. 1140909483); ceritinib (CAS Registry No. 1032900256); crizotinib (CAS Registry No. 877399525); dabrafenib (CAS Registry No. 1195765457); dasatinib (NSC 732517; CAS Registry No. 302962498); erlotinib (NSC 718781; CAS Registry No. 183319699); everolimus (NSC 733504; CAS Registry No. 159351696); fostamatinib (NSC 745942; CAS Registry No. 901119355); gefitinib (NSC 715055; CAS Registry No. 184475352); ibrutinib (CAS Registry No. 936563961); imatinib (NSC 716051; CAS Registry No. 220127571); lapatinib (CAS Registry No. 388082788); lenvatinib (CAS Registry No. 857890392); mubritinib (CAS 366017096); nilotinib (CAS Registry No. 923288953); nintedanib (CAS Registry No. 656247175); palbociclib (CAS Registry No. 571190302); pazopanib (NSC 737754; CAS Registry No. 635702646); pegaptanib (CAS Registry No. 222716861); ponatinib (CAS Registry No. 1114544318); rapamycin (NSC 226080; CAS Registry No. 53123889); regorafenib (CAS Registry No. 755037037); AP 23573 (ridaforolimus) (CAS Registry No. 572924540); INCB018424 (ruxolitinib) (CAS Registry No. 1092939177); ARRY-142886 (selumetinib) (NSC 741078; CAS Registry No. 606143-52-6); sirolimus (NSC 226080; CAS Registry No. 53123889); sorafenib (NSC 724772; CAS Registry No. 475207591); sunitinib (NSC 736511; CAS Registry No. 341031547); tofacitinib (CAS Registry No. 477600752); temsirolimus (NSC 683864; CAS Registry No. 163635043); trametinib (CAS Registry No. 871700173); vandetanib (CAS Registry No. 443913733); vemurafenib (CAS Registry No. 918504651); SU6656 (CAS Registry No. 330161870); CEP-701 (lesaurtinib) (CAS Registry No. 111358884); XL019 (CAS Registry No. 945755566); PD-325901 (CAS Registry No. 391210109); PD-98059 (CAS Registry No. 167869218); ATP-competitive TORC1/TORC2 inhibitors including PI-103 (CAS Registry No. 371935749), PP242 (CAS Registry No. 1092351671), PP30 (CAS Registry No. 1092788094), Torin 1 (CAS Registry No. 1222998368), LY294002 (CAS Registry No. 154447366), XL-147 (CAS Registry No. 934526893), CAL-120 (CAS Registry No. 870281348), ETP-45658 (CAS Registry No. 1198357797), PX 866 (CAS Registry No. 502632668), GDC-0941 (CAS Registry No. 957054307), BGT226 (CAS Registry No. 1245537681), BEZ235 (CAS Registry No. 915019657), XL-765 (CAS Registry No. 934493762).

Protein Synthesis Inhibitors: acriflavine (CAS Registry No. 65589700); amikacin (NSC 177001; CAS Registry No. 39831555); arbekacin (CAS Registry No. 51025855); astromicin (CAS Registry No. 55779061); azithromycin (NSC 643732; CAS Registry No. 83905015); bekanamycin (CAS Registry No. 4696768); chlortetracycline (NSC 13252; CAS Registry No. 64722); clarithromycin (NSC 643733; CAS Registry No. 81103119); clindamycin (CAS Registry No. 18323449); clomocycline (CAS Registry No. 1181540); cycloheximide (CAS Registry No. 66819); dactinomycin (NSC 3053; CAS Registry No. 50760); dalfopristin (CAS Registry No. 112362502); demeclocycline (CAS Registry No. 127333); dibekacin (CAS Registry No. 34493986); dihydrostreptomycin (CAS Registry No. 128461); dirithromycin (CAS Registry No. 62013041); doxycycline (CAS Registry No. 17086281); emetine (NSC 33669; CAS Registry No. 483181); erythromycin (NSC 55929; CAS Registry No. 114078); flurithromycin (CAS Registry No. 83664208); framycetin (neomycin B; CAS Registry No. 119040); gentamycin (NSC 82261; CAS Registry No. 1403663); glycylcyclines, such as tigecycline (CAS Registry No. 220620097); hygromycin B (CAS Registry No. 31282049); isepamicin (CAS Registry No. 67814760); josamycin (NSC 122223; CAS Registry No. 16846245); kanamycin (CAS Registry No. 8063078); ketolides such as telithromycin (CAS Registry No. 191114484), cethromycin (CAS Registry No. 205110481), and solithromycin (CAS Registry No. 760981837); lincomycin (CAS Registry No. 154212); lymecycline (CAS Registry No. 992212); meclocycline (NSC 78502; CAS Registry No. 2013583); metacycline (rondomycin; NSC 356463; CAS Registry No. 914001); midecamycin (CAS Registry No. 35457808); minocycline (NSC 141993; CAS Registry No. 10118908); miocamycin (CAS Registry No. 55881077); neomycin (CAS Registry No. 119040); netilmicin (CAS Registry No. 56391561); oleandomycin (CAS Registry No. 3922905); oxazolidinones, such as eperezolid (CAS Registry No. 165800044), linezolid (CAS Registry No. 165800033), posizolid (CAS Registry No. 252260029), radezolid (CAS Registry No. 869884786), ranbezolid (CAS Registry No. 392659380), sutezolid (CAS Registry No. 168828588), tedizolid (CAS Registry No. 856867555); oxytetracycline (NSC 9169; CAS Registry No. 2058460); paromomycin (CAS Registry No. 7542372); penimepicycline (CAS Registry No. 4599604); peptidyl transferase inhibitors, e.g., chloramphenicol (NSC 3069; CAS Registry No. 56757) and derivatives such as azidamfenicol (CAS Registry No. 13838089), florfenicol (CAS Registry No. 73231342), and thiamphenicol (CAS Registry No. 15318453), and pleuromutilins such as retapamulin (CAS Registry No. 224452668), tiamulin (CAS Registry No. 55297955), valnemulin (CAS Registry No. 101312929); pirlimycin (CAS Registry No. 79548735); puromycin (NSC 3055; CAS Registry No. 53792); quinupristin (CAS Registry No. 120138503); ribostamycin (CAS Registry No. 53797356); rokitamycin (CAS Registry No. 74014510); rolitetracycline (CAS Registry No. 751973); roxithromycin (CAS Registry No. 80214831); sisomicin (CAS Registry No. 32385118); spectinomycin (CAS Registry No. 1695778); spiramycin (CAS Registry No. 8025818); streptogramins such as pristinamycin (CAS Registry No. 270076603), quinupristin/dalfopristin (CAS Registry No. 126602899), and virginiamycin (CAS Registry No. 11006761); streptomycin (CAS Registry No. 57921); tetracycline (NSC 108579; CAS Registry No. 60548); tobramycin (CAS Registry No. 32986564); troleandomycin (CAS Registry No. 2751099); tylosin (CAS Registry No. 1401690); verdamicin (CAS Registry No. 49863481).

Histone Deacetylase Inhibitors: abexinostat (CAS Registry No. 783355602); belinostat (NSC 726630; CAS Registry No. 414864009); chidamide (CAS Registry No. 743420022); entinostat (CAS Registry No. 209783802); givinostat (CAS Registry No. 732302997); mocetinostat (CAS Registry No. 726169739); panobinostat (CAS Registry No. 404950807); quisinostat (CAS Registry No. 875320299); resminostat (CAS Registry No. 864814880); romidepsin (CAS Registry No. 128517077); sulforaphane (CAS Registry No. 4478937); thioureidobutyronitrile (Kevetrin™; CAS Registry No. 6659890); valproic acid (NSC 93819; CAS Registry No. 99661); vorinostat (NSC 701852; CAS Registry No. 149647789); ACY-1215 (rocilinostat; CAS Registry No. 1316214524); CUDC-101 (CAS Registry No. 1012054599); CHR-2845 (tefinostat; CAS Registry No. 914382608); CHR-3996 (CAS Registry No. 1235859138); 4SC-202 (CAS Registry No. 910462430); CG200745 (CAS Registry No. 936221339); SB939 (pracinostat; CAS Registry No. 929016966).

Mitochondria Inhibitors: pancratistatin (NSC 349156; CAS Registry No. 96281311); rhodamine-123 (CAS Registry No. 63669709); edelfosine (NSC 324368; CAS Registry No. 70641519); d-alpha-tocopherol succinate (NSC 173849; CAS Registry No. 4345033); compound 11β (CAS Registry No. 865070377); aspirin (NSC 406186; CAS Registry No. 50782); ellipticine (CAS Registry No. 519233); berberine (CAS Registry No. 633658); cerulenin (CAS Registry No. 17397896); GX015-070 (Obatoclax®; 1H-Indole, 2-(2-((3,5-dimethyl-1H-pyrrol-2-yl)methylene)-3-methoxy-2H-pyrrol-5-yl)-; NSC 729280; CAS Registry No. 803712676); celastrol (tripterine; CAS Registry No. 34157830); metformin (NSC 91485; CAS Registry No. 1115704); Brilliant green (NSC 5011; CAS Registry No. 633034); ME-344 (CAS Registry No. 1374524556).

Antimitotic Agents: allocolchicine (NSC 406042); auristatins, such as MMAE (monomethyl auristatin E; CAS Registry No. 474645-27-7) and MMAF (monomethyl auristatin F; CAS Registry No. 745017-94-1; halichondrin B (NSC 609395); colchicine (NSC 757; CAS Registry No. 64868); cholchicine derivative (N-benzoyl-deacetyl benzamide; NSC 33410; CAS Registry No. 63989753); dolastatin 10 (NSC 376128; CAS Registry No 110417-88-4); maytansine (NSC 153858; CAS Registry No. 35846-53-8); rhozoxin (NSC 332598; CAS Registry No. 90996546); taxol (NSC 125973; CAS Registry No. 33069624); taxol derivative ((2′-N-[3-(dimethylamino)propyl]glutaramate taxol; NSC 608832); thiocolchicine (3-demethylthiocolchicine; NSC 361792); trityl cysteine (NSC 49842; CAS Registry No. 2799077); vinblastine sulfate (NSC 49842; CAS Registry No. 143679); vincristine sulfate (NSC 67574; CAS Registry No. 2068782).

Any of these agents that include or that can be modified to include a site of attachment to a MBM can be included in the ADCs disclosed herein.

In a specific embodiment, the cytotoxic and/or cytostatic agent is an antimitotic agent.

In another specific embodiment, the cytotoxic and/or cytostatic agent is an auristatin, for example, monomethyl auristatin E (“MMAE”) or monomethyl auristatin F (“MMAF”).

7.13.2. ADC Linkers

In the ADCs, the cytotoxic and/or cytostatic agents are linked to the MBM by way of ADC linkers. The ADC linker linking a cytotoxic and/or cytostatic agent to the MBM of an ADC can be short, long, hydrophobic, hydrophilic, flexible or rigid, or can be composed of segments that each independently have one or more of the above-mentioned properties such that the linker can include segments having different properties. The linkers can be polyvalent such that they covalently link more than one agent to a single site on the MBM, or monovalent such that covalently they link a single agent to a single site on the MBM.

As will be appreciated by skilled artisans, the ADC linkers link cytotoxic and/or cytostatic agents to the MBM by forming a covalent linkage to the cytotoxic and/or cytostatic agent at one location and a covalent linkage to the MBM at another. The covalent linkages are formed by reaction between functional groups on the ADC linker and functional groups on the agents and MBM. As used herein, the expression “ADC linker” is intended to include (i) unconjugated forms of the ADC linker that include a functional group capable of covalently linking the ADC linker to a cytotoxic and/or cytostatic agent and a functional group capable of covalently linking the ADC linker to a MBM; (ii) partially conjugated forms of the ADC linker that include a functional group capable of covalently linking the ADC linker to a MBM and that is covalently linked to a cytotoxic and/or cytostatic agent, or vice versa; and (iii) fully conjugated forms of the ADC linker that are covalently linked to both a cytotoxic and/or cytostatic agent and a MBM. In some specific embodiments of ADC linkers and ADCs, as well as synthons used to conjugate linker-agents to MBMs, moieties comprising the functional groups on the ADC linker and covalent linkages formed between the ADC linker and MBM are specifically illustrated as Rₓ and XY, respectively.

The ADC linkers are preferably, but need not be, chemically stable to conditions outside the cell, and can be designed to cleave, immolate and/or otherwise specifically degrade inside the cell. Alternatively, ADC linkers that are not designed to specifically cleave or degrade inside the cell can be used. Choice of stable versus unstable ADC linker can depend upon the toxicity of the cytotoxic and/or cytostatic agent. For agents that are toxic to normal cells, stable linkers are preferred. Agents that are selective or targeted and have lower toxicity to normal cells can utilize, chemical stability of the ADC linker to the extracellular milieu is less important. A wide variety of ADC linkers useful for linking drugs to MBMs in the context of ADCs are known in the art. Any of these ADC linkers, as well as other ADC linkers, can be used to link the cytotoxic and/or cytostatic agents to the MBM of the ADCs of the disclosure.

Exemplary polyvalent ADC linkers that can be used to link many cytotoxic and/or cytostatic agents to a single MBM molecule are described, for example, in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640. For example, the Fleximer linker technology developed by Mersana et al. has the potential to enable high-DAR ADCs with good physicochemical properties. As shown below, the Mersana technology is based on incorporating drug molecules into a solubilizing poly-acetal backbone via a sequence of ester bonds. The methodology renders highly loaded ADCs (DAR up to 20) while maintaining good physicochemical properties.

Additional examples of dendritic type linkers can be found in US 2006/116422; US 2005/271615; de Groot et al., 2003, Angew. Chem. Int. Ed. 42:4490-4494; Amir et al., 2003, Angew. Chem. Int. Ed. 42:4494-4499; Shamis et al., 2004, J. Am. Chem. Soc. 126:1726-1731; Sun et al., 2002, Bioorganic & Medicinal Chemistry Letters 12:2213-2215; Sun et al., 2003, Bioorganic & Medicinal Chemistry 11:1761-1768; King et al., 2002, Tetrahedron Letters 43:1987-1990.

Exemplary monovalent ADC linkers that can be used are described, for example, in Nolting, 2013, Antibody-Drug Conjugates, Methods in Molecular Biology 1045:71-100; Kitson et al., 2013, CROs-MOs-Chemica-ggi—Chemistry Today 31(4):30-38; Ducry et al., 2010, Bioconjugate Chem. 21:5-13; Zhao et al., 2011, J. Med. Chem. 54:3606-3623; U.S. Pat. Nos. 7,223,837; 8,568,728; 8,535,678; and WO2004010957.

By way of example and not limitation, some cleavable and noncleavable ADC linkers that can be included in the ADCs are described below.

7.13.2.1. Cleavable ADC Linkers

In certain embodiments, the ADC linker selected is cleavable in vivo. Cleavable ADC linkers can include chemically or enzymatically unstable or degradable linkages. Cleavable ADC linkers generally rely on processes inside the cell to liberate the drug, such as reduction in the cytoplasm, exposure to acidic conditions in the lysosome, or cleavage by specific proteases or other enzymes within the cell. Cleavable ADC linkers generally incorporate one or more chemical bonds that are either chemically or enzymatically cleavable while the remainder of the ADC linker is noncleavable. In certain embodiments, an ADC linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing ADC linkers are the acidic environment of endosomes and lysosomes, while the disulfide containing ADC linkers are reduced in the cytosol, which contains high thiol concentrations, e.g., glutathione. In certain embodiments, the plasma stability of an ADC linker comprising a chemically labile group can be increased by introducing steric hindrance using substituents near the chemically labile group.

Acid-labile groups, such as hydrazone, remain intact during systemic circulation in the blood's neutral pH environment (pH 7.3-7.5), undergo hydrolysis, and release the drug once the ADC is internalized into mildly acidic endosomal (pH 5.0-6.5) and lysosomal (pH 4.5-5.0) compartments of the cell. This pH dependent release mechanism has been associated with nonspecific release of the drug. To increase the stability of the hydrazone group of the ADC linker, the ADC linker can be varied by chemical modification, e.g., substitution, allowing tuning to achieve more efficient release in the lysosome with a minimized loss in circulation.

Hydrazone-containing ADC linkers can contain additional cleavage sites, such as additional acid-labile cleavage sites and/or enzymatically labile cleavage sites. ADCs including exemplary hydrazone-containing ADC linkers include the following structures:

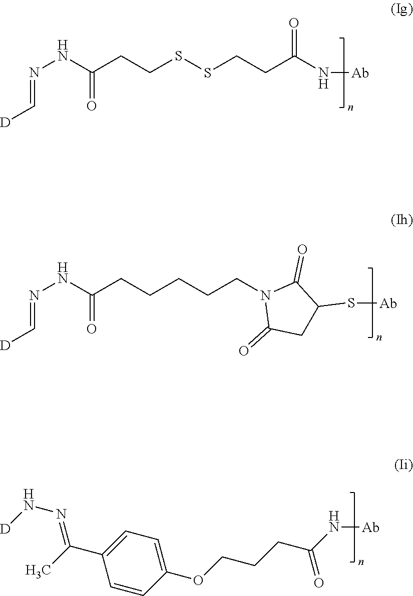

wherein D and Ab represent the cytotoxic and/or cytostatic agent (drug) and Ab, respectively, and n represents the number of drug-ADC linkers linked to the MBM. In certain ADC linkers such as linker (Ig), the ADC linker comprises two cleavable groups—a disulfide and a hydrazone moiety. For such ADC linkers, effective release of the unmodified free drug requires acidic pH or disulfide reduction and acidic pH. Linkers such as (1h) and (Ii) have been shown to be effective with a single hydrazone cleavage site.

Additional ADC linkers which remain intact during systemic circulation and undergo hydrolysis and release the drug when the ADC is internalized into acidic cellular compartments include carbonates. Such ADC linkers can be useful in cases where the cytotoxic and/or cytostatic agent can be covalently attached through an oxygen.

Other acid-labile groups that can be included in ADC linkers include cis-aconityl-containing ADC linkers. cis-Aconityl chemistry uses a carboxylic acid juxtaposed to an amide bond to accelerate amide hydrolysis under acidic conditions.

Cleavable ADC linkers can also include a disulfide group. Disulfides are thermodynamically stable at physiological pH and are designed to release the drug upon internalization inside cells, wherein the cytosol provides a significantly more reducing environment compared to the extracellular environment. Scission of disulfide bonds generally requires the presence of a cytoplasmic thiol cofactor, such as (reduced) glutathione (GSH), such that disulfide-containing ADC linkers are reasonably stable in circulation, selectively releasing the drug in the cytosol. The intracellular enzyme protein disulfide isomerase, or similar enzymes capable of cleaving disulfide bonds, can also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol in circulation. Where irregular blood flow leads to a hypoxic state, this results in enhanced activity of reductive enzymes and therefore even higher glutathione concentrations. In certain embodiments, the in vivo stability of a disulfide-containing ADC linker can be enhanced by chemical modification of the ADC linker, e.g., use of steric hindrance adjacent to the disulfide bond.

ADCs including exemplary disulfide-containing ADC linkers include the following structures:

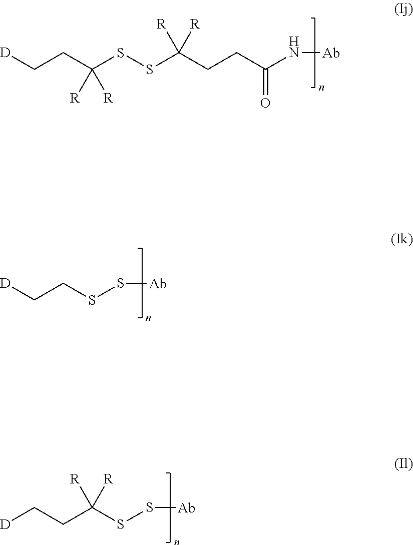

wherein D and Ab represent the drug and MBM, respectively, n represents the number of drug-ADC linkers linked to the MBM and R is independently selected at each occurrence from hydrogen or alkyl, for example. In certain embodiments, increasing steric hindrance adjacent to the disulfide bond increases the stability of the ADC linker. Structures such as (Ij) and (II) show increased in vivo stability when one or more R groups is selected from a lower alkyl such as methyl.

Another type of cleavable ADC linker that can be used is an ADC linker that is specifically cleaved by an enzyme. Such ADC linkers are typically peptide-based or include peptidic regions that act as substrates for enzymes. Peptide based ADC linkers tend to be more stable in plasma and extracellular milieu than chemically labile ADC linkers. Peptide bonds generally have good serum stability, as lysosomal proteolytic enzymes have very low activity in blood due to endogenous inhibitors and the unfavorably high pH value of blood compared to lysosomes. Release of a drug from a MBM occurs specifically due to the action of lysosomal proteases, e.g., cathepsin and plasmin. These proteases can be present at elevated levels in certain tumor cells.

In exemplary embodiments, the cleavable peptide is selected from tetrapeptides such as Gly-Phe-Leu-Gly (SEQ ID NO:131), Ala-Leu-Ala-Leu (SEQ ID NO:132) or dipeptides such as Val-Cit, Val-Ala, Met-(D)Lys, Asn-(D)Lys, Val-(D)Asp, Phe-Lys, Ile-Val, Asp-Val, His-Val, NorVal-(D)Asp, Ala-(D)Asp 5, Met-Lys, Asn-Lys, Ile-Pro, Me3Lys-Pro, PhenylGly-(D)Lys, Met-(D)Lys, Asn-(D)Lys, Pro-(D)Lys, Met-(D)Lys, Asn-(D)Lys, AM Met-(D)Lys, Asn-(D)Lys, AW Met-(D)Lys, and Asn-(D)Lys. In certain embodiments, dipeptides are preferred over longer polypeptides due to hydrophobicity of the longer peptides.

A variety of dipeptide-based cleavable ADC linkers useful for linking drugs such as doxorubicin, mitomycin, camptothecin, pyrrolobenzodiazepine, tallysomycin and auristatin/auristatin family members to MBMs have been described (see, Dubowchik et al., 1998, J. Org. Chem. 67:1866-1872; Dubowchik et al., 1998, Bioorg. Med. Chem. Lett. 8(21):3341-3346; Walker et al., 2002, Bioorg. Med. Chem. Lett. 12:217-219; Walker et al., 2004, Bioorg. Med. Chem. Lett. 14:4323-4327; Sutherland et al., 2013, Blood 122: 1455-1463; and Francisco et al., 2003, Blood 102:1458-1465). All of these dipeptide ADC linkers, or modified versions of these dipeptide ADC linkers, can be used in the ADCs of the disclosure. Other dipeptide ADC linkers that can be used include those found in ADCs such as Seattle Genetics' Brentuximab Vendotin SGN-35 (Adcetris™), Seattle Genetics SGN-75 (anti-CD-70, Val-Cit-monomethyl auristatin F(MMAF), Seattle Genetics SGN-CD33A (anti-CD-33, Val-Ala-(SGD-1882)), Celldex Therapeutics glembatumumab (CDX-011) (anti-NMB, Val-Cit-monomethyl auristatin E (MMAE), and Cytogen PSMA-ADC (PSMA-ADC-1301) (anti-PSMA, Val-Cit-MMAE).

Enzymatically cleavable ADC linkers can include a self-immolative spacer to spatially separate the drug from the site of enzymatic cleavage. The direct attachment of a drug to a peptide ADC linker can result in proteolytic release of an amino acid adduct of the drug, thereby impairing its activity. The use of a self-immolative spacer allows for the elimination of the fully active, chemically unmodified drug upon amide bond hydrolysis.

One self-immolative spacer is the bifunctional para-aminobenzyl alcohol group, which is linked to the peptide through the amino group, forming an amide bond, while amine containing drugs can be attached through carbamate functionalities to the benzylic hydroxyl group of the ADC linker (PABC). The resulting prodrugs are activated upon protease-mediated cleavage, leading to a 1,6-elimination reaction releasing the unmodified drug, carbon dioxide, and remnants of the ADC linker group. The following scheme depicts the fragmentation of p-amidobenzyl ether and release of the drug:

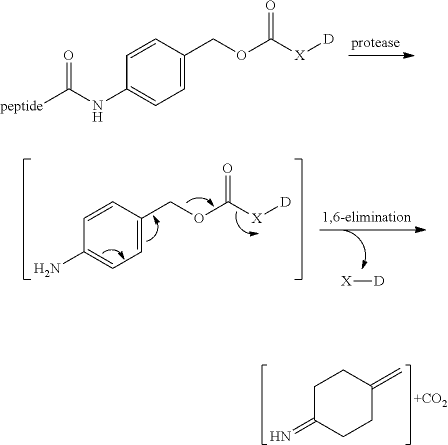

wherein X-D represents the unmodified drug.

Heterocyclic variants of this self-immolative group have also been described. See for example, U.S. Pat. No. 7,989,434.

In some embodiments, the enzymatically cleavable ADC linker is a β-glucuronic acid-based ADC linker. Facile release of the drug can be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based ADC linkers can be used to circumvent the tendency of an ADC to undergo aggregation due to the hydrophilic nature of β-glucuronides. In some embodiments, β-glucuronic acid-based ADC linkers are preferred as ADC linkers for ADCs linked to hydrophobic drugs. The following scheme depicts the release of the drug from and ADC containing a β-glucuronic acid-based ADC linker:

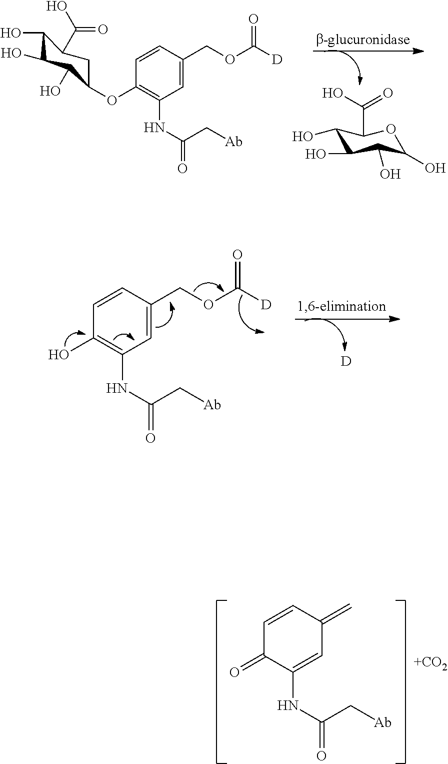

A variety of cleavable β-glucuronic acid-based ADC linkers useful for linking drugs such as auristatins, camptothecin and doxorubicin analogues, CBI minor-groove binders, and psymberin to MBMs have been described (see, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013; Jeffrey et al., 2006, Bioconjug. Chem. 17:831-840; Jeffrey et al., 2007, Bioorg. Med. Chem. Lett. 17:2278-2280; and Jiang et al., 2005, J. Am. Chem. Soc. 127:11254-11255). All of these β-glucuronic acid-based ADC linkers can be used in the ADCs of the disclosure.

Additionally, cytotoxic and/or cytostatic agents containing a phenol group can be covalently bonded to an ADC linker through the phenolic oxygen. One such ADC linker, described in WO 2007/089149, relies on a methodology in which a diamino-ethane “SpaceLink” is used in conjunction with traditional “PABO”-based self-immolative groups to deliver phenols. The cleavage of the ADC linker is depicted schematically below, where D represents a cytotoxic and/or cytostatic agent having a phenolic hydroxyl group.

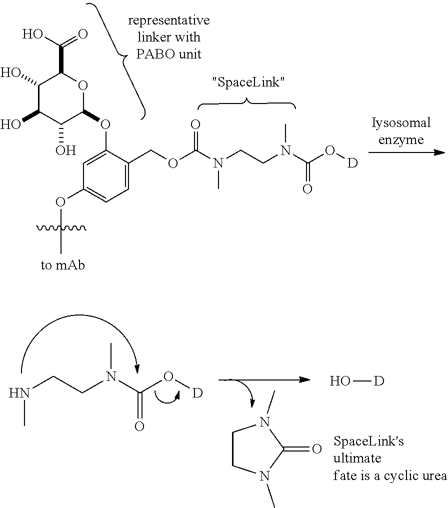

Cleavable ADC linkers can include noncleavable portions or segments, and/or cleavable segments or portions can be included in an otherwise non-cleavable ADC linker to render it cleavable. By way of example only, polyethylene glycol (PEG) and related polymers can include cleavable groups in the polymer backbone. For example, a polyethylene glycol or polymer ADC linker can include one or more cleavable groups such as a disulfide, a hydrazone or a dipeptide.

Other degradable linkages that can be included in ADC linkers include ester linkages formed by the reaction of PEG carboxylic acids or activated PEG carboxylic acids with alcohol groups on a biologically active agent, wherein such ester groups generally hydrolyze under physiological conditions to release the biologically active agent. Hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In certain embodiments, the ADC linker comprises an enzymatically cleavable peptide moiety, for example, an ADC linker comprising structural formula (IVa) or (IVb):

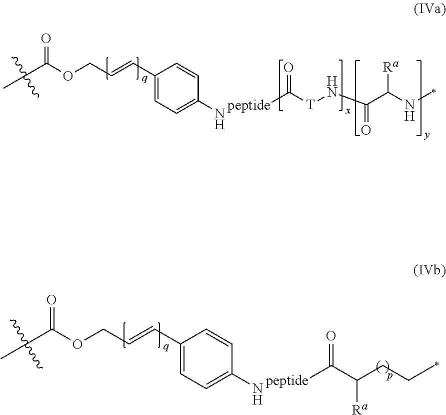

or a salt thereof, wherein: peptide represents a peptide (illustrated C→N and not showing the carboxy and amino “termini”) cleavable by a lysosomal enzyme; T represents a polymer comprising one or more ethylene glycol units or an alkylene chain, or combinations thereof; R^{a} is selected from hydrogen, alkyl, sulfonate and methyl sulfonate; p is an integer ranging from 0 to 5; q is 0 or 1; x is 0 or 1; y is 0 or 1; [symbol] represents the point of attachment of the ADC linker to a cytotoxic and/or cytostatic agent; and * represents the point of attachment to the remainder of the ADC linker.

In certain embodiments, the peptide is selected from a tripeptide or a dipeptide. In particular embodiments, the dipeptide is selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Val-Lys; Ala-Lys; Phe-Cit; Leu-Cit; Ile-Cit; Phe-Arg; and Trp-Cit. In certain embodiments, the dipeptide is selected from: Cit-Val; and Ala-Val.

Specific exemplary embodiments of ADC linkers according to structural formula (IVa) that can be included in the ADCs include the ADC linkers illustrated below (as illustrated, the ADC linkers include a group suitable for covalently linking the ADC linker to a MBM):

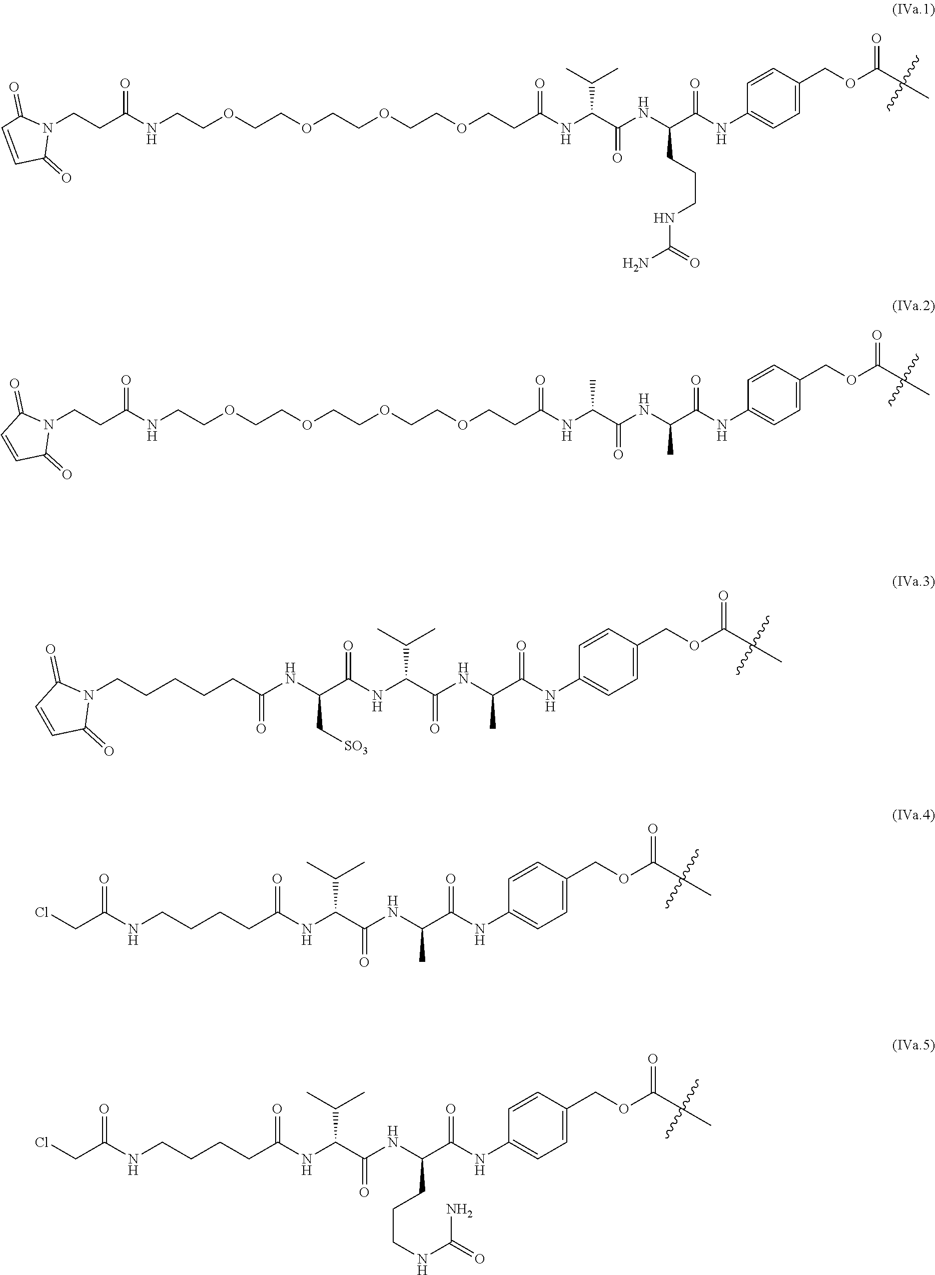

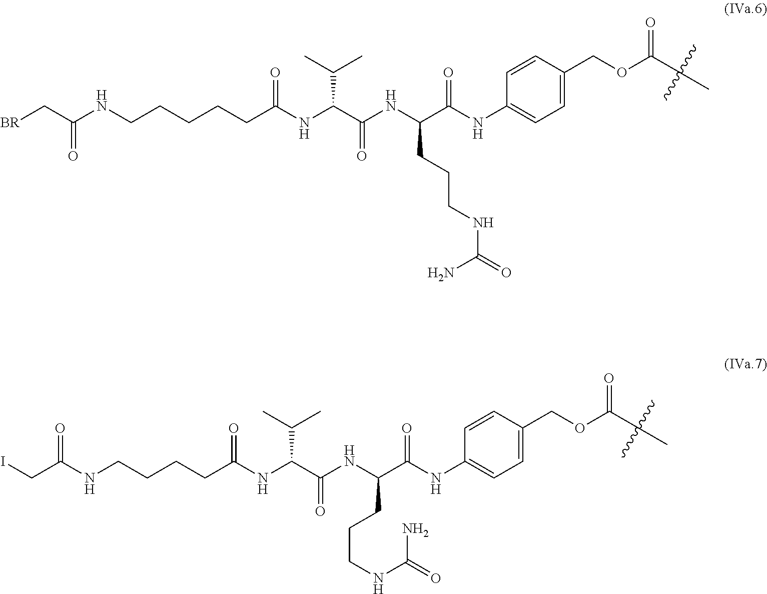

Specific exemplary embodiments of ADC linkers according to structural formula (IVb) that can be included in the ADCs include the ADC linkers illustrated below (as illustrated, the ADC linkers include a group suitable for covalently linking the ADC linker to a MBM):

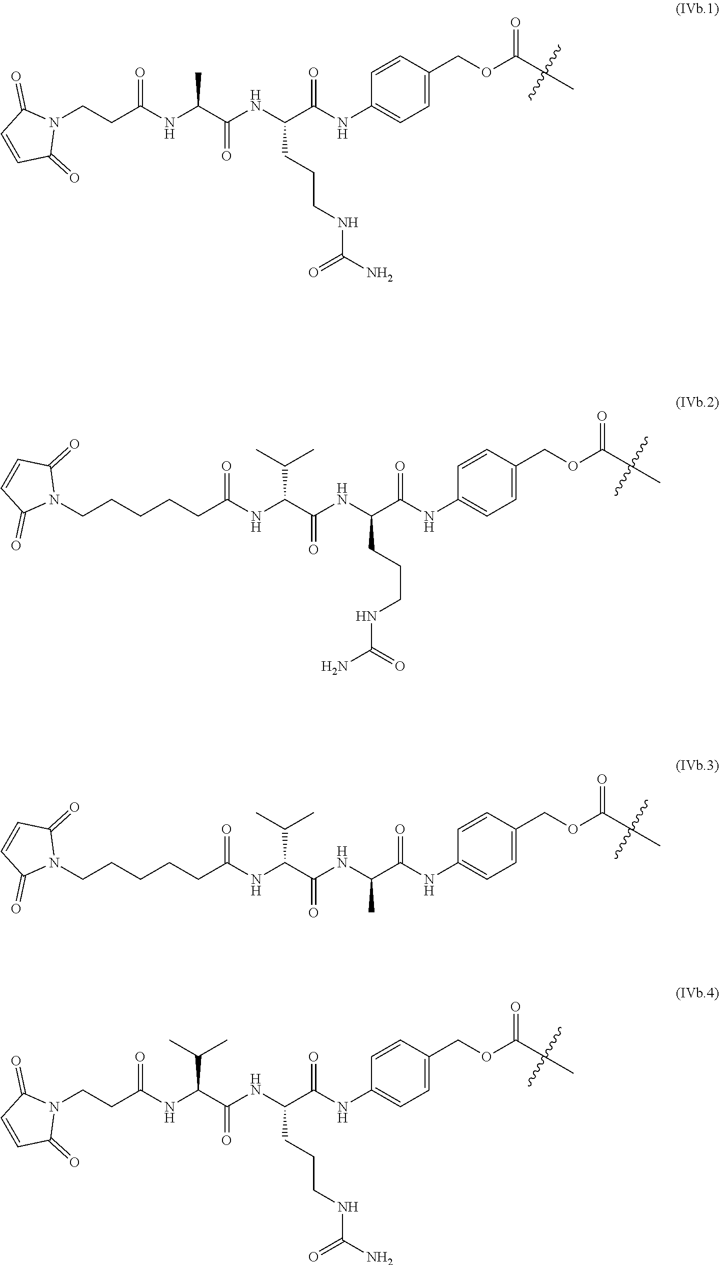 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@@H](CCCNC(N)=O)NC(=O)[C@H](NC(=O)CCCCCN2C(=O)C=CC2=O)C(C)C)cc1 ; *C(=O)OCc1ccc(NC(=O)[C@@H](C)NC(=O)[C@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1]

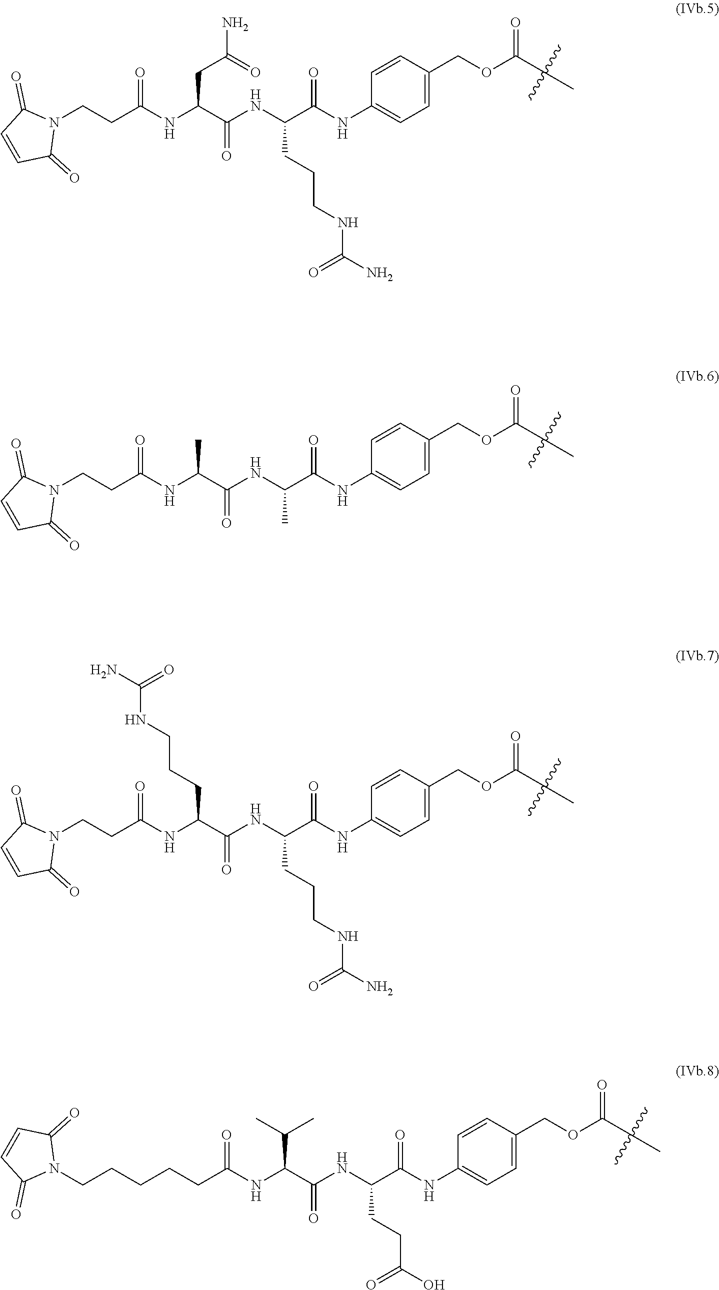 [SMILES: *C(=O)OCc1ccc(NC(=O)[C@H](CCCNC(N)=O)NC(=O)[C@H](CC(N)=O)NC(=O)CCN2C(=O)C=CC2=O)cc1 ; *C(=O)OCc1ccc(NC(=O)[C@H](C)NC(=O)[C@H](C)NC(=O)CCN2C(=O)C=CC2=O)cc1 ; *C(=O)OCc1ccc(NC(=O)[C@H](CCCNC(N)=O)NC(=O)[C@H](CCCNC(N)=O)NC(=O)CCN2C(=O)C=CC2=O)cc1 ; *C(=O)OCc1ccc(NC(=O)[C@H](CCC(=O)O)NC(=O)[C@@H](NC(=O)CCCCCN2C(=O)C=CC2=O)C(C)C)cc1]

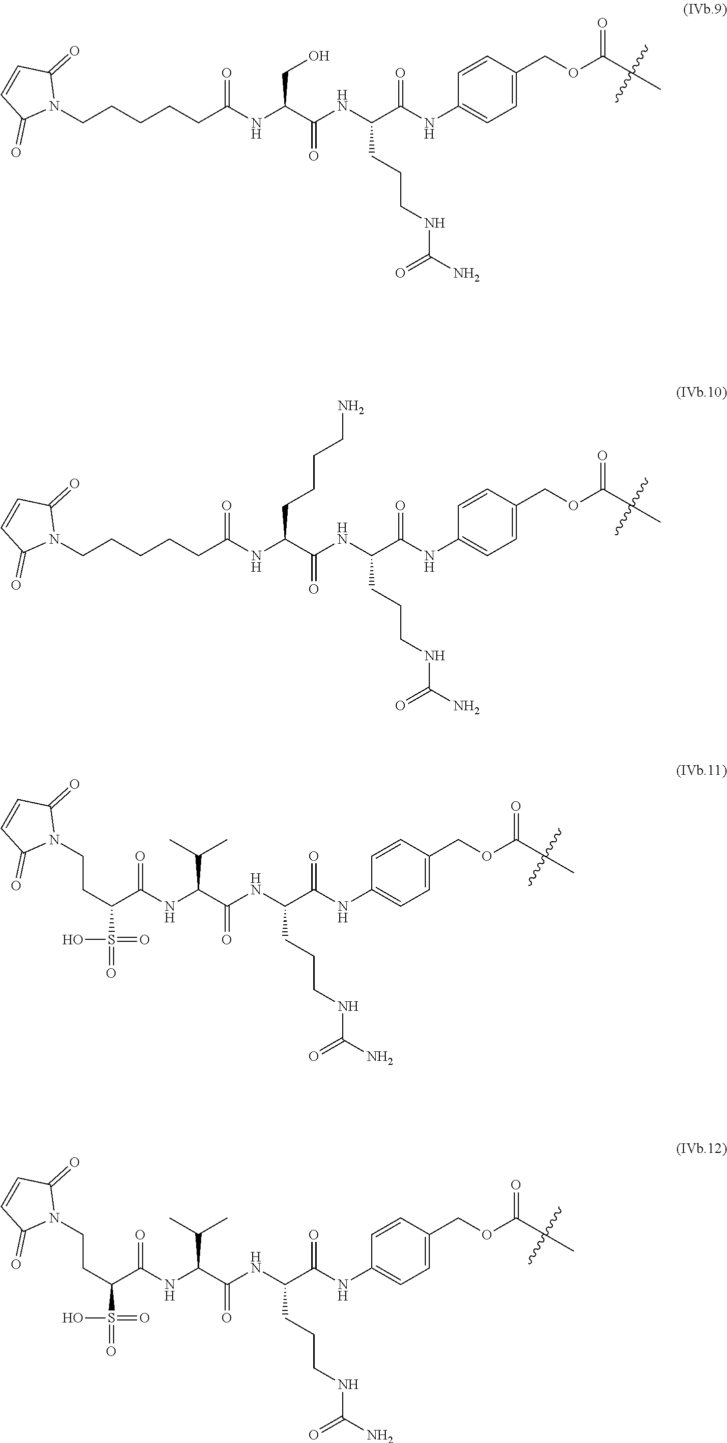 [SMILES: *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1]

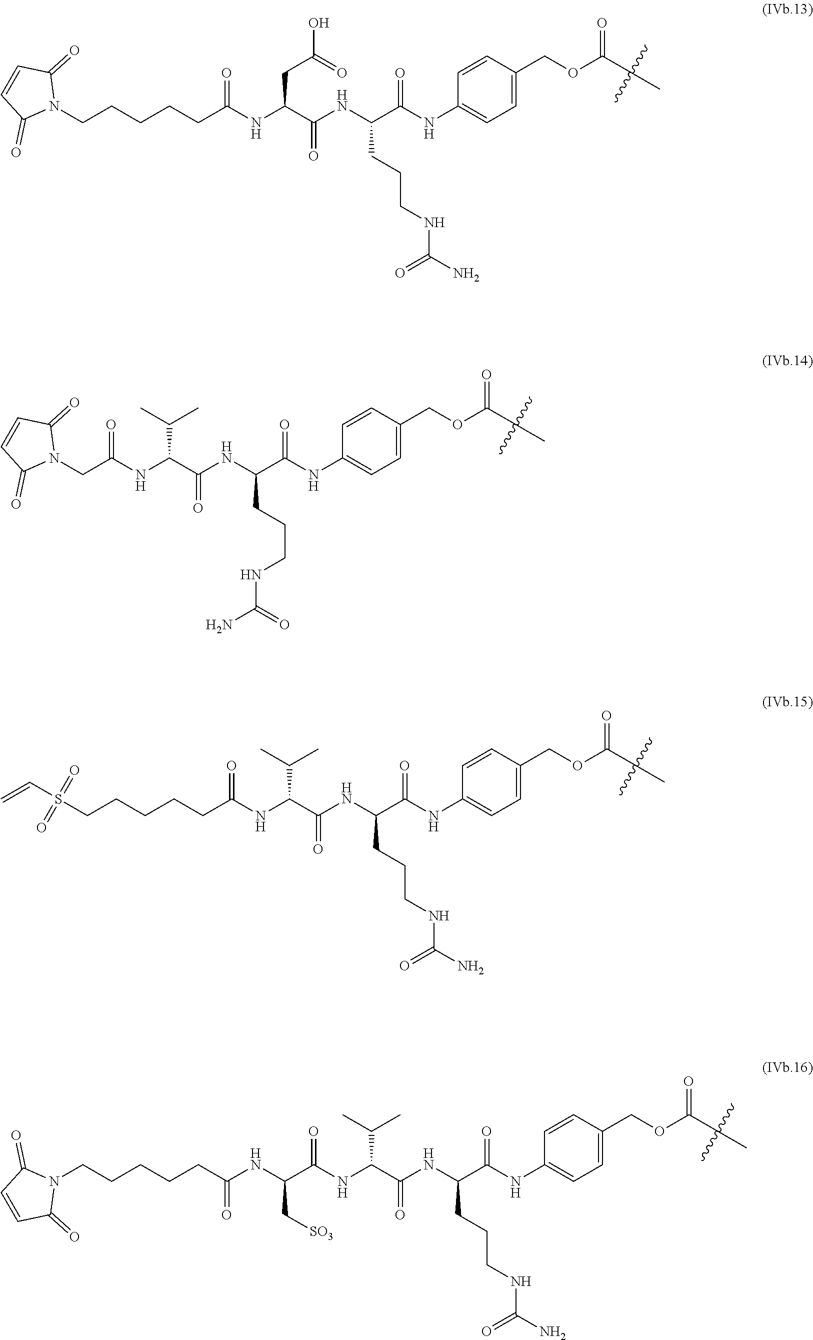

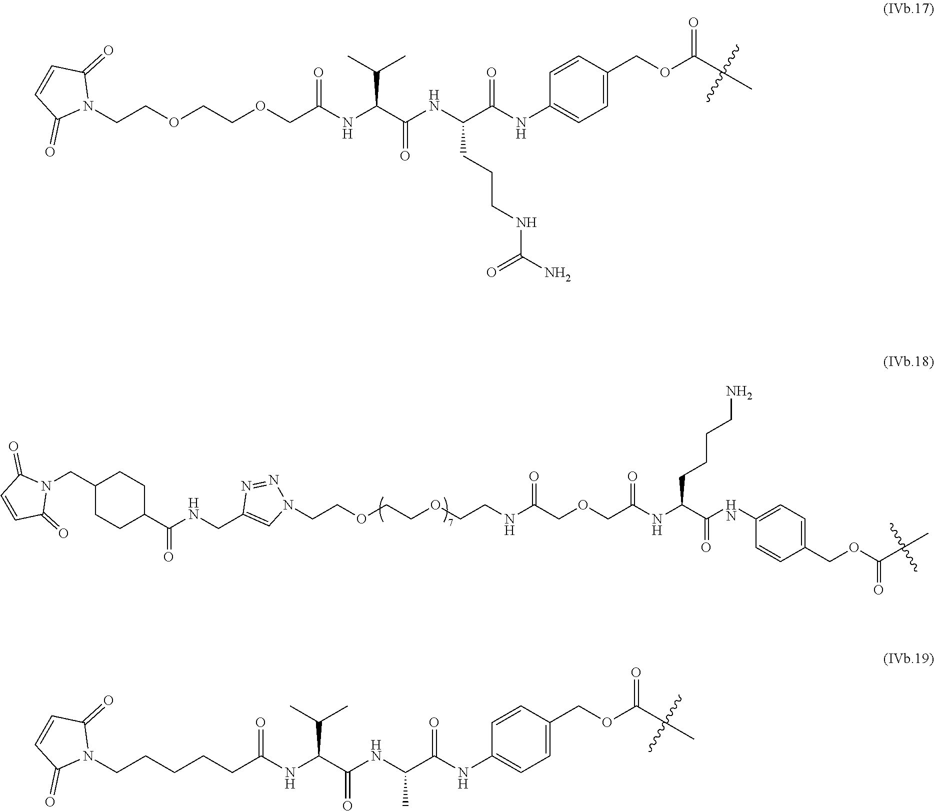

In certain embodiments, the ADC linker comprises an enzymatically cleavable peptide moiety, for example, an ADC linker comprising structural formula (IVc) or (IVd):

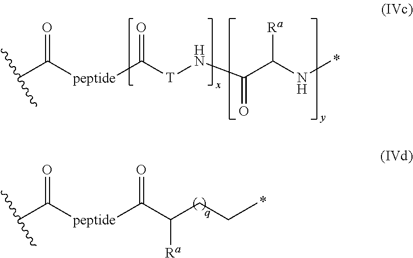

or a salt thereof, wherein: peptide represents a peptide (illustrated C→N and not showing the carboxy and amino “termini”) cleavable by a lysosomal enzyme; T represents a polymer comprising one or more ethylene glycol units or an alkylene chain, or combinations thereof; R^{a} is selected from hydrogen, alkyl, sulfonate and methyl sulfonate; p is an integer ranging from 0 to 5; q is 0 or 1; x is 0 or 1; y is 0 or 1; .x [symbol] represents the point of attachment of the ADC linker to a cytotoxic and/or cytostatic agent; and * represents the point of attachment to the remainder of the ADC linker.

Specific exemplary embodiments of ADC linkers according to structural formula (IVc) that can be included in the ADCs include the ADC linkers illustrated below (as illustrated, the ADC linkers include a group suitable for covalently linking the ADC linker to a MBM):

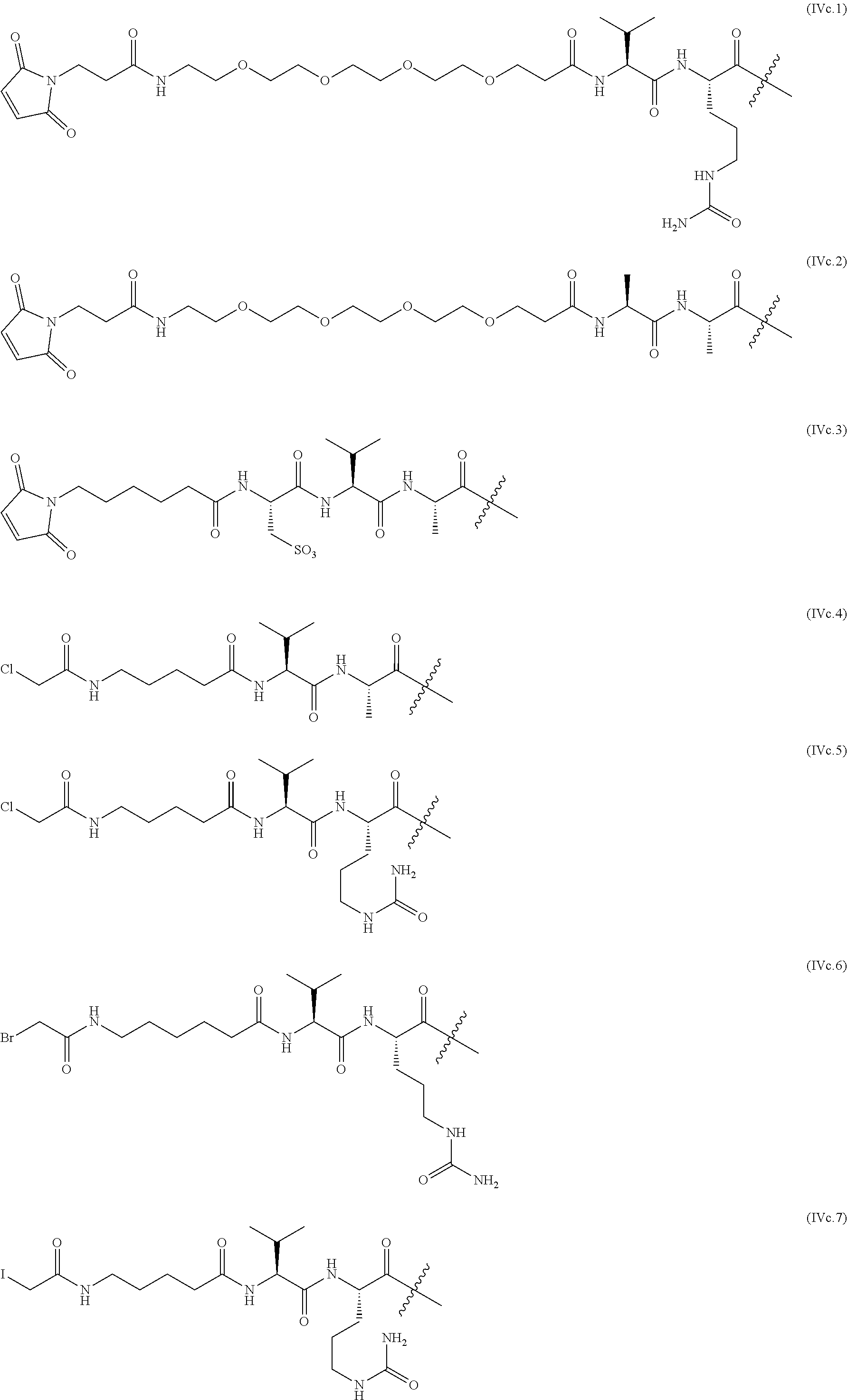

Specific exemplary embodiments of ADC linkers according to structural formula (IVd) that can be included in the ADCs include the ADC linkers illustrated below (as illustrated, the ADC linkers include a group suitable for covalently linking the ADC linker to a MBM):

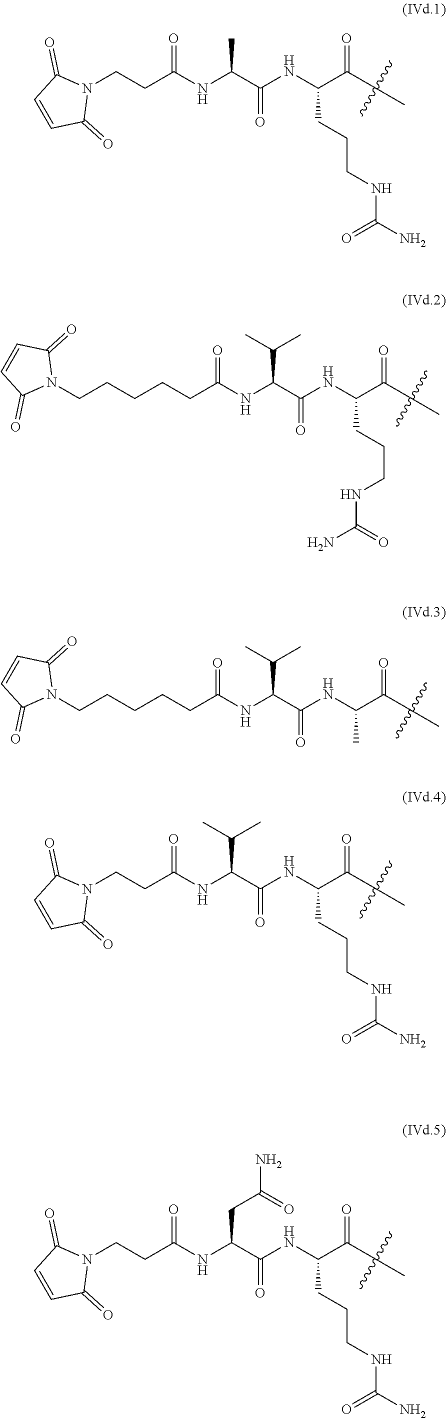 [SMILES: *C(=O)[C@H](CCCNC(N)=O)NC(=O)[C@H](C)NC(=O)CCN1C(=O)C=CC1=O ; *C(=O)[C@H](CCCNC(N)=O)NC(=O)[C@@H](NC(=O)CCCCCN1C(=O)C=CC1=O)C(C)C ; *C(=O)[C@H](C)NC(=O)[C@@H](NC(=O)CCCCCN1C(=O)C=CC1=O)C(C)C ; *C(=O)[C@H](CCCNC(N)=O)NC(=O)[C@@H](NC(=O)CCN1C(=O)C=CC1=O)C(C)C ; *C(=O)[C@H](CCCNC(N)=O)NC(=O)[C@H](CC(N)=O)NC(=O)CCN1C(=O)C=CC1=O]

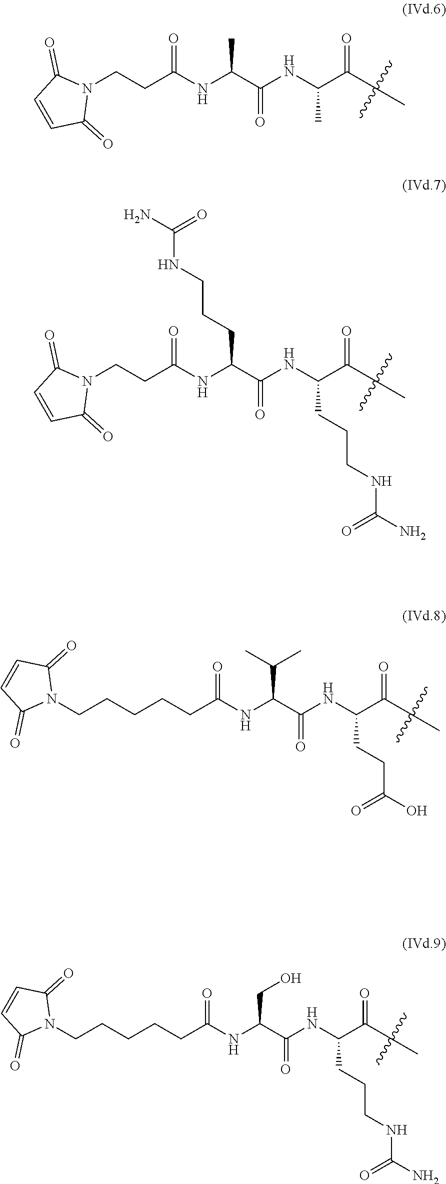 [SMILES: *C(=O)[C@H](C)NC(=O)[C@H](C)NC(=O)CCN1C(=O)C=CC1=O ; *C(=O)[C@H](CCCNC(N)=O)NC(=O)[C@H](CCCNC(N)=O)NC(=O)CCN1C(=O)C=CC1=O ; *C(=O)[C@H](CCC(=O)O)NC(=O)[C@@H](NC(=O)CCCCCN1C(=O)C=CC1=O)C(C)C ; *C(=O)[C@H](CCCNC(N)=O)NC(=O)[C@H](CO)NC(=O)CCCCCN1C(=O)C=CC1=O]

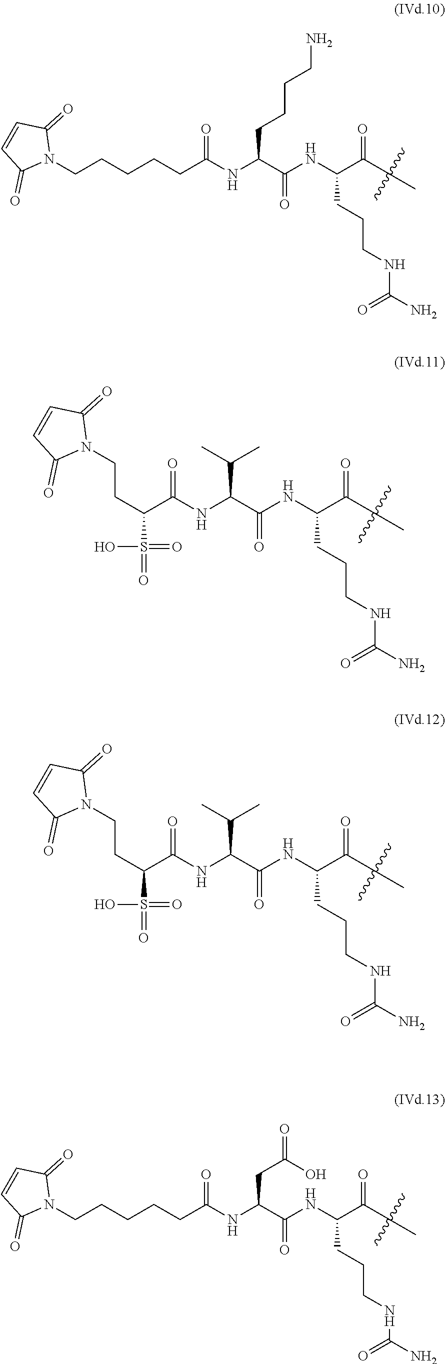 [SMILES: *C(=O)[C@H](CCCNC(N)=O)NC(=O)[C@H](CCCCN)NC(=O)CCCCCN1C(=O)C=CC1=O ; *C(=O)[C@H](CCCNC(N)=O)NC(=O)[C@@H](NC(=O)[C@@H](CCN1C(=O)C=CC1=O)S(=O)(=O)O)C(C)C ; *C(=O)[C@H](CCCNC(N)=O)NC(=O)[C@@H](NC(=O)[C@H](CCN1C(=O)C=CC1=O)S(=O)(=O)O)C(C)C ; *C(=O)[C@H](CCCNC(N)=O)NC(=O)[C@H](CC(=O)O)NC(=O)CCCCCN1C(=O)C=CC1=O]

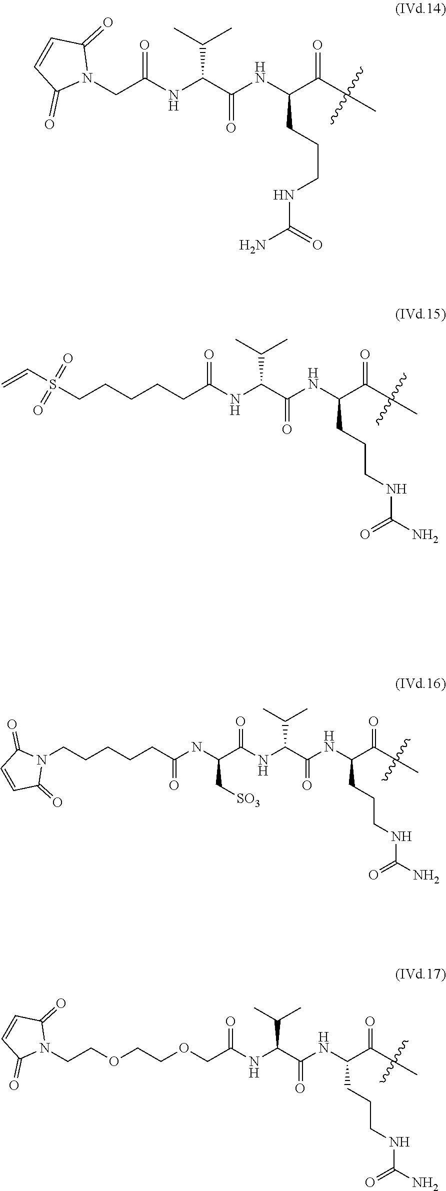

In certain embodiments, the ADC linker comprising structural formula (IVa), (IVb), (IVc), or (IVd) further comprises a carbonate moiety cleavable by exposure to an acidic medium. In particular embodiments, the ADC linker is attached through an oxygen to a cytotoxic and/or cytostatic agent.

7.13.2.2. Non-Cleavable Linkers

Although cleavable ADC linkers can provide certain advantages, the ADC linkers comprising the ADCs need not be cleavable. For noncleavable ADC linkers, the release of drug does not depend on the differential properties between the plasma and some cytoplasmic compartments. The release of the drug is postulated to occur after internalization of the ADC via antigen-mediated endocytosis and delivery to lysosomal compartment, where the MBM is degraded to the level of amino acids through intracellular proteolytic degradation. This process releases a drug derivative, which is formed by the drug, the ADC linker, and the amino acid residue to which the ADC linker was covalently attached. The amino acid drug metabolites from conjugates with noncleavable ADC linkers are more hydrophilic and generally less membrane permeable, which leads to less bystander effects and less nonspecific toxicities compared to conjugates with a cleavable ADC linker. In general, ADCs with noncleavable ADC linkers have greater stability in circulation than ADCs with cleavable ADC linkers. Non-cleavable ADC linkers can be alkylene chains, or maybe polymeric in natures, such as, for example, based upon polyalkylene glycol polymers, amide polymers, or can include segments of alkylene chains, polyalkylene glocols and/or amide polymers.

A variety of non-cleavable ADC linkers used to link drugs to MBMs has been described. See, Jeffrey et al., 2006, Bioconjug. Chem. 17; 831-840; Jeffrey et al., 2007, Bioorg. Med. Chem. Lett. 17:2278-2280; and Jiang et al., 2005, J. Am. Chem. Soc. 127:11254-11255. All of these ADC linkers can be included in the ADCs of the disclosure.

In certain embodiments, the ADC linker is non-cleavable in vivo, for example an ADC linker according to structural formula (VIa), (VIb), (VIc) or (VId) (as illustrated, the ADC linkers include a group suitable for covalently linking the ADC linker to a MBM:

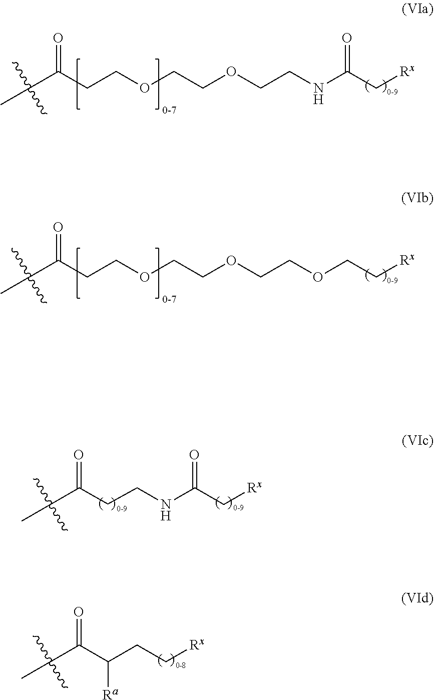

or salts thereof, wherein: R^{a} is selected from hydrogen, alkyl, sulfonate and methyl sulfonate; R^{x} is a moiety including a functional group capable of covalently linking the ADC linker to a MBM; and [symbol] represents the point of attachment of the ADC linker to a cytotoxic and/or cytostatic agent.

Specific exemplary embodiments of ADC linkers according to structural formula (VIa)-(VId) that can be included in the ADCs include the ADC linkers illustrated below (as illustrated, the ADC linkers include a group suitable for covalently linking the ADC linker to a MBM, and [symbol] represents the point of attachment to a cytotoxic and/or cytostatic agent):

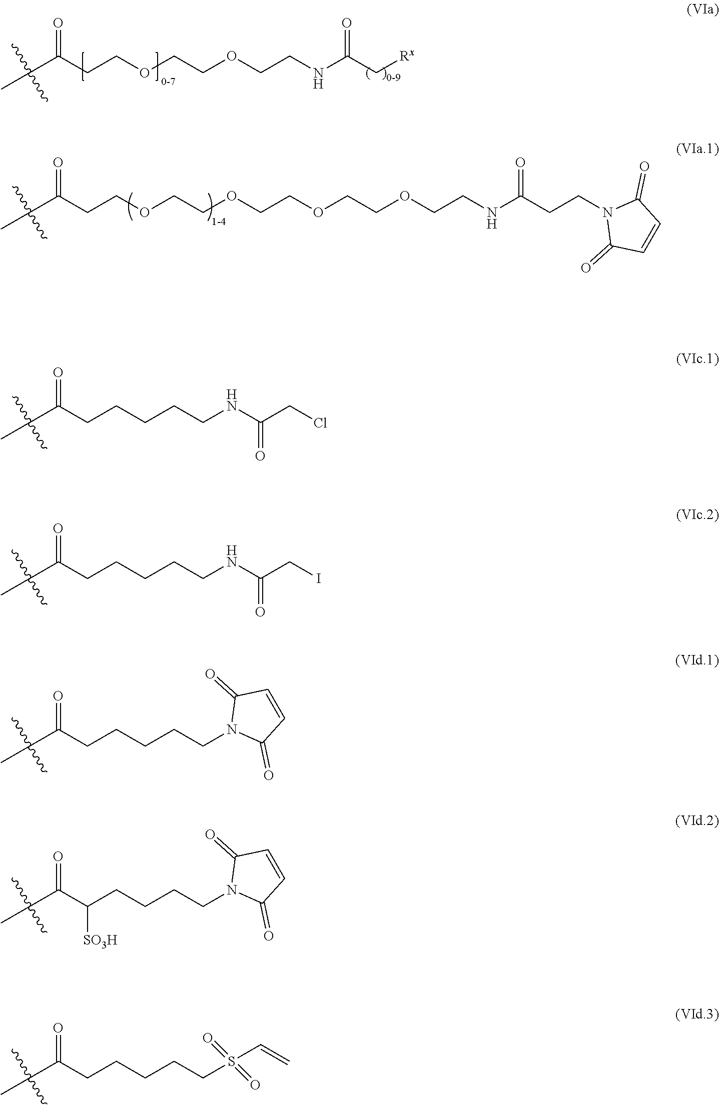

7.13.2.3. Groups Used to Attach Linkers to MBMs

A variety of groups can be used to attach ADC linker-drug synthons to MBMs to yield ADCs. Attachment groups can be electrophilic in nature and include: maleimide groups, activated disulfides, active esters such as NHS esters and HOBt esters, haloformates, acid halides, alkyl and benzyl halides such as haloacetamides. As discussed below, there are also emerging technologies related to “self-stabilizing” maleimides and “bridging disulfides” that can be used in accordance with the disclosure. The specific group used will depend, in part, on the site of attachment to the MBM.

One example of a “self-stabilizing” maleimide group that hydrolyzes spontaneously under MBM conjugation conditions to give an ADC species with improved stability is depicted in the schematic below. See US20130309256 A1; also Lyon et al., Nature Biotech published online, doi:10.1038/nbt.2968.

### Normal System:

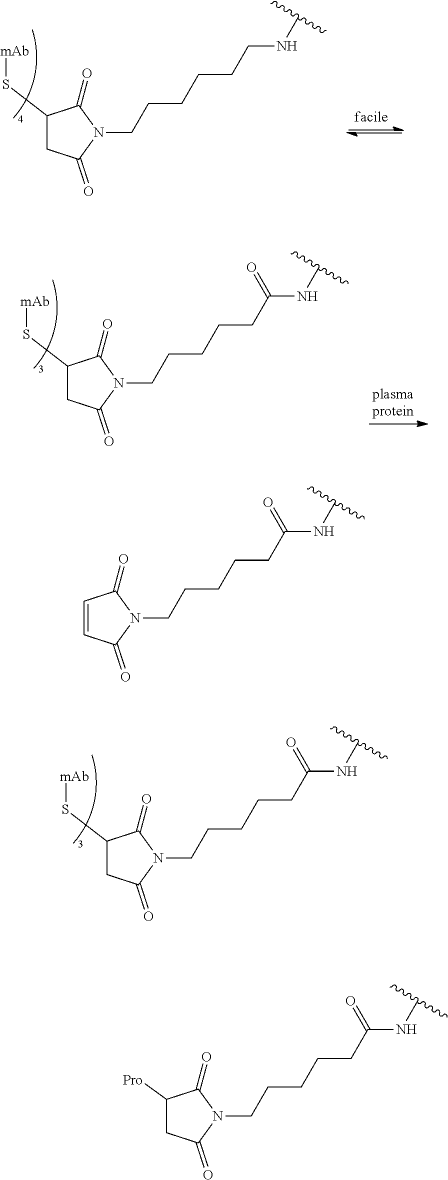

Leads to “DAR loss” over time

### SGN MaIDPR (Maleimido Dipropylamino) System:

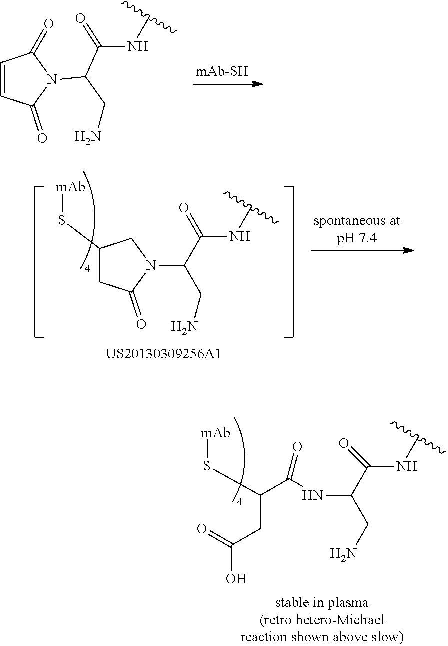

Polytherics has disclosed a method for bridging a pair of sulfhydryl groups derived from reduction of a native hinge disulfide bond. See, Badescu et al., 2014, Bioconjugate Chem. 25:1124-1136. The reaction is depicted in the schematic below. An advantage of this methodology is the ability to synthesize enriched DAR4 ADCs by full reduction of IgGs (to give four pairs of sulfhydryls) followed by reaction with four equivalents of the alkylating agent. ADCs containing “bridged disulfides” are also said to have increased stability.

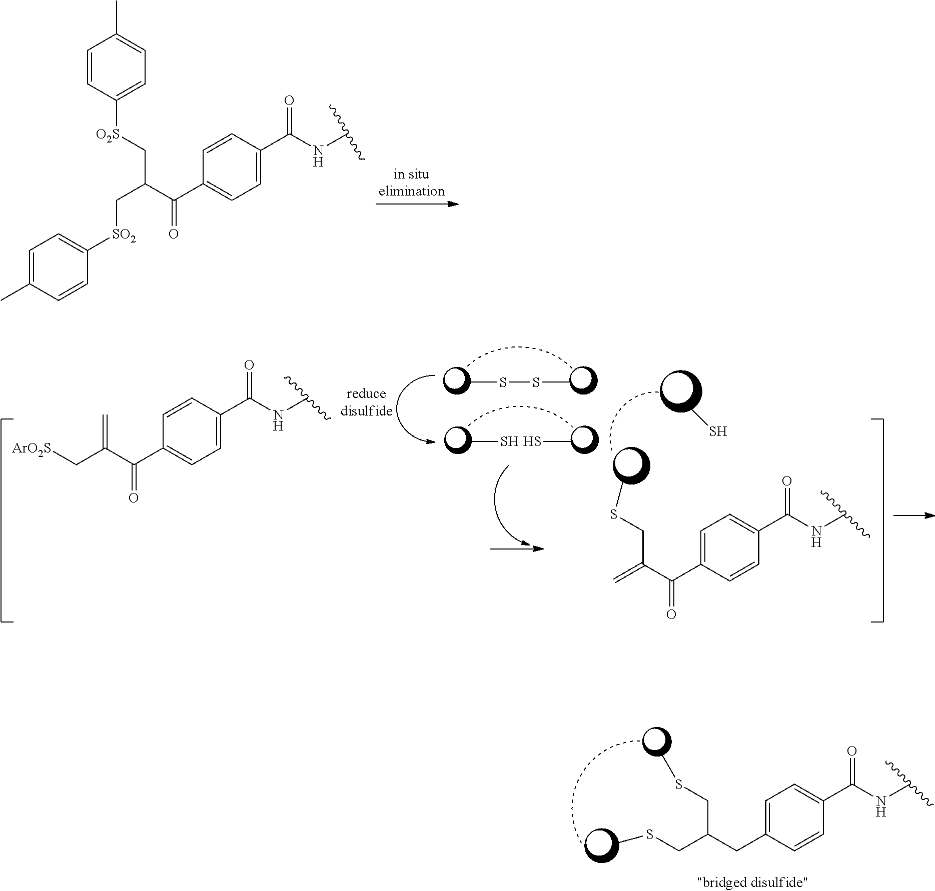

Similarly, as depicted below, a maleimide derivative (1, below) that is capable of bridging a pair of sulfhydryl groups has been developed. See WO2013/085925.

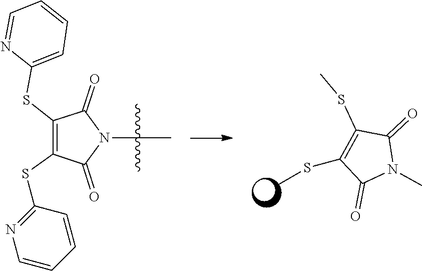

7.13.2.4. ADC Linker Selection Considerations

As is known by skilled artisans, the ADC linker selected for a particular ADC can be influenced by a variety of factors, including but not limited to, the site of attachment to the MBM (e.g., lys, cys or other amino acid residues), structural constraints of the drug pharmacophore and the lipophilicity of the drug. The specific ADC linker selected for an ADC should seek to balance these different factors for the specific MBM/drug combination. For a review of the factors that are influenced by choice of ADC linkers in ADCs, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013.

For example, ADCs have been observed to effect killing of bystander antigen-negative cells present in the vicinity of the antigen-positive tumor cells. The mechanism of bystander cell killing by ADCs has indicated that metabolic products formed during intracellular processing of the ADCs can play a role. Neutral cytotoxic metabolites generated by metabolism of the ADCs in antigen-positive cells appear to play a role in bystander cell killing while charged metabolites can be prevented from diffusing across the membrane into the medium and therefore cannot affect bystander killing. In certain embodiments, the ADC linker is selected to attenuate the bystander killing effect caused by cellular metabolites of the ADC. In certain embodiments, the ADC linker is selected to increase the bystander killing effect.

The properties of the ADC linker can also influence aggregation of the ADC under conditions of use and/or storage. Typically, ADCs reported in the literature contain no more than 3-4 drug molecules per antibody molecule (see, e.g., Chari, 2008, Acc Chem Res 41:98-107). Attempts to obtain higher drug-to-antibody ratios (“DAR”) often failed, particularly if both the drug and the ADC linker were hydrophobic, due to aggregation of the ADC (King et al., 2002, J Med Chem 45:4336-4343; Hollander et al., 2008, Bioconjugate Chem 19:358-361; Burke et al., 2009 Bioconjugate Chem 20:1242-1250). In many instances, DARs higher than 3-4 could be beneficial as a means of increasing potency. In instances where the cytotoxic and/or cytostatic agent is hydrophobic in nature, it can be desirable to select ADC linkers that are relatively hydrophilic as a means of reducing ADC aggregation, especially in instances where DARS greater than 3-4 are desired. Thus, in certain embodiments, the ADC linker incorporates chemical moieties that reduce aggregation of the ADCs during storage and/or use. An ADC linker can incorporate polar or hydrophilic groups such as charged groups or groups that become charged under physiological pH to reduce the aggregation of the ADCs. For example, an ADC linker can incorporate charged groups such as salts or groups that deprotonate, e.g., carboxylates, or protonate, e.g., amines, at physiological pH.

Exemplary polyvalent ADC linkers that have been reported to yield DARs as high as 20 that can be used to link numerous cytotoxic and/or cytostatic agents to a MBM are described in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640.

In particular embodiments, the aggregation of the ADCs during storage or use is less than about 10% as determined by size-exclusion chromatography (SEC). In particular embodiments, the aggregation of the ADCs during storage or use is less than 10%, such as less than about 5%, less than about 4%, less than about 3%, less than about 2%, less than about 1%, less than about 0.5%, less than about 0.1%, or even lower, as determined by size-exclusion chromatography (SEC).

7.13.3. Methods of Making ADCs

The ADCs can be synthesized using chemistries that are well known. The chemistries selected will depend upon, among other things, the identity of the cytotoxic and/or cytostatic agent(s), the ADC linker and the groups used to attach ADC linker to the MBM. Generally, ADCs according to formula (I) can be prepared according to the following scheme:

D-L-R^{x}+Ab-R^{y}→[D-L-XY]ₙ-Ab (I)

Where D, L, Ab, XY and n are as previously defined, and R^{x} and R^{y} represent complementary groups capable of forming a covalent linkages with one another, as discussed above.

The identities of groups R^{x} and R^{y} will depend upon the chemistry used to link synthon D-L-R^{x} to the MBM. Generally, the chemistry used should not alter the integrity of the MBM, for example its ability to bind its target. Preferably, the binding properties of the conjugated antibody will closely resemble those of the unconjugated MBM. A variety of chemistries and techniques for conjugating molecules to biological molecules and in particular to immunoglobulins, whose components are typically building blocks of the MBMs, are well-known. See, e.g., Amon et al., “Monoclonal Antibodies For Immunotargeting Of Drugs In Cancer Therapy,” in: Monoclonal Antibodies And Cancer Therapy, Reisfeld et al. Eds., Alan R. Liss, Inc., 1985; Hellstrom et al., “Antibodies For Drug Delivery,” in: Controlled Drug Delivery, Robinson et al. Eds., Marcel Dekker, Inc., 2nd Ed. 1987; Thorpe, “Antibody Carriers Of Cytotoxic Agents In Cancer Therapy: A Review,” in: Monoclonal Antibodies '84: Biological And Clinical Applications, Pinchera et al., Eds., 1985; “Analysis, Results, and Future Prospective of the Therapeutic Use of Radiolabeled Antibody In Cancer Therapy,” in: Monoclonal Antibodies For Cancer Detection And Therapy, Baldwin et al., Eds., Academic Press, 1985; Thorpe et al., 1982, Immunol. Rev. 62:119-58; PCT publication WO 89/12624. Any of these chemistries can be used to link the synthons to a MBM.

A number of functional groups R^{x} and chemistries useful for linking synthons to accessible lysine residues are known, and include by way of example and not limitation NHS-esters and isothiocyanates.

A number of functional groups R^{x} and chemistries useful for linking synthons to accessible free sulfhydryl groups of cysteine residues are known, and include by way of example and not limitation haloacetyls and maleimides.

However, conjugation chemistries are not limited to available side chain groups. Side chains such as amines can be converted to other useful groups, such as hydroxyls, by linking an appropriate small molecule to the amine. This strategy can be used to increase the number of available linking sites on the antibody by conjugating multifunctional small molecules to side chains of accessible amino acid residues of the MBM. Functional groups R^{x} suitable for covalently linking the synthons to these “converted” functional groups are then included in the synthons.

The MBM can also be engineered to include amino acid residues for conjugation. An approach for engineering MBMs to include non-genetically encoded amino acid residues useful for conjugating drugs in the context of ADCs is described by Axup et al., 2012, Proc Natl Acad Sci USA. 109(40):16101-16106, as are chemistries and functional group useful for linking synthons to the non-encoded amino acids.

Typically, the synthons are linked to the side chains of amino acid residues of the MBM, including, for example, the primary amino group of accessible lysine residues or the sulfhydryl group of accessible cysteine residues. Free sulfhydryl groups can be obtained by reducing interchain disulfide bonds.

For linkages where R^{y} is a sulfhydryl group (for example, when R^{x} is a maleimide), the MBM is generally first fully or partially reduced to disrupt interchain disulfide bridges between cysteine residues.

Cysteine residues that do not participate in disulfide bridges can engineered into a MBM by modification of one or more codons. Introducing these unpaired cysteines yields a sulfhydryl group suitable for conjugation. Preferred positions for incorporating engineered cysteines include, by way of example and not limitation, positions S112C, S113C, A114C, S115C, A176C, S180C, S252C, V286C, V292C, S357C, A359C, S398C, S428C (Kabat numbering) on the human IgG₁ heavy chain and positions V110C, S114C, S121C, S127C, S168C, V205C (Kabat numbering) on the human Ig kappa light chain (see, e.g., U.S. Pat. Nos. 7,521,541, 7,855,275 and 8,455,622).

As will appreciated by skilled artisans, the number of cytotoxic and/or cytostatic agents linked to a MBM molecule can vary, such that a collection of ADCs can be heterogeneous in nature, where some MBMs contain one linked agent, some two, some three, etc. (and some none). The degree of heterogeneity will depend upon, among other things, the chemistries used for linking the cytotoxic and/or cytostatic agents. For example, where the MBMs are reduced to yield sulfhydryl groups for attachment, heterogeneous mixtures of MBMs having 0, 2, 4, 6 or 8 linked agents per molecule are often produced. Furthermore, by limiting the molar ratio of attachment compound, MBMs having 0, 1, 2, 3, 4, 5, 6, 7 or 8 linked agents per molecule are often produced. Thus, it will be understood that depending upon context, stated drug-antibody ratios (DARs) can be averages for a collection of MBMs. For example, “DAR4” can refer to an ADC preparation that has not been subjected to purification to isolate specific DAR peaks and can comprise a heterogeneous mixture of ADC molecules having different numbers of cytostatic and/or cytotoxic agents attached per MBM (e.g., 0, 2, 4, 6, 8 agents per MBM), but has an average drug-to-MBM ratio of 4. Similarly, in some embodiments, “DAR2” refers to a heterogeneous ADC preparation in which the average drug-to-MBM ratio is 2.

When enriched preparations are desired, MBMs having defined numbers of linked cytotoxic and/or cytostatic agents can be obtained via purification of heterogeneous mixtures, for example, via column chromatography, e.g., hydrophobic interaction chromatography.

Purity can be assessed by a variety of methods, as is known in the art. As a specific example, an ADC preparation can be analyzed via HPLC or other chromatography and the purity assessed by analyzing areas under the curves of the resultant peaks.

### 7.14. Pharmaceutical Compositions

The CD3 binding molecules (e.g., MBMs) (as well as their conjugates; references to CD3 binding molecules, e.g., MBMs, in this disclosure also refers to conjugates comprising the CD binding molecules, such as ADCs, unless the context dictates otherwise) can be formulated as pharmaceutical compositions comprising the CD3 binding molecules, for example containing one or more pharmaceutically acceptable excipients or carriers. To prepare pharmaceutical or sterile compositions comprising the CD3 binding molecules (e.g., MBMs) of the present disclosure a CD3 binding molecules preparation can be combined with one or more pharmaceutically acceptable excipient or carrier.

For example, formulations of CD3 binding molecules (e.g., MBMs) can be prepared by mixing CD3 binding molecules with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.),1993, Pharmaceutical Dosage Forms: General Medications, Marcel Dekker, N.Y.; Lieberman, et al. (eds.), 1990, Pharmaceutical Dosage Forms: Tablets, Marcel Dekker, N.Y.; Lieberman, et al. (eds.), 1990, Pharmaceutical Dosage Forms: Disperse Systems, Marcel Dekker, N.Y.; Weiner and Kotkoskie, 2000, Excipient Toxicity and Safety, Marcel Dekker, Inc., New York, N.Y.).

Selecting an administration regimen for a CD3 binding molecule (e.g., MBM) depends on several factors, including the serum or tissue turnover rate of the CD3 binding molecule, the level of symptoms, the immunogenicity of the CD3 binding molecule, and the accessibility of the target cells. In certain embodiments, an administration regimen maximizes the amount of CD3 binding molecule delivered to the subject consistent with an acceptable level of side effects. Accordingly, the amount of CD3 binding molecule delivered depends in part on the particular CD3 binding molecule and the severity of the condition being treated. Guidance in selecting appropriate doses of antibodies and small molecules are available (see, e.g., Wawrzynczak, 1996, Antibody Therapy, Bios Scientific Pub. Ltd, Oxfordshire, UK; Kresina (ed.), 1991, Monoclonal Antibodies, Cytokines and Arthritis, Marcel Dekker, New York, N.Y.; Bach (ed.), 1993, Monoclonal Antibodies and Peptide Therapy in Autoimmune Diseases, Marcel Dekker, New York, N.Y.; Baert et al., 2003, New Engl. J. Med. 348:601-608; Milgrom et al., 1999, New Engl. J. Med. 341:1966-1973; Slamon et al., 2001, New Engl. J. Med. 344:783-792; Beniaminovitz et al., 2000, New Engl. J. Med. 342:613-619; Ghosh et al., 2003, New Engl. J. Med. 348:24-32; Lipsky et al., 2000, New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the CD3 binding molecules (e.g., MBMs) in the pharmaceutical compositions of the present disclosure can be varied to obtain an amount of the CD3 binding molecule which is effective to achieve the desired therapeutic response for a particular subject, composition, and mode of administration, without being toxic to the subject. The selected dosage level will depend upon a variety of pharmacokinetic factors including the activity of the particular CD3 binding molecule, the route of administration, the time of administration, the rate of excretion of the particular CD3 binding molecule being employed, the duration of the treatment, other agents (e.g., active agents such as therapeutic drugs or compounds and/or inert materials used as carriers) in combination with the particular CD3 binding molecule employed, the age, sex, weight, condition, general health and prior medical history of the subject being treated, and like factors known in the medical arts.

Compositions comprising the CD3 binding molecules (e.g., MBMs) can be provided by continuous infusion, or by doses at intervals of, e.g., one day, one week, or 1-7 times per week. Doses can be provided intravenously, subcutaneously, topically, orally, nasally, rectally, intramuscular, intracerebrally, or by inhalation. A specific dose protocol is one involving the maximal dose or dose frequency that avoids significant undesirable side effects.

An effective amount for a particular subject can vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The route of administration can be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., 1983, Biopolymers 22:547-556; Langer et al., 1981, J. Biomed. Mater. Res. 15:167-277; Langer, 1982, Chem. Tech. 12:98-105; Epstein et al., 1985, Proc. Natl. Acad. Sci. USA 82:3688-3692; Hwang et al., 1980, Proc. Natl. Acad. Sci. USA 77:4030-4034; U.S. Pat. Nos. 6,350,466 and 6,316,024). Where necessary, the composition can also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903.

A composition of the present disclosure can also be administered via one or more routes of administration using one or more of a variety of methods known in the art. As will be appreciated by the skilled artisan, the route and/or mode of administration will vary depending upon the desired results. Selected routes of administration for CD3 binding molecules (e.g., MBMs) include intravenous, intramuscular, intradermal, intraperitoneal, subcutaneous, spinal or other general routes of administration, for example by injection or infusion. General administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition can be administered via a non-general route, such as a topical, epidermal or mucosal route of administration, for example, intranasally, orally, vaginally, rectally, sublingually or topically. In one embodiment, the CD3 binding molecules (e.g., MBMs) are administered by infusion. In another embodiment, the CD3 binding molecules (e.g., MBMs) are administered subcutaneously.

If the CD3 binding molecules (e.g., MBMs) are administered in a controlled release or sustained release system, a pump can be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the disclosure (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more MBMs of the disclosure. See, e.g., U.S. Pat. No. 4,526,938, PCT publication WO 91/05548, PCT publication WO 96/20698, Ning et al., 1996, Radiotherapy & Oncology 39:179-189, Song et al., 1995, PDA Journal of Pharmaceutical Science & Technology 50:372-397, Cleek et al., 1997, Pro. Intl Symp. Control. Rel. Bioact. Mater. 24:853-854, and Lam et al., 1997, Proc. Intl Symp. Control Rel. Bioact. Mater. 24:759-760.

If the CD3 binding molecules (e.g., MBMs) are administered topically, they can be formulated in the form of an ointment, cream, transdermal patch, lotion, gel, shampoo, spray, aerosol, solution, emulsion, or other form well known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well known in the art.

If the compositions comprising the CD3 binding molecules (e.g., MBMs) are administered intranasally, the CD3 binding molecules can be formulated in an aerosol form, spray, mist or in the form of drops. In particular, prophylactic or therapeutic agents for use according to the present disclosure can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit can be determined by providing a valve to deliver a metered amount. Capsules and cartridges (composed of, e.g., gelatin) for use in an inhaler or insufflator can be formulated containing a powder mix of the compound and a suitable powder base such as lactose or starch.

The CD3 binding molecules (e.g., MBMs) can be administered in combination therapy regimens, as described in Section 7.16.

In certain embodiments, the CD3 binding molecules (e.g., MBMs) can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., Ranade, 1989, J. Clin. Pharmacol. 29:685). Exemplary targeting moieties include folate or biotin (see, e.g., U.S. Pat. No. 5,416,016 to Low et al.); mannosides (Umezawa et al., 1988, Biochem. Biophys. Res. Commun. 153:1038); antibodies (Bloeman et al., 1995, FEBS Lett. 357:140; Owais et al., 1995, Antimicrob. Agents Chemother. 39:180); surfactant protein A receptor (Briscoe et al., 1995, Am. J. Physiol. 1233:134); p 120 (Schreier et al., 1994, J. Biol. Chem. 269:9090); see also Keinanen and Laukkanen, 1994, FEBS Lett. 346:123; Killion and Fidler,1994, Immunomethods 4:273.

When used in combination therapy, e.g., as described in Section 7.16, a CD3 binding molecule (e.g., MBM) and one or more additional agents can be administered to a subject in the same pharmaceutical composition. Alternatively, the CD3 binding molecule and the additional agent(s) of the combination therapies can be administered concurrently to a subject in separate pharmaceutical compositions.

The therapeutic methods described herein can further comprise carrying a “companion diagnostic” test whereby a sample from a subject who is a candidate for therapy with a CD3 binding molecule (e.g., MBM) is tested for the expression of the TAA targeted by ABM2 and/or is tested for the expression of the TAA targeted by ABM3 (when ABM3 targets a TAA). The companion diagnostic test can be performed prior to initiating therapy with a CD3 binding molecule (e.g., MBM) and/or during a therapeutic regimen with a CD3 binding molecule (e.g., MBM) to monitor the subject's continued suitability for CD3 binding molecule therapy. The agent used in the companion diagnostic can be the CD3 binding molecule (e.g., MBM) itself or another diagnostic agent, for example a labeled monospecific antibody against the TAA recognized by ABM2 (or ABM3) or a nucleic acid probe to detect TAA RNA. The sample that can be tested in a companion diagnostic assay can be any sample in which the cells targeted by the CD3 binding molecule (e.g., MBM) can be present, from example a tumor (e.g., a solid tumor) biopsy, lymph, stool, urine, blood or any other bodily fluid that might contain circulating tumor cells.

### 7.15. Therapeutic Indications

The CD3 binding molecules (e.g., MBMs) can be used in the treatment of immune (e.g., autoimmune) and inflammatory disease as well as proliferative diseases such as cancer.

7.15.1. Cancer

The MBMs can be used in the treatment of any proliferative disorder (e.g., cancer) that expresses a TAA targeted by such MBMs. In particular embodiments, the cancer is HER2+ cancer, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, or Wilms tumor.

Table 18 below shows exemplary indications that MBMs targeting particular TAAs can be used against.

TABLE 18
Examples of Tumor-Associated Antigen Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, prostate, ovarian, pancreatic, |
| | colorectal cancers |
| BCMA | multiple myeloma, leukemia (e.g., acute lymphoblastic leukemia (“ALL”), |
| | acute myeloid leukemia (“AML”), chronic lymphocytic leukemia (“CLL”), |
| | chronic myeloid leukemia (“CML”) and hairy cell leukemia (“HCL”)); |
| | lymphoma (e.g., Hodgkin's lymphoma, non-Hodgkin's lymphoma, including |
| | diffuse large B-cell lymphoma (“DLBCL”)) |
| Cadherin 17 | gastric, pancreatic, and colorectal adenocarcinomas |
| CAIX | clear-cell renal cell carcinoma, hypoxic solid tumors, head and neck |
| | squamous carcinoma |
| CD123 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD171 | neuroblastoma, paraganglioma |
| CD179a | B cell malignancies |
| CD19 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD20 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD22 | leukemia (e.g., ALL, CLL, AML, CML, HCL); lymphoma (e.g., Hodgkin's |
| | lymphoma, non-Hodgkin's lymphoma, e.g., DLBCL); multiple myeloma; |
| | lung cancer |
| CD24 | ovarian, breast, prostate, bladder, renal, non-small cell carcinomas |
| CD30 | anaplastic large cell lymphoma, embryonal carcinoma, Hodgkin Lymphoma |
| CD32b | B cell malignancies, gastric, pancreatic, esophageal, glioblastoma, breast, |
| | colorectal |
| CD33 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD38 | leukemia (e.g., ALL, CLL, AML, CML, HCL); lymphoma (e.g., Hodgkin's |
| | lymphoma, non-Hodgkin's lymphoma, e.g., DLBCL); multiple myeloma |
| CD44v6 | colon cancer, head and neck small cell carcinoma |
| CD97 | B cell malignancies, gastric, pancreatic, esophageal, glioblastoma, breast, |
| | colorectal |
| CEA | colorectal carcinoma, gastric carcinoma, pancreatic carcinoma, lung |
| | cancer, breast cancer, medullary thyroid carcinoma |
| CLDN6 | ovarian, breast, lung cancer |
| CLL-1 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CS1 | multiple myeloma |
| EGFR | squamous cell carcinoma of lung, anal cancer, glioblastoma, epithelian |
| | tumors of head and neck, colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung adenocarcinoma, non-small cell lung |
| (Her2/neu) | cancer, uterine cancer, uterine serous endometrial carcinoma, salivary duct |
| | carcinoma, |
| FAP | pancreatic cancer, colorectal cancer, metastasis, epithelial cancers, soft |
| | tissue sarcomas |
| FCRL5 | multiple myeloma |
| FLT3 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| Folate receptor | ovarian, breast, renal, lung, colorectal, brain cancers |
| alpha | |
| Folate receptor | ovarian cancer |
| beta | |
| Fucosyl GM1 | AML, myeloma |
| GD2 | malignant melanoma, neuroblastoma |
| GD3 | melanoma |
| GloboH | ovarian, gastric, prostate, lung, breast, and pancreatic cancers |
| gp100 | melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER3 | colon and gastric cancers |
| HMWMAA | melanoma, glioblastoma, breast cancer |
| IGF-I receptor | breast, prostate, lung cancers |
| IL-11Ra | papillary thyroid cancer, osteosarcoma, colorectal adenocarcinoma, |
| | lymphocytic leukemia |
| IL-13Ra2 | renal cell carcinoma, prostate cancer, gliomas, head and neck cancer, |
| | astrocytoma |
| KIT | myeloid leukemia, kaposi's sarcoma, erythroleukemia, gastrointestinal |
| | stromal tumors |
| KLRG2 | breast cancers, lung cancers and ovarian cancers. |
| LewisY | squamous cell lung carcinoma, lung adenocarcinoma, ovarian carcinoma, |
| | and colorectal adenocarcinoma |
| LMP2 | prostate cancer, Hodgkin's lymphoma, nasopharyngeal carcinoma |
| LRP6 | breast cancer |
| LY6K | breast, lung, ovarian, and cervical cancer |
| LYPD8 | colorectal and gastric cancers |
| Mesothelin | mesothelioma, pancreatic cancer, ovarian cancer, stomach cancer, lung |
| | cancer, endometrial cancer. |
| MUC1 | breast and ovarian cancers, lung, stomach, pancreatic, prostate cancers |
| NCAM | melanoma, Wilms' tumor, small cell lung cancer, neuroblastoma, myeloma, |
| | paraganglioma, pancreatic acinar cell carcinoma, myeloid leukemia |
| NY-BR-1 | breast cancer |
| o-acetyl GD2 | neuroblastoma, melanoma |
| OR51E2 | prostate cancer |
| PANX3 | osteosarcoma |
| PLAC1 | hepatocellular carcinoma |
| Polysialic acid | small cell lung cancer |
| PDGFR-beta | myelomonocytic leukemia, chronic myeloid leukemia, acute myelogenous |
| | leukemia, acute lymphoblastic leukemia |
| PRSS21 | colon cancer, testicular cancer, ovarian cancer |
| PSCA | prostate cancer, gastric and bladder cancers |
| PSMA | prostate cancer, |
| ROR1 | metastatic cancers, chronic lymphocytic leukemia, solid tumors in lung, |
| | breast, ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial ovarian carcinoma |
| TACSTD2 | carcinomas, e.g., non-small-cell lung cancer |
| TAG72 | ovarian, breast, colon, lung, pancreatic cancers, gastric cancer |
| TEM1/CD248 | colorectal cancer |
| TEM7R | colorectal cancer |
| Tn | colorectal, breast cancers, cervical, lung, stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell carcinoma, colorectal cancer, |
| | medullary thyroid carcinoma |

Accordingly, the present disclosure provides methods of treating cancer comprising administering to a subject suffering from cancer a MBM which binds to a TAA or combination of TAAs expressed on that type of cancer. In some embodiments, a MBM that targets a TAA identified in Table 18 is can be administered to a subject afflicted with a cancer that Table 18 indicates expressed the TAA. By way of example and not limitation, a MBM that targets EPCAM or folate receptor alpha can be administered to a subject afflicted with colorectal cancer, a MBM that targets BCMA or CD19 can be administered to a subject afflicted with a blood cancer such as multiple myeloma, a MBM that targets PSCA or PCMA can be administered a subject afflicted with prostate cancer, a MBM that targets tyrosinase or GP3 can be administered to a subject afflicted with melanoma, a MBM that targets CD33, CLL-1 or FLT3 can be administered to a subject afflicted with a blood cancer such as acute myeloid leukemia.

The MBMs (e.g., TBMs) can be used in the treatment of any proliferative disorder (e.g., cancer) that expresses a TAA described in Section 7.10 or combination of TAAs described in Section 7.10 (e.g., a cancer characterized by cancerous cells expressing two TAAs on the same cancerous cell or a cancer characterized by cancerous cells expressing a first TAA and a second TAA on different cancerous cells). In specific embodiments, the cancer is a B cell malignancy. Exemplary types of B cell malignancies that may be targeted include Hodgkin's lymphomas, non-Hodgkin's lymphomas (NHLs), and multiple myeloma. Examples of NHLs include diffuse large B-cell lymphoma (DLBCL), follicular lymphoma, chronic lymphocytic leukemia (CLL)/small lymphocytic lymphoma (SLL), mantle cell lymphoma (MCL), marginal zone lymphomas, Burkitt lymphoma, lymphoplasmacytic lymphoma (Waldenstrom macroglobulinemia), hairy cell leukemia, primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, and primary effusion lymphoma.

In some embodiments, the MBMs are used to treat Hodgkin's lymphoma. In some embodiments, the MBMs are used to treat non-Hodgkin's lymphoma. In some embodiments, the MBMs are used to treat diffuse large B-cell lymphoma (DLBCL). In some embodiments, the MBMs are used to treat follicular lymphoma. In some embodiments, the MBMs are used to treat chronic lymphocytic leukemia (CLL)/small lymphocytic lymphoma (SLL). In some embodiments, the MBMs are used to treat mantle cell lymphoma (MCL). In some embodiments, the MBMs are used to treat marginal zone lymphoma. In some embodiments, the MBMs are used to treat Burkitt lymphoma. In some embodiments, the MBMs are used to treat lymphoplasmacytic lymphoma (Waldenstrom macroglobulinemia). In some embodiments, the MBMs are used to treat hairy cell leukemia. In some embodiments, the MBMs are used to treat primary central nervous system (CNS) lymphoma. In some embodiments, the MBMs are used to treat primary mediastinal large B-cell lymphoma. In some embodiments, the MBMs are used to treat mediastinal grey-zone lymphoma (MGZL). In some embodiments, the MBMs are used to treat splenic marginal zone B-cell lymphoma. In some embodiments, the MBMs are used to treat extranodal marginal zone B-cell lymphoma of MALT. In some embodiments, the MBMs are used to treat nodal marginal zone B-cell lymphoma. In some embodiments, the MBMs are used to treat primary effusion lymphoma. In some embodiments, the MBMs are used to treat a plasmacytic dendritic cell neoplasm. In some embodiments, the MBMs are used to treat multiple myeloma.

7.15.2. Autoimmune Diseases

The CD3 binding molecules (e.g., MBMs) can be used in the treatment of autoimmune disorders, which can result from the loss of B-cell tolerance and the inappropriate production of autoantibodies. Autoimmune disorders that can be treated with the CD3 binding molecules include systemic lupus erythematosus (SLE), Sjögren's syndrome, scleroderma, rheumatoid arthritis (RA), juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, pemphigus vulgaris, vitiligo, autoimmune haemolytic anaemia, idiopathic thrombocytopenic purpura, giant cell arteritis, myasthenia gravis, multiple sclerosis (MS) (e.g., relapsing-remitting MS (RRMS)), glomerulonephritis, Goodpasture's syndrome, bullous pemphigoid, colitis ulcerosa, Guillain-Barré syndrome, chronic inflammatory demyelinating polyneuropathy, anti-phospholipid syndrome, narcolepsy, sarcoidosis, and Wegener's granulomatosis.

In some embodiments, the CD3 binding molecules are used to treat systemic lupus erythematosus (SLE). In some embodiments, the CD3 binding molecules are used to treat Sjögren's syndrome. In some embodiments, the CD3 binding molecules are used to treat scleroderma. In some embodiments, the CD3 binding molecules are used to treat rheumatoid arthritis (RA). In some embodiments, the CD3 binding molecules are used to treat juvenile idiopathic arthritis. In some embodiments, the CD3 binding molecules are used to treat graft versus host disease. In some embodiments, the CD3 binding molecules are used to treat dermatomyositis. In some embodiments, the CD3 binding molecules are used to treat type I diabetes mellitus. In some embodiments, the CD3 binding molecules are used to treat Hashimoto's thyroiditis. In some embodiments, the CD3 binding molecules are used to treat Graves's disease. In some embodiments, the CD3 binding molecules are used to treat Addison's disease. In some embodiments, the CD3 binding molecules are used to treat celiac disease. In some embodiments, the CD3 binding molecules are used to treat Crohn's Disease. In some embodiments, the CD3 binding molecules are used to treat pernicious anaemia. In some embodiments, the CD3 binding molecules are used to treat pemphigus vulgaris. In some embodiments, the CD3 binding molecules are used to treat vitiligo. In some embodiments, the CD3 binding molecules are used to treat autoimmune haemolytic anaemia. In some embodiments, the CD3 binding molecules are used to treat idiopathic thrombocytopenic purpura. In some embodiments, the CD3 binding molecules are used to treat giant cell arteritis. In some embodiments, the CD3 binding molecules are used to treat myasthenia gravis. In some embodiments, the CD3 binding molecules are used to treat multiple sclerosis (MS). In some embodiments, the MS is relapsing-remitting MS (RRMS). In some embodiments, the CD3 binding molecules are used to treat glomerulonephritis. In some embodiments, the CD3 binding molecules are used to treat Goodpasture's syndrome. In some embodiments, the CD3 binding molecules are used to treat bullous pemphigoid. In some embodiments, the CD3 binding molecules are used to treat colitis ulcerosa. In some embodiments, the CD3 binding molecules are used to treat Guillain-Barré syndrome. In some embodiments, the CD3 binding molecules are used to treat chronic inflammatory demyelinating polyneuropathy. In some embodiments, the CD3 binding molecules are used to treat anti-phospholipid syndrome. In some embodiments, the CD3 binding molecules are used to treat narcolepsy. In some embodiments, the CD3 binding molecules are used to treat sarcoidosis. In some embodiments, the CD3 binding molecules are used to treat Wegener's granulomatosis.

### 7.16. Combination Therapy

A CD3 binding molecule (e.g., a MBM) can be used in combination other known agents and therapies. For example, the CD3 binding molecules (e.g., MBMs) can be used in treatment regimens in combination with surgery, chemotherapy, antibodies, radiation, peptide vaccines, steroids, cytoxins, proteasome inhibitors, immunomodulatory drugs (e.g., IMiDs), BH3 mimetics, cytokine therapies, stem cell transplant or a combination thereof. Without being bound by theory, it is believed that one of the advantages of the MBMs is that they can circumvent the need for administering separate antibodies, for example to a subject suffering from a B cell malignancy. Accordingly, in certain embodiments, the one or more additional agents do not include an antibody (e.g., rituximab).

For convenience, an agent that is used in combination with a CD3 binding molecule (e.g., a MBM) is referred to herein as an “additional” agent.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” The term “concurrently” is not limited to the administration of therapies (e.g., a MBM and an additional agent) at exactly the same time, but rather it is meant that a pharmaceutical composition comprising a CD3 binding molecule (e.g., MBM) is administered to a subject in a sequence and within a time interval such that the CD3 binding molecule can act together with the additional therapy(ies) to provide an increased benefit than if they were administered otherwise. For example, each therapy can be administered to a subject at the same time or sequentially in any order at different points in time; however, if not administered at the same time, they should be administered sufficiently close in time to provide the desired therapeutic effect.

A CD3 binding molecule (e.g., a MBM) and one or more additional agents can be administered simultaneously, in the same or in separate compositions, or sequentially. For sequential administration, the CD3 binding molecule (e.g., MBM) can be administered first, and the additional agent can be administered second, or the order of administration can be reversed.

The CD3 binding molecule (e.g., MBM) and the additional agent(s) can be administered to a subject in any appropriate form and by any suitable route. In some embodiments, the routes of administration are the same. In other embodiments, the routes of administration are different.

In other embodiments, the delivery of one treatment ends before the delivery of the other treatment begins.

In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The CD3 binding molecules (e.g., MBMs) and/or additional agents can be administered during periods of active disorder, or during a period of remission or less active disease. A CD3 binding molecule (e.g., MBM) can be administered before the treatment with the additional agent(s), concurrently with the treatment with the additional agent(s), post-treatment with the additional agent(s), or during remission of the disorder.

When administered in combination, the CD3 binding molecule (e.g., MBM) and/or the additional agent(s) can be administered in an amount or dose that is higher, lower or the same than the amount or dosage of each agent used individually, e.g., as a monotherapy.

The additional agent(s) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising a CD3 binding molecule (e.g., a MBM) is administered to a subject in a sequence and within a time interval such that the molecules can act together with the additional therapy(ies) to provide an increased benefit than if they were administered otherwise. For example, each therapy can be administered to a subject at the same time or sequentially in any order at different points in time; however, if not administered at the same time, they should be administered sufficiently close in time to provide the desired therapeutic or prophylactic effect. Each therapy can be administered to a subject separately, in any appropriate form and by any suitable route.

The CD3 binding molecule (e.g., MBM) and the additional agent(s) can be administered to a subject by the same or different routes of administration.

The CD3 binding molecules (e.g., MBMs) and the additional agent(s) can be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain instances, the one or more additional agents, are other anti-cancer agents, anti-allergic agents, anti-nausea agents (or anti-emetics), pain relievers, cytoprotective agents, and combinations thereof.

In one embodiment, a CD3 binding molecule (e.g., MBM) can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab, obinutuzumab, ofatumumab, daratumumab, elotuzumab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with the CD3 binding molecules (e.g., MBMs) of the present disclosure include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(SEQ ID NO: 1113), inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

Exemplary BH3 mimetics include venetoclax, ABT-737 (4-{4-[(4′-Chloro-2-biphenylyl)methyl]-1-piperazinyl}-N-[(4-{[(2R)-4-(dimethylamino)-1-(phenylsulfanyl)-2-butanyl]amino}-3-nitrophenyl)sulfonyl]benzamide and navitoclax (formerly ABT-263).

Exemplary gamma secretase inhibitors include compounds of formula (I) or a pharmaceutically acceptable salt thereof;

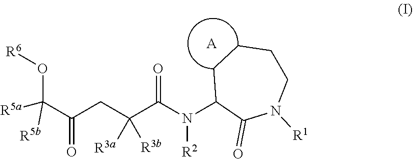

where ring A is aryl or heteroaryl; each of R¹, R², and R⁴ is independently hydrogen, C₁-C₆ alkyl, cycloalkyl, heterocyclyl, cycloalkylalkyl, heterocyclylalkyl, aryl, heteroaryl, aralkyl, or heteroaralkyl, where each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{A}, —SR^{A}, —C(O)OR^{A}, —C(O)N(R^{A})(R^{B}), —N(R^{A})(R^{B}), or —C(NR^{C})N(R^{A})(R^{B}); each R^{3a}, R^{3b}, R^{5a}, and R^{5b} is independently hydrogen, halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, —OR^{A}, —SR^{A}, —C(O)OR^{A}, —C(O)N(R^{A})(R^{B}), —N(R^{A})(R^{B}), or —C(NR^{C})N(R^{A})(R^{B}); R⁶ is hydrogen, C₁-C₆ alkyl, cycloalkyl, heterocyclyl, cycloalkylalkyl, heterocyclylalkyl, aryl, heteroaryl, aralkyl, or heteroaralkyl, where each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy; and each R^{A}, R^{B}, and R^{C} is independently hydrogen, C₁-C₆ alkyl, cycloalkyl, heterocyclyl, cycloalkylalkyl, heterocyclylalkyl, aryl, heteroaryl, aralkyl, or heteroaralkyl, where each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy.

In an embodiment, the compound of formula (I) is a compound described in U.S. Pat. No. 7,468,365. In yet another embodiment, the compound is

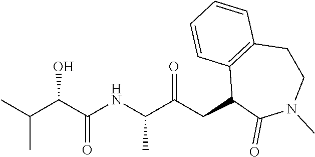

or a pharmaceutically acceptable salt thereof.

The GSI can be a compound of formula (II) or a pharmaceutically acceptable salt thereof;

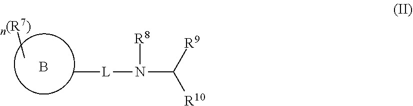

where ring B is aryl or heteroaryl; L is a bond, C₁-C₆ alkylene, —S(O)₂—, —C(O)—, —N(R^{E})(O)C—, or —OC(O)—; each R⁷ is independently halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is independently substituted with 0-6 occurrences of halogen, —OR^{D}, —SR^{D}, —C(O)OR^{D}, —C(O)N(R^{D})(R^{E}), —N(R^{D})(R^{E}), or —C(NR^{F})N(R^{D})(R^{E}); R⁸ is hydrogen, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{D}, —SR^{D}, —C(O)OR^{D}, —C(O)N(R^{D})(R^{E}), —N(R^{D})(R^{E}), or —C(NR^{F})N(R^{D})(R^{E}); each of R⁹ and R¹⁰ is independently hydrogen, halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆alkoxy, cycloalkyl, heterocyclyl, aryl, heteroaryl, aralkyl, or heteroaralkyl is substituted with 0-6 independent occurrences of halogen, —OR^{D}, —SR^{D}, —C(O)OR^{D}, —C(O)N(R^{D})(R^{E}), —N(R^{D})(R^{E}), or —C(NR^{I})N(R^{G})(R^{H}); each R^{D}, R^{E}, and R^{F} is independently hydrogen, C₁-C₆ alkyl, cycloalkyl, heterocyclyl, cycloalkylalkyl, heterocyclylalkyl, aryl, heteroaryl, aralkyl, or heteroaralkyl, where each C C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy; and n is 0, 1, 2, 3, 4, or 5.

In a further embodiment, the compound of formula (II) is a compound described in U.S. Pat. No. 7,687,666. In yet another embodiment, the compound is

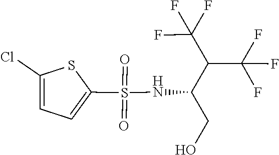 [SMILES: O=S(=O)(N[C@@H](CO)C(C(F)(F)F)C(F)(F)F)c1ccc(Cl)s1]

or a pharmaceutically acceptable salt thereof.

In some embodiments, the GSI is a compound is a compound of formula (III) or a pharmaceutically acceptable salt thereof:

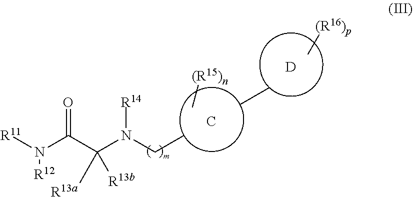

where each of rings C and D is independently aryl or heteroaryl;
each of R¹¹, R¹², and R¹⁴ is independently hydrogen, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{G}, —SR^{G}, —C(O)OR^{G}, —C(O)N(R^{G})(R^{H}), —N(R^{G})(R^{H}), or —C(NR^{I})N(R^{G})(R^{H}); each of R^{13a} and R^{13b} is hydrogen, halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{G}, —SR^{G}, —C(O)OR^{G}, —C(O)N(R^{G})(R^{H}), —N(R^{G})(R^{H}), or —C(NR^{I})N(R^{G})(R^{H}); each R¹⁵ and R¹⁶ is independently halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{G}, —SR^{G}, —C(O)OR^{G}, —C(O)N(R^{G})(R^{H}), —N(R^{G})(R^{H}), or —C(NR^{I})N(R^{G})(R^{H}); each R^{G}, R^{H}, and R¹ is independently hydrogen, C₁-C₆ alkyl, cycloalkyl, heterocyclyl, cycloalkylalkyl, heterocyclylalkyl, aryl, heteroaryl, aralkyl, or heteroaralkyl, where each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy; and each of m, n, and p is independently 0, 1, 2, 3, 4, or 5.

In a further embodiment, the GSI is a compound described in U.S. Pat. No. 8,084,477. In yet another embodiment, the GSI is

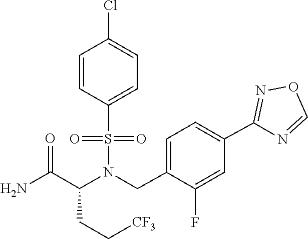 [SMILES: NC(=O)[C@@H](CCC(F)(F)F)N(Cc1ccc(-c2ncon2)cc1F)S(=O)(=O)c1ccc(Cl)cc1]

or a pharmaceutically acceptable salt thereof.

In some embodiments, the GSI is a compound described in U.S. Pat. No. 7,160,875. In some embodiments, the gamma secretase inhibitor is a compound of formula (IV) or a pharmaceutically acceptable salt thereof:

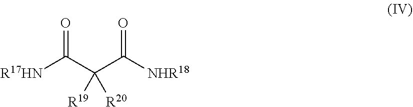

where R¹⁷ is selected from

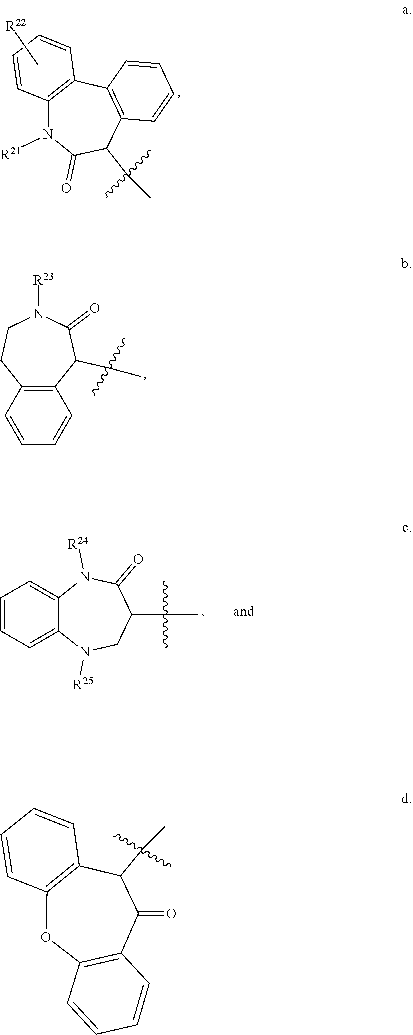

R¹⁸ is lower alkyl, lower alkinyl, —(CH₂)ₙ—O-lower alkyl, —(CH₂)ₙ—S-lower alkyl, —(CH₂)ₙ—CN, —(CR′R″)ₙ—CF₃, —(CR′R″)ₙ—CHF₂, —(CR′R″)ₙ—CH₂F, —(CH₂)ₙ, —C(O)O-lower alkyl, —(CH₂)ₙ-halogen, or is —(CH₂)ₙ-cycloalkyl optionally substituted by one or more substituents selected from the group consisting of phenyl, halogen and CF₃; R′,R″ are each independently hydrogen, lower alkyl, lower alkoxy, halogen or hydroxy; R¹⁹, R²⁰ are each independently hydrogen, lower alkyl, lower alkoxy, phenyl or halogen; R²¹ is hydrogen, lower alkyl, —(CH₂)ₙ—CF₃ or —(CH₂)ₙ-cycloalkyl; R²² is hydrogen or halogen; R²³ is hydrogen or lower alkyl; R²⁴ is hydrogen, lower alkyl, lower alkinyl, —(CH₂)ₙ—CF₃, —(CH₂)ₙ-cycloalkyl or —(CH₂)ₙ-phenyl optionally substituted by halogen; R²⁵ is hydrogen, lower alkyl, —C(O)H, —C(O)-lower alkyl, —C(O)—CF₃, —C(O)—CH₂F, —C(O)—CHF₂, —C(O)— cycloalkyl, —C(O)—(CH₂)ₙ—O-lower alkyl, —C(O)O—(CH₂)ₙ-cycloalkyl, —C(O)-phenyl optionally substituted by one or more substituents selected from the group consisting of halogen and —C(O)O-lower alkyl, or is —S(O)₂-lower alkyl, —S(O)₂—CF₃, —(CH₂)ₙ-cycloalkyl or is —(CH₂)ₙ-phenyl optionally substituted by halogen; n is 0, 1, 2, 3 or 4.

In some embodiments, the GSI is

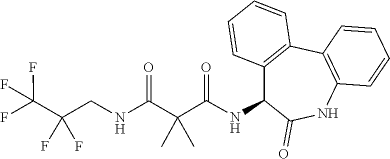 [SMILES: CC(C)(C(=O)NCC(F)(F)C(F)(F)F)C(=O)N[C@@H]1C(=O)Nc2ccccc2-c2ccccc21]

or a pharmaceutically acceptable salt thereof.

In some embodiments, the GSI is a compound described in U.S. Pat. No. 6,984,663. In some embodiments, the GSI is a compound of Formula (V) or a pharmaceutically acceptable salt thereof:

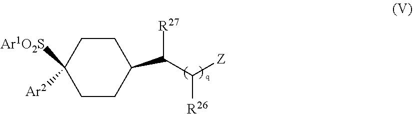

where
q is 0 or 1; Z represents halogen, —CN, —NO₂, —N₃, —CF₃, —OR^{2a}, —N(R^{2a})₂, CO₂R^{2a}, —OCOR^{2a}, —COR^{2a}, —CON(R^{2a})₂, —OCON(R^{2a})₂, —CONR^{2a}(OR^{2a}), —CON(R^{2a})N(R^{2a})₂, —CONHC(═NOH)R^{2a}, heterocyclyl, phenyl or heteroaryl, the heterocyclyl, phenyl or heteroaryl bearing 0-3 substituents selected from halogen, —CN, —NO₂, —CF₃, —OR^{2a}, —N(R^{2a})₂, CO₂R^{2a}, —COR^{2a}, —CON(R^{2a})₂ and C₁₋₄ alkyl; R²⁷ represents H, C₁₋₄ alkyl, or OH; R²⁶ represents H or C₁₋₄ alkyl; with the proviso that when m is 1, R²⁶ and R²⁷ do not both represent C₁₋₄ alkyl; Ar¹ represents C₆₋₁₀ aryl or heteroaryl, either of which bears 0-3 substituents independently selected from halogen, —CN, —NO₂, —CF₃, —OH, —OCF₃, C₁₋₄ alkoxy or C₁₋₄ alkyl which optionally bears a substituent selected from halogen, CN, NO₂, OF₃, OH and C₁₋₄ alkoxy; Ar² represents C₆₋₁₀ aryl or heteroaryl, either of which bears 0-3 substituents independently selected from halogen, —CN, —NO₂, —CF₃, —OH, —OCF₃, C₁₋₄ alkoxy or C₁₋₄ alkyl which optionally bears a substituent selected from halogen, —CN, —NO₂, —CF₃, —OH and C₁₋₄ alkoxy; R^{2a} represents H, C₁₋₆ alkyl, C₃₋₆ cycloalkyl, C₃₋₆ cycloalkyl, C₁₋₆ alkyl, C₂₋₆ alkenyl, any of which optionally bears a substituent selected from halogen, —CN, —NO₂, —CF₃, —OR^{2b}, —CO₂R^{2b}, —N(R^{2b})₂, CON(R²)₂, Ar and COAr; or R^{2a} represents Ar; or two R^{2a} groups together with a nitrogen atom to which they are mutually attached may complete an N-heterocyclyl group bearing 0-4 substituents independently selected from ═O, ═S, halogen, C₁₋₄ alkyl, —CN, —NO₂, —CF₃, —OH, C₁₋₄ alkoxy, C₁₋₄ alkoxycarbonyl, CO₂H, amino, C₁₋₄ alkylamino, di(C₁₋₄alkyl)amino, carbamoyl, Ar and COAr; R^{2b} represents H, C₁₋₆ alkyl, C₃₋₆ cycloalkyl, C₃₋₆ cycloalkylC₁₋₆ alkyl, C₂₋₆ alkenyl, any of which optionally bears a substituent selected from halogen, —CN, —NO₂, —CF₃, —OH, C₁₋₄ alkoxy, C₁₋₄ alkoxycarbonyl, —CO₂H, amino, C₁₋₄ alkylamino, di(C₁₋₄alkyl)amino, carbamoyl, Ar and COAr; or R^{2b} represents Ar; or two R^{2b} groups together with a nitrogen atom to which they are mutually attached may complete an N-heterocyclyl group bearing 0-4 substituents independently selected from ═O, ═S, halogen, C₁₋₄ alkyl, —CN, —NO₂, CF₃, —OH, C₁₋₄ alkoxy, C₁₋₄ alkoxycarbonyl, —CO₂H, amino, C₁₋₄ alkylamino, di(C₁₋₄alkyl)amino, carbamoyl, Ar and COAr; Ar represents phenyl or heteroaryl bearing 0-3 substituents selected from halogen, C₁₋₄ alkyl, —CN, —NO₂, —CF₃, —OH, C₁₋₄ alkoxy, C₁₋₄ alkoxycarbonyl, amino, C₁₋₄ alkylamino, di(C₁₋₄ alkyl)amino, carbamoyl, C₁₋₄ alkylcarbamoyl and di(C₁₋₄ alkyl)carbamoyl.

In some embodiments, the GSI is

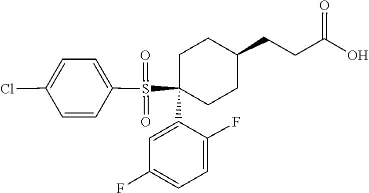 [SMILES: O=C(O)CC[C@H]1CC[C@@](c2cc(F)ccc2F)(S(=O)(=O)c2ccc(Cl)cc2)CC1]

or a pharmaceutically acceptable salt thereof.

In some embodiments, the GSI is a compound described in U.S. Pat. No. 7,795,447. In some embodiments, the GSI is a compound of formula (VI) or a pharmaceutically acceptable salt thereof.

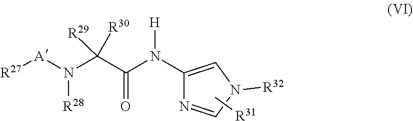

where A′ is absent or selected from

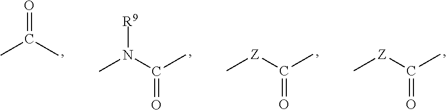

### and —S(O)₂—;

Z is selected from —CH₂, —CH(OH), —CH(C₁-C₆ alkyl), —CH(C₁-C₆ alkoxy), —CH(NR³³R³⁴), —CH(CH₂(OH)), —CH(CH(C₁-C₄ alkyl)(OH)) and —CH(C(C₁-C₄ alkyl)(C₁-C₄ alkyl)(OH)), for example —CH(C(CH₃)(CH₃)(OH)) or —CH(C(CH₃)(CH₂CH₃)(OH)); R²⁷ is selected from C₁-C₂₀ alkyl, C₂-C₂₀ alkenyl, C₂-C₂₀ alkynyl, C₁-C₂₀ alkoxy, C₂-C₂₀ alkenoxy, C₁-C₂₀ hydroxyalkyl, C₃-C₈ cycloalkyl, benzo(C₃-C₈ cycloalkyl), benzo(C₃-C₈ heterocycloalkyl), C₄-C₈ cycloalkenyl, (C₅-C₁₁)bi- or tricycloalkyl, benzo(C₅-C₁₁)bi- or tricycloalkyl, C₇-C₁₁tricycloalkenyl, (3-8 membered) heterocycloalkyl, C₆-C₁₄ aryl and (5-14 membered) heteroaryl, where each hydrogen atom of the alkyl, alkenyl, alkynyl, alkoxy and alkenoxy is optionally independently replaced with halo, and where the cycloalkyl, benzo(C₃-C₈ cycloalkyl), cycloalkenyl, (3-8 membered) heterocycloalkyl, C₆-C₁₄ aryl and (5-14 membered) heteroaryl is optionally independently substituted with from one to four substituents independently selected from C₁-C₁₀ alkyl optionally substituted with from one to three halo atoms, C₁-C₁₀ alkoxy optionally substituted with from one to three halo atoms, C₁-C₁₀ hydroxyalkyl, halo, e.g., fluorine, —OH, —CN,—NR³³R³⁴, —C(═O)NR³³R³⁴, —C(═O)R³⁵, C₃-C₈ cycloalkyl and (3-8 membered) heterocycloalkyl; R²⁸ is selected from H, C₁-C₆ alkyl, C₂-C₆ alkenyl, C₃-C₈ cycloalkyl and C₅-C₈ cycloalkenyl, where R²⁸ is optionally independently substituted with from one to three substituents independently selected from C₁-C₄ alkyl optionally substituted with from one to three halo atoms, C₁-C₄ alkoxy optionally substituted with from one to three halo atoms, halo and —OH; or R²⁷ and R²⁸ together with the A′ group when present and the nitrogen atom to which R2 is attached, or R1 and R2 together with the nitrogen atom to which R²⁷ and R²⁸ are attached when A is absent, may optionally form a four to eight membered ring; R²⁹ is selected from H, C₁-C₆ alkyl, C₂-C₆ alkenyl, C₂-C₆ alkynyl, C₃-C₆ cycloalkyl, C₅-C₆ cycloalkenyl and (3-8 membered) heterocycloalkyl, where the alkyl, alkenyl, alkynyl, cycloalkyl, cycloalkenyl and heterocycloalkyl are each optionally independently substituted with from one to three substituents independently selected from C₁-C₄alkoxy, halo, —OH—S(C₁-C₄)alkyl and (3-8 membered) heterocycloalkyl; R³⁰ is H, C₁-C₆ alkyl or halo; or R3 and R4 may together with the carbon atom to which they are attached optionally form a moiety selected from cyclopropyl, cyclobutyl, cyclopentyl, cyclohexyl, morpholino, piperidino, pyrrolidino, tetrahydrofuranyl and perhydro-2H-pyran, where the moiety formed by R²⁹ and R³⁰ is optionally substituted with from one to three substituents independently selected from C₁-C₆ alkyl optionally substituted with from one to three halo atoms, C₁-C₆ alkoxy optionally substituted with from one to three halo atoms, halo, —OH, —CN and allyl; R³¹ is selected from H, C₁-C₆ alkyl, C₂-C₆ alkylene, C₁-C₆ alkoxy, halo, —CN, C₃-C₁₂ cycloalkyl, C₄-C₁₁ cycloalkenyl and C₆-C₁₀ aryl, (5-10 membered) heteroaryl, where the alkyl, alkylene and alkoxy of R³¹ are each optionally independently substituted with from one to three substituents independently selected from halo and —CN, and where the cycloalkyl, cycloalkenyl and aryl and heteroaryl of R³¹ are each optionally independently substituted with from one to three substituents independently selected from C₁-C₄ alkyl optionally substituted with from one to three halo atoms, C₁-C₄ alkoxy optionally substituted with from one to three halo atoms, halo and —CN; R³² is selected from H, C₁-C₂₀ alkyl, C₁-C₂₀ alkoxy, C₁-C₂₀ hydroxyalkyl, C₃-C₁₂ cycloalkyl, C₄-C₁₂ cycloalkenyl, (C₅-C₂₀) bi- or tricycloalkyl, (C₇-C₂₀)bi- or tricycloalkenyl, (3-12 membered) heterocycloalkyl, (7-20 membered) hetero bi- or heterotricycloalkyl, C₆-C₁₄ aryl and (5-15 membered) heteroaryl, where R³² is optionally independently substituted with from one to four substituents independently selected from C₁-C₂₀ alkyl optionally substituted with from one to three halo atoms, C₁-C₂₀ alkoxy, —OH, —CN, —NO₂, —NR³³R³⁴, —C(═O)NR³³R³⁴, —C(═O)R³⁵, —C(═O)OR³⁵, —S(O)ₙNR³³R³⁴, —S(O)ₙR³⁵, C₃-C₁₂ cycloalkyl, (4-12 membered) heterocycloalkyl optionally substituted with from one to three OH or halo groups, (4-12 membered) heterocycloalkoxy, C₆-C₁₄ aryl, (5-15 membered) heteroaryl, C₆-C₁₂ aryloxy and (5-12 membered) heteroaryloxy; or R6 and R7 may together with the carbon and nitrogen atoms to which they are respectively attached optionally form a (5-8 membered) heterocycloalkyl ring, a (5-8 membered) heterocycloalkenyl ring or a (6-10 membered) heteroaryl ring, where the heterocycloalkyl, heterocycloalkenyl and heteroaryl rings are each optionally independently substituted with from one to three substituents independently selected from halo, C₁-C₆ alkyl, optionally substituted with from one to three halo atoms, C₁-C₆ alkoxy optionally substituted with from one to three halo atoms, C₁-C₆ hydroxyalkyl, —OH, —(CH₂)_{zero-10}NR³³R³⁴, —(CH₂)_{zero-10}C(═O)NR³³R³⁴, —S(O)₂NR³³R³⁴ and C₃-C₁₂ cycloalkyl; R³³ and R³⁴ are each independently selected from H, C₁-C₁₀ alkyl where each hydrogen atom of the C₁-C₁₀ alkyl is optionally independently replaced with a halo atom, e.g., a fluorine atom, C₂-C₁₀ alkenyl, C₂-C₁₀ alkynyl, C₁-C₆ alkoxy where each hydrogen atom of the C₁-C₆ alkoxy is optionally independently replaced with a halo atom, C₂-C₆ alkenoxy, C2-C6 alkynoxy, —C(═O)R11, —S(O)nR11, C₃-C₈ cycloalkyl, C₄-C₈ cycloalkenyl, (C₅-C₁₁)bi- or tricycloalkyl, (C₇-C₁₁)bi- or tricycloalkenyl, (3-8 membered) heterocycloalkyl, C₆-C₁₄ aryl and (5-14 membered) heteroaryl, where the alkyl and alkoxy are each optionally independently substituted with from one to three substituents independently selected from halo and —OH, and where the cycloalkyl, cycloalkenyl, bi- or tricycloalkyl, bi- or tricycloalkenyl, heterocycloalkyl, aryl and heteroaryl are each optionally independently substituted with from one to three substituents independently selected from halo, —OH, C₁-C₆ alkyl optionally independently substituted with from one to six halo atoms, C₂-C₆ alkenyl, C₂-C₆ alkynyl, C₁-C₆ alkoxy, C₂—O₆ alkenoxy, C₂-C₆ alkynoxy and C₁-C₆ hydroxyalkyl; or NR³³R³⁴ may form a (4-7 membered) heterocycloalkyl, where the heterocycloalkyl optionally comprises from one to two further heteroatoms independently selected from N, O, and S, and where the heterocycloalkyl optionally contains from one to three double bonds, and where the heterocycloalkyl is optionally independently substituted with from one to three substituents independently selected from C₁-C₆ alkyl optionally substituted with from one to six halo atoms, C₂-C₆ alkenyl, C₂-C₆ alkynyl, C₁-C₆ alkoxy, C₂—O₆ alkenoxy, C₂-C₆ alkynoxy, C₁-C₆ hydroxyalkyl, C₂-C₆hydroxyalkenyl, C₂-C₆hydroxyalkynyl, halo, —OH, —CN, —NO₂,
—C(═O)R³⁵, —C(═O)OR³⁵, —S(O)ₙR³⁵ and —S(O)ₙNR³³R³⁴; R³⁵ is selected from H, C₁-C₈ alkyl, C₃-C₈ cycloalkyl, C₄-C₈ cycloalkenyl, (C₆-C₁₁)bi- or tricycloalkyl, —(C₇-C₁₁)bi- or tricycloalkenyl, (3-8 membered) heterocycloalkyl, C₆-C₁₀ aryl and (5-14 membered) heteroaryl, where the alkyl of R³⁵ is optionally independently substituted with from one to three substituents independently selected from —OH, —CN and C₃-C₈ cycloalkyl, and where each hydrogen atom of the alkyl is optionally independently replaced with a halo atom, e.g., a fluorine atom, and where the cycloalkyl, cycloalkenyl, heterocycloalkyl, aryl and hetereoaryl of R³⁵ are each optionally independently substituted with from one to three substituents independently selected from halo, C₁-C₈ alkyl optionally substituted with from one to three halo atoms, —OH, —CN and C₃-C₈cycloalkyl; n is in each instance an integer independently selected from zero, 1, 2 and 3; and the pharmaceutically acceptable salts of such compounds.

In some embodiments, the GSI is

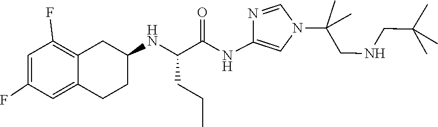 [SMILES: CCC[C@H](N[C@H]1CCc2cc(F)cc(F)c2C1)C(=O)Nc1cn(C(C)(C)CNCC(C)(C)C)cn1]

or a pharmaceutically acceptable salt thereof.

In some embodiments, the GSI is an antibody molecule that reduces the expression and/or function of gamma secretase. In some embodiments, the GSI is an antibody molecule targeting a subunit of gamma secretase. In some embodiments, the GSI is chosen from an anti-presenilin antibody molecule, an anti-nicastrin antibody molecule, an anti-APH-1 antibody molecule, or an anti-PEN-2 antibody molecule.

Exemplary antibody molecules that target a subunit of gamma secretase (e.g., e.g., presenilin, nicastrin, APH-1, or PEN-2) are described in U.S. Pat. Nos. 8,394,376, 8,637,274, and 5,942,400.

Gamma secretase modulators described in WO 2017/019496 can also be used. In some embodiments, the gamma secretase modulator is γ-secretase inhibitor I (GSI I) Z-Leu-Leu-Norleucine; γ-secretase inhibitor II (GSI II); γ-secretase inhibitor III (GSI III), N-Benzyloxycarbonyl-Leu-leucinal, N-(2-Naphthoyl)-Val-phenylalaninal; γ-secretase inhibitor IV (GSI IV); γ-secretase inhibitor V (GSI V), N-Benzyloxycarbonyl-Leu-phenylalaninal; γ-secretase inhibitor VI (GSI VI), 1-(S)-endo-N-(1,3,3)-Trimethylbicyclo[2.2.1]hept-2-yl)-4-fluorophenyl Sulfonamide; γ-secretase inhibitor VII (GSI VII), Menthyloxycarbonyl-LL-CHO; γ-secretase inhibitor IX (GSI IX), (DAPT), N-[N-(3,5-Difluorophenacetyl-L-alanyl)]-S-phenylglycine t-Butyl Ester; γ-secretase inhibitor X (GSI X), {1 S-Benzyl-4R-[1-(1S-carbamoyl-2-phenethylcarbamoyl)-1S-3-methylbutylcarbamoyl]-2R-hydroxy-5-phenylpentyl}carbamic Acid tert-butyl Ester; γ-secretase inhibitor XI (GSI XI), 7-Amino-4-chloro-3-methoxyisocoumarin; γ-secretase inhibitor XII (GSI XII), Z-Ile-Leu-CHO; γ-secretase inhibitor XIII (GSI XIII), Z-Tyr-Ile-Leu-CHO; γ-secretase inhibitor XIV (GSI XIV), Z-Cys(t-Bu)-Ile-Leu-CHO; γ-secretase inhibitor XVI (GSI XVI), N-[N-3,5-Difluorophenacetyl]-L-alanyl-S-phenylglycine Methyl Ester; γ-secretase inhibitor XVII (GSI XVII); γ-secretase inhibitor XIX (GSI XIX), benzo[e][1,4]diazepin-3-yl)-butyramide; γ-secretase inhibitor XX (GSI XX), (S,S)-2-[2-(3,5-Difluorophenyl)acetylamino]-N-(5-methyl-6-oxo-6,7-dihydro-5H-dibenzo[b,d]azepin-7-yl)propionamide; γ-secretase inhibitor XXI (GSI XXI), (S,S)-2-[2-(3,5-Difluorophenyl)-acetylamino]-N-(1-methyl-2-oxo-5-phenyl-2-,3-dihydro-IH-benzo[e][1,4]diazepin-3-yl)-propionamide; Gamma40 secretase inhibitor I, N-trans-3,5-Dimethoxycinnamoyl-Ile-leucinal; Gamma40 secretase inhibitor II, N-tert-Butyloxycarbonyl-Gly-Val-Valinal Isovaleryl-V V-Sta-A-Sta-OCH3; MK-0752 (Merck); MRK-003 (Merck); semagacestat/LY450139 (Eli Lilly); RO4929097; PF-03084014; BMS-708163; MPC-7869 (γ-secretase modifier), YO-01027 (Dibenzazepine); LY411575 (Eli Lilly and Co.); L-685458 (Sigma-Aldrich); BMS-289948 (4-chloro-N-(2,5-difluorophenyl)-N-((IR)-{4-fluoro-2-[3-(1H-imidazol-I-yl)propyl]phenyl}ethyl)benzenesulfonamide hydrochloride); or BMS-299897 (4-[2-((IR)-I-{[(4-chlorophenyl)sulfonyl]-2,5-difluoroanilino}ethyl)-5-fluorophenyljbutanoic acid) (Bristol Myers Squibb).

Exemplary cytokine therapies include interleukin 2 (IL-2) and interferon-alpha (IFN-alpha).

In certain aspects, “cocktails” of different chemotherapeutic agents are administered as the additional agent(s).

In some embodiments, the additional agent(s) to be administered in combination with the CD3 binding molecules are one or more standard of care agents or therapies and/or experimental treatments.

For Hodgkin's lymphoma, combination agents/therapies include radiation and/or chemotherapy (e.g., ABVD (doxorubicin, bleomycin, vinblastine, and dacarbazine), BEACOPP (bleomycin, etoposide, doxorubicin, cyclophosphamide, vincristine, procarbazine and prednisone), or Stanford V (doxorubicin, mechlorethamine (nitrogen mustard), vincristine, vinblastine, bleomycin, etoposide, and prednisone)), antibodies (e.g., brentuximab vedotin, rituximab, or a checkpoint inhibitor such as nivolumab or pembrolizumab).

For DLBCL, combination agents/therapies include monoclonal antibodies (e.g., rituximab (Rituxan)), chemotherapy and/or radiation.

For follicular lymphoma, combination agents/therapies include chemotherapy (e.g., bendamustine (Treanda)); monoclonal antibodies (e.g., rituximab), targeted therapies (e.g., lenalidomide (Revlimid)) and/or radiation.

For mantle cell lymphoma, combination agents/therapies include chemotherapy (including high dose chemotherapy), monoclonal antibodies (e.g., rituximab), targeted therapies (e.g., bortezomib (Velcade), ibrutinib (Imbruvica), and lenalidomide (Revlimid)), stem cell transplants and/or radiation therapy.

For small lymphocytic lymphoma, combination agents/therapies include chemotherapy, monoclonal antibodies, stem cell transplantation, targeted therapies (e.g., ibrutinib), and/or tumor vaccines.

For primary mediastinal large B-cell lymphoma and mediastinal grey-zone lymphoma (MGZL), combination agents/therapies include anthracycline-based chemotherapy, rituximab and/or radiation therapy to the chest.

For splenic marginal zone B-cell lymphoma, combination agents/therapies include the same treatments as follicular lymphoma and additionally in some cases removal of the spleen.

For extranodal marginal zone B-cell lymphoma of MALT, combination agents/therapies include antibiotics (to treat the often causal infection with Helicobacter pylon), radiation therapy, surgery, chemotherapy, and/or monoclonal antibodies.

For nodal marginal zone B-cell lymphoma, combination agents/therapies include the same treatments as follicular lymphoma.

For lymphoplasmacytic lymphoma and the Waldenstrom's macroglobulinemia (WM) variant, combination agents/therapies include those useful for chronic lymphocytic leukemia or follicular lymphoma (see above).

For primary effusion lymphoma, combination agents/therapies include those useful for other diffuse large-cell lymphomas.

For Burkitt lymphoma/Burkitt cell leukemia, combination agents/therapies include intensive chemotherapy.

For multiple myeloma, combination agents/therapies include dexamethasone, pomalidomide (with or without dexamethasone), lenalidomide (with or without dexamethasone), and bortezomib (with or without dexamethasone).

### 8. EXAMPLES

### 8.1. Example 1: Generation of Anti-CD3 Antibodies

Three week to twenty-week old rats were immunized with recombinant human CD3 protein or peptide by a repetitive procedure involving four injections either subcutaneously or interperitoneally. Spleens of immunized rats were harvested, and isolated splenocytes were fused to myeloma cells (P3Ag8.653 cell line) to create hybridoma clones. Supernatant from hybridoma clones was tested with Mirrorball™ (TTPlabtech) as the primary screening assay to identify positive clones binding to human CD3. Supernatants from positive clones identified from the primary screening binding assay against human CD3 were then tested for the ability to bind cynomolgus monkey CD3. Only clones that were able to bind both human CD3 and cyno CD3 are provided.

Methods for primatizing or humanizing non-human antibodies are well known in the art. Generally, a primatized or humanized antibody has one or more amino acid residues introduced into it from a source which is non-primate or non-human. Such non-primate or non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, primatized or humanized antibodies are typically primate or human antibodies in which some complementary determining region (“CDR”) residues and possibly some framework (“FR”) residues are substituted by residues from analogous sites in an originating species (e.g., rodent antibodies) to confer binding specificity.

Alternatively or additionally, an in vivo method for replacing a nonhuman antibody variable region with a human variable region in an antibody while maintaining the same or providing better binding characteristics relative to that of the nonhuman antibody may be utilized to convert non-human antibodies into engineered human antibodies. See, e.g., U.S. Patent Publication No. 2005/0008625, U.S. Patent Publication No. 2005/0255552. Alternatively, human V segment libraries can be generated by sequential cassette replacement in which only part of the reference antibody V segment is initially replaced by a library of human sequences; and identified human “cassettes” supporting binding in the context of residual reference antibody amino acid sequences are then recombined in a second library screen to generate completely human V segments (see, U.S. Patent Publication No. 2006/0134098).

TABLE 19
CD3 binding Sequences

| | | |
|---|---|---|
| NOV292 HC | | |
| SEQ ID NO: 133 | HCDR1 | GFTFSKNGMH |
| (Combined) | | |
| SEQ ID NO: 134 | HCDR2 | MIYYDSSKMYYADTVKG |
| (Combined) | | |
| SEQ ID NO: 135 | HCDR3 | FVWVDLDFDH |
| (Combined) | | |
| SEQ ID NO: 136 | HCDR1 | KNGMH |
| (Kabat) | | |
| SEQ ID NO: 137 | HCDR2 | MIYYDSSKMYYADTVKG |
| (Kabat) | | |
| SEQ ID NO: 138 | HCDR3 | FVWVDLDFDH |
| (Kabat) | | |
| SEQ ID NO: 139 | HCDR1 | GFTFSKN |
| (Chothia) | | |
| SEQ ID NO: 140 | HCDR2 | YYDSSK |
| (Chothia) | | |
| SEQ ID NO: 141 | HCDR3 | FVWVDLDFDH |
| (Chothia) | | |
| SEQ ID NO: 142 | HCDR1 | GFTFSKNG |
| (IMGT) | | |
| SEQ ID NO: 143 | HCDR2 | IYYDSSKM |
| (IMGT) | | |
| SEQ ID NO: 144 | HCDR3 | ASFVWVDLDFDH |
| (IMGT) | | |
| SEQ ID NO: 145 | VH | QVQLVESGGGVVQPGRSLRLSCAASGFTFSKNGMHVVVRQAPGK |
| | | GLEWVAMIYYDSSKMYYADTVKGRFTISRDNSKNTLYLQMNSLRA |
| | | EDTAVYYCASFVWVDLDFDHWGQGTMVTVSS |
| SEQ ID NO: 146 | DNA VH | CAGGTGCAGCTGGTGGAGTCTGGAGGAGGAGTGGTGCAGCCA |
| | | GGCCGGTCCCTGAGACTGTCTTGCGCCGCCAGCGGCTTCACC |
| | | TTTAGCAAGAACGGAATGCACTGGGTGCGGCAGGCACCTGGC |
| | | AAGGGACTGGAGTGGGTGGCCATGATCTACTATGATAGCTCCA |
| | | AGATGTACTATGCCGACACCGTGAAGGGCAGGTTCACAATCTC |
| | | CCGCGATAACTCTAAGAATACCCTGTACCTGCAGATGAATAGC |
| | | CTGAGGGCCGAGGACACAGCCGTGTACTATTGTGCCTCCTTCT |
| | | GGTGGGACCTGGATTTTGACCACTGGGGCCAGGGCACCATGG |
| | | TGACAGTGTCTAGC |
| SEQ ID NO: 147 | Heavy | QVQLVESGGGVVQPGRSLRLSCAASGFTFSKNGMHWVRQAPGK |
| | Chain | GLEWVAMIYYDSSKMYYADTVKGRFTISRDNSKNTLYLQMNSLRA |
| | | EDTAVYYCASFVWVDLDFDHWGQGTMVTVSSASTKGPSVFPLAP |
| | | SSKSTSGGTAALGCLVKDYFPEPVTVSVVNSGALTSGVHTFPAVLQ |
| | | SSGLYSLSSVVTVPSSSLGTQTYICNVNHKPSNTKVDKRVEPKSC |
| | | DKTHTCPPCPAPEAAGGPSVFLFPPKPKDTLMISRTPEVTCVVVD |
| | | VSHEDPEVKFNWYVDGVEVHNAKTKPREEQYNSTYRVVSVLTVL |
| | | HQDWLNGKEYKCKVSNKALPAPIEKTISKAKGQPREPQVYTLPPS |
| | | REEMTKNQVSLTCLVKGFYPSDIAVEVVESNGQPENNYKTTPPVLD |
| | | SDGSFFLYSKLTVDKSRWQQGNVFSCSVMHEALHNHYTQKSLSL |
| | | SPGK |
| SEQ ID NO: 148 | DNA | CAGGTGCAGCTGGTGGAGTCTGGAGGAGGAGTGGTGCAGCCA |
| | Heavy | GGCCGGTCCCTGAGACTGTCTTGCGCCGCCAGCGGCTTCACC |
| | Chain | TTTAGCAAGAACGGAATGCACTGGGTGCGGCAGGCACCTGGC |
| | | AAGGGACTGGAGTGGGTGGCCATGATCTACTATGATAGCTCCA |
| | | AGATGTACTATGCCGACACCGTGAAGGGCAGGTTCACAATCTC |
| | | CCGCGATAACTCTAAGAATACCCTGTACCTGCAGATGAATAGC |
| | | CTGAGGGCCGAGGACACAGCCGTGTACTATTGTGCCTCCTTCT |
| | | GGTGGGACCTGGATTTTGACCACTGGGGCCAGGGCACCATGG |
| | | TGACAGTGTCTAGCGCTAGCACCAAGGGCCCATCGGTCTTCCC |
| | | CCTGGCACCCTCCTCCAAGAGCACCTCTGGGGGCACAGCGGC |
| | | CCTGGGCTGCCTGGTCAAGGACTACTTCCCCGAACCGGTGAC |
| | | GGTGTCGTGGAACTCAGGCGCCCTGACCAGCGGCGTGCACAC |
| | | CTTCCCGGCTGTCCTACAGTCCTCAGGACTCTACTCCCTCAGC |
| | | AGCGTGGTGACCGTGCCCTCCAGCAGCTTGGGCACCCAGACC |
| | | TACATCTGCAACGTGAATCACAAGCCCAGCAACACCAAGGTGG |
| | | ACAAGAGAGTTGAGCCCAAATCTTGTGACAAAACTCACACATGC |
| | | CCACCGTGCCCAGCACCTGAAGCCGCTGGGGGACCGTCAGTC |
| | | TTCCTCTTCCCCCCAAAACCCAAGGACACCCTCATGATCTCCC |
| | | GGACCCCTGAGGTCACATGCGTGGTGGTGGACGTGAGCCACG |
| | | AAGACCCTGAGGTCAAGTTCAACTGGTACGTGGACGGCGTGGA |
| | | GGTGCATAATGCCAAGACAAAGCCGCGGGAGGAGCAGTACAA |
| | | CAGCACGTACCGTGTGGTCAGCGTCCTCACCGTCCTGCACCAG |
| | | GACTGGCTGAATGGCAAGGAGTACAAGTGCAAGGTCTCCAACA |
| | | AAGCCCTCCCAGCCCCCATCGAGAAAACCATCTCCAAAGCCAA |
| | | AGGGCAGCCCCGAGAACCACAGGTGTACACCCTGCCCCCATC |
| | | CCGGGAGGAGATGACCAAGAACCAGGTCAGCCTGACCTGCCT |
| | | GGTCAAAGGCTTCTATCCCAGCGACATCGCCGTGGAGTGGGA |
| | | GAGCAATGGGCAGCCGGAGAACAACTACAAGACCACGCCTCC |
| | | CGTGCTGGACTCCGACGGCTCCTTCTTCCTCTACAGCAAGCTC |
| | | ACCGTGGACAAGAGCAGGTGGCAGCAGGGGAACGTCTTCTCA |
| | | TGCTCCGTGATGCATGAGGCTCTGCACAACCACTACACGCAGA |
| | | AGAGCCTCTCCCTGTCTCCGGGTAAA |
| NOV292 LC | | |
| SEQ ID NO: 149 | LCDR1 | RSSQSLVRSDGTTYFN |
| (Combined) | | |
| SEQ ID NO: 150 | LCDR2 | RVSNRFS |
| (Combined) | | |
| SEQ ID NO: 151 | LCDR3 | LQSSHFPVVT |
| (Combined) | | |
| SEQ ID NO: 152 | LCDR1 | RSSQSLVRSDGTTYFN |
| (Kabat) | | |
| SEQ ID NO: 153 | LCDR2 | RVSNRFS |
| (Kabat) | | |
| SEQ ID NO: 154 | LCDR3 | LQSSHFPWT |
| (Kabat) | | |
| SEQ ID NO: 155 | LCDR1 | SQSLVRSDGTTY |
| (Chothia) | | |
| SEQ ID NO: 156 | LCDR2 | RVS |
| (Chothia) | | |
| SEQ ID NO: 157 | LCDR3 | SSHFPW |
| (Chothia) | | |
| SEQ ID NO: 158 | LCDR1 | QSLVRSDGTTY |
| (IMGT) | | |
| SEQ ID NO: 159 | LCDR2 | RVS |
| (IMGT) | | |
| SEQ ID NO: 160 | LCDR3 | LQSSHFPVVT |
| (IMGT) | | |
| SEQ ID NO: 161 | VL | DIVMTQTPLSSPVTLGQPASISCRSSQSLVRSDGTTYFNWYQQRP |
| | | GQPPRLLIYRVSNRFSGVPDRFSGSGAGTDFTLKISRVEAEDVGV |
| | | YYCLQSSHFPVVTFGGGTKVEIK |
| SEQ ID NO: 162 | DNA VL | GACATCGTGATGACCCAGACACCACTGAGCTCCCCAGTGACCC |
| | | TGGGACAGCCAGCCAGCATCTCCTGCCGGTCTAGCCAGTCCCT |
| | | GGTGAGATCTGATGGCACCACATACTTCAACTGGTATCAGCAG |
| | | AGGCCTGGACAGCCACCTAGGCTGCTGATCTACCGGGTGAGC |
| | | AATAGATTCTCCGGCGTGCCAGACAGGTTTTCTGGCAGCGGAG |
| | | CAGGAACCGACTTCACCCTGAAGATCTCTAGAGTGGAGGCCGA |
| | | GGACGTGGGCGTGTACTATTGTCTGCAGTCCTCTCACTTCCCT |
| | | TGGACCTTTGGCGGCGGCACAAAGGTGGAGATCAAG |
| SEQ ID NO: 163 | Light | DIVMTQTPLSSPVTLGQPASISCRSSQSLVRSDGTTYFNWYQQRP |
| | chain | GQPPRLLIYRVSNRFSGVPDRFSGSGAGTDFTLKISRVEAEDVGV |
| | | YYCLQSSHFPVVTFGGGTKVEIKRTVAAPSVFIFPPSDEQLKSGTAS |
| | | VVCLLNNFYPREAKVQVVKVDNALQSGNSQESVTEQDSKDSTYSL |
| | | SSTLTLSKADYEKHKVYACEVTHQGLSSPVTKSFNRGEC |
| SEQ ID NO: 164 | DNA | GACATCGTGATGACCCAGACACCACTGAGCTCCCCAGTGACCC |
| | Light | TGGGACAGCCAGCCAGCATCTCCTGCCGGTCTAGCCAGTCCCT |
| | Chain | GGTGAGATCTGATGGCACCACATACTTCAACTGGTATCAGCAG |
| | | AGGCCTGGACAGCCACCTAGGCTGCTGATCTACCGGGTGAGC |
| | | AATAGATTCTCCGGCGTGCCAGACAGGTTTTCTGGCAGCGGAG |
| | | CAGGAACCGACTTCACCCTGAAGATCTCTAGAGTGGAGGCCGA |
| | | GGACGTGGGCGTGTACTATTGTCTGCAGTCCTCTCACTTCCCT |
| | | TGGACCTTTGGCGGCGGCACAAAGGTGGAGATCAAGCGTACG |
| | | GTGGCTGCACCATCTGTCTTCATCTTCCCGCCATCTGATGAGC |
| | | AGTTGAAATCTGGAACTGCCTCTGTTGTGTGCCTGCTGAATAAC |
| | | TTCTATCCCCGCGAGGCCAAAGTACAGTGGAAGGTGGATAACG |
| | | CCCTCCAATCGGGTAACTCCCAGGAGAGTGTCACAGAGCAGGA |
| | | CAGCAAGGACAGCACCTACAGCCTCAGCAGCACCCTGACGCT |
| | | GAGCAAAGCAGACTACGAGAAACACAAAGTCTACGCCTGCGAA |
| | | GTCACCCATCAGGGCCTGAGCTCGCCCGTCACAAAGAGCTTCA |
| | | ACCGCGGAGAGTGT |
| NOV123 HC | | |
| SEQ ID NO: 165 | HCDR1 | GYTFTSYYIY |
| (Combined) | | |
| SEQ ID NO: 166 | HCDR2 | YIYPGHDAIYYSENFKG |
| (Combined) | | |
| SEQ ID NO: 167 | HCDR3 | PNTMMAPLAY |
| (Combined) | | |
| SEQ ID NO: 168 | HCDR1 | SYYIY |
| (Kabat) | | |
| SEQ ID NO: 169 | HCDR2 | YIYPGHDAIYYSENFKG |
| (Kabat) | | |
| SEQ ID NO: 170 | HCDR3 | PNTMMAPLAY |
| (Kabat) | | |
| SEQ ID NO: 171 | HCDR1 | GYTFTSY |
| (Chothia) | | |
| SEQ ID NO: 172 | HCDR2 | YPGHDA |
| (Chothia) | | |
| SEQ ID NO: 173 | HCDR3 | PNTMMAPLAY |
| (Chothia) | | |
| SEQ ID NO: 174 | HCDR1 | GYTFTSYY |
| (IMGT) | | |
| SEQ ID NO: 175 | HCDR2 | IYPGHDAI |
| (IMGT) | | |
| SEQ ID NO: 176 | HCDR3 | VRPNTMMAPLAY |
| (IMGT) | | |
| SEQ ID NO: 177 | VH | QVQLVQSGAEVKKPGASVKVSCKASGYTFTSYYIYWVRQAPGQR |
| | | LEWMGYIYPGHDAIYYSENFKGRVTITADTSASTAYMELSSLRSED |
| | | TAVYYCVRPNTMMAPLAYWGQGTLVTVSS |
| SEQ ID NO: 178 | DNA VH | CAGGTGCAGCTGGTGCAGAGCGGAGCAGAGGTGAAGAAGCCA |
| | | GGAGCCAGCGTGAAGGTGTCCTGCAAGGCCTCTGGCTACACC |
| | | TTCACATCCTACTATATCTATTGGGTGCGGCAGGCACCAGGAC |
| | | AGAGACTGGAGTGGATGGGCTACATCTATCCCGGCCACGACG |
| | | CCATCTACTATTCTGAGAACTTTAAGGGCCGGGTGACCATCACA |
| | | GCCGATACCTCCGCCTCTACAGCCTACATGGAGCTGAGCTCCC |
| | | TGAGGAGCGAGGACACCGCCGTGTACTATTGCGTGCGGCCCA |
| | | ATACAATGATGGCACCTCTGGCCTATTGGGGACAGGGCACCCT |
| | | GGTGACAGTGTCTAGC |
| SEQ ID NO: 179 | Heavy | QVQLVQSGAEVKKPGASVKVSCKASGYTFTSYYIYWVRQAPGQR |
| | Chain | LEWMGYIYPGHDAIYYSENFKGRVTITADTSASTAYMELSSLRSED |
| | | TAVYYCVRPNTMMAPLAYWGQGTLVTVSSASTKGPSVFPLAPSS |
| | | KSTSGGTAALGCLVKDYFPEPVTVSVVNSGALTSGVHTFPAVLQSS |
| | | GLYSLSSVVTVPSSSLGTQTYICNVNHKPSNTKVDKRVEPKSCDK |
| | | THTCPPCPAPEAAGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKALPAPIEKTISKAKGQPREPQVYTLPPSRE |
| | | EMTKNQVSLTCLVKGFYPSDIAVEVVESNGQPENNYKTTPPVLDSD |
| | | GSFFLYSKLTVDKSRWQQGNVFSCSVMHEALHNHYTQKSLSLSP |
| | | GK |
| SEQ ID NO: 180 | DNA | CAGGTGCAGCTGGTGCAGAGCGGAGCAGAGGTGAAGAAGCC |
| | Heavy | AGGAGCCAGCGTGAAGGTGTCCTGCAAGGCCTCTGGCTACAC |
| | Chain | CTTCACATCCTACTATATCTATTGGGTGCGGCAGGCACCAGGA |
| | | CAGAGACTGGAGTGGATGGGCTACATCTATCCCGGCCACGAC |
| | | GCCATCTACTATTCTGAGAACTTTAAGGGCCGGGTGACCATCA |
| | | CAGCCGATACCTCCGCCTCTACAGCCTACATGGAGCTGAGCTC |
| | | CCTGAGGAGCGAGGACACCGCCGTGTACTATTGCGTGCGGCC |
| | | CAATACAATGATGGCACCTCTGGCCTATTGGGGACAGGGCACC |
| | | CTGGTGACAGTGTCTAGCGCTAGCACCAAGGGCCCATCGGTCT |
| | | TCCCCCTGGCACCCTCCTCCAAGAGCACCTCTGGGGGCACAG |
| | | CGGCCCTGGGCTGCCTGGTCAAGGACTACTTCCCCGAACCGG |
| | | TGACGGTGTCGTGGAACTCAGGCGCCCTGACCAGCGGCGTGC |
| | | ACACCTTCCCGGCTGTCCTACAGTCCTCAGGACTCTACTCCCT |
| | | CAGCAGCGTGGTGACCGTGCCCTCCAGCAGCTTGGGCACCCA |
| | | GACCTACATCTGCAACGTGAATCACAAGCCCAGCAACACCAAG |
| | | GTGGACAAGAGAGTTGAGCCCAAATCTTGTGACAAAACTCACA |
| | | CATGCCCACCGTGCCCAGCACCTGAAGCCGCTGGGGGACCGT |
| | | CAGTCTTCCTCTTCCCCCCAAAACCCAAGGACACCCTCATGATC |
| | | TCCCGGACCCCTGAGGTCACATGCGTGGTGGTGGACGTGAGC |
| | | CACGAAGACCCTGAGGTCAAGTTCAACTGGTACGTGGACGGC |
| | | GTGGAGGTGCATAATGCCAAGACAAAGCCGCGGGAGGAGCAG |
| | | TACAACAGCACGTACCGTGTGGTCAGCGTCCTCACCGTCCTGC |
| | | ACCAGGACTGGCTGAATGGCAAGGAGTACAAGTGCAAGGTCTC |
| | | CAACAAAGCCCTCCCAGCCCCCATCGAGAAAACCATCTCCAAA |
| | | GCCAAAGGGCAGCCCCGAGAACCACAGGTGTACACCCTGCCC |
| | | CCATCCCGGGAGGAGATGACCAAGAACCAGGTCAGCCTGACC |
| | | TGCCTGGTCAAAGGCTTCTATCCCAGCGACATCGCCGTGGAGT |
| | | GGGAGAGCAATGGGCAGCCGGAGAACAACTACAAGACCACGC |
| | | CTCCCGTGCTGGACTCCGACGGCTCCTTCTTCCTCTACAGCAA |
| | | GCTCACCGTGGACAAGAGCAGGTGGCAGCAGGGGAACGTCTT |
| | | CTCATGCTCCGTGATGCATGAGGCTCTGCACAACCACTACACG |
| | | CAGAAGAGCCTCTCCCTGTCTCCGGGTAAA |
| NOV123 LC | | |
| SEQ ID NO: 181 | LCDR1 | RSSQSLIYSIGNTYLH |
| (Combined) | | |
| SEQ ID NO: 182 | LCDR2 | RVSNRFS |
| (Combined) | | |
| SEQ ID NO: 183 | LCDR3 | FQSTHLPYT |
| (Combined) | | |
| SEQ ID NO: 184 | LCDR1 | RSSQSLIYSIGNTYLH |
| (Kabat) | | |
| SEQ ID NO: 185 | LCDR2 | RVSNRFS |
| (Kabat) | | |
| SEQ ID NO: 186 | LCDR3 | FQSTHLPYT |
| (Kabat) | | |
| SEQ ID NO: 187 | LCDR1 | SQSLIYSIGNTY |
| (Chothia) | | |
| SEQ ID NO: 188 | LCDR2 | RVS |
| (Chothia) | | |
| SEQ ID NO: 189 | LCDR3 | STHLPY |
| (Chothia) | | |
| SEQ ID NO: 190 | LCDR1 | QSLIYSIGNTY |
| (IMGT) | | |
| SEQ ID NO: 191 | LCDR2 | RVS |
| (IMGT) | | |
| SEQ ID NO: 192 | LCDR3 | FQSTHLPYT |
| (IMGT) | | |
| SEQ ID NO: 193 | VL | DVVMTQSPLSLPVTLGQPASISCRSSQSLIYSIGNTYLHWYQQRP |
| | | GQSPRLLIYRVSNRFSGVPDRFSGSGSGTDFTLKISRVEAEDVGV |
| | | YYCFQSTHLPYTFGQGTKLEIK |
| SEQ ID NO: 194 | DNA VL | GACGTGGTCATGACACAGTCCCCACTGTCTCTGCCTGTGACCC |
| | | TGGGACAGCCAGCCTCTATCAGCTGCCGGAGCTCCCAGAGCC |
| | | TGATCTACTCCATCGGCAACACATACCTGCACTGGTATCAGCA |
| | | GAGGCCAGGACAGTCCCCAAGGCTGCTGATCTATCGGGTGTCT |
| | | AATAGATTCAGCGGCGTGCCTGACCGGTTTTCCGGCTCTGGCA |
| | | GCGGCACCGATTTCACACTGAAGATCTCCAGGGTGGAGGCCG |
| | | AGGATGTGGGCGTGTACTATTGTTTCCAGTCTACCCACCTGCC |
| | | ATACACATTTGGCCAGGGCACCAAGCTGGAGATCAAG |
| SEQ ID NO: 195 | Light | DVVMTQSPLSLPVTLGQPASISCRSSQSLIYSIGNTYLHWYQQRP |
| | chain | GQSPRLLIYRVSNRFSGVPDRFSGSGSGTDFTLKISRVEAEDVGV |
| | | YYCFQSTHLPYTFGQGTKLEIKRTVAAPSVFIFPPSDEQLKSGTAS |
| | | VVCLLNNFYPREAKVQWKVDNALQSGNSQESVTEQDSKDSTYSL |
| | | SSTLTLSKADYEKHKVYACEVTHQGLSSPVTKSFNRGEC |
| SEQ ID NO: 196 | DNA | GACGTGGTCATGACACAGTCCCCACTGTCTCTGCCTGTGACCC |
| | Light | TGGGACAGCCAGCCTCTATCAGCTGCCGGAGCTCCCAGAGCC |
| | Chain | TGATCTACTCCATCGGCAACACATACCTGCACTGGTATCAGCA |
| | | GAGGCCAGGACAGTCCCCAAGGCTGCTGATCTATCGGGTGTCT |
| | | AATAGATTCAGCGGCGTGCCTGACCGGTTTTCCGGCTCTGGCA |
| | | GCGGCACCGATTTCACACTGAAGATCTCCAGGGTGGAGGCCG |
| | | AGGATGTGGGCGTGTACTATTGTTTCCAGTCTACCCACCTGCC |
| | | ATACACATTTGGCCAGGGCACCAAGCTGGAGATCAAGCGTACG |
| | | GTGGCTGCACCATCTGTCTTCATCTTCCCGCCATCTGATGAGC |
| | | AGTTGAAATCTGGAACTGCCTCTGTTGTGTGCCTGCTGAATAAC |
| | | TTCTATCCCCGCGAGGCCAAAGTACAGTGGAAGGTGGATAACG |
| | | CCCTCCAATCGGGTAACTCCCAGGAGAGTGTCACAGAGCAGGA |
| | | CAGCAAGGACAGCACCTACAGCCTCAGCAGCACCCTGACGCT |
| | | GAGCAAAGCAGACTACGAGAAACACAAAGTCTACGCCTGCGAA |
| | | GTCACCCATCAGGGCCTGAGCTCGCCCGTCACAAAGAGCTTCA |
| | | ACCGCGGAGAGTGT |
| NOV453 HC | | |
| SEQ ID NO: 197 | HCDR1 | GFSLTTYNVH |
| (Combined) | | |
| SEQ ID NO: 198 | HCDR2 | RMRYSGDTSFNAALTS |
| (Combined) | | |
| SEQ ID NO: 199 | HCDR3 | DPMYIPNYSYGVMNA |
| (Combined) | | |
| SEQ ID NO: 200 | HCDR1 | TYNVH |
| (Kabat) | | |
| SEQ ID NO: 201 | HCDR2 | RMRYSGDTSFNAALTS |
| (Kabat) | | |
| SEQ ID NO: 202 | HCDR3 | DPMYIPNYSYGVMNA |
| (Kabat) | | |
| SEQ ID NO: 203 | HCDR1 | GFSLTTY |
| (Chothia) | | |
| SEQ ID NO: 204 | HCDR2 | RYSGD |
| (Chothia) | | |
| SEQ ID NO: 205 | HCDR3 | DPMYIPNYSYGVMNA |
| (Chothia) | | |
| SEQ ID NO: 206 | HCDR1 | GFSLTTYN |
| (IMGT) | | |
| SEQ ID NO: 207 | HCDR2 | MRYSGDT |
| (IMGT) | | |
| SEQ ID NO: 208 | HCDR3 | TSDPMYIPNYSYGVMNA |
| (IMGT) | | |
| SEQ ID NO: 209 | VH | QVQLQESGPGLVKPSETLSLTCTVSGFSLTTYNVHWIRQPPGKGL |
| | | EWIGRMRYSGDTSFNAALTSRVTISRDTSKNQVSLKLSSVTAADT |
| | | AVYYCTSDPMYIPNYSYGVMNAWGQGTTVTVSS |
| SEQ ID NO: 210 | DNA VH | CAGGTGCAGCTGCAGGAGAGCGGACCTGGACTGGTGAAGCCA |
| | | AGCGAGACCCTGTCCCTGACCTGCACAGTGTCCGGCTTCTCTC |
| | | TGACCACATACAACGTGCACTGGATCAGACAGCCACCTGGCAA |
| | | GGGACTGGAGTGGATCGGCCGGATGAGATATTCTGGCGACAC |
| | | CAGCTTTAACGCCGCCCTGACCTCTAGGGTGACAATCTCCCGC |
| | | GATACATCTAAGAATCAGGTGTCCCTGAAGCTGAGCTCCGTGA |
| | | CCGCAGCAGACACAGCCGTGTACTATTGTACCAGCGATCCAAT |
| | | GTACATCCCCAACTACTCCTATGGCGTGATGAATGCCTGGGGC |
| | | CAGGGCACCACAGTGACAGTGTCTAGC |
| SEQ ID NO: 211 | Heavy | QVQLQESGPGLVKPSETLSLTCTVSGFSLTTYNVHWIRQPPGKGL |
| | Chain | EWIGRMRYSGDTSFNAALTSRVTISRDTSKNQVSLKLSSVTAADT |
| | | AVYYCTSDPMYIPNYSYGVMNAWGQGTTVTVSSASTKGPSVFPL |
| | | APSSKSTSGGTAALGCLVKDYFPEPVTVSWNSGALTSGVHTFPAV |
| | | LQSSGLYSLSSVVTVPSSSLGTQTYICNVNHKPSNTKVDKRVEPK |
| | | SCDKTHTCPPCPAPEAAGGPSVFLFPPKPKDTLMISRTPEVTCVV |
| | | VDVSHEDPEVKFNWYVDGVEVHNAKTKPREEQYNSTYRVVSVLT |
| | | VLHQDWLNGKEYKCKVSNKALPAPIEKTISKAKGQPREPQVYTLP |
| | | PSREEMTKNQVSLTCLVKGFYPSDIAVEWESNGQPENNYKTTPPV |
| | | LDSDGSFFLYSKLTVDKSRWQQGNVFSCSVMHEALHNHYTQKSL |
| | | SLSPGK |
| SEQ ID NO: 212 | DNA | CAGGTGCAGCTGCAGGAGAGCGGACCTGGACTGGTGAAGCC |
| | Heavy | AAGCGAGACCCTGTCCCTGACCTGCACAGTGTCCGGCTTCTCT |
| | Chain | CTGACCACATACAACGTGCACTGGATCAGACAGCCACCTGGCA |
| | | AGGGACTGGAGTGGATCGGCCGGATGAGATATTCTGGCGACA |
| | | CCAGCTTTAACGCCGCCCTGACCTCTAGGGTGACAATCTCCCG |
| | | CGATACATCTAAGAATCAGGTGTCCCTGAAGCTGAGCTCCGTG |
| | | ACCGCAGCAGACACAGCCGTGTACTATTGTACCAGCGATCCAA |
| | | TGTACATCCCCAACTACTCCTATGGCGTGATGAATGCCTGGGG |
| | | CCAGGGCACCACAGTGACAGTGTCTAGCGCTAGCACCAAGGG |
| | | CCCATCGGTCTTCCCCCTGGCACCCTCCTCCAAGAGCACCTCT |
| | | GGGGGCACAGCGGCCCTGGGCTGCCTGGTCAAGGACTACTTC |
| | | CCCGAACCGGTGACGGTGTCGTGGAACTCAGGCGCCCTGACC |
| | | AGCGGCGTGCACACCTTCCCGGCTGTCCTACAGTCCTCAGGAC |
| | | TCTACTCCCTCAGCAGCGTGGTGACCGTGCCCTCCAGCAGCTT |
| | | GGGCACCCAGACCTACATCTGCAACGTGAATCACAAGCCCAGC |
| | | AACACCAAGGTGGACAAGAGAGTTGAGCCCAAATCTTGTGACA |
| | | AAACTCACACATGCCCACCGTGCCCAGCACCTGAAGCCGCTGG |
| | | GGGACCGTCAGTCTTCCTCTTCCCCCCAAAACCCAAGGACACC |
| | | CTCATGATCTCCCGGACCCCTGAGGTCACATGCGTGGTGGTGG |
| | | ACGTGAGCCACGAAGACCCTGAGGTCAAGTTCAACTGGTACGT |
| | | GGACGGCGTGGAGGTGCATAATGCCAAGACAAAGCCGCGGGA |
| | | GGAGCAGTACAACAGCACGTACCGTGTGGTCAGCGTCCTCACC |
| | | GTCCTGCACCAGGACTGGCTGAATGGCAAGGAGTACAAGTGCA |
| | | AGGTCTCCAACAAAGCCCTCCCAGCCCCCATCGAGAAAACCAT |
| | | CTCCAAAGCCAAAGGGCAGCCCCGAGAACCACAGGTGTACAC |
| | | CCTGCCCCCATCCCGGGAGGAGATGACCAAGAACCAGGTCAG |
| | | CCTGACCTGCCTGGTCAAAGGCTTCTATCCCAGCGACATCGCC |
| | | GTGGAGTGGGAGAGCAATGGGCAGCCGGAGAACAACTACAAG |
| | | ACCACGCCTCCCGTGCTGGACTCCGACGGCTCCTTCTTCCTCT |
| | | ACAGCAAGCTCACCGTGGACAAGAGCAGGTGGCAGCAGGGGA |
| | | ACGTCTTCTCATGCTCCGTGATGCATGAGGCTCTGCACAACCA |
| | | CTACACGCAGAAGAGCCTCTCCCTGTCTCCGGGTAAA |
| NOV453 LC | | |
| SEQ ID NO: 213 | LCDR1 | KASQNINNYLN |
| (Combined) | | |
| SEQ ID NO: 214 | LCDR2 | NTDHLQA |
| (Combined) | | |
| SEQ ID NO: 215 | LCDR3 | LQHRSRYT |
| (Combined) | | |
| SEQ ID NO: 216 | LCDR1 | KASQNINNYLN |
| (Kabat) | | |
| SEQ ID NO: 217 | LCDR2 | NTDHLQA |
| (Kabat) | | |
| SEQ ID NO: 218 | LCDR3 | LQHRSRYT |
| (Kabat) | | |
| SEQ ID NO: 219 | LCDR1 | SQNINNY |
| (Chothia) | | |
| SEQ ID NO: 220 | LCDR2 | NTD |
| (Chothia) | | |
| SEQ ID NO: 221 | LCDR3 | HRSRY |
| (Chothia) | | |
| SEQ ID NO: 222 | LCDR1 | QNINNY |
| (IMGT) | | |
| SEQ ID NO: 223 | LCDR2 | NTDHLQAGVP |
| (IMGT) | | |
| SEQ ID NO: 224 | LCDR3 | LQHRSRYT |
| (IMGT) | | |
| SEQ ID NO: 225 | VL | DIQMTQSPSSLSASVGDRVTITCKASQNINNYLNWYQQKPGKAPK |
| | | LLIYNTDHLQAGVPSRFSGSGSGTDYTLTISSLQPEDFATYFCLQH |
| | | RSRYTFGPGTKVDIK |
| SEQ ID NO: 226 | DNA VL | GACATCCAGATGACCCAGAGCCCTAGCTCCCTGTCCGCCTCTG |
| | | TGGGCGATAGGGTGACCATCACATGCAAGGCCTCCCAGAACAT |
| | | CAACAATTACCTGAATTGGTATCAGCAGAAGCCCGGCAAGGCC |
| | | CCTAAGCTGCTGATCTACAACACAGACCACCTGCAGGCAGGAG |
| | | TGCCATCTCGGTTCAGCGGCTCCGGCTCTGGAACCGACTATAC |
| | | CCTGACAATCTCTAGCCTGCAGCCAGAGGATTTCGCCACATAC |
| | | TTTTGTCTGCAGCACCGGAGCAGATATACCTTTGGCCCCGGCA |
| | | CAAAGGTGGATA |
| SEQ ID NO: 227 | Light | DIQMTQSPSSLSASVGDRVTITCKASQNINNYLNWYQQKPGKAPK |
| | chain | LLIYNTDHLQAGVPSRFSGSGSGTDYTLTISSLQPEDFATYFCLQH |
| | | RSRYTFGPGTKVDIKRTVAAPSVFIFPPSDEQLKSGTASVVCLLNN |
| | | FYPREAKVQWKVDNALQSGNSQESVTEQDSKDSTYSLSSTLTLS |
| | | KADYEKHKVYACEVTHQGLSSPVTKSFNRGEC |
| SEQ ID NO: 228 | DNA | GACATCCAGATGACCCAGAGCCCTAGCTCCCTGTCCGCCTCTG |
| | Light | TGGGCGATAGGGTGACCATCACATGCAAGGCCTCCCAGAACAT |
| | Chain | CAACAATTACCTGAATTGGTATCAGCAGAAGCCCGGCAAGGCC |
| | | CCTAAGCTGCTGATCTACAACACAGACCACCTGCAGGCAGGAG |
| | | TGCCATCTCGGTTCAGCGGCTCCGGCTCTGGAACCGACTATAC |
| | | CCTGACAATCTCTAGCCTGCAGCCAGAGGATTTCGCCACATAC |
| | | TTTTGTCTGCAGCACCGGAGCAGATATACCTTTGGCCCCGGCA |
| | | CAAAGGTGGATACGTACGGTGGCTGCACCATCTGTCTTCATCT |
| | | TCCCGCCATCTGATGAGCAGTTGAAATCTGGAACTGCCTCTGTT |
| | | GTGTGCCTGCTGAATAACTTCTATCCCCGCGAGGCCAAAGTAC |
| | | AGTGGAAGGTGGATAACGCCCTCCAATCGGGTAACTCCCAGGA |
| | | GAGTGTCACAGAGCAGGACAGCAAGGACAGCACCTACAGCCT |
| | | CAGCAGCACCCTGACGCTGAGCAAAGCAGACTACGAGAAACAC |
| | | AAAGTCTACGCCTGCGAAGTCACCCATCAGGGCCTGAGCTCGC |
| | | CCGTCACAAAGAGCTTCAACCGCGGAGAGTGT |
| NOV229 HC | | |
| SEQ ID NO: 229 | HCDR1 | GFSLTTYNVH |
| (Combined) | | |
| SEQ ID NO: 230 | HCDR2 | RMRYSGDTSFNAALTS |
| (Combined) | | |
| SEQ ID NO: 231 | HCDR3 | DPMYIPNYSYGVMNA |
| (Combined) | | |
| SEQ ID NO: 232 | HCDR1 | TYNVH |
| (Kabat) | | |
| SEQ ID NO: 233 | HCDR2 | RMRYSGDTSFNAALTS |
| (Kabat) | | |
| SEQ ID NO: 234 | HCDR3 | DPMYIPNYSYGVMNA |
| (Kabat) | | |
| SEQ ID NO: 235 | HCDR1 | GFSLTTY |
| (Chothia) | | |
| SEQ ID NO: 236 | HCDR2 | RYSGD |
| (Chothia) | | |
| SEQ ID NO: 237 | HCDR3 | DPMYIPNYSYGVMNA |
| (Chothia) | | |
| SEQ ID NO: 238 | HCDR1 | GFSLTTYN |
| (IMGT) | | |
| SEQ ID NO: 239 | HCDR2 | MRYSGDT |
| (IMGT) | | |
| SEQ ID NO: 240 | HCDR3 | ARDPMYIPNYSYGVMNA |
| (IMGT) | | |
| SEQ ID NO: 241 | VH | QVQLQESGPGLVKPSETLSLTCTVSGFSLTTYNVHWIRQPPGKGL |
| | | EWIGRMRYSGDTSFNAALTSRVTISVDTSKNQFSLKLSSVTAADTA |
| | | VYYCARDPMYIPNYSYGVMNAWGQGTTVTVSS |
| SEQ ID NO: 242 | DNA VH | CAGGTGCAGCTGCAGGAGTCTGGACCTGGACTGGTGAAGCCA |
| | | AGCGAGACCCTGTCCCTGACCTGCACAGTGTCCGGCTTCTCTC |
| | | TGACCACATACAACGTGCACTGGATCAGGCAGCCACCTGGCAA |
| | | GGGACTGGAGTGGATCGGCCGGATGAGATATTCTGGCGACAC |
| | | CAGCTTCAACGCCGCCCTGACCAGCAGGGTGACAATCTCCGTG |
| | | GATACATCTAAGAATCAGTTTTCCCTGAAGCTGAGCTCCGTGAC |
| | | CGCAGCAGACACAGCCGTGTACTATTGTGCCCGGGACCCCAT |
| | | GTACATCCCCAACTACTCCTATGGCGTGATGAATGCCTGGGGC |
| | | CAGGGCACCACAGTGACAGTGTCTAGC |
| SEQ ID NO: 243 | Heavy | QVQLQESGPGLVKPSETLSLTCTVSGFSLTTYNVHWIRQPPGKGL |
| | Chain | EWIGRMRYSGDTSFNAALTSRVTISVDTSKNQFSLKLSSVTAADTA |
| | | VYYCARDPMYIPNYSYGVMNAWGQGTTVTVSSASTKGPSVFPLA |
| | | PSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKS |
| | | CDKTHTCPPCPAPEAAGGPSVFLFPPKPKDTLMISRTPEVTCVVV |
| | | DVSHEDPEVKFNVVYVDGVEVHNAKTKPREEQYNSTYRVVSVLTV |
| | | LHQDWLNGKEYKCKVSNKALPAPIEKTISKAKGQPREPQVYTLPP |
| | | SREEMTKNQVSLTCLVKGFYPSDIAVEVVESNGQPENNYKTTPPVL |
| | | DSDGSFFLYSKLTVDKSRWQQGNVFSCSVMHEALHNHYTQKSLS |
| | | LSPGK |
| SEQ ID NO: 244 | DNA | CAGGTGCAGCTGCAGGAGTCTGGACCTGGACTGGTGAAGCCA |
| | Heavy | AGCGAGACCCTGTCCCTGACCTGCACAGTGTCCGGCTTCTCTC |
| | Chain | TGACCACATACAACGTGCACTGGATCAGGCAGCCACCTGGCAA |
| | | GGGACTGGAGTGGATCGGCCGGATGAGATATTCTGGCGACAC |
| | | CAGCTTCAACGCCGCCCTGACCAGCAGGGTGACAATCTCCGTG |
| | | GATACATCTAAGAATCAGTTTTCCCTGAAGCTGAGCTCCGTGAC |
| | | CGCAGCAGACACAGCCGTGTACTATTGTGCCCGGGACCCCAT |
| | | GTACATCCCCAACTACTCCTATGGCGTGATGAATGCCTGGGGC |
| | | CAGGGCACCACAGTGACAGTGTCTAGCGCTAGCACCAAGGGC |
| | | CCATCGGTCTTCCCCCTGGCACCCTCCTCCAAGAGCACCTCTG |
| | | GGGGCACAGCGGCCCTGGGCTGCCTGGTCAAGGACTACTTCC |
| | | CCGAACCGGTGACGGTGTCGTGGAACTCAGGCGCCCTGACCA |
| | | GCGGCGTGCACACCTTCCCGGCTGTCCTACAGTCCTCAGGACT |
| | | CTACTCCCTCAGCAGCGTGGTGACCGTGCCCTCCAGCAGCTTG |
| | | GGCACCCAGACCTACATCTGCAACGTGAATCACAAGCCCAGCA |
| | | ACACCAAGGTGGACAAGAGAGTTGAGCCCAAATCTTGTGACAA |
| | | AACTCACACATGCCCACCGTGCCCAGCACCTGAAGCCGCTGG |
| | | GGGACCGTCAGTCTTCCTCTTCCCCCCAAAACCCAAGGACACC |
| | | CTCATGATCTCCCGGACCCCTGAGGTCACATGCGTGGTGGTGG |
| | | ACGTGAGCCACGAAGACCCTGAGGTCAAGTTCAACTGGTACGT |
| | | GGACGGCGTGGAGGTGCATAATGCCAAGACAAAGCCGCGGGA |
| | | GGAGCAGTACAACAGCACGTACCGTGTGGTCAGCGTCCTCACC |
| | | GTCCTGCACCAGGACTGGCTGAATGGCAAGGAGTACAAGTGCA |
| | | AGGTCTCCAACAAAGCCCTCCCAGCCCCCATCGAGAAAACCAT |
| | | CTCCAAAGCCAAAGGGCAGCCCCGAGAACCACAGGTGTACAC |
| | | CCTGCCCCCATCCCGGGAGGAGATGACCAAGAACCAGGTCAG |
| | | CCTGACCTGCCTGGTCAAAGGCTTCTATCCCAGCGACATCGCC |
| | | GTGGAGTGGGAGAGCAATGGGCAGCCGGAGAACAACTACAAG |
| | | ACCACGCCTCCCGTGCTGGACTCCGACGGCTCCTTCTTCCTCT |
| | | ACAGCAAGCTCACCGTGGACAAGAGCAGGTGGCAGCAGGGGA |
| | | ACGTCTTCTCATGCTCCGTGATGCATGAGGCTCTGCACAACCA |
| | | CTACACGCAGAAGAGCCTCTCCCTGTCTCCGGGTAAA |
| NOV229 LC | | |
| SEQ ID NO: 245 | LCDR1 | KASQNINNYLN |
| (Combined) | | |
| SEQ ID NO: 246 | LCDR2 | NTDHLQA |
| (Combined) | | |
| SEQ ID NO: 247 | LCDR3 | LQHRSRYT |
| (Combined) | | |
| SEQ ID NO: 248 | LCDR1 | KASQNINNYLN |
| (Kabat) | | |
| SEQ ID NO: 249 | LCDR2 | NTDHLQA |
| (Kabat) | | |
| SEQ ID NO: 250 | LCDR3 | LQHRSRYT |
| (Kabat) | | |
| SEQ ID NO: 251 | LCDR1 | SQNINNY |
| (Chothia) | | |
| SEQ ID NO: 252 | LCDR2 | NTD |
| (Chothia) | | |
| SEQ ID NO: 253 | LCDR3 | HRSRY |
| (Chothia) | | |
| SEQ ID NO: 254 | LCDR1 | QNINNY |
| (IMGT) | | |
| SEQ ID NO: 255 | LCDR2 | NTDHLQAGVP |
| (IMGT) | | |
| SEQ ID NO: 256 | LCDR3 | LQHRSRYT |
| (IMGT) | | |
| SEQ ID NO: 257 | VL | DIQMTQSPSSLSASVGDRVTITCKASQNINNYLNWYQQKPGKAPK |
| | | LLIYNTDHLQAGVPSRFSGSGSGTDFTLTISSLQPEDFATYYCLQH |
| | | RSRYTFGPGTKVDIK |
| SEQ ID NO: 258 | DNA VL | GACATCCAGATGACCCAGAGCCCTAGCTCCCTGTCCGCCTCTG |
| | | TGGGCGATAGGGTGACCATCACATGCAAGGCCTCCCAGAACAT |
| | | CAACAATTACCTGAATTGGTATCAGCAGAAGCCCGGCAAGGCC |
| | | CCTAAGCTGCTGATCTACAACACAGACCACCTGCAGGCAGGAG |
| | | TGCCATCTCGGTTCAGCGGCTCCGGCTCTGGAACCGACTTTAC |
| | | CCTGACAATCTCTAGCCTGCAGCCAGAGGATTTCGCCACATAC |
| | | TATTGTCTGCAGCACCGGAGCAGATATACCTTTGGCCCCGGCA |
| | | CAAAGGTGGATATCAAG |
| SEQ ID NO: 259 | Light | DIQMTQSPSSLSASVGDRVTITCKASQNINNYLNWYQQKPGKAPK |
| | chain | LLIYNTDHLQAGVPSRFSGSGSGTDFTLTISSLQPEDFATYYCLQH |
| | | RSRYTFGPGTKVDIKRTVAAPSVFIFPPSDEQLKSGTASVVCLLNN |
| | | FYPREAKVQWKVDNALQSGNSQESVTEQDSKDSTYSLSSTLTLS |
| | | KADYEKHKVYACEVTHQGLSSPVTKSFNRGEC |
| SEQ ID NO: 260 | DNA | GACATCCAGATGACCCAGAGCCCTAGCTCCCTGTCCGCCTCTG |
| | Light | TGGGCGATAGGGTGACCATCACATGCAAGGCCTCCCAGAACAT |
| | Chain | CAACAATTACCTGAATTGGTATCAGCAGAAGCCCGGCAAGGCC |
| | | CCTAAGCTGCTGATCTACAACACAGACCACCTGCAGGCAGGAG |
| | | TGCCATCTCGGTTCAGCGGCTCCGGCTCTGGAACCGACTTTAC |
| | | CCTGACAATCTCTAGCCTGCAGCCAGAGGATTTCGCCACATAC |
| | | TATTGTCTGCAGCACCGGAGCAGATATACCTTTGGCCCCGGCA |
| | | CAAAGGTGGATATCAAGCGTACGGTGGCTGCACCATCTGTCTT |
| | | CATCTTCCCGCCATCTGATGAGCAGTTGAAATCTGGAACTGCCT |
| | | CTGTTGTGTGCCTGCTGAATAACTTCTATCCCCGCGAGGCCAA |
| | | AGTACAGTGGAAGGTGGATAACGCCCTCCAATCGGGTAACTCC |
| | | CAGGAGAGTGTCACAGAGCAGGACAGCAAGGACAGCACCTAC |
| | | AGCCTCAGCAGCACCCTGACGCTGAGCAAAGCAGACTACGAG |
| | | AAACACAAAGTCTACGCCTGCGAAGTCACCCATCAGGGCCTGA |
| | | GCTCGCCCGTCACAAAGAGCTTCAACCGCGGAGAGTGT |
| NOV110 HC | | |
| SEQ ID NO: 261 | HCDR1 | GYTFTSYYIY |
| (Combined) | | |
| SEQ ID NO: 262 | HCDR2 | YIYPANGGIYYSEKFKG |
| (Combined) | | |
| SEQ ID NO: 263 | HCDR3 | PVTMMAPLVF |
| (Combined) | | |
| SEQ ID NO: 264 | HCDR1 | SYYIY |
| (Kabat) | | |
| SEQ ID NO: 265 | HCDR2 | YIYPANGGIYYSEKFKG |
| (Kabat) | | |
| SEQ ID NO: 266 | HCDR3 | PVTMMAPLVF |
| (Kabat) | | |
| SEQ ID NO: 267 | HCDR1 | GYTFTSY |
| (Chothia) | | |
| SEQ ID NO: 268 | HCDR2 | YPANGG |
| (Chothia) | | |
| SEQ ID NO: 269 | HCDR3 | PVTMMAPLVF |
| (Chothia) | | |
| SEQ ID NO: 270 | HCDR1 | GYTFTSYY |
| (IMGT) | | |
| SEQ ID NO: 271 | HCDR2 | IYPANGGI |
| (IMGT) | | |
| SEQ ID NO: 272 | HCDR3 | ARPVTMMAPLVF |
| (IMGT) | | |
| SEQ ID NO: 273 | VH | QVQLVQSGAEVKKPGASVKVSCKASGYTFTSYYIYWVRQAPGQR |
| | | LEVVMGYIYPANGGIYYSEKFKGRVTITADTSAGTAYMELSSLRSED |
| | | TAVYYCARPVTMMAPLVFWGQGTLVTVSS |
| SEQ ID NO: 274 | DNA VH | CAGGTGCAGCTGGTGCAGAGCGGAGCAGAGGTGAAGAAGCCA |
| | | GGAGCCTCCGTGAAGGTGTCTTGCAAGGCCAGCGGCTACACC |
| | | TTCACATCCTACTATATCTATTGGGTGCGGCAGGCACCAGGAC |
| | | AGAGACTGGAGTGGATGGGCTACATCTATCCCGCCAACGGCG |
| | | GCATCTACTATTCTGAGAAGTTTAAGGGCCGGGTGACCATCAC |
| | | AGCCGACACCTCCGCCGGCACAGCCTACATGGAGCTGAGCTC |
| | | CCTGAGGTCTGAGGATACCGCCGTGTACTATTGTGCCCGCCCC |
| | | GTGACAATGATGGCACCTCTGGTGTTCTGGGGACAGGGCACC |
| | | CTGGTGACAGTGTCTAGC |
| SEQ ID NO: 275 | Heavy | QVQLVQSGAEVKKPGASVKVSCKASGYTFTSYYIYWVRQAPGQR |
| | Chain | LEWMGYIYPANGGIYYSEKFKGRVTITADTSAGTAYMELSSLRSED |
| | | TAVYYCARPVTMMAPLVFWGQGTLVTVSSASTKGPSVFPLAPSS |
| | | KSTSGGTAALGCLVKDYFPEPVTVSVVNSGALTSGVHTFPAVLQSS |
| | | GLYSLSSVVTVPSSSLGTQTYICNVNHKPSNTKVDKRVEPKSCDK |
| | | THTCPPCPAPEAAGGPSVFLFPPKPKDTLMISRTPEVTCVVVDVS |
| | | HEDPEVKFNVVYVDGVEVHNAKTKPREEQYNSTYRVVSVLTVLHQ |
| | | DWLNGKEYKCKVSNKALPAPIEKTISKAKGQPREPQVYTLPPSRE |
| | | EMTKNQVSLTCLVKGFYPSDIAVEWESNGQPENNYKTTPPVLDSD |
| | | GSFFLYSKLTVDKSRWQQGNVFSCSVMHEALHNHYTQKSLSLSP |
| | | GK |
| SEQ ID NO: 276 | DNA | CAGGTGCAGCTGGTGCAGAGCGGAGCAGAGGTGAAGAAGCC |
| | Heavy | AGGAGCCTCCGTGAAGGTGTCTTGCAAGGCCAGCGGCTACAC |
| | Chain | CTTCACATCCTACTATATCTATTGGGTGCGGCAGGCACCAGGA |
| | | CAGAGACTGGAGTGGATGGGCTACATCTATCCCGCCAACGGC |
| | | GGCATCTACTATTCTGAGAAGTTTAAGGGCCGGGTGACCATCA |
| | | CAGCCGACACCTCCGCCGGCACAGCCTACATGGAGCTGAGCT |
| | | CCCTGAGGTCTGAGGATACCGCCGTGTACTATTGTGCCCGCCC |
| | | CGTGACAATGATGGCACCTCTGGTGTTCTGGGGACAGGGCAC |
| | | CCTGGTGACAGTGTCTAGCGCTAGCACCAAGGGCCCATCGGT |
| | | CTTCCCCCTGGCACCCTCCTCCAAGAGCACCTCTGGGGGCACA |
| | | GCGGCCCTGGGCTGCCTGGTCAAGGACTACTTCCCCGAACCG |
| | | GTGACGGTGTCGTGGAACTCAGGCGCCCTGACCAGCGGCGTG |
| | | CACACCTTCCCGGCTGTCCTACAGTCCTCAGGACTCTACTCCC |
| | | TCAGCAGCGTGGTGACCGTGCCCTCCAGCAGCTTGGGCACCC |
| | | AGACCTACATCTGCAACGTGAATCACAAGCCCAGCAACACCAA |
| | | GGTGGACAAGAGAGTTGAGCCCAAATCTTGTGACAAAACTCAC |
| | | ACATGCCCACCGTGCCCAGCACCTGAAGCCGCTGGGGGACCG |
| | | TCAGTCTTCCTCTTCCCCCCAAAACCCAAGGACACCCTCATGAT |
| | | CTCCCGGACCCCTGAGGTCACATGCGTGGTGGTGGACGTGAG |
| | | CCACGAAGACCCTGAGGTCAAGTTCAACTGGTACGTGGACGGC |
| | | GTGGAGGTGCATAATGCCAAGACAAAGCCGCGGGAGGAGCAG |
| | | TACAACAGCACGTACCGTGTGGTCAGCGTCCTCACCGTCCTGC |
| | | ACCAGGACTGGCTGAATGGCAAGGAGTACAAGTGCAAGGTCTC |
| | | CAACAAAGCCCTCCCAGCCCCCATCGAGAAAACCATCTCCAAA |
| | | GCCAAAGGGCAGCCCCGAGAACCACAGGTGTACACCCTGCCC |
| | | CCATCCCGGGAGGAGATGACCAAGAACCAGGTCAGCCTGACC |
| | | TGCCTGGTCAAAGGCTTCTATCCCAGCGACATCGCCGTGGAGT |
| | | GGGAGAGCAATGGGCAGCCGGAGAACAACTACAAGACCACGC |
| | | CTCCCGTGCTGGACTCCGACGGCTCCTTCTTCCTCTACAGCAA |
| | | GCTCACCGTGGACAAGAGCAGGTGGCAGCAGGGGAACGTCTT |
| | | CTCATGCTCCGTGATGCATGAGGCTCTGCACAACCACTACACG |
| | | CAGAAGAGCCTCTCCCTGTCTCCGGGTAAA |
| NOV110 LC | | |
| SEQ ID NO: 277 | LCDR1 | RSSQSLVYSHGNTYLH |
| (Combined) | | |
| SEQ ID NO: 278 | LCDR2 | RVSNRFS |
| (Combined) | | |
| SEQ ID NO: 279 | LCDR3 | FQSTHLPYT |
| (Combined) | | |
| SEQ ID NO: 280 | LCDR1 | RSSQSLVYSHGNTYLH |
| (Kabat) | | |
| SEQ ID NO: 281 | LCDR2 | RVSNRFS |
| (Kabat) | | |
| SEQ ID NO: 282 | LCDR3 | FQSTHLPYT |
| (Kabat) | | |
| SEQ ID NO: 283 | LCDR1 | SQSLVYSHGNTY |
| (Chothia) | | |
| SEQ ID NO: 284 | LCDR2 | RVS |
| (Chothia) | | |
| SEQ ID NO: 285 | LCDR3 | STHLPY |
| (Chothia) | | |
| SEQ ID NO: 286 | LCDR1 | QSLVYSHGNTY |
| (IMGT) | | |
| SEQ ID NO: 287 | LCDR2 | RVS |
| (IMGT) | | |
| SEQ ID NO: 288 | LCDR3 | FQSTHLPYT |
| (IMGT) | | |
| SEQ ID NO: 289 | VL | DVVMTQSPLSLPVTLGQPASISCRSSQSLVYSHGNTYLHWYQQR |
| | | PGQSPRLLIYRVSNRFSGVPDRFSGSGSGTDFTLKISRVEAEDVG |
| | | VYYCFQSTHLPYTFGQGTKLEIK |
| SEQ ID NO: 290 | DNA VL | GACGTGGTCATGACACAGTCCCCACTGTCTCTGCCTGTGACCC |
| | | TGGGACAGCCAGCCTCTATCAGCTGCCGGAGCTCCCAGAGCC |
| | | TGGTGTACTCCCACGGCAACACATACCTGCACTGGTATCAGCA |
| | | GAGGCCAGGACAGTCCCCAAGGCTGCTGATCTATCGGGTGTCT |
| | | AATAGATTCAGCGGCGTGCCTGACCGGTTTTCCGGCTCTGGCA |
| | | GCGGCACCGATTTCACACTGAAGATCTCCAGGGTGGAGGCCG |
| | | AGGATGTGGGCGTGTACTATTGTTTCCAGTCTACCCACCTGCC |
| | | ATACACATTTGGCCAGGGCACCAAGCTGGAGATCAAG |
| SEQ ID NO: 291 | Light | DVVMTQSPLSLPVTLGQPASISCRSSQSLVYSHGNTYLHWYQQR |
| | chain | PGQSPRLLIYRVSNRFSGVPDRFSGSGSGTDFTLKISRVEAEDVG |
| | | VYYCFQSTHLPYTFGQGTKLEIKRTVAAPSVFIFPPSDEQLKSGTA |
| | | SVVCLLNNFYPREAKVQWKVDNALQSGNSQESVTEQDSKDSTYS |
| | | LSSTLTLSKADYEKHKVYACEVTHQGLSSPVTKSFNRGEC |
| SEQ ID NO: 292 | DNA | GACGTGGTCATGACACAGTCCCCACTGTCTCTGCCTGTGACCC |
| | Light | TGGGACAGCCAGCCTCTATCAGCTGCCGGAGCTCCCAGAGCC |
| | Chain | TGGTGTACTCCCACGGCAACACATACCTGCACTGGTATCAGCA |
| | | GAGGCCAGGACAGTCCCCAAGGCTGCTGATCTATCGGGTGTCT |
| | | AATAGATTCAGCGGCGTGCCTGACCGGTTTTCCGGCTCTGGCA |
| | | GCGGCACCGATTTCACACTGAAGATCTCCAGGGTGGAGGCCG |
| | | AGGATGTGGGCGTGTACTATTGTTTCCAGTCTACCCACCTGCC |
| | | ATACACATTTGGCCAGGGCACCAAGCTGGAGATCAAGCGTACG |
| | | GTGGCTGCACCATCTGTCTTCATCTTCCCGCCATCTGATGAGC |
| | | AGTTGAAATCTGGAACTGCCTCTGTTGTGTGCCTGCTGAATAAC |
| | | TTCTATCCCCGCGAGGCCAAAGTACAGTGGAAGGTGGATAACG |
| | | CCCTCCAATCGGGTAACTCCCAGGAGAGTGTCACAGAGCAGGA |
| | | CAGCAAGGACAGCACCTACAGCCTCAGCAGCACCCTGACGCT |
| | | GAGCAAAGCAGACTACGAGAAACACAAAGTCTACGCCTGCGAA |
| | | GTCACCCATCAGGGCCTGAGCTCGCCCGTCACAAAGAGCTTCA |
| | | ACCGCGGAGAGTGT |
| NOV832 HC | | |
| SEQ ID NO: 293 | HCDR1 | GYTFTSYYIY |
| (Combined) | | |
| SEQ ID NO: 294 | HCDR2 | YIYPANGGIYYSEKFKG |
| (Combined) | | |
| SEQ ID NO: 295 | HCDR3 | PVTMMAPLVF |
| (Combined) | | |
| SEQ ID NO: 296 | HCDR1 | SYYIY |
| (Kabat) | | |
| SEQ ID NO: 297 | HCDR2 | YIYPANGGIYYSEKFKG |
| (Kabat) | | |
| SEQ ID NO: 298 | HCDR3 | PVTMMAPLVF |
| (Kabat) | | |
| SEQ ID NO: 299 | HCDR1 | GYTFTSY |
| (Chothia) | | |
| SEQ ID NO: 300 | HCDR2 | YPANGG |
| (Chothia) | | |
| SEQ ID NO: 301 | HCDR3 | PVTMMAPLVF |
| (Chothia) | | |
| SEQ ID NO: 302 | HCDR1 | GYTFTSYY |
| (IMGT) | | |
| SEQ ID NO: 303 | HCDR2 | IYPANGGI |
| (IMGT) | | |
| SEQ ID NO: 304 | HCDR3 | ARPVTMMAPLVF |
| (IMGT) | | |
| SEQ ID NO: 305 | VH | QVQLVQSGAEVKKPGASVKVSCKASGYTFTSYYIYWVRQAPGQR |
| | | LEWMGYIYPANGGIYYSEKFKGRVTITRDTSASTAYMELSSLRSED |
| | | TAVYYCARPVTMMAPLVFWGQGTLVTVSS |
| SEQ ID NO: 306 | DNA VH | CAGGTGCAGCTGGTGCAGAGCGGAGCAGAGGTGAAGAAGCCA |
| | | GGAGCCAGCGTGAAGGTGTCCTGCAAGGCCTCTGGCTACACC |
| | | TTCACATCCTACTATATCTATTGGGTGAGGCAGGCACCAGGAC |
| | | AGCGCCTGGAGTGGATGGGCTACATCTATCCCGCCAACGGCG |
| | | GCATCTACTATTCTGAGAAGTTTAAGGGCCGGGTGACCATCAC |
| | | AAGAGACACCTCCGCCTCTACAGCCTACATGGAGCTGAGCTCC |
| | | CTGCGGAGCGAGGATACCGCCGTGTACTATTGTGCCAGGCCC |
| | | GTGACAATGATGGCACCTCTGGTGTTCTGGGGACAGGGCACC |
| | | CTGGTGACAGTGTCTAGC |
| SEQ ID NO: 307 | Heavy | QVQLVQSGAEVKKPGASVKVSCKASGYTFTSYYIYWVRQAPGQR |
| | Chain | LEWMGYIYPANGGIYYSEKFKGRVTITRDTSASTAYMELSSLRSED |
| | | TAVYYCARPVTMMAPLVFWGQGTLVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDK |
| | | THTCPPCPAPEAAGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKALPAPIEKTISKAKGQPREPQVYTLPPSRE |
| | | EMTKNQVSLTCLVKGFYPSDIAVEWESNGQPENNYKTTPPVLDSD |
| | | GSFFLYSKLTVDKSRWQQGNVFSCSVMHEALHNHYTQKSLSLSP |
| | | GK |
| SEQ ID NO: 308 | DNA | CAGGTGCAGCTGGTGCAGAGCGGAGCAGAGGTGAAGAAGCC |
| | Heavy | AGGAGCCAGCGTGAAGGTGTCCTGCAAGGCCTCTGGCTACAC |
| | Chain | CTTCACATCCTACTATATCTATTGGGTGAGGCAGGCACCAGGA |
| | | CAGCGCCTGGAGTGGATGGGCTACATCTATCCCGCCAACGGC |
| | | GGCATCTACTATTCTGAGAAGTTTAAGGGCCGGGTGACCATCA |
| | | CAAGAGACACCTCCGCCTCTACAGCCTACATGGAGCTGAGCTC |
| | | CCTGCGGAGCGAGGATACCGCCGTGTACTATTGTGCCAGGCC |
| | | CGTGACAATGATGGCACCTCTGGTGTTCTGGGGACAGGGCAC |
| | | CCTGGTGACAGTGTCTAGCGCTAGCACCAAGGGCCCATCGGT |
| | | CTTCCCCCTGGCACCCTCCTCCAAGAGCACCTCTGGGGGCACA |
| | | GCGGCCCTGGGCTGCCTGGTCAAGGACTACTTCCCCGAACCG |
| | | GTGACGGTGTCGTGGAACTCAGGCGCCCTGACCAGCGGCGTG |
| | | CACACCTTCCCGGCTGTCCTACAGTCCTCAGGACTCTACTCCC |
| | | TCAGCAGCGTGGTGACCGTGCCCTCCAGCAGCTTGGGCACCC |
| | | AGACCTACATCTGCAACGTGAATCACAAGCCCAGCAACACCAA |
| | | GGTGGACAAGAGAGTTGAGCCCAAATCTTGTGACAAAACTCAC |
| | | ACATGCCCACCGTGCCCAGCACCTGAAGCCGCTGGGGGACCG |
| | | TCAGTCTTCCTCTTCCCCCCAAAACCCAAGGACACCCTCATGAT |
| | | CTCCCGGACCCCTGAGGTCACATGCGTGGTGGTGGACGTGAG |
| | | CCACGAAGACCCTGAGGTCAAGTTCAACTGGTACGTGGACGGC |
| | | GTGGAGGTGCATAATGCCAAGACAAAGCCGCGGGAGGAGCAG |
| | | TACAACAGCACGTACCGTGTGGTCAGCGTCCTCACCGTCCTGC |
| | | ACCAGGACTGGCTGAATGGCAAGGAGTACAAGTGCAAGGTCTC |
| | | CAACAAAGCCCTCCCAGCCCCCATCGAGAAAACCATCTCCAAA |
| | | GCCAAAGGGCAGCCCCGAGAACCACAGGTGTACACCCTGCCC |
| | | CCATCCCGGGAGGAGATGACCAAGAACCAGGTCAGCCTGACC |
| | | TGCCTGGTCAAAGGCTTCTATCCCAGCGACATCGCCGTGGAGT |
| | | GGGAGAGCAATGGGCAGCCGGAGAACAACTACAAGACCACGC |
| | | CTCCCGTGCTGGACTCCGACGGCTCCTTCTTCCTCTACAGCAA |
| | | GCTCACCGTGGACAAGAGCAGGTGGCAGCAGGGGAACGTCTT |
| | | CTCATGCTCCGTGATGCATGAGGCTCTGCACAACCACTACACG |
| | | CAGAAGAGCCTCTCCCTGTCTCCGGGTAAA |
| NOV832 LC | | |
| SEQ ID NO: 309 | LCDR1 | RSSQSLVYSHGNTYLH |
| (Combined) | | |
| SEQ ID NO: 310 | LCDR2 | RVSNRFS |
| (Combined) | | |
| SEQ ID NO: 311 | LCDR3 | FQSTHLPYT |
| (Combined) | | |
| SEQ ID NO: 312 | LCDR1 | RSSQSLVYSHGNTYLH |
| (Kabat) | | |
| SEQ ID NO: 313 | LCDR2 | RVSNRFS |
| (Kabat) | | |
| SEQ ID NO: 314 | LCDR3 | FQSTHLPYT |
| (Kabat) | | |
| SEQ ID NO: 315 | LCDR1 | SQSLVYSHGNTY |
| (Chothia) | | |
| SEQ ID NO: 316 | LCDR2 | RVS |
| (Chothia) | | |
| SEQ ID NO: 317 | LCDR3 | STHLPY |
| (Chothia) | | |
| SEQ ID NO: 318 | LCDR1 | QSLVYSHGNTY |
| (IMGT) | | |
| SEQ ID NO: 319 | LCDR2 | RVS |
| (IMGT) | | |
| SEQ ID NO: 320 | LCDR3 | FQSTHLPYT |
| (IMGT) | | |
| SEQ ID NO: 321 | VL | DVVMTQSPLSLPVTLGQPASISCRSSQSLVYSHGNTYLHWFQQR |
| | | PGQSPRRLIYRVSNRFSGVPDRFSGSGSGTDFTLKISRVEAEDVG |
| | | VYYCFQSTHLPYTFGQGTKLEIK |
| SEQ ID NO: 322 | DNA VL | GACGTGGTCATGACACAGTCCCCACTGTCTCTGCCTGTGACCC |
| | | TGGGACAGCCAGCCTCTATCAGCTGCAGGAGCTCCCAGAGCC |
| | | TGGTGTACTCCCACGGCAACACATATCTGCACTGGTTCCAGCA |
| | | GAGACCAGGACAGTCCCCACGGAGACTGATCTACAGGGTGTCT |
| | | AATCGCTTCAGCGGCGTGCCTGACCGGTTTTCCGGCTCTGGCA |
| | | GCGGAACCGACTTCACCCTGAAGATCTCCAGAGTGGAGGCCG |
| | | AGGATGTGGGCGTGTACTATTGTTTCCAGTCTACCCACCTGCC |
| | | ATATACATTTGGCCAGGGCACCAAGCTGGAGATCAAG |
| SEQ ID NO: 323 | Light | DVVMTQSPLSLPVTLGQPASISCRSSQSLVYSHGNTYLHWFQQR |
| | chain | PGQSPRRLIYRVSNRFSGVPDRFSGSGSGTDFTLKISRVEAEDVG |
| | | VYYCFQSTHLPYTFGQGTKLEIKRTVAAPSVFIFPPSDEQLKSGTA |
| | | SVVCLLNNFYPREAKVQWKVDNALQSGNSQESVTEQDSKDSTYS |
| | | LSSTLTLSKADYEKHKVYACEVTHQGLSSPVTKSFNRGEC |
| SEQ ID NO: 324 | DNA | GACGTGGTCATGACACAGTCCCCACTGTCTCTGCCTGTGACCC |
| | Light | TGGGACAGCCAGCCTCTATCAGCTGCAGGAGCTCCCAGAGCC |
| | Chain | TGGTGTACTCCCACGGCAACACATATCTGCACTGGTTCCAGCA |
| | | GAGACCAGGACAGTCCCCACGGAGACTGATCTACAGGGTGTCT |
| | | AATCGCTTCAGCGGCGTGCCTGACCGGTTTTCCGGCTCTGGCA |
| | | GCGGAACCGACTTCACCCTGAAGATCTCCAGAGTGGAGGCCG |
| | | AGGATGTGGGCGTGTACTATTGTTTCCAGTCTACCCACCTGCC |
| | | ATATACATTTGGCCAGGGCACCAAGCTGGAGATCAAGCGTACG |
| | | GTGGCTGCACCATCTGTCTTCATCTTCCCGCCATCTGATGAGC |
| | | AGTTGAAATCTGGAACTGCCTCTGTTGTGTGCCTGCTGAATAAC |
| | | TTCTATCCCCGCGAGGCCAAAGTACAGTGGAAGGTGGATAACG |
| | | CCCTCCAATCGGGTAACTCCCAGGAGAGTGTCACAGAGCAGGA |
| | | CAGCAAGGACAGCACCTACAGCCTCAGCAGCACCCTGACGCT |
| | | GAGCAAAGCAGACTACGAGAAACACAAAGTCTACGCCTGCGAA |
| | | GTCACCCATCAGGGCCTGAGCTCGCCCGTCACAAAGAGCTTCA |
| | | ACCGCGGAGAGTGT |
| NOV589HC | | |
| SEQ ID NO: 325 | HCDR1 | GFTFSKNGMH |
| (Combined) | | |
| SEQ ID NO: 326 | HCDR2 | MIYYDSSRMYYADTVKG |
| (Combined) | | |
| SEQ ID NO: 327 | HCDR3 | FVWVDLDFDY |
| (Combined) | | |
| SEQ ID NO: 328 | HCDR1 | KNGMH |
| (Kabat) | | |
| SEQ ID NO: 329 | HCDR2 | MIYYDSSRMYYADTVKG |
| (Kabat) | | |
| SEQ ID NO: 330 | HCDR3 | FWWDLDFDY |
| (Kabat) | | |
| SEQ ID NO: 331 | HCDR1 | GFTFSKN |
| (Chothia) | | |
| SEQ ID NO: 332 | HCDR2 | YYDSSR |
| (Chothia) | | |
| SEQ ID NO: 333 | HCDR3 | FWWDLDFDY |
| (Chothia) | | |
| SEQ ID NO: 334 | HCDR1 | GFTFSKNG |
| (IMGT) | | |
| SEQ ID NO: 335 | HCDR2 | IYYDSSRM |
| (IMGT) | | |
| SEQ ID NO: 336 | HCDR3 | ASFWWDLDFDY |
| (IMGT) | | |
| SEQ ID NO: 337 | VH | QVQLVESGGGVVQPGRSLRLSCAASGFTFSKNGMHWVRQAPGK |
| | | GLEWVAMIYYDSSRMYYADTVKGRFTISRDNSKNTLYLQMNSLRA |
| | | EDTAVYYCASFWWDLDFDYWGQGTMVTVSS |
| SEQ ID NO: 338 | DNA VH | AGGTGCAGCTGGTGGAGTCTGGAGGAGGAGTGGTGCAGCCAG |
| | | GCCGGTCCCTGAGACTGTCTTGCGCCGCCAGCGGCTTCACCTT |
| | | TAGCAAGAACGGAATGCACTGGGTGCGGCAGGCACCTGGCAA |
| | | GGGACTGGAGTGGGTGGCCATGATCTACTATGATAGCTCCAGG |
| | | ATGTACTATGCCGACACCGTGAAGGGCAGGTTCACAATCTCCC |
| | | GCGATAACTCTAAGAATACCCTGTACCTGCAGATGAATAGCCTG |
| | | CGGGCCGAGGACACAGCCGTGTACTATTGTGCCTCCTTCTGGT |
| | | GGGACCTGGATTTTGACTATTGGGGCCAGGGCACCATGGTGAC |
| | | AGTGTCTAGC |
| SEQ ID NO: 339 | Heavy | QVQLVESGGGVVQPGRSLRLSCAASGFTFSKNGMHWVRQAPG |
| | Chain | KGLEWVAMIYYDSSRMYYADTVKGRFTISRDNSKNTLYLQMNSLR |
| | | AEDTAVYYCASFWWDLDFDYWGQGTMVTVSSASTKGPSVFPLA |
| | | PSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKS |
| | | CDKTHTCPPCPAPEAAGGPSVFLFPPKPKDTLMISRTPEVTCVVV |
| | | DVSHEDPEVKFNWYVDGVEVHNAKTKPREEQYNSTYRVVSVLTV |
| | | LHQDWLNGKEYKCKVSNKALPAPIEKTISKAKGQPREPQVYTLPP |
| | | SREEMTKNQVSLTCLVKGFYPSDIAVEWESNGQPENNYKTTPPVL |
| | | DSDGSFFLYSKLTVDKSRWQQGNVFSCSVMHEALHNHYTQKSLS |
| | | LSPGK |
| SEQ ID NO: 340 | DNA | AGGTGCAGCTGGTGGAGTCTGGAGGAGGAGTGGTGCAGCCA |
| | Heavy | GGCCGGTCCCTGAGACTGTCTTGCGCCGCCAGCGGCTTCACC |
| | Chain | TTTAGCAAGAACGGAATGCACTGGGTGCGGCAGGCACCTGGC |
| | | AAGGGACTGGAGTGGGTGGCCATGATCTACTATGATAGCTCCA |
| | | GGATGTACTATGCCGACACCGTGAAGGGCAGGTTCACAATCTC |
| | | CCGCGATAACTCTAAGAATACCCTGTACCTGCAGATGAATAGC |
| | | CTGCGGGCCGAGGACACAGCCGTGTACTATTGTGCCTCCTTCT |
| | | GGTGGGACCTGGATTTTGACTATTGGGGCCAGGGCACCATGGT |
| | | GACAGTGTCTAGCGCTAGCACCAAGGGCCCATCGGTCTTCCCC |
| | | CTGGCACCCTCCTCCAAGAGCACCTCTGGGGGCACAGCGGCC |
| | | CTGGGCTGCCTGGTCAAGGACTACTTCCCCGAACCGGTGACG |
| | | GTGTCGTGGAACTCAGGCGCCCTGACCAGCGGCGTGCACACC |
| | | TTCCCGGCTGTCCTACAGTCCTCAGGACTCTACTCCCTCAGCA |
| | | GCGTGGTGACCGTGCCCTCCAGCAGCTTGGGCACCCAGACCT |
| | | ACATCTGCAACGTGAATCACAAGCCCAGCAACACCAAGGTGGA |
| | | CAAGAGAGTTGAGCCCAAATCTTGTGACAAAACTCACACATGC |
| | | CCACCGTGCCCAGCACCTGAAGCCGCTGGGGGACCGTCAGTC |
| | | TTCCTCTTCCCCCCAAAACCCAAGGACACCCTCATGATCTCCC |
| | | GGACCCCTGAGGTCACATGCGTGGTGGTGGACGTGAGCCACG |
| | | AAGACCCTGAGGTCAAGTTCAACTGGTACGTGGACGGCGTGGA |
| | | GGTGCATAATGCCAAGACAAAGCCGCGGGAGGAGCAGTACAA |
| | | CAGCACGTACCGTGTGGTCAGCGTCCTCACCGTCCTGCACCAG |
| | | GACTGGCTGAATGGCAAGGAGTACAAGTGCAAGGTCTCCAACA |
| | | AAGCCCTCCCAGCCCCCATCGAGAAAACCATCTCCAAAGCCAA |
| | | AGGGCAGCCCCGAGAACCACAGGTGTACACCCTGCCCCCATC |
| | | CCGGGAGGAGATGACCAAGAACCAGGTCAGCCTGACCTGCCT |
| | | GGTCAAAGGCTTCTATCCCAGCGACATCGCCGTGGAGTGGGA |
| | | GAGCAATGGGCAGCCGGAGAACAACTACAAGACCACGCCTCC |
| | | CGTGCTGGACTCCGACGGCTCCTTCTTCCTCTACAGCAAGCTC |
| | | ACCGTGGACAAGAGCAGGTGGCAGCAGGGGAACGTCTTCTCA |
| | | TGCTCCGTGATGCATGAGGCTCTGCACAACCACTACACGCAGA |
| | | AGAGCCTCTCCCTGTCTCCGGGTAAA |
| NOV589 LC | | |
| SEQ ID NO: 341 | LCDR1 | RSSQSLVRSDGTTYFN |
| (Combined) | | |
| SEQ ID NO: 342 | LCDR2 | RVSNRFS |
| (Combined) | | |
| SEQ ID NO: 343 | LCDR3 | LQSSHFPVVT |
| (Combined) | | |
| SEQ ID NO: 344 | LCDR1 | RSSQSLVRSDGTTYFN |
| (Kabat) | | |
| SEQ ID NO: 345 | LCDR2 | RVSNRFS |
| (Kabat) | | |
| SEQ ID NO: 346 | LCDR3 | LQSSHFPWT |
| (Kabat) | | |
| SEQ ID NO: 347 | LCDR1 | SQSLVRSDGTTY |
| (Chothia) | | |
| SEQ ID NO: 348 | LCDR2 | RVS |
| (Chothia) | | |
| SEQ ID NO: 349 | LCDR3 | SSHFPW |
| (Chothia) | | |
| SEQ ID NO: 350 | LCDR1 | QSLVRSDGTTY |
| (IMGT) | | |
| SEQ ID NO: 351 | LCDR2 | RVS |
| (IMGT) | | |
| SEQ ID NO: 352 | LCDR3 | LQSSHFPWT |
| (IMGT) | | |
| SEQ ID NO: 353 | VL | DIVMTQTPLSSPVTLGQPASISCRSSQSLVRSDGTTYFNWYQQRP |
| | | GQPPRLLIYRVSNRFSGVPDRFSGSGAGTDFTLKISRVEAEDVGV |
| | | YYCLQSSHFPWTFGGGTKVEIK |
| SEQ ID NO: 354 | DNA VL | GACATCGTGATGACCCAGACACCACTGAGCTCCCCAGTGACCC |
| | | TGGGACAGCCAGCCAGCATCTCCTGCCGGTCTAGCCAGTCCCT |
| | | GGTGAGATCTGATGGCACCACATACTTCAACTGGTATCAGCAG |
| | | AGGCCTGGACAGCCACCTAGGCTGCTGATCTACCGGGTGAGC |
| | | AATAGATTCTCCGGCGTGCCAGACAGGTTTTCTGGCAGCGGAG |
| | | CAGGAACCGACTTCACCCTGAAGATCTCTAGAGTGGAGGCCGA |
| | | GGACGTGGGCGTGTACTATTGTCTGCAGTCCTCTCACTTCCCT |
| | | TGGACCTTTGGCGGCGGCACAAAGGTGGAGATCAAG |
| SEQ ID NO: 355 | Light | DIVMTQTPLSSPVTLGQPASISCRSSQSLVRSDGTTYFNWYQQRP |
| | chain | GQPPRLLIYRVSNRFSGVPDRFSGSGAGTDFTLKISRVEAEDVGV |
| | | YYCLQSSHFPWTFGGGTKVEIKRTVAAPSVFIFPPSDEQLKSGTAS |
| | | VVCLLNNFYPREAKVQWKVDNALQSGNSQESVTEQDSKDSTYSL |
| | | SSTLTLSKADYEKHKVYACEVTHQGLSSPVTKSFNRGEC |
| SEQ ID NO: 356 | DNA | GACATCGTGATGACCCAGACACCACTGAGCTCCCCAGTGACCC |
| | Light | TGGGACAGCCAGCCAGCATCTCCTGCCGGTCTAGCCAGTCCCT |
| | Chain | GGTGAGATCTGATGGCACCACATACTTCAACTGGTATCAGCAG |
| | | AGGCCTGGACAGCCACCTAGGCTGCTGATCTACCGGGTGAGC |
| | | AATAGATTCTCCGGCGTGCCAGACAGGTTTTCTGGCAGCGGAG |
| | | CAGGAACCGACTTCACCCTGAAGATCTCTAGAGTGGAGGCCGA |
| | | GGACGTGGGCGTGTACTATTGTCTGCAGTCCTCTCACTTCCCT |
| | | TGGACCTTTGGCGGCGGCACAAAGGTGGAGATCAAGCGTACG |
| | | GTGGCTGCACCATCTGTCTTCATCTTCCCGCCATCTGATGAGC |
| | | AGTTGAAATCTGGAACTGCCTCTGTTGTGTGCCTGCTGAATAAC |
| | | TTCTATCCCCGCGAGGCCAAAGTACAGTGGAAGGTGGATAACG |
| | | CCCTCCAATCGGGTAACTCCCAGGAGAGTGTCACAGAGCAGGA |
| | | CAGCAAGGACAGCACCTACAGCCTCAGCAGCACCCTGACGCT |
| | | GAGCAAAGCAGACTACGAGAAACACAAAGTCTACGCCTGCGAA |
| | | GTCACCCATCAGGGCCTGAGCTCGCCCGTCACAAAGAGCTTCA |
| | | ACCGCGGAGAGTGT |
| NOV580 HC | | |
| SEQ ID NO: 357 | HCDR1 | GFSLTTYNIH |
| (Combined) | | |
| SEQ ID NO: 358 | HCDR2 | RMRYSGDTSYSSALKS |
| (Combined) | | |
| SEQ ID NO: 359 | HCDR3 | DPMYIPGYSYGVMNA |
| (Combined) | | |
| SEQ ID NO: 360 | HCDR1 | TYNIH |
| (Kabat) | | |
| SEQ ID NO: 361 | HCDR2 | RMRYSGDTSYSSALKS |
| (Kabat) | | |
| SEQ ID NO: 362 | HCDR3 | DPMYIPGYSYGVMNA |
| (Kabat) | | |
| SEQ ID NO: 363 | HCDR1 | GFSLTTY |
| (Chothia) | | |
| SEQ ID NO: 364 | HCDR2 | RYSGD |
| (Chothia) | | |
| SEQ ID NO: 365 | HCDR3 | DPMYIPGYSYGVMNA |
| (Chothia) | | |
| SEQ ID NO: 366 | HCDR1 | GFSLTTYN |
| (IMGT) | | |
| SEQ ID NO: 367 | HCDR2 | MRYSGDT |
| (IMGT) | | |
| SEQ ID NO: 368 | HCDR3 | TRDPMYIPGYSYGVMNA |
| (IMGT) | | |
| SEQ ID NO: 369 | VH | QVQLQESGPGLVKPSETLSLTCTVSGFSLTTYNIHWIRQPPGKGLE |
| | | WIGRMRYSGDTSYSSALKSRVTISRDTSKNQVSLKLSSVTAADTA |
| | | VYYCTRDPMYIPGYSYGVMNAWGQGTTVTVSS |
| SEQ ID NO: 370 | DNA VH | CAGGTGCAGCTGCAGGAGTCCGGACCTGGACTGGTGAAGCCA |
| | | TCTGAGACCCTGAGCCTGACCTGCACAGTGAGCGGCTTCTCCC |
| | | TGACCACATACAACATCCACTGGATCAGACAGCCACCTGGCAA |
| | | GGGACTGGAGTGGATCGGCCGGATGAGATACTCCGGCGACAC |
| | | ATCTTATAGCTCCGCCCTGAAGTCTAGGGTGACCATCAGCCGC |
| | | GATACATCCAAGAACCAGGTGAGCCTGAAGCTGTCTAGCGTGA |
| | | CCGCCGCCGACACAGCCGTGTACTATTGTACCCGGGACCCCAT |
| | | GTATATCCCCGGCTACTCTTATGGCGTGATGAATGCCTGGGGC |
| | | CAGGGCACCACAGTGACAGTGTCCTCT |
| SEQ ID NO: 371 | Heavy | QVQLQESGPGLVKPSETLSLTCTVSGFSLTTYNIHWIRQPPGKGL |
| | Chain | EWIGRMRYSGDTSYSSALKSRVTISRDTSKNQVSLKLSSVTAADT |
| | | AVYYCTRDPMYIPGYSYGVMNAWGQGTTVTVSSASTKGPSVFPL |
| | | APSSKSTSGGTAALGCLVKDYFPEPVTVSWNSGALTSGVHTFPAV |
| | | LQSSGLYSLSSVVTVPSSSLGTQTYICNVNHKPSNTKVDKRVEPK |
| | | SCDKTHTCPPCPAPEAAGGPSVFLFPPKPKDTLMISRTPEVTCVV |
| | | VDVSHEDPEVKFNWYVDGVEVHNAKTKPREEQYNSTYRVVSVLT |
| | | VLHQDWLNGKEYKCKVSNKALPAPIEKTISKAKGQPREPQVYTLP |
| | | PSREEMTKNQVSLTCLVKGFYPSDIAVEWESNGQPENNYKTTPPV |
| | | LDSDGSFFLYSKLTVDKSRWQQGNVFSCSVMHEALHNHYTQKSL |
| | | SLSPGK |
| SEQ ID NO: 372 | DNA | CAGGTGCAGCTGCAGGAGTCCGGACCTGGACTGGTGAAGCCA |
| | Heavy | TCTGAGACCCTGAGCCTGACCTGCACAGTGAGCGGCTTCTCCC |
| | Chain | TGACCACATACAACATCCACTGGATCAGACAGCCACCTGGCAA |
| | | GGGACTGGAGTGGATCGGCCGGATGAGATACTCCGGCGACAC |
| | | ATCTTATAGCTCCGCCCTGAAGTCTAGGGTGACCATCAGCCGC |
| | | GATACATCCAAGAACCAGGTGAGCCTGAAGCTGTCTAGCGTGA |
| | | CCGCCGCCGACACAGCCGTGTACTATTGTACCCGGGACCCCAT |
| | | GTATATCCCCGGCTACTCTTATGGCGTGATGAATGCCTGGGGC |
| | | CAGGGCACCACAGTGACAGTGTCCTCTGCTAGCACCAAGGGC |
| | | CCATCGGTCTTCCCCCTGGCACCCTCCTCCAAGAGCACCTCTG |
| | | GGGGCACAGCGGCCCTGGGCTGCCTGGTCAAGGACTACTTCC |
| | | CCGAACCGGTGACGGTGTCGTGGAACTCAGGCGCCCTGACCA |
| | | GCGGCGTGCACACCTTCCCGGCTGTCCTACAGTCCTCAGGACT |
| | | CTACTCCCTCAGCAGCGTGGTGACCGTGCCCTCCAGCAGCTTG |
| | | GGCACCCAGACCTACATCTGCAACGTGAATCACAAGCCCAGCA |
| | | ACACCAAGGTGGACAAGAGAGTTGAGCCCAAATCTTGTGACAA |
| | | AACTCACACATGCCCACCGTGCCCAGCACCTGAAGCCGCTGG |
| | | GGGACCGTCAGTCTTCCTCTTCCCCCCAAAACCCAAGGACACC |
| | | CTCATGATCTCCCGGACCCCTGAGGTCACATGCGTGGTGGTGG |
| | | ACGTGAGCCACGAAGACCCTGAGGTCAAGTTCAACTGGTACGT |
| | | GGACGGCGTGGAGGTGCATAATGCCAAGACAAAGCCGCGGGA |
| | | GGAGCAGTACAACAGCACGTACCGTGTGGTCAGCGTCCTCACC |
| | | GTCCTGCACCAGGACTGGCTGAATGGCAAGGAGTACAAGTGCA |
| | | AGGTCTCCAACAAAGCCCTCCCAGCCCCCATCGAGAAAACCAT |
| | | CTCCAAAGCCAAAGGGCAGCCCCGAGAACCACAGGTGTACAC |
| | | CCTGCCCCCATCCCGGGAGGAGATGACCAAGAACCAGGTCAG |
| | | CCTGACCTGCCTGGTCAAAGGCTTCTATCCCAGCGACATCGCC |
| | | GTGGAGTGGGAGAGCAATGGGCAGCCGGAGAACAACTACAAG |
| | | ACCACGCCTCCCGTGCTGGACTCCGACGGCTCCTTCTTCCTCT |
| | | ACAGCAAGCTCACCGTGGACAAGAGCAGGTGGCAGCAGGGGA |
| | | ACGTCTTCTCATGCTCCGTGATGCATGAGGCTCTGCACAACCA |
| | | CTACACGCAGAAGAGCCTCTCCCTGTCTCCGGGTAAA |
| NOV580 LC | | |
| SEQ ID NO: 373 | LCDR1 | KTSQNIDKYLN |
| (Combined) | | |
| SEQ ID NO: 374 | LCDR2 | NTNNLEA |
| (Combined) | | |
| SEQ ID NO: 375 | LCDR3 | LQHRSSYT |
| (Combined) | | |
| SEQ ID NO: 376 | LCDR1 | KTSQNIDKYLN |
| (Kabat) | | |
| SEQ ID NO: 377 | LCDR2 | NTNNLEA |
| (Kabat) | | |
| SEQ ID NO: 378 | LCDR3 | LQHRSSYT |
| (Kabat) | | |
| SEQ ID NO: 379 | LCDR1 | SQNIDKY |
| (Chothia) | | |
| SEQ ID NO: 380 | LCDR2 | NTN |
| (Chothia) | | |
| SEQ ID NO: 381 | LCDR3 | HRSSY |
| (Chothia) | | |
| SEQ ID NO: 382 | LCDR1 | QNIDKY |
| (IMGT) | | |
| SEQ ID NO: 383 | LCDR2 | NTNNLEAGVP |
| (IMGT) | | |
| SEQ ID NO: 384 | LCDR3 | LQHRSSYT |
| (IMGT) | | |
| SEQ ID NO: 385 | VL | DIQMTQSPSSLSASVGDRVTITCKTSQNIDKYLNWYQQKPGKAPK |
| | | LLIYNTNNLEAGVPSRFSGSGSGTDYTFTISSLQPEDIATYFCLQHR |
| | | SSYTFGQGTKLEIK |
| SEQ ID NO: 386 | DNA VL | GACATCCAGATGACCCAGTCTCCAAGCTCCCTGAGCGCCTCCG |
| | | TGGGCGACAGGGTGACCATCACATGCAAGACAAGCCAGAACAT |
| | | CGATAAGTACCTGAATTGGTATCAGCAGAAGCCCGGCAAGGCC |
| | | CCTAAGCTGCTGATCTACAACACCAACAATCTGGAGGCAGGAG |
| | | TGCCATCCCGGTTCTCTGGCAGCGGCTCCGGAACCGACTATAC |
| | | CTTTACAATCTCTAGCCTGCAGCCCGAGGATATCGCCACATACT |
| | | TCTGTCTGCAGCACAGATCCTCTTATACCTTTGGCCAGGGCAC |
| | | AAAGCTGGAGATCAAG |
| SEQ ID NO: 387 | Light | DIQMTQSPSSLSASVGDRVTITCKTSQNIDKYLNWYQQKPGKAPK |
| | chain | LLIYNTNNLEAGVPSRFSGSGSGTDYTFTISSLQPEDIATYFCLQHR |
| | | SSYTFGQGTKLEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC |
| SEQ ID NO: 388 | DNA | GACATCCAGATGACCCAGTCTCCAAGCTCCCTGAGCGCCTCCG |
| | Light | TGGGCGACAGGGTGACCATCACATGCAAGACAAGCCAGAACAT |
| | Chain | CGATAAGTACCTGAATTGGTATCAGCAGAAGCCCGGCAAGGCC |
| | | CCTAAGCTGCTGATCTACAACACCAACAATCTGGAGGCAGGAG |
| | | TGCCATCCCGGTTCTCTGGCAGCGGCTCCGGAACCGACTATAC |
| | | CTTTACAATCTCTAGCCTGCAGCCCGAGGATATCGCCACATACT |
| | | TCTGTCTGCAGCACAGATCCTCTTATACCTTTGGCCAGGGCAC |
| | | AAAGCTGGAGATCAAGCGTACGGTGGCTGCACCATCTGTCTTC |
| | | ATCTTCCCGCCATCTGATGAGCAGTTGAAATCTGGAACTGCCTC |
| | | TGTTGTGTGCCTGCTGAATAACTTCTATCCCCGCGAGGCCAAA |
| | | GTACAGTGGAAGGTGGATAACGCCCTCCAATCGGGTAACTCCC |
| | | AGGAGAGTGTCACAGAGCAGGACAGCAAGGACAGCACCTACA |
| | | GCCTCAGCAGCACCCTGACGCTGAGCAAAGCAGACTACGAGA |
| | | AACACAAAGTCTACGCCTGCGAAGTCACCCATCAGGGCCTGAG |
| | | CTCGCCCGTCACAAAGAGCTTCAACCGCGGAGAGTGT |
| NOV567 HC | | |
| SEQ ID NO: 389 | HCDR1 | GFAFRKYGMS |
| (Combined) | | |
| SEQ ID NO: 390 | HCDR2 | LIYYDSSKMNYADTVKG |
| (Combined) | | |
| SEQ ID NO: 391 | HCDR3 | LNSEYD |
| (Combined) | | |
| SEQ ID NO: 392 | HCDR1 | KYGMS |
| (Kabat) | | |
| SEQ ID NO: 393 | HCDR2 | LIYYDSSKMNYADTVKG |
| (Kabat) | | |
| SEQ ID NO: 394 | HCDR3 | LNSEYD |
| (Kabat) | | |
| SEQ ID NO: 395 | HCDR1 | GFAFRKY |
| (Chothia) | | |
| SEQ ID NO: 396 | HCDR2 | YYDSSK |
| (Chothia) | | |
| SEQ ID NO: 397 | HCDR3 | LNSEYD |
| (Chothia) | | |
| SEQ ID NO: 398 | HCDR1 | GFAFRKYG |
| (IMGT) | | |
| SEQ ID NO: 399 | HCDR2 | IYYDSSKM |
| (IMGT) | | |
| SEQ ID NO: 400 | HCDR3 | AALNSEYD |
| (IMGT) | | |
| SEQ ID NO: 401 | VH | QVQLVESGGGVVQPGRSLRLSCAASGFAFRKYGMSWVRQAPGK |
| | | GLEWVALIYYDSSKMNYADTVKGRFTISRDNSKNTLYLQMNSLRA |
| | | EDTAVYYCAALNSEYDWGQGTMVTVSS |
| SEQ ID NO: 402 | DNA VH | CAGGTGCAGCTGGTGGAGTCTGGAGGAGGAGTGGTGCAGCCA |
| | | GGCCGGTCCCTGAGACTGTCTTGCGCCGCCAGCGGCTTCGCC |
| | | TTTCGGAAGTACGGAATGAGCTGGGTGAGGCAGGCACCTGGC |
| | | AAGGGACTGGAGTGGGTGGCCCTGATCTACTATGACAGCTCCA |
| | | AGATGAACTACGCCGATACCGTGAAGGGCAGGTTCACAATCTC |
| | | CCGCGACAACTCTAAGAATACCCTGTATCTGCAGATGAACAGC |
| | | CTGCGGGCCGAGGACACAGCCGTGTACTATTGTGCCGCCCTG |
| | | AATTCCGAGTATGATTGGGGCCAGGGCACCATGGTGACAGTGT |
| | | CTAGC |
| SEQ ID NO: 403 | Heavy | QVQLVESGGGVVQPGRSLRLSCAASGFAFRKYGMSWVRQAPG |
| | Chain | KGLEWVALIYYDSSKMNYADTVKGRFTISRDNSKNTLYLQMNSLR |
| | | AEDTAVYYCAALNSEYDWGQGTMVTVSSASTKGPSVFPLAPSSK |
| | | STSGGTAALGCLVKDYFPEPVTVSWNSGALTSGVHTFPAVLQSS |
| | | GLYSLSSVVTVPSSSLGTQTYICNVNHKPSNTKVDKRVEPKSCDK |
| | | THTCPPCPAPEAAGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKALPAPIEKTISKAKGQPREPQVYTLPPSRE |
| | | EMTKNQVSLTCLVKGFYPSDIAVEWESNGQPENNYKTTPPVLDSD |
| | | GSFFLYSKLTVDKSRWQQGNVFSCSVMHEALHNHYTQKSLSLSP |
| | | GK |
| SEQ ID NO: 404 | DNA | CAGGTGCAGCTGGTGGAGTCTGGAGGAGGAGTGGTGCAGCC |
| | Heavy | AGGCCGGTCCCTGAGACTGTCTTGCGCCGCCAGCGGCTTCGC |
| | Chain | CTTTCGGAAGTACGGAATGAGCTGGGTGAGGCAGGCACCTGG |
| | | CAAGGGACTGGAGTGGGTGGCCCTGATCTACTATGACAGCTCC |
| | | AAGATGAACTACGCCGATACCGTGAAGGGCAGGTTCACAATCT |
| | | CCCGCGACAACTCTAAGAATACCCTGTATCTGCAGATGAACAG |
| | | CCTGCGGGCCGAGGACACAGCCGTGTACTATTGTGCCGCCCT |
| | | GAATTCCGAGTATGATTGGGGCCAGGGCACCATGGTGACAGTG |
| | | TCTAGCGCTAGCACCAAGGGCCCATCGGTCTTCCCCCTGGCAC |
| | | CCTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCGG |
| | | CTGTCCTACAGTCCTCAGGACTCTACTCCCTCAGCAGCGTGGT |
| | | GACCGTGCCCTCCAGCAGCTTGGGCACCCAGACCTACATCTGC |
| | | AACGTGAATCACAAGCCCAGCAACACCAAGGTGGACAAGAGAG |
| | | TTGAGCCCAAATCTTGTGACAAAACTCACACATGCCCACCGTG |
| | | CCCAGCACCTGAAGCCGCTGGGGGACCGTCAGTCTTCCTCTTC |
| | | CCCCCAAAACCCAAGGACACCCTCATGATCTCCCGGACCCCTG |
| | | AGGTCACATGCGTGGTGGTGGACGTGAGCCACGAAGACCCTG |
| | | AGGTCAAGTTCAACTGGTACGTGGACGGCGTGGAGGTGCATAA |
| | | TGCCAAGACAAAGCCGCGGGAGGAGCAGTACAACAGCACGTA |
| | | CCGTGTGGTCAGCGTCCTCACCGTCCTGCACCAGGACTGGCT |
| | | GAATGGCAAGGAGTACAAGTGCAAGGTCTCCAACAAAGCCCTC |
| | | CCAGCCCCCATCGAGAAAACCATCTCCAAAGCCAAAGGGCAGC |
| | | CCCGAGAACCACAGGTGTACACCCTGCCCCCATCCCGGGAGG |
| | | AGATGACCAAGAACCAGGTCAGCCTGACCTGCCTGGTCAAAGG |
| | | CTTCTATCCCAGCGACATCGCCGTGGAGTGGGAGAGCAATGG |
| | | GCAGCCGGAGAACAACTACAAGACCACGCCTCCCGTGCTGGA |
| | | CTCCGACGGCTCCTTCTTCCTCTACAGCAAGCTCACCGTGGAC |
| | | AAGAGCAGGTGGCAGCAGGGGAACGTCTTCTCATGCTCCGTG |
| | | ATGCATGAGGCTCTGCACAACCACTACACGCAGAAGAGCCTCT |
| | | CCCTGTCTCCGGGTAAA |
| NOV567 LC | | |
| SEQ ID NO: 405 | LCDR1 | RGSQSIGNSLN |
| (Combined) | | |
| SEQ ID NO: 406 | LCDR2 | STSTLEY |
| (Combined) | | |
| SEQ ID NO: 407 | LCDR3 | LQYATYPYT |
| (Combined) | | |
| SEQ ID NO: 408 | LCDR1 | RGSQSIGNSLN |
| (Kabat) | | |
| SEQ ID NO: 409 | LCDR2 | STSTLEY |
| (Kabat) | | |
| SEQ ID NO: 410 | LCDR3 | LQYATYPYT |
| (Kabat) | | |
| SEQ ID NO: 411 | LCDR1 | SQSIGNS |
| (Chothia) | | |
| SEQ ID NO: 412 | LCDR2 | STS |
| (Chothia) | | |
| SEQ ID NO: 413 | LCDR3 | YATYPY |
| (Chothia) | | |
| SEQ ID NO: 414 | LCDR1 | QSIGNS |
| (IMGT) | | |
| SEQ ID NO: 415 | LCDR2 | STSTLEYGVP |
| (IMGT) | | |
| SEQ ID NO: 416 | LCDR3 | LQYATYPYT |
| (IMGT) | | |
| SEQ ID NO: 417 | VL | DIQMTQSPSSLSASVGDRVTITCRGSQSIGNSLNWYQQKPGKAPK |
| | | RLIYSTSTLEYGVPSRFSGSGSGTEYTLTISSLQPEDFATYYCLQY |
| | | ATYPYTFGQGTKLEIK |
| SEQ ID NO: 418 | DNA VL | GACATCCAGATGACCCAGTCCCCTAGCTCCCTGTCCGCCTCTG |
| | | TGGGCGATAGGGTGACCATCACATGCAGAGGCAGCCAGTCCA |
| | | TCGGCAACTCTCTGAATTGGTACCAGCAGAAGCCCGGCAAGGC |
| | | CCCTAAGAGGCTGATCTACTCTACCAGCACACTGGAGTATGGA |
| | | GTGCCATCCCGGTTCTCCGGCTCTGGCAGCGGAACCGAGTAC |
| | | ACCCTGACAATCTCTAGCCTGCAGCCAGAGGACTTCGCCACAT |
| | | ACTATTGTCTGCAGTATGCCACCTACCCCTATACATTTGGCCAG |
| | | GGCACAAAGCTGGAGATCAAG |
| SEQ ID NO: 419 | Light | DIQMTQSPSSLSASVGDRVTITCRGSQSIGNSLNWYQQKPGKAPK |
| | chain | RLIYSTSTLEYGVPSRFSGSGSGTEYTLTISSLQPEDFATYYCLQY |
| | | ATYPYTFGQGTKLEIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| SEQ ID NO: 420 | DNA | GACATCCAGATGACCCAGTCCCCTAGCTCCCTGTCCGCCTCTG |
| | Light | TGGGCGATAGGGTGACCATCACATGCAGAGGCAGCCAGTCCA |
| | Chain | TCGGCAACTCTCTGAATTGGTACCAGCAGAAGCCCGGCAAGGC |
| | | CCCTAAGAGGCTGATCTACTCTACCAGCACACTGGAGTATGGA |
| | | GTGCCATCCCGGTTCTCCGGCTCTGGCAGCGGAACCGAGTAC |
| | | ACCCTGACAATCTCTAGCCTGCAGCCAGAGGACTTCGCCACAT |
| | | ACTATTGTCTGCAGTATGCCACCTACCCCTATACATTTGGCCAG |
| | | GGCACAAAGCTGGAGATCAAGCGTACGGTGGCTGCACCATCT |
| | | GTCTTCATCTTCCCGCCATCTGATGAGCAGTTGAAATCTGGAAC |
| | | TGCCTCTGTTGTGTGCCTGCTGAATAACTTCTATCCCCGCGAG |
| | | GCCAAAGTACAGTGGAAGGTGGATAACGCCCTCCAATCGGGTA |
| | | ACTCCCAGGAGAGTGTCACAGAGCAGGACAGCAAGGACAGCA |
| | | CCTACAGCCTCAGCAGCACCCTGACGCTGAGCAAAGCAGACTA |
| | | CGAGAAACACAAAGTCTACGCCTGCGAAGTCACCCATCAGGGC |
| | | CTGAGCTCGCCCGTCACAAAGAGCTTCAACCGCGGAGAGTGT |
| NOV221 HC | | |
| SEQ ID NO: 421 | HCDR1 | GFSLTTYNIH |
| (Combined) | | |
| SEQ ID NO: 422 | HCDR2 | RMRYSGDTSYSSALKS |
| (Combined) | | |
| SEQ ID NO: 423 | HCDR3 | DPMYIPGYSYGVMNA |
| (Combined) | | |
| SEQ ID NO: 424 | HCDR1 | TYNIH |
| (Kabat) | | |
| SEQ ID NO: 425 | HCDR2 | RMRYSGDTSYSSALKS |
| (Kabat) | | |
| SEQ ID NO: 426 | HCDR3 | DPMYIPGYSYGVMNA |
| (Kabat) | | |
| SEQ ID NO: 427 | HCDR1 | GFSLTTY |
| (Chothia) | | |
| SEQ ID NO: 428 | HCDR2 | RYSGD |
| (Chothia) | | |
| SEQ ID NO: 429 | HCDR3 | DPMYIPGYSYGVMNA |
| (Chothia) | | |
| SEQ ID NO: 430 | HCDR1 | GFSLTTYN |
| (IMGT) | | |
| SEQ ID NO: 431 | HCDR2 | MRYSGDT |
| (IMGT) | | |
| SEQ ID NO: 432 | HCDR3 | TRDPMYIPGYSYGVMNA |
| (IMGT) | | |
| SEQ ID NO: 433 | VH | QVQLQESGPGLVKPSETLSLTCTVSGFSLTTYNIHWIRQPPGKGLE |
| | | WIGRMRYSGDTSYSSALKSRVTISRDTSKNQVSLKLSSVTAADTA |
| | | VYYCTRDPMYIPGYSYGVMNAWGQGTTVTVSS |
| SEQ ID NO: 434 | DNA VH | CAGGTGCAGCTGCAGGAGTCCGGACCTGGACTGGTGAAGCCA |
| | | TCTGAGACCCTGAGCCTGACCTGCACAGTGAGCGGCTTCTCCC |
| | | TGACCACATACAACATCCACTGGATCAGACAGCCACCTGGCAA |
| | | GGGACTGGAGTGGATCGGCCGGATGAGATACTCCGGCGACAC |
| | | ATCTTATAGCTCCGCCCTGAAGTCTAGGGTGACCATCAGCCGC |
| | | GATACATCCAAGAACCAGGTGAGCCTGAAGCTGTCTAGCGTGA |
| | | CCGCCGCCGACACAGCCGTGTACTATTGTACCCGGGACCCCAT |
| | | GTATATCCCCGGCTACTCTTATGGCGTGATGAATGCCTGGGGC |
| | | CAGGGCACCACAGTGACAGTGTCCTCT |
| SEQ ID NO: 435 | Heavy | QVQLQESGPGLVKPSETLSLTCTVSGFSLTTYNIHWIRQPPGKGL |
| | Chain | EWIGRMRYSGDTSYSSALKSRVTISRDTSKNQVSLKLSSVTAADT |
| | | AVYYCTRDPMYIPGYSYGVMNAWGQGTTVTVSSASTKGPSVFPL |
| | | APSSKSTSGGTAALGCLVKDYFPEPVTVSWNSGALTSGVHTFPAV |
| | | LQSSGLYSLSSVVTVPSSSLGTQTYICNVNHKPSNTKVDKRVEPK |
| | | SCDKTHTCPPCPAPEAAGGPSVFLFPPKPKDTLMISRTPEVTCVV |
| | | VDVSHEDPEVKFNWYVDGVEVHNAKTKPREEQYNSTYRVVSVLT |
| | | VLHQDWLNGKEYKCKVSNKALPAPIEKTISKAKGQPREPQVYTLP |
| | | PSREEMTKNQVSLTCLVKGFYPSDIAVEWESNGQPENNYKTTPPV |
| | | LDSDGSFFLYSKLTVDKSRWQQGNVFSCSVMHEALHNHYTQKSL |
| | | SLSPGK |
| SEQ ID NO: 436 | DNA | CAGGTGCAGCTGCAGGAGTCCGGACCTGGACTGGTGAAGCCA |
| | Heavy | TCTGAGACCCTGAGCCTGACCTGCACAGTGAGCGGCTTCTCCC |
| | Chain | TGACCACATACAACATCCACTGGATCAGACAGCCACCTGGCAA |
| | | GGGACTGGAGTGGATCGGCCGGATGAGATACTCCGGCGACAC |
| | | ATCTTATAGCTCCGCCCTGAAGTCTAGGGTGACCATCAGCCGC |
| | | GATACATCCAAGAACCAGGTGAGCCTGAAGCTGTCTAGCGTGA |
| | | CCGCCGCCGACACAGCCGTGTACTATTGTACCCGGGACCCCAT |
| | | GTATATCCCCGGCTACTCTTATGGCGTGATGAATGCCTGGGGC |
| | | CAGGGCACCACAGTGACAGTGTCCTCTGCTAGCACCAAGGGC |
| | | CCATCGGTCTTCCCCCTGGCACCCTCCTCCAAGAGCACCTCTG |
| | | GGGGCACAGCGGCCCTGGGCTGCCTGGTCAAGGACTACTTCC |
| | | CCGAACCGGTGACGGTGTCGTGGAACTCAGGCGCCCTGACCA |
| | | GCGGCGTGCACACCTTCCCGGCTGTCCTACAGTCCTCAGGACT |
| | | CTACTCCCTCAGCAGCGTGGTGACCGTGCCCTCCAGCAGCTTG |
| | | GGCACCCAGACCTACATCTGCAACGTGAATCACAAGCCCAGCA |
| | | ACACCAAGGTGGACAAGAGAGTTGAGCCCAAATCTTGTGACAA |
| | | AACTCACACATGCCCACCGTGCCCAGCACCTGAAGCCGCTGG |
| | | GGGACCGTCAGTCTTCCTCTTCCCCCCAAAACCCAAGGACACC |
| | | CTCATGATCTCCCGGACCCCTGAGGTCACATGCGTGGTGGTGG |
| | | ACGTGAGCCACGAAGACCCTGAGGTCAAGTTCAACTGGTACGT |
| | | GGACGGCGTGGAGGTGCATAATGCCAAGACAAAGCCGCGGGA |
| | | GGAGCAGTACAACAGCACGTACCGTGTGGTCAGCGTCCTCACC |
| | | GTCCTGCACCAGGACTGGCTGAATGGCAAGGAGTACAAGTGCA |
| | | AGGTCTCCAACAAAGCCCTCCCAGCCCCCATCGAGAAAACCAT |
| | | CTCCAAAGCCAAAGGGCAGCCCCGAGAACCACAGGTGTACAC |
| | | CCTGCCCCCATCCCGGGAGGAGATGACCAAGAACCAGGTCAG |
| | | CCTGACCTGCCTGGTCAAAGGCTTCTATCCCAGCGACATCGCC |
| | | GTGGAGTGGGAGAGCAATGGGCAGCCGGAGAACAACTACAAG |
| | | ACCACGCCTCCCGTGCTGGACTCCGACGGCTCCTTCTTCCTCT |
| | | ACAGCAAGCTCACCGTGGACAAGAGCAGGTGGCAGCAGGGGA |
| | | ACGTCTTCTCATGCTCCGTGATGCATGAGGCTCTGCACAACCA |
| | | CTACACGCAGAAGAGCCTCTCCCTGTCTCCGGGTAAA |
| NOV221 LC | | |
| SEQ ID NO: 437 | LCDR1 | KSSQNIDKYLN |
| (Combined) | | |
| SEQ ID NO: 438 | LCDR2 | NTNNLEA |
| (Combined) | | |
| SEQ ID NO: 439 | LCDR3 | LQHRSGYT |
| (Combined) | | |
| SEQ ID NO: 440 | LCDR1 | KSSQNIDKYLN |
| (Kabat) | | |
| SEQ ID NO: 441 | LCDR2 | NTNNLEA |
| (Kabat) | | |
| SEQ ID NO: 442 | LCDR3 | LQHRSGYT |
| (Kabat) | | |
| SEQ ID NO: 443 | LCDR1 | SQNIDKY |
| (Chothia) | | |
| SEQ ID NO: 444 | LCDR2 | NTN |
| (Chothia) | | |
| SEQ ID NO: 445 | LCDR3 | HRSGY |
| (Chothia) | | |
| SEQ ID NO: 446 | LCDR1 | QNIDKY |
| (IMGT) | | |
| SEQ ID NO: 447 | LCDR2 | NTNNLEAGVP |
| (IMGT) | | |
| SEQ ID NO: 448 | LCDR3 | LQHRSGYT |
| (IMGT) | | |
| SEQ ID NO: 449 | VL | DIQMTQSPSSLSASVGDRVTITCKSSQNIDKYLNWYQQKPGKAPK |
| | | LLIYNTNNLEAGVPSRFSGSGSGTDYTFTISSLQPEDIATYFCLQHR |
| | | SGYTFGQGTKLEIK |
| SEQ ID NO: 450 | DNA VL | GGATCCACCGGCGACATCCAGATGACCCAGTCCCCAAGCTCC |
| | | CTGAGCGCCTCCGTGGGCGACCGGGTGACCATCACATGCAAG |
| | | TCTAGCCAGAACATCGATAAGTACCTGAATTGGTATCAGCAGAA |
| | | GCCCGGCAAGGCCCCTAAGCTGCTGATCTACAACACAAACAAT |
| | | CTGGAGGCCGGCGTGCCATCTCGGTTCTCTGGCAGCGGCTCC |
| | | GGAACCGACTATACCTTTACAATCTCCTCTCTGCAGCCCGAGG |
| | | ATATCGCCACATACTTCTGTCTGCAGCACAGAAGCGGCTATAC |
| | | CTTTGGCCAGGGCACAAAGCTGGAGATCAAGCGTACG |
| SEQ ID NO: 451 | Light | DIQMTQSPSSLSASVGDRVTITCKSSQNIDKYLNWYQQKPGKAPK |
| | chain | LLIYNTNNLEAGVPSRFSGSGSGTDYTFTISSLQPEDIATYFCLQHR |
| | | SGYTFGQGTKLEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC |
| SEQ ID NO: 452 | DNA | GGATCCACCGGCGACATCCAGATGACCCAGTCCCCAAGCTCC |
| | Light | CTGAGCGCCTCCGTGGGCGACCGGGTGACCATCACATGCAAG |
| | Chain | TCTAGCCAGAACATCGATAAGTACCTGAATTGGTATCAGCAGAA |
| | | GCCCGGCAAGGCCCCTAAGCTGCTGATCTACAACACAAACAAT |
| | | CTGGAGGCCGGCGTGCCATCTCGGTTCTCTGGCAGCGGCTCC |
| | | GGAACCGACTATACCTTTACAATCTCCTCTCTGCAGCCCGAGG |
| | | ATATCGCCACATACTTCTGTCTGCAGCACAGAAGCGGCTATAC |
| | | CTTTGGCCAGGGCACAAAGCTGGAGATCAAGCGTACGCGTAC |
| | | GGTGGCTGCACCATCTGTCTTCATCTTCCCGCCATCTGATGAG |
| | | CAGTTGAAATCTGGAACTGCCTCTGTTGTGTGCCTGCTGAATAA |
| | | CTTCTATCCCCGCGAGGCCAAAGTACAGTGGAAGGTGGATAAC |
| | | GCCCTCCAATCGGGTAACTCCCAGGAGAGTGTCACAGAGCAG |
| | | GACAGCAAGGACAGCACCTACAGCCTCAGCAGCACCCTGACG |
| | | CTGAGCAAAGCAGACTACGAGAAACACAAAGTCTACGCCTGCG |
| | | AAGTCACCCATCAGGGCCTGAGCTCGCCCGTCACAAAGAGCTT |
| | | CAACCGCGGAGAGTGT |

### 8.2. Example 2: Binding of CD-3 Antibodies

Binding affinity interaction (KD) of the anti-CD3 antibodies was determined utilizing surface plasma resonance (SPR) technology. CD3 protein was immobilized onto a Series S CM5 sensor chip and antibody flowed over in 2 fold serial dilutions to assess binding utilizing the Biacore T200 instrument (GE Heathcare, Cat #28975001, Pittsburgh, Pa.). The KD was determined by fitting the plot with a 1:1 fit model (O'Shannessy et al. Anal. Biochem 1993; 212: 457-468; Karlsson, Fält J. Immunol. Methods. 1997; 200: 121-133). This SPR data is shown in FIGS. 4A-4D. This figure shows that NOV292 (FIG. 4A) and NOV123 (FIG. 4C) are efficient binders and comparable with the SP34 positive control in FIG. 4B. The SP1C antibody in FIG. 4D is a rat parental antibody that has not been humanized.

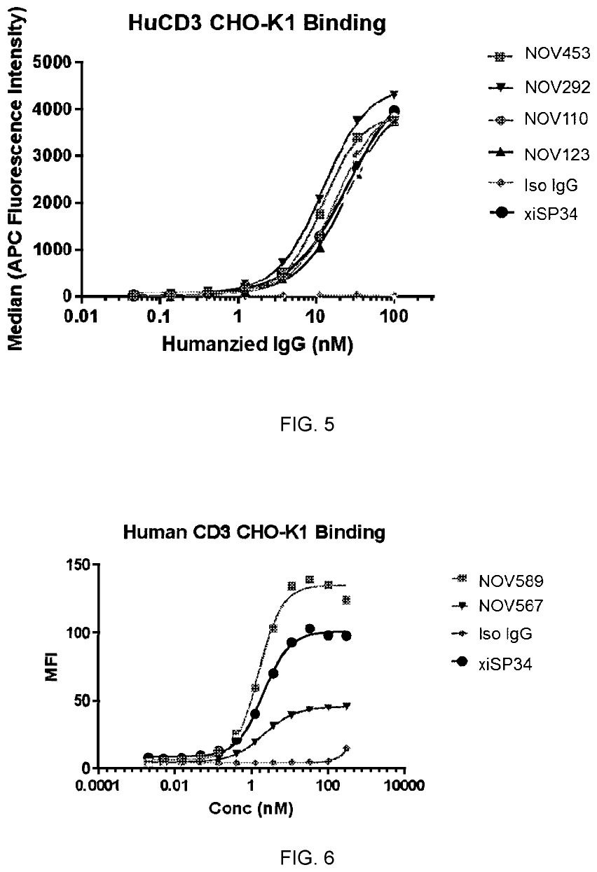

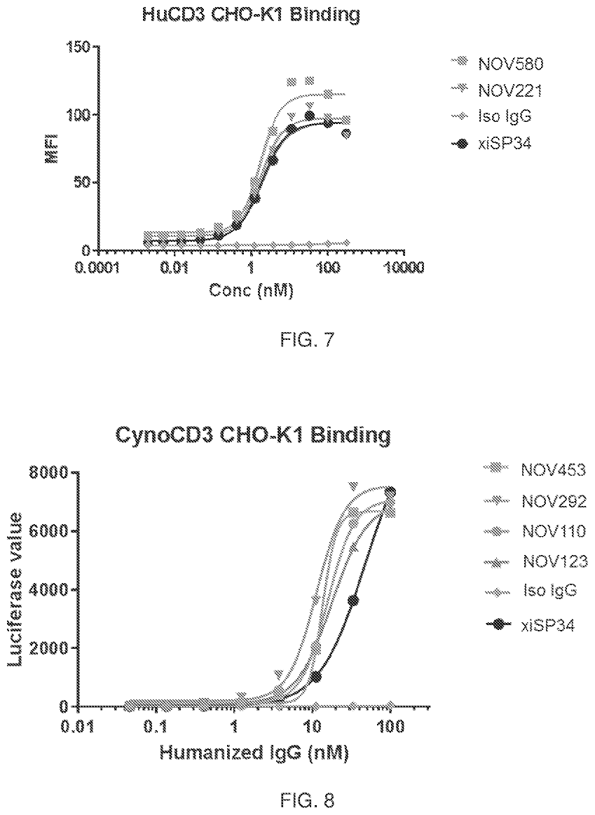

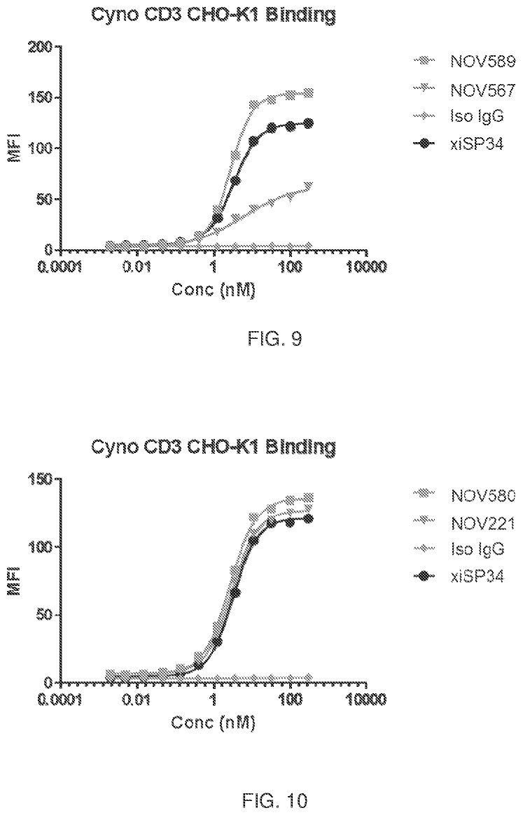

Binding activity (EC50) of the anti-CD3 antibodies with the human CD3 or cynomolgus monkey CD3 stably expressed in CHO cell lines were determined utilizing flow cytometry (FACS) technology. Human CD3 stably expressed CHO cell line (hCD3-CHO) was kept in RPMI 1640 culture medium (Gibco, Cat #11875119) plus 10% FBS (Gibco, Cat #10099141) and 1 mg/ml G418 (Gibco, Cat #10131035), while the cyno CD3 stably expressed CHO cell line (cynoCD3-CHO) was maintained in RPMI 1640 culture medium plus 10% FBS, 250 ug/ml Zeocin (Gibco, Cat #R25005) and 1 mg/ml G418. For FACS, the culture media was removed, and the cells were washed once with 1×PBS (Gibco, Cat #10099-041). The PBS was aspirated the cells were resuspended in 5 mM EDTA PBS. The cells were counted and diluted in 0.2% BSA PBS. The cells were transferred to U bottom 96 plates (1×106 per well). Cells were pelleted and re-suspended with serially diluted antibodies. An anti-huCD3 SP34 xiII IgG1-LALA antibody made in-house was used a positive control. Antibodies were incubated with the cells for 30 min, then 200 μl of cold PBS+1% FBS was added and cells pelleted. The cells were then counter stained with 50 μl of 1 μg/ml anti-human A647 detection antibody (Jacson ImmunoResearch, Cat #115-606-071) in cold PBS+1% FBS. The cells were incubated for 20 min at 4° C. and then 200 μl of cold PBS+1% FBS was added and cells were subjected to FACS analysis on a Guava 8™ instrument or BD Fortessa™ instrument. Original data were analyzed by Flowjo software, and this data is shown in FIGS. 5-10. This data shows that the antibodies disclosed herein bind to both human and cyno CD3 with values similar to the SP34 positive control.

### 8.3. Example 3: Activation of JNL Cells by Anti-CD3 Agonist Antibodies

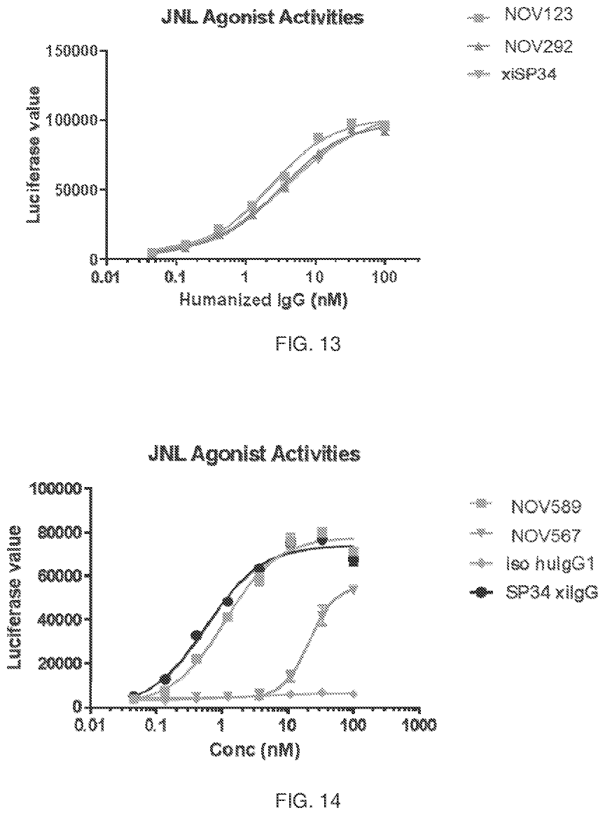

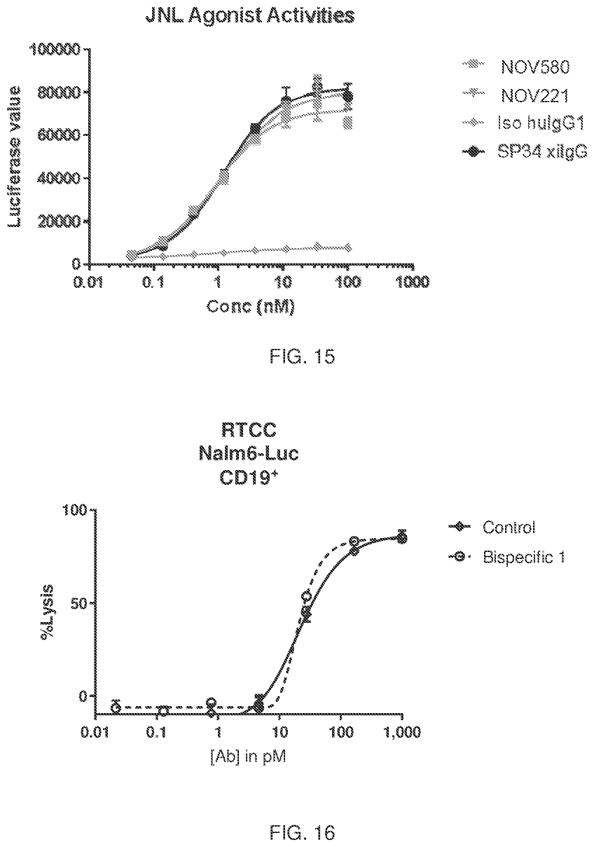

Jurkat NFAT Luciferase (JNL) cells were cultured in RPMI1640 medium plus 10% FBS, 2 mM Glutamax (Gibco, Cat #35050061) and 0.5 μg/ml Puromycin (Gibco, Cat #A1113802). Plates were coated with serial diluted antibodies and incubate at 37° C. for 1 hour. After incubation, the plates were washed with 200 μl wash buffer (PBS+1% FBS), then 100 μl of the prepared JNL cells (2.5×10⁵cells/mL) were added to all wells. The cells were incubated at 37° C., 5% CO2 for 14h-16h. The plates were removed from the incubator and 100 μl of One-glo reagent (Promega, Cat #E6120) was added. The reagent was incubated for 3 minutes at room temperature, then placed in a light sealed dark box for 10 minutes at room temperature. The samples were transferred to a solid white bottom tissue culture plate and data was analyzed using the Envision Plate Reader (Perkin Elmer, Cat #2105-0010, Bridgeville Pa.). The results are shown in FIGS. 11-15. This data demonstrates that the antibodies disclosed herein displayed the same activity in the JNL cell assay as SP34.

### 8.4. Example 4: Production of Anti-CD3-Anti-CD19 IgG1 Bispecific Antibodies in Knob-into-Holes Format

Gene synthesis for all constructs was performed and codon optimized for expression in mammalian cells. For bispecific constructs, anti-CD19 heavy chains were synthesized as fusions of the variable domains to constant human IgG1 domains containing mutations for the hole to facilitate heterodimerization as well as N297A Fc silencing mutation. Anti-CD19 light chain plasmids were also synthesized for expression. The anti-CD3 arm was produced as single chain fragment variable fused to constant human IgG1 domains containing mutations for the knob to facilitate heterodimerization as well as the N297A Fc silencing mutation. Bispecific antibodies were co-expressed transiently in HEK293 cells. The transfection was performed using PEI as the transfection reagent. For small scale (<5 L) transfections, cells were grown in shake flasks on an orbital shaker (115 rpm) in a humidified incubator (85%) at 5% CO2. Light and heavy chain plasmids for tumor antigen arms were combined with anti-CD3 plasmid with PEI at a final ratio of 1 DNA:3 PEI. 1 mg/L culture of plasmid was used for transfection at 2.0 million cells/ml. After 5 days of expression, the antibody was harvested by clarification of the media via centrifugation and filtration. Purification was performed via anti-CH1 affinity batch binding (CaptureSelect IgG-CH1 Affinity Matrix, Thermo-Fisher Scientific, Waltham, Mass., USA) or Protein A (rProteinA Sepharose, Fast flow, GE Healthcare, Uppsala, Sweden) batch binding using 1 ml resin/100 ml supernatant. The protein was allowed to bind for a minimum of 2 hours with gentle mixing, and the supernatant loaded onto a gravity filtration column. The resin was washed with 20-50 CV of PBS. Antibody was eluted with 20 CV of 50 mM citrate, 90 mM NaCl pH 3.2. 50 mM sucrose. The eluted IgG protein was adjusted to pH 5.5 with 1 M sodium citrate 50 mM sucrose. If the antibody contained aggregates, preparative size exclusion chromatography was performed using Hi Load 16/60 Superdex 200 grade column (GE Healthcare Life Sciences, Uppsala, Sweden) as a final polishing step. To confirm that the identity of the proteins expressed matched the predicted masses for the primary amino acid sequences from Seq ID No: 453-461 below, bispecific proteins were analyzed by high-performance liquid chromatography coupled to mass spectrometry.

TABLE 20

| | | |
|---|---|---|
| CD19-CD3 | | |
| bispecific control | | |
| SEQ ID NO: | CD19 light | EIVMTQSPATLSLSPGERATLSCRASQDISKYLNW |
| 453 | chain | YQQKPGQAPRLLIYHTSRLHSGIPARFSGSGSGTD |
| | | YTLTISSLQPEDFAVYFCQQGNTLPYTFGQGTKLEI |
| | | KRTVAAPSVFIFPPSDEQLKSGTASVVCLLNNFYP |
| | | REAKVQWKVDNALQSGNSQESVTEQDSKDSTYSL |
| | | SSTLTLSKADYEKHKVYACEVTHQGLSSPVTKSFN |
| | | RGEC |
| SEQ ID NO: | CD19 | QVQLQESGPGLVKPSETLSLTCTVSGVSLPDYGVS |
| 454 | heavy | WIRQPPGKGLEWIGVIWGSETTYYQSSLKSRVTIS |
| | chain | KDNSKNQVSLKLSSVTAADTAVYYCAKHYYYGGS |
| | | YAMDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGP |
| | | SVFLFPPKPKDTLMISRTPEVTCVVVDVSHEDPEV |
| | | KFNWYVDGVEVHNAKTKPREEQYASTYRVVSVLT |
| | | VLHQDWLNGKEYKCKVSNKALPAPIEKTISKAKGQ |
| | | PREPQVCTLPPSREEMTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PGK |
| SEQ ID NO: | CD3 scFv | EVQLVESGGGLVQPGGSLKLSCAASGFTFNTYAM |
| 455 | heavy | NWVRQASGKGLEWVGRIRSKYNNYATYYADSVK |
| | chain | DRFTISRDDSKSTLYLQMNSLKTEDTAVYYCVRHG |
| | | NFGNSYVSWFAYWGQGTLVTVSSGGGGSGGGG |
| | | SGGGGSGGGGSQAVVTQEPSLTVSPGGTVTLTC |
| | | RSSTGAVTTSNYANWVQQKPGQAPRGLIGGTNKR |
| | | APWTPARFSGSLLGDKAALTLSGAQPEDEAEYFC |
| | | ALWYSNLWVFGGGTKLTVLGGGGSDKTHTCPPC |
| | | PAPELLGGPSVFLFPPKPKDTLMISRTPEVTCVVVD |
| | | VSHEDPEVKFNWYVDGVEVHNAKTKPREEQYAST |
| | | YRVVSVLTVLHQDWLNGKEYKCKVSNKALPAPIEK |
| | | TISKAKGQPREPQVYTLPPCREEMTKNQVSLWCL |
| | | VKGFYPSDIAVEWESNGQPENNYKTTPPVLDSDG |
| | | SFFLYSKLTVDKSRWQQGNVFSCSVMHEALHNHY |
| | | TQKSLSLSPGK |
| CD19-CD3 | | |
| bispecific 1 | | |
| (Rat, sp10b) | | |
| SEQ ID NO: 453 | CD19 light | EIVMTQSPATLSLSPGERATLSCRASQDISKYLNW |
| | chain | YQQKPGQAPRLLIYHTSRLHSGIPARFSGSGSGTD |
| | | YTLTISSLQPEDFAVYFCQQGNTLPYTFGQGTKLEI |
| | | KRTVAAPSVFIFPPSDEQLKSGTASVVCLLNNFYP |
| | | REAKVQWKVDNALQSGNSQESVTEQDSKDSTYSL |
| | | SSTLTLSKADYEKHKVYACEVTHQGLSSPVTKSFN |
| | | RGEC |
| SEQ ID NO: 454 | CD19 | QVQLQESGPGLVKPSETLSLTCTVSGVSLPDYGVS |
| | heavy | WIRQPPGKGLEWIGVIWGSETTYYQSSLKSRVTIS |
| | chain | KDNSKNQVSLKLSSVTAADTAVYYCAKHYYYGGS |
| | | YAMDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGP |
| | | SVFLFPPKPKDTLMISRTPEVTCVVVDVSHEDPEV |
| | | KFNWYVDGVEVHNAKTKPREEQYASTYRVVSVLT |
| | | VLHQDWLNGKEYKCKVSNKALPAPIEKTISKAKGQ |
| | | PREPQVCTLPPSREEMTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PGK |
| SEQ ID NO: 456 | CD3 scFv | QVQLHQSGAELAKPGTSVNLSCKASGYTFTSYYIY |
| | heavy | WIKRRPGQGLEWIGYIYPGHDAIYYSENFKGKATF |
| | chain | TADTSSSTAYMLLGSLTPEDSAYYFCVRPNTMMA |
| | | PLAYWGQGTLVTVSSGGGGSGGGGSGGGGSGG |
| | | GGSVVVLTQTPVSLPVSLGGQASISCRSSQSLIYSI |
| | | GNTYLHWYLQKPGQSPQLLIYRVSNRFSGVPDRF |
| | | SGSGSGTDFTLKISRVEPEDLGDYYCFQSTHLPYT |
| | | FGAGTKLELKGGGGSGGGGSDKTHTCPPCPAPEL |
| | | LGGPSVFLFPPKPKDTLMISRTPEVTCVVVDVSHE |
| | | DPEVKFNWYVDGVEVHNAKTKPREEQYNSTYRVV |
| | | SVLTVLHQDWLNGKEYKCKVSNKALPAPIEKTISKA |
| | | KGQPREPQVYTLPPCREEMTKNQVSLWCLVKGFY |
| | | PSDIAVEWESNGQPENNYKTTPPVLDSDGSFFLYS |
| | | KLTVDKSRWQQGNVFSCSVMHEALHNRYTQKSLS |
| | | LSPGK |
| CD19-CD3 | | |
| bispecific 2 | | |
| (NOV110) | | |
| SEQ ID NO: 453 | CD19 light | EIVMTQSPATLSLSPGERATLSCRASQDISKYLNW |
| | chain | YQQKPGQAPRLLIYHTSRLHSGIPARFSGSGSGTD |
| | | YTLTISSLQPEDFAVYFCQQGNTLPYTFGQGTKLEI |
| | | KRTVAAPSVFIFPPSDEQLKSGTASVVCLLNNFYP |
| | | REAKVQWKVDNALQSGNSQESVTEQDSKDSTYSL |
| | | SSTLTLSKADYEKHKVYACEVTHQGLSSPVTKSFN |
| | | RGEC |
| SEQ ID NO: 454 | CD19 | QVQLQESGPGLVKPSETLSLTCTVSGVSLPDYGVS |
| | heavy | WIRQPPGKGLEWIGVIWGSETTYYQSSLKSRVTIS |
| | chain | KDNSKNQVSLKLSSVTAADTAVYYCAKHYYYGGS |
| | | YAMDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGP |
| | | SVFLFPPKPKDTLMISRTPEVTCVVVDVSHEDPEV |
| | | KFNWYVDGVEVHNAKTKPREEQYASTYRVVSVLT |
| | | VLHQDWLNGKEYKCKVSNKALPAPIEKTISKAKGQ |
| | | PREPQVCTLPPSREEMTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PGK |
| SEQ ID NO: 457 | CD3 scFv | QVQLVQSGAEVKKPGASVKVSCKASGYTFTSYYIY |
| | heavy | WVRQAPGQRLEWMGYIYPANGGIYYSEKFKGRVT |
| | chain | ITADTSAGTAYMELSSLRSEDTAVYYCARPVTMMA |
| | | PLVFWGQGTLVTVSSGGGGSGGGGSGGGGSGG |
| | | GGSDVVMTQSPLSLPVTLGQPASISCRSSQSLVYS |
| | | HGNTYLHWYQQRPGQSPRLLIYRVSNRFSGVPDR |
| | | FSGSGSGTDFTLKISRVEAEDVGVYYCFQSTHLPY |
| | | TFGQGTKLEIKGSDKTHTCPPCPAPELLGGPSVFL |
| | | FPPKPKDTLMISRTPEVTCVVVDVSHEDPEVKFNW |
| | | YVDGVEVHNAKTKPREEQYASTYRVVSVLTVLHQ |
| | | DWLNGKEYKCKVSNKALPAPIEKTISKAKGQPREP |
| | | QVYTLPPCREEMTKNQVSLWCLVKGFYPSDIAVE |
| | | WESNGQPENNYKTTPPVLDSDGSFFLYSKLTVDK |
| | | SRWQQGNVFSCSVMHEALHNRYTQKSLSLSPGK |
| CD19-CD3 | | |
| bispecific 3 | | |
| (NOV567) | | |
| SEQ ID NO: 453 | CD19 light | EIVMTQSPATLSLSPGERATLSCRASQDISKYLNW |
| | chain | YQQKPGQAPRLLIYHTSRLHSGIPARFSGSGSGTD |
| | | YTLTISSLQPEDFAVYFCQQGNTLPYTFGQGTKLEI |
| | | KRTVAAPSVFIFPPSDEQLKSGTASVVCLLNNFYP |
| | | REAKVQWKVDNALQSGNSQESVTEQDSKDSTYSL |
| | | SSTLTLSKADYEKHKVYACEVTHQGLSSPVTKSFN |
| | | RGEC |
| SEQ ID NO: 454 | CD19 | QVQLQESGPGLVKPSETLSLTCTVSGVSLPDYGVS |
| | heavy | WIRQPPGKGLEWIGVIWGSETTYYQSSLKSRVTIS |
| | chain | KDNSKNQVSLKLSSVTAADTAVYYCAKHYYYGGS |
| | | YAMDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGP |
| | | SVFLFPPKPKDTLMISRTPEVTCVVVDVSHEDPEV |
| | | KFNWYVDGVEVHNAKTKPREEQYASTYRVVSVLT |
| | | VLHQDWLNGKEYKCKVSNKALPAPIEKTISKAKGQ |
| | | PREPQVCTLPPSREEMTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PGK |
| SEQ ID NO: 458 | CD3 scFv | QVQLVESGGGVVQPGRSLRLSCAASGFAFRKYG |
| | heavy | MSWVRQAPGKGLEWVALIYYDSSKMNYADTVKG |
| | chain | RFTISRDNSKNTLYLQMNSLRAEDTAVYYCAALNS |
| | | EYDWGQGTMVTVSGGGGSGGGGSGGGGSGGG |
| | | GSDIQMTQSPSSLSASVGDRVTITCRGSQSIGNSL |
| | | NWYQQKPGKAPKRLIYSTSTLEYGVPSRFSGSGS |
| | | GTEYTLTISSLQPEDFATYYCLQYATYPYTFGQGT |
| | | KLEIKGSDKTHTCPPCPAPELLGGPSVFLFPPKPK |
| | | DTLMISRTPEVTCVVVDVSHEDPEVKFNWYVDGV |
| | | EVHNAKTKPREEQYASTYRVVSVLTVLHQDWLNG |
| | | KEYKCKVSNKALPAPIEKTISKAKGQPREPQVYTLP |
| | | PCREEMTKNQVSLWCLVKGFYPSDIAVEWESNGQ |
| | | PENNYKTTPPVLDSDGSFFLYSKLTVDKSRWQQG |
| | | NVFSCSVMHEALHNRYTQKSLSLSPGK |
| CD19-CD3 | | |
| bispecific 4 | | |
| (NOV221) | | |
| SEQ ID NO: 453 | CD19 light | EIVMTQSPATLSLSPGERATLSCRASQDISKYLNW |
| | chain | YQQKPGQAPRLLIYHTSRLHSGIPARFSGSGSGTD |
| | | YTLTISSLQPEDFAVYFCQQGNTLPYTFGQGTKLEI |
| | | KRTVAAPSVFIFPPSDEQLKSGTASVVCLLNNFYP |
| | | REAKVQWKVDNALQSGNSQESVTEQDSKDSTYSL |
| | | SSTLTLSKADYEKHKVYACEVTHQGLSSPVTKSFN |
| | | RGEC |
| SEQ ID NO: 454 | CD19 | QVQLQESGPGLVKPSETLSLTCTVSGVSLPDYGVS |
| | heavy | WIRQPPGKGLEWIGVIWGSETTYYQSSLKSRVTIS |
| | chain | KDNSKNQVSLKLSSVTAADTAVYYCAKHYYYGGS |
| | | YAMDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGP |
| | | SVFLFPPKPKDTLMISRTPEVTCVVVDVSHEDPEV |
| | | KFNWYVDGVEVHNAKTKPREEQYASTYRVVSVLT |
| | | VLHQDWLNGKEYKCKVSNKALPAPIEKTISKAKGQ |
| | | PREPQVCTLPPSREEMTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PGK |
| SEQ ID NO: 454 | CD3 scFv | QVQLQESGPGLVKPSETLSLTCTVSGVSLPDYGVS |
| | heavy | WIRQPPGKGLEWIGVIWGSETTYYQSSLKSRVTIS |
| | chain | KDNSKNQVSLKLSSVTAADTAVYYCAKHYYYGGS |
| | | YAMDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGP |
| | | SVFLFPPKPKDTLMISRTPEVTCVVVDVSHEDPEV |
| | | KFNWYVDGVEVHNAKTKPREEQYASTYRVVSVLT |
| | | VLHQDVVLNGKEYKCKVSNKALPAPIEKTISKAKGQ |
| | | PREPQVCTLPPSREEMTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PGK |
| CD19-CD3 | | |
| bispecific 5 | | |
| (NOV123) | | |
| SEQ ID NO: 453 | CD19 light | EIVMTQSPATLSLSPGERATLSCRASQDISKYLNW |
| | chain | YQQKPGQAPRLLIYHTSRLHSGIPARFSGSGSGTD |
| | | YTLTISSLQPEDFAVYFCQQGNTLPYTFGQGTKLEI |
| | | KRTVAAPSVFIFPPSDEQLKSGTASVVCLLNNFYP |
| | | REAKVQWKVDNALQSGNSQESVTEQDSKDSTYSL |
| | | SSTLTLSKADYEKHKVYACEVTHQGLSSPVTKSFN |
| | | RGEC |
| SEQ ID NO: 454 | CD19 | QVQLQESGPGLVKPSETLSLTCTVSGVSLPDYGVS |
| | heavy | WIRQPPGKGLEWIGVIWGSETTYYQSSLKSRVTIS |
| | chain | KDNSKNQVSLKLSSVTAADTAVYYCAKHYYYGGS |
| | | YAMDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGP |
| | | SVFLFPPKPKDTLMISRTPEVTCVVVDVSHEDPEV |
| | | KFNWYVDGVEVHNAKTKPREEQYASTYRVVSVLT |
| | | VLHQDWLNGKEYKCKVSNKALPAPIEKTISKAKGQ |
| | | PREPQVCTLPPSREEMTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PGK |
| SEQ ID NO: 460 | CD3 scFv | QVQLVQSGAEVKKPGASVKVSCKASGYTFTSYYIY |
| | heavy | WVRQAPGQRLEVVMGYIYPGHDAIYYSENFKGRVT |
| | chain | ITADTSASTAYMELSSLRSEDTAVYYCVRPNTMMA |
| | | PLAYWGQGTLVTVSSGGGGSGGGGSGGGGSGG |
| | | GGSDVVMTQSPLSLPVTLGQPASISCRSSQSLIYSI |
| | | GNTYLHWYQQRPGQSPRLLIYRVSNRFSGVPDRF |
| | | SGSGSGTDFTLKISRVEAEDVGVYYCFQSTHLPYT |
| | | FGQGTKLEIKGSDKTHTCPPCPAPELLGGPSVFLF |
| | | PPKPKDTLMISRTPEVTCVVVDVSHEDPEVKFNWY |
| | | VDGVEVHNAKTKPREEQYASTYRVVSVLTVLHQD |
| | | VVLNGKEYKCKVSNKALPAPIEKTISKAKGQPREPQ |
| | | VYTLPPCREEMTKNQVSLWCLVKGFYPSDIAVEW |
| | | ESNGQPENNYKTTPPVLDSDGSFFLYSKLTVDKSR |
| | | WQQGNVFSCSVMHEALHNRYTQKSLSLSPGK |
| CD19-CD3 | | |
| bispecific 6 | | |
| (NOV453) | | |
| SEQ ID NO: 453 | CD19 light | EIVMTQSPATLSLSPGERATLSCRASQDISKYLNW |
| | chain | YQQKPGQAPRLLIYHTSRLHSGIPARFSGSGSGTD |
| | | YTLTISSLQPEDFAVYFCQQGNTLPYTFGQGTKLEI |
| | | KRTVAAPSVFIFPPSDEQLKSGTASVVCLLNNFYP |
| | | REAKVQWKVDNALQSGNSQESVTEQDSKDSTYSL |
| | | SSTLTLSKADYEKHKVYACEVTHQGLSSPVTKSFN |
| | | RGEC |
| SEQ ID NO: 454 | CD19 | QVQLQESGPGLVKPSETLSLTCTVSGVSLPDYGVS |
| | heavy | WIRQPPGKGLEWIGVIWGSETTYYQSSLKSRVTIS |
| | chain | KDNSKNQVSLKLSSVTAADTAVYYCAKHYYYGGS |
| | | YAMDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGP |
| | | SVFLFPPKPKDTLMISRTPEVTCVVVDVSHEDPEV |
| | | KFNWYVDGVEVHNAKTKPREEQYASTYRVVSVLT |
| | | VLHQDWLNGKEYKCKVSNKALPAPIEKTISKAKGQ |
| | | PREPQVCTLPPSREEMTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PGK |
| SEQ ID NO: 461 CD3 scFv | | QVQLQESGPGLVKPSETLSLTCTVSGVSLPDYGVS |
| | heavy | WIRQPPGKGLEWIGVIWGSETTYYQSSLKSRVTIS |
| | chain | KDNSKNQVSLKLSSVTAADTAVYYCAKHYYYGGS |
| | | YAMDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGP |
| | | SVFLFPPKPKDTLMISRTPEVTCVVVDVSHEDPEV |
| | | KFNWYVDGVEVHNAKTKPREEQYASTYRVVSVLT |
| | | VLHQDWLNGKEYKCKVSNKALPAPIEKTISKAKGQ |
| | | PREPQVCTLPPSREEMTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PGK |

### 8.5. Example 5: In Vitro Functional Activity of Anti-CD19-Anti-CD3 Bispecific Antibodies

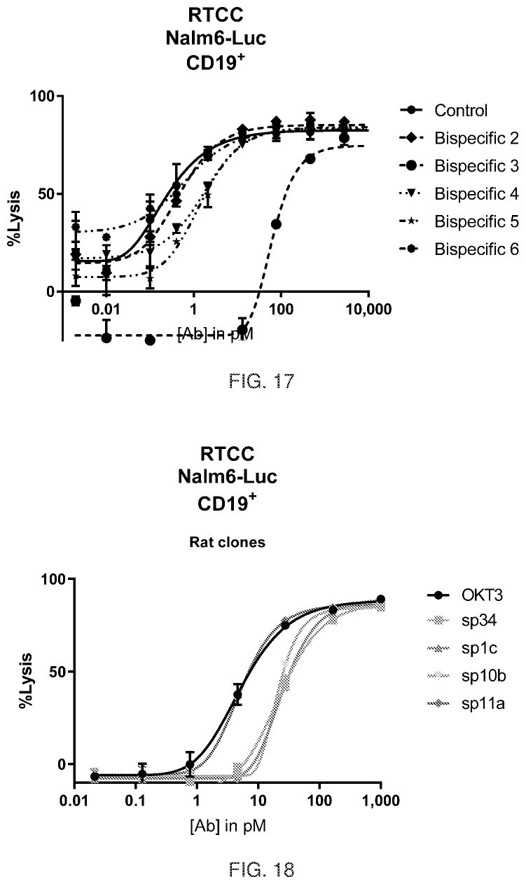

In vitro functional activity was evaluated with a luciferase based cytotoxicity assay using the ALL cell line NALM-6 (DSMZ, Braunschwieg, Germany) which was transduced to stably express luciferase. Human T Cells isolated from cryopreserved PBMCs then expanded for 10 days with CD3/CD28 beads (ThermoFisher Scientific, Grand Island, N.Y., USA) were co-cultured at an effector:target ratio of 5:1 with the NALM-6 target cells. Bispecific antibodies were added at various concentrations and incubated for 20 hours after which ONE-Glo Luciferase Assay substrate (Promega, Madison, Wis., USA) was added. Luminescence was measured for treated and untreated (to provide maximal luminescence signal) wells and specific lysis (%) was determined as 100−(sample luminescence/average maximal luminescence)*100. The data is shown graphically in FIGS. 16-17. FIGS. 16-17 demonstrate that a bispecific CD19/CD3 antibody as disclosed herein is efficacious in a Redirected T-cell Cytotoxicity (RTCC) assay. As shown in FIG. 17, several anti-CD3 arms were of better or equal activity to the SP34 control. Bispecific 1 in FIG. 16 is a non-humanized, rat parental antibody.

### 8.6. Example 6: Anti-CD3-Anti-CD19 Bispecifc Antibodies: Rat Clones

Bispecific antibodies in the format of FIG. 1C having an anti-CD3 scFv and anti-CD19 Fab were constructed with VH and VL sequences of rat antibodies of Example 1 and expressed using the expression procedures described in Example 4. Constructs were characterized by measuring CD3+ T cell affinity by FACS and activity in a RTCC assay.

FACS was performed using human T cells isolated from cryopreserved PBMCs that were expanded for 10 days with CD3/CD28 beads (ThermoFisher Scientific, Grand Island, N.Y., USA). Cells were plated at 250,000 cells/well, 180 uL/well in cold FACS buffer (PBS/10% Fetal Bovine Serum). Constructs were prepared at an initial concentration of 1 μM and serially diluted 1:4. 20 μL of each construct was added to the respective well for a final volume of 200 μL and a final starting concentration 100 nM. Cells were incubated on ice for one hour. After washing and resuspension with cold FACS buffer, 1 μg of goat anti-human IgG Fc-specific antibody (Jackson Immunoresearch, 109-605-005) was added to each well. The secondary antibody was incubated for one hour on ice. Cells were then washed and ran on the cytometer (Beckman Coulter Cytoflex, B53011). Data was analyzed on GraphPad Prism using 5-parameter logistic fit.

RTCC was performed as described in Example 5.

RTCC assay results for constructs having VH and VL sequences of rat antibodies sp1c, sp10b (which is the parental antibody of NOV123), and sp11a (which is the parental antibody of NOV292) are shown in FIG. 18. Results of the CD3+ T cell affinity assay for those constructs are shown in FIG. 19.

### 8.7. Example 7: Anti-CD19-Anti-CD3 Bispecific Antibodies: Sp11a Variants

A series of bispecific antibodies having a CD3 binding arm based on sp11a (which is the parental antibody of NOV292) in scFv format and a CD19 binding arm in Fab format were produced in the format of FIG. 1C and expressed using the expression procedures described in Example 4, with the aim of producing bispecific antibodies with improved expression and/or altered function. Constructs having mutations that mitigate post translational modifications (referred to as “PTM” variants), constructs having backmutations (referred to as “bkm” variants), and constructs having modifications in the “WW” residues of CDR-H3 were produced.

Specifically, PTM variants were produced by modifying the “NG” site in CDR-H1 and the “DG” site in CDR-L1, as these are PTM sites. The “NG” site is a deamidation site and “DG” is a D isomerization site. Without being bound by theory, it is believed that modification at PTM sites can affect the activity of the molecule, especially when the modification site is in the CDRs, and it is believed that these modifications can also affect the folding of the scFv and, as a result, its quality and yield. Accordingly, these sites were modified.

For the bkm variants, backmutation of vernier residues was performed with the aim of creating constructs with a diversity of affinities.

In sp11a, HCDR3 has a highly hydrophobic patch provided by the sequence “FWW.” Hydrophylic residues threonine and serine were chosen to break the hydrophobic patch. Without being bound by theory, it is believed that by making the sequence more hydrophilic, the propensity for the molecule to aggregate is reduced. In addition, again without being bound by theory, it is believed that the folding of the protein to form a scFv is less disrupted without the hydrophobic patch. Tyrosine was also included in the series due to its aromatic nature that is akin to tryptophan.

Constructs were analyzed for expression and activity in a RTCC assay (as described in Example 5). Polypeptide sequences of the constructs are shown in Table 21.

TABLE 21

| Construct | | SEQ |
|---|---|---|
| (polypeptide | | ID |
| chain) | Amino Acid Sequence | NO: |
| All | QVQLQESGPGLVKPSETLSLTCTVSGVSLPDYGVSWIRQPPGKGLEWIGVI | 516 |
| Constructs of | WGSETTYYQSSLKSRVTISKDNSKNQVSLKLSSVTAADTAVYYCAKHYYYG | |
| Example | GSYAMDYWGQGTLVTVSSASTKGPSVFPLAPSSKSTSGGTAALGCLVKDY | |
| (CD19 heavy | FPEPVTVSWNSGALTSGVHTFPAVLQSSGLYSLSSVVTVPSSSLGTQTYICN | |
| chain) | VNHKPSNTKVDKRVEPKSCDKTHTCPPCPAPELLGGPSVFLFPPKPKDTLM | |
| | ISRTPEVTCVVVDVSHEDPEVKFNWYVDGVEVHNAKTKPREEQYASTYRVV | |
| | SVLTVLHQDWLNGKEYKCKVSNKALPAPIEKTISKAKGQPREPQVCTLPPSR | |
| | EEMTKNQVSLSCAVKGFYPSDIAVEVVESNGQPENNYKTTPPVLDSDGSFFL | |
| | VSKLTVDKSRWQQGNVFSCSVMHEALHNHYTQKSLSLSPGK | |
| All | EIVMTQSPATLSLSPGERATLSCRASQDISKYLNWYQQKPGQAPRLLIYHTS | 517 |
| Constructs of | RLHSGIPARFSGSGSGTDYTLTISSLQPEDFAVYFCQQGNTLPYTFGQGTKL | |
| Example | EIKRTVAAPSVFIFPPSDEQLKSGTASVVCLLNNFYPREAKVQVVKVDNALQS | |
| (CD19 light | GNSQESVTEQDSKDSTYSLSSTLTLSKADYEKHKVYACEVTHQGLSSPVTK | |
| chain) | SFNRGEC | |
| CD3_sp11a_ | QVQLVESGGGVVQPGRSLRLSCAASGFTFSKNGMHWVRQAPGKGLEVVVA | 515 |
| bkm1 | MIYYDSSKMYYADTVKGRFTISRDNSKNTLYLQMNSLRAEDTAVYYCASFW | |
| (CD3 scFv | WDLDFDHWGQGTMVTVSSGGGGSGGGGSGGGGSGGGGSDIVMTQTPL | |
| heavy chain) | SSPVTLGQPASISCRSSQSLVRSDGTTYFNWLQQRPGQPPRLLIYRVSNRF | |
| | SGVPDRFSGSGAGTDFTLKISRVEAEDVGVYYCLQSSHFPVVTFGGGTKVEI | |
| | KGSDKTHTCPPCPAPELLGGPSVFLFPPKPKDTLMISRTPEVTCVVVDVSHE | |
| | DPEVKFNWYVDGVEVHNAKTKPREEQYASTYRVVSVLTVLHQDVVLNGKEY | |
| | KCKVSNKALPAPIEKTISKAKGQPREPQVYTLPPCREEMTKNQVSLWCLVK | |
| | GFYPSDIAVEVVESNGQPENNYKTTPPVLDSDGSFFLYSKLTVDKSRWQQG | |
| | NVFSCSVMHEALHNRYTQKSLSLSPGK | |
| CD3_SP11a_ | QVQLVESGGGVVQPGRSLRLSCAASGFTFSKNGMHWVRQAPGKGLEWVA | 518 |
| bkm2 | MIYYDSSKMYYADTVKGRFTISRDNSKNTLYLQMNSLRAEDTAVYYCAKFW | |
| (CD3 scFv | WDLDFDHWGQGTMVTVSSGGGGSGGGGSGGGGSGGGGSDIVMTQTPL | |
| heavy chain) | SSPVTLGQPASISCRSSQSLVRSDGTTYFNWYQQRPGQPPRLLIYRVSNRF | |
| | SGVPDRFSGSGAGTDFTLKISRVEAEDVGVYYCLQSSHFPWTFGGGTKVEI | |
| | KGGGSDKTHTCPPCPAPELLGGPSVFLFPPKPKDTLMISRTPEVTCVVVDV | |
| | SHEDPEVKFNWYVDGVEVHNAKTKPREEQYASTYRVVSVLTVLHQDWLNG | |
| | KEYKCKVSNKALPAPIEKTISKAKGQPREPQVYTLPPCREEMTKNQVSLWC | |
| | LVKGFYPSDIAVEWESNGQPENNYKTTPPVLDSDGSFFLYSKLTVDKSRWQ | |
| | QGNVFSCSVMHEALHNRYTQKSLSLSPGK | |
| CD3_sp11a_ | QVQLVESGGGVVQPGRSLRLSCAASGFTFSKNGMHWVRQAPGKGLEWVA | 519 |
| hz0 | MIYYDSSKMYYADTVKGRFTISRDNSKNTLYLQMNSLRAEDTAVYYCAKFW | |
| (CD3 scFv | WDLDFDHWGQGTMVTVSSGGGGSGGGGSGGGGSGGGGSDIVMTQTPL | |
| heavy chain) | SSPVTLGQPASISCRSSQSLVRSDGTTYFNWLQQRPGQPPRLLIYRVSNRF | |
| | SGVPDRFSGSGAGTDFTLKISRVEAEDVGVYYCLQSSHFPWTFGGGTKVEI | |
| | KGSDKTHTCPPCPAPELLGGPSVFLFPPKPKDTLMISRTPEVTCVVVDVSHE | |
| | DPEVKFNWYVDGVEVHNAKTKPREEQYASTYRVVSVLTVLHQDVVLNGKEY | |
| | KCKVSNKALPAPIEKTISKAKGQPREPQVYTLPPCREEMTKNQVSLWCLVK | |
| | GFYPSDIAVEWESNGQPENNYKTTPPVLDSDGSFFLYSKLTVDKSRWQQG | |
| | NVFSCSVMHEALHNRYTQKSLSLSPGK | |
| CD3_SP11A_ | QVQLVESGGGVVQPGRSLRLSCAASGFTFSKNGMHWVRQAPGKGLEWVA | 520 |
| HZ1 | MIYYDSSKMYYADTVKGRFTISRDNSKNTLYLQMNSLRAEDTAVYYCASFW | |
| (CD3 scFv | WDLDFDHWGQGTMVTVSSGGGGSGGGGSGGGGSGGGGSDIVMTQTPL | |
| heavy chain) | SSPVTLGQPASISCRSSQSLVRSDGTTYFNWYQQRPGQPPRLLIYRVSNRF | |
| | SGVPDRFSGSGAGTDFTLKISRVEAEDVGVYYCLQSSHGSDKTHTCPPCPA | |
| | PELLGGPSVFLFPPKPKDTLMISRTPEVTCVVVDVSHEDPEVKFNWYVDGV | |
| | EVHNAKTKPREEQYASTYRVVSVLTVLHQDVVLNGKEYKCKVSNKALPAPIE | |
| | KTISKAKGQPREPQVYTLPPCREEMTKNQVSLWCLVKGFYPSDIAVEVVESN | |
| | GQPENNYKTTPPVLDSDGSFFLYSKLTVDKSRWQQGNVFSCSVMHEALHN | |
| | RYTQKSLSLSPGK | |
| CD3_sp11a_ | QVQLVESGGGVVQPGRSLRLSCAASGFTFSKQGMHWVRQAPGKGLEWV | 521 |
| sansPTM_hz | AMIYYDSSKMYYADTVKGRFTISRDNSKNTLYLQMNSLRAEDTAVYYCASF | |
| 1 | VWVDLDFDHWGQGTMVTVSSGGGGSGGGGSGGGGSGGGGSDIVMTQTP | |
| (CD3 scFv | LSSPVTLGQPASISCRSSQSLVRSEGTTYFNWYQQRPGQPPRLLIYRVSNR | |
| heavy chain) | FSGVPDRFSGSGAGTDFTLKISRVEAEDVGVYYCLQSSHFPWTFGGGTKV | |
| | EIKGSDKTHTCPPCPAPELLGGPSVFLFPPKPKDTLMISRTPEVTCVVVDVS | |
| | HEDPEVKFNWYVDGVEVHNAKTKPREEQYASTYRVVSVLTVLHQDVVLNGK | |
| | EYKCKVSNKALPAPIEKTISKAKGQPREPQVYTLPPCREEMTKNQVSLWCL | |
| | VKGFYPSDIAVEWESNGQPENNYKTTPPVLDSDGSFFLYSKLTVDKSRWQ | |
| | QGNVFSCSVMHEALHNRYTQKSLSLSPGK | |
| CD3_sp11a_ | EVKLVESGGDLVQPGDSLTLSCVASGFTFSKQGMHWIRQAPKKGLEWIAMI | 522 |
| sansPTM_rat | YYDSSKMYYADTVKGRFTISRDNSKNTLYLEMNSLRSEDTAMYYCASFVWV | |
| (CD3 scFv | DLDFDHWGQGVMVTVSSGGGGSGGGGSGGGGSGGGGSDILVTQTPVSL | |
| heavy chain) | PVSLGGHVSISCRSSQSLVRSEGTTYFNWYLQKPGQSPQLLIYRVSNRFSG | |
| | VPDRFSGSGSGTDFTLKISRVEPEDLGVYYCLQSSHFPWTFGGGTKLELKG | |
| | GGGSDKTHTCPPCPAPELLGGPSVFLFPPKPKDTLMISRTPEVTCVVVDVS | |
| | HEDPEVKFNWYVDGVEVHNAKTKPREEQYASTYRVVSVLTVLHQDWLNGK | |
| | EYKCKVSNKALPAPIEKTISKAKGQPREPQVYTLPPCREEMTKNQVSLWCL | |
| | VKGFYPSDIAVEWESNGQPENNYKTTPPVLDSDGSFFLYSKLTVDKSRWQ | |
| | QGNVFSCSVMHEALHNRYTQKSLSLSPGK | |
| CD3_sp11a_ | QVQLVESGGGVVQPGRSLRLSCAASGFTFSKNGMHVVVRQAPGKGLEWVA | 523 |
| VHVL_YY | MIYYDSSKMYYADTVKGRFTISRDNSKNTLYLQMNSLRAEDTAVYYCASFY | |
| (CD3 scFv | YDLDFDHWGQGTMVTVSSGGGGSGGGGSGGGGSGGGGSDIVMTQTPLS | |
| heavy chain) | SPVTLGQPASISCRSSQSLVRSDGTTYFNWYQQRPGQPPRLLIYRVSNRFS | |
| | GVPDRFSGSGAGTDFTLKISRVEAEDVGVYYCLQSSHFPWTFGGGTKVEIK | |
| | GGGSDKTHTCPPCPAPELLGGPSVFLFPPKPKDTLMISRTPEVTCVVVDVS | |
| | HEDPEVKFNWYVDGVEVHNAKTKPREEQYASTYRVVSVLTVLHQDWLNGK | |
| | EYKCKVSNKALPAPIEKTISKAKGQPREPQVYTLPPCREEMTKNQVSLWCL | |
| | VKGFYPSDIAVEWESNGQPENNYKTTPPVLDSDGSFFLYSKLTVDKSRWQ | |
| | QGNVFSCSVMHEALHNHYTQKSLSLSPGK | |
| CD3_SP11A_ | QVQLVESGGGVVQPGRSLRLSCAASGFTFSKNGMHWVRQAPGKGLEWVA | 524 |
| VHVL_SS | MIYYDSSKMYYADTVKGRFTISRDNSKNTLYLQMNSLRAEDTAVYYCASFS | |
| (CD3 scFv | SDLDFDHWGQGTMVTVSSGGGGSGGGGSGGGGSGGGGSDIVMTQTPLS | |
| heavy chain) | SPVTLGQPASISCRSSQSLVRSDGTTYFNWYQQRPGQPPRLLIYRVSNRFS | |
| | GVPDRFSGSGAGTDFTLKISRVEAEDVGVYYCLQSSHFPVVTFGGGTKVEIK | |
| | GGGSDKTHTCPPCPAPELLGGPSVFLFPPKPKDTLMISRTPEVTCVVVDVS | |
| | HEDPEVKFNWYVDGVEVHNAKTKPREEQYASTYRVVSVLTVLHQDWLNGK | |
| | EYKCKVSNKALPAPIEKTISKAKGQPREPQVYTLPPCREEMTKNQVSLWCL | |
| | VKGFYPSDIAVEWESNGQPENNYKTTPPVLDSDGSFFLYSKLTVDKSRWQ | |
| | QGNVFSCSVMHEALHNHYTQKSLSLSPGK | |
| CD3_SP11A_ | QVQLVESGGGVVQPGRSLRLSCAASGFTFSKNGMHVVVRQAPGKGLEWVA | 525 |
| VHVL_WS | MIYYDSSKMYYADTVKGRFTISRDNSKNTLYLQMNSLRAEDTAVYYCASFW | |
| (CD3 scFv | SDLDFDHWGQGTMVTVSSGGGGSGGGGSGGGGSGGGGSDIVMTQTPLS | |
| heavy chain) | SPVTLGQPASISCRSSQSLVRSDGTTYFNWYQQRPGQPPRLLIYRVSNRFS | |
| | GVPDRFSGSGAGTDFTLKISRVEAEDVGVYYCLQSSHFPWTFGGGTKVEIK | |
| | GGGSDKTHTCPPCPAPELLGGPSVFLFPPKPKDTLMISRTPEVTCVVVDVS | |
| | HEDPEVKFNWYVDGVEVHNAKTKPREEQYASTYRVVSVLTVLHQDVVLNGK | |
| | EYKCKVSNKALPAPIEKTISKAKGQPREPQVYTLPPCREEMTKNQVSLWCL | |
| | VKGFYPSDIAVEWESNGQPENNYKTTPPVLDSDGSFFLYSKLTVDKSRWQ | |
| | QGNVFSCSVMHEALHNHYTQKSLSLSPGK | |
| CD3_sp11a_ | QVQLVESGGGVVQPGRSLRLSCAASGFTFSKNGMHWVRQAPGKGLEWVA | 526 |
| VHVL_SW | MIYYDSSKMYYADTVKGRFTISRDNSKNTLYLQMNSLRAEDTAVYYCASFS | |
| (CD3 scFv | WDLDFDHWGQGTMVTVSSGGGGSGGGGSGGGGSGGGGSDIVMTQTPL | |
| heavy chain) | SSPVTLGQPASISCRSSQSLVRSDGTTYFNWYQQRPGQPPRLLIYRVSNRF | |
| | SGVPDRFSGSGAGTDFTLKISRVEAEDVGVYYCLQSSHFPVVTFGGGTKVEI | |
| | KGGGSDKTHTCPPCPAPELLGGPSVFLFPPKPKDTLMISRTPEVTCVVVDV | |
| | SHEDPEVKFNWYVDGVEVHNAKTKPREEQYASTYRVVSVLTVLHQDWLNG | |
| | KEYKCKVSNKALPAPIEKTISKAKGQPREPQVYTLPPCREEMTKNQVSLWC | |
| | LVKGFYPSDIAVEWESNGQPENNYKTTPPVLDSDGSFFLYSKLTVDKSRWQ | |
| | QGNVFSCSVMHEALHNHYTQKSLSLSPGK | |
| CD3_SP11A_ | QVQLVESGGGVVQPGRSLRLSCAASGFTFSKNGMHWVRQAPGKGLEWVA | 527 |
| VHVL_TT | MIYYDSSKMYYADTVKGRFTISRDNSKNTLYLQMNSLRAEDTAVYYCASFTT | |
| (CD3 scFv | DLDFDHWGQGTMVTVSSGGGGSGGGGSGGGGSGGGGSDIVMTQTPLSS | |
| heavy chain) | PVTLGQPASISCRSSQSLVRSDGTTYFNWYQQRPGQPPRLLIYRVSNRFSG | |
| | VPDRFSGSGAGTDFTLKISRVEAEDVGVYYCLQSSHFPWTFGGGTKVEIKG | |
| | GGSDKTHTCPPCPAPELLGGPSVFLFPPKPKDTLMISRTPEVTCVVVDVSH | |
| | EDPEVKFNWYVDGVEVHNAKTKPREEQYASTYRVVSVLTVLHQDWLNGKE | |
| | YKCKVSNKALPAPIEKTISKAKGQPREPQVYTLPPCREEMTKNQVSLWCLV | |
| | KGFYPSDIAVEWESNGQPENNYKTTPPVLDSDGSFFLYSKLTVDKSRWQQ | |
| | GNVFSCSVMHEALHNHYTQKSLSLSPGK | |
| CD3_SP11A_ | QVQLVESGGGVVQPGRSLRLSCAASGFTFSKNGMHWVRQAPGKGLEWVA | 528 |
| VHVL_TW | MIYYDSSKMYYADTVKGRFTISRDNSKNTLYLQMNSLRAEDTAVYYCASFT | |
| (CD3 scFv | WDLDFDHWGQGTMVTVSSGGGGSGGGGSGGGGSGGGGSDIVMTQTPL | |
| heavy chain) | SSPVTLGQPASISCRSSQSLVRSDGTTYFNWYQQRPGQPPRLLIYRVSNRF | |
| | SGVPDRFSGSGAGTDFTLKISRVEAEDVGVYYCLQSSHFPVVTFGGGTKVEI | |
| | KGGGSDKTHTCPPCPAPELLGGPSVFLFPPKPKDTLMISRTPEVTCVVVDV | |
| | SHEDPEVKFNVVYVDGVEVHNAKTKPREEQYASTYRVVSVLTVLHQDWLNG | |
| | KEYKCKVSNKALPAPIEKTISKAKGQPREPQVYTLPPCREEMTKNQVSLWC | |
| | LVKGFYPSDIAVEWESNGQPENNYKTTPPVLDSDGSFFLYSKLTVDKSRWQ | |
| | QGNVFSCSVMHEALHNHYTQKSLSLSPGK | |
| CD3_SP11A_ | QVQLVESGGGVVQPGRSLRLSCAASGFTFSKNGMHWVRQAPGKGLEWVA | 529 |
| VHVL_VVT | MIYYDSSKMYYADTVKGRFTISRDNSKNTLYLQMNSLRAEDTAVYYCASFW | |
| (CD3 scFv | TDLDFDHWGQGTMVTVSSGGGGSGGGGSGGGGSGGGGSDIVMTQTPLS | |
| heavy chain) | SPVTLGQPASISCRSSQSLVRSDGTTYFNWYQQRPGQPPRLLIYRVSNRFS | |
| | GVPDRFSGSGAGTDFTLKISRVEAEDVGVYYCLQSSHFPVVTFGGGTKVEIK | |
| | GGGSDKTHTCPPCPAPELLGGPSVFLFPPKPKDTLMISRTPEVTCVVVDVS | |
| | HEDPEVKFNWYVDGVEVHNAKTKPREEQYASTYRVVSVLTVLHQDVVLNGK | |
| | EYKCKVSNKALPAPIEKTISKAKGQPREPQVYTLPPCREEMTKNQVSLWCL | |
| | VKGFYPSDIAVEWESNGQPENNYKTTPPVLDSDGSFFLYSKLTVDKSRWQ | |
| | QGNVFSCSVMHEALHNHYTQKSLSLSPGK | |

Results of the RTCC assay are shown in FIG. 20 and FIG. 21.

PTM mitigation improved both protein production and potentcy in the RTCC assay (FIG. 19). Backmutations improved production, but caused a loss in function. Mutations of the “WW” residues of CDR-H3 revealed that the identity of the amino acids at the WW positions impacts both protein production and potency. Each W position yields its own level of influence, with the SW variant providing the greatest boost in function. A summary of the expression and function data for several constructs is shown in Table 22.

TABLE 22

| | | | Expression | | |
|---|---|---|---|---|---|
| Variant | VH | VL | level | | Function |
| CD3_sp11a_rat | Rat | Rat | 4 | mg/L | Compable |
| | | | | | to sp34 |
| CD3_sp11a_Hz0 | Hz0 | Hz0 | None | | N/A |
| CD3_sp11a_bkm1 | Hz1 | Hz0 | 23 | mg/L | Worse than |
| | | | | | sp34 |
| CD3_sp11a_bkm2 | Hz0 | Hz1 | 9 | mg/L | Worse than |
| | | | | | sp34 |
| CD3_sp11a_Hz1 | Hz1 | Hz1 | None | | N/A |
| CD3_sp11a_sansPTM | Hz1 - NG | Hz1 - DG | 4 | mg/L | Worse than |
| | (deamidation) | (D isomerization) | | | sp34 |
| CD3_sp11a_sansPTM_rat | Rat - NG | Rat-NG | 20 | mg/mL | Better than |
| | | | | | sp34 |
| CD3_sp11a_VHVL_SW | hz1 WS | hz1 | 20 | mg/mL | Better than |
| | | | | | sp34 |

### 8.8. Example 8: Anti-CD19-Anti-CD3 Bispecific Antibodies: Sp9a Variants

A series of anti-CD19-anti-CD3 bispecific antibodies having a CD3 binding arm based on sp9a (which is the parental antibody of NOV229) in different frameworks were produced in the format of FIG. 1E and expressed as in Example 4 to explore the effect of framework choice on a construct's activity and stability. The orientation of the scFv was also manipulated from VHVL to VLVH to assess its effect on the construct's potency. Without being bound by theory, it is believed that by changing the orientation, the CDRs are exposed to the environment in a different manner, which, again without being bound by theory, is believed to affect activity. Constructs were analyzed for expression, activity in a RTCC assay (as described in Example 5) and binding to CD3+ T cells (as described in Example 6). Polypeptide sequences of the constructs are shown in Table 23.

TABLE 23

| Construct | | SEQ |
|---|---|---|
| (polypeptide | | ID |
| chain) | Amino Acid Sequence | NO: |
| All | DKTHTCPPCPAPELLGGPSVFLFPPKPKDTLMISRTPEVTCVVVAVSHEDPE | 540 |
| Constructs of | VKFNWYVDGVEVHNAKTKPREEQYASTYRVVSVLTVLHQDWLNGKEYKCK | |
| Example | VSNKALAAPIEKTISKAKGQPREPQVYTLPPCREEMTKNQVSLWCLVKGFY | |
| (Fc chain) | PSDIAVEWESNGQPENNYKTTPPVLDSDGSFFLYSKLTVDKSRWQQGNVF | |
| | SCSVMHEALHNRYTQKSLSLSPGK | |
| All | EIVMTQSPATLSLSPGERATLSCRASQDISKYLNWYQQKPGQAPRLLIYHTS | 517 |
| Constructs of | RLHSGIPARFSGSGSGTDYTLTISSLQPEDFAVYFCQQGNTLPYTFGQGTKL | |
| Example | EIKRTVAAPSVFIFPPSDEQLKSGTASVVCLLNNFYPREAKVQWKVDNALQS | |
| (CD19 light | GNSQESVTEQDSKDSTYSLSSTLTLSKADYEKHKVYACEVTHQGLSSPVTK | |
| chain) | SFNRGEC | |
| CD3_sp9aF | EVQLVQSGAEVKKPGESLKISCKASGYSFTNYWMNWVRQMPGKGLEVVMG | 534 |
| W1_VL_VH_ | MIHPSDSEIRLNQKFQGQVTLSVDKSIGTAYMQWSSLKASDTAMYYCSRVVY | |
| S56G (CD19 | YLSSPMDYWGQGTTVTVSSASTKGPSVFPLAPSSKSTSGGTAALGCLVKD | |
| Heavy CD3 | YFPEPVTVSWNSGALTSGVHTFPAVLQSSGLYSLSSVVTVPSSSLGTQTYIC | |
| scFv chain) | NVNHKPSNTKVDKRVEPKSCGGGGSGGGGSEIVMTQSPSTLSASVGDRVII | |
| | TCKASQNINNYLNWYQQKPGKAPKLLIYNTDHLQAGVPSRFSGSGSGAEFT | |
| | LTISSLQPDDFATYYCLQHRSRYTFGQGTKLTVLGGGGGSGGGGSGGGGS | |
| | GGGGSEVQLVESGGGLVQPGGSLRLSCAASGFSLTTYNVHWVRQAPGKG | |
| | LEWVGRMRYSGDTSFNAALTSRFTISRDNSKNTLYLQMNSLRAEDTAVYYC | |
| | ASDPMYIPNYAYGVMNAWGQGTLVTVSSGGGSDKTHTCPPCPAPELLGGP | |
| | SVFLFPPKPKDTLMISRTPEVTCVVVAVSHEDPEVKFNWYVDGVEVHNAKT | |
| | KPREEQYASTYRVVSVLTVLHQDWLNGKEYKCKVSNKALAAPIEKTISKAKG | |
| | QPREPQVCTLPPSREEMTKNQVSLSCAVKGFYPSDIAVEWESNGQPENNY | |
| | KTTPPVLDSDGSFFLVSKLTVDKSRWQQGNVFSCSVMHEALHNHYTQKSL | |
| | SLSPGK | |
| CD3_SP9AF | EVQLVQSGAEVKKPGESLKISCKASGYSFTNYWMNWVRQMPGKGLEWMG | 535 |
| W4_VL_VH_ | MIHPSDSEIRLNQKFQGQVTLSVDKSIGTAYMQWSSLKASDTAMYYCSRWY | |
| S56G (CD19 | YLSSPMDYWGQGTTVTVSSASTKGPSVFPLAPSSKSTSGGTAALGCLVKD | |
| Heavy CD3 | YFPEPVTVSWNSGALTSGVHTFPAVLQSSGLYSLSSVVTVPSSSLGTQTYIC | |
| scFv chain) | NVNHKPSNTKVDKRVEPKSCGGGGSGGGGSEIVMTQSPSTLSASVGDRVII | |
| | TCKASQNINNYLNWYQQKPGKAPKWYNTDHLQAGVPSRFSGSGSGAEFT | |
| | LTISSLQPDDFATYYCLQHRSRYTFGQGTKLTVLGGGGGSGGGGSGGGGS | |
| | GGGGSEVQLVETGGGLVQPGGSRRLSCAASGFSLTTYNVHWVRQAPGKG | |
| | LEWVGRMRYSGDTSFNAALTSRFTISRDTSKNTVYLQMNSLRAEDTGVYYC | |
| | ASDPMYIPNYAYGVMNAWGQGTLVTVSSGGGSDKTHTCPPCPAPELLGGP | |
| | SVFLFPPKPKDTLMISRTPEVTCVVVAVSHEDPEVKFNWYVDGVEVHNAKT | |
| | KPREEQYASTYRVVSVLTVLHQDWLNGKEYKCKVSNKALAAPIEKTISKAKG | |
| | QPREPQVCTLPPSREEMTKNQVSLSCAVKGFYPSDIAVEWESNGQPENNY | |
| | KTTPPVLDSDGSFFLVSKLTVDKSRWQQGNVFSCSVMHEALHNHYTQKSL | |
| | SLSPGK | |
| CD3_sp9aF | EVQLVQSGAEVKKPGESLKISCKASGYSFTNYWMNWVRQMPGKGLEWMG | 536 |
| W1_VL_VH | MIHPSDSEIRLNQKFQGQVTLSVDKSIGTAYMQWSSLKASDTAMYYCSRVVY | |
| (CD19 Heavy | YLSSPMDYWGQGTTVTVSSASTKGPSVFPLAPSSKSTSGGTAALGCLVKD | |
| CD3 scFv | YFPEPVTVSWNSGALTSGVHTFPAVLQSSGLYSLSSVVTVPSSSLGTQTYIC | |
| chain) | NVNHKPSNTKVDKRVEPKSCGGGGSGGGGSEIVMTQSPSTLSASVGDRVII | |
| | TCKASQNINNYLNWYQQKPGKAPKWYNTDHLQAGVPSRFSGSGSGAEFT | |
| | LTISSLQPDDFATYYCLQHRSRYTFGQGTKLTVLGGGGGSGGGGSGGGGS | |
| | GGGGSEVQLVETGGGLVQPGGSRRLSCAASGFSLTTYNVHWVRQAPGKG | |
| | LEWVSRMRYSGDTSFNAALTSRFTISRDTSKNTVYLQMNSLRAEDTGVYYC | |
| | ASDPMYIPNYAYGVMNAWGQGTLVTVSSGGGSDKTHTCPPCPAPELLGGP | |
| | SVFLFPPKPKDTLMISRTPEVTCVVVAVSHEDPEVKFNWYVDGVEVHNAKT | |
| | KPREEQYASTYRVVSVLTVLHQDWLNGKEYKCKVSNKALAAPIEKTISKAKG | |
| | QPREPQVCTLPPSREEMTKNQVSLSCAVKGFYPSDIAVEWESNGQPENNY | |
| | KTTPPVLDSDGSFFLVSKLTVDKSRWQQGNVFSCSVMHEALHNHYTQKSL | |
| | SLSPGK | |
| CD3_sp9aF | EVQLVQSGAEVKKPGESLKISCKASGYSFTNYWMNWVRQMPGKGLEWMG | 537 |
| W4_VLVH | MIHPSDSEIRLNQKFQGQVTLSVDKSIGTAYMQWSSLKASDTAMYYCSRWY | |
| (CD19 Heavy | YLSSPMDYWGQGTTVTVSSASTKGPSVFPLAPSSKSTSGGTAALGCLVKD | |
| CD3 scFv | YFPEPVTVSWNSGALTSGVHTFPAVLQSSGLYSLSSVVTVPSSSLGTQTYIC | |
| chain) | NVNHKPSNTKVDKRVEPKSCGGGGSGGGGSEIVMTQSPSTLSASVGDRVII | |
| | TCKASQNINNYLNWYQQKPGKAPKWYNTDHLQAGVPSRFSGSGSGAEFT | |
| | LTISSLQPDDFATYYCLQHRSRYTFGQGTKLTVLGGGGGSGGGGSGGGGS | |
| | GGGGSEVQLVESGGGLVQPGGSLRLSCAASGFSLTTYNVHWVRQAPGKG | |
| | LEWVSRMRYSGDTSFNAALTSRFTISRDNSKNTLYLQMNSLRAEDTAVYYC | |
| | ASDPMYIPNYAYGVMNAWGQGTLVTVSSGGGSDKTHTCPPCPAPELLGGP | |
| | SVFLFPPKPKDTLMISRTPEVTCVVVAVSHEDPEVKFNWYVDGVEVHNAKT | |
| | KPREEQYASTYRVVSVLTVLHQDWLNGKEYKCKVSNKALAAPIEKTISKAKG | |
| | QPREPQVCTLPPSREEMTKNQVSLSCAVKGFYPSDIAVEWESNGQPENNY | |
| | KTTPPVLDSDGSFFLVSKLTVDKSRWQQGNVFSCSVMHEALHNHYTQKSL | |
| | SLSPGK | |
| CD3_sp9ara | EVQLVQSGAEVKKPGESLKISCKASGYSFTNYWMNWVRQMPGKGLEWMG | 538 |
| btor_VHVL | MIHPSDSEIRLNQKFQGQVTLSVDKSIGTAYMQWSSLKASDTAMYYCSRWY | |
| (CD19 Heavy | YLSSPMDYWGQGTTVTVSSASTKGPSVFPLAPSSKSTSGGTAALGCLVKD | |
| CD3 scFv | YFPEPVTVSWNSGALTSGVHTFPAVLQSSGLYSLSSVVTVPSSSLGTQTYIC | |
| chain) | NVNHKPSNTKVDKRVEPKSCGGGGSGGGGSEVQLVESGGGSVQPGGSL | |
| | RLSCTASGFSLTTYNVHWVRQAPGKGLEVVVGRMRYSGDTSFNAALTSRFT | |
| | ISRDTSKNTVYLQMNSLRAEDTATYYCASDPMYIPNYAYGVMNAWGQGTT | |
| | VTVSSGGGGGSGGGGSGGGGSGGGGSEIVMTQSPSTLSASVGDRVIITCK | |
| | ASQNINNYLNWYQQKPGKAPKWYNTDHLQAGVPSRFSGSGSGAEFTLTIS | |
| | SLQPDDFATYYCLQHRSRYTFGQGTKLTVLGGGSDKTHTCPPCPAPELLG | |
| | GPSVFLFPPKPKDTLMISRTPEVTCVVVAVSHEDPEVKFNWYVDGVEVHNA | |
| | KTKPREEQYASTYRVVSVLTVLHQDVVLNGKEYKCKVSNKALAAPIEKTISKA | |
| | KGQPREPQVCTLPPSREEMTKNQVSLSCAVKGFYPSDIAVEVVESNGQPEN | |
| | NYKTTPPVLDSDGSFFLVSKLTVDKSRWQQGNVFSCSVMHEALHNHYTQK | |
| | SLSLSPGK | |
| CD3_sp9ara | EVQLVQSGAEVKKPGESLKISCKASGYSFTNYWMNWVRQMPGKGLEVVMG | 539 |
| btor_VLVH | MIHPSDSEIRLNQKFQGQVTLSVDKSIGTAYMQWSSLKASDTAMYYCSRWY | |
| (CD19 Heavy | YLSSPMDYWGQGTTVTVSSASTKGPSVFPLAPSSKSTSGGTAALGCLVKD | |
| CD3 scFv | YFPEPVTVSWNSGALTSGVHTFPAVLQSSGLYSLSSVVTVPSSSLGTQTYIC | |
| chain) | NVNHKPSNTKVDKRVEPKSCGGGGSGGGGSEIVMTQSPSTLSASVGDRVII | |
| | TCKASQNINNYLNWYQQKPGKAPKWYNTDHLQAGVPSRFSGSGSGAEFT | |
| | LTISSLQPDDFATYYCLQHRSRYTFGQGTKLTVLGGGGGSGGGGSGGGGS | |
| | GGGGSEVQLVESGGGSVQPGGSLRLSCTASGFSLTTYNVHWVRQAPGKG | |
| | LEWVGRMRYSGDTSFNAALTSRFTISRDTSKNTVYLQMNSLRAEDTATYYC | |
| | ASDPMYIPNYAYGVMNAWGQGTTVTVSSGGGSDKTHTCPPCPAPELLGGP | |
| | SVFLFPPKPKDTLMISRTPEVTCVVVAVSHEDPEVKFNWYVDGVEVHNAKT | |
| | KPREEQYASTYRVVSVLTVLHQDWLNGKEYKCKVSNKALAAPIEKTISKAKG | |
| | QPREPQVCTLPPSREEMTKNQVSLSCAVKGFYPSDIAVEWESNGQPENNY | |
| | KTTPPVLDSDGSFFLVSKLTVDKSRWQQGNVFSCSVMHEALHNHYTQKSL | |
| | SLSPGK | |

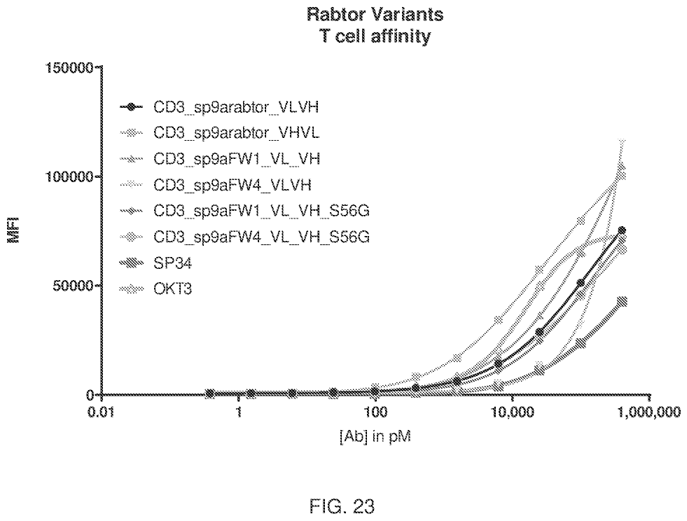

Results of the RTCC assay are shown in FIG. 22. Results of the CD3+ T cell affinity assay are shown in FIG. 23. A summary of the expression and RTCC data for some of the constructs is shown in Table 24.

TABLE 24

| Construct | Expression (mg/L) | RTCC (EC50 in pM) |
|---|---|---|
| CD3_sp9arabtor_VL_VH | 46 | 0.8 |
| CD3_sp9arabtor_VH_VL | 48 | 0.25 |
| CD3_sp9aFW1_VL_VH | 40 | 1.01 |
| sp34 | 21 | 0.39 |

The constructs in the various frameworks were found to have relatively high expression when compared to sp34. CD3_sp9arabtor_VL_VH and CD3_sp9arabtor_VH_VL showed differences in T cell affinity.

### 8.9. Example 9: Anti-CD19-Anti-CD3 Bispecific Antibodies: Sp11a Variants

Bispecific antibodies having a CD3 binding arm based on sp11a (which is the parental antibody of NOV292) in scFv format and a CD19 binding arm in Fab format are produced in the format of FIG. 10, as in Example 7. CDR, VH, and VL sequences for the following constructs of this Example are shown in Table 1:

CD3_sp11a_VHVL_YY_SANSPTM; CD3_sp11a_VHVL_YY_SANSPTM_Y; CD3_sp11a_VHVL_YY_SANSPTM_S; CD3_sp11a_VHVL_YY_Y; CD3_sp11a_VHVL_YY_s; CD3_sp11a_VHVL_SS_SANSPTM; CD3_sp11a_VHVL_SS_SANSPTM_Y; CD3_sp11a_VHVL_SS_SANSPTM_S; CD3_sp11a_VHVL_SS_Y; CD3_sp11a_VHVL_SS_S; CD3_sp11a_VHVL_SS_SANSPTM; CD3_sp11a_VHVL_WS_SANSPTM_Y; CD3_sp11a_VHVL_WS_SANSPTM_S; CD3_sp11a_VHVL_WS_Y; CD3_sp11a_VHVL_WS_S; CD3_sp11a_VHVL_WS_SANSPTM; CD3_sp11a_VHVL_SW_SANSPTM_Y; CD3_sp11a_VHVL_SW_SANSPTM_S; CD3_sp11a_VHVL_SW_Y; CD3_sp11a_VHVL_SW_S; CD3_sp11a_VHVL_SW_SANSPTM; CD3_sp11a_VHVL_TW_SANSPTM_Y; CD3_sp11a_VHVL_TW_SANSPTM_S; CD3_sp11a_VHVL_TW_Y; CD3_sp11a_VHVL_TW_S; CD3_sp11a_VHVL_TW_SANSPTM; CD3_sp11a_VHVL_TT_SANSPTM_Y; CD3_sp11a_VHVL_TT_SANSPTM_S; CD3_sp11a_VHVL_TT_Y; CD3_sp11a_VHVL_TT_S; CD3_sp11a_VHVL_TT_SANSPTM; CD3_SP11AVH3_VLK_3_Y; CD3_SP11AVH3_VLK_3_S; CD3_SP11AVH3_VLK3_Y_PTM; CD3_SP11AVH3_VLK3_S_PTM; CD3_SP11AVH3_VLK_3_Y_SW; CD3_SP11AVH3_VLK_3S_SW; CD3_SP11AVH3_VLK3_Y_PTM_SW; CD3_SP11AVH3_VLK3_S_SWPTM; CD3_SP11AVH3_VLK_SWPTM; CD3_SP11AVH3_VLK_3_SW; CD3_sp11a_VH1_VK2_Y; CD3_sp11a_VH1_VK2_S; CD3_sp11a_VH1_VK2_Y_PTM; CD3_sp11a_VH1_VK2_S_PTM; CD3_sp11a_VH1_VK2_Y_SW; CD3_sp11a_VH1_VK2_S_SW; CD3_sp11a_VH1_VK2_Y_PTM; CD3_sp11a_VH1_VK2_S_PTM_SW; CD3_sp11a_VH1_VK2_SW; CD3_sp11a_VH1_VK2_SW PTM; CD3_SP11A_VH3_VLK1_Y; CD3_SP11A_VH3_VLK1_5; CD3_SP11A_VH3_VLK1_Y_PTM; CD3_SP11A_VH3_VLK1_S_PTM; CD3_SP11A_VH3_VLK1_Y_SW; CD3_SP11A_VH3_VLK1_S_SW; CD3_SP11A_VH3_VLK1_Y_PTM; CD3_SP11A_VH3_VLK1_S_PTM_SW; CD3_SP11A_VH3_VLK1PTM_SW; CD3_SP11A_VH3_VLK1_SW; CD3_SP11A_VH5_VK2_Y; CD3_SP11A_VH5_VK2_5; CD3_SP11A_VH5_VK2_Y_PTM; CD3_SP11A_VH5_VK2_S_PTM; CD3_SP11A_VH5_VK2_Y_SW; CD3_SP11A_VH5_VK2_S_SW; CD3_SP11A_VH5_VK2_Y_PTM_SW; CD3_SP11A_VH5_VK2_S_PTM_SW; CD3_SP11A_VH5_VK2_PTM_SW; CD3_SP11A_VH5_VK2SW.

Full bispecific antibody sequences for several of the constructs are shown in Table 25. CD3 scFv heavy chain polypeptides for the other constructs of this Example are made by replacing the VH and VL sequences in the CD3 scFv heavy chain sequences shown in Table 25 with the respective VH and VL sequences of the other constructs.

TABLE 25

| Construct | | SEQ |
|---|---|---|
| (polypeptide | | ID |
| chain) | Amino Acid Sequence | NO: |
| All | QVQLQESGPGLVKPSETLSLTCTVSGVSLPDYGVSWIRQPPGKGLEWIGVI | 516 |
| Constructs of | WGSETTYYQSSLKSRVTISKDNSKNQVSLKLSSVTAADTAVYYCAKHYYYG | |
| Example | GSYAMDYWGQGTLVTVSSASTKGPSVFPLAPSSKSTSGGTAALGCLVKDY | |
| (CD19 heavy | FPEPVTVSWNSGALTSGVHTFPAVLQSSGLYSLSSVVTVPSSSLGTQTYICN | |
| chain) | VNHKPSNTKVDKRVEPKSCDKTHTCPPCPAPELLGGPSVFLFPPKPKDTLM | |
| | ISRTPEVTCVVVDVSHEDPEVKFNWYVDGVEVHNAKTKPREEQYASTYRVV | |
| | SVLTVLHQDWLNGKEYKCKVSNKALPAPIEKTISKAKGQPREPQVCTLPPSR | |
| | EEMTKNQVSLSCAVKGFYPSDIAVEVVESNGQPENNYKTTPPVLDSDGSFFL | |
| | VSKLTVDKSRWQQGNVFSCSVMHEALHNHYTQKSLSLSPGK | |
| All | EIVMTQSPATLSLSPGERATLSCRASQDISKYLNWYQQKPGQAPRLLIYHTS | 517 |
| Constructs of | RLHSGIPARFSGSGSGTDYTLTISSLQPEDFAVYFCQQGNTLPYTFGQGTKL | |
| Example | EIKRTVAAPSVFIFPPSDEQLKSGTASVVCLLNNFYPREAKVQWKVDNALQS | |
| (CD19 light | GNSQESVTEQDSKDSTYSLSSTLTLSKADYEKHKVYACEVTHQGLSSPVTK | |
| chain) | SFNRGEC | |
| CD3_SP11A | QVQLVESGGGVVQPGRSLRLSCAASGFTFSKNGMHWVRQAPGKGLEWVA | 530 |
| VH3_VLK_3 | MIYYDSSKMYYADTVKGRFTISRDNSKNTLYLQMNSLRAEDTAVYYCASFW | |
| (CD3 scFv | VVDLDFDHWGQGTMVTVSSGGGGSGGGGSGGGGSGGGGSEIVLTQSPGT | |
| heavy chain) | LSLSPGERATLSCRSSQSLVRSEGTTYFNWYQQKPGQAPRLLIYRVSNRFS | |
| | GIPDRFSGSGSGTDFTLTISRLEPEDLAVYYCLQSSHFPWTFGGGTKVEIKG | |
| | GGSDKTHTCPPCPAPELLGGPSVFLFPPKPKDTLMISRTPEVTCVVVDVSH | |
| | EDPEVKFNWYVDGVEVHNAKTKPREEQYASTYRVVSVLTVLHQDWLNGKE | |
| | YKCKVSNKALPAPIEKTISKAKGQPREPQVYTLPPCREEMTKNQVSLW | |
| | CLVKGFYPSDIAVEWESNGQPENNYKTTPPVLDSDGSFFLYSKLTVDKSRW | |
| | QQGNVFSCSVMHEALHNHYTQKSLSLSPGK | |
| CD3_sp11a_ | QVQLVQSGAEVKKPGASVKVSCKASGFTFSKQGMHWVRQAPGQGLEWM | 531 |
| VH1_VK2 | GMIYYDSSKMYYADTVKGRVTMTRDTSTNTLYMELSSLRSEDTAVYYCASF | |
| (CDscFv | VWVDLDFDHWGQGTMVTVSSGGGGSGGGGSGGGGSGGGGSDIVMTQTP | |
| heavy chain) | LSSPVTLGQPASISCRSSQSLVRSDGTTYFNWYQQRPGQPPRLLIYRVSNR | |
| | FSGVPDRFSGSGAGTDFTLKISRVEAEDVGVYYCLQSSHFPVVTFGGGTKV | |
| | EIKGGGSDKTHTCPPCPAPELLGGPSVFLFPPKPKDTLMISRTPEVTCVVVD | |
| | VSHEDPEVKFNWYVDGVEVHNAKTKPREEQYASTYRVVSVLTVLHQDWLN | |
| | GKEYKCKVSNKALPAPIEKTISKAKGQPREPQVYTLPPCREEMTKNQVSLW | |
| | CLVKGFYPSDIAVEWESNGQPENNYKTTPPVLDSDGSFFLYSKLTVDKSRW | |
| | QQGNVFSCSVMHEALHNHYTQKSLSLSPGK | |
| CD3_SP11A | QVQLVESGGGVVQPGRSLRLSCAASGFTFSKNGMHWVRQAPGKGLEWVA | 532 |
| _VH3_VLK1 | MIYYDSSKMYYADTVKGRFTISRDNSKNTLYLQMNSLRAEDTAVYYCASFW | |
| (CD3 scFv | WDLDFDHWGQGTMVTVSSGGGGSGGGGSGGGGSGGGGSDIQMTQSPS | |
| heavy chain) | SLSASVGDRVTITCRSSQSLVRSEGTTYFNWYQQKPGKAPKWYRVSNRF | |
| | SGVPSRFSGSGSGTDFTLTISSLQPEDFATYYCLQSSHFPVVTFGGGTKVEI | |
| | KGGGSDKTHTCPPCPAPELLGGPSVFLFPPKPKDTLMISRTPEVTCVVVDV | |
| | SHEDPEVKFNWYVDGVEVHNAKTKPREEQYASTYRVVSVLTVLHQDWLNG | |
| | KEYKCKVSNKALPAPIEKTISKAKGQPREPQVYTLPPCREEMTKNQVSLWC | |
| | LVKGFYPSDIAVEWESNGQPENNYKTTPPVLDSDGSFFLYSKLTVDKSRWQ | |
| | QGNVFSCSVMHEALHNHYTQKSLSLSPGK | |
| CD3_SP11A | EVQLVQSGAEVKKPGESLKISCKGSGFTFSKQGMHWVRQMPGKGLEWMG | 533 |
| _VH5_VK2 | MIYYDSSKMYYADTVKGQVTISRDNSINTLYLQWSSLKASDTAMYYCASFW | |
| (CD3 scFv | WDLDFDHWGQGTMVTVSSGGGGSGGGGSGGGGSGGGGSDIVMTQTPL | |
| heavy chain) | SSPVTLGQPASISCRSSQSLVRSDGTTYFNWYQQRPGQPPRLLIYRVSNRF | |
| | SGVPDRFSGSGAGTDFTLKISRVEAEDVGVYYCLQSSHFPWTFGGGTKVEI | |
| | KGGGSDKTHTCPPCPAPELLGGPSVFLFPPKPKDTLMISRTPEVTCVVVDV | |
| | SHEDPEVKFNWYVDGVEVHNAKTKPREEQYASTYRVVSVLTVLHQDWLNG | |
| | KEYKCKVSNKALPAPIEKTISKAKGQPREPQVYTLPPCREEMTKNQVSLWC | |
| | LVKGFYPSDIAVEWESNGQPENNYKTTPPVLDSDGSFFLYSKLTVDKSRWQ | |
| | QGNVFSCSVMHEALHNHYTQKSLSLSPGK | |

### 9. SPECIFIC EMBODIMENTS

While various specific embodiments have been illustrated and described, it will be appreciated that various changes can be made without departing from the spirit and scope of the disclosure(s). The present disclosure is exemplified by the numbered embodiments set forth below.

### 9.1. Numbered Embodiments 1 to 1442

1. A CD3 binding molecule that specifically binds to human CD3 and comprises a CDR-H1 sequence, a CDR-H2 sequence a CDR-H3 sequence, a CDR-L1 sequence, a CDR-L2 sequence, and a CDR-L3 sequence set forth in Table 1A, Table 1B, or Table 10.
2. The CD3 binding molecule of embodiment 1, which comprises a CDR-H1 sequence, a CDR-H2 sequence, a CDR-H3 sequence, a CDR-L1 sequence, a CDR-L2 sequence, and a CDR-L3 sequence set forth in Table 1A.
3. The CD3 binding molecule of embodiment 2, wherein the amino acid designated X₁ in Table 1A is T.
4. The CD3 binding molecule of embodiment 2, wherein the amino acid designated X₁ in Table 1A is A.
5. The CD3 binding molecule of any one of embodiments 2 to 4, wherein the amino acid designated X₂ in Table 1A is S.
6. The CD3 binding molecule of any one of embodiments 2 to 4, wherein the amino acid designated X₂ in Table 1A is R.
7. The CD3 binding molecule of any one of embodiments 2 to 6, wherein the amino acid designated X₃ in Table 1A is N.
8. The CD3 binding molecule of any one of embodiments 2 to 6, wherein the amino acid designated X₃ in Table 1A is Y.
9. The CD3 binding molecule of any one of embodiments 2 to 6, wherein the amino acid designated X₃ in Table 1A is Q.
10. The CD3 binding molecule of any one of embodiments 2 to 9, wherein the amino acid designated X₄ in Table 1A is H.
11. The CD3 binding molecule of any one of embodiments 2 to 9, wherein the amino acid designated X₄ in Table 1A is S.
12. The CD3 binding molecule of any one of embodiments 2 to 11, wherein the amino acid designated X₅ in Table 1A is M.
13. The CD3 binding molecule of any one of embodiments 2 to 11, wherein the amino acid designated X₅ in Table 1A is L.
14. The CD3 binding molecule of any one of embodiments 2 to 13, wherein the amino acid designated X₆ in Table 1A is K.
15. The CD3 binding molecule of any one of embodiments 2 to 13, wherein the amino acid designated X₆ in Table 1A is R.
16. The CD3 binding molecule of any one of embodiments 2 to 15, wherein the amino acid designated X₇ in Table 1A is S.
17. The CD3 binding molecule of any one of embodiments 2 to 15, wherein the amino acid designated X₇ in Table 1A is K.
18. The CD3 binding molecule of any one of embodiments 2 to 17, wherein the amino acid designated X₅₅ in Table 1A is F.
19. The CD3 binding molecule of any one of embodiments 2 to 17, wherein the amino acid designated X₅₅ in Table 1A is Y.
20. The CD3 binding molecule of any one of embodiments 2 to 17, wherein the amino acid designated X₅₅ in Table 1A is S.
21. The CD3 binding molecule of any one of embodiments 2 to 20, wherein the amino acid designated X₈ in Table 1A is W.
22. The CD3 binding molecule of any one of embodiments 2 to 20, wherein the amino acid designated X₈ in Table 1A is Y.
23. The CD3 binding molecule of any one of embodiments 2 to 20, wherein the amino acid designated X₈ in Table 1A is S.
24. The CD3 binding molecule of any one of embodiments 2 to 20, wherein the amino acid designated X₈ in Table 1A is T.
25. The CD3 binding molecule of any one of embodiments 2 to 24, wherein the amino acid designated X₉ in Table 1A is W.
26. The CD3 binding molecule of any one of embodiments 2 to 24, wherein the amino acid designated X₉ in Table 1A is Y.
27. The CD3 binding molecule of any one of embodiments 2 to 24, wherein the amino acid designated X₉ in Table 1A is S.
28. The CD3 binding molecule of any one of embodiments 2 to 24, wherein the amino acid designated X₉ in Table 1A is T.
29. The CD3 binding molecule of any one of embodiments 2 to 28, wherein the amino acid designated X₁₀ in Table 1A is H.
30. The CD3 binding molecule of any one of embodiments 2 to 28, wherein the amino acid designated X₁₀ in Table 1A is Y.
31. The CD3 binding molecule of any one of embodiments 2 to 30, wherein the amino acid designated X₁₁ in Table 1A is S.
32. The CD3 binding molecule of any one of embodiments 2 to 30, wherein the amino acid designated X₁₁ in Table 1A is G.
33. The CD3 binding molecule of any one of embodiments 2 to 32, wherein the amino acid designated X₁₂ in Table 1A is I.
34. The CD3 binding molecule of any one of embodiments 2 to 32, wherein the amino acid designated X₁₂ in Table 1A is L.
35. The CD3 binding molecule of any one of embodiments 2 to 34, wherein the amino acid designated X₁₃ in Table 1A is V.
36. The CD3 binding molecule of any one of embodiments 2 to 34, wherein the amino acid designated X₁₃ in Table 1A is G.
37. The CD3 binding molecule of any one of embodiments 2 to 36, wherein the amino acid designated X₁₄ in Table 1A is R.
38. The CD3 binding molecule of any one of embodiments 2 to 36, wherein the amino acid designated X₁₄ in Table 1A is N.
39. The CD3 binding molecule of any one of embodiments 2 to 38, wherein the amino acid designated X₁₅ in Table 1A is D.
40. The CD3 binding molecule of any one of embodiments 2 to 38, wherein the amino acid designated X₁₅ in Table 1A is E.
41. The CD3 binding molecule of any one of embodiments 2 to 38, wherein the amino acid designated X₁₅ in Table 1A is L.
42. The CD3 binding molecule of any one of embodiments 2 to 41, wherein the amino acid designated X₁₆ in Table 1A is G.
43. The CD3 binding molecule of any one of embodiments 2 to 41, wherein the amino acid designated X₁₆ in Table 1A is N.
44. The CD3 binding molecule of any one of embodiments 2 to 41, wherein the amino acid designated X₁₆ in Table 1A is E.
45. The CD3 binding molecule of any one of embodiments 2 to 44, wherein the amino acid designated X₁₇ in Table 1A is R.
46. The CD3 binding molecule of any one of embodiments 2 to 44, wherein the amino acid designated X₁₇ in Table 1A is S.
47. The CD3 binding molecule of any one of embodiments 2 to 46, wherein the amino acid designated X₁₈ in Table 1A is V.
48. The CD3 binding molecule of any one of embodiments 2 to 46, wherein the amino acid designated X₁₈ in Table 1A is T.
49. The CD3 binding molecule of any one of embodiments 2 to 48, wherein the amino acid designated X₁₉ in Table 1A is N.
50. The CD3 binding molecule of any one of embodiments 2 to 48, wherein the amino acid designated X₁₉ in Table 1A is T.
51. The CD3 binding molecule of any one of embodiments 2 to 50, wherein the amino acid designated X₂₀ in Table 1A is R.
52. The CD3 binding molecule of any one of embodiments 2 to 50, wherein the amino acid designated X₂₀ in Table 1A is L.
53. The CD3 binding molecule of any one of embodiments 2 to 52, wherein the amino acid designated X₂₁ in Table 1A is F.
54. The CD3 binding molecule of any one of embodiments 2 to 52, wherein the amino acid designated X₂₁ in Table 1A is E.
55. The CD3 binding molecule of any one of embodiments 2 to 54, wherein the amino acid designated X₂₂ in Table 1A is S.
56. The CD3 binding molecule of any one of embodiments 2 to 54, wherein the amino acid designated X₂₂ in Table 1A is Y.
57. The CD3 binding molecule of any one of embodiments 2 to 56, wherein the amino acid designated X₂₃ in Table 1A is S.
58. The CD3 binding molecule of any one of embodiments 2 to 56, wherein the amino acid designated X₂₃ in Table 1A is Y.
59. The CD3 binding molecule of any one of embodiments 2 to 58, wherein the amino acid designated X₂₄ in Table 1A is S.
60. The CD3 binding molecule of any one of embodiments 2 to 58, wherein the amino acid designated X₂₄ in Table 1A is A.
61. The CD3 binding molecule of any one of embodiments 2 to 60, wherein the amino acid designated X₂₅ in Table 1A is H.
62. The CD3 binding molecule of any one of embodiments 2 to 60, wherein the amino acid designated X₂₅ in Table 1A is T.
63. The CD3 binding molecule of any one of embodiments 2 to 62, wherein the amino acid designated X₂₆ in Table 1A is F.
64. The CD3 binding molecule of any one of embodiments 2 to 62, wherein the amino acid designated X₂₆ in Table 1A is Y.
65. The CD3 binding molecule of any one of embodiments 2 to 64, wherein the amino acid designated X₂₇ in Table 1A is W.
66. The CD3 binding molecule of any one of embodiments 2 to 64, wherein the amino acid designated X₂₇ in Table 1A is Y.
67. The CD3 binding molecule of any one of embodiments 2 to 66, which comprises the CDR-H1 sequence C1-1.
68. The CD3 binding molecule of any one of embodiments 2 to 66, which comprises the CDR-H1 sequence C1-2.
69. The CD3 binding molecule of any one of embodiments 2 to 66, which comprises the CDR-H1 sequence C1-3.
70. The CD3 binding molecule of any one of embodiments 2 to 66, which comprises the CDR-H1 sequence C1-4.
71. The CD3 binding molecule of any one of embodiments 2 to 70, which comprises the CDR-H2 sequence C1-5.
72. The CD3 binding molecule of any one of embodiments 2 to 70, which comprises the CDR-H2 sequence C1-6.
73. The CD3 binding molecule of any one of embodiments 2 to 70, which comprises the CDR-H2 sequence C1-7.
74. The CD3 binding molecule of any one of embodiments 2 to 73, which comprises the CDR-H3 sequence C1-8.
75. The CD3 binding molecule of any one of embodiments 2 to 73, which comprises the CDR-H3 sequence C1-9.
76. The CD3 binding molecule of any one of embodiments 2 to 73, which comprises the CDR-H3 sequence C1-10.
77. The CD3 binding molecule of any one of embodiments 2 to 73, which comprises the CDR-H3 sequence C1-11.
78. The CD3 binding molecule of any one of embodiments 2 to 77, which comprises the CDR-L1 sequence C1-12.
79. The CD3 binding molecule of any one of embodiments 2 to 77, which comprises the CDR-L1 sequence C1-13.
80. The CD3 binding molecule of any one of embodiments 2 to 77, which comprises the CDR-L1 sequence C1-14.
81. The CD3 binding molecule of any one of embodiments 2 to 77, which comprises the CDR-L1 sequence C1-15.
82. The CD3 binding molecule of any one of embodiments 2 to 77, which comprises the CDR-L1 sequence C1-16.
83. The CD3 binding molecule of any one of embodiments 2 to 77, which comprises the CDR-L1 sequence C1-17.
84. The CD3 binding molecule of any one of embodiments 2 to 83, which comprises the CDR-L2 sequence C1-18.
85. The CD3 binding molecule of any one of embodiments 2 to 83, which comprises the CDR-L2 sequence C1-19.
86. The CD3 binding molecule of any one of embodiments 2 to 85, which comprises the CDR-L3 sequence C1-20.
87. The CD3 binding molecule of any one of embodiments 2 to 85, which comprises the CDR-L3 sequence C1-21.
88. The CD3 binding molecule of any one of embodiments 2 to 85, which comprises the CDR-L3 sequence C1-22.
89. The CD3 binding molecule of any one of embodiments 2 to 85, which comprises the CDR-L3 sequence C1-23.
90. The CD3 binding molecule of embodiment 1, which comprises a CDR-H1 sequence, a CDR-H2 sequence, a CDR-H3 sequence, a CDR-L1 sequence, a CDR-L2 sequence, and a CDR-L3 sequence set forth in Table 1B.
91. The CD3 binding molecule of embodiment 90, wherein the amino acid designated X₂₈ in Table 1B is V.
92. The CD3 binding molecule of embodiment 90, wherein the amino acid designated X₂₈ in Table 1B is I.
93. The CD3 binding molecule of any one of embodiments 90 to 92, wherein the amino acid designated X₂₉ in Table 1B is F.
94. The CD3 binding molecule of any one of embodiments 90 to 92, wherein the amino acid designated X₂₉ in Table 1B is Y.
95. The CD3 binding molecule of any one of embodiments 90 to 94, wherein the amino acid designated X₃₀ in Table 1B is N.
96. The CD3 binding molecule of any one of embodiments 90 to 94, wherein the amino acid designated X₃₀ in Table 1B is S.
97. The CD3 binding molecule of any one of embodiments 90 to 96, wherein the amino acid designated X₃₁ in Table 1B is A.
98. The CD3 binding molecule of any one of embodiments 90 to 96, wherein the amino acid designated X₃₁ in Table 1B is S.
99. The CD3 binding molecule of any one of embodiments 90 to 98, wherein the amino acid designated X₃₂ in Table 1B is T.
100. The CD3 binding molecule of any one of embodiments 90 to 98, wherein the amino acid designated X₃₂ in Table 1B is K.
101. The CD3 binding molecule of any one of embodiments 90 to 100, wherein the amino acid designated X₃₃ in Table 1B is T.
102. The CD3 binding molecule of any one of embodiments 90 to 100, wherein the amino acid designated X₃₃ in Table 1B is A.
103. The CD3 binding molecule of any one of embodiments 90 to 102, wherein the amino acid designated X₃₄ in Table 1B is S.
104. The CD3 binding molecule of any one of embodiments 90 to 102, wherein the amino acid designated X₃₄ in Table 1B is R.
105. The CD3 binding molecule of any one of embodiments 90 to 104, wherein the amino acid designated X₃₅ in Table 1B is N.
106. The CD3 binding molecule of any one of embodiments 90 to 104, wherein the amino acid designated X₃₅ in Table 1B is G.
107. The CD3 binding molecule of any one of embodiments 90 to 106, wherein the amino acid designated X₃₆ in Table 1B is S.
108. The CD3 binding molecule of any one of embodiments 90 to 106, wherein the amino acid designated X₃₆ in Table 1B is A.
109. The CD3 binding molecule of any one of embodiments 90 to 108, wherein the amino acid designated X₃₇ in Table 1B is A.
110. The CD3 binding molecule of any one of embodiments 90 to 108, wherein the amino acid designated X₃₇ in Table 1B is T.
111. The CD3 binding molecule of any one of embodiments 90 to 108, wherein the amino acid designated X₃₇ in Table 1B is S.
112. The CD3 binding molecule of any one of embodiments 90 to 111, wherein the amino acid designated X₃₈ in Table 1B is N.
113. The CD3 binding molecule of any one of embodiments 90 to 111, wherein the amino acid designated X₃₈ in Table 1B is D.
114. The CD3 binding molecule of any one of embodiments 90 to 113, wherein the amino acid designated X₃₉ in Table 1B is N.
115. The CD3 binding molecule of any one of embodiments 90 to 113, wherein the amino acid designated X₃₉ in Table 1B is K.
116. The CD3 binding molecule of any one of embodiments 90 to 115, wherein the amino acid designated X₄₀ in Table 1B is D.
117. The CD3 binding molecule of any one of embodiments 90 to 115, wherein the amino acid designated X₄₀ in Table 1B is N.
118. The CD3 binding molecule of any one of embodiments 90 to 117, wherein the amino acid designated X₄₁ in Table 1B is H.
119. The CD3 binding molecule of any one of embodiments 90 to 117, wherein the amino acid designated X₄₁ in Table 1B is N.
120. The CD3 binding molecule of any one of embodiments 90 to 119, wherein the amino acid designated X₄₂ in Table 1B is Q.
121. The CD3 binding molecule of any one of embodiments 90 to 119, wherein the amino acid designated X₄₂ in Table 1B is E.
122. The CD3 binding molecule of any one of embodiments 90 to 121, wherein the amino acid designated X₄₃ in Table 1B is R.
123. The CD3 binding molecule of any one of embodiments 90 to 121, wherein the amino acid designated X₄₃ in Table 1B is S.
124. The CD3 binding molecule of any one of embodiments 90 to 121, wherein the amino acid designated X₄₃ in Table 1B is G.
125. The CD3 binding molecule of any one of embodiments 90 to 124, which comprises the CDR-H1 sequence C2-1.
126. The CD3 binding molecule of any one of embodiments 90 to 124, which comprises the CDR-H1 sequence C2-2.
127. The CD3 binding molecule of any one of embodiments 90 to 124, which comprises the CDR-H1 sequence C2-3.
128. The CD3 binding molecule of any one of embodiments 90 to 124, which comprises the CDR-H1 sequence C2-4.
129. The CD3 binding molecule of any one of embodiments 90 to 128, which comprises the CDR-H2 sequence C2-5.
130. The CD3 binding molecule of any one of embodiments 90 to 128, which comprises the CDR-H2 sequence C2-6.
131. The CD3 binding molecule of any one of embodiments 90 to 128, which comprises the CDR-H2 sequence C2-7.
132. The CD3 binding molecule of any one of embodiments 90 to 131, which comprises the CDR-H3 sequence C2-8.
133. The CD3 binding molecule of any one of embodiments 90 to 131, which comprises the CDR-H3 sequence C2-9.
134. The CD3 binding molecule of any one of embodiments 90 to 133, which comprises the CDR-L1 sequence C2-10.
135. The CD3 binding molecule of any one of embodiments 90 to 133, which comprises the CDR-L1 sequence C2-11.
136. The CD3 binding molecule of any one of embodiments 90 to 133, which comprises the CDR-L1 sequence C2-12.
137. The CD3 binding molecule of any one of embodiments 90 to 136, which comprises the CDR-L2 sequence C2-13.
138. The CD3 binding molecule of any one of embodiments 90 to 136, which comprises the CDR-L2 sequence C2-14.
139. The CD3 binding molecule of any one of embodiments 90 to 136, which comprises the CDR-L2 sequence C2-15.
140. The CD3 binding molecule of any one of embodiments 90 to 139, which comprises the CDR-L3 sequence C2-16.
141. The CD3 binding molecule of any one of embodiments 90 to 139, which comprises the CDR-L3 sequence C2-17.
142. The CD3 binding molecule of embodiment 1, which comprises a CDR-H1 sequence, a CDR-H2 sequence, a CDR-H3 sequence, a CDR-L1 sequence, a CDR-L2 sequence, and a CDR-L3 sequence set forth in Table 10.
143. The CD3 binding molecule of embodiment 142, wherein the amino acid designated X₄₄ in Table 10 is G.
144. The CD3 binding molecule of embodiment 142, wherein the amino acid designated X₄₄ in Table 10 is A.
145. The CD3 binding molecule of any one of embodiments 142 to 144, wherein the amino acid designated X₄₅ in Table 10 is H.
146. The CD3 binding molecule of any one of embodiments 142 to 144, wherein the amino acid designated X₄₅ in Table 10 is N.
147. The CD3 binding molecule of any one of embodiments 142 to 146, wherein the amino acid designated X₄₆ in Table 10 is D.
148. The CD3 binding molecule of any one of embodiments 142 to 146, wherein the amino acid designated X₄₆ in Table 10 is G.
149. The CD3 binding molecule of any one of embodiments 142 to 148, wherein the amino acid designated X₄₇ in Table 10 is A.
150. The CD3 binding molecule of any one of embodiments 142 to 148, wherein the amino acid designated X₄₇ in Table 10 is G.
151. The CD3 binding molecule of any one of embodiments 142 to 150, wherein the amino acid designated X₄₈ in Table 10 is N.
152. The CD3 binding molecule of any one of embodiments 142 to 150, wherein the amino acid designated X₄₈ in Table 10 is K.
153. The CD3 binding molecule of any one of embodiments 142 to 152, wherein the amino acid designated X₄₉ in Table 10 is V.
154. The CD3 binding molecule of any one of embodiments 142 to 152, wherein the amino acid designated X₄₉ in Table 10 is A.
155. The CD3 binding molecule of any one of embodiments 142 to 154, wherein the amino acid designated X₅₀ in Table 10 is N.
156. The CD3 binding molecule of any one of embodiments 142 to 154, wherein the amino acid designated X₅₀ in Table 10 is V.
157. The CD3 binding molecule of any one of embodiments 142 to 156, wherein the amino acid designated X₅₁ in Table 10 is A.
158. The CD3 binding molecule of any one of embodiments 142 to 156, wherein the amino acid designated X₅₁ in Table 10 is V.
159. The CD3 binding molecule of any one of embodiments 142 to 158, wherein the amino acid designated X₅₂ in Table 10 is Y.
160. The CD3 binding molecule of any one of embodiments 142 to 158, wherein the amino acid designated X₅₂ in Table 10 is F.
161. The CD3 binding molecule of any one of embodiments 142 to 160, wherein the amino acid designated X₅₃ in Table 10 is I.
162. The CD3 binding molecule of any one of embodiments 142 to 160, wherein the amino acid designated X₅₃ in Table 10 is V.
163. The CD3 binding molecule of any one of embodiments 142 to 162, wherein the amino acid designated X₅₄ in Table 10 is I.
164. The CD3 binding molecule of any one of embodiments 142 to 162, wherein the amino acid designated X₅₄ in Table 10 is H.
165. The CD3 binding molecule of any one of embodiments 142 to 164, which comprises the CDR-H1 sequence C3-1.
166. The CD3 binding molecule of any one of embodiments 142 to 164, which comprises the CDR-H1 sequence C3-2.
167. The CD3 binding molecule of any one of embodiments 142 to 164, which comprises the CDR-H1 sequence C3-3.
168. The CD3 binding molecule of any one of embodiments 142 to 164, which comprises the CDR-H1 sequence C3-4.
169. The CD3 binding molecule of any one of embodiments 142 to 168, which comprises the CDR-H2 sequence C3-5.
170. The CD3 binding molecule of any one of embodiments 142 to 168, which comprises the CDR-H2 sequence C3-6.
171. The CD3 binding molecule of any one of embodiments 142 to 168, which comprises the CDR-H2 sequence C3-7.
172. The CD3 binding molecule of any one of embodiments 142 to 171, which comprises the CDR-H3 sequence C3-8.
173. The CD3 binding molecule of any one of embodiments 142 to 171, which comprises the CDR-H3 sequence C3-9.
174. The CD3 binding molecule of any one of embodiments 142 to 173, which comprises the CDR-L1 sequence C3-10.
175. The CD3 binding molecule of any one of embodiments 142 to 173, which comprises the CDR-L1 sequence C3-11.
176. The CD3 binding molecule of any one of embodiments 142 to 173, which comprises the CDR-L1 sequence C3-12.
177. The CD3 binding molecule of any one of embodiments 142 to 176, which comprises the CDR-L2 sequence C3-13.
178. The CD3 binding molecule of any one of embodiments 142 to 176, which comprises the CDR-L2 sequence C3-14.
179. The CD3 binding molecule of any one of embodiments 142 to 178, which comprises the CDR-L3 sequence C3-15.
180. The CD3 binding molecule of any one of embodiments 142 to 178, which comprises the CDR-L3 sequence C3-16.
181. A CD3 binding molecule that specifically binds to human CD3 and comprises CDR-H1 CDR-H2, and CDR-H3 sequences set forth in Table 1D-1, Table 1E-1, Table 1F-1, Table 1G-1, Table 1H-1, or Table 1I-1, and the corresponding CDR-L1, CDR-L2, and CDR-L3 sequences set forth in Table 1D-2, Table 1E-2, Table 1F-2, Table 1G-2, Table 1H-2, or Table 1I-2, respectfully.
182. The CD3 binding molecule of embodiment 181, which comprises CDR-H1, CDR-H2, and CDR-H3 sequences set forth in Table 1D-1 and the corresponding CDR-L1, CDR-L2, and CDR-L3 sequences set forth in Table 1D-2.
183. The CD3 binding molecule of embodiment 181, which comprises CDR-H1, CDR-H2, and CDR-H3 sequences set forth in Table 1E-1 and the corresponding CDR-L1, CDR-L2, and CDR-L3 sequences set forth in Table 1E-2.
184. The CD3 binding molecule of embodiment 181, which comprises CDR-H1, CDR-H2, and CDR-H3 sequences set forth in Table 1F-1 and the corresponding CDR-L1, CDR-L2, and CDR-L3 sequences set forth in Table 1F-2.
185. The CD3 binding molecule of embodiment 181, which comprises CDR-H1, CDR-H2, and CDR-H3 sequences set forth in Table 1G-1 and the corresponding CDR-L1, CDR-L2, and CDR-L3 sequences set forth in Table 1G-2.
186. The CD3 binding molecule of embodiment 181, which comprises CDR-H1, CDR-H2, and CDR-H3 sequences set forth in Table 1H-1 and the corresponding CDR-L1, CDR-L2, and CDR-L3 sequences set forth in Table 1H-2.
187. The CD3 binding molecule of embodiment 181, which comprises CDR-H1, CDR-H2, and CDR-H3 sequences set forth in Table 1I-1 and the corresponding CDR-L1, CDR-L2, and CDR-L3 sequences set forth in Table 1I-2.
188. The CD3 binding molecule of any one of embodiments 182 to 187, wherein the CDR-H1, CDR-H2, CDR-H3, CDR-L1, CDR-L2, and CDR-L3 sequences are those of NOV292.
189. The CD3 binding molecule of any one of embodiments 182 to 187, wherein the CDR-H1, CDR-H2, CDR-H3, CDR-L1, CDR-L2, and CDR-L3 sequences are those of NOV123.
190. The CD3 binding molecule of any one of embodiments 182 to 187, wherein the CDR-H1, CDR-H2, CDR-H3, CDR-L1, CDR-L2, and CDR-L3 sequences are those of Sp10b.
191. The CD3 binding molecule of any one of embodiments 182 to 187, wherein the CDR-H1, CDR-H2, CDR-H3, CDR-L1, CDR-L2, and CDR-L3 sequences are those of NOV453.
192. The CD3 binding molecule of any one of embodiments 182 to 187, wherein the CDR-H1, CDR-H2, CDR-H3, CDR-L1, CDR-L2, and CDR-L3 sequences are those of NOV229.
193. The CD3 binding molecule of any one of embodiments 182 to 187, wherein the CDR-H1, CDR-H2, CDR-H3, CDR-L1, CDR-L2, and CDR-L3 sequences are those of NOV110.
194. The CD3 binding molecule of any one of embodiments 182 to 187, wherein the CDR-H1, CDR-H2, CDR-H3, CDR-L1, CDR-L2, and CDR-L3 sequences are those of NOV832.
195. The CD3 binding molecule of any one of embodiments 182 to 187, wherein the CDR-H1, CDR-H2, CDR-H3, CDR-L1, CDR-L2, and CDR-L3 sequences are those of NOV589.
196. The CD3 binding molecule of any one of embodiments 182 to 187, wherein the CDR-H1, CDR-H2, CDR-H3, CDR-L1, CDR-L2, and CDR-L3 sequences are those of NOV580.
197. The CD3 binding molecule of any one of embodiments 182 to 187, wherein the CDR-H1, CDR-H2, CDR-H3, CDR-L1, CDR-L2, and CDR-L3 sequences are those of NOV567.
198. The CD3 binding molecule of any one of embodiments 182 to 187, wherein the CDR-H1, CDR-H2, CDR-H3, CDR-L1, CDR-L2, and CDR-L3 sequences are those of NOV221.
199. The CD3 binding molecule of any one of embodiments 182 to 187, wherein the CDR-H1, CDR-H2, CDR-H3, CDR-L1, CDR-L2, and CDR-L3 sequences are those of CD3_sp11a_bkm1.
200. The CD3 binding molecule of any one of embodiments 182 to 187, wherein the CDR-H1, CDR-H2, CDR-H3, CDR-L1, CDR-L2, and CDR-L3 sequences are those of CD3_SP11a_bkm2.
201. The CD3 binding molecule of any one of embodiments 182 to 187, wherein the CDR-H1, CDR-H2, CDR-H3, CDR-L1, CDR-L2, and CDR-L3 sequences are those of CD3_sp11a_hz0.
202. The CD3 binding molecule of any one of embodiments 182 to 187, wherein the CDR-H1, CDR-H2, CDR-H3, CDR-L1, CDR-L2, and CDR-L3 sequences are those of CD3_SP11A_HZ1.
203. The CD3 binding molecule of any one of embodiments 182 to 187, wherein the CDR-H1, CDR-H2, CDR-H3, CDR-L1, CDR-L2, and CDR-L3 sequences are those of CD3_sp11a_sansPTM_hz1.
204. The CD3 binding molecule of any one of embodiments 182 to 187, wherein the CDR-H1, CDR-H2, CDR-H3, CDR-L1, CDR-L2, and CDR-L3 sequences are those of CD3_sp11a_sansPTM_rat.
205. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_YY.
206. The CD3 binding molecule of any one of embodiments 182 to 187, wherein the CDR-H1, CDR-H2, CDR-H3, CDR-L1, CDR-L2, and CDR-L3 sequences are those of CD3_SP11A_VHVL_SS.
207. The CD3 binding molecule of any one of embodiments 182 to 187, wherein the CDR-H1, CDR-H2, CDR-H3, CDR-L1, CDR-L2, and CDR-L3 sequences are those of CD3_SP11A_VHVL_WS.
208. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SW.
209. The CD3 binding molecule of any one of embodiments 182 to 187, wherein the CDR-H1, CDR-H2, CDR-H3, CDR-L1, CDR-L2, and CDR-L3 sequences are those of CD3_SP11A_VHVL_TT.
210. The CD3 binding molecule of any one of embodiments 182 to 187, wherein the CDR-H1, CDR-H2, CDR-H3, CDR-L1, CDR-L2, and CDR-L3 sequences are those of CD3_SP11A_VHVL_TW.
211. The CD3 binding molecule of any one of embodiments 182 to 187, wherein the CDR-H1, CDR-H2, CDR-H3, CDR-L1, CDR-L2, and CDR-L3 sequences are those of CD3_SP11A_VHVL_WT.
212. The CD3 binding molecule of any one of embodiments 182 to 187, wherein the CDR-H1, CDR-H2, CDR-H3, CDR-L1, CDR-L2, and CDR-L3 sequences are those of CD3_SP11A VH3_VLK_3.
213. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2.
214. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1.
215. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2.
216. The CD3 binding molecule of any one of embodiments 182 to 187, wherein the CDR-H1, CDR-H2, CDR-H3, CDR-L1, CDR-L2, and CDR-L3 sequences are those of CD3_sp9aFW1_VL_VH_S56G.
217. The CD3 binding molecule of any one of embodiments 182 to 187, wherein the CDR-H1, CDR-H2, CDR-H3, CDR-L1, CDR-L2, and CDR-L3 sequences are those of CD3_SP9AFW4_VL_VH_S56G.
218. The CD3 binding molecule of any one of embodiments 182 to 187, wherein the CDR-H1, CDR-H2, CDR-H3, CDR-L1, CDR-L2, and CDR-L3 sequences are those of CD3_sp9aFW1_VL_VH.
219. The CD3 binding molecule of any one of embodiments 182 to 187, wherein the CDR-H1, CDR-H2, CDR-H3, CDR-L1, CDR-L2, and CDR-L3 sequences are those of CD3_sp9aFW4_VLVH.
220. The CD3 binding molecule of any one of embodiments 182 to 187, wherein the CDR-H1, CDR-H2, CDR-H3, CDR-L1, CDR-L2, and CDR-L3 sequences are those of CD3_sp9arabtor_VHVL.
221. The CD3 binding molecule of any one of embodiments 182 to 187, wherein the CDR-H1, CDR-H2, CDR-H3, CDR-L1, CDR-L2, and CDR-L3 sequences are those of CD3_sp9arabtor_VLVH.
222. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_YY_SANSPTM.
223. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_YY_SANSPTM_Y.
224. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_YY_SANSPTM_S.
225. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_YY_Y.
226. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_YY_s.
227. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SS_SANSPTM.
228. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SS_SANSPTM_Y.
229. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SS_SANSPTM_S.
230. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SS_Y.
231. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SS_S.
232. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SS_SANSPTM.
233. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_WS_SANSPTM_Y.
234. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_WS_SANSPTM_S.
235. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_WS_Y.
236. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_WS_S.
237. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_WS_SANSPTM.
238. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SW_SANSPTM_Y.
239. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SW_SANSPTM_S.
240. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SW_Y.
241. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SW_S.
242. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_SW_SANSPTM.
243. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TW_SANSPTM_Y.
244. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TW_SANSPTM_S.
245. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TW_Y.
246. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TW_S.
247. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TW_SANSPTM.
248. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TT_SANSPTM_Y.
249. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TT_SANSPTM_S.
250. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TT_Y.
251. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TT_S.
252. The CD3 binding molecule of any one of embodiments 182 to 187, wherein the CDR-H1, CDR-H2, CDR-H3, CDR-L1, CDR-L2, and CDR-L3 sequences are those of CD3_sp11a_VHVL_TT_SANSPTM.
253. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_3_Y.
254. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_3_S.
255. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_3_Y_PTM.
256. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_3_S_PTM.
257. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_3_Y_SW.
258. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_3_S_SW.
259. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_3_Y_PTM_SW.
260. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_3_S_SWPTM.
261. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_SWPTM.
262. The CD3 binding molecule of any one of embodiments 182 to 187, wherein the CDR-H1, CDR-H2, CDR-H3, CDR-L1, CDR-L2, and CDR-L3 sequences are those of CD3_SP11AVH3_VLK_3_SW.
263. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_Y.
264. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_S.
265. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_Y_PTM.
266. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_S_PTM.
267. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_Y_SW.
268. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_S_SW.
269. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_Y_PTM.
270. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_S_PTM_SW.
271. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_SW.
272. The CD3 binding molecule of any one of embodiments 182 to 187, wherein the CDR-H1, CDR-H2, CDR-H3, CDR-L1, CDR-L2, and CDR-L3 sequences are those of CD3_sp11a_VH1_VK2_SW PTM.
273. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1_Y.
274. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1_S.
275. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1_Y_PTM.
276. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1_S_PTM.
277. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1_Y_SW.
278. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1_S_SW.
279. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1_Y_PTM.
280. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1_S_PTM_SW.
281. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1PTM_SW.
282. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH3_VLK1_SW.
283. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_Y.
284. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_S.
285. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_Y_PTM.
286. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_S_PTM.
287. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_Y_SW.
288. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_S_SW.
289. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_Y_PTM_SW.
290. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_S_PTM_SW.
291. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_PTM_SW.
292. The CD3 binding molecule of any one of embodiments 182 to 187, wherein the CDR-H1, CDR-H2, CDR-H3, CDR-L1, CDR-L2, and CDR-L3 sequences are those of CD3_SP11A_VH5_VK2_SW.
293. A CD3 binding molecule that specifically binds to human CD3 and comprises:

(a) a heavy chain variable region comprising:
(i) a CDR-H1 comprising the amino acid sequence of any one of SEQ ID NOs:133,136, 139 and 142;
(ii) a CDR-H2 comprising the amino acid sequence of any one of SEQ ID NOs:134, 137, 140 and 143; and
(iii) a CDR-H3 comprising the amino acid sequence of any one of SEQ ID NOs:135, 138; 141 and 144;
and

(b) a light chain variable region comprising:
(i) a CDR-L1 comprising the amino acid sequence of any one of SEQ ID NOs:149, 152, 155 and 158;
(ii) a CDR-L2 comprising the amino acid sequence of any one of SEQ ID NOs:150, 153, 156 and 159; and
(iii) a CDR-L3 comprising the amino acid sequence of any one of SEQ ID NOs:151, 154, 157 and 160.
   294. The CD3 binding molecule of embodiment 293, in which the heavy chain variable region comprises a CDR-H1 comprising the amino acid sequence of SEQ ID NO:133, a CDR-H2 comprising the amino acid sequence of SEQ ID NO:134, and a CDR-H3 comprising the amino acid sequence of SEQ ID NO:135, and in which the light chain variable region comprises a CDR-L1 comprising the amino acid sequence of SEQ ID NO:149, a CDR-L2 comprising the amino acid sequence of SEQ ID NO:150, and a CDR-L3 comprising the amino acid sequence of SEQ ID NO:151.
   295. The CD3 binding molecule of embodiment 293, in which the heavy chain variable region comprises a CDR-H1 comprising the amino acid sequence of SEQ ID NO:136, a CDR-H2 comprising the amino acid sequence of SEQ ID NO:137, and a CDR-H3 comprising the amino acid sequence of SEQ ID NO:138, and in which the light chain variable region comprises a CDR-L1 comprising the amino acid sequence of SEQ ID NO:152, a CDR-L2 comprising the amino acid sequence of SEQ ID NO:153, and a CDR-L3 comprising the amino acid sequence of SEQ ID NO:154.
   296. The CD3 binding molecule of embodiment 293, in which the heavy chain variable region comprises a CDR-H1 comprising the amino acid sequence of SEQ ID NO:139, a CDR-H2 comprising the amino acid sequence of SEQ ID NO:140, and a CDR-H3 comprising the amino acid sequence of SEQ ID NO:141, and in which the light chain variable region comprises a CDR-L1 comprising the amino acid sequence of SEQ ID NO:155, a CDR-L2 comprising the amino acid sequence of SEQ ID NO:156, and a CDR-L3 comprising the amino acid sequence of SEQ ID NO:157.
   297. The CD3 binding molecule of embodiment 293, in which the heavy chain variable region comprises a CDR-H1 comprising the amino acid sequence of SEQ ID NO:142, a CDR-H2 comprising the amino acid sequence of SEQ ID NO:143, and a CDR-H3 comprising the amino acid sequence of SEQ ID NO:144, and in which the light chain variable region comprises a CDR-L1 comprising the amino acid sequence of SEQ ID NO:158, a CDR-L2 comprising the amino acid sequence of SEQ ID NO:159, and a CDR-L3 comprising the amino acid sequence of SEQ ID NO:160.
   298. A CD3 binding molecule that specifically binds to human CD3 and comprises:

(a) a heavy chain variable region comprising:
(i) a CDR-H1 comprising the amino acid sequence of any one of SEQ ID NOs:165, 168, 171 and 174;
(ii) a CDR-H2 comprising the amino acid sequence of any one of SEQ ID NOs:166, 169, 172, and 175;
(iii) a CDR-H3 comprising the amino acid sequence of any one of SEQ ID NOs:167, 170, 173 and 176; and

(b) a light chain variable region comprising:
(i) a CDR-L1 comprising the amino acid sequence of any one of SEQ ID NOs:181, 184, 187 and 190;
(ii) a CDR-L2 comprising the amino acid sequence of any one of SEQ ID NOs:182, 185, 188 and 191; and
(iii) a CDR-L3 comprising the amino acid sequence of any one of SEQ ID NOs:183, 186, 189 and 192.
   299. The CD3 binding molecule of embodiment 298, in which the heavy chain variable region comprises a CDR-H1 comprising the amino acid sequence of SEQ ID NO:165, a CDR-H2 comprising the amino acid sequence of SEQ ID NO:166, and a CDR-H3 comprising the amino acid sequence of SEQ ID NO:167, and in which the light chain variable region comprises a CDR-L1 comprising the amino acid sequence of SEQ ID NO:181, a CDR-L2 comprising the amino acid sequence of SEQ ID NO:182, and a CDR-L3 comprising the amino acid sequence of SEQ ID NO:183.
   300. The CD3 binding molecule of embodiment 298, in which the heavy chain variable region comprises a CDR-H1 comprising the amino acid sequence of SEQ ID NO:168, a CDR-H2 comprising the amino acid sequence of SEQ ID NO:169, and a CDR-H3 comprising the amino acid sequence of SEQ ID NO:170, and in which the light chain variable region comprises a CDR-L1 comprising the amino acid sequence of SEQ ID NO:184, a CDR-L2 comprising the amino acid sequence of SEQ ID NO:185, and a CDR-L3 comprising the amino acid sequence of SEQ ID NO:186.
   301. The CD3 binding molecule of embodiment 298, in which the heavy chain variable region comprises a CDR-H1 comprising the amino acid sequence of SEQ ID NO:171, a CDR-H2 comprising the amino acid sequence of SEQ ID NO:172, and a CDR-H3 comprising the amino acid sequence of SEQ ID NO:173, and in which the light chain variable region comprises a CDR-L1 comprising the amino acid sequence of SEQ ID NO:187, a CDR-L2 comprising the amino acid sequence of SEQ ID NO:188, and a CDR-L3 comprising the amino acid sequence of SEQ ID NO:189.
   302. The CD3 binding molecule of embodiment 298, in which the heavy chain variable region comprises a CDR-H1 comprising the amino acid sequence of SEQ ID NO:174, a CDR-H2 comprising the amino acid sequence of SEQ ID NO:175, and a CDR-H3 comprising the amino acid sequence of SEQ ID NO:176, and in which the light chain variable region comprises a CDR-L1 comprising the amino acid sequence of SEQ ID NO:190, a CDR-L2 comprising the amino acid sequence of SEQ ID NO:191, and a CDR-L3 comprising the amino acid sequence of SEQ ID NO:192.
   303. A CD3 binding molecule that specifically binds to human CD3 and comprises:

(a) a heavy chain variable region comprising:
(i) a CDR-H1 comprising the amino acid sequence of any one of SEQ ID NOs:197, 200, 203 and 206;
(ii) a CDR-H2 comprising the amino acid sequence of any one of SEQ ID NOs:198, 201, 204 and 207;
(iii) a CDR-H3 comprising the amino acid sequence of any one of SEQ ID NOs:199, 202, 205 and 208; and

(b) a light chain variable region comprising:
(i) a CDR-L1 comprising the amino acid sequence of any one of SEQ ID NOs:213, 216, 219 and 222,
(ii) a CDR-L2 comprising the amino acid sequence of any one of SEQ ID NOs:214, 217, 220 and 223; and
(iii) a CDR-L3 comprising the amino acid sequence of any one of SEQ ID NOs:215, 218, 221 and 224.
   304. The CD3 binding molecule of embodiment 303, in which the heavy chain variable region comprises a CDR-H1 comprising the amino acid sequence of SEQ ID NO:197, a CDR-H2 comprising the amino acid sequence of SEQ ID NO:198, and a CDR-H3 comprising the amino acid sequence of SEQ ID NO:199, and in which the light chain variable region comprises a CDR-L1 comprising the amino acid sequence of SEQ ID NO:213, a CDR-L2 comprising the amino acid sequence of SEQ ID NO:2214, and a CDR-L3 comprising the amino acid sequence of SEQ ID NO:215.
   305. The CD3 binding molecule of embodiment 303, in which the heavy chain variable region comprises a CDR-H1 comprising the amino acid sequence of SEQ ID NO:200, a CDR-H2 comprising the amino acid sequence of SEQ ID NO:201, and a CDR-H3 comprising the amino acid sequence of SEQ ID NO:202, and in which the light chain variable region comprises a CDR-L1 comprising the amino acid sequence of SEQ ID NO:216, a CDR-L2 comprising the amino acid sequence of SEQ ID NO:217, and a CDR-L3 comprising the amino acid sequence of SEQ ID NO:218.
   306. The CD3 binding molecule of embodiment 303, in which the heavy chain variable region comprises a CDR-H1 comprising the amino acid sequence of SEQ ID NO:203, a CDR-H2 comprising the amino acid sequence of SEQ ID NO:204, and a CDR-H3 comprising the amino acid sequence of SEQ ID NO:205, and in which the light chain variable region comprises a CDR-L1 comprising the amino acid sequence of SEQ ID NO:219, a CDR-L2 comprising the amino acid sequence of SEQ ID NO:220, and a CDR-L3 comprising the amino acid sequence of SEQ ID NO:221.
   307. The CD3 binding molecule of embodiment 303, in which the heavy chain variable region comprises a CDR-H1 comprising the amino acid sequence of SEQ ID NO:206, a CDR-H2 comprising the amino acid sequence of SEQ ID NO:207, and a CDR-H3 comprising the amino acid sequence of SEQ ID NO:208, and in which the light chain variable region comprises a CDR-L1 comprising the amino acid sequence of SEQ ID NO:222, a CDR-L2 comprising the amino acid sequence of SEQ ID NO:223, and a CDR-L3 comprising the amino acid sequence of SEQ ID NO:224.
   308. A CD3 binding molecule that specifically binds to human CD3 and comprises:

(a) a heavy chain variable region comprising:
(i) a CDR-H1 comprising the amino acid sequence of any one of SEQ ID NOs:229, 232, 235 and 238;
(ii) a CDR-H2 comprising the amino acid sequence of any one of SEQ ID NOs:230, 233, 236 and 239, and
(iii) a CDR-H3 comprising the amino acid sequence of any one of SEQ ID NOs:231, 234, 237 and 240;
and

(b) a light chain variable region comprising:
(i) a CDR-L1 comprising the amino acid sequence of any one of SEQ ID NOs:245, 248, 251 and 254;
(ii) a CDR-L2 comprising the amino acid sequence of any one of SEQ ID NOs:246, 249, 252 and 255; and
(iii) a CDR-L3 comprising the amino acid sequence of any one of SEQ ID NOs:247, 250, 253 and 256.
   309. The CD3 binding molecule of embodiment 308, in which the heavy chain variable region comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231, and in which the light chain variable region comprises a CDR-L1 comprising the amino acid sequence of SEQ ID NO:245, a CDR-L2 comprising the amino acid sequence of SEQ ID NO:246, and a CDR-L3 comprising the amino acid sequence of SEQ ID NO:247.
   310. The CD3 binding molecule of embodiment 308, in which the heavy chain variable region comprises a CDR-H1 comprising the amino acid sequence of SEQ ID NO:232, a CDR-H2 comprising the amino acid sequence of SEQ ID NO:233, and a CDR-H3 comprising the amino acid sequence of SEQ ID NO:234, and in which the light chain variable region comprises a CDR-L1 comprising the amino acid sequence of SEQ ID NO:248, a CDR-L2 comprising the amino acid sequence of SEQ ID NO:249, and a CDR-L3 comprising the amino acid sequence of SEQ ID NO:250.
   311. The CD3 binding molecule of embodiment 308, in which the heavy chain variable region comprises a CDR-H1 comprising the amino acid sequence of SEQ ID NO:235, a CDR-H2 comprising the amino acid sequence of SEQ ID NO:236, and a CDR-H3 comprising the amino acid sequence of SEQ ID NO:237, and in which the light chain variable region comprises a CDR-L1 comprising the amino acid sequence of SEQ ID NO:251, a CDR-L2 comprising the amino acid sequence of SEQ ID NO:252, and a CDR-L3 comprising the amino acid sequence of SEQ ID NO:253.
   312. The CD3 binding molecule of embodiment 308, in which the heavy chain variable region comprises a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:240, and in which the light chain variable region comprises a CDR-L1 comprising the amino acid sequence of SEQ ID NO:254, a CDR-L2 comprising the amino acid sequence of SEQ ID NO:255, and a CDR-L3 comprising the amino acid sequence of SEQ ID NO:255.
   313. A CD3 binding molecule that specifically binds to human CD3 and comprises:

(a) a heavy chain variable region comprising:
(i) a CDR-H1 comprising the amino acid sequence of any one of SEQ ID NOs:261, 264, 267 and 270;
(ii) a CDR-H2 comprising the amino acid sequence of any one of SEQ ID NOs:262, 265, 268 and 271; and
(iii) a CDR-H3 comprising the amino acid sequence of any one of SEQ ID NOs:263, 266, 269 and 272;
and

(b) a light chain variable region comprising:
(i) a CDR-L1 comprising the amino acid sequence of any one of SEQ ID NOs:277, 280, 283 and 286;
(ii) a CDR-L2 comprising the amino acid sequence of any one of SEQ ID NOs:278, 281, 284 and 287; and
(iii) a CDR-L3 comprising the amino acid sequence of any one of SEQ ID NOs:279, 282, 285 and 288.
   314. The CD3 binding molecule of embodiment 313, in which the heavy chain variable region comprises a CDR-H1 comprising the amino acid sequence of SEQ ID NO:261, a CDR-H2 comprising the amino acid sequence of SEQ ID NO:262, and a CDR-H3 comprising the amino acid sequence of SEQ ID NO:263, and in which the light chain variable region comprises a CDR-L1 comprising the amino acid sequence of SEQ ID NO:277, a CDR-L2 comprising the amino acid sequence of SEQ ID NO:278, and a CDR-L3 comprising the amino acid sequence of SEQ ID NO:279.
   315. The CD3 binding molecule of embodiment 313, in which the heavy chain variable region comprises a CDR-H1 comprising the amino acid sequence of SEQ ID NO:264, a CDR-H2 comprising the amino acid sequence of SEQ ID NO:265, and a CDR-H3 comprising the amino acid sequence of SEQ ID NO:266, and in which the light chain variable region comprises a CDR-L1 comprising the amino acid sequence of SEQ ID NO:280, a CDR-L2 comprising the amino acid sequence of SEQ ID NO:281, and a CDR-L3 comprising the amino acid sequence of SEQ ID NO:282.
   316. The CD3 binding molecule of embodiment 313, in which the heavy chain variable region comprises a CDR-H1 comprising the amino acid sequence of SEQ ID NO:267, a CDR-H2 comprising the amino acid sequence of SEQ ID NO:268, and a CDR-H3 comprising the amino acid sequence of SEQ ID NO:269, and in which the light chain variable region comprises a CDR-L1 comprising the amino acid sequence of SEQ ID NO:283, a CDR-L2 comprising the amino acid sequence of SEQ ID NO:284, and a CDR-L3 comprising the amino acid sequence of SEQ ID NO:285.
   317. The CD3 binding molecule of embodiment 313, in which the heavy chain variable region comprises a CDR-H1 comprising the amino acid sequence of SEQ ID NO:270, a CDR-H2 comprising the amino acid sequence of SEQ ID NO:271, and a CDR-H3 comprising the amino acid sequence of SEQ ID NO:272, and in which the light chain variable region comprises a CDR-L1 comprising the amino acid sequence of SEQ ID NO:286, a CDR-L2 comprising the amino acid sequence of SEQ ID NO:287, and a CDR-L3 comprising the amino acid sequence of SEQ ID NO:288.
   318. A CD3 binding molecule that specifically binds to human CD3 and comprises:

(a) a heavy chain variable region comprising:
(i) a CDR-H1 comprising the amino acid sequence of any one of SEQ ID NOs:293, 296, 299 and 302;
(ii) a CDR-H2 comprising the amino acid sequence of any one of SEQ ID NOs:294, 297, 300 and 303; and
(iii) a CDR-H3 comprising the amino acid sequence of any one of SEQ ID NOs:295, 298, 301 and 304;
and

(b) a light chain variable region comprising:
(i) a CDR-L1 comprising the amino acid sequence of any one of SEQ ID NOs:309, 312, 315 and 318;
(ii) a CDR-L2 comprising the amino acid sequence of any one of SEQ ID NOs:310, 313, 316 and 319; and
(iii) a CDR-L3 comprising the amino acid sequence of any one of SEQ ID NOs:311, 314, 317 and 320.
   319. The CD3 binding molecule of embodiment 318, in which the heavy chain variable region comprises a CDR-H1 comprising the amino acid sequence of SEQ ID NO:293, a CDR-H2 comprising the amino acid sequence of SEQ ID NO:294, and a CDR-H3 comprising the amino acid sequence of SEQ ID NO:295, and in which the light chain variable region comprises a CDR-L1 comprising the amino acid sequence of SEQ ID NO:309, a CDR-L2 comprising the amino acid sequence of SEQ ID NO:310, and a CDR-L3 comprising the amino acid sequence of SEQ ID NO:311.
   320. The CD3 binding molecule of embodiment 318, in which the heavy chain variable region comprises a CDR-H1 comprising the amino acid sequence of SEQ ID NO:296, a CDR-H2 comprising the amino acid sequence of SEQ ID NO:297, and a CDR-H3 comprising the amino acid sequence of SEQ ID NO:298, and in which the light chain variable region comprises a CDR-L1 comprising the amino acid sequence of SEQ ID NO:312, a CDR-L2 comprising the amino acid sequence of SEQ ID NO:313, and a CDR-L3 comprising the amino acid sequence of SEQ ID NO:314.
   321. The CD3 binding molecule of embodiment 318, in which the heavy chain variable region comprises a CDR-H1 comprising the amino acid sequence of SEQ ID NO:299, a CDR-H2 comprising the amino acid sequence of SEQ ID NO:300, and a CDR-H3 comprising the amino acid sequence of SEQ ID NO:301, and in which the light chain variable region comprises a CDR-L1 comprising the amino acid sequence of SEQ ID NO:315, a CDR-L2 comprising the amino acid sequence of SEQ ID NO:316, and a CDR-L3 comprising the amino acid sequence of SEQ ID NO:317.
   322. The CD3 binding molecule of embodiment 318, in which the heavy chain variable region comprises a CDR-H1 comprising the amino acid sequence of SEQ ID NO:302, a CDR-H2 comprising the amino acid sequence of SEQ ID NO:303, and a CDR-H3 comprising the amino acid sequence of SEQ ID NO:304, and in which the light chain variable region comprises a CDR-L1 comprising the amino acid sequence of SEQ ID NO:318, a CDR-L2 comprising the amino acid sequence of SEQ ID NO:319, and a CDR-L3 comprising the amino acid sequence of SEQ ID NO:320.
   323. A CD3 binding molecule that specifically binds to human CD3 and comprises:

(a) a heavy chain variable region comprising:
(i) a CDR-H1 comprising the amino acid sequence of any one of SEQ ID NOs:325, 328, 331 and 334;
(ii) a CDR-H2 comprising the amino acid sequence of any one of SEQ ID NOs:326, 329, 332 and 335; and
(iii) a CDR-H3 comprising the amino acid sequence of any one of SEQ ID NOs:327, 330, 333 and 336;
and

(b) a light chain variable region comprising:
(i) a CDR-L1 comprising the amino acid sequence of any one of SEQ ID NOs:341, 344, 347 and 350;
(ii) a CDR-L2 comprising the amino acid sequence of any one of SEQ ID NOs:342, 345, 348 and 351; and
(iii) a CDR-L3 comprising the amino acid sequence of any one of SEQ ID NOs:343, 346, 349 and 352.
   324. The CD3 binding molecule of embodiment 323, in which the heavy chain variable region comprises a CDR-H1 comprising the amino acid sequence of SEQ ID NO:325, a CDR-H2 comprising the amino acid sequence of SEQ ID NO:326, and a CDR-H3 comprising the amino acid sequence of SEQ ID NO:327, and in which the light chain variable region comprises a CDR-L1 comprising the amino acid sequence of SEQ ID NO:341, a CDR-L2 comprising the amino acid sequence of SEQ ID NO:342, and a CDR-L3 comprising the amino acid sequence of SEQ ID NO:343.
   325. The CD3 binding molecule of embodiment 323, in which the heavy chain variable region comprises a CDR-H1 comprising the amino acid sequence of SEQ ID NO:328, a CDR-H2 comprising the amino acid sequence of SEQ ID NO:329, and a CDR-H3 comprising the amino acid sequence of SEQ ID NO:330, and in which the light chain variable region comprises a CDR-L1 comprising the amino acid sequence of SEQ ID NO:344, a CDR-L2 comprising the amino acid sequence of SEQ ID NO:345, and a CDR-L3 comprising the amino acid sequence of SEQ ID NO:346.
   326. The CD3 binding molecule of embodiment 323, in which the heavy chain variable region comprises a CDR-H1 comprising the amino acid sequence of SEQ ID NO:331, a CDR-H2 comprising the amino acid sequence of SEQ ID NO:332, and a CDR-H3 comprising the amino acid sequence of SEQ ID NO:333, and in which the light chain variable region comprises a CDR-L1 comprising the amino acid sequence of SEQ ID NO:347, a CDR-L2 comprising the amino acid sequence of SEQ ID NO:348, and a CDR-L3 comprising the amino acid sequence of SEQ ID NO:349.
   327. The CD3 binding molecule of embodiment 323, in which the heavy chain variable region comprises a CDR-H1 comprising the amino acid sequence of SEQ ID NO:334, a CDR-H2 comprising the amino acid sequence of SEQ ID NO:335, and a CDR-H3 comprising the amino acid sequence of SEQ ID NO:336, and in which the light chain variable region comprises a CDR-L1 comprising the amino acid sequence of SEQ ID NO:350, a CDR-L2 comprising the amino acid sequence of SEQ ID NO:351, and a CDR-L3 comprising the amino acid sequence of SEQ ID NO:352.
   328. A CD3 binding molecule that specifically binds to human CD3 and comprises:

(a) a heavy chain variable region comprising:
(i) a CDR-H1 comprising the amino acid sequence of any one of SEQ ID NOs:357, 360, 363 and 366;
(ii) a CDR-H2 comprising the amino acid sequence of any one of SEQ ID NOs:358, 361, 364 and 367; and
(iii) a CDR-H3 comprising the amino acid sequence of any one of SEQ ID NOs:359, 362, 365 and 368;
and

(b) a light chain variable region comprising:
(i) a CDR-L1 comprising the amino acid sequence of any one of SEQ ID NOs:373, 376, 379 and 382;
(ii) a CDR-L2 comprising the amino acid sequence of any one of SEQ ID NOs:374, 377, 380 and 383; and
(iii) a CDR-L3 comprising the amino acid sequence of any one of SEQ ID NOs:375, 378, 381 and 384.
   329. The CD3 binding molecule of embodiment 328, in which the heavy chain variable region comprises a CDR-H1 comprising the amino acid sequence of SEQ ID NO:357, a CDR-H2 comprising the amino acid sequence of SEQ ID NO:358, and a CDR-H3 comprising the amino acid sequence of SEQ ID NO:359, and in which the light chain variable region comprises a CDR-L1 comprising the amino acid sequence of SEQ ID NO:373, a CDR-L2 comprising the amino acid sequence of SEQ ID NO:374, and a CDR-L3 comprising the amino acid sequence of SEQ ID NO:375.
   330. The CD3 binding molecule of embodiment 328, in which the heavy chain variable region comprises a CDR-H1 comprising the amino acid sequence of SEQ ID NO:360, a CDR-H2 comprising the amino acid sequence of SEQ ID NO:361, and a CDR-H3 comprising the amino acid sequence of SEQ ID NO:362, and in which the light chain variable region comprises a CDR-L1 comprising the amino acid sequence of SEQ ID NO:376, a CDR-L2 comprising the amino acid sequence of SEQ ID NO:377, and a CDR-L3 comprising the amino acid sequence of SEQ ID NO:378.
   331. The CD3 binding molecule of embodiment 328, in which the heavy chain variable region comprises a CDR-H1 comprising the amino acid sequence of SEQ ID NO:363, a CDR-H2 comprising the amino acid sequence of SEQ ID NO:364, and a CDR-H3 comprising the amino acid sequence of SEQ ID NO:365, and in which the light chain variable region comprises a CDR-L1 comprising the amino acid sequence of SEQ ID NO:379, a CDR-L2 comprising the amino acid sequence of SEQ ID NO:380, and a CDR-L3 comprising the amino acid sequence of SEQ ID NO:381.
   332. The CD3 binding molecule of embodiment 328, in which the heavy chain variable region comprises a CDR-H1 comprising the amino acid sequence of SEQ ID NO:366, a CDR-H2 comprising the amino acid sequence of SEQ ID NO:367, and a CDR-H3 comprising the amino acid sequence of SEQ ID NO:368, and in which the light chain variable region comprises a CDR-L1 comprising the amino acid sequence of SEQ ID NO:382, a CDR-L2 comprising the amino acid sequence of SEQ ID NO:383, and a CDR-L3 comprising the amino acid sequence of SEQ ID NO:384.
   333. A CD3 binding molecule that specifically binds to human CD3 and comprises:

(a) a heavy chain variable region comprising:
(i) a CDR-H1 comprising the amino acid sequence of any one of SEQ ID NOs:389, 392, 395 and 398;
(ii) a CDR-H2 comprising the amino acid sequence of any one of SEQ ID NOs:390, 393, 396 and 399; and
(iii) a CDR-H3 comprising the amino acid sequence of any one of SEQ ID NOs:391, 394, 397 and 400;
and

(b) a light chain variable region comprising:
(i) a CDR-L1 comprising the amino acid sequence of any one of SEQ ID NOs:405, 408, 411 and 414;
(ii) a CDR-L2 comprising the amino acid sequence of any one of SEQ ID NOs:406, 409, 412 and 415; and
(iii) a CDR-L3 comprising the amino acid sequence of any one of SEQ ID NOs:407, 410, 413 and 416.
   334. The CD3 binding molecule of embodiment 333, in which the heavy chain variable region comprises a CDR-H1 comprising the amino acid sequence of SEQ ID NO:389, a CDR-H2 comprising the amino acid sequence of SEQ ID NO:390, and a CDR-H3 comprising the amino acid sequence of SEQ ID NO:391, and in which the light chain variable region comprises a CDR-L1 comprising the amino acid sequence of SEQ ID NO:405, a CDR-L2 comprising the amino acid sequence of SEQ ID NO:406, and a CDR-L3 comprising the amino acid sequence of SEQ ID NO:407.
   335. The CD3 binding molecule of embodiment 333, in which the heavy chain variable region comprises a CDR-H1 comprising the amino acid sequence of SEQ ID NO:392, a CDR-H2 comprising the amino acid sequence of SEQ ID NO:393, and a CDR-H3 comprising the amino acid sequence of SEQ ID NO:394, and in which the light chain variable region comprises a CDR-L1 comprising the amino acid sequence of SEQ ID NO:408, a CDR-L2 comprising the amino acid sequence of SEQ ID NO:409, and a CDR-L3 comprising the amino acid sequence of SEQ ID NO:410.
   336. The CD3 binding molecule of embodiment 333, in which the heavy chain variable region comprises a CDR-H1 comprising the amino acid sequence of SEQ ID NO:395, a CDR-H2 comprising the amino acid sequence of SEQ ID NO:396, and a CDR-H3 comprising the amino acid sequence of SEQ ID NO:397, and in which the light chain variable region comprises a CDR-L1 comprising the amino acid sequence of SEQ ID NO:411, a CDR-L2 comprising the amino acid sequence of SEQ ID NO:412, and a CDR-L3 comprising the amino acid sequence of SEQ ID NO:413.
   337. The CD3 binding molecule of embodiment 333, in which the heavy chain variable region comprises a CDR-H1 comprising the amino acid sequence of SEQ ID NO:398, a CDR-H2 comprising the amino acid sequence of SEQ ID NO:399, and a CDR-H3 comprising the amino acid sequence of SEQ ID NO:400, and in which the light chain variable region comprises a CDR-L1 comprising the amino acid sequence of SEQ ID NO:414, a CDR-L2 comprising the amino acid sequence of SEQ ID NO:415, and a CDR-L3 comprising the amino acid sequence of SEQ ID NO:416.
   338. A CD3 binding molecule that specifically binds to human CD3 and comprises:

(a) a heavy chain variable region comprising:
(i) a CDR-H1 comprising the amino acid sequence of any one of SEQ ID NOs:421, 424, 427 and 430;
(ii) a CDR-H2 comprising the amino acid sequence of any one of SEQ ID NOs:422, 425, 428 and 431; and
(iii) a CDR-H3 comprising the amino acid sequence of any one of SEQ ID NOs:423, 426, 429 and 432;
and

(b) a light chain variable region that comprises:
(i) a CDR-L1 comprising the amino acid sequence of any one of SEQ ID NOs:437, 440, 443 and 446;
(ii) a CDR-L2 comprising the amino acid sequence of any one of SEQ ID NOs:438, 441, 444 and 447; and
(iii) a CDR-L3 comprising the amino acid sequence of any one of SEQ ID NOs:439, 442, 445 and 448.
   339. The CD3 binding molecule of embodiment 338, in which the heavy chain variable region comprises a CDR-H1 comprising the amino acid sequence of SEQ ID NO:421, a CDR-H2 comprising the amino acid sequence of SEQ ID NO:422, and a CDR-H3 comprising the amino acid sequence of SEQ ID NO:423, and in which the light chain variable region comprises a CDR-L1 comprising the amino acid sequence of SEQ ID NO:437, a CDR-L2 comprising the amino acid sequence of SEQ ID NO:438, and a CDR-L3 comprising the amino acid sequence of SEQ ID NO:439.
   340. The CD3 binding molecule of embodiment 338, in which the heavy chain variable region comprises a CDR-H1 comprising the amino acid sequence of SEQ ID NO:424, a CDR-H2 comprising the amino acid sequence of SEQ ID NO:425, and a CDR-H3 comprising the amino acid sequence of SEQ ID NO:426, and in which the light chain variable region comprises a CDR-L1 comprising the amino acid sequence of SEQ ID NO:440, a CDR-L2 comprising the amino acid sequence of SEQ ID NO:441, and a CDR-L3 comprising the amino acid sequence of SEQ ID NO:442.
   341. The CD3 binding molecule of embodiment 338, in which the heavy chain variable region comprises a CDR-H1 comprising the amino acid sequence of SEQ ID NO:427, a CDR-H2 comprising the amino acid sequence of SEQ ID NO:428, and a CDR-H3 comprising the amino acid sequence of SEQ ID NO:429, and in which the light chain variable region comprises a CDR-L1 comprising the amino acid sequence of SEQ ID NO:443, a CDR-L2 comprising the amino acid sequence of SEQ ID NO:444, and a CDR-L3 comprising the amino acid sequence of SEQ ID NO:445.
   342. The CD3 binding molecule of embodiment 338, in which the heavy chain variable region comprises a CDR-H1 comprising the amino acid sequence of SEQ ID NO:430, a CDR-H2 comprising the amino acid sequence of SEQ ID NO:431, and a CDR-H3 comprising the amino acid sequence of SEQ ID NO:432, and in which the light chain variable region comprises a CDR-L1 comprising the amino acid sequence of SEQ ID NO:446, a CDR-L2 comprising the amino acid sequence of SEQ ID NO:447, and a CDR-L3 comprising the amino acid sequence of SEQ ID NO:448.
   343. The CD3 binding molecule of embodiment 181, which comprises a heavy chain variable sequence set forth in Table 1J-1 and the corresponding light chain variable sequence set forth in Table 1J-2.
   344. The CD3 binding molecule of embodiment 343, wherein the heavy chain variable sequence and the corresponding light chain variable sequence are those of NOV292.
   345. The CD3 binding molecule of embodiment 343, wherein the heavy chain variable sequence and the corresponding light chain variable sequence are those of NOV123.
   346. The CD3 binding molecule of embodiment 343, wherein the heavy chain variable sequence and the corresponding light chain variable sequence are those of Sp10b.
   347. The CD3 binding molecule of embodiment 343, wherein the heavy chain variable sequence and the corresponding light chain variable sequence are those of NOV453.
   348. The CD3 binding molecule of embodiment 343, wherein the heavy chain variable sequence and the corresponding light chain variable sequence are those of NOV229.
   349. The CD3 binding molecule of embodiment 343, wherein the heavy chain variable sequence and the corresponding light chain variable sequence are those of NOV110.
   350. The CD3 binding molecule of embodiment 343, wherein the heavy chain variable sequence and the corresponding light chain variable sequence are those of NOV832.
   351. The CD3 binding molecule of embodiment 343, wherein the heavy chain variable sequence and the corresponding light chain variable sequence are those of NOV589.
   352. The CD3 binding molecule of embodiment 343, wherein the heavy chain variable sequence and the corresponding light chain variable sequence are those of NOV580.
   353. The CD3 binding molecule of embodiment 343, wherein the heavy chain variable sequence and the corresponding light chain variable sequence are those of NOV567.
   354. The CD3 binding molecule of embodiment 343, wherein the heavy chain variable sequence and the corresponding light chain variable sequence are those of NOV221.
   355. The CD3 binding molecule of embodiment 343, wherein the heavy chain variable sequence and the corresponding light chain variable sequence are those of CD3_sp11a_bkm1.
   356. The CD3 binding molecule of embodiment 343, wherein the heavy chain variable sequence and the corresponding light chain variable sequence are those of CD3_SP11a_bkm2.
   357. The CD3 binding molecule of embodiment 343, wherein the heavy chain variable sequence and the corresponding light chain variable sequence are those of CD3_sp11a_hz0.
   358. The CD3 binding molecule of embodiment 343, wherein the heavy chain variable sequence and the corresponding light chain variable sequence are those of CD3_SP11A_HZ1.
   359. The CD3 binding molecule of embodiment 343, wherein the heavy chain variable sequence and the corresponding light chain variable sequence are those of CD3_sp11a_sansPTM_hz1.
   360. The CD3 binding molecule of embodiment 343, wherein the heavy chain variable sequence and the corresponding light chain variable sequence are those of CD3_sp11a_sansPTM_rat.
   361. The CD3 binding molecule of embodiment 343, wherein the heavy chain variable sequence and the corresponding light chain variable sequence are those of CD3_sp11a_VHVL_YY.
   362. The CD3 binding molecule of embodiment 343, wherein the heavy chain variable sequence and the corresponding light chain variable sequence are those of CD3_SP11A_VHVL_SS.
   363. The CD3 binding molecule of embodiment 343, wherein the heavy chain variable sequence and the corresponding light chain variable sequence are those of CD3_SP11A_VHVL_WS.
   364. The CD3 binding molecule of embodiment 343, wherein the heavy chain variable sequence and the corresponding light chain variable sequence are those of CD3_sp11a_VHVL_SW.
   365. The CD3 binding molecule of embodiment 343, wherein the heavy chain variable sequence and the corresponding light chain variable sequence are those of CD3_SP11A_VHVL_TT.
   366. The CD3 binding molecule of embodiment 343, wherein the heavy chain variable sequence and the corresponding light chain variable sequence are those of CD3_SP11A_VHVL_TW.
   367. The CD3 binding molecule of embodiment 343, wherein the heavy chain variable sequence and the corresponding light chain variable sequence are those of CD3_SP11A_VHVL_WT.
   368. The CD3 binding molecule of embodiment 343, wherein the heavy chain variable sequence and the corresponding light chain variable sequence are those of CD3_SP11A VH3_VLK_3.
   369. The CD3 binding molecule of embodiment 343, wherein the heavy chain variable sequence and the corresponding light chain variable sequence are those of CD3_sp11a_VH1_VK2.
   370. The CD3 binding molecule of embodiment 343, wherein the heavy chain variable sequence and the corresponding light chain variable sequence are those of CD3_SP11A_VH3_VLK1.
   371. The CD3 binding molecule of embodiment 343, wherein the heavy chain variable sequence and the corresponding light chain variable sequence are those of CD3_SP11A_VH5_VK2.
   372. The CD3 binding molecule of embodiment 343, wherein the heavy chain variable sequence and the corresponding light chain variable sequence are those of CD3_sp9aFW1_VL_VH_S56G.
   373. The CD3 binding molecule of embodiment 343, wherein the heavy chain variable sequence and the corresponding light chain variable sequence are those of CD3_SP9AFW4_VL_VH_S56G.
   374. The CD3 binding molecule of embodiment 343, wherein the heavy chain variable sequence and the corresponding light chain variable sequence are those of CD3_sp9aFW1_VL_VH.
   375. The CD3 binding molecule of embodiment 343, wherein the heavy chain variable sequence and the corresponding light chain variable sequence are those of CD3_sp9aFW4_VLVH.
   376. The CD3 binding molecule of embodiment 343, wherein the heavy chain variable sequence and the corresponding light chain variable sequence are those of CD3_sp9arabtor_VHVL.
   377. The CD3 binding molecule of embodiment 343, wherein the heavy chain variable sequence and the corresponding light chain variable sequence are those of CD3_sp9arabtor_VLVH.
   378. The CD3 binding molecule of embodiment 343, wherein the heavy chain variable sequence and the corresponding light chain variable sequence are those of CD3_sp11a_VHVL_YY_SANSPTM.
   379. The CD3 binding molecule of embodiment 343, wherein the heavy chain variable sequence and the corresponding light chain variable sequence are those of CD3_sp11a_VHVL_YY_SANSPTM_Y.
   380. The CD3 binding molecule of embodiment 343, wherein the heavy chain variable sequence and the corresponding light chain variable sequence are those of CD3_sp11a_VHVL_YY_SANSPTM_S.
   381. The CD3 binding molecule of embodiment 343, wherein the heavy chain variable sequence and the corresponding light chain variable sequence are those of CD3_sp11a_VHVL_YY_Y.
   382. The CD3 binding molecule of embodiment 343, wherein the heavy chain variable sequence and the corresponding light chain variable sequence are those of CD3_sp11a_VHVL_YY_s.
   383. The CD3 binding molecule of embodiment 343, wherein the heavy chain variable sequence and the corresponding light chain variable sequence are those of CD3_sp11a_VHVL_SS_SANSPTM.
   384. The CD3 binding molecule of embodiment 343, wherein the heavy chain variable sequence and the corresponding light chain variable sequence are those of CD3_sp11a_VHVL_SS_SANSPTM_Y.
   385. The CD3 binding molecule of embodiment 343, wherein the heavy chain variable sequence and the corresponding light chain variable sequence are those of CD3_sp11a_VHVL_SS_SANSPTM_S.
   386. The CD3 binding molecule of embodiment 343, wherein the heavy chain variable sequence and the corresponding light chain variable sequence are those of CD3_sp11a_VHVL_SS_Y.
   387. The CD3 binding molecule of embodiment 343, wherein the heavy chain variable sequence and the corresponding light chain variable sequence are those of CD3_sp11a_VHVL_SS_S.
   388. The CD3 binding molecule of embodiment 343, wherein the heavy chain variable sequence and the corresponding light chain variable sequence are those of CD3_sp11a_VHVL_SS_SANSPTM.
   389. The CD3 binding molecule of embodiment 343, wherein the heavy chain variable sequence and the corresponding light chain variable sequence are those of CD3_sp11a_VHVL_WS_SANSPTM_Y.
   390. The CD3 binding molecule of embodiment 343, wherein the heavy chain variable sequence and the corresponding light chain variable sequence are those of CD3_sp11a_VHVL_S_SANSPTM_S.
   391. The CD3 binding molecule of embodiment 343, wherein the heavy chain variable sequence and the corresponding light chain variable sequence are those of CD3_sp11a_VHVL_WS_Y.
   392. The CD3 binding molecule of embodiment 343, wherein the heavy chain variable sequence and the corresponding light chain variable sequence are those of CD3_sp11a_VHVL_WS_S.
   393. The CD3 binding molecule of embodiment 343, wherein the heavy chain variable sequence and the corresponding light chain variable sequence are those of CD3_sp11a_VHVL_WS_SANSPTM.
   394. The CD3 binding molecule of embodiment 343, wherein the heavy chain variable sequence and the corresponding light chain variable sequence are those of CD3_sp11a_VHVL_SW_SANSPTM_Y.
   395. The CD3 binding molecule of embodiment 343, wherein the heavy chain variable sequence and the corresponding light chain variable sequence are those of CD3_sp11a_VHVL_SW_SANSPTM_S.
   396. The CD3 binding molecule of embodiment 343, wherein the heavy chain variable sequence and the corresponding light chain variable sequence are those of CD3_sp11a_VHVL_SW_Y.
   397. The CD3 binding molecule of embodiment 343, wherein the heavy chain variable sequence and the corresponding light chain variable sequence are those of CD3_sp11a_VHVL_SW_S.
   398. The CD3 binding molecule of embodiment 343, wherein the heavy chain variable sequence and the corresponding light chain variable sequence are those of CD3_sp11a_VHVL_SW_SANSPTM.
   399. The CD3 binding molecule of embodiment 343, wherein the heavy chain variable sequence and the corresponding light chain variable sequence are those of CD3_sp11a_VHVL_TW_SANSPTM_Y.
   400. The CD3 binding molecule of embodiment 343, wherein the heavy chain variable sequence and the corresponding light chain variable sequence are those of CD3_sp11a_VHVL_TW_SANSPTM_S.
   401. The CD3 binding molecule of embodiment 343, wherein the heavy chain variable sequence and the corresponding light chain variable sequence are those of CD3_sp11a_VHVL_TW_Y.
   402. The CD3 binding molecule of embodiment 343, wherein the heavy chain variable sequence and the corresponding light chain variable sequence are those of CD3_sp11a_VHVL_TW_S.
   403. The CD3 binding molecule of embodiment 343, wherein the heavy chain variable sequence and the corresponding light chain variable sequence are those of CD3_sp11a_VHVL_TW_SANSPTM.
   404. The CD3 binding molecule of embodiment 343, wherein the heavy chain variable sequence and the corresponding light chain variable sequence are those of CD3_sp11a_VHVL_TT_SANSPTM_Y.
   405. The CD3 binding molecule of embodiment 343, wherein the heavy chain variable sequence and the corresponding light chain variable sequence are those of CD3_sp11a_VHVL_TT_SANSPTM_S.
   406. The CD3 binding molecule of embodiment 343, wherein the heavy chain variable sequence and the corresponding light chain variable sequence are those of CD3_sp11a_VHVL_TT_Y.
   407. The CD3 binding molecule of embodiment 343, wherein the heavy chain variable sequence and the corresponding light chain variable sequence are those of CD3_sp11a_VHVL_TT_S.
   408. The CD3 binding molecule of embodiment 343, wherein the heavy chain variable sequence and the corresponding light chain variable sequence are those of CD3_sp11a_VHVL_TT_SANSPTM.
   409. The CD3 binding molecule of embodiment 343, wherein the heavy chain variable sequence and the corresponding light chain variable sequence are those of CD3_SP11AVH3_VLK_3_Y.
   410. The CD3 binding molecule of embodiment 343, wherein the heavy chain variable sequence and the corresponding light chain variable sequence are those of CD3_SP11AVH3_VLK_3_S.
   411. The CD3 binding molecule of embodiment 343, wherein the heavy chain variable sequence and the corresponding light chain variable sequence are those of CD3_SP11AVH3_VLK_3_Y_PTM.
   412. The CD3 binding molecule of embodiment 343, wherein the heavy chain variable sequence and the corresponding light chain variable sequence are those of CD3_SP11AVH3_VLK_3_S_PTM.
   413. The CD3 binding molecule of embodiment 343, wherein the heavy chain variable sequence and the corresponding light chain variable sequence are those of CD3_SP11AVH3_VLK_3_Y_SW.
   414. The CD3 binding molecule of embodiment 343, wherein the heavy chain variable sequence and the corresponding light chain variable sequence are those of CD3_SP11AVH3_VLK_3_S_SW.
   415. The CD3 binding molecule of embodiment 343, wherein the heavy chain variable sequence and the corresponding light chain variable sequence are those of CD3_SP11AVH3_VLK_3_Y_PTM_SW.
   416. The CD3 binding molecule of embodiment 343, wherein the heavy chain variable sequence and the corresponding light chain variable sequence are those of CD3_SP11AVH3_VLK_3_S_SWPTM.
   417. The CD3 binding molecule of embodiment 343, wherein the heavy chain variable sequence and the corresponding light chain variable sequence are those of CD3_SP11AVH3_VLK_SWPTM.
   418. The CD3 binding molecule of embodiment 343, wherein the heavy chain variable sequence and the corresponding light chain variable sequence are those of CD3_SP11AVH3_VLK_3_SW.
   419. The CD3 binding molecule of embodiment 343, wherein the heavy chain variable sequence and the corresponding light chain variable sequence are those of CD3_sp11a_VH1_VK2_Y.
   420. The CD3 binding molecule of embodiment 343, wherein the heavy chain variable sequence and the corresponding light chain variable sequence are those of CD3_sp11a_VH1_VK2_S.
   421. The CD3 binding molecule of embodiment 343, wherein the heavy chain variable sequence and the corresponding light chain variable sequence are those of CD3_sp11a_VH1_VK2_Y_PTM.
   422. The CD3 binding molecule of embodiment 343, wherein the heavy chain variable sequence and the corresponding light chain variable sequence are those of CD3_sp11a_VH1_VK2_S_PTM.
   423. The CD3 binding molecule of embodiment 343, wherein the heavy chain variable sequence and the corresponding light chain variable sequence are those of CD3_sp11a_VH1_VK2_Y_SW.
   424. The CD3 binding molecule of embodiment 343, wherein the heavy chain variable sequence and the corresponding light chain variable sequence are those of CD3_sp11a_VH1_VK2_S_SW.
   425. The CD3 binding molecule of embodiment 343, wherein the heavy chain variable sequence and the corresponding light chain variable sequence are those of CD3_sp11a_VH1_VK2_Y_PTM.
   426. The CD3 binding molecule of embodiment 343, wherein the heavy chain variable sequence and the corresponding light chain variable sequence are those of CD3_sp11a_VH1_VK2_S_PTM_SW.
   427. The CD3 binding molecule of embodiment 343, wherein the heavy chain variable sequence and the corresponding light chain variable sequence are those of CD3_sp11a_VH1_VK2_SW.
   428. The CD3 binding molecule of embodiment 343, wherein the heavy chain variable sequence and the corresponding light chain variable sequence are those of CD3_sp11a_VH1_VK2_SW PTM.
   429. The CD3 binding molecule of embodiment 343, wherein the heavy chain variable sequence and the corresponding light chain variable sequence are those of CD3_SP11A_VH3_VLK1_Y.
   430. The CD3 binding molecule of embodiment 343, wherein the heavy chain variable sequence and the corresponding light chain variable sequence are those of CD3_SP11A_VH3_VLK1_S.
   431. The CD3 binding molecule of embodiment 343, wherein the heavy chain variable sequence and the corresponding light chain variable sequence are those of CD3_SP11A_VH3_VLK1_Y_PTM.
   432. The CD3 binding molecule of embodiment 343, wherein the heavy chain variable sequence and the corresponding light chain variable sequence are those of CD3_SP11A_VH3_VLK1_S_PTM.
   433. The CD3 binding molecule of embodiment 343, wherein the heavy chain variable sequence and the corresponding light chain variable sequence are those of CD3_SP11A_VH3_VLK1_Y_SW.
   434. The CD3 binding molecule of embodiment 343, wherein the heavy chain variable sequence and the corresponding light chain variable sequence are those of CD3_SP11A_VH3_VLK1_S_W.
   435. The CD3 binding molecule of embodiment 343, wherein the heavy chain variable sequence and the corresponding light chain variable sequence are those of CD3_SP11A_VH3_VLK1_Y_PTM.
   436. The CD3 binding molecule of embodiment 343, wherein the heavy chain variable sequence and the corresponding light chain variable sequence are those of CD3_SP11A_VH3_VLK1_S_PTM_SW.
   437. The CD3 binding molecule of embodiment 343, wherein the heavy chain variable sequence and the corresponding light chain variable sequence are those of CD3_SP11A_VH3_VLK1_PTM_SW.
   438. The CD3 binding molecule of embodiment 343, wherein the heavy chain variable sequence and the corresponding light chain variable sequence are those of CD3_SP11A_VH3_VLK1_SW.
   439. The CD3 binding molecule of embodiment 343, wherein the heavy chain variable sequence and the corresponding light chain variable sequence are those of CD3_SP11A_VH5_VK2_Y.
   440. The CD3 binding molecule of embodiment 343, wherein the heavy chain variable sequence and the corresponding light chain variable sequence are those of CD3_SP11A_VH5_VK2_S.
   441. The CD3 binding molecule of embodiment 343, wherein the heavy chain variable sequence and the corresponding light chain variable sequence are those of CD3_SP11A_VH5_VK2_Y_PTM.
   442. The CD3 binding molecule of embodiment 343, wherein the heavy chain variable sequence and the corresponding light chain variable sequence are those of CD3_SP11A_VH5_VK2_S_PTM.
   443. The CD3 binding molecule of embodiment 343, wherein the heavy chain variable sequence and the corresponding light chain variable sequence are those of CD3_SP11A_VH5_VK2_Y_SW.
   444. The CD3 binding molecule of embodiment 343, wherein the heavy chain variable sequence and the corresponding light chain variable sequence are those of CD3_SP11A_VH5_VK2_S_SW.
   445. The CD3 binding molecule of embodiment 343, wherein the heavy chain variable sequence and the corresponding light chain variable sequence are those of CD3_SP11A_VH5_VK2_Y_PTM_SW.
   446. The CD3 binding molecule of embodiment 343, wherein the heavy chain variable sequence and the corresponding light chain variable sequence are those of CD3_SP11A_VH5_VK2_S_PTM_SW.
   447. The CD3 binding molecule of embodiment 343, wherein the heavy chain variable sequence and the corresponding light chain variable sequence are those of CD3_SP11A_VH5_VK2_PTM_SW.
   448. The CD3 binding molecule of embodiment 343, wherein the heavy chain variable sequence and the corresponding light chain variable sequence are those of CD3_SP11A_VH5_VK2_SW.
   449. The CD3 binding molecule of any one of embodiments 1 to 448, which comprises an antibody, an antibody fragment, an scFv, a dsFv, a Fv, a Fab, an scFab, a (Fab′)2, or a single domain antibody (SDAB).
   450. The CD3 binding molecule of embodiment 449, which comprises an antibody or an antibody fragment.
   451. The CD3 binding molecule of embodiment 449, which comprises a scFv.
   452. The CD3 binding molecule of embodiment 451, wherein the scFv comprises a linker connecting the VH and VL domains.
   453. The CD3 binding molecule of embodiment 452, wherein the linker is 5 to 25 amino acids in length.
   454. The CD3 binding molecule of embodiment 452, wherein the linker is 12 to 20 amino acids in length.
   455. The CD3 binding molecule of embodiment 452, wherein the linker is wherein the linker is selected from any one of linkers L1 through L54.
   456. The CD3 binding molecule of embodiment 452, wherein the linker is linker L24.
   457. The CD3 binding molecule of any one of embodiments 1 to 456, which is a multispecific binding molecule.
   458. The CD3 binding molecule of embodiment 457, which is a bispecific binding molecule (BBM).
   459. The CD3 binding molecule of embodiment 458, wherein the BBM comprises:

(a) an antigen binding module 1 (ABM1) that binds specifically to CD3; and comprises heavy and light chain variable regions of the CD3 binding molecule of any one of embodiments 1 to 448; and

(b) an antigen binding module 2 (ABM2) that binds specifically to a tumor-associated antigen (“TAA”).

460. The CD3 binding molecule of embodiment 459, wherein ABM1 is an antibody, an antibody fragment, an scFv, a dsFv, a Fv, a Fab, an scFab, a (Fab′)2, a single domain antibody (SDAB), a VH or VL domain, or a camelid VHH domain.
461. The CD3 binding molecule of embodiment 460, wherein ABM1 is an scFv.
462. The CD3 binding molecule of embodiment 461, wherein the scFv comprises a linker connecting the VH and VL domains.
463. The CD3 binding molecule of embodiment 462, wherein the linker is 5 to 25 amino acids in length.
464. The CD3 binding molecule of embodiment 462, wherein the linker is 12 to 20 amino acids in length.
465. The CD3 binding molecule of embodiment 462, wherein the linker is wherein the linker is selected from any one of linkers L1 through L54.
466. The CD3 binding molecule of embodiment 462, wherein the linker is linker L24.
467. The CD3 binding molecule of embodiment 460, wherein ABM1 is a Fab.
468. The CD3 binding molecule of embodiment 467, wherein ABM1 is a Fab heterodimer.
469. The CD3 binding molecule of embodiment 460, wherein ABM1 is an antibody or an antigen-binding domain thereof.
470. The CD3 binding molecule of any one of embodiments 459 to 469, wherein ABM2 is an antibody, an antibody fragment, an scFv, a dsFv, a Fv, a Fab, an scFab, a (Fab′)2, a single domain antibody (SDAB), a VH or VL domain, or a camelid VHH domain.
471. The CD3 binding molecule of embodiment 470, wherein ABM2 is an scFv.
472. The CD3 binding molecule of embodiment 470, wherein ABM2 is a Fab.
473. The CD3 binding molecule of embodiment 472, wherein ABM2 is a Fab heterodimer.
474. The CD3 binding molecule of embodiment 470, wherein ABM2 is an antibody or an antigen-binding domain thereof.
475. The CD2 binding molecule of any one of embodiments 459 to 474, in which ABM1 is capable of binding CD3 at the same time ABM2 is bound to its target molecule.
476. The CD3 binding molecule of any one of embodiments 458 to 475, which is bivalent.
477. The CD3 binding molecule of embodiment 476, which has any one of the configurations depicted in FIGS. 1B-1F.
478. The CD3 binding molecule of embodiment 477, which has the configuration depicted in FIG. 1B.
479. The CD3 binding molecule of embodiment 477, which has the configuration depicted in FIG. 10.
480. The CD3 binding molecule of embodiment 477, which has the configuration depicted in FIG. 1D.
481. The CD3 binding molecule of embodiment 477, which has the configuration depicted in FIG. 1E.
482. The CD3 binding molecule of embodiment 477, which has the configuration depicted in FIG. 1F.
483. The CD3 binding molecule of any one of embodiments 477 to 482, which has the configuration referred to as B1 in Section 7.5.1.
484. The CD3 binding molecule of any one of embodiments 477 to 482, which has the configuration referred to as B2 in Section 7.5.1.
485. The CD3 binding molecule of any one of embodiments 458 to 475, which is trivalent.
486. The CD3 binding molecule of embodiment 485, which has any one of the configurations depicted in FIGS. 1G-1Z.
487. The CD3 binding molecule of embodiment 486, which has the configuration depicted in FIG. 1G.
488. The CD3 binding molecule of embodiment 486, which has the configuration depicted in FIG. 1H.
489. The CD3 binding molecule of embodiment 486, which has the configuration depicted in FIG. 11.
490. The CD3 binding molecule of embodiment 486, which has the configuration depicted in FIG. 1J.
491. The CD3 binding molecule of embodiment 486, which has the configuration depicted in FIG. 1K.
492. The CD3 binding molecule of embodiment 486, which has the configuration depicted in FIG. 1L.
493. The CD3 binding molecule of embodiment 486, which has the configuration depicted in FIG. 1M.
494. The CD3 binding molecule of embodiment 486, which has the configuration depicted in FIG. 1N.
495. The CD3 binding molecule of embodiment 486, which has the configuration depicted in FIG. 10.
496. The CD3 binding molecule of embodiment 486, which has the configuration depicted in FIG. 1P.
497. The CD3 binding molecule of embodiment 486, which has the configuration depicted in FIG. 1Q.
498. The CD3 binding molecule of embodiment 486, which has the configuration depicted in FIG. 1R.
499. The CD3 binding molecule of embodiment 486, which has the configuration depicted in FIG. 1S.
500. The CD3 binding molecule of embodiment 486, which has the configuration depicted in FIG. 1T.
501. The CD3 binding molecule of embodiment 486, which has the configuration depicted in FIG. 1U.
502. The CD3 binding molecule of embodiment 486, which has the configuration depicted in FIG. 1V.
503. The CD3 binding molecule of embodiment 486, which has the configuration depicted in FIG. 1W.
504. The CD3 binding molecule of embodiment 486, which has the configuration depicted in FIG. 1X.
505. The CD3 binding molecule of embodiment 486, which has the configuration depicted in FIG. 1Y.
506. The CD3 binding molecule of embodiment 486, which has the configuration depicted in FIG. 1Z.
507. The CD3 binding molecule of any one of embodiments 486 to 506, which has the configuration referred to as T1 in Section 7.5.2.
508. The CD3 binding molecule of any one of embodiments 486 to 506, which has the configuration referred to as T2 in Section 7.5.2.
509. The CD3 binding molecule of any one of embodiments 486 to 506, which has the configuration referred to as T3 in Section 7.5.2.
510. The CD3 binding molecule of any one of embodiments 486 to 506, which has the configuration referred to as T4 in Section 7.5.2.
511. The CD3 binding molecule of any one of embodiments 486 to 506, which has the configuration referred to as T5 in Section 7.5.2.
512. The CD3 binding molecule of any one of embodiments 486 to 506, which has the configuration referred to as T6 in Section 7.5.2.
513. The CD3 binding molecule of any one of embodiments 458 to 475, which is tetravalent.
514. The CD3 binding molecule of embodiment 513, which has any one of the configurations depicted in FIGS. 1AA-1AH.
515. The CD3 binding molecule of embodiment 514, wherein the CD3 binding which has the configuration depicted in FIG. 1AA.
516. The CD3 binding molecule of embodiment 514, which has the configuration depicted in FIG. 1AB.
517. The CD3 binding molecule of embodiment 514, which has the configuration depicted in FIG. 1AC.
518. The CD3 binding molecule of embodiment 514, which has the configuration depicted in FIG. 1AD.
519. The CD3 binding molecule of embodiment 514, which has the configuration depicted in FIG. 1AE.
520. The CD3 binding molecule of embodiment 514, which has the configuration depicted in FIG. 1AF.
521. The CD3 binding molecule of embodiment 514, which has the configuration depicted in FIG. 1AG.
522. The CD3 binding molecule of embodiment 514, which has the configuration depicted in FIG. 1AH.
523. The CD3 binding molecule of any one of embodiments 514 to 522, which has the configuration referred to as Tv1 in Section 7.5.3.
524. The CD3 binding molecule of any one of embodiments 514 to 522, which has the configuration referred to as Tv2 in Section 7.5.3.
525. The CD3 binding molecule of any one of embodiments 514 to 522, which has the configuration referred to as Tv3 in Section 7.5.3.
526. The CD3 binding molecule of any one of embodiments 514 to 522, which has the configuration referred to as Tv4 in Section 7.5.3.
527. The CD3 binding molecule of any one of embodiments 514 to 522, which has the configuration referred to as Tv5 in Section 7.5.3.
528. The CD3 binding molecule of any one of embodiments 514 to 522, which has the configuration referred to as Tv6 in Section 7.5.3.
529. The CD3 binding molecule of any one of embodiments 514 to 522, which has the configuration referred to as Tv7 in Section 7.5.3.
530. The CD3 binding molecule of any one of embodiments 514 to 522, which has the configuration referred to as Tv8 in Section 7.5.3.
531. The CD3 binding molecule of any one of embodiments 514 to 522, which has the configuration referred to as Tv9 in Section 7.5.3.
532. The CD3 binding molecule of any one of embodiments 514 to 522, which has the configuration referred to as Tv10 in Section 7.5.3.
533. The CD3 binding molecule of any one of embodiments 514 to 522, which has the configuration referred to as Tv11 in Section 7.5.3.
534. The CD3 binding molecule of any one of embodiments 514 to 522, which has the configuration referred to as Tv12 in Section 7.5.3.
535. The CD3 binding molecule of any one of embodiments 514 to 522, which has the configuration referred to as Tv13 in Section 7.5.3.
536. The CD3 binding molecule of any one of embodiments 514 to 522, which has the configuration referred to as Tv14 in Section 7.5.3.
537. The CD3 binding molecule of embodiment 457, which is a trispecific binding molecule (TBM).
538. The CD3 binding molecule of embodiment 537, wherein the TBM comprises:

(a) an antigen binding module 1 (ABM1) that binds specifically to CD3 and comprises heavy and light chain variable regions of the CD3 binding molecule of any one of embodiments 1 to 448; and

(b) an antigen binding module 2 (ABM2) that binds specifically to a tumor-associated antigen; and

(c) an antigen binding module 3 (ABM3) that binds specifically to:
(i) a tumor-associated antigen other than the tumor-associated antigen bound by ABM2; or
(ii) CD2.
   539. The CD3 binding molecule of embodiment 538, wherein ABM1 is an antibody, an antibody fragment, an scFv, a dsFv, a Fv, a Fab, an scFab, a (Fab′)2, a single domain antibody (SDAB), a VH or VL domain, or a camelid VHH domain.
   540. The CD3 binding molecule of embodiment 539, wherein ABM1 is an scFv.
   541. The CD3 binding molecule of embodiment 540, wherein the scFv comprises a linker connecting the VH and VL domains.
   542. The CD3 binding molecule of embodiment 541, wherein the linker is 5 to 25 amino acids in length.
   543. The CD3 binding molecule of embodiment 541, wherein the linker is 12 to 20 amino acids in length.
   544. The CD3 binding molecule of embodiment 541, wherein the linker is wherein the linker is selected from any one of linkers L1 through L54.
   545. The CD3 binding molecule of embodiment 541, wherein the linker is linker L24.
   546. The CD3 binding molecule of embodiment 539, wherein ABM1 is a Fab.
   547. The CD3 binding molecule of embodiment 546, wherein ABM1 is a Fab heterodimer.
   548. The CD3 binding molecule of embodiment 539, wherein ABM1 is an antibody or an antigen-binding domain thereof.
   549. The CD3 binding molecule of any one of embodiments 538 to 548, wherein ABM2 is an antibody, an antibody fragment, an scFv, a dsFv, a Fv, a Fab, an scFab, a (Fab′)2, a single domain antibody (SDAB), a VH or VL domain, or a camelid VHH domain.
   550. The CD3 binding molecule of embodiment 549, wherein ABM2 is an scFv.
   551. The CD3 binding molecule of embodiment 549, wherein ABM2 is a Fab.
   552. The CD3 binding molecule of embodiment 551, wherein ABM2 is a Fab heterodimer.
   553. The CD3 binding molecule of embodiment 549, wherein ABM2 is an anti-tumor associated antibody or an antigen-binding domain thereof.
   554. The CD3 binding molecule of any one of embodiments 537 to 553, which is trivalent. 555. The CD3 binding molecule of embodiment 554, which has any one of the configurations depicted in FIGS. 2B-2P.
   556. The CD3 binding molecule of embodiment 555, which has the configuration depicted in FIG. 2B.
   557. The CD3 binding molecule of embodiment 555, which has the configuration depicted in FIG. 2C.
   558. The CD3 binding molecule of embodiment 555, which has the configuration depicted in FIG. 2D.
   559. The CD3 binding molecule of embodiment 555, which has the configuration depicted in FIG. 2E.
   560. The CD3 binding molecule of embodiment 555, which has the configuration depicted in FIG. 2F.
   561. The CD3 binding molecule of embodiment 555, which has the configuration depicted in FIG. 2G.
   562. The CD3 binding molecule of embodiment 555, which has the configuration depicted in FIG. 2H.
   563. The CD3 binding molecule of embodiment 555, which has the configuration depicted in FIG. 2I.
   564. The CD3 binding molecule of embodiment 555, which has the configuration depicted in FIG. 2J.
   565. The CD3 binding molecule of embodiment 555, which has the configuration depicted in FIG. 2K.
   566. The CD3 binding molecule of embodiment 555, which has the configuration depicted in FIG. 2L.
   567. The CD3 binding molecule of embodiment 555, which has the configuration depicted in FIG. 2M.
   568. The CD3 binding molecule of embodiment 555, which has the configuration depicted in FIG. 2N.
   569. The CD3 binding molecule of embodiment 555, which has the configuration depicted in FIG. 2O.
   570. The CD3 binding molecule of embodiment 555, which has the configuration depicted in FIG. 2P.
   571. The CD3 binding molecule of any one of embodiments 554 to 570, which has the configuration referred to as T1 in Section 7.6.1.
   572. CD3 binding molecule of any one of embodiments 554 to 570, which has the configuration referred to as T2 in Section 7.6.1.
   573. CD3 binding molecule of any one of embodiments 554 to 570, which has the configuration referred to as T3 in Section 7.6.1.
   574. CD3 binding molecule of any one of embodiments 554 to 570, which has the configuration referred to as T4 in Section 7.6.1.
   575. CD3 binding molecule of any one of embodiments 554 to 570, which has the configuration referred to as T5 in Section 7.6.1.
   576. CD3 binding molecule of any one of embodiments 554 to 570, which has the configuration referred to as T6 in Section 7.6.1.
   577. The CD3 binding molecule of any one of embodiments 537 to 553, which is tetravalent.
   578. The CD3 binding molecule of embodiment 577, which has any one of the configurations depicted in FIGS. 2Q-2S.
   579. The CD3 binding molecule of embodiment 578, which has the configuration depicted in FIG. 2Q.
   580. The CD3 binding molecule of embodiment 578, which has the configuration depicted in FIG. 2R.
   581. The CD3 binding molecule of embodiment 578, which has the configuration depicted in FIG. 2S.
   582. The CD3 binding molecule of any one of embodiments 577 to 581, which has any of the configurations referred to as Tv1 through Tv24 in Section 7.6.2.
   583. The CD3 binding molecule of any one of embodiments 537 to 553, which is pentavalent.
   584. The CD3 binding molecule of embodiment 583, which has the configuration depicted in FIG. 2T.
   585. The CD3 binding molecule of embodiment 583 or embodiment 584, which has any of the configurations referred to as Pv 1 through Pv 100 in Section 7.6.3.
   586. The CD3 binding molecule of any one of embodiments 537 to 553, which is hexavalent.
   587. The CD3 binding molecule of embodiment 586, which has any one of the configurations depicted in FIGS. 2U-2V.
   588. The CD3 binding molecule of embodiment 587, which has the configuration depicted in FIG. 2U.
   589. The CD3 binding molecule of embodiment 587, which has the configuration depicted in FIG. 2V.
   590. The CD3 binding molecule of any one of embodiments 586 to 589, which has any of the configurations referred to as Hv1 through Hv330 in Section 7.6.4.
   591. The CD3 binding molecule of any one of embodiments 538 to 590, in which ABM1 is capable of binding CD3 at the same time ABM2 and ABM3 are bound to their target molecules.
   592. The CD3 binding molecule of any one of embodiments 459 to 591, wherein ABM2 specifically binds a TAA which is TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, FLT3, TAAG72, CD22, CD33, GD2, BCMA, gp100Tn, FAP, tyrosinase, EPCAM, CEA, Igf-I receptor, EphB2, mesothelin, Cadherin17, CD32b, EGFRvIII, GPNMB, GPR64, HER3, LRP6, LYPD8, NKG2D, SLC34A2, SLC39A6, SLITRK6, or TACSTD2.
   593. The CD3 binding molecule of embodiment 592, wherein ABM2 comprises the CDR sequences of an antibody set forth in Table 15A.
   594. The CD3 binding molecule of embodiment 592, wherein ABM2 comprises the heavy and light chain variable region sequences of an antibody set forth in Table 15A.
   595. The CD3 binding molecule of any one of embodiments 591 to 594, wherein ABM2 binds to CD22.
   596. The CD3 binding molecule of any one of embodiments 591 to 594, wherein ABM2 binds to CS1.
   597. The CD3 binding molecule of any one of embodiments 591 to 594, wherein ABM2 binds to CD33.
   598. The CD3 binding molecule of any one of embodiments 591 to 594, ABM2 binds to GD2.
   599. The CD3 binding molecule of any one of embodiments 591 to 594, wherein ABM2 binds to BCMA.
   600. The CD3 binding molecule of any one of embodiments 591 to 594, wherein ABM2 binds to Tn.
   601. The CD3 binding molecule of any one of embodiments 591 to 594, wherein ABM2 binds to PSMA.
   602. The CD3 binding molecule of any one of embodiments 591 to 594, wherein ABM2 binds to ROR1.
   603. The CD3 binding molecule of any one of embodiments 591 to 594, wherein ABM2 binds to FLT3.
   604. The CD3 binding molecule of any one of embodiments 591 to 594, wherein ABM2 binds to TAAG72.
   605. The CD3 binding molecule of any one of embodiments 591 to 594, wherein ABM2 binds to FAP.
   606. The CD3 binding molecule of any one of embodiments 591 to 594, wherein ABM2 binds to CD38.
   607. The CD3 binding molecule of any one of embodiments 591 to 594, wherein ABM2 binds to CD44v6.
   608. The CD3 binding molecule of any one of embodiments 591 to 594, wherein ABM2 binds to CEA.
   609. The CD3 binding molecule of any one of embodiments 591 to 594, wherein ABM2 binds to EPCAM.
   610. The CD3 binding molecule of any one of embodiments 591 to 594, wherein ABM2 binds to PRSS21.
   611. The CD3 binding molecule of any one of embodiments 591 to 594, wherein ABM2 binds to B7H3.
   612. The CD3 binding molecule of any one of embodiments 591 to 594, wherein ABM2 binds to KIT.
   613. The CD3 binding molecule of any one of embodiments 591 to 594, wherein ABM2 binds to IL-13Ra2.
   614. The CD3 binding molecule of any one of embodiments 591 to 594, wherein ABM2 binds to CD30.
   615. The CD3 binding molecule of any one of embodiments 591 to 594, wherein ABM2 binds to GD3.
   616. The CD3 binding molecule of any one of embodiments 591 to 594, wherein ABM2 binds to CD171.
   617. The CD3 binding molecule of any one of embodiments 591 to 594, wherein ABM2 binds to IL-11Ra.
   618. The CD3 binding molecule of any one of embodiments 591 to 594, wherein ABM2 binds to PSCA.
   619. The CD3 binding molecule of any one of embodiments 591 to 594, wherein ABM2 binds to VEGFR2.
   620. The CD3 binding molecule of any one of embodiments 591 to 594, wherein ABM2 binds to LewisY.
   621. The CD3 binding molecule of any one of embodiments 591 to 594, wherein ABM2 binds to CD24.
   622. The CD3 binding molecule of any one of embodiments 591 to 594, wherein ABM2 binds to PDGFR-beta.
   623. The CD3 binding molecule of any one of embodiments 591 to 594, wherein ABM2 binds to SSEA-4.
   624. The CD3 binding molecule of any one of embodiments 591 to 594, wherein ABM2 binds to CD20.
   625. The CD3 binding molecule of any one of embodiments 591 to 594, wherein ABM2 binds to folate receptor alpha.
   626. The CD3 binding molecule of any one of embodiments 591 to 594, wherein ABM2 binds to ERBB2.
   627. The CD3 binding molecule of any one of embodiments 591 to 594, wherein ABM2 binds to MUC1.
   628. The CD3 binding molecule of any one of embodiments 591 to 594, wherein ABM2 binds to EGFR.
   629. The CD3 binding molecule of any one of embodiments 591 to 594, wherein ABM2 binds to NCAM.
   630. The CD3 binding molecule of any one of embodiments 591 to 594, wherein ABM2 binds to ephrin B2
   631. The CD3 binding molecule of any one of embodiments 591 to 594, wherein ABM2 binds to IGF-I receptor.
   632. The CD3 binding molecule of any one of embodiments 591 to 594, wherein ABM2 binds to CAIX.
   633. The CD3 binding molecule of any one of embodiments 591 to 594, wherein ABM2 binds to LMP2.
   634. The CD3 binding molecule of any one of embodiments 591 to 594, wherein ABM2 binds to gp100.
   635. The CD3 binding molecule of any one of embodiments 591 to 594, wherein ABM2 binds to tyrosinase.
   636. The CD3 binding molecule of any one of embodiments 591 to 594, wherein ABM2 binds to ephA2.
   637. The CD3 binding molecule of any one of embodiments 591 to 594, wherein ABM2 binds to mesothelin.
   638. The CD3 binding molecule of any one of embodiments 591 to 594, wherein ABM2 binds to ALK.
   639. The CD3 binding molecule of any one of embodiments 591 to 594, wherein ABM2 binds to CD19.
   640. The CD3 binding molecule of any one of embodiments 591 to 594, wherein ABM2 binds to CD97.
   641. The CD3 binding molecule of any one of embodiments 591 to 594, wherein ABM2 binds to CLDN6.
   642. The CD3 binding molecule of any one of embodiments 591 to 594, wherein ABM2 binds to EGFRvIII.
   643. The CD3 binding molecule of any one of embodiments 591 to 594, wherein ABM2 binds to folate receptor beta.
   644. The CD3 binding molecule of any one of embodiments 591 to 594, wherein ABM2 binds to GloboH.
   645. The CD3 binding molecule of any one of embodiments 591 to 594, wherein ABM2 binds to GPRC5D. 646. The CD3 binding molecule of any one of embodiments 591 to 594, wherein ABM2 binds to HMWMAA.
   647. The CD3 binding molecule of any one of embodiments 591 to 594, wherein ABM2 binds to LRP6.
   648. The CD3 binding molecule of any one of embodiments 591 to 594, wherein ABM2 binds to NY-BR-1.
   649. The CD3 binding molecule of any one of embodiments 591 to 594, wherein ABM2 binds to PLAC1.
   650. The CD3 binding molecule of any one of embodiments 591 to 594, wherein ABM2 binds to polysialic acid.
   651. The CD3 binding molecule of any one of embodiments 591 to 594, wherein ABM2 binds to TEM1/CD248.
   652. The CD3 binding molecule of any one of embodiments 591 to 594, wherein ABM2 binds to TSHR.
   653. The CD3 binding molecule of any one of embodiments 459 to 591, wherein ABM2 binds to CD19.
   654. The CD3 binding molecule of embodiment 653, wherein ABM2 comprises heavy chain CDRs having the amino acid sequences of CD19-H1, CD19-H2A, and CD19-H3 as set forth in Table 17 and light chain CDRs having the amino acid sequences of CD19-L1, CD19-L2, and CD19-L3 as set forth in Table 17.
   655. The CD3 binding molecule of embodiment 654, wherein ABM2 comprises a heavy chain variable region having the amino acid sequences of VHA as set forth in Table 17 and a light chain variable region having the amino acid sequences of VLA as set forth in Table 17.
   656. The CD3 binding molecule of embodiment 653, wherein ABM2 comprises heavy chain CDRs having the amino acid sequences of CD19-H1, CD19-H2B, and CD19-H3 as set forth in Table 17 and light chain CDRs having the amino acid sequences of CD19-L1, CD19-L2, and CD19-L3 as set forth in Table 17.
   657. The CD3 binding molecule of embodiment 656, wherein ABM2 comprises a heavy chain variable region having the amino acid sequences of VHB as set forth in Table 17 and a light chain variable region having the amino acid sequences of VLB as set forth in Table 17.
   658. The CD3 binding molecule of embodiment 653, wherein ABM2 comprises heavy chain CDRs having the amino acid sequences of CD19-H1, CD19-H2C, and CD19-H3 as set forth in Table 17 and light chain CDRs having the amino acid sequences of CD19-L1, CD19-L2, and CD19-L3 as set forth in Table 17.
   659. The CD3 binding molecule of embodiment 658, wherein ABM2 comprises a heavy chain variable region having the amino acid sequences of VHC as set forth in Table 17 and a light chain variable region having the amino acid sequences of VLB as set forth in Table 17.
   660. The CD3 binding molecule of embodiment 653, wherein ABM2 comprises heavy chain CDRs having the amino acid sequences of CD19-H1, CD19-H2D, and CD19-H3 as set forth in Table 17 and light chain CDRs having the amino acid sequences of CD19-L1, CD19-L2, and CD19-L3 as set forth in Table 17.
   661. The CD3 binding molecule of embodiment 660, wherein ABM2 comprises a heavy chain variable region having the amino acid sequences of VHD as set forth in Table 17 and a light chain variable region having the amino acid sequences of VLB as set forth in Table 17.
   662. The CD3 binding molecule of any one of embodiments 459 to 591, wherein the ABM2 binds BCMA.
   663. The CD3 binding molecule of embodiment 662, wherein ABM2 comprises the CDR sequences of BCMA-1.
   664. The CD3 binding molecule of embodiment 662, wherein ABM2 comprises the CDR sequences of BCMA-2.
   665. The CD3 binding molecule of embodiment 662, wherein ABM2 comprises the CDR sequences of BCMA-3.
   666. The CD3 binding molecule of embodiment 662, wherein ABM2 comprises the CDR sequences of BCMA-4.
   667. The CD3 binding molecule of embodiment 662, wherein ABM2 comprises the CDR sequences of BCMA-5.
   668. The CD3 binding molecule of embodiment 662, wherein ABM2 comprises the CDR sequences of BCMA-6.
   669. The CD3 binding molecule of embodiment 662, wherein ABM2 comprises the CDR sequences of BCMA-7.
   670. The CD3 binding molecule of embodiment 662, wherein ABM2 comprises the CDR sequences of BCMA-8.
   671. The CD3 binding molecule of embodiment 662, wherein ABM2 comprises the CDR sequences of BCMA-9.
   672. The CD3 binding molecule of embodiment 662, wherein ABM2 comprises the CDR sequences of BCMA-10.
   673. The CD3 binding molecule of embodiment 662, wherein ABM2 comprises the CDR sequences of BCMA-11.
   674. The CD3 binding molecule of embodiment 662, wherein ABM2 comprises the CDR sequences of BCMA-12.
   675. The CD3 binding molecule of embodiment 662, wherein ABM2 comprises the CDR sequences of BCMA-13.
   676. The CD3 binding molecule of embodiment 662, wherein ABM2 comprises the CDR sequences of BCMA-14.
   677. The CD3 binding molecule of embodiment 662, wherein ABM2 comprises the CDR sequences of BCMA-15.
   678. The CD3 binding molecule of embodiment 662, wherein ABM2 comprises the CDR sequences of BCMA-16.
   679. The CD3 binding molecule of embodiment 662, wherein ABM2 comprises the CDR sequences of BCMA-17.
   680. The CD3 binding molecule of embodiment 662, wherein ABM2 comprises the CDR sequences of BCMA-18.
   681. The CD3 binding molecule of embodiment 662, wherein ABM2 comprises the CDR sequences of BCMA-19.
   682. The CD3 binding molecule of embodiment 662, wherein ABM2 comprises the CDR sequences of BCMA-20.
   683. The CD3 binding molecule of embodiment 662, wherein ABM2 comprises the CDR sequences of BCMA-21.
   684. The CD3 binding molecule of embodiment 662, wherein ABM2 comprises the CDR sequences of BCMA-22.
   685. The CD3 binding molecule of embodiment 662, wherein ABM2 comprises the CDR sequences of BCMA-23.
   686. The CD3 binding molecule of embodiment 662, wherein ABM2 comprises the CDR sequences of BCMA-24.
   687. The CD3 binding molecule of embodiment 662, wherein ABM2 comprises the CDR sequences of BCMA-25.
   688. The CD3 binding molecule of embodiment 662, wherein ABM2 comprises the CDR sequences of BCMA-26.
   689. The CD3 binding molecule of embodiment 662, wherein ABM2 comprises the CDR sequences of BCMA-27.
   690. The CD3 binding molecule of embodiment 662, wherein ABM2 comprises the CDR sequences of BCMA-28.
   691. The CD3 binding molecule of embodiment 662, wherein ABM2 comprises the CDR sequences of BCMA-29.
   692. The CD3 binding molecule of embodiment 662, wherein ABM2 comprises the CDR sequences of BCMA-30.
   693. The CD3 binding molecule of embodiment 662, wherein ABM2 comprises the CDR sequences of BCMA-31.
   694. The CD3 binding molecule of embodiment 662, wherein ABM2 comprises the CDR sequences of BCMA-32.
   695. The CD3 binding molecule of embodiment 662, wherein ABM2 comprises the CDR sequences of BCMA-33.
   696. The CD3 binding molecule of embodiment 662, wherein ABM2 comprises the CDR sequences of BCMA-34.
   697. The CD3 binding molecule of embodiment 662, wherein ABM2 comprises the CDR sequences of BCMA-35.
   698. The CD3 binding molecule of embodiment 662, wherein ABM2 comprises the CDR sequences of BCMA-36.
   699. The CD3 binding molecule of embodiment 662, wherein ABM2 comprises the CDR sequences of BCMA-37.
   700. The CD3 binding molecule of embodiment 662, wherein ABM2 comprises the CDR sequences of BCMA-38.
   701. The CD3 binding molecule of embodiment 662, wherein ABM2 comprises the CDR sequences of BCMA-39.
   702. The CD3 binding molecule of embodiment 662, wherein ABM2 comprises the CDR sequences of BCMA-40.
   703. The CD3 binding molecule of any one of embodiments 663 to 702, wherein the CDRs are defined by Kabat numbering, as set forth in Table 16B and 16E.
   704. The CD3 binding molecule of any one of embodiments 663 to 702, wherein the CDRs are defined by Chothia numbering, as set forth in Table 16C and 16F.
   705. CD3 binding molecule of any one of embodiments 663 to 702, wherein the CDRs are defined by a combination of Kabat and Chothia numbering, as set forth in Table 16D and 16G.
   706. The CD3 binding molecule of embodiment 662, wherein ABM2 comprises the heavy and light chain variable sequences of BCMA-1, as set forth in Table 16A.
   707. The CD3 binding molecule of embodiment 662, wherein ABM2 comprises the heavy and light chain variable sequences of BCMA-2, as set forth in Table 16A.
   708. The CD3 binding molecule of embodiment 662, wherein ABM2 comprises the heavy and light chain variable sequences of BCMA-3, as set forth in Table 16A.
   709. The CD3 binding molecule of embodiment 662, wherein ABM2 comprises the heavy and light chain variable sequences of BCMA-4, as set forth in Table 16A.
   710. The CD3 binding molecule of embodiment 662, wherein ABM2 comprises the heavy and light chain variable sequences of BCMA-5, as set forth in Table 16A.
   711. The CD3 binding molecule of embodiment 662, wherein ABM2 comprises the heavy and light chain variable sequences of BCMA-6, as set forth in Table 16A.
   712. The CD3 binding molecule of embodiment 662, wherein ABM2 comprises the heavy and light chain variable sequences of BCMA-7, as set forth in Table 16A.
   713. The CD3 binding molecule of embodiment 662, wherein ABM2 comprises the heavy and light chain variable sequences of BCMA-8, as set forth in Table 16A.
   714. The CD3 binding molecule of embodiment 662, wherein ABM2 comprises the heavy and light chain variable sequences of BCMA-9, as set forth in Table 16A.
   715. The CD3 binding molecule of embodiment 662, wherein ABM2 comprises the heavy and light chain variable sequences of BCMA-10, as set forth in Table 16A.
   716. The CD3 binding molecule of embodiment 662, wherein ABM2 comprises the heavy and light chain variable sequences of BCMA-11, as set forth in Table 16A.
   717. The CD3 binding molecule of embodiment 662, wherein ABM2 comprises the heavy and light chain variable sequences of BCMA-12, as set forth in Table 16A.
   718. The CD3 binding molecule of embodiment 662, wherein ABM2 comprises the heavy and light chain variable sequences of BCMA-13, as set forth in Table 16A.
   719. The CD3 binding molecule of embodiment 662, wherein ABM2 comprises the heavy and light chain variable sequences of BCMA-14, as set forth in Table 16A.
   720. The CD3 binding molecule of embodiment 662, wherein ABM2 comprises the heavy and light chain variable sequences of BCMA-15, as set forth in Table 16A.
   721. The CD3 binding molecule of embodiment 662, wherein ABM2 comprises the heavy and light chain variable sequences of BCMA-16, as set forth in Table 16A.
   722. The CD3 binding molecule of embodiment 662, wherein ABM2 comprises the heavy and light chain variable sequences of BCMA-17, as set forth in Table 16A.
   723. The CD3 binding molecule of embodiment 662, wherein ABM2 comprises the heavy and light chain variable sequences of BCMA-18, as set forth in Table 16A.
   724. The CD3 binding molecule of embodiment 662, wherein ABM2 comprises the heavy and light chain variable sequences of BCMA-19, as set forth in Table 16A.
   725. The CD3 binding molecule of embodiment 662, wherein ABM2 comprises the heavy and light chain variable sequences of BCMA-20, as set forth in Table 16A.
   726. The CD3 binding molecule of embodiment 662, wherein ABM2 comprises the heavy and light chain variable sequences of BCMA-21, as set forth in Table 16A.
   727. The CD3 binding molecule of embodiment 662, wherein ABM2 comprises the heavy and light chain variable sequences of BCMA-22, as set forth in Table 16A.
   728. The CD3 binding molecule of embodiment 662, wherein ABM2 comprises the heavy and light chain variable sequences of BCMA-23, as set forth in Table 16A.
   729. The CD3 binding molecule of embodiment 662, wherein ABM2 comprises the heavy and light chain variable sequences of BCMA-24, as set forth in Table 16A.
   730. The CD3 binding molecule of embodiment 662, wherein ABM2 comprises the heavy and light chain variable sequences of BCMA-25, as set forth in Table 16A.
   731. The CD3 binding molecule of embodiment 662, wherein ABM2 comprises the heavy and light chain variable sequences of BCMA-26, as set forth in Table 16A.
   732. The CD3 binding molecule of embodiment 662, wherein ABM2 comprises the heavy and light chain variable sequences of BCMA-27, as set forth in Table 16A.
   733. The CD3 binding molecule of embodiment 662, wherein ABM2 comprises the heavy and light chain variable sequences of BCMA-28, as set forth in Table 16A.
   734. The CD3 binding molecule of embodiment 662, wherein ABM2 comprises the heavy and light chain variable sequences of BCMA-29, as set forth in Table 16A.
   735. The CD3 binding molecule of embodiment 662, wherein ABM2 comprises the heavy and light chain variable sequences of BCMA-30, as set forth in Table 16A.
   736. The CD3 binding molecule of embodiment 662, wherein ABM2 comprises the heavy and light chain variable sequences of BCMA-31, as set forth in Table 16A.
   737. The CD3 binding molecule of embodiment 662, wherein ABM2 comprises the heavy and light chain variable sequences of BCMA-32, as set forth in Table 16A.
   738. The CD3 binding molecule of embodiment 662, wherein ABM2 comprises the heavy and light chain variable sequences of BCMA-33, as set forth in Table 16A.
   739. The CD3 binding molecule of embodiment 662, wherein ABM2 comprises the heavy and light chain variable sequences of BCMA-34, as set forth in Table 16A.
   740. The CD3 binding molecule of embodiment 662, wherein ABM2 comprises the heavy and light chain variable sequences of BCMA-35, as set forth in Table 16A.
   741. The CD3 binding molecule of embodiment 662, wherein ABM2 comprises the heavy and light chain variable sequences of BCMA-36, as set forth in Table 16A.
   742. The CD3 binding molecule of embodiment 662, wherein ABM2 comprises the heavy and light chain variable sequences of BCMA-37, as set forth in Table 16A.
   743. The CD3 binding molecule of embodiment 662, wherein ABM2 comprises the heavy and light chain variable sequences of BCMA-38, as set forth in Table 16A.
   744. The CD3 binding molecule of embodiment 662, wherein ABM2 comprises the heavy and light chain variable sequences of BCMA-39, as set forth in Table 16A.
   745. The CD3 binding molecule of embodiment 662, wherein ABM2 comprises the heavy and light chain variable sequences of BCMA-40, as set forth in Table 16A.
   746. The CD3 binding molecule of any one of embodiments 538 to 745, wherein ABM3 binds CD2.
   747. The CD3 binding molecule of embodiment 746, wherein ABM3 is a non-immunoglobulin scaffold based ABM.
   748. The CD3 binding molecule of embodiment 747, wherein ABM3 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.
   749. The CD3 binding molecule of embodiment 747, wherein ABM3 comprises a receptor binding domain of a CD2 ligand.
   750. The CD3 binding molecule of embodiment 746, wherein ABM3 is a CD58 moiety.
   751. The CD3 binding molecule of embodiment 750, wherein the CD58 moiety comprises the amino acid sequence of CD58-1 as set forth in Table 14.
   752. The CD3 binding molecule of embodiment 750, wherein the CD58 moiety comprises the amino acid sequence of CD58-2 as set forth in Table 14.
   753. The CD3 binding molecule of embodiment 750, wherein the CD58 moiety comprises the amino acid sequence of CD58-3 as set forth in Table 14.
   754. The CD3 binding molecule of embodiment 753, wherein the amino acid designated as B is a phenylalanine.
   755. The CD3 binding molecule of embodiment 753, wherein the amino acid designated as B is a serine.
   756. The CD3 binding molecule of any one of embodiments 753 to 755, wherein the amino acid designated as J is a valine.
   757. The CD3 binding molecule of any one of embodiments 753 to 755, wherein the amino acid designated as J is a lysine.
   758. The CD3 binding molecule of any one of embodiments 753 to 757, wherein the amino acid designated as 0 is a valine.
   759. The CD3 binding molecule of any one of embodiments 753 to 757, wherein the amino acid designated as 0 is a glutamine.
   760. The CD3 binding molecule of any one of embodiments 753 to 759, wherein the amino acid designated as U is a valine.
   761. The CD3 binding molecule of any one of embodiments 753 to 759, wherein the amino acid designated as U is a lysine.
   762. The CD3 binding molecule of any one of embodiments 753 to 761, wherein the amino acid designated as X is a threonine.
   763. The CD3 binding molecule of any one of embodiments 753 to 761, wherein the amino acid designated as X is a serine.
   764. The CD3 binding molecule of any one of embodiments 753 to 763, wherein the amino acid designated as Z is a leucine.
   765. The CD3 binding molecule of any one of embodiments 753 to 763, wherein the amino acid designated as Z is a glycine.
   766. The CD3 binding molecule of embodiment 750, wherein the CD58 moiety comprises the amino acid sequence of CD58-4 as set forth in Table 14.
   767. The CD3 binding molecule of embodiment 750, wherein the CD58 moiety comprises the amino acid sequence of CD58-5 as set forth in Table 14.
   768. The CD3 binding molecule of embodiment 767, wherein the amino acid designated as J is a valine.
   769. The CD3 binding molecule of embodiment 767, wherein the amino acid designated as J is a lysine.
   770. The CD3 binding molecule of any one of embodiments 767 to 769, wherein the amino acid designated as 0 is a valine.
   771. The CD3 binding molecule of any one of embodiments 767 to 769, wherein the amino acid designated as 0 is a glutamine.
   772. The CD3 binding molecule of embodiment 746, wherein ABM3 is a CD48 moiety.
   773. The CD3 binding molecule of embodiment 772, wherein the CD48 moiety has at least 70% sequence identity to amino acids 27-220 of the amino acid sequence of Uniprot identifier P09326.
   774. The CD3 binding molecule of embodiment 772, wherein the CD48 moiety has at least 80% sequence identity to amino acids 27-220 of the amino acid sequence of Uniprot identifier P09326.
   775. The CD3 binding molecule of embodiment 772, wherein the CD48 moiety has at least 90% sequence identity to amino acids 27-220 of the amino acid sequence of Uniprot identifier P09326.
   776. The CD3 binding molecule of embodiment 772, wherein the CD48 moiety has at least 95% sequence identity to amino acids 27-220 of the amino acid sequence of Uniprot identifier P09326.
   777. The CD3 binding molecule of embodiment 772, wherein the CD48 moiety has at least 99% sequence identity to amino acids 27-220 of the amino acid sequence of Uniprot identifier P09326.
   778. The CD3 binding molecule of embodiment 746, wherein ABM3 is an immunoglobulin scaffold based ABM.
   779. The CD3 binding molecule of embodiment 778, wherein ABM3 is an antibody, an antibody fragment, an scFv, a dsFv, a Fv, a Fab, an scFab, a (Fab′)2, a single domain antibody (SDAB), a VH or VL domain, or a camelid VHH domain.
   780. The CD3 binding molecule of embodiment 778, wherein ABM3 is an scFv.
   781. The CD3 binding molecule of embodiment 778, wherein ABM3 is a Fab.
   782. The CD3 binding molecule of embodiment 781, wherein ABM3 is a Fab heterodimer.
   783. The CD3 binding molecule of embodiment 778, wherein ABM3 is an antibody or an antigen-binding domain thereof.
   784. The CD3 binding molecule of any one of embodiments 778 to 783, wherein ABM3 comprises the CDR sequences of CD2-1.
   785. The CD3 binding molecule of embodiment 784, wherein ABM3 comprises the heavy and light chain variable sequences of CD2-1.
   786. The CD3 binding molecule of embodiment 784, wherein ABM3 comprises the heavy and light chain variable sequences of hu1CD2-1.
   787. The CD3 binding molecule of embodiment 784, wherein ABM3 comprises the heavy and light chain variable sequences of hu2CD2-1.
   788. The CD3 binding molecule of embodiment 784, wherein ABM3 comprises the CDR sequences of Medi 507.
   789. The CD3 binding molecule of embodiment 788, wherein ABM3 comprises the heavy and light chain variable sequences of Medi 507.
   790. The CD3 binding molecule of any one of embodiments 538 to 591, wherein ABM2 and ABM3 each bind specifically to a TAA (“TAA 1” and “TAA 2,” respectively).
   791. The CD3 binding molecule of embodiment 790, wherein ABM3 is a non-immunoglobulin scaffold based ABM.
   792. The CD3 binding molecule of embodiment 791, wherein if TAA is a receptor, ABM3 comprises a receptor binding domain of a ligand of the receptor, and if TAA is a ligand, ABM3 comprises a ligand binding domain of a receptor of the ligand.
   793. The CD3 binding molecule of embodiment 791, wherein ABM3 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.
   794. The CD3 binding molecule of embodiment 790, wherein ABM3 is an immunoglobulin scaffold based ABM.
   795. The CD3 binding molecule of embodiment 794, wherein ABM3 is an antibody, an antibody fragment, an scFv, a dsFv, a Fv, a Fab, an scFab, a (Fab′)2, a single domain antibody (SDAB), a VH or VL domain, or a camelid VHH domain.
   796. The CD3 binding molecule of embodiment 795, wherein ABM3 is an antibody or an antigen-binding domain thereof.
   797. The CD3 binding molecule of embodiment 795, wherein ABM3 is an scFv.
   798. The CD3 binding molecule of embodiment 795, wherein ABM3 is a Fab.
   799. The CD3 binding molecule of embodiment 798, wherein ABM3 is a Fab heterodimer.
   800. The CD3 binding molecule of any one of embodiments 790 to 799, wherein TAA 1 and TAA 2 are each independently selected from CD19, CD20, CD22, CD123, BCMA, CD33, CLL1, CD138, CS1, CD38, CD133, FLT3, CD52, TNFRSF13C, TNFRSF13B, CXCR4, PD-L1, LY9, CD200, FCGR2B, CD21, CD23, CD24, CD40L, CD72, CD79a, and CD79b, and optionally wherein ABM2 and/or ABM3 comprise CDR sequences of antibodies set forth in Table 15B, or wherein ABM2 and/or ABM3 comprise the heavy and light chain variable regions of antibodies set forth in Table 15B.
   801. The CD3 binding molecule of embodiment 800, wherein TAA 1 is CD19.
   802. The CD3 binding molecule of embodiment 800, wherein TAA 1 is CD20.
   803. The CD3 binding molecule of embodiment 800, wherein TAA 1 is CD22.
   804. The CD3 binding molecule of embodiment 800, wherein TAA 1 is CD123.
   805. The CD3 binding molecule of embodiment 800, wherein TAA 1 is BCMA.
   806. The CD3 binding molecule of embodiment 800, wherein TAA 1 is CD33.
   807. The CD3 binding molecule of embodiment 800, wherein TAA 1 is CLL1.
   808. The CD3 binding molecule of embodiment 800, wherein TAA 1 is CD138.
   809. The CD3 binding molecule of embodiment 800, wherein TAA 1 is CS1.
   810. The CD3 binding molecule of embodiment 800, wherein TAA 1 is CD38.
   811. The CD3 binding molecule of embodiment 800, wherein TAA 1 is CD133.
   812. The CD3 binding molecule of embodiment 800, wherein TAA 1 is FLT3.
   813. The CD3 binding molecule of embodiment 800, wherein TAA 1 is CD52.
   814. The CD3 binding molecule of embodiment 800, wherein TAA 1 is TNFRSF13C.
   815. The CD3 binding molecule of embodiment 800, wherein TAA 1 is TNFRSF13B.
   816. The CD3 binding molecule of embodiment 800, wherein TAA 1 is CXCR4.
   817. The CD3 binding molecule of embodiment 800, wherein TAA 1 is PD-L1.
   818. The CD3 binding molecule of embodiment 800, wherein TAA 1 is LY9.
   819. The CD3 binding molecule of embodiment 800, wherein TAA 1 is CD200.
   820. The CD3 binding molecule of embodiment 800, wherein TAA 1 is FCGR2B.
   821. The CD3 binding molecule of embodiment 800, wherein TAA 1 is CD21.
   822. The CD3 binding molecule of embodiment 800, wherein TAA 1 is CD23.
   823. The CD3 binding molecule of embodiment 800, wherein TAA 1 is CD24.
   824. The CD3 binding molecule of embodiment 800, wherein TAA 1 is CD40L.
   825. The CD3 binding molecule of embodiment 800, wherein TAA 1 is CD72.
   826. The CD3 binding molecule of embodiment 800, wherein TAA 1 is CD79a.
   827. The CD3 binding molecule of embodiment 800, wherein TAA 1 is CD79b.
   828. The CD3 binding molecule of any one of embodiments 800 or 802 to 827, wherein TAA 2 is CD19.
   829. The CD3 binding molecule of any one of embodiments 800 to 801 or 803 to 827, wherein TAA 2 is CD20.
   830. The CD3 binding molecule of any one of embodiments 800 to 802 or 804 to 827, wherein TAA 2 is CD22.
   831. The CD3 binding molecule of any one of embodiments 800 to 803 or 805 to 827, wherein TAA 2 is CD123.
   832. The CD3 binding molecule of any one of embodiments 800 to 804 or 806 to 827, wherein TAA 2 is BCMA.
   833. The CD3 binding molecule of any one of embodiments 800 to 805 or 807 to 827, wherein TAA 2 is CD33.
   834. The CD3 binding molecule of any one of embodiments 800 to 806 or 808 to 827, wherein TAA 2 is CLL1.
   835. The CD3 binding molecule of any one of embodiments 800 to 807 or 809 to 827, wherein TAA 2 is CD138.
   836. The CD3 binding molecule of any one of embodiments 800 to 808 or 810 to 827, wherein TAA 2 is CS1.
   837. The CD3 binding molecule of any one of embodiments 800 to 809 or 811 to 827, wherein TAA 2 is CD38.
   838. The CD3 binding molecule of any one of embodiments 800 to 810 or 812 to 827, wherein TAA 2 is CD133.
   839. The CD3 binding molecule of any one of embodiments 800 to 811 or 813 to 827, wherein TAA 2 is FLT3.
   840. The CD3 binding molecule of any one of embodiments 800 to 812 or 814 to 827, wherein TAA 2 is CD52.
   841. The CD3 binding molecule of any one of embodiments 800 to 813 or 815 to 827, wherein TAA 2 is TNFRSF13C.
   842. The CD3 binding molecule of any one of embodiments 800 to 814 or 816 to 827, wherein TAA 2 is TNFRSF13B.
   843. The CD3 binding molecule of any one of embodiments 800 to 815 or 817 to 827, wherein TAA 2 is CXCR4.
   844. The CD3 binding molecule of any one of embodiments 800 to 816 or 818 to 827, wherein TAA 2 is PD-L1.
   845. The CD3 binding molecule of any one of embodiments 800 to 817 or 819 to 827, wherein TAA 2 is LY9.
   846. The CD3 binding molecule of any one of embodiments 800 to 818 or 820 to 827, wherein TAA 2 is CD200.
   847. The CD3 binding molecule of any one of embodiments 800 to 819 or 821 to 827, wherein TAA 2 is FCGR2B.
   848. The CD3 binding molecule of any one of embodiments 800 to 820 or 822 to 827, wherein TAA 2 is CD21.
   849. The CD3 binding molecule of any one of embodiments 800 to 821 or 823 to 827, wherein TAA 2 is CD23.
   850. The CD3 binding molecule of any one of embodiments 800 to 822 or 824 to 827, wherein TAA 2 is CD24.
   851. The CD3 binding molecule of any one of embodiments 800 to 823 or 825 to 827, wherein TAA 2 is CD40L.
   852. The CD3 binding molecule of any one of embodiments 800 to 824 or 826 to 827, wherein TAA 2 is CD72.
   853. The CD3 binding molecule of any one of embodiments 800 to 825 or 827, wherein TAA 2 is CD79a.
   854. The CD3 binding molecule of any one of embodiments 800 to 826, wherein TAA 2 is CD79b.
   855. The CD3 binding molecule of any one of embodiments 790 to 854, wherein when ABM2 or ABM3 specifically binds to CD19, ABM2 or ABM3 comprises heavy chain CDRs having the amino acid sequences of CD19-H1, CD19-H2A, and CD19-H3 as set forth in Table 17 and light chain CDRs having the amino acid sequences of CD19-L1, CD19-L2, and CD19-L3 as set forth in Table 17.
   856. The CD3 binding molecule of embodiment 855, wherein ABM2 or ABM3 comprises a heavy chain variable region having the amino acid sequences of VHA as set forth in Table 17 and a light chain variable region having the amino acid sequences of VLA as set forth in Table 17.
   857. The CD3 binding molecule of any one of embodiments 790 to 854, wherein when ABM2 or ABM3 specifically binds to CD19, ABM2 or ABM3 comprises heavy chain CDRs having the amino acid sequences of CD19-H1, CD19-H2B, and CD19-H3 as set forth in Table 17 and light chain CDRs having the amino acid sequences of CD19-L1, CD19-L2, and CD19-L3 as set forth in Table 17.
   858. The CD3 binding molecule of embodiment 857, wherein ABM2 or ABM3 comprises a heavy chain variable region having the amino acid sequences of VHB as set forth in Table 17 and a light chain variable region having the amino acid sequences of VLB as set forth in Table 17.
   859. The CD3 binding molecule of any one of embodiments 790 to 854, wherein when ABM2 or ABM3 specifically binds to CD19, ABM2 or ABM3 comprises heavy chain CDRs having the amino acid sequences of CD19-H1, CD19-H2C, and CD19-H3 as set forth in Table 17 and light chain CDRs having the amino acid sequences of CD19-L1, CD19-L2, and CD19-L3 as set forth in Table 17.
   860. The CD3 binding molecule of embodiment 859, wherein ABM2 or ABM3 comprises a heavy chain variable region having the amino acid sequences of VHC as set forth in Table 17 and a light chain variable region having the amino acid sequences of VLB as set forth in Table 17.
   861. The CD3 binding molecule of any one of embodiments 790 to 854, wherein when ABM2 or ABM3 specifically binds to CD19, ABM2 or ABM3 comprises heavy chain CDRs having the amino acid sequences of CD19-H1, CD19-H2D, and CD19-H3 as set forth in Table 17 and light chain CDRs having the amino acid sequences of CD19-L1, CD19-L2, and CD19-L3 as set forth in Table 17.
   862. The CD3 binding molecule of embodiment 861, wherein ABM2 or ABM3 comprises a heavy chain variable region having the amino acid sequences of VHD as set forth in Table 17 and a light chain variable region having the amino acid sequences of VLB as set forth in Table 17.
   863. The CD3 binding molecule of any one of embodiments 790 to 854, wherein when ABM2 or ABM3 specifically binds to BCMA, ABM2 or ABM3 comprises the CDR sequences of any one of BCMA-1 to BMCA-40.
   864. The CD3 binding molecule of embodiment 863, wherein ABM2 or ABM3 comprises the CDR sequences of BCMA-1.
   865. The CD3 binding molecule of embodiment 863, wherein ABM2 or ABM3 comprises the CDR sequences of BCMA-2.
   866. The CD3 binding molecule of embodiment 863, wherein ABM2 or ABM3 comprises the CDR sequences of BCMA-3.
   867. The CD3 binding molecule of embodiment 863, wherein ABM2 or ABM3 comprises the CDR sequences of BCMA-4.
   868. The CD3 binding molecule of embodiment 863, wherein ABM2 or ABM3 comprises the CDR sequences of BCMA-5.
   869. The CD3 binding molecule of embodiment 863, wherein ABM2 or ABM3 comprises the CDR sequences of BCMA-6.
   870. The CD3 binding molecule of embodiment 863, wherein ABM2 or ABM3 comprises the CDR sequences of BCMA-7.
   871. The CD3 binding molecule of embodiment 863, wherein ABM2 or ABM3 comprises the CDR sequences of BCMA-8.
   872. The CD3 binding molecule of embodiment 863, wherein ABM2 or ABM3 comprises the CDR sequences of BCMA-9.
   873. The CD3 binding molecule of embodiment 863, wherein ABM2 or ABM3 comprises the CDR sequences of BCMA-10.
   874. The CD3 binding molecule of embodiment 863, wherein ABM2 or ABM3 comprises the CDR sequences of BCMA-11.
   875. The CD3 binding molecule of embodiment 863, wherein ABM2 or ABM3 comprises the CDR sequences of BCMA-12.
   876. The CD3 binding molecule of embodiment 863, wherein ABM2 or ABM3 comprises the CDR sequences of BCMA-13.
   877. The CD3 binding molecule of embodiment 863, wherein ABM2 or ABM3 comprises the CDR sequences of BCMA-14.
   878. The CD3 binding molecule of embodiment 863, wherein ABM2 or ABM3 comprises the CDR sequences of BCMA-15.
   879. The CD3 binding molecule of embodiment 863, wherein ABM2 or ABM3 comprises the CDR sequences of BCMA-16.
   880. The CD3 binding molecule of embodiment 863, wherein ABM2 or ABM3 comprises the CDR sequences of BCMA-17.
   881. The CD3 binding molecule of embodiment 863, wherein ABM2 or ABM3 comprises the CDR sequences of BCMA-18.
   882. The CD3 binding molecule of embodiment 863, wherein ABM2 or ABM3 comprises the CDR sequences of BCMA-19.
   883. The CD3 binding molecule of embodiment 863, wherein ABM2 or ABM3 comprises the CDR sequences of BCMA-20.
   884. The CD3 binding molecule of embodiment 863, wherein ABM2 or ABM3 comprises the CDR sequences of BCMA-21.
   885. The CD3 binding molecule of embodiment 863, wherein ABM2 or ABM3 comprises the CDR sequences of BCMA-22.
   886. The CD3 binding molecule of embodiment 863, wherein ABM2 or ABM3 comprises the CDR sequences of BCMA-23.
   887. The CD3 binding molecule of embodiment 863, wherein ABM2 or ABM3 comprises the CDR sequences of BCMA-24.
   888. The CD3 binding molecule of embodiment 863, wherein ABM2 or ABM3 comprises the CDR sequences of BCMA-25.
   889. The CD3 binding molecule of embodiment 863, wherein ABM2 or ABM3 comprises the CDR sequences of BCMA-26.
   890. The CD3 binding molecule of embodiment 863, wherein ABM2 or ABM3 comprises the CDR sequences of BCMA-27.
   891. The CD3 binding molecule of embodiment 863, wherein ABM2 or ABM3 comprises the CDR sequences of BCMA-28.
   892. The CD3 binding molecule of embodiment 863, wherein ABM2 or ABM3 comprises the CDR sequences of BCMA-29.
   893. The CD3 binding molecule of embodiment 863, wherein ABM2 or ABM3 comprises the CDR sequences of BCMA-30.
   894. The CD3 binding molecule of embodiment 863, wherein ABM2 or ABM3 comprises the CDR sequences of BCMA-31.
   895. The CD3 binding molecule of embodiment 863, wherein ABM2 or ABM3 comprises the CDR sequences of BCMA-32.
   896. The CD3 binding molecule of embodiment 863, wherein ABM2 or ABM3 comprises the CDR sequences of BCMA-33.
   897. The CD3 binding molecule of embodiment 863, wherein ABM2 or ABM3 comprises the CDR sequences of BCMA-34.
   898. Th The CD3 binding molecule of embodiment 863, wherein ABM2 or ABM3 comprises the CDR sequences of BCMA-35.
   899. The CD3 binding molecule of embodiment 863, wherein ABM2 or ABM3 comprises the CDR sequences of BCMA-36.
   900. The CD3 binding molecule of embodiment 863, wherein ABM2 or ABM3 comprises the CDR sequences of BCMA-37.
   901. The CD3 binding molecule of embodiment 863, wherein ABM2 or ABM3 comprises the CDR sequences of BCMA-38.
   902. The CD3 binding molecule of embodiment 863, wherein ABM2 or ABM3 comprises the CDR sequences of BCMA-39.
   903. The CD3 binding molecule of embodiment 863, wherein ABM2 or ABM3 comprises the CDR sequences of BCMA-40.
   904. The CD3 binding molecule of any one of embodiments 863 to 903, wherein the CDRs are defined by Kabat numbering, as set forth in Table 16B and 16E.
   905. The CD3 binding molecule of any one of embodiments 863 to 903, wherein the CDRs are defined by Chothia numbering, as set forth in Table 16C and 16F.
   906. CD3 binding molecule of any one of embodiments 863 to 903, wherein the CDRs are defined by a combination of Kabat and Chothia numbering, as set forth in Table 16D and 16G.
   907. The CD3 binding molecule of embodiment 863, wherein ABM2 or ABM3 comprises the heavy and light chain variable sequences of BCMA-1, as set forth in Table 16A.
   908. The CD3 binding molecule of embodiment 863, wherein ABM2 or ABM3 comprises the heavy and light chain variable sequences of BCMA-2, as set forth in Table 16A.
   909. The CD3 binding molecule of embodiment 863, wherein ABM2 or ABM3 comprises the heavy and light chain variable sequences of BCMA-3, as set forth in Table 16A.
   910. The CD3 binding molecule of embodiment 863, wherein ABM2 or ABM3 comprises the heavy and light chain variable sequences of BCMA-4, as set forth in Table 16A.
   911. The CD3 binding molecule of embodiment 863, wherein ABM2 or ABM3 comprises the heavy and light chain variable sequences of BCMA-5, as set forth in Table 16A.
   912. The CD3 binding molecule of embodiment 863, wherein ABM2 or ABM3 comprises the heavy and light chain variable sequences of BCMA-6, as set forth in Table 16A.
   913. The CD3 binding molecule of embodiment 863, wherein ABM2 or ABM3 comprises the heavy and light chain variable sequences of BCMA-7, as set forth in Table 16A.
   914. The CD3 binding molecule of embodiment 863, wherein ABM2 or ABM3 comprises the heavy and light chain variable sequences of BCMA-8, as set forth in Table 16A.
   915. The CD3 binding molecule of embodiment 863, wherein ABM2 or ABM3 comprises the heavy and light chain variable sequences of BCMA-9, as set forth in Table 16A.
   916. The CD3 binding molecule of embodiment 863, wherein ABM2 or ABM3 comprises the heavy and light chain variable sequences of BCMA-10, as set forth in Table 16A.
   917. The CD3 binding molecule of embodiment 863, wherein ABM2 or ABM3 comprises the heavy and light chain variable sequences of BCMA-11, as set forth in Table 16A.
   918. The CD3 binding molecule of embodiment 863, wherein ABM2 or ABM3 comprises the heavy and light chain variable sequences of BCMA-12, as set forth in Table 16A.
   919. The CD3 binding molecule of embodiment 863, wherein ABM2 or ABM3 comprises the heavy and light chain variable sequences of BCMA-13, as set forth in Table 16A.
   920. The CD3 binding molecule of embodiment 863, wherein ABM2 or ABM3 comprises the heavy and light chain variable sequences of BCMA-14, as set forth in Table 16A.
   921. The CD3 binding molecule of embodiment 863, wherein ABM2 or ABM3 comprises the heavy and light chain variable sequences of BCMA-15, as set forth in Table 16A.
   922. The CD3 binding molecule of embodiment 863, wherein ABM2 or ABM3 comprises the heavy and light chain variable sequences of BCMA-16, as set forth in Table 16A.
   923. The CD3 binding molecule of embodiment 863, wherein ABM2 or ABM3 comprises the heavy and light chain variable sequences of BCMA-17, as set forth in Table 16A.
   924. The CD3 binding molecule of embodiment 863, wherein ABM2 or ABM3 comprises the heavy and light chain variable sequences of BCMA-18, as set forth in Table 16A.
   925. The CD3 binding molecule of embodiment 863, wherein ABM2 or ABM3 comprises the heavy and light chain variable sequences of BCMA-19, as set forth in Table 16A.
   926. The CD3 binding molecule of embodiment 863, wherein ABM2 or ABM3 comprises the heavy and light chain variable sequences of BCMA-20, as set forth in Table 16A.
   927. The CD3 binding molecule of embodiment 863, wherein ABM2 or ABM3 comprises the heavy and light chain variable sequences of BCMA-21, as set forth in Table 16A.
   928. The CD3 binding molecule of embodiment 863, wherein ABM2 or ABM3 comprises the heavy and light chain variable sequences of BCMA-22, as set forth in Table 16A.
   929. The CD3 binding molecule of embodiment 863, wherein ABM2 or ABM3 comprises the heavy and light chain variable sequences of BCMA-23, as set forth in Table 16A.
   930. The CD3 binding molecule of embodiment 863, wherein ABM2 or ABM3 comprises the heavy and light chain variable sequences of BCMA-24, as set forth in Table 16A.
   931. The CD3 binding molecule of embodiment 863, wherein ABM2 or ABM3 comprises the heavy and light chain variable sequences of BCMA-25, as set forth in Table 16A.
   932. The CD3 binding molecule of embodiment 863, wherein ABM2 or ABM3 comprises the heavy and light chain variable sequences of BCMA-26, as set forth in Table 16A.
   933. The CD3 binding molecule of embodiment 863, wherein ABM2 or ABM3 comprises the heavy and light chain variable sequences of BCMA-27, as set forth in Table 16A.
   934. The CD3 binding molecule of embodiment 863, wherein ABM2 or ABM3 comprises the heavy and light chain variable sequences of BCMA-28, as set forth in Table 16A.
   935. The CD3 binding molecule of embodiment 863, wherein ABM2 or ABM3 comprises the heavy and light chain variable sequences of BCMA-29, as set forth in Table 16A.
   936. The CD3 binding molecule of embodiment 863, wherein ABM2 or ABM3 comprises the heavy and light chain variable sequences of BCMA-30, as set forth in Table 16A.
   937. The CD3 binding molecule of embodiment 863, wherein ABM2 or ABM3 comprises the heavy and light chain variable sequences of BCMA-31, as set forth in Table 16A.
   938. The CD3 binding molecule of embodiment 863, wherein ABM2 or ABM3 comprises the heavy and light chain variable sequences of BCMA-32, as set forth in Table 16A.
   939. The CD3 binding molecule of embodiment 863, wherein ABM2 or ABM3 comprises the heavy and light chain variable sequences of BCMA-33, as set forth in Table 16A.
   940. The CD3 binding molecule of embodiment 863, wherein ABM2 or ABM3 comprises the heavy and light chain variable sequences of BCMA-34, as set forth in Table 16A.
   941. The CD3 binding molecule of embodiment 863, wherein ABM2 or ABM3 comprises the heavy and light chain variable sequences of BCMA-35, as set forth in Table 16A.
   942. The CD3 binding molecule of embodiment 863, wherein ABM2 or ABM3 comprises the heavy and light chain variable sequences of BCMA-36, as set forth in Table 16A.
   943. The CD3 binding molecule of embodiment 863, wherein ABM2 or ABM3 comprises the heavy and light chain variable sequences of BCMA-37, as set forth in Table 16A.
   944. The CD3 binding molecule of embodiment 863, wherein ABM2 or ABM3 comprises the heavy and light chain variable sequences of BCMA-38, as set forth in Table 16A.
   945. The CD3 binding molecule of embodiment 863, wherein ABM2 or ABM3 comprises the heavy and light chain variable sequences of BCMA-39, as set forth in Table 16A.
   946. The CD3 binding molecule of embodiment 863, wherein ABM2 or ABM3 comprises the heavy and light chain variable sequences of BCMA-40, as set forth in Table 16A.
   947. The CD3 binding molecule of any one of embodiments 1 to 946, which comprises a first variant Fc region and a second variant Fc region that together form an Fc heterodimer.
   948. The CD3 binding molecule of embodiment 947, wherein the first and second variant Fc regions comprise the amino acid substitutions S364K/E357Q:L368D/K370S.
   949. The CD3 binding molecule of any one of embodiments 947, wherein the first and second variant Fc regions comprise the amino acid substitutions L368D/K370S:S364.
   950. The CD3 binding molecule of any one of embodiments 947 to 949, wherein the first and second variant Fc regions comprise the amino acid substitutions L368E/K370S:S364K.
   951. The CD3 binding molecule of embodiment 947, wherein the first and second variant Fc regions comprise the amino acid substitutions T411T/E360E/Q362E:D401K.
   952. The CD3 binding molecule of embodiment 947, wherein the first and second variant Fc regions comprise the amino acid substitutions L368D 370S:S364/E357L.
   953. The CD3 binding molecule of embodiment 947, wherein the first and second variant Fc regions comprise the amino acid substitutions 370S:S364K/E357Q.
   954. The CD3 binding molecule of embodiment 947, wherein the first and second variant Fc regions comprise the amino acid substitutions of any one of the steric variants listed in FIG. 4 of WO 2014/110601 (reproduced in Table 4).
   955. The CD3 binding molecule of embodiment 947, wherein the first and second variant Fc regions comprise the amino acid substitutions of any one of the variants listed in FIG. 5 of WO 2014/110601 (reproduced in Table 4).
   956. The CD3 binding molecule of embodiment 947, wherein the first and second variant Fc regions comprise the amino acid substitutions of any one of the variants listed in FIG. 6 of WO 2014/110601 (reproduced in Table 4).
   957. The CD3 binding molecule of any one of embodiments 947 to 956, wherein at least one of the Fc regions comprises an ablation variant modification.
   958. The CD3 binding molecule of embodiment 957, wherein the ablation variant modifications are selected from Table 3.
   959. The CD3 binding molecule of embodiment 958, wherein the ablation variant modification comprises G236R.
   960. The CD3 binding molecule of embodiment 958, wherein the ablation variant modification comprises S239G.
   961. The CD3 binding molecule of embodiment 958, wherein the ablation variant modification comprises S239K.
   962. The CD3 binding molecule of embodiment 958, wherein the ablation variant modification comprises S239Q.
   963. The CD3 binding molecule of embodiment 958, wherein the ablation variant modification comprises S239R.
   964. The CD3 binding molecule of embodiment 958, wherein the ablation variant modification comprises V266D.
   965. The CD3 binding molecule of embodiment 958, wherein the ablation variant modification comprises S267K.
   966. The CD3 binding molecule of embodiment 958, wherein the ablation variant modification comprises S267R.
   967. The CD3 binding molecule of embodiment 958, wherein the ablation variant modification comprises H268K.
   968. The CD3 binding molecule of embodiment 958, wherein the ablation variant modification comprises E269R.
   969. The CD3 binding molecule of embodiment 958, wherein the ablation variant modification comprises 299R.
   970. The CD3 binding molecule of embodiment 958, wherein the ablation variant modification comprises 299K
   971. The CD3 binding molecule of embodiment 958, wherein the ablation variant modification comprises K322A
   972. The CD3 binding molecule of embodiment 958, wherein the ablation variant modification comprises A327G
   973. The CD3 binding molecule of embodiment 958, wherein the ablation variant modification comprises A327L
   974. The CD3 binding molecule of embodiment 958, wherein the ablation variant modification comprises A327N
   975. The CD3 binding molecule of embodiment 958, wherein the ablation variant modification comprises A327Q
   976. The CD3 binding molecule of embodiment 958, wherein the ablation variant modification comprises L328E
   977. The CD3 binding molecule of embodiment 958, wherein the ablation variant modification comprises L328R
   978. The CD3 binding molecule of embodiment 958, wherein the ablation variant modification comprises P329A
   979. The CD3 binding molecule of embodiment 958, wherein the ablation variant modification comprises P329H
   980. The CD3 binding molecule of embodiment 958, wherein the ablation variant modification comprises P329K
   981. The CD3 binding molecule of embodiment 958, wherein the ablation variant modification comprises A330L
   982. The CD3 binding molecule of embodiment 958, wherein the ablation variant modification comprises A330S/P331S
   983. The CD3 binding molecule of embodiment 958, wherein the ablation variant modification comprises 1332K
   984. The CD3 binding molecule of embodiment 958, wherein the ablation variant modification comprises 1332R
   985. The CD3 binding molecule of embodiment 958, wherein the ablation variant modification comprises V266D/A327Q
   986. The CD3 binding molecule of embodiment 958, wherein the ablation variant modification comprises V266D/P329K
   987. The CD3 binding molecule of embodiment 958, wherein the ablation variant modification comprises G236R/L328R
   988. The CD3 binding molecule of embodiment 958, wherein the ablation variant modification comprises E233P/L234V/L235A/G236del/S239K.
   989. The CD3 binding molecule of embodiment 958, wherein the ablation variant modification comprises E233P/L234V/L235A/G236del/S267K.
   990. The CD3 binding molecule of embodiment 958, wherein the ablation variant modification comprises E233P/L234V/L235A/G236del/S239K/A327G.
   991. The CD3 binding molecule of embodiment 958, wherein the ablation variant modification comprises E233P/L234V/L235A/G236del/S267K/A327G.
   992. The CD3 binding molecule of embodiment 958, wherein the ablation variant modification comprises E233P/L234V/L235A/G236del.
   993. The CD3 binding molecule of embodiment 958, wherein the ablation variant modification comprises S239K/S267K.
   994. The CD3 binding molecule of embodiment 958, wherein the ablation variant modification comprises 267K/P329K.
   995. The CD3 binding molecule of any one of embodiments 957 to 994, wherein both variant Fc regions comprise the ablation variant modification.
   996. The CD3 binding molecule of any one of embodiments 947 to 995, wherein at least one of the Fc regions further comprises pl variant substitutions.
   997. The CD3 binding molecule of embodiment 996 wherein the pl variant substitutions are selected from Table 6.
   998. The CD3 binding molecule of embodiment 997, wherein the pl variant substitutions comprise the substitutions present in pl_ISO(−).
   999. The CD3 binding molecule of embodiment 997, wherein the pl variant substitutions comprise the substitutions present in pl_(−)_isosteric_A.
   1000. The CD3 binding molecule of embodiment 997, wherein the pl variant substitutions comprise the substitutions present in pl_(−)_isosteric_B.
   1001. The CD3 binding molecule of embodiment 997, wherein the pl variant substitutions comprise the substitutions present in PL_ISO(+RR).
   1002. The CD3 binding molecule of embodiment 997, wherein the pl variant substitutions comprise the substitutions present in pl_ISO(+).
   1003. The CD3 binding molecule of embodiment 997, wherein the pl variant substitutions comprise the substitutions present in pl_(+)_isosteric_A.
   1004. The CD3 binding molecule of embodiment 997, wherein the pl variant substitutions comprise the substitutions present in pl_(+)_isosteric_B.
   1005. The CD3 binding molecule of embodiment 997, wherein the pl variant substitutions comprise the substitutions present in pl_(+)_isosteric_E269Q/E272Q.
   1006. The CD3 binding molecule of embodiment 997, wherein the pl variant substitutions comprise the substitutions present in pl_(+)_isosteric_E269Q/E283Q.
   1007. The CD3 binding molecule of embodiment 997, wherein the pl variant substitutions comprise the substitutions present in pl_(+)_isosteric_E2720/E283Q.
   1008. The CD3 binding molecule of embodiment 997, wherein the pl variant substitutions comprise the substitutions present in pl_(+)_isosteric_E269Q.
   1009. The CD3 binding molecule of any one of embodiments 947 to 1008, wherein the first and/or second Fc region further comprises one or more amino acid substitution(s) selected from 434A, 434S, 428L, 308F, 259I, 428L/434S, 259I/308F, 436I/428L, 4361 or V/434S, 436V/428L, 252Y, 252Y/254T/256E, 2591/308F/428L, 236A, 239D, 239E, 332E, 332D, 239D/332E, 267D, 267E, 328F, 267E/328F, 236A/332E, 239D/332E/330Y, 239D, 332E/330L, 236R, 328R, 236R/328R, 236N/267E, 243L, 298A and 299T.
   1010. The CD3 binding molecule of any one of embodiments 947 to 1008, wherein the first and/or second Fc region further comprises one or more amino acid substitution comprises the amino acid substitution 434A, 434S or 434V.
   1011. The CD3 binding molecule of embodiment 1010, wherein the first and/or second Fc region further comprises one or more amino acid substitution comprises the amino acid substitution 428L.
   1012. The CD3 binding molecule of any one of embodiments 1010 to 1011, wherein the first and/or second Fc region further comprises one or more amino acid substitution comprises the amino acid substitution 308F.
   1013. The CD3 binding molecule of any one of embodiments 1010 to 1012, wherein the first and/or second Fc region further comprises one or more amino acid substitution comprises the amino acid substitution 259I.
   1014. The CD3 binding molecule of any one of embodiments 1010 to 1013, wherein the first and/or second Fc region further comprises one or more amino acid substitution comprises the amino acid substitution 436I.
   1015. The CD3 binding molecule of any one of embodiments 1010 to 1014, wherein the first and/or second Fc region further comprises one or more amino acid substitution comprises the amino acid substitution 252Y.
   1016. The CD3 binding molecule of any one of embodiments 1010 to 1015, wherein the first and/or second Fc region further comprises one or more amino acid substitution comprises the amino acid substitution 254T.
   1017. The CD3 binding molecule of any one of embodiments 1010 to 1016, wherein the first and/or second Fc region further comprises one or more amino acid substitution comprises the amino acid substitution 256E.
   1018. The CD3 binding molecule of any one of embodiments 1010 to 1017, wherein the first and/or second Fc region further comprises one or more amino acid substitution comprises the amino acid substitution 239D or 239E.
   1019. The CD3 binding molecule of any one of embodiments 1010 to 1018, wherein the first and/or second Fc region further comprises one or more amino acid substitution comprises the amino acid substitution 332E or 332D.
   1020. The CD3 binding molecule of any one of embodiments 1010 to 1019, wherein the first and/or second Fc region further comprises one or more amino acid substitution comprises the amino acid substitution 267D or 267E.
   1021. The CD3 binding molecule of any one of embodiments 1010 to 1020, wherein the first and/or second Fc region further comprises one or more amino acid substitution comprises the amino acid substitution 330L.
   1022. The CD3 binding molecule of any one of embodiments 1010 to 1021, wherein the first and/or second Fc region further comprises one or more amino acid substitution comprises the amino acid substitution 236R or 236N.
   1023. The CD3 binding molecule of any one of embodiments 1010 to 1022, wherein the first and/or second Fc region further comprises one or more amino acid substitution comprises the amino acid substitution 328R.
   1024. The CD3 binding molecule of any one of embodiments 1010 to 1023, wherein the first and/or second Fc region further comprises one or more amino acid substitution comprises the amino acid substitution 243L.
   1025. The CD3 binding molecule of any one of embodiments 1010 to 1024, wherein the first and/or second Fc region further comprises one or more amino acid substitution comprises the amino acid substitution 298A.
   1026. The CD3 binding molecule of any one of embodiments 1010 to 1025, wherein the first and/or second Fc region further comprises one or more amino acid substitution comprises the amino acid substitution 299T.
   1027. The CD3 binding molecule of embodiment 947, wherein:

(a) the first and second variant Fc regions comprise the amino acid substitutions S364K/E357Q:L368D/K370S;

(b) the first and/or second variant Fc regions comprises the ablation variant modifications E233P/L234V/L235A/G236del/S267K, and

(c) the first and/or second variant Fc regions comprises the pl variant substitutions N208D/Q295E/N384D/Q418E/N421D (pl_(−)_isosteric_A).

1028. The CD3 binding molecule of embodiment 1027, wherein the first variant Fc region comprises the ablation variant modifications E233P/L234V/L235A/G236del/S267K.
1029. The CD3 binding molecule of any one of embodiments 1027 to 1028, wherein the second variant Fc region comprises the ablation variant modifications E233P/L234V/L235A/G236del/S267K.
1030. The CD3 binding molecule of any one of embodiments 1027 to 1029, wherein the first variant Fc region comprises the pl variant substitutions N208D/Q295E/N384D/Q418E/N421D (pl_(−)_isosteric_A).
1031. The CD3 binding molecule of any one of embodiments 1027 to 1030, wherein the second variant Fc region comprises the pl variant substitutions N208D/Q295E/N384D/Q418E/N421D (pl_(−)_isosteric_A).
1032. The CD3 binding molecule of any one of embodiments 1 to 946, which comprises an Fc domain.
1033. The CD3 binding molecule of embodiment 1032, wherein the Fc domain is an Fc heterodimer.
1034. The CD3 binding molecule of embodiment 1033, wherein the Fc heterodimer comprises any of the Fc modifications set forth in Table 4. 1035. The CD3 binding molecule of embodiment 1033, wherein the Fc heterodimer comprises knob-in-hole (“KIH”) modifications.
1036. The CD3 binding molecule of any one of embodiments to 1033 to 1035, which comprises at least one of the Fc modifications designated as Fc 1 through Fc 150.
1037. The CD3 binding molecule of embodiment 1036, which comprises at least one of the Fc modifications designated as Fc 1 through Fc 5.
1038. The CD3 binding molecule of embodiment 1036, which comprises at least one of the Fc modifications designated as Fc 6 through Fc 10.
1039. The CD3 binding molecule of embodiment 1036, which comprises at least one of the Fc modifications designated as Fc 11 through Fc 15.
1040. The CD3 binding molecule of embodiment 1036, which comprises at least one of the Fc modifications designated as Fc 16 through Fc 20.
1041. The CD3 binding molecule of embodiment 1036, which comprises at least one of the Fc modifications designated as Fc 21 through Fc 25.
1042. The CD3 binding molecule of embodiment 1036, which comprises at least one of the Fc modifications designated as Fc 26 through Fc 30.
1043. The CD3 binding molecule of embodiment 1036, which comprises at least one of the Fc modifications designated as Fc 31 through Fc 35.
1044. The CD3 binding molecule of embodiment 1036, which comprises at least one of the Fc modifications designated as Fc 36 through Fc 40.
1045. The CD3 binding molecule of embodiment 1036, which comprises at least one of the Fc modifications designated as Fc 41 through Fc 45.
1046. The CD3 binding molecule of embodiment 1036, which comprises at least one of the Fc modifications designated as Fc 46 through Fc 50.
1047. The CD3 binding molecule of embodiment 1036, which comprises at least one of the Fc modifications designated as Fc 51 through Fc 55.
1048. The CD3 binding molecule of embodiment 1036, which comprises at least one of the Fc modifications designated as Fc 56 through Fc 60.
1049. The CD3 binding molecule of embodiment 1036, which comprises at least one of the Fc modifications designated as Fc 61 through Fc 65.
1050. The CD3 binding molecule of embodiment 1036, which comprises at least one of the Fc modifications designated as Fc 66 through Fc 70.
1051. The CD3 binding molecule of embodiment 1036, which comprises at least one of the Fc modifications designated as Fc 71 through Fc 75.
1052. The CD3 binding molecule of embodiment 1036, which comprises at least one of the Fc modifications designated as Fc 76 through Fc 80.
1053. The CD3 binding molecule of embodiment 1036, which comprises at least one of the Fc modifications designated as Fc 81 through Fc 85.
1054. The CD3 binding molecule of embodiment 1036, which comprises at least one of the Fc modifications designated as Fc 86 through Fc 90.
1055. The CD3 binding molecule of embodiment 1036, which comprises at least one of the Fc modifications designated as Fc 91 through Fc 95.
1056. The CD3 binding molecule of embodiment 1036, which comprises at least one of the Fc modifications designated as Fc 96 through Fc 100.
1057. The CD3 binding molecule of embodiment 1036, which comprises at least one of the Fc modifications designated as Fc 101 through Fc 105.
1058. The CD3 binding molecule of embodiment 1036, which comprises at least one of the Fc modifications designated as Fc 106 through Fc 110.
1059. The CD3 binding molecule of embodiment 1036, which comprises at least one of the Fc modifications designated as Fc 111 through Fc 115.
1060. The CD3 binding molecule of embodiment 1036, which comprises at least one of the Fc modifications designated as Fc 116 through Fc 120.
1061. The CD3 binding molecule of embodiment 1036, which comprises at least one of the Fc modifications designated as Fc 121 through Fc 125.
1062. The CD3 binding molecule of embodiment 1036, which comprises at least one of the Fc modifications designated as Fc 126 through Fc 130.
1063. The CD3 binding molecule of embodiment 1036, which comprises at least one of the Fc modifications designated as Fc 131 through Fc 135.
1064. The CD3 binding molecule of embodiment 1036, which comprises at least one of the Fc modifications designated as Fc 136 through Fc 140.
1065. The CD3 binding molecule of embodiment 1036, which comprises at least one of the Fc modifications designated as Fc 141 through Fc 145.
1066. The CD3 binding molecule of embodiment 1036, which comprises at least one of the Fc modifications designated as Fc 146 through Fc 150.
1067. The CD3 binding molecule of any one of embodiments 1032 to 1066, wherein the Fc domain has altered effector function.
1068. The CD3 binding molecule of embodiment 1067, wherein the Fc domain has altered binding to one or more Fc receptors.
1069. The CD3 binding molecule of embodiment 1068, wherein the one or more Fc receptors comprise FcRN.
1070. The CD3 binding molecule of embodiment 1068 or embodiment 1069, wherein the one or more Fc receptors comprise leukocyte receptors.
1071. The CD3 binding molecule of any one of embodiments 1032 to 1070, wherein the Fc has modified disulfide bond architecture.
1072. The CD3 binding molecule of any one of embodiments 1032 to 1071, wherein the Fc has altered glycosylation patterns.
1073. The CD3 binding molecule of any one of embodiments 1032 to 1072, wherein the Fc comprises a hinge region.
1074. The CD3 binding molecule of embodiment 1073, wherein the hinge region comprises any one of the hinge regions described in Section 7.4.2
1075. The CD3 binding molecule of embodiment 1074, wherein the hinge region comprises the amino acid sequence of the hinge region designated H1.
1076. The CD3 binding molecule of embodiment 1074, wherein the hinge region comprises the amino acid sequence of the hinge region designated H2.
1077. The CD3 binding molecule of embodiment 1074, wherein the hinge region comprises the amino acid sequence of the hinge region designated H3.
1078. The CD3 binding molecule of embodiment 1074, wherein the hinge region comprises the amino acid sequence of the hinge region designated H4.
1079. The CD3 binding molecule of embodiment 1074, wherein the hinge region comprises the amino acid sequence of the hinge region designated H5.
1080. The CD3 binding molecule of embodiment 1074, wherein the hinge region comprises the amino acid sequence of the hinge region designated H6.
1081. The CD3 binding molecule of embodiment 1074, wherein the hinge region comprises the amino acid sequence of the hinge region designated H7.
1082. The CD3 binding molecule of embodiment 1074, wherein the hinge region comprises the amino acid sequence of the hinge region designated H8.
1083. The CD3 binding molecule of embodiment 1074, wherein the hinge region comprises the amino acid sequence of the hinge region designated H9.
1084. The CD3 binding molecule of embodiment 1074, wherein the hinge region comprises the amino acid sequence of the hinge region designated H10.
1085. The CD3 binding molecule of embodiment 1074, wherein the hinge region comprises the amino acid sequence of the hinge region designated H11.
1086. The CD3 binding molecule of embodiment 1074, wherein the hinge region comprises the amino acid sequence of the hinge region designated H12.
1087. The CD3 binding molecule of embodiment 1074, wherein the hinge region comprises the amino acid sequence of the hinge region designated H13.
1088. The CD3 binding molecule of embodiment 1074, wherein the hinge region comprises the amino acid sequence of the hinge region designated H14.
1089. The CD3 binding molecule of embodiment 1074, wherein the hinge region comprises the amino acid sequence of the hinge region designated H15.
1090. The CD3 binding molecule of embodiment 1074, wherein the hinge region comprises the amino acid sequence of the hinge region designated H16.
1091. The CD3 binding molecule of embodiment 1074, wherein the hinge region comprises the amino acid sequence of the hinge region designated H17.
1092. The CD3 binding molecule of embodiment 1074, wherein the hinge region comprises the amino acid sequence of the hinge region designated H18.
1093. The CD3 binding molecule of embodiment 1074, wherein the hinge region comprises the amino acid sequence of the hinge region designated H19.
1094. The CD3 binding molecule of embodiment 1074, wherein the hinge region comprises the amino acid sequence of the hinge region designated H20.
1095. The CD3 binding molecule of embodiment 1074, wherein the hinge region comprises the amino acid sequence of the hinge region designated H21.
1096. The CD3 binding molecule of any one of embodiments 1 to 1095, which comprises at least one scFv domain.
1097. The CD3 binding molecule of embodiment 1096, wherein at least one scFv comprises a linker connecting the VH and VL domains.
1098. The CD3 binding molecule of embodiment 1097, wherein the linker is 5 to 25 amino acids in length.
1099. The CD3 binding molecule of embodiment 1098, wherein the linker is 12 to 20 amino acids in length.
1100. The CD3 binding molecule of any one of embodiments 1097 to 1099, wherein the linker is a charged linker and/or a flexible linker.
1101. The CD3 binding molecule of any one of embodiments 1097 to 1100, wherein the linker is selected from any one of linkers L1 through L54.
1102. The CD3 binding molecule of embodiment 1101, wherein the linker region comprises the amino acid sequence of the linker designated L1.
1103. The CD3 binding molecule of embodiment 1101, wherein the linker region comprises the amino acid sequence of the linker designated L2.
1104. The CD3 binding molecule of embodiment 1101, wherein the linker region comprises the amino acid sequence of the linker designated L3.
1105. The CD3 binding molecule of embodiment 1101, wherein the linker region comprises the amino acid sequence of the linker designated L4.
1106. The CD3 binding molecule of embodiment 1101, wherein the linker region comprises the amino acid sequence of the linker designated L5.
1107. The CD3 binding molecule of embodiment 1101, wherein the linker region comprises the amino acid sequence of the linker designated L6.
1108. The CD3 binding molecule of embodiment 1101, wherein the linker region comprises the amino acid sequence of the linker designated L7.
1109. The CD3 binding molecule of embodiment 1101, wherein the linker region comprises the amino acid sequence of the linker designated L8.
1110. The CD3 binding molecule of embodiment 1101, wherein the linker region comprises the amino acid sequence of the linker designated L9.
1111. The CD3 binding molecule of embodiment 1101, wherein the linker region comprises the amino acid sequence of the linker designated L10.
1112. The CD3 binding molecule of embodiment 1101, wherein the linker region comprises the amino acid sequence of the linker designated L11.
1113. The CD3 binding molecule of embodiment 1101, wherein the linker region comprises the amino acid sequence of the linker designated L12.
1114. The CD3 binding molecule of embodiment 1101, wherein the linker region comprises the amino acid sequence of the linker designated L13.
1115. The CD3 binding molecule of embodiment 1101, wherein the linker region comprises the amino acid sequence of the linker designated L14.
1116. The CD3 binding molecule of embodiment 1101, wherein the linker region comprises the amino acid sequence of the linker designated L15.
1117. The CD3 binding molecule of embodiment 1101, wherein the linker region comprises the amino acid sequence of the linker designated L16.
1118. The CD3 binding molecule of embodiment 1101, wherein the linker region comprises the amino acid sequence of the linker designated L17.
1119. The CD3 binding molecule of embodiment 1101, wherein the linker region comprises the amino acid sequence of the linker designated L18.
1120. The CD3 binding molecule of embodiment 1101, wherein the linker region comprises the amino acid sequence of the linker designated L19.
1121. The CD3 binding molecule of embodiment 1101, wherein the linker region comprises the amino acid sequence of the linker designated L20.
1122. The CD3 binding molecule of embodiment 1101, wherein the linker region comprises the amino acid sequence of the linker designated L21.
1123. The CD3 binding molecule of embodiment 1101, wherein the linker region comprises the amino acid sequence of the linker designated L22.
1124. The CD3 binding molecule of embodiment 1101, wherein the linker region comprises the amino acid sequence of the linker designated L23.
1125. The CD3 binding molecule of embodiment 1101, wherein the linker region comprises the amino acid sequence of the linker designated L24.
1126. The CD3 binding molecule of embodiment 1101, wherein the linker region comprises the amino acid sequence of the linker designated L25.
1127. The CD3 binding molecule of embodiment 1101, wherein the linker region comprises the amino acid sequence of the linker designated L26.
1128. The CD3 binding molecule of embodiment 1101, wherein the linker region comprises the amino acid sequence of the linker designated L27.
1129. The CD3 binding molecule of embodiment 1101, wherein the linker region comprises the amino acid sequence of the linker designated L28.
1130. The CD3 binding molecule of embodiment 1101, wherein the linker region comprises the amino acid sequence of the linker designated L29.
1131. The CD3 binding molecule of embodiment 1101, wherein the linker region comprises the amino acid sequence of the linker designated L30.
1132. The CD3 binding molecule of embodiment 1101, wherein the linker region comprises the amino acid sequence of the linker designated L31.
1133. The CD3 binding molecule of embodiment 1101, wherein the linker region comprises the amino acid sequence of the linker designated L32.
1134. The CD3 binding molecule of embodiment 1101, wherein the linker region comprises the amino acid sequence of the linker designated L33.
1135. The CD3 binding molecule of embodiment 1101, wherein the linker region comprises the amino acid sequence of the linker designated L34.
1136. The CD3 binding molecule of embodiment 1101, wherein the linker region comprises the amino acid sequence of the linker designated L35.
1137. The CD3 binding molecule of embodiment 1101, wherein the linker region comprises the amino acid sequence of the linker designated L36.
1138. The CD3 binding molecule of embodiment 1101, wherein the linker region comprises the amino acid sequence of the linker designated L37.
1139. The CD3 binding molecule of embodiment 1101, wherein the linker region comprises the amino acid sequence of the linker designated L38.
1140. The CD3 binding molecule of embodiment 1101, wherein the linker region comprises the amino acid sequence of the linker designated L39.
1141. The CD3 binding molecule of embodiment 1101, wherein the linker region comprises the amino acid sequence of the linker designated L40.
1142. The CD3 binding molecule of embodiment 1101, wherein the linker region comprises the amino acid sequence of the linker designated L41.
1143. The CD3 binding molecule of embodiment 1101, wherein the linker region comprises the amino acid sequence of the linker designated L42.
1144. The CD3 binding molecule of embodiment 1101, wherein the linker region comprises the amino acid sequence of the linker designated L43.
1145. The CD3 binding molecule of embodiment 1101, wherein the linker region comprises the amino acid sequence of the linker designated L44.
1146. The CD3 binding molecule of embodiment 1101, wherein the linker region comprises the amino acid sequence of the linker designated L45.
1147. The CD3 binding molecule of embodiment 1101, wherein the linker region comprises the amino acid sequence of the linker designated L46.
1148. The CD3 binding molecule of embodiment 1101, wherein the linker region comprises the amino acid sequence of the linker designated L47.
1149. The CD3 binding molecule of embodiment 1101, wherein the linker region comprises the amino acid sequence of the linker designated L48.
1150. The CD3 binding molecule of embodiment 1101, wherein the linker region comprises the amino acid sequence of the linker designated L49.
1151. The CD3 binding molecule of embodiment 1101, wherein the linker region comprises the amino acid sequence of the linker designated L50.
1152. The CD3 binding molecule of embodiment 1101, wherein the linker region comprises the amino acid sequence of the linker designated L51.
1153. The CD3 binding molecule of embodiment 1101, wherein the linker region comprises the amino acid sequence of the linker designated L52.
1154. The CD3 binding molecule of embodiment 1101, wherein the linker region comprises the amino acid sequence of the linker designated L53.
1155. The CD3 binding molecule of embodiment 1101, wherein the linker region comprises the amino acid sequence of the linker designated L54.
1156. The CD3 binding molecule of any one of embodiments 1 to 1155, which comprises at least one Fab domain.
1157. The CD3 binding molecule of embodiment 1156, wherein at least one Fab domain comprises any of the Fab heterodimerization modifications set forth in Table 2.
1158. The CD3 binding molecule of embodiment 1157, wherein at least one Fab domain comprises the Fab heterodimerization modifications designated as F1.
1159. The CD3 binding molecule of embodiment 1157, wherein at least one Fab domain comprises the Fab heterodimerization modifications designated as F2.
1160. The CD3 binding molecule of embodiment 1157, wherein at least one Fab domain comprises the Fab heterodimerization modifications designated as F3.
1161. The CD3 binding molecule of embodiment 1157, wherein at least one Fab domain comprises the Fab heterodimerization modifications designated as F4.
1162. The CD3 binding molecule of embodiment 1157, wherein at least one Fab domain comprises the Fab heterodimerization modifications designated as F5.
1163. The CD3 binding molecule of embodiment 1157, wherein at least one Fab domain comprises the Fab heterodimerization modifications designated as F6.
1164. The CD3 binding molecule of embodiment 1157, wherein at least one Fab domain comprises the Fab heterodimerization modifications designated as F7.
1165. The CD3 binding molecule of any one of embodiments 1 to 1164, which comprises at least two ABMs, an ABM and an ABM chain, or two ABM chains connected to one another via a linker.
1166. The CD3 binding molecule of embodiment 1165, wherein the linker is 5 to 25 amino acids in length.
1167. The CD3 binding molecule of embodiment 1166, wherein the linker is 12 to 20 amino acids in length.
1168. The CD3 binding molecule of any one of embodiments 1165 to 1167, wherein the linker is a charged linker and/or a flexible linker.
1169. The CD3 binding molecule of any one of embodiments 1165 to 1168, wherein the linker is selected from any one of linkers L1 through L54.
1170. The CD3 binding molecule of embodiment 1169, wherein the linker region comprises the amino acid sequence of the linker designated L1.
1171. The CD3 binding molecule of embodiment 1169, wherein the linker region comprises the amino acid sequence of the linker designated L2.
1172. The CD3 binding molecule of embodiment 1169, wherein the linker region comprises the amino acid sequence of the linker designated L3.
1173. The CD3 binding molecule of embodiment 1169, wherein the linker region comprises the amino acid sequence of the linker designated L4.
1174. The CD3 binding molecule of embodiment 1169, wherein the linker region comprises the amino acid sequence of the linker designated L5.
1175. The CD3 binding molecule of embodiment 1169, wherein the linker region comprises the amino acid sequence of the linker designated L6.
1176. The CD3 binding molecule of embodiment 1169, wherein the linker region comprises the amino acid sequence of the linker designated L7.
1177. The CD3 binding molecule of embodiment 1169, wherein the linker region comprises the amino acid sequence of the linker designated L8.
1178. The CD3 binding molecule of embodiment 1169, wherein the linker region comprises the amino acid sequence of the linker designated L9.
1179. The CD3 binding molecule of embodiment 1169, wherein the linker region comprises the amino acid sequence of the linker designated L10.
1180. The CD3 binding molecule of embodiment 1169, wherein the linker region comprises the amino acid sequence of the linker designated L11.
1181. The CD3 binding molecule of embodiment 1169, wherein the linker region comprises the amino acid sequence of the linker designated L12.
1182. The CD3 binding molecule of embodiment 1169, wherein the linker region comprises the amino acid sequence of the linker designated L13.
1183. The CD3 binding molecule of embodiment 1169, wherein the linker region comprises the amino acid sequence of the linker designated L14.
1184. The CD3 binding molecule of embodiment 1169, wherein the linker region comprises the amino acid sequence of the linker designated L15.
1185. The CD3 binding molecule of embodiment 1169, wherein the linker region comprises the amino acid sequence of the linker designated L16.
1186. The CD3 binding molecule of embodiment 1169, wherein the linker region comprises the amino acid sequence of the linker designated L17.
1187. The CD3 binding molecule of embodiment 1169, wherein the linker region comprises the amino acid sequence of the linker designated L18.
1188. The CD3 binding molecule of embodiment 1169, wherein the linker region comprises the amino acid sequence of the linker designated L19.
1189. The CD3 binding molecule of embodiment 1169, wherein the linker region comprises the amino acid sequence of the linker designated L20.
1190. The CD3 binding molecule of embodiment 1169, wherein the linker region comprises the amino acid sequence of the linker designated L21.
1191. The CD3 binding molecule of embodiment 1169, wherein the linker region comprises the amino acid sequence of the linker designated L22.
1192. The CD3 binding molecule of embodiment 1169, wherein the linker region comprises the amino acid sequence of the linker designated L23.
1193. The CD3 binding molecule of embodiment 1169, wherein the linker region comprises the amino acid sequence of the linker designated L24.
1194. The CD3 binding molecule of embodiment 1169, wherein the linker region comprises the amino acid sequence of the linker designated L25.
1195. The CD3 binding molecule of embodiment 1169, wherein the linker region comprises the amino acid sequence of the linker designated L26.
1196. The CD3 binding molecule of embodiment 1169, wherein the linker region comprises the amino acid sequence of the linker designated L27.
1197. The CD3 binding molecule of embodiment 1169, wherein the linker region comprises the amino acid sequence of the linker designated L28.
1198. The CD3 binding molecule of embodiment 1169, wherein the linker region comprises the amino acid sequence of the linker designated L29.
1199. The CD3 binding molecule of embodiment 1169, wherein the linker region comprises the amino acid sequence of the linker designated L30.
1200. The CD3 binding molecule of embodiment 1169, wherein the linker region comprises the amino acid sequence of the linker designated L31.
1201. The CD3 binding molecule of embodiment 1169, wherein the linker region comprises the amino acid sequence of the linker designated L32.
1202. The CD3 binding molecule of embodiment 1169, wherein the linker region comprises the amino acid sequence of the linker designated L33.
1203. The CD3 binding molecule of embodiment 1169, wherein the linker region comprises the amino acid sequence of the linker designated L34.
1204. The CD3 binding molecule of embodiment 1169, wherein the linker region comprises the amino acid sequence of the linker designated L35.
1205. The CD3 binding molecule of embodiment 1169, wherein the linker region comprises the amino acid sequence of the linker designated L36.
1206. The CD3 binding molecule of embodiment 1169, wherein the linker region comprises the amino acid sequence of the linker designated L37.
1207. The CD3 binding molecule of embodiment 1169, wherein the linker region comprises the amino acid sequence of the linker designated L38.
1208. The CD3 binding molecule of embodiment 1169, wherein the linker region comprises the amino acid sequence of the linker designated L39.
1209. The CD3 binding molecule of embodiment 1169, wherein the linker region comprises the amino acid sequence of the linker designated L40.
1210. The CD3 binding molecule of embodiment 1169, wherein the linker region comprises the amino acid sequence of the linker designated L41.
1211. The CD3 binding molecule of embodiment 1169, wherein the linker region comprises the amino acid sequence of the linker designated L42.
1212. The CD3 binding molecule of embodiment 1169, wherein the linker region comprises the amino acid sequence of the linker designated L43.
1213. The CD3 binding molecule of embodiment 1169, wherein the linker region comprises the amino acid sequence of the linker designated L44.
1214. The CD3 binding molecule of embodiment 1169, wherein the linker region comprises the amino acid sequence of the linker designated L45.
1215. The CD3 binding molecule of embodiment 1169, wherein the linker region comprises the amino acid sequence of the linker designated L46.
1216. The CD3 binding molecule of embodiment 1169, wherein the linker region comprises the amino acid sequence of the linker designated L47.
1217. The CD3 binding molecule of embodiment 1169, wherein the linker region comprises the amino acid sequence of the linker designated L48.
1218. The CD3 binding molecule of embodiment 1169, wherein the linker region comprises the amino acid sequence of the linker designated L49.
1219. The CD3 binding molecule of embodiment 1101, wherein the linker region comprises the amino acid sequence of the linker designated L50.
1220. The CD3 binding molecule of embodiment 1101, wherein the linker region comprises the amino acid sequence of the linker designated L51.
1221. The CD3 binding molecule of embodiment 1101, wherein the linker region comprises the amino acid sequence of the linker designated L52.
1222. The CD3 binding molecule of embodiment 1101, wherein the linker region comprises the amino acid sequence of the linker designated L53.
1223. The CD3 binding molecule of embodiment 1169, wherein the linker region comprises the amino acid sequence of the linker designated L54.
1224. The CD3 binding molecule of any one of embodiments 1 to 1223 for use as a medicament.
1225. A conjugate comprising the CD3 binding molecule of any one of embodiments 1 to 1223 and an agent.
1226. The conjugate of embodiment 1225, wherein the agent is a therapeutic agent, a diagnostic agent, a masking moiety, a cleavable moiety, a stabilizing agent, or any combination thereof.
1227. The conjugate of embodiment 1225, wherein the agent is any of the agents described in Section 7.13.
1228. The conjugate of any one of embodiments 1225 to 1227, wherein the CD3 binding molecule is conjugated to a radionuclide.
1229. The conjugate of any one of embodiments 1225 to 1227, wherein the CD3 binding molecule is conjugated to an alkylating agent.
1230. The conjugate of any one of embodiments 1225 to 1227, wherein the CD3 binding molecule is conjugated to a topoisomerase inhibitor, which is optionally a topoisomerase I inhibitor or a topoisomerase II inhibitor.
1231. The conjugate of any one of embodiments 1225 to 1227, wherein the CD3 binding molecule is conjugated to a DNA damaging agent.
1232. The conjugate of any one of embodiments 1225 to 1227, wherein the CD3 binding molecule is conjugated to a DNA intercalating agent, optionally a groove binding agent such as a minor groove binding agent.
1233. The conjugate of any one of embodiments 1225 to 1227, wherein the CD3 binding molecule is conjugated to a RNA/DNA antimetabolite.
1234. The conjugate of any one of embodiments 1225 to 1227, wherein the CD3 binding molecule is conjugated to a kinase inhibitor.
1235. The conjugate of any one of embodiments 1225 to 1227, wherein the CD3 binding molecule is conjugated to a protein synthesis inhibitor.
1236. The conjugate of any one of embodiments 1225 to 1227, wherein the CD3 binding molecule is conjugated to a histone deacetylase (HDAC) inhibitor.
1237. The conjugate of any one of embodiments 1225 to 1227, wherein the CD3 binding molecule is conjugated to a mitochondrial inhibitor, which is optionally an inhibitor of a phosphoryl transfer reaction in mitochondria.
1238. The conjugate of any one of embodiments 1225 to 1227, wherein the CD3 binding molecule is conjugated to an antimitotic agent.
1239. The conjugate of any one of embodiments 1225 to 1227, wherein the CD3 binding molecule is conjugated to a maytansinoid.
1240. The conjugate of any one of embodiments 1225 to 1227, wherein the CD3 binding molecule is conjugated to a kinesin inhibitor.
1241. The conjugate of any one of embodiments 1225 to 1227, wherein the CD3 binding molecule is conjugated to a kinesin-like protein KIF11 inhibitor.
1242. The conjugate of any one of embodiments 1225 to 1227, wherein the CD3 binding molecule is conjugated to a V-ATPase (vacuolar-type H+-ATPase) inhibitor.
1243. The conjugate of any one of embodiments 1225 to 1227, wherein the CD3 binding molecule is conjugated to a pro-apoptotic agent.
1244. The conjugate of any one of embodiments 1225 to 1227, wherein the CD3 binding molecule is conjugated to a Bcl2 (B-cell lymphoma 2) inhibitor.
1245. The conjugate of any one of embodiments 1225 to 1227, wherein the CD3 binding molecule is conjugated to a MCL1 (myeloid cell leukemia 1) inhibitor.
1246. The conjugate of any one of embodiments 1225 to 1227, wherein the CD3 binding molecule is conjugated to a HSP90 (heat shock protein 90) inhibitor.
1247. The conjugate of any one of embodiments 1225 to 1227, wherein the CD3 binding molecule is conjugated to an IAP (inhibitor of apoptosis) inhibitor.
1248. The conjugate of any one of embodiments 1225 to 1227, wherein the CD3 binding molecule is conjugated to a mTOR (mechanistic target of rapamycin) inhibitor.
1249. The conjugate of any one of embodiments 1225 to 1227, wherein the CD3 binding molecule is conjugated to a microtubule stabilizer.
1250. The conjugate of any one of embodiments 1225 to 1227, wherein the CD3 binding molecule is conjugated to a microtubule destabilizer.
1251. The conjugate of any one of embodiments 1225 to 1227, wherein the CD3 binding molecule is conjugated to an auristatin.
1252. The conjugate of any one of embodiments 1225 to 1227, wherein the CD3 binding molecule is conjugated to a dolastatin.
1253. The conjugate of any one of embodiments 1225 to 1227, wherein the CD3 binding molecule is conjugated to a MetAP (methionine aminopeptidase).
1254. The conjugate of any one of embodiments 1225 to 1227, wherein the CD3 binding molecule is conjugated to a CRM1 (chromosomal maintenance 1) inhibitor.
1255. The conjugate of any one of embodiments 1225 to 1227, wherein the CD3 binding molecule is conjugated to a DPPIV (dipeptidyl peptidase IV) inhibitor.
1256. The conjugate of any one of embodiments 1225 to 1227, wherein the CD3 binding molecule is conjugated to a proteasome inhibitor.
1257. The conjugate of any one of embodiments 1225 to 1227, wherein the CD3 binding molecule is conjugated to a protein synthesis inhibitor.
1258. The conjugate of any one of embodiments 1225 to 1227, wherein the CD3 binding molecule is conjugated to a CDK2 (cyclin-dependent kinase 2) inhibitor.
1259. The conjugate of any one of embodiments 1225 to 1227, wherein the CD3 binding molecule is conjugated to a CDK9 (cyclin-dependent kinase 9) inhibitor.
1260. The conjugate of any one of embodiments 1225 to 1227, wherein the CD3 binding molecule is conjugated to a RNA polymerase inhibitor.
1261. The conjugate of any one of embodiments 1225 to 1227, wherein the CD3 binding molecule is conjugated to a DHFR (dihydrofolate reductase) inhibitor.
1262. The conjugate of any one of embodiments 1225 to 1261, wherein the agent is attached to the TBM with a linker, which is optionally a cleavable linker or a non-cleavable linker, e.g., a linker as described in Section 7.13.2.
1263. A pharmaceutical composition comprising the CD3 binding molecule of any one of embodiments 1 to 1224 or the conjugate of any one of embodiments 1225 to 1262 and a pharmaceutically acceptable excipient.
1264. A method of activating T cells in a subject in need thereof, comprising administering to the subject an effective amount of the CD3 binding molecule of any one of embodiments 1 to 1224, the conjugate of any one of embodiments 1225 to 1262 or the pharmaceutical composition of embodiment 1263.
1265. The method of embodiment 1264, wherein the subject has a proliferative disease.
1266. The method of embodiment 1265, wherein the proliferative disease is a cancer or a precancerous condition.
1267. The method of embodiment 1265 or 1266, wherein the proliferative disease is a hematologic proliferative disease.
1268. The method of 1267, wherein the proliferative disease is a lymphoma, a leukemia, multiple myeloma, a chronic myeloproliferative neoplasm, a macroglobulinemia, a myelodysplastic syndrome, a myelodysplastic/myeloproliferative neoplasm, or a plasmacytic dendritic cell neoplasm.
1269. The method of embodiment 1268, wherein the proliferative disease is a lymphoma.
1270. The method of embodiment 1269, wherein the lymphoma is Hodgkin's lymphoma.
1271. The method of embodiment 1270, wherein the Hodgkin's lymphoma is nodular sclerosing Hodgkin's lymphoma, mixed-cellularity subtype Hodgkin's lymphoma, lymphocyte-rich or lymphocytic predominance Hodgkin's lymphoma, or lymphocyte depleted Hodgkin's lymphoma.
1272. The method of embodiment 1271, wherein the Hodgkin's lymphoma is nodular sclerosing Hodgkin's lymphoma.
1273. The method of embodiment 1271, wherein the Hodgkin's lymphoma is mixed-cellularity subtype Hodgkin's lymphoma.
1274. The method of embodiment 1271, wherein the Hodgkin's lymphoma is lymphocyte-rich or lymphocytic predominance Hodgkin's lymphoma.
1275. The method of embodiment 1271, wherein the Hodgkin's lymphoma is lymphocyte depleted Hodgkin's lymphoma.
1276. The method of embodiment 1269, wherein the lymphoma is non-Hodgkin's lymphoma.
1277. The method of embodiment 1276, wherein the non-Hodgkin's lymphoma is a B cell lymphoma or a T cell lymphoma.
1278. The method of embodiment 1277, wherein the non-Hodgkin's lymphoma is a B cell lymphoma.
1279. The method of embodiment 1277, wherein the non-Hodgkin's lymphoma is a T cell lymphoma
1280. The method of embodiment 1276, wherein the non-Hodgkin's lymphoma is diffuse large B-cell lymphoma (DLBCL), follicular lymphoma, chronic lymphocytic leukemia (CLL)/small lymphocytic lymphoma (SLL), mantle cell lymphoma (MCL), marginal zone lymphoma, Burkitt lymphoma, lymphoplasmacytic lymphoma (Waldenstrom macroglobulinemia), primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, primary effusion lymphoma, anaplastic large cell lymphoma (ALCL), adult T-cell lymphoma, angiocentric lymphoma, angioimmunoblastic T-cell lymphoma, cutaneous T-cell lymphoma, extranodal natural killer/T-cell lymphoma, enteropathy type intestinal T-cell lymphoma, precursor T-lymphoblastic lymphoma, or unspecified peripheral T-cell lymphoma.
1281. The method of embodiment 1280, wherein the non-Hodgkin's lymphoma is diffuse large B-cell lymphoma (DLBCL).
1282. The method of embodiment 1280, wherein the non-Hodgkin's lymphoma is follicular lymphoma.
1283. The method of embodiment 1280, wherein the non-Hodgkin's lymphoma is chronic lymphocytic leukemia (CLL)/small lymphocytic lymphoma (SLL).
1284. The method of embodiment 1280, wherein the non-Hodgkin's lymphoma is mantle cell lymphoma (MCL), marginal zone lymphoma.
1285. The method of embodiment 1280, wherein the non-Hodgkin's lymphoma is Burkitt lymphoma.
1286. The method of embodiment 1280, wherein the non-Hodgkin's lymphoma is lymphoplasmacytic lymphoma (Waldenstrom macroglobulinemia).
1287. The method of embodiment 1280, wherein the non-Hodgkin's lymphoma is primary central nervous system (CNS) lymphoma.
1288. The method of embodiment 1280, wherein the non-Hodgkin's lymphoma is primary mediastinal large B-cell lymphoma.
1289. The method of embodiment 1280, wherein the non-Hodgkin's lymphoma is mediastinal grey-zone lymphoma (MGZL).
1290. The method of embodiment 1280, wherein the non-Hodgkin's lymphoma is splenic marginal zone B-cell lymphoma.
1291. The method of embodiment 1280, wherein the non-Hodgkin's lymphoma is extranodal marginal zone B-cell lymphoma of MALT.
1292. The method of embodiment 1280, wherein the non-Hodgkin's lymphoma is nodal marginal zone B-cell lymphoma.
1293. The method of embodiment 1280, wherein the non-Hodgkin's lymphoma is primary effusion lymphoma, anaplastic large cell lymphoma (ALCL).
1294. The method of embodiment 1280, wherein the non-Hodgkin's lymphoma is adult T-cell lymphoma.
1295. The method of embodiment 1280, wherein the non-Hodgkin's lymphoma is angiocentric lymphoma.
1296. The method of embodiment 1280, wherein the non-Hodgkin's lymphoma is angioimmunoblastic T-cell lymphoma.
1297. The method of embodiment 1280, wherein the non-Hodgkin's lymphoma is cutaneous T-cell lymphoma.
1298. The method of embodiment 1280, wherein the non-Hodgkin's lymphoma is extranodal natural killer/T-cell lymphoma.
1299. The method of embodiment 1280, wherein the non-Hodgkin's lymphoma is enteropathy type intestinal T-cell lymphoma.
1300. The method of embodiment 1280, wherein the non-Hodgkin's lymphoma is precursor T-lymphoblastic lymphoma.
1301. The method of embodiment 1280, wherein the non-Hodgkin's lymphoma is unspecified peripheral T-cell lymphoma.
1302. The method of embodiment 1268, wherein the proliferative disease is a leukemia.
1303. The method of embodiment 1302, wherein the leukemia is B-cell acute lymphoid leukemia (BALL), T-cell acute lymphoid leukemia (TALL), acute lymphoid leukemia (ALL), acute myeloid leukemia (AML), chronic myelogenous leukemia (CML), chronic lymphocytic leukemia (CLL), B-cell chronic lymphocytic leukemia (B-CLL), B-cell prolymphocytic leukemia (B-PLL), hairy cell leukemia, precursor B-lymphoblastic leukemia (PB-LBL), large granular lymphocyte leukemia, precursor T-lymphoblastic leukemia (T-LBL), or T-cell chronic lymphocytic leukemia/prolymphocytic leukemia (T-CLL/PLL).
1304. The method of embodiment 1303, wherein the leukemia is B-cell acute lymphoid leukemia (BALL).
1305. The method of embodiment 1303, wherein the leukemia is T-cell acute lymphoid leukemia (TALL).
1306. The method of embodiment 1303, wherein the leukemia is acute lymphoid leukemia (ALL).
1307. The method of embodiment 1303, wherein the leukemia is acute myeloid leukemia (AML).
1308. The method of embodiment 1303, wherein the leukemia is chronic myelogenous leukemia (CML).
1309. The method of embodiment 1303, wherein the leukemia is chronic lymphocytic leukemia (CLL).
1310. The method of embodiment 1303, wherein the leukemia is B-cell chronic lymphocytic leukemia (B-CLL).
1311. The method of embodiment 1303, wherein the leukemia is B-cell prolymphocytic leukemia (B-PLL).
1312. The method of embodiment 1303, wherein the leukemia is hairy cell leukemia.
1313. The method of embodiment 1303, wherein the leukemia is precursor B-lymphoblastic leukemia (PB-LBL).
1314. The method of embodiment 1303, wherein the leukemia is large granular lymphocyte leukemia.
1315. The method of embodiment 1303, wherein the leukemia is precursor T-lymphoblastic leukemia (T-LBL).
1316. The method of embodiment 1303, wherein the leukemia is T-cell chronic lymphocytic leukemia/prolymphocytic leukemia (T-CLL/PLL).
1317. The method of embodiment 1268, wherein the proliferative disease is multiple myeloma.
1318. The method of embodiment 1268, wherein the proliferative disease is a chronic myeloproliferative neoplasm.
1319. The method of embodiment 1268, wherein the proliferative disease is a macroglobulinemia.
1320. The method of embodiment 1268, wherein the proliferative disease is a myelodysplastic syndrome.
1321. The method of embodiment 1268, wherein the proliferative disease is a myelodysplastic/myeloproliferative neoplasm.
1322. The method of embodiment 1268, wherein the proliferative disease is a plasmacytic dendritic cell neoplasm
1323. The method of embodiment 1265 or 1266, wherein the proliferative disease is adrenocortical carcinoma, anal cancer, appendix cancer, bile duct cancer, bladder cancer, bone cancer, brain cancer, breast cancer, bronchial tumor, carcinoma of unknown primary origin, cervical cancer, a chordoma, colon cancer, colorectal cancer, embryonal tumor, endometrial cancer, ependymoma, esophageal cancer, esthesioneuroblastoma, Ewing sarcoma, eye cancer, malignant fibrous histiocytoma, germ cell tumor, gallbladder cancer, gastric cancer, gastrointestinal carcinoid tumor, gastrointestinal stromal tumor, gestational trophoblastic disease, glioma, head and neck cancer, heart cancer, HER2+ cancer, hypopharyngeal cancer, Kaposi sarcoma, kidney cancer, Langerhans cell histiocytosis, laryngeal cancer, lip and oral cavity cancer, liver cancer, lung cancer, mesothelioma, metastatic squamous neck cancer with occult primary, midline tract carcinoma involving NUT gene, mouth cancer, nasal cavity cancer, nasopharyngeal cancer, neuroblastoma, oropharyngeal cancer, ovarian cancer, pancreatic cancer, para-nasal sinus cancer, paraganglioma, parathyroid cancer, penile cancer, pharyngeal cancer, pituitary cancer, pleuropulmonary blastoma, prostate cancer, rectal cancer, renal cell cancer, renal pelvis and ureter cancer, retinoblastoma, a rhabdoid tumor, salivary gland cancer, skin cancer, small intestine cancer, soft tissue sarcoma, spinal cord tumor, stomach cancer, teratoid tumor, testicular cancer, throat cancer, thymoma, thymic carcinoma, thyroid cancer, urethral cancer, uterine cancer, vaginal cancer, vulvar cancer, or Wilms tumor.
1324. The method of embodiment 1323, wherein the proliferative disease is adrenocortical carcinoma.
1325. The method of embodiment 1323, wherein the proliferative disease is anal cancer.
1326. The method of embodiment 1323, wherein the proliferative disease is appendix cancer.
1327. The method of embodiment 1323, wherein the proliferative disease is bile duct cancer.
1328. The method of embodiment 1323, wherein the proliferative disease is bladder cancer.
1329. The method of embodiment 1323, wherein the proliferative disease is bone cancer.
1330. The method of embodiment 1323, wherein the proliferative disease is brain cancer.
1331. The method of embodiment 1323, wherein the proliferative disease is breast cancer.
1332. The method of embodiment 1323, wherein the proliferative disease is bronchial tumor.
1333. The method of embodiment 1323, wherein the proliferative disease is carcinoma of unknown primary origin.
1334. The method of embodiment 1323, wherein the proliferative disease is cervical cancer.
1335. The method of embodiment 1323, wherein the proliferative disease is a chordoma.
1336. The method of embodiment 1323, wherein the proliferative disease is colon cancer.
1337. The method of embodiment 1323, wherein the proliferative disease is colorectal cancer.
1338. The method of embodiment 1323, wherein the proliferative disease is embryonal tumor.
1339. The method of embodiment 1323, wherein the proliferative disease is endometrial cancer.
1340. The method of embodiment 1323, wherein the proliferative disease is ependymoma.
1341. The method of embodiment 1323, wherein the proliferative disease is esophageal cancer.
1342. The method of embodiment 1323, wherein the proliferative disease is esthesioneuroblastoma.
1343. The method of embodiment 1323, wherein the proliferative disease is Ewing sarcoma.
1344. The method of embodiment 1323, wherein the proliferative disease is eye cancer.
1345. The method of embodiment 1323, wherein the proliferative disease is malignant fibrous histiocytoma.
1346. The method of embodiment 1323, wherein the proliferative disease is germ cell tumor.
1347. The method of embodiment 1323, wherein the proliferative disease is gallbladder cancer.
1348. The method of embodiment 1323, wherein the proliferative disease is gastric cancer.
1349. The method of embodiment 1323, wherein the proliferative disease is gastrointestinal carcinoid tumor.
1350. The method of embodiment 1323, wherein the proliferative disease is gastrointestinal stromal tumor.
1351. The method of embodiment 1323, wherein the proliferative disease is gestational trophoblastic disease.
1352. The method of embodiment 1323, wherein the proliferative disease is glioma.
1353. The method of embodiment 1323, wherein the proliferative disease is head and neck cancer.
1354. The method of embodiment 1323, wherein the proliferative disease is heart cancer.
1355. The method of embodiment 1323, wherein the proliferative disease is HER2+ cancer.
1356. The method of embodiment 1323, wherein the proliferative disease is hypopharyngeal cancer.
1357. The method of embodiment 1323, wherein the proliferative disease is Kaposi sarcoma.
1358. The method of embodiment 1323, wherein the proliferative disease is kidney cancer.
1359. The method of embodiment 1323, wherein the proliferative disease is Langerhans cell histiocytosis.
1360. The method of embodiment 1323, wherein the proliferative disease is laryngeal cancer.
1361. The method of embodiment 1323, wherein the proliferative disease is lip and oral cavity cancer.
1362. The method of embodiment 1323, wherein the proliferative disease is liver cancer.
1363. The method of embodiment 1323, wherein the proliferative disease is lung cancer.
1364. The method of embodiment 1323, wherein the proliferative disease is mesothelioma.
1365. The method of embodiment 1323, wherein the proliferative disease is metastatic squamous neck cancer with occult primary.
1366. The method of embodiment 1323, wherein the proliferative disease is midline tract carcinoma involving NUT gene.
1367. The method of embodiment 1323, wherein the proliferative disease is mouth cancer.
1368. The method of embodiment 1323, wherein the proliferative disease is nasal cavity cancer.
1369. The method of embodiment 1323, wherein the proliferative disease is nasopharyngeal cancer.
1370. The method of embodiment 1323, wherein the proliferative disease is neuroblastoma.
1371. The method of embodiment 1323, wherein the proliferative disease is oropharyngeal cancer.
1372. The method of embodiment 1323, wherein the proliferative disease is ovarian cancer.
1373. The method of embodiment 1323, wherein the proliferative disease is pancreatic cancer.
1374. The method of embodiment 1323, wherein the proliferative disease is para-nasal sinus cancer.
1375. The method of embodiment 1323, wherein the proliferative disease is paraganglioma.
1376. The method of embodiment 1323, wherein the proliferative disease is parathyroid cancer.
1377. The method of embodiment 1323, wherein the proliferative disease is penile cancer.
1378. The method of embodiment 1323, wherein the proliferative disease is pharyngeal cancer.
1379. The method of embodiment 1323, wherein the proliferative disease is pituitary cancer.
1380. The method of embodiment 1323, wherein the proliferative disease is pleuropulmonary blastoma.
1381. The method of embodiment 1323, wherein the proliferative disease is prostate cancer.
1382. The method of embodiment 1323, wherein the proliferative disease is rectal cancer.
1383. The method of embodiment 1323, wherein the proliferative disease is renal cell cancer.
1384. The method of embodiment 1323, wherein the proliferative disease is renal pelvis and ureter cancer.
1385. The method of embodiment 1323, wherein the proliferative disease is retinoblastoma.
1386. The method of embodiment 1323, wherein the proliferative disease is a rhabdoid tumor.
1387. The method of embodiment 1323, wherein the proliferative disease is salivary gland cancer.
1388. The method of embodiment 1323, wherein the proliferative disease is skin cancer.
1389. The method of embodiment 1323, wherein the proliferative disease is small intestine cancer.
1390. The method of embodiment 1323, wherein the proliferative disease is soft tissue sarcoma.
1391. The method of embodiment 1323, wherein the proliferative disease is spinal cord tumor.
1392. The method of embodiment 1323, wherein the proliferative disease is stomach cancer.
1393. The method of embodiment 1323, wherein the proliferative disease is teratoid tumor.
1394. The method of embodiment 1323, wherein the proliferative disease is testicular cancer.
1395. The method of embodiment 1323, wherein the proliferative disease is throat cancer.
1396. The method of embodiment 1323, wherein the proliferative disease is thymoma.
1397. The method of embodiment 1323, wherein the proliferative disease is thymic carcinoma.
1398. The method of embodiment 1323, wherein the proliferative disease is thyroid cancer.
1399. The method of embodiment 1323, wherein the proliferative disease is urethral cancer.
1400. The method of embodiment 1323, wherein the proliferative disease is uterine cancer.
1401. The method of embodiment 1323, wherein the proliferative disease is vaginal cancer.
1402. The method of embodiment 1323, wherein the proliferative disease is vulvar cancer.
1403. The method of embodiment 1323, wherein the proliferative disease is Wilms tumor.
1404. The method of embodiment 1264, wherein the subject has an autoimmune disorder.
1405. The method of embodiment 1404, wherein the autoimmune disorder is systemic lupus erythematosus (SLE), Sjögren's syndrome, scleroderma, rheumatoid arthritis (RA), juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, pemphigus vulgaris, vitiligo, autoimmune haemolytic anaemia, idiopathic thrombocytopenic purpura, giant cell arteritis, myasthenia gravis, multiple sclerosis (MS) (e.g., relapsing-remitting MS (RRMS)), glomerulonephritis, Goodpasture's syndrome, bullous pemphigoid, colitis ulcerosa, Guillain-Barré syndrome, chronic inflammatory demyelinating polyneuropathy, anti-phospholipid syndrome, narcolepsy, sarcoidosis, or Wegener's granulomatosis.
1406. The method of embodiment 1405, wherein the autoimmune disorder is systemic lupus erythematosus (SLE).
1407. The method of embodiment 1405, wherein the autoimmune disorder is Sjögren's syndrome.
1408. The method of embodiment 1405, wherein the autoimmune disorder is scleroderma.
1409. The method of embodiment 1405, wherein the autoimmune disorder is rheumatoid arthritis (RA).
1410. The method of embodiment 1405, wherein the autoimmune disorder is juvenile idiopathic arthritis.
1411. The method of embodiment 1405, wherein the autoimmune disorder is graft versus host disease.
1412. The method of embodiment 1405, wherein the autoimmune disorder is dermatomyositis.
1413. The method of embodiment 1405, wherein the autoimmune disorder is type I diabetes mellitus.
1414. The method of embodiment 1405, wherein the autoimmune disorder is Hashimoto's thyroiditis.
1415. The method of embodiment 1405, wherein the autoimmune disorder is Graves's disease.
1416. The method of embodiment 1405, wherein the autoimmune disorder is Addison's disease.
1417. The method of embodiment 1405, wherein the autoimmune disorder is celiac disease.
1418. The method of embodiment 1405, wherein the autoimmune disorder is Crohn's Disease.
1419. The method of embodiment 1405, wherein the autoimmune disorder is pernicious anaemia.
1420. The method of embodiment 1405, wherein the autoimmune disorder is pemphigus vulgaris.
1421. The method of embodiment 1405, wherein the autoimmune disorder is vitiligo.
1422. The method of embodiment 1405, wherein the autoimmune disorder is autoimmune haemolytic anaemia.
1423. The method of embodiment 1405, wherein the autoimmune disorder is idiopathic thrombocytopenic purpura.
1424. The method of embodiment 1405, wherein the autoimmune disorder is giant cell arteritis.
1425. The method of embodiment 1405, wherein the autoimmune disorder is myasthenia gravis.
1426. The method of embodiment 1405, wherein the autoimmune disorder is multiple sclerosis (MS).
1427. The method of embodiment 1426, wherein the autoimmune disorder is relapsing-remitting MS (RRMS).
1428. The method of embodiment 1405, wherein the autoimmune disorder is glomerulonephritis.
1429. The method of embodiment 1405, wherein the autoimmune disorder is Goodpasture's syndrome.
1430. The method of embodiment 1405, wherein the autoimmune disorder is bullous pemphigoid.
1431. The method of embodiment 1405, wherein the autoimmune disorder is colitis ulcerosa.
1432. The method of embodiment 1405, wherein the autoimmune disorder is Guillain-Barré syndrome.
1433. The method of embodiment 1405, wherein the autoimmune disorder is chronic inflammatory demyelinating polyneuropathy.
1434. The method of embodiment 1405, wherein the autoimmune disorder is anti-phospholipid syndrome.
1435. The method of embodiment 1405, wherein the autoimmune disorder is narcolepsy.
1436. The method of embodiment 1405, wherein the autoimmune disorder is sarcoidosis.
1437. The method of embodiment 1405, wherein the autoimmune disorder is Wegener's granulomatosis.
1438. The method of any one of embodiments 1264 to 1437, further comprising administering at least one additional agent to the subject.
1439. A nucleic acid or plurality of nucleic acids encoding the CD3 binding molecule of any one of embodiments 1 to 1224.
1440. A cell engineered to express the CD3 binding molecule of any one of embodiments 1 to 1224.
1441. A cell transfected with one or more expression vectors comprising one or more nucleic acid sequences encoding the CD3 binding molecule of any one of embodiments 1 to 1224 under the control of one or more promoters.
1442. A method of producing a CD3 binding molecule, comprising:

(a) culturing the cell of embodiment 1440 or embodiment 1441 in conditions under which the CD3 binding molecule is expressed; and
(b) recovering the CD3 binding molecule from the cell culture.

### 9.2. Numbered Embodiments 1′ to 53′

1′. A multspecific binding molecule (MBM), comprising:
(a) at least one antigen-binding module 1 (ABM1) that binds specifically to human CD3; and
(b) at least one antigen-binding module 2 (ABM2) that binds specifically to specifically to a human tumor-associated antigen (TAA);
wherein each ABM is capable of binding its respective target at the same time as each of the other antigen-binding modules is bound to its respective target.
2′. The MBM of embodiment 1′, wherein ABM1 is:
(a) an immunoglobulin scaffold-based ABM which is optionally an anti-CD3 antibody, an antibody fragment, an scFv, a dsFv, a Fv, a Fab, an scFab, a (Fab′)2, a single domain antibody (SDAB), a VH or VL domain, or a camelid VHH domain; or
(b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.
3′. The MBM of embodiment 2′, wherein ABM1 is a scFv.
4′. The MBM of embodiment 2′, wherein ABM1 is a Fab.
5′. The MBM of embodiment 4′, wherein the Fab is a Fab heterodimer.
6′. The MBM of any one of embodiments 2′ to 5′, wherein ABM1 comprises any of the binding sequences set forth in Table 19.
7′. The MBM of any one of embodiments 1′ to 5′, wherein ABM1 comprises: (i) a heavy chain variable region that comprises (a) a HCDR1 (CDR-Complementarity Determining Region) of SEQ ID NO: 136, (b) a HCDR2 of SEQ ID NO:137, (c) a HCDR3 of SEQ ID NO: 138 and a light chain variable region that comprises: (d) a LCDR1 of SEQ ID NO: 152, (e) a LCDR2 of SEQ ID NO: 153, and (f) a LCDR3 of SEQ ID NO: 154;
(ii) a heavy chain variable region that comprises (a) a HCDR1 of SEQ ID NO: 168, (b) a HCDR2 of SEQ ID NO: 169, (c) a HCDR3 of SEQ ID NO: 170; and a light chain variable region that comprises: (d) a LCDR1 of SEQ ID NO: 184, (e) a LCDR2 of SEQ ID NO: 185, and (f) a LCDR3 of SEQ ID NO: 186;
(iii) a heavy chain variable region that comprises (a) a HCDR1 of SEQ ID NO: 200, (b) a HCDR2 of SEQ ID NO: 201, (c) a HCDR3 of SEQ ID NO: 202; and a light chain variable region that comprises: (d) a LCDR1 of SEQ ID NO: 216, (e) a LCDR2 of SEQ ID NO: 217, and (f) a LCDR3 of SEQ ID NO: 218;
(iv) a heavy chain variable region that comprises: (a) a HCDR1 of SEQ ID NO: 232, (b) a HCDR2 of SEQ ID NO: 233, (c) a HCDR3 of SEQ ID NO: 234; and a light chain variable region that comprises: (d) a LCDR1 of SEQ ID NO: 248, (e) a LCDR2 of SEQ ID NO: 249, and (f) a LCDR3 of SEQ ID NO: 250;
(v) a heavy chain variable region that comprises: (a) a HCDR1 of SEQ ID NO: 264, (b) a HCDR2 of SEQ ID NO: 265, (c) a HCDR3 of SEQ ID NO: 266; and a light chain variable region that comprises: (d) a LCDR1 of SEQ ID NO: 280, (e) a LCDR2 of SEQ ID NO: 281, and (f) a LCDR3 of SEQ ID NO: 282;
(vi) a heavy chain variable region that comprises: (a) a HCDR1 of SEQ ID NO: 296, (b) a HCDR2 of SEQ ID NO: 297, (c) a HCDR3 of SEQ ID NO: 298; and a light chain variable region that comprises: (d) a LCDR1 of SEQ ID NO: 312, (e) a LCDR2 of SEQ ID NO: 313, and (f) a LCDR3 of SEQ ID NO: 314;
(vii) a heavy chain variable region that comprises: (a) a HCDR1 of SEQ ID NO: 328, (b) a HCDR2 of SEQ ID NO: 329, (c) a HCDR3 of SEQ ID NO: 330; and a light chain variable region that comprises: (d) a LCDR1 of SEQ ID NO: 344, (e) a LCDR2 of SEQ ID NO: 345, and (f) a LCDR3 of SEQ ID NO: 346;
(viii) a heavy chain variable region that comprises: (a) a HCDR1 of SEQ ID NO: 360, (b) a HCDR2 of SEQ ID NO: 361, (c) a HCDR3 of SEQ ID NO: 362; and a light chain variable region that comprises: (d) a LCDR1 of SEQ ID NO: 376, (e) a LCDR2 of SEQ ID NO: 377, and (f) a LCDR3 of SEQ ID NO: 378;
(ix) a heavy chain variable region that comprises: (a) a HCDR1 of SEQ ID NO: 392, (b) a HCDR2 of SEQ ID NO: 393, (c) a HCDR3 of SEQ ID NO: 394; and a light chain variable region that comprises: (d) a LCDR1 of SEQ ID NO: 408, (e) a LCDR2 of SEQ ID NO:409, and (f) a LCDR3 of SEQ ID NO:410; or
(x) a heavy chain variable region that comprises: (a) a HCDR1 of SEQ ID NO: 424, (b) a HCDR2 of SEQ ID NO: 425, (c) a HCDR3 of SEQ ID NO: 426; and a light chain variable region that comprises: (d) a LCDR1 of SEQ ID NO: 440, (e) a LCDR2 of SEQ ID NO: 441, and (f) a LCDR3 of SEQ ID NO:442.
8′. The MBM of any one of embodiments 1′ to 5′, wherein ABM1 comprises:
(i) a heavy chain variable region (vH) that comprises SEQ ID NO: 145, and a light chain variable region (vL) that comprises SEQ ID NO:161;
(ii) a heavy chain variable region (vH) that comprises SEQ ID NO: 177, and a light chain variable region (vL) that comprises SEQ ID NO: 193;
(iii) a heavy chain variable region (vH) that comprises SEQ ID NO: 209, and a light chain variable region (vL) that comprises SEQ ID NO: 225;
(iv) a heavy chain variable region (vH) that comprises SEQ ID NO: 241, and a light chain variable region (vL) that comprises SEQ ID NO: 257;
(v) a heavy chain variable region (vH) that comprises SEQ ID NO: 273, and a light chain variable region (vL) that comprises SEQ ID NO: 289;
(vi) a heavy chain variable region (vH) that comprises SEQ ID NO: 305, and a light chain variable region (vL) that comprises SEQ ID NO: 321;
(vii) a heavy chain variable region (vH) that comprises SEQ ID NO: 337, and a light chain variable region (vL) that comprises SEQ ID NO: 353;
(viii) a heavy chain variable region (vH) that comprises SEQ ID NO: 369, and a light chain variable region (vL) that comprises SEQ ID NO: 385;
(ix) a heavy chain variable region (vH) that comprises SEQ ID NO: 401, and a light chain variable region (vL) that comprises SEQ ID NO: 417; or
(x) a heavy chain variable region (vH) that comprises SEQ ID NO: 433, and a light chain variable region (vL) that comprises SEQ ID NO: 449.
9′. The MBM of embodiment 8′, that retains at least 90, 91, 92, 93, 94, 95, 96, 97, 98 or 99% identity over either the variable light or variable heavy region.
10′. The MBM of embodiment 1′, wherein ABM2 is:
(a) an immunoglobulin scaffold-based ABM which is an anti-TAA antibody, an antibody fragment, an scFv, a dsFv, a Fv, a Fab, an scFab, a (Fab′)2, a single domain antibody (SDAB), a VH or VL domain, or a camelid VHH domain; or
(b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.
11′. The MBM of embodiment 10, wherein the TAA is TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, EGFRvIII, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, FLT3, TAAG72, CD22, CD33, GD2, BCMA, gp100Tn, FAP, tyrosinase, EPCAM, CEA, Igf-I receptor, Cadherin17, CD32b, GPNMB, GPR64, HER3, LRP6, LYPD8, NKG2D, SLC34A2, SLC39A6, SLITRK6, TACSTD2, or EphB2.
12′. The MBM of embodiment 10′, wherein ABM2 comprises the CDRs or variable region sequences of the antibodies set forth in Table 15A.
13′. The MBM of embodiments 10′ or 11′, wherein the TAA is BCMA.
14′. The MBM of embodiments 10′ or 11′, wherein the TAA is CD19.
15′. The MBM of embodiment 14′, wherein ABM2 comprises any of the binding sequences set forth Table 17.
16′. The MBM of any one of embodiments 10′ to 15′, wherein wherein ABM2 is an scFv.
17′. The MBM of any one of embodiments 10′ to 15′, wherein ABM2 is a Fab.
18′. The MBM of embodiment 17′, wherein the Fab is a Fab heterodimer.
19′. The MBM of any one of embodiments 1′ to 18′, which comprises an Fc domain.
20′. The MBM of embodiment 19′, wherein the Fc domain is an Fc heterodimer.
21′. The MBM of embodiment 20′, wherein the Fc heterodimer comprises any of the Fc modifications set forth in Table 4.
22′. The MBM of any one of embodiments 19′ to 21′, wherein the Fc domain has altered effector function.
23′. The MBM of embodiment 22′, wherein the Fc domain has altered binding to one or more Fc receptors.
24′. The MBM of embodiment 23′, wherein the one or more Fc receptors comprise FcRN.
25′. The MBM of embodiment 23′, wherein the one or more Fc receptors comprise leukocyte receptors.
26′. The MBM of any one of embodiments 19′ to 25′, wherein the Fc has modified disulfide bond architecture.
27′. The MBM of any one of embodiments 19′ to 26′, wherein the Fc has altered glycosylation patterns.
28′. The MBM of any one of embodiments 19′ to 27′, wherein the Fc comprises a hinge region.
29′. The MBM of embodiment 28′ wherein the hinge region is set forth in Table 7.
30′. The MBM of any one of embodiments 1′ to 29′, which comprises at least one scFv domain.
31′. The MBM of embodiment 30′, wherein at least one scFv comprises a linker connecting the VH and VL domains.
32′. The MBM of embodiment 31′, wherein the linker is 5 to 25 amino acids in length.
33′. The MBM of embodiment 32′, wherein the linker is 12 to 20 amino acids in length.
34′. The MBM of any one of embodiments 31′ to 33′, wherein the linker is a charged linker and/or a flexible linker.
35′. The MBM of any one of embodiments 31′ to 34′, wherein the linker is selected from any one of linkers L1 through L54 (SEQ ID NO:25-78)
36′. The MBM of any one of embodiments 1′ to 29′, which comprises at least one Fab domain.
37′. The MBM of embodiment 36′, wherein at least one Fab domain comprises any of the Fab heterodimerization modifications set forth in Table 2.
38′. A multspecific binding molecule (MBM), comprising:
(a) at least one antigen-binding module 1 (ABM1) that binds specifically to human CD3;
(b) at least one antigen-binding module 2 (ABM2) that binds specifically to a human tumor-associated antigen (TAA); and
(c) at least one antigen binding module 3 (ABM3) that bind specifically to an immunoglobulin scaffold-based ABM which is an anti-CD2 antibody, an antibody fragment, an scFv, a dsFv, a Fv, a Fab, an scFab, a (Fab′)2, a single domain antibody (SDAB), a VH or VL domain, or a camelid VHH domain or a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.
39′. The MBM of embodiment 38′, wherein ABM3 comprises any of the binding sequences set forth in Table 13 or Table 14.
40′. The MBM of embodiment 39′, wherein ABM3 is a scFv.
41′. The MBM of embodiment 39′, wherein ABM3 is a Fab.
42′. A conjugate comprising the MBM of any one of embodiments 1′ to 41′ and a cytotoxic or cytostatic agent.
43′. The conjugate of embodiment 42′, wherein the cytotoxic or cytostatic agent is conjugated to the MBM via a linker.
44′. A pharmaceutical composition comprising the MBM of any one of embodiments 1′ to 41′ or the conjugate of embodiment 42′ or embodiment 43′ and an excipient.
45′. A method of treating a subject with cancer, comprising administering to a subject suffering from cancer an effective amount of the MBM of any one of embodiments 1′ to 41′, the conjugate of embodiment 42′ or embodiment 43′, or the pharmaceutical composition of embodiment 44′.
46′. The method of embodiment 45′, wherein the cancer is selected from HER2+ cancer, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.
47′. The method of 46′, further comprising administering at least one additional agent to the subject.
48′. A nucleic acid or plurality of nucleic acids encoding the MBM of any one of embodiments 1′ to 41′.
49′. A cell engineered to comprise a nucleic acid or plurality of nucleic acids encoding the MBM of any one of embodiments 1′ to 41′.
50′. The cell of embodiment 49′ transfected with one or more expression vectors comprising one or more nucleic acid sequences encoding the MBM under the control of one or more promoters.
51′. The cell of embodiment 50′, wherein the one or more promoters comprises an inducible promoter.
52′. The cell of any one of embodiments 49′ to 51′, wherein the MBM is produced in secretable form.
53′. A method of producing a MBM, comprising:
(a) culturing the cell of any one of embodiments 49′ to 51′ in conditions under which the MBM is expressed; and
(b) recovering the MBM from the cell culture.

1. A CD3 binding molecule that specifically binds to human CD3 and comprises a CDR-H1 sequence, a CDR-H2 sequence a CDR-H3 sequence, a CDR-L1 sequence, a CDR-L2 sequence, and a CDR-L3 sequence set forth in Table 1A, Table 1B, or Table 1C.

2-4. (canceled)

5. The CD3 binding molecule of claim 1, comprises CDR-H1 CDR-H2, and CDR-H3 sequences set forth in Table 1D-1, Table 1E-1, Table 1F-1, Table 1G-1, Table 1H-1, or Table 1I-1, and the corresponding CDR-L1, CDR-L2, and CDR-L3 sequences set forth in Table 1D-2, Table 1E-2, Table 1F-2, Table 1G-2, Table 1H-2, or Table 1I-2, respectfully.

6-21. (canceled)

22. The CD3 binding molecule of claim 5, which comprises a heavy chain variable sequence set forth in Table 1J-1 and the corresponding light chain variable sequence set forth in Table 1J-2.

23. The CD3 binding molecule of claim 1, which comprises an antibody, an antibody fragment, an scFv, a dsFv, a Fv, a Fab, an scFab, a (Fab′)2, or a single domain antibody (SDAB).

24-25. (canceled)

26. The CD3 binding molecule of claim 1, which is a multispecific binding molecule.

27. The CD3 binding molecule of claim 26, which is a bispecific binding molecule (BBM).

28. The CD3 binding molecule of claim 27, wherein the BBM comprises:
(a) an antigen binding module 1 (ABM1) that binds specifically to CD3; and comprises heavy and light chain variable regions of the CD3 binding molecule of claim 1; and
(b) an antigen binding module 2 (ABM2) that binds specifically to a tumor-associated antigen (“TAA”).

29-32. (canceled)

33. The CD3 binding molecule of claim 26, which is a trispecific binding molecule (TBM).

34. The CD3 binding molecule of claim 29-32, wherein the TBM comprises:
(a) an antigen binding module 1 (ABM1) that binds specifically to CD3 and comprises heavy and light chain variable regions of the CD3 binding molecule of claim 1; and
(b) an antigen binding module 2 (ABM2) that binds specifically to a tumor-associated antigen; and
(c) an antigen binding module 3 (ABM3) that binds specifically to:
(i) a tumor-associated antigen other than the tumor-associated antigen bound by ABM2; or
(ii) CD2.

35. The CD3 binding molecule of claim 34, in which ABM1 is capable of binding CD3 at the same time ABM2 and ABM3 are bound to their target molecules.

36-39. (canceled)

40. The CD3 binding molecule of claim 34, wherein ABM2 specifically binds a TAA which is CD19, BCMA, TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD20, CD30, ERBB2, ROR1, FLT3, TAAG72, CD22, CD33, GD2, gp100Tn, FAP, tyrosinase, EPCAM, CEA, Igf-I receptor, EphB2, mesothelin, Cadherin17, CD32b, EGFRvIII, GPNMB, GPR64, HER3, LRP6, LYPD8, NKG2D, SLC34A2, SLC39A6, SLITRK6, or TACSTD2.

41. The CD3 binding molecule of claim 40, wherein ABM3 binds CD2.

42-43. (canceled)

44. A conjugate comprising the CD3 binding molecule of claim 1 and an agent.

45. A pharmaceutical composition comprising the CD3 binding molecule of claim 1 and a pharmaceutically acceptable excipient.

46. A method of treating a subject having a proliferative disease or an autoimmune disorder comprising administering to the subject the CD3 binding molecule of claim 1.

47. A nucleic acid or plurality of nucleic acids encoding the CD3 binding molecule of claim 1.

48. A cell engineered to express the CD3 binding molecule of claim 1.

49. A method of producing a CD3 binding molecule, comprising:
(a) culturing the cell of claim 48 in conditions under which the CD3 binding molecule is expressed; and
(b) recovering the CD3 binding molecule from the cell culture.
